(19) United States
(12) Patent Application Publication
(10) Pub. No.: US 2025/0161459 A1
(43) Pub. Date: May 22, 2025

(54) TRKB LIGAND CONJUGATED COMPOUNDS AND USES THEREOF

(71) Applicant: ADARx Pharmaceuticals, Inc., San Diego, CA (US)

(72) Inventors: Chandramouli Chiruta, San Diego, CA (US); Huey-jing Huang, San Diego, CA (US); Zhen Li, San Diego, CA (US); Rui Zhu, San Diego, CA (US); Mihai Azimioara, San Diego, CA (US); Bo Cheng, San Diego, CA (US)

(73) Assignee: ADARx Pharmaceuticals, Inc., San Diego, CA (US)

(21) Appl. No.: 18/837,377

(22) PCT Filed: Feb. 10, 2023
(86) PCT No.: PCT/US2023/062436

§ 371 Date: Aug. 9, 2024

Related U.S. Application Data
(60) Provisional application No. 63/352,809, filed on Jun. 16, 2022.
(60) Provisional application No. 63/335,150, filed on Apr. 26, 2022.
(60) Provisional application No. 63/327,342, filed on Apr. 4, 2022.
(60) Provisional application No. 63/309,337, filed on Feb. 11, 2022.

(51) Int. Cl. A61K 47/54; A61K 47/60; A61K 47/64; A61K 47/68; A61P 25/28

(52) U.S. Cl. CPC A61K 47/548; A61K 47/545; A61K 47/549; A61K 47/60; A61K 47/64; A61K 47/6889; A61P 25/28

(57) ABSTRACT

Provided herein are TrkB ligand-containing compounds, methods of delivering said compounds, and methods of treating diseases, disorders, and symptoms (e.g., central nervous system diseases, disorders, and symptoms) in a subject using said compounds.

### CROSS-REFERENCE TO RELATED APPLICATIONS

This application claims the benefit of priority under 35 U.S.C. § 119 (e) of U.S. Provisional Application No. 63/309,337, filed Feb. 11, 2022; U.S. Provisional Application No. 63/327,342, filed Apr. 4, 2022; U.S. Provisional Application No. 63/335,150, filed Apr. 26, 2022; and U.S. Provisional Application No. 63/352,809, filed Jun. 16, 2022. The disclosure of each of the prior applications is considered part of and is incorporated by reference in its entirety in the disclosure of this application.

### BACKGROUND

In the use of compounds in therapeutic, prophylactic, or diagnostic applications, it is often desirable that the compounds be delivered to a specific location (for example, to desired cell(s)) to enhance the therapeutic or prophylactic effect or to be advantageous for diagnostic purposes. This is frequently the case when attempting to deliver a therapeutic compound in vivo. Further, being able to efficiently deliver a compound to a specific location can limit or potentially eliminate unintended consequences (such as off-target effects) that may be caused by administration of the compound. One strategy to facilitate delivery of a compound, such as a therapeutic, prophylactic, or diagnostic compound, to a desired location in vivo, is by linking or attaching the compound to a targeting ligand.

One class of compounds that can be targeted using targeting ligands are oligomeric compounds such as, for example, proteins, peptides, antibodies, and oligonucleotides. Oligomeric compounds that include nucleotide sequences (e.g., oligonucleotides) at least partially complementary to a target nucleic acid have been shown to alter the function and activity of the target both in vitro and in vivo. When delivered to a cell containing a target nucleic acid (such as mRNA or pre-mRNA), oligonucleotides have been shown to modulate the expression or activity of the target nucleic acid. In certain instances, the oligonucleotide can reduce the expression of the gene by inhibiting translation of the nucleic acid target and/or triggering the degradation of the target nucleic acid.

If the target nucleic acid is mRNA, one mechanism by which an oligonucleotide can modulate the expression of the mRNA target is through RNA interference. RNA interference is a biological process by which RNA or RNA-like molecules (such as chemically modified RNA molecules) are able to silence gene expression, at least in part, through the RNA-induced silencing Complex (RISC) pathway. Additionally, oligonucleotides can modulate the expression of a target nucleic acid, such as a target mRNA, through an RNase recruitment mechanism, microRNA mechanisms, occupancy-based mechanisms, and editing mechanisms. Oligonucleotides may be single-stranded or double-stranded. Oligonucleotides may comprise DNA, RNA, and RNA-like molecules, which can also include modified nucleosides including one or more non-phosphodiester linkages.

Another class of compounds that can be targeted using targeting ligands are small molecule compounds. The small molecule compounds (e.g., an organic compound having a molecular weight of ca. 1000 daltons or less) are typically shown to alter the function and/or activity of the target such that disease and/or disease symptoms are modulated or ameliorated or are typically useful as a diagnostic marker when localized to the target. More efficient delivery of a compound to a specific location can limit or potentially eliminate unintended consequences (such as off-target effects) that may be caused by administration of the compound and provide improved localization of a diagnostic compound.

### SUMMARY

Embodiments provided herein are directed to compounds (e.g., any of those delineated herein) and methods for targeting cells expressing Tropomyosin Receptor Kinase B (TrkB). Certain embodiments provided herein are directed to compounds and methods for delivering an agent to cells expressing TrkB. In certain embodiments, the cell is in the brain. In certain embodiments, the cell is in the frontal cortex. In certain embodiments, the cell is in the striatum. In certain embodiments, the cell is in the cerebellum. In certain embodiments, the cell is in the brain stem. In certain embodiments, the cell is in the hippocampus. In certain embodiments, the cell is in the spinal cord. In certain embodiments, the agent is a therapeutic compound. In certain embodiments, delivery of the agent is for the treatment of diseases, disorders, and symptoms in a subject. In certain embodiments, the agent is a diagnostic compound. In certain embodiments, a compound comprises a TrkB ligand and one or more linker moieties for attachment to a therapeutic, prophylactic, or diagnostic agent. In certain embodiments, a compound comprises a TrkB ligand, one or more linker moieties, and a therapeutic agent. In certain embodiments, the therapeutic agent is selected from a small molecule or oligomeric compound. In certain embodiments, the oligomeric compound is a protein, peptide, antibody, oligonucleotide, or combination thereof. In certain embodiments, the TrkB ligand is a TrkB agonist. In certain embodiments, the TrkB ligand is a small molecule, aptamer, peptide, or antibody. In certain embodiments, the TrkB ligand is a flavone, tropoflavin, or derivative thereof. In certain embodiments, the TrkB ligand is ANA-12. In certain embodiments, the TrkB ligand is any of those delineated herein or a prodrug thereof.

In certain embodiments, contacting a cell expressing TrkB, such as a brain cell, with a compound provided herein, delivers the agent to the cell. In certain embodiments, contacting a cell expressing TrkB, such as a brain cell, with a compound provided herein, treats a disease, disorder, or symptom in a subject. In certain embodiments, a compound comprising a TrkB ligand selectively or preferentially targets a cell expressing TrkB compared to a cell not expressing TrkB. In certain embodiments, a compound comprising a TrkB ligand selectively or preferentially targets a cell expressing TrkB compared to a compound not comprising a TrkB ligand.

Certain embodiments provided herein are directed to compounds and methods for modulating expression of a nucleic acid target in cells expressing TrkB. In certain embodiments, the cell is in the brain. In certain embodiments, the cell is in the frontal cortex. In certain embodiments, the cell is in the striatum. In certain embodiments, the cell is in the cerebellum. In certain embodiments, the cell is in the brain stem. In certain embodiments, the cell is in the hippocampus. In certain embodiments, the cell is in the spinal cord. In certain embodiments, contacting a cell expressing TrkB, such as a brain cell, with a compound provided herein, modulates the expression or activity of a nucleic acid target in the cell. In certain embodiments, a compound comprises a TrkB ligand, one or more linker moieties, and an oligonucleotide.

It is understood that the embodiments provided herein with respect to preferred variable selections can be taken alone or in combination with one or more embodiments, or other preferred variable selections provided herein, as if each combination were explicitly listed herein.

In one aspect, the present disclosure provides compounds, or a stereoisomer, tautomer, prodrug, or salt thereof, of Formula (V):

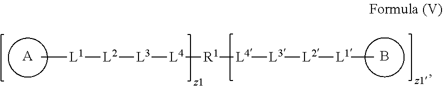

wherein:
each

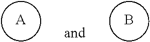

are independently a Tropomyosin receptor B (TrkB) ligand;
each of L¹, L², L³, L⁴, L^{1′}, L^{2′}, L^{3′}, and L^{4′} is independently a linker, bond, or absent;
R¹ is an oligonucleotide, a protecting group, a small molecule, a protein, an antibody, or a peptide;
z1 is 1, 2, or 3; and
z1′ is 0, 1, 2, or 3.

In one aspect, the present disclosure provides compounds, or a stereoisomer, tautomer, prodrug, or salt thereof, of Formula (I):

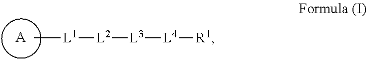

wherein

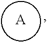

L¹, L², L³, L⁴, and R¹ are as described herein.

In one aspect, the present disclosure provides compounds, or a stereoisomer, tautomer, prodrug, or salt thereof, of Formula (I′):

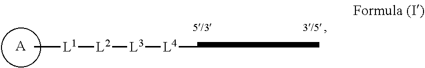

wherein:
[symbol] is an oligonucleotide and

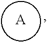

L¹, L², L³, and L⁴ are as described herein.

In one aspect, the present disclosure provides compounds, or a stereoisomer, tautomer, prodrug, or salt thereof, of Formula (VI):

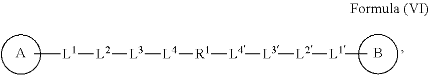

wherein:

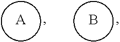

L¹, L², L³, L⁴, L^{1′}, L^{2′}, L^{3′}, L^{4′} and R¹ are as described herein.

In one aspect, the present disclosure provides compounds, or a stereoisomer, tautomer, prodrug, or salt thereof, of Formula (VI′):

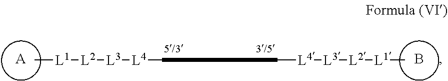

wherein:
[symbol] ,

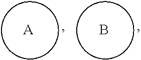

L¹, L², L³, L⁴, L^{1′}, L^{2′}, L^{3′}, and L^{4′} are as described herein.

In one aspect, the present disclosure provides compounds, or a stereoisomer, tautomer, prodrug, or salt thereof, of Formula (II):

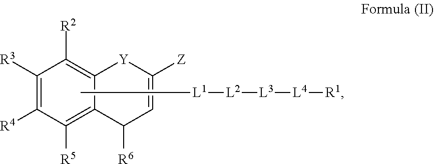

wherein:
R¹ is an oligonucleotide, a protecting group, a small molecule, a protein, an antibody, or a peptide;
R² is hydrogen, —OR⁷, —SR⁸, or —NR⁹R¹⁰;
R³ is hydrogen, —OR³¹, —SR³², or —NR³³R³⁴;
R⁴ is hydrogen, —OR³⁵, —SR³⁶, or —NR³⁷R³⁸;
R⁵ is hydrogen, —OR³⁹, —SR⁴⁰, or —NR⁴¹R⁴²;
R⁶ is hydrogen, —OH, optionally substituted —O-alkyl, optionally substituted —OAc, —NH₂, optionally substituted —NHAc, —SH, or ═O;
R⁷, R⁸, R⁹, R¹⁰, R³¹, R³², R³³, R³⁴, R³⁵, R³⁶, R³⁷, R³⁸, R³⁹, R⁴⁰, R⁴¹, and R⁴² are each independently hydrogen, optionally substituted alkyl, optionally substituted heteroalkyl, optionally substituted cycloalkyl, optionally substituted heterocycloalkyl, optionally substituted aryl, optionally substituted heteroaryl;
Y is CH₂, NH, S, or O; and
Z is optionally substituted aryl or optionally substituted heteroaryl.

In certain embodiments, R⁷, R⁸, R⁹, R¹⁰, R³¹, R³², R³³, R³⁴, R³⁵, R³⁶, R³⁷, R³⁸, R³⁹, R⁴⁰, R⁴¹ and R⁴² are each independently optionally substituted unsaturated or partially unsaturated alkyl. In certain embodiments, R⁷, R⁸, R⁹, and R¹⁰ are each independently alkenyl. In certain embodiments, R⁷, R⁸, R⁹, and R¹⁰ are each independently alkynyl.

In certain embodiments, R² is OR⁷. In certain embodiments, R³ is OR³¹. In certain embodiments, R⁷ and R³¹ are each independently hydrogen, optionally substituted alkyl, or optionally substituted alkenyl. In certain embodiments, one or both of R⁷ and R³¹ are independently hydrogen. In certain embodiments, one or both of R⁷ and R³¹ are independently optionally substituted alkyl. In certain embodiments, one or both of R⁷ and R³¹ are independently optionally substituted unsaturated or partially unsaturated alkyl. In certain embodiments, one or both of R⁷ and R³¹ are independently alkenyl. In certain embodiments, R⁷ is optionally substituted alkyl and R³¹ is hydrogen. In certain embodiments, R⁷ is hydrogen and R³¹ is optionally substituted alkyl. In certain embodiments, R⁷ is alkenyl and R³¹ is hydrogen. In certain embodiments, R⁷ is hydrogen and R³¹ is optionally substituted alkenyl.

In one aspect, the present disclosure provides compounds, or a stereoisomer, tautomer, prodrug, or salt thereof, of Formula (II-a):

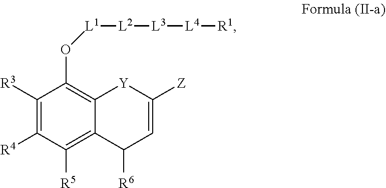

wherein L¹, L², L³, L⁴, R¹, R³, R⁴, R⁵, R⁶, Y, and Z are as described herein.

In one aspect, the present disclosure provides compounds, or a stereoisomer, tautomer, prodrug, or salt thereof, of Formula (II-b):

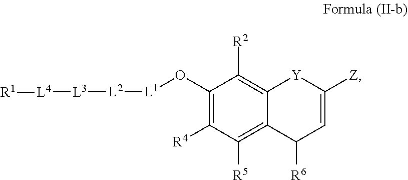

wherein L¹, L², L³, L⁴, R¹, R², R⁴, R⁵, R⁶, Y, and Z are as described herein.

In one aspect, the present disclosure provides compounds, or a stereoisomer, tautomer, prodrug, or salt thereof, of Formula (II-c):

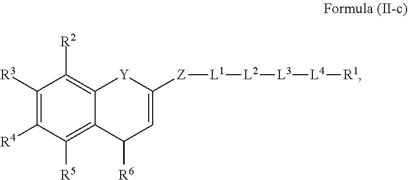

wherein L¹, L², L³, L⁴, R¹, R², R³, R⁴, R⁵, R⁶, Y, and Z are as described herein.

In one aspect, the present disclosure provides compounds, or a stereoisomer, tautomer, prodrug, or salt thereof, of Formula (III):

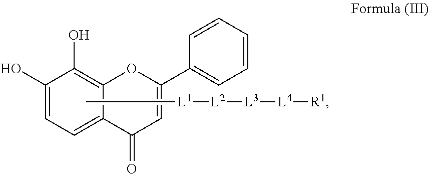

wherein L¹, L², L³, L⁴, and R¹ are as described herein.

In one aspect, the present disclosure provides compounds, or a stereoisomer, tautomer, prodrug, or salt thereof, of Formula (III-a):

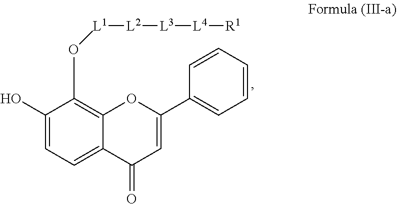

wherein L¹, L², L³, L⁴, and R¹ are as described herein.

In one aspect, the present disclosure provides compounds, or a stereoisomer, tautomer, prodrug, or salt thereof, of Formula (III-b):

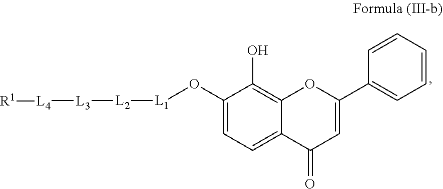

wherein L¹, L², L³, L⁴, and R¹ are as described herein.

In one aspect, the present disclosure provides compounds, or a stereoisomer, tautomer, prodrug, or salt thereof, of Formula (III-c):

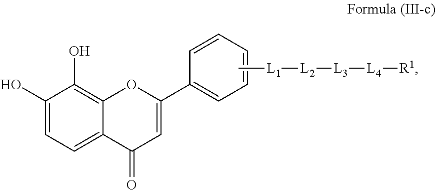

wherein L¹, L², L³, L⁴, and R¹ are as described herein.

In one aspect, the present disclosure provides compounds, or a stereoisomer, tautomer, prodrug, or salt thereof, of Formula (III-c-1):

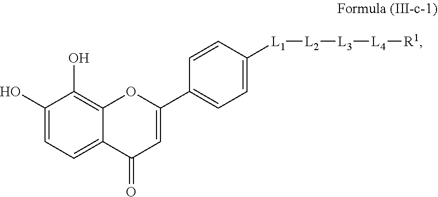

wherein L¹, L², L³, L⁴, and R¹ are as described herein.

In one aspect, the present disclosure provides compounds, or a stereoisomer, tautomer, prodrug, or salt thereof, of Formula (II′):

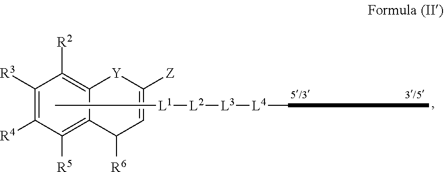

wherein L¹, L², L³, L⁴, R¹, R², R³, R⁴, R⁵, R⁶, Y, and Z are as described herein.

In one aspect, the present disclosure provides compounds, or a stereoisomer, tautomer, prodrug, or salt thereof, of Formula (II″):

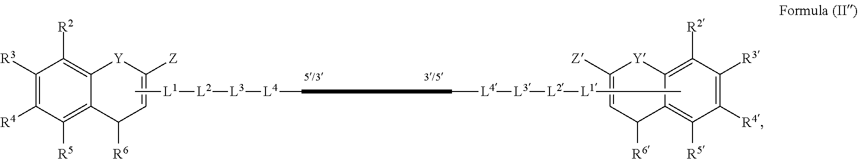

wherein L¹, L², L³, L⁴, L^{1′}, L^{2′}, L^{3′}, L^{4′}, R¹, R², R³, R⁴, R⁵, R⁶, Y, and Z are as described herein;
R^{2′} is hydrogen, —OR^{7′}, —SR^{8′}, or —NR^{9′}R^{10′};
R^{3′} is hydrogen, —OR^{31′}, —SR^{32′}, or —NR^{33′}R^{34′};
R^{4′} is hydrogen, —OR^{35′}, —SR^{36′}, or —NR^{37′}R^{38′};
R^{5′} is hydrogen, —OR^{39′}, —SR^{40′}, or —NR^{41′}R^{42′};
R^{6′} is hydrogen, —OH, optionally substituted —O-alkyl, optionally substituted —OAc, —NH₂, optionally substituted —NHAc, —SH, or ═O;
R^{7′}, R^{8′}, R^{9′}, R^{10′}, R^{31′}, R^{32′}, R^{33′}, R^{34′}, R^{35′}, R^{36′}, R^{37′}, R^{38′}, R^{39′}, R^{40′}, R^{41′}, and R^{42′} are each independently hydrogen, optionally substituted alkyl, optionally substituted heteroalkyl, optionally substituted cycloalkyl, optionally substituted heterocycloalkyl, optionally substituted aryl, optionally substituted heteroaryl;
Y′ is CH₂, NH, S, or O; and
Z′ is optionally substituted aryl or optionally substituted heteroaryl.

In one aspect, the present disclosure provides compounds, or a stereoisomer, tautomer, prodrug, or salt thereof, of Formula (IV):

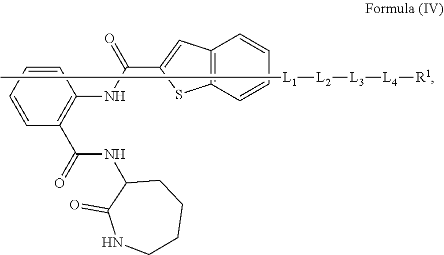

wherein L¹, L², L³, L⁴, and R¹ are as described herein.

In one aspect, the present disclosure provides compounds, or a stereoisomer, tautomer, prodrug, or salt thereof, of Formula (IV-a):

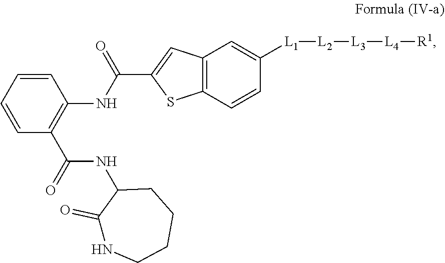

In one aspect, the present disclosure provides compounds, or a stereoisomer, tautomer, prodrug, or salt thereof, of Formula (IV-b):

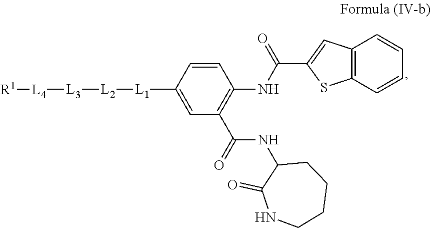

wherein L¹, L², L³, L⁴, and R¹ are as described herein.

In one aspect, the present disclosure provides compounds, or a stereoisomer, tautomer, prodrug, or salt thereof, of Formula (VII):

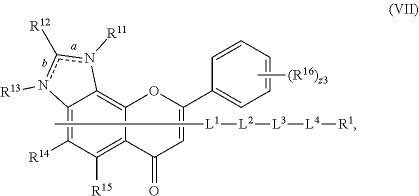

wherein:
L¹, L², L³, L⁴, and R¹ are as described herein;
R¹¹ and R¹³ are each independently absent, hydrogen, or optionally substituted alkyl;
R¹², R¹⁴, and R¹⁵ are each independently hydrogen, optionally substituted alkyl, optionally substituted heteroalkyl, optionally substituted cycloalkyl, optionally substituted heterocycloalkyl, optionally substituted aryl, or optionally substituted heteroaryl;
R¹⁶ is hydrogen, halogen, —CN, —N₃, —SOₙ₁₆R^{1A}, —SOᵥ₁₆NR^{16B}R^{16C}, —NHNR^{16B}R^{16C}, —ONR^{16B}R^{16C}, —NHC(O)NHNR^{16B}R^{16C}, —NHC(O)NR^{16B}R^{16C}, —N(O)ₘ₁₆, —NR^{16B}R^{16C}, —C(O)R^{16D}, —C(O)OR^{16D}, —C(O)NR^{16B}R^{16C}, —OR^{16A}, —NR^{16B}SO₂R^{16A}, —NR^{16B}C(O)R^{16D}, —NR^{16B}C(O)OR^{16D}, —NR^{16B}OR^{16D}, optionally substituted alkyl, optionally substituted heteroalkyl, optionally substituted cycloalkyl, optionally substituted heterocycloalkyl, optionally substituted aryl, or optionally substituted heteroaryl;
[symbol] and [symbol] are each independently a single bond or a double bond, wherein if [symbol] is a single bond, then [symbol] is a double bond and R¹³ is absent; and further wherein if [symbol] is a single bond, then [symbol] is a double bond and R¹¹ is absent;
R^{16A}, R^{16B}, R^{16C}, R^{16D} are each independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{16B} and R^{16C} substituents bonded to the same nitrogen atom may optionally be joined to form a substituted or unsubstituted heterocycloalkyl or substituted or unsubstituted heteroaryl;
z3 is 0, 1, 2, 3, 4, or 5;
n16 is 0, 1, 2, 3, or 4; and
v16 and m16 are each independently 1 or 2.

In one aspect, the present disclosure provides compounds, or a stereoisomer, tautomer, prodrug, or salt thereof, of Formula (VII-a), Formula (VII-b), or a mixture thereof:

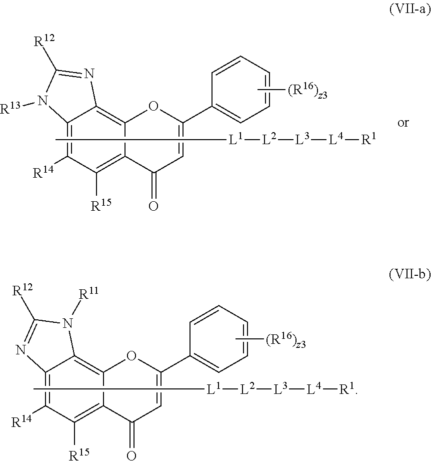

wherein L¹, L², L³, L⁴, R¹, R¹¹, R¹², R¹³, R¹⁴, R¹⁵, R¹⁶, and z3 are as described herein.

In one aspect, the present disclosure provides compounds, or a stereoisomer, tautomer, prodrug, or salt thereof, of Formula (VII-c):

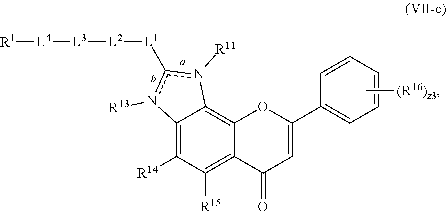

wherein L¹, L², L³, L⁴, R¹, R¹¹, R¹³, R¹⁴, R¹⁵, R¹⁶, z3, [symbol] and [symbol] are as described herein.

In one aspect, the present disclosure provides compounds, or a stereoisomer, tautomer, prodrug, or salt thereof, of Formula (VII-c-1), Formula (VII-c-2), or a mixture thereof:

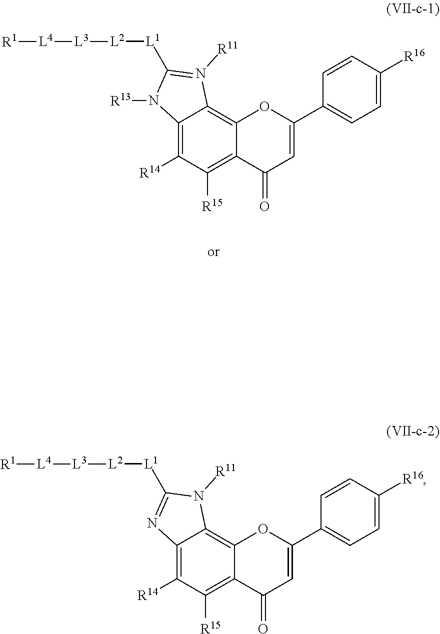

wherein L¹, L², L³, L⁴, R¹, R¹¹, R¹², R¹³, R¹⁴, R¹⁵, and R¹⁶ are as described herein.

In one aspect, the present disclosure provides compounds, or a stereoisomer, tautomer, prodrug, or salt thereof, of Formula (VII-d):

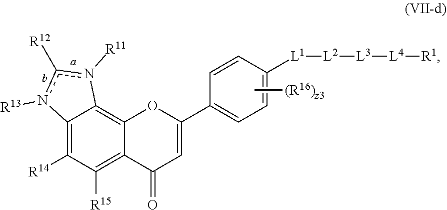

wherein L¹, L², L³, L⁴, R¹, R¹¹, R¹², R¹³, R¹⁴, R¹⁵, R¹⁶, z3, [symbol] , and [symbol] are as described herein.

In one aspect, the present disclosure provides compounds, or a stereoisomer, tautomer, prodrug, or salt thereof, of Formula (VII-d-1), Formula (VII-d-2), or a mixture thereof:

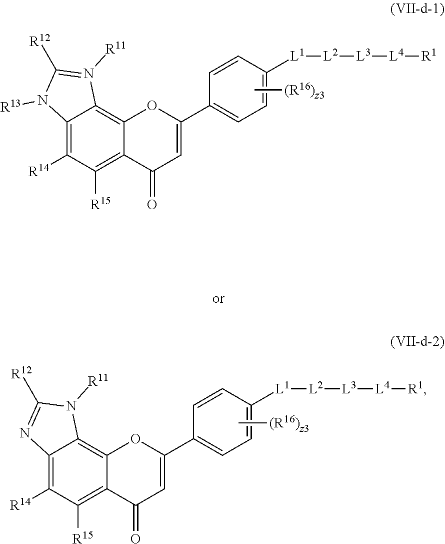

wherein L¹, L², L³, L⁴, R¹, R¹¹, R¹², R¹³, R¹⁴, R¹⁵, R¹⁶, and z3 are as described herein.

In one aspect, the present disclosure provides compounds, or a stereoisomer, tautomer, prodrug, or salt thereof, of Formula (II″):

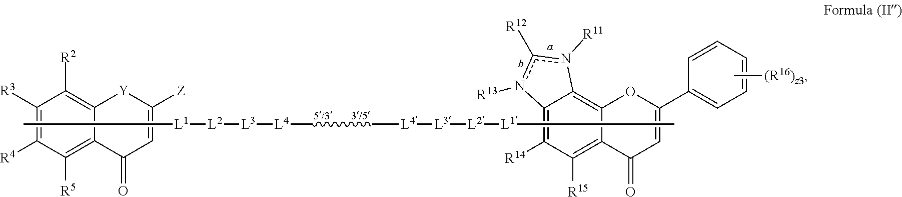

wherein L¹, L², L³, L⁴, L^{1′}, L^{2′}, L^{3′}, L^{4′}, R¹, R², R³, R⁴, R⁵, R⁶, R¹¹, R¹², R¹³, R¹⁴, R¹⁵, R¹⁶, Y, Z, z3, [symbol] , and [symbol] are as described herein.

In one aspect, the present disclosure provides compounds, or a stereoisomer, tautomer, prodrug, or salt thereof, of Formula (II″):

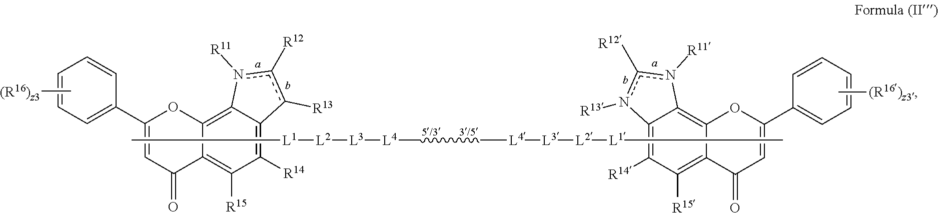

wherein L¹, L², L³, L⁴, L^{1′}, L^{2′}, L^{3′}, L^{4′}, R¹¹, R¹², R¹³, R¹⁴, R¹⁵, R¹⁶, R^{11′}, R^{12′}, R^{13′}, R^{14′}, R^{15′}, R^{16′}, z3, z3′, [symbol] , [symbol] , [symbol] , and [symbol] are as described herein.

In one aspect, the present disclosure provides compounds, or a stereoisomer, tautomer, prodrug, or salt thereof, of Formula (VIII):

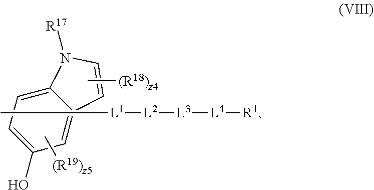

wherein L¹, L², L³, L⁴, and R¹ are as described herein;
R¹⁷, R¹⁸, and R¹⁹ are each independently hydrogen, optionally substituted alkyl, optionally substituted heteroalkyl, optionally substituted cycloalkyl, optionally substituted heterocycloalkyl, optionally substituted aryl, or optionally substituted heteroaryl;
z4 is 0, 1, or 2; and
z5 is 0, 1, 2, or 3.

In one aspect, the present disclosure provides compounds, or a stereoisomer, tautomer, prodrug, or salt thereof, of Formula (VIII-a):

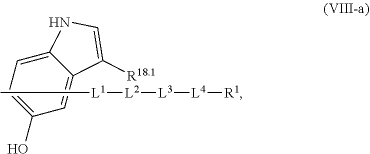

wherein L¹, L², L³, L⁴, and R¹ are as described herein; and R^{18.1} is hydrogen, optionally substituted alkyl or optionally substituted heteroalkyl.

In one aspect, the present disclosure provides compounds, or a stereoisomer, tautomer, prodrug, or salt thereof, of Formula (VIII-a-1):

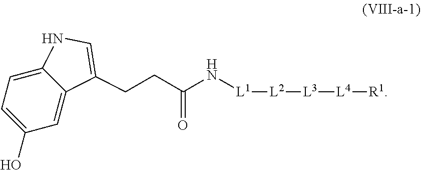

wherein L¹, L², L³, L⁴, and R¹ are as described herein.

In one aspect, the present disclosure provides compounds, or a stereoisomer, tautomer, prodrug, or salt thereof, of Formula (IX):

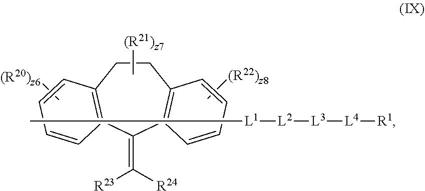

wherein L¹, L², L³, L⁴, and R¹ are as described herein;
R²⁰ is hydrogen, halogen, —CN, —N₃, —SOₙ₂₀R^{1A}, —SOᵥ₂₀NR^{20B}R^{20C}, —NHNR^{20B}R^{20C}, —ONR^{20B}R^{20C}, —NHC(O)NHNR^{20B}R^{20C}, —NHC(O)NR^{20B}R^{20C}, —N(O)ₘ₂₀, —NR^{20B}R^{20C}, —C(O)R^{20D}, —C(O)OR^{20D}, —C(O)NR^{20B}R^{20C}, —OR^{20A}, —NR^{20B}SO₂R^{20A}, —NR^{20B}C(O)R^{20D};
—NR^{20B}C(O)OR^{20D}, —NR^{20B}OR^{20D}, optionally substituted alkyl, optionally substituted heteroalkyl, optionally substituted cycloalkyl, optionally substituted heterocycloalkyl, optionally substituted aryl, or optionally substituted heteroaryl;
R²¹ is hydrogen, halogen, —CN, —N₃, —SOₙ₂₁R^{1A}, —SOᵥ₂₁NR^{21B}R^{21C}, —NHNR^{21B}R^{21C}, —ONR^{21B}R^{21C}, —NHC(O)NHNR^{21B}R^{21C}, —NHC(O)NR^{21B}R^{21C}, —N(O)ₘ₂₁, —NR^{21B}R^{21C}, —C(O)R^{21D}, —C(O)OR^{21D}, —C(O)NR^{21B}R^{21C}, —OR^{21A}, —NR^{21B}SO₂R^{21A}, —NR^{21B}C(O)R^{21D};
—NR^{21B}C(O)OR^{21D}, —NR^{21B}OR^{21D}, optionally substituted alkyl, optionally substituted heteroalkyl, optionally substituted cycloalkyl, optionally substituted heterocycloalkyl, optionally substituted aryl, or optionally substituted heteroaryl;
R²² and R²³ are each independently hydrogen, optionally substituted alkyl, optionally substituted heteroalkyl, optionally substituted cycloalkyl, optionally substituted heterocycloalkyl, optionally substituted aryl, or optionally substituted heteroaryl;
R²⁴ is hydrogen, halogen, —CN, —N₃, —SOₙ₂₄R^{24A}, —SOᵥ₂₄NR^{24B}R^{24C}, —NHNR^{24B}R^{24C}, —ONR^{24B}R^{24C}, —NHC(O)NHNR^{24B}R^{24C}, —NHC(O)NR^{24B}R^{24C}, —N(O)ₘ₂₄, —NR^{24B}R^{24C}, —C(O)R^{24D}, —C(O)OR^{24D}, —C(O)NR^{24B}R^{24C}, —OR^{24A}, —NR^{24B}SO₂R^{24A}, —NR^{24B}C(O)R^{24D};
—NR^{24B}C(O)OR^{24D}, —NR^{24B}OR^{24D}, optionally substituted alkyl, optionally substituted heteroalkyl, optionally substituted cycloalkyl, optionally substituted heterocycloalkyl, optionally substituted aryl, or optionally substituted heteroaryl;
R^{20A}, R^{20B}, R^{20C}, R^{20D}, R^{21A}, R^{21B}, R^{21C}, R^{21D}, R^{24A}, R^{24B}, R^{24C}, and R^{24D} are each independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{20B}, R^{20C}, R^{21B}, R^{21C}, R^{24B}, R^{24C}, R^{24B}, and R₂^{4C} substituents bonded to the same nitrogen atom may optionally be joined to form a substituted or unsubstituted heterocycloalkyl or substituted or unsubstituted heteroaryl;
n21, n22, n24, z6 and z8 are each independently 0, 1, 2, 3, or 4;
v20, v21, v24, m20, m21, and m24 are each independently 1 or 2; and
z7 is 0, 1, or 2.

In one aspect, the present disclosure provides compounds, or a stereoisomer, tautomer, prodrug, or salt thereof, of Formula (IX-a):

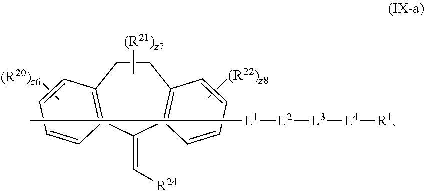

wherein L¹, L², L³, L⁴, R¹, R²⁰, R²¹, R²², R²⁴, z6, 7, and z8 are as described herein.

In one aspect, the present disclosure provides compounds, or a stereoisomer, tautomer, prodrug, or salt thereof, of Formula (IX-a-1):

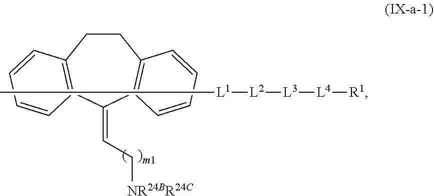

wherein L¹, L², L³, L⁴, R¹, R^{24B}, R^{24C}, and m1 are as described herein.

In one aspect, the present disclosure provides compounds, or a stereoisomer, tautomer, prodrug, or salt thereof, of Formula (IX-a-2):

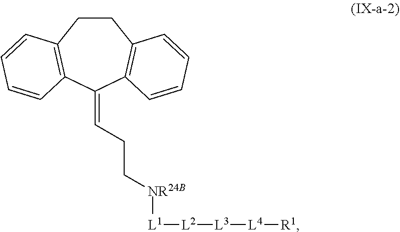

wherein L¹, L², L³, L⁴, R¹, R^{24B}, and R^{24C} are as described herein.

In certain embodiments, R¹⁶, R²⁰, R²¹, R²⁴ are each independently —OH.

In one aspect, the present disclosure provides compounds, or a stereoisomer, tautomer, prodrug, or salt thereof, of Formula (X):

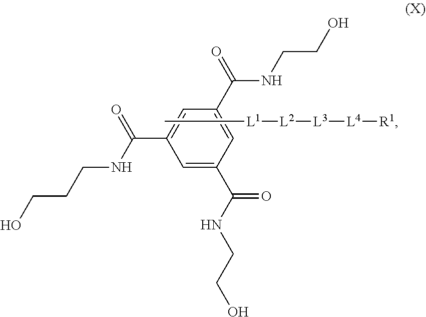

wherein L¹, L², L³, L⁴, and R¹ are as described herein.

In one aspect, the present disclosure provides compounds, or a stereoisomer, tautomer, prodrug, or salt thereof, of Formula (X-a):

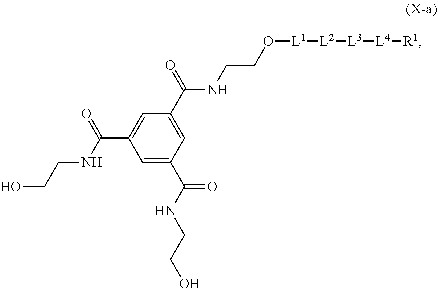

wherein L¹, L², L³, L⁴, and R¹ are as described herein.

In one aspect, the present disclosure provides compounds, or a stereoisomer, tautomer, prodrug, or salt thereof, of Formula (XI):

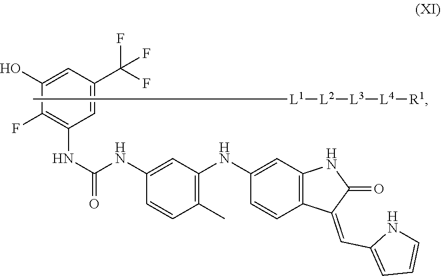

wherein L¹, L², L³, L⁴, and R¹ are as described herein.

In one aspect, the present disclosure provides compounds, or a stereoisomer, tautomer, prodrug, or salt thereof, of Formula (XI-a):

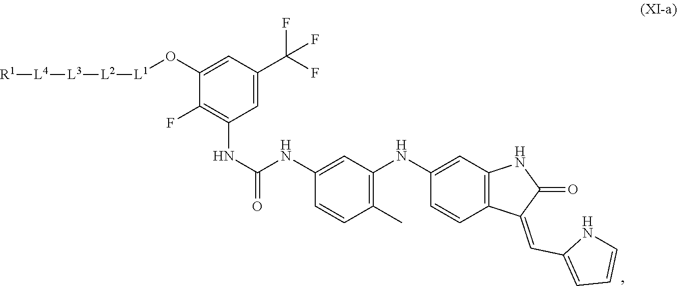

wherein L¹, L², L³, L⁴, and R¹ are as described herein.

In one aspect, the present disclosure provides precursor compounds, or a stereoisomer, tautomer, or salt thereof, of Formulae (A)-(F):

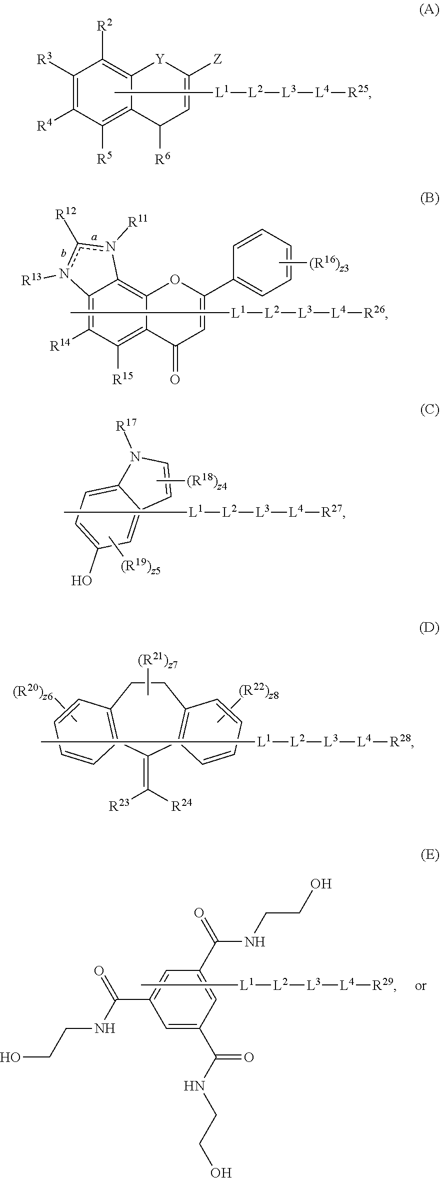

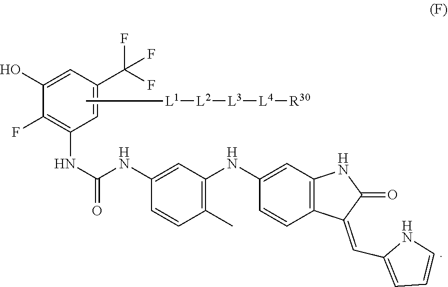

wherein L¹, L², L³, L⁴, R¹, R², R³, R⁴, R⁵, R⁶, R⁷, R⁸, R⁹, R¹⁰, R¹¹, R^{11′}, R¹², R^{12′}, R¹³, R^{13′}, R¹⁴, R^{14′}, R¹⁵, R^{15′}, R¹⁶, R^{16′}, R¹⁷, R¹⁸, R¹⁹, R²⁰, R²¹, R²², R²³, R²⁴, Y, X, z3 z3′, z4, z5, z6, z7, z8, [symbol] , [symbol] , [symbol] , and [symbol] are as described herein; and R²⁵, R²⁶, R²⁷, R²⁸, R²⁹, and R³⁰ are
each independently —N₃ or

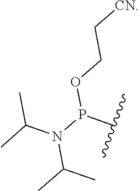 [SMILES: *P(OCCC#N)N(C(C)C)C(C)C]

In certain embodiments, z1 is 1; and z1′ is 0 or 1. In certain embodiments, z1 is 1 and z1′ is 0. In certain embodiments, z1 and z1′ are each independently 1.

In certain embodiments, the TrkB ligand comprises a substituted or unsubstituted aryl, substituted or unsubstituted heteroaryl, substituted or unsubstituted heterocycle, substituted or unsubstituted heterocycloalkyl, or substituted or unsubstituted cycloalkyl. In certain embodiments, the TrkB ligand comprises a substituted or unsubstituted aryl, substituted or unsubstituted heteroaryl, or substituted or unsubstituted heterocycloalkyl. In certain embodiments, the TrkB ligand comprises a substituted or unsubstituted mono- or polycyclic aryl, substituted or unsubstituted mono- or polycyclic heteroaryl, substituted or unsubstituted mono- or polycyclic heterocycloalkyl, or substituted or unsubstituted mono- or polycyclic cycloalkyl. In certain embodiments, the substituted or unsubstituted polycyclic aryl, substituted or unsubstituted polycyclic heteroaryl, substituted or unsubstituted polycyclic heterocycloalkyl or substituted or unsubstituted mono- or polycyclic cycloalkyl are a fused ring system. In certain embodiments, the fused ring system comprises two to four fused rings. In certain embodiments, the fused ring system further comprises an optionally substituted pendant cycloalkyl, optionally substituted pendant heterocycloalkyl, optionally substituted pendant aryl, or an optionally substituted pendant heteroaryl

In certain embodiments, each TrkB ligand is independently:

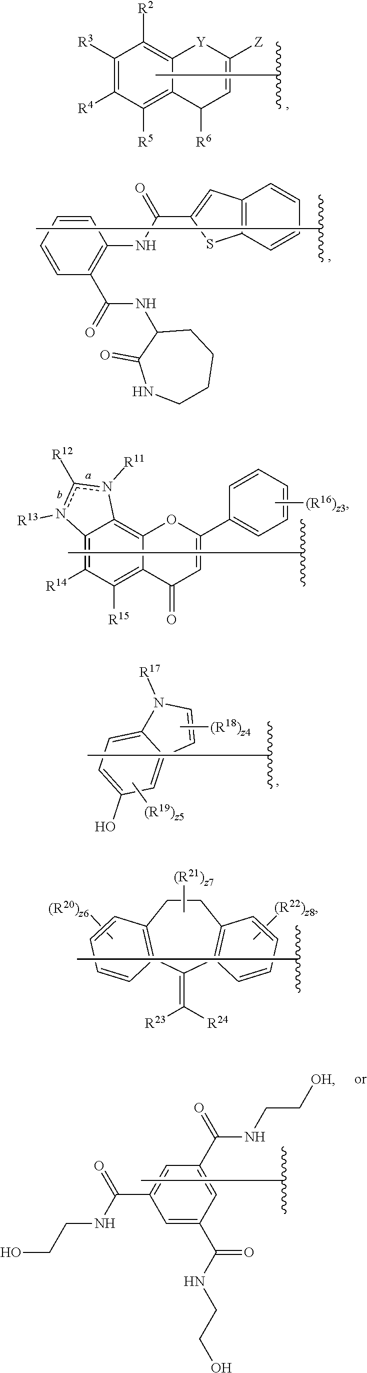

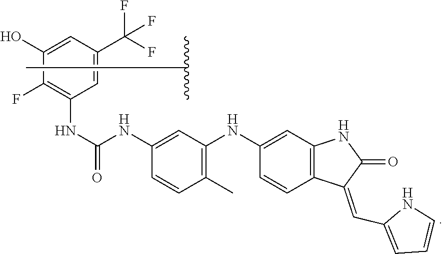

In certain embodiments, each TrkB ligand is independently flavone, tropoflavin, or a derivative thereof.

In certain embodiments, each TrkB ligand is independently 3,7-dihydroxyflavone, 3,7,8,2′-tetrahydroxyflavone, 7,3′-dihydroxyflavone, 7,8,2′-trihydroxyflavone, 7,8,3′-trihydroxyflavone, 7,8,4′-trihydroxyflavone, diosmetin (5,7,3′-trihydroxy-4′-methoxyflavone), 7-hydroxy-4′-methoxyflavone, 8-hydroxy-7-methoxyflavone, eutropoflavin (4′-dimethylamino-7,8-dihydroxyflavone), norwogonin (5,7,8-trihydroxyflavone), R7, R13, tropoflavin (7,8-dihydroxyflavone), quercetin (3,3′,4′,5,7-pentahydroxyflavone), apigenin (4′,5,7-trihydroxyflavone), isocoumarin, gossypetin (3,5,7,8,3′,4′-hexahydroxyflavone), 2-methyl-8-phenylchromeno[7,8-d]imidazol-6 (3H)-one, 8-phenylchromeno[7,8-d]imidazol-6 (3H)-one, 4-oxo-2-phenyl-4H-chromene-7,8-diyl diacetate, ANA-12, an anti-TrkB antibody, or derivatives thereof.

In certain embodiments, R² is —OH, —O-alkyl, or —O-alkenyl, and R³ is —OH, —O-alkyl, or —O-alkenyl. In certain embodiments, R² is —OH. In certain embodiments, R³ is —OH. In certain embodiments, R⁴ is hydrogen. In certain embodiments, R⁵ is hydrogen. In certain embodiments, R⁶ is ═O. In certain embodiments, Y is O. In certain embodiments, Z is optionally substituted aryl. In certain embodiments, Z is optionally substituted phenyl. In certain embodiments, Z is unsubstituted phenyl. In certain embodiments, Z is phenyl substituted with one or more —OH and/or —O-alkyl.

In certain embodiments, the TrkB ligand is ANA-12, or a prodrug or derivative thereof.

In certain embodiments, R¹⁶ is —NR^{16B}R^{16C}; and R^{16B} and R^{16C} are each independently hydrogen or optionally substituted alkyl. In certain embodiments, R^{16B} is hydrogen; and R^{16C} is C₁-C₃ alkyl. In certain embodiments, R^{16C} is —CH₃.

In certain embodiments, R¹² is hydrogen or optionally substituted alkyl. In certain embodiments, R¹² is hydrogen or C₁-C₃ alkyl. In certain embodiments, R¹² is hydrogen or —CH₃.

In certain embodiments, R^{24B} is hydrogen or optionally substituted alkyl. In certain embodiments, R^{24B} is C₁-C₃ alkyl. In certain embodiments, R^{24B} is —CH₃.

In certain embodiments, the TrkB ligand is a TrkB selective ligand or a TrkB non-selective ligand.

In certain embodiments, L¹, L², L³, L⁴, L^{1′}, L^{2′}, L^{3′}, and L^{4′} are each independently absent, a bond, an optionally substituted alkyl linker, an optionally substituted polyethylene glycol (PEG) linker, an optionally substituted heteroalkyl linker, or an optionally substituted heteroaryl linker.

In certain embodiments, L¹ and L^{1′} are each independently an optionally substituted heteroaryl linker.

In certain embodiments, L¹ and L^{1′} are each independently an optionally substituted unsaturated heteroaryl, an optionally substituted heteroaryl or an optionally substituted saturated or partially unsaturated heterocycloalkyl linker.

In certain embodiments, L¹ and L^{1′} each independently comprise the structure

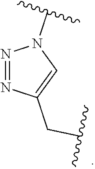 [SMILES: *Cc1cn(*)nn1]

In certain embodiments, L¹ and L^{1′} are each independently an optionally substituted heteroalkyl linker. In certain embodiments, the optionally substituted heteroalkyl linker is an optionally substituted heteroalkyl or optionally substituted C₁-C₁₀ alkyl chain in which one or more carbon atoms are replaced with O, N, or S.

In certain embodiments, L¹ and L^{1′} each independently comprise the structure

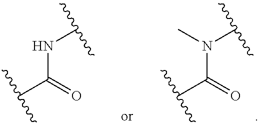

In certain embodiments, L¹ and L^{1′} each independently comprise the structure

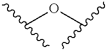 [SMILES: *O*]

or —N(CH₃)—. In certain embodiments, L² and L^{2′} are each independently an optionally substituted PEG linker.

In certain embodiments, the PEG linker is five PEG units in length. In certain embodiments, the PEG linker is four PEG units in length. In certain embodiments, the PEG linker is three PEG units in length.

In certain embodiments, L² and L^{2′} are each independently an optionally substituted alkyl linker. In certain embodiments, L² and L^{2′} are each independently an optionally substituted C₁₋₂₀ alkyl linker. In certain embodiments, L² and L^{2′} are each independently an optionally substituted C₈ alkyl linker. In certain embodiments, L³ and L^{3′} are each independently is an optionally substituted heteroaryl linker.

In certain embodiments, L³ and L^{3′} are each independently an optionally substituted partially unsaturated heteroaryl linker, an optionally substituted heteroaryl or an optionally substituted saturated or partially unsaturated heterocycloalkyl linker.

In certain embodiments, L³ and L^{3′} each independently comprise the structure

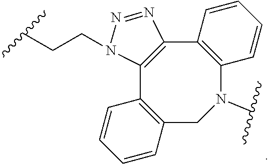 [SMILES: *CCn1nnc2c1-c1ccccc1CN(*)c1ccccc1-2]

In certain embodiments, L⁴ and L^{4′} are each independently an optionally substituted heteroalkyl linker. In certain embodiments, the heteroalkyl linker is substituted with one or more ═O substituents.

In certain embodiments, the heteroalkyl linker comprises two substituents joined together to form an optionally substituted carbocyclyl ring.

In certain embodiments, L⁴ and L^{4′} each independently comprise the structure

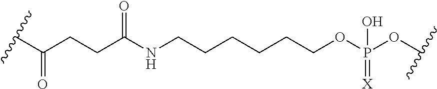

or a salt thereof, wherein X is O or S.

In certain embodiments, L⁴ and L^{4′} each independently comprise the structure:

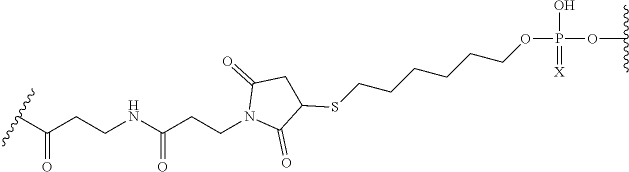

or a salt thereof, wherein X is O or S.

In certain embodiments, L¹-L²-L³-L⁴; or L^{1′}-L^{2′}-L^{3′}-L^{4′} each independently comprise the structure:

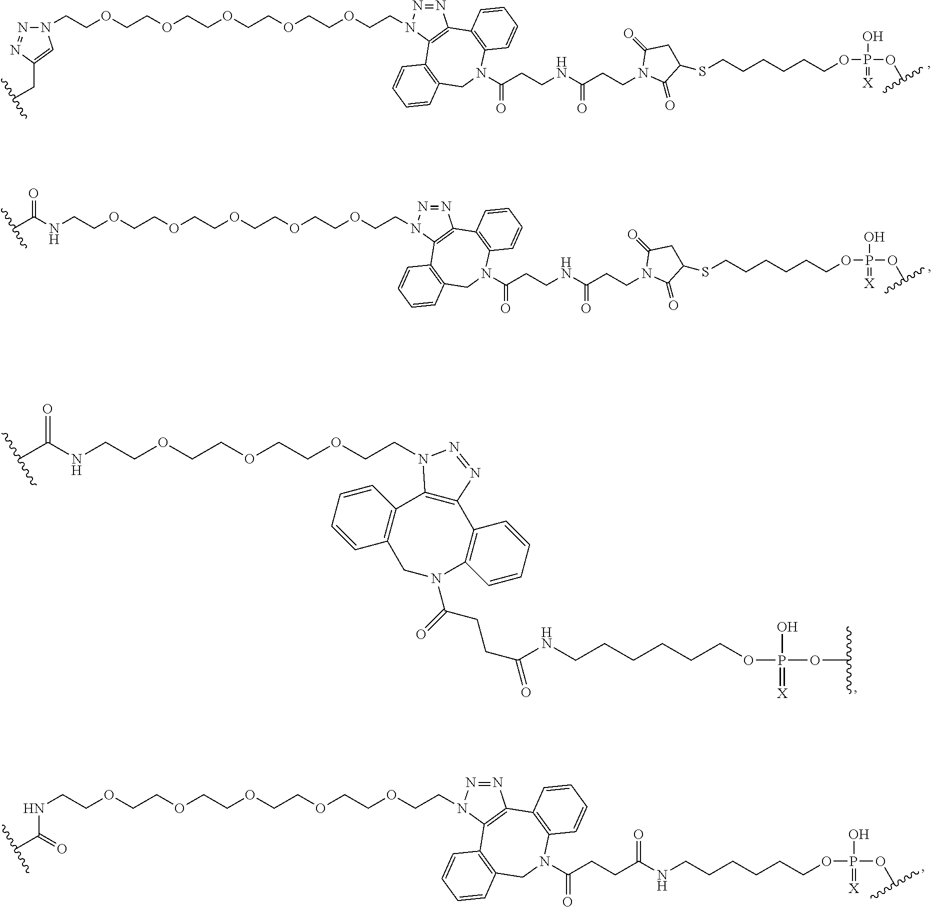

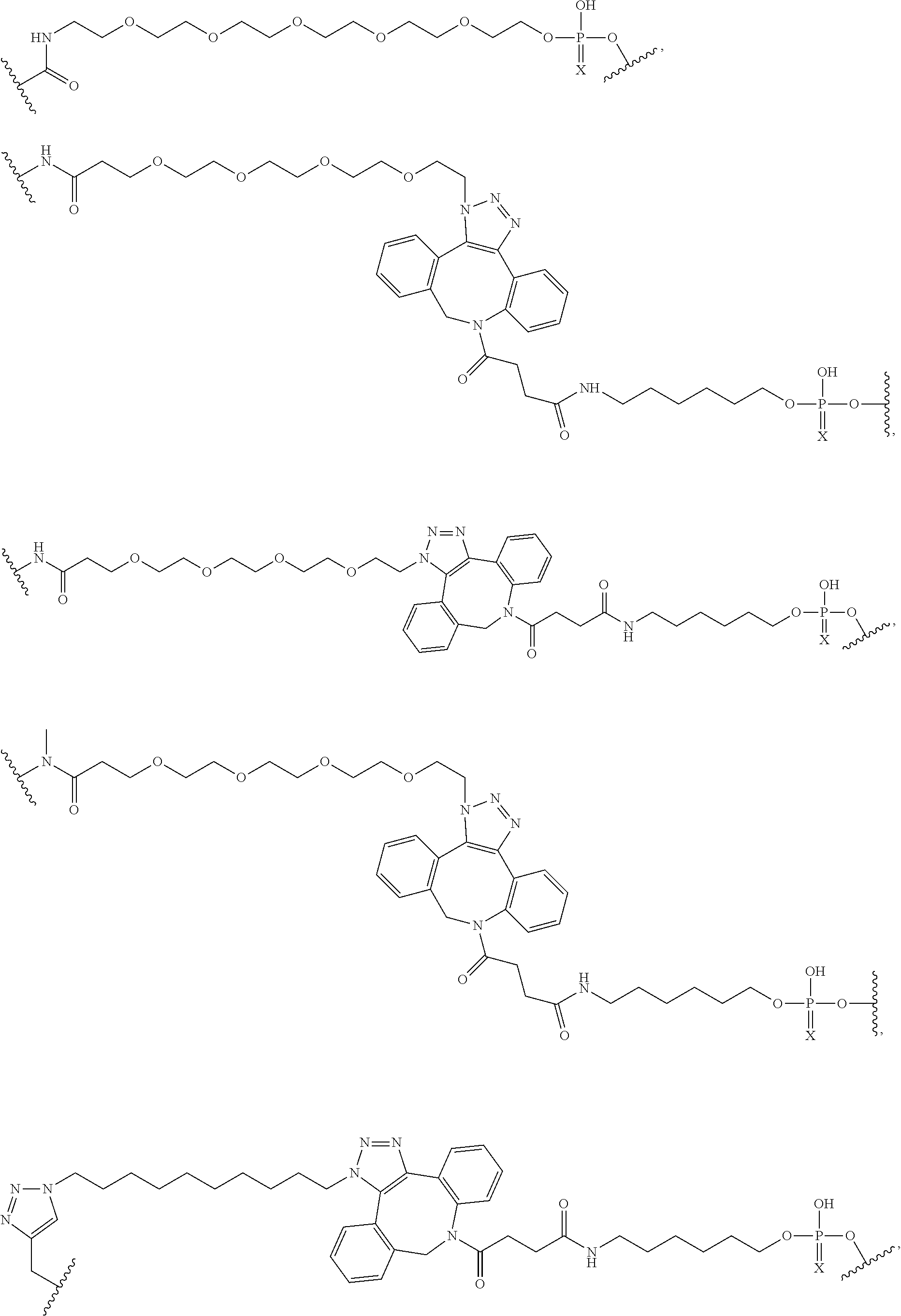

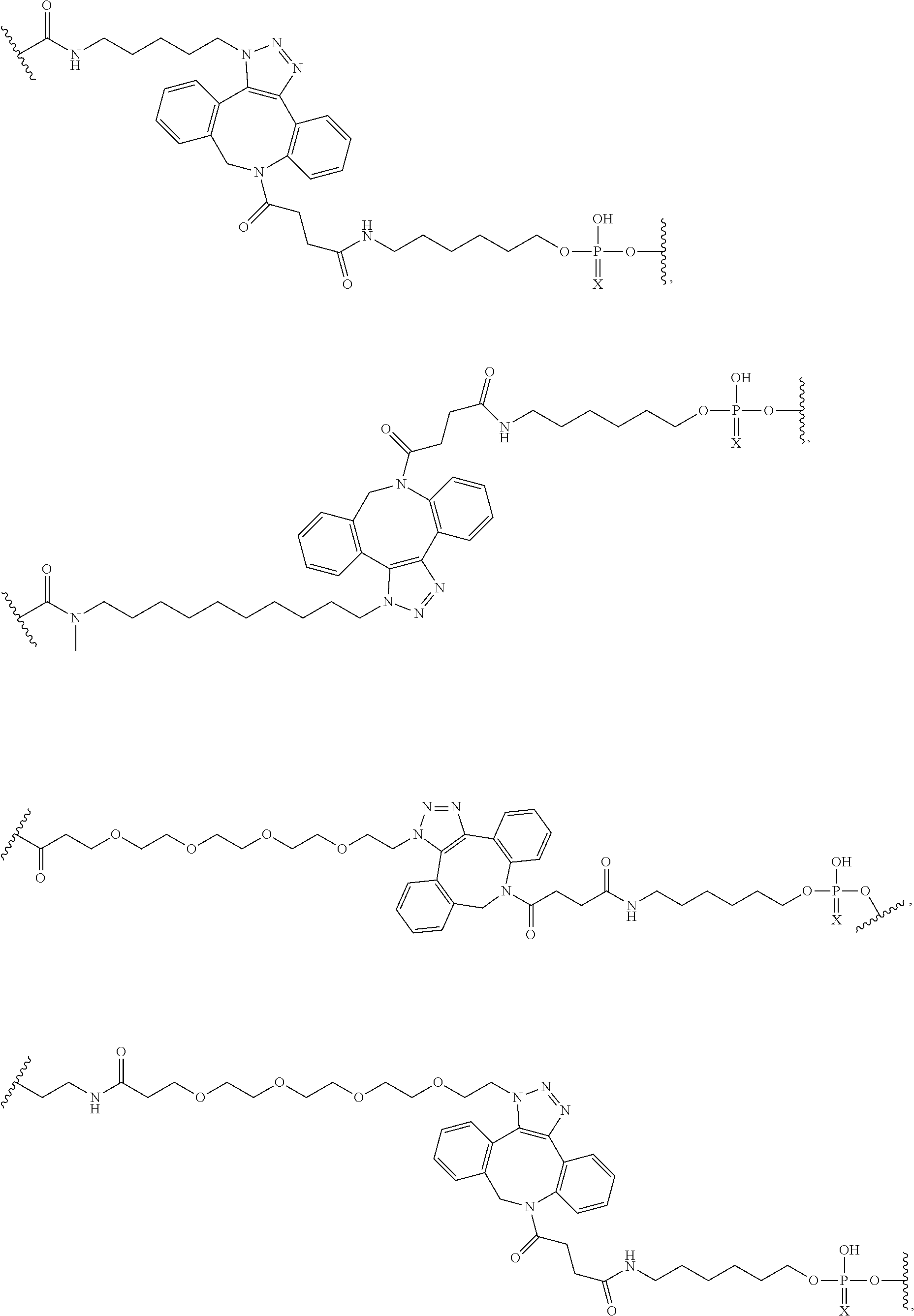

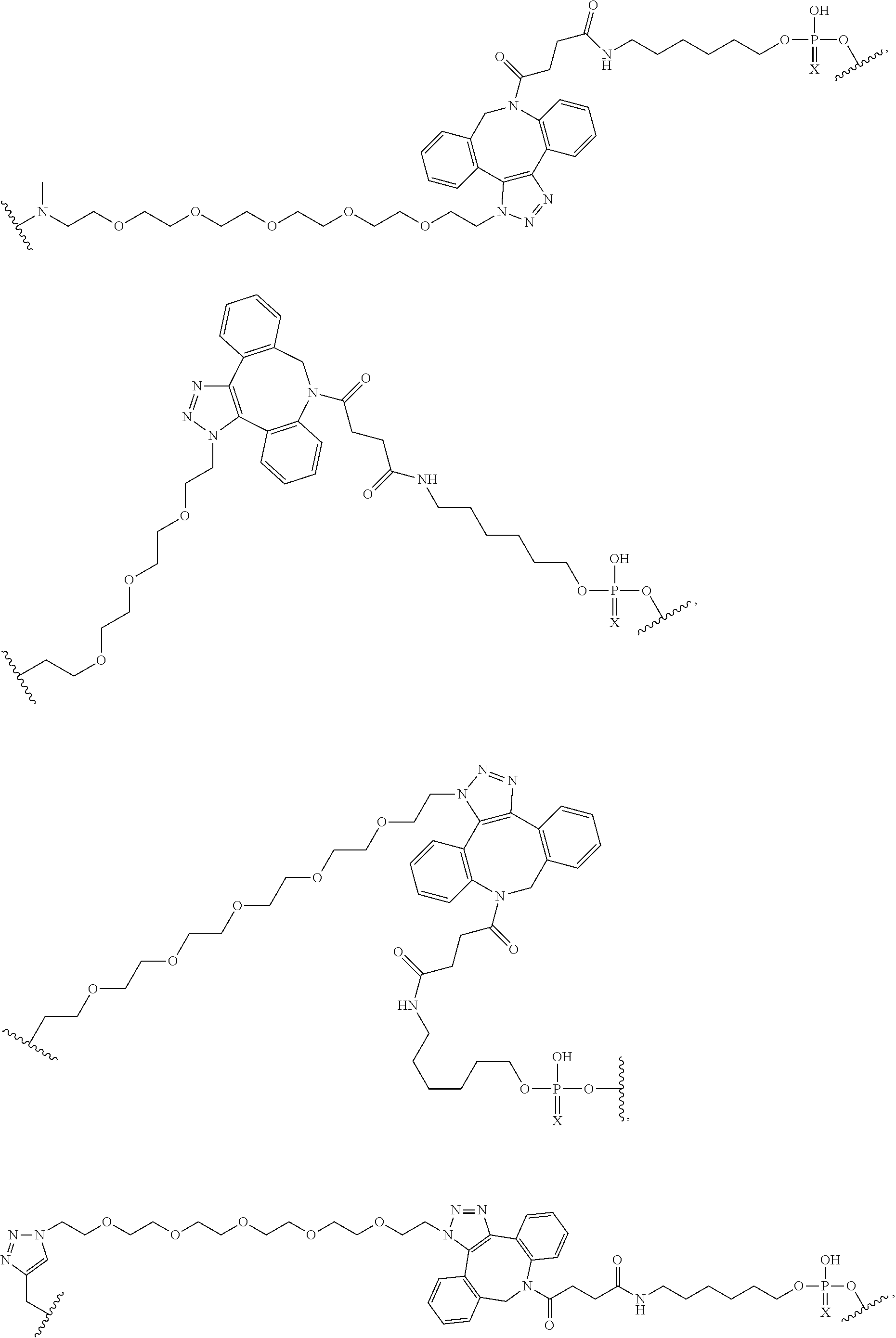

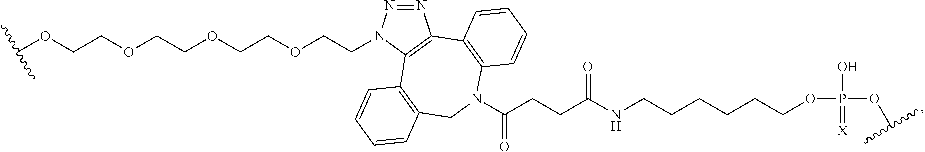

or a salt thereof, wherein X is O or S.

In certain embodiments there is provided a compound, or a stereoisomer, tautomer, prodrug, or salt thereof, comprising the structure:

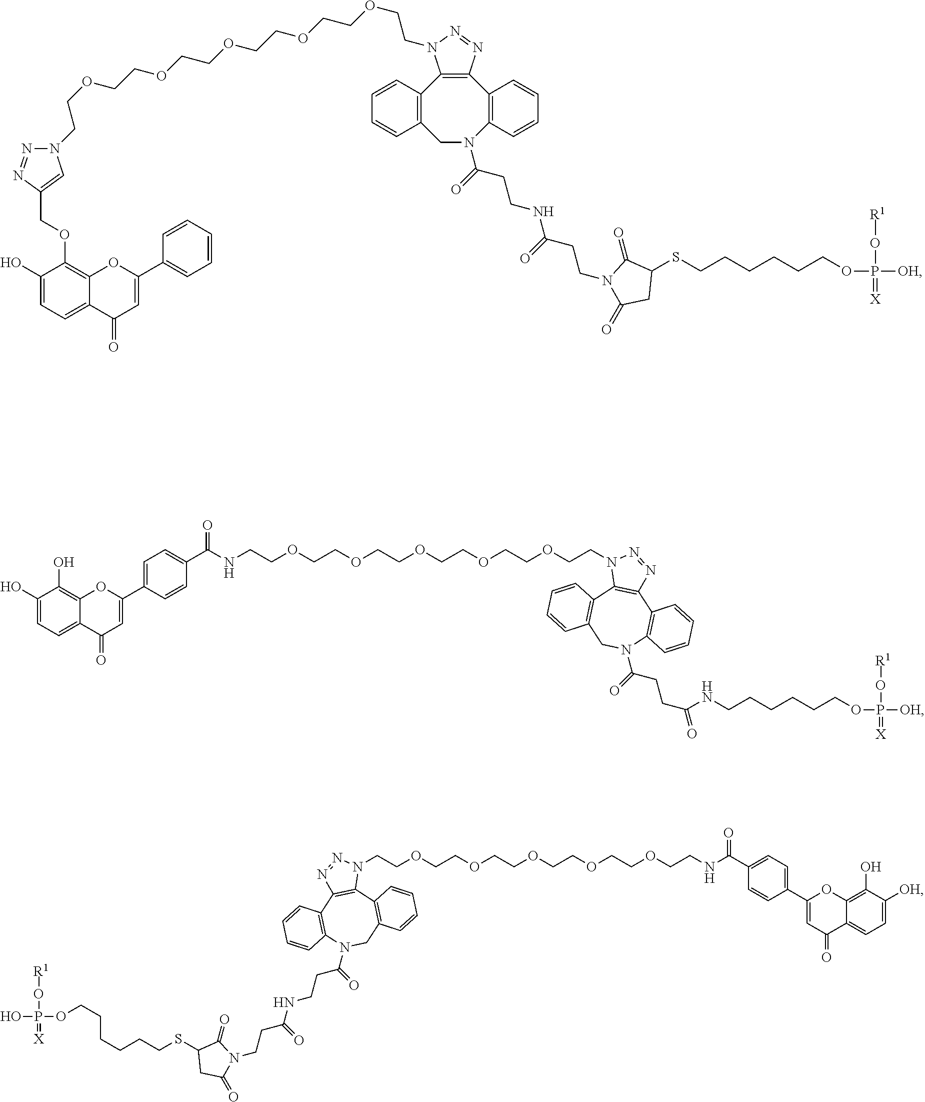

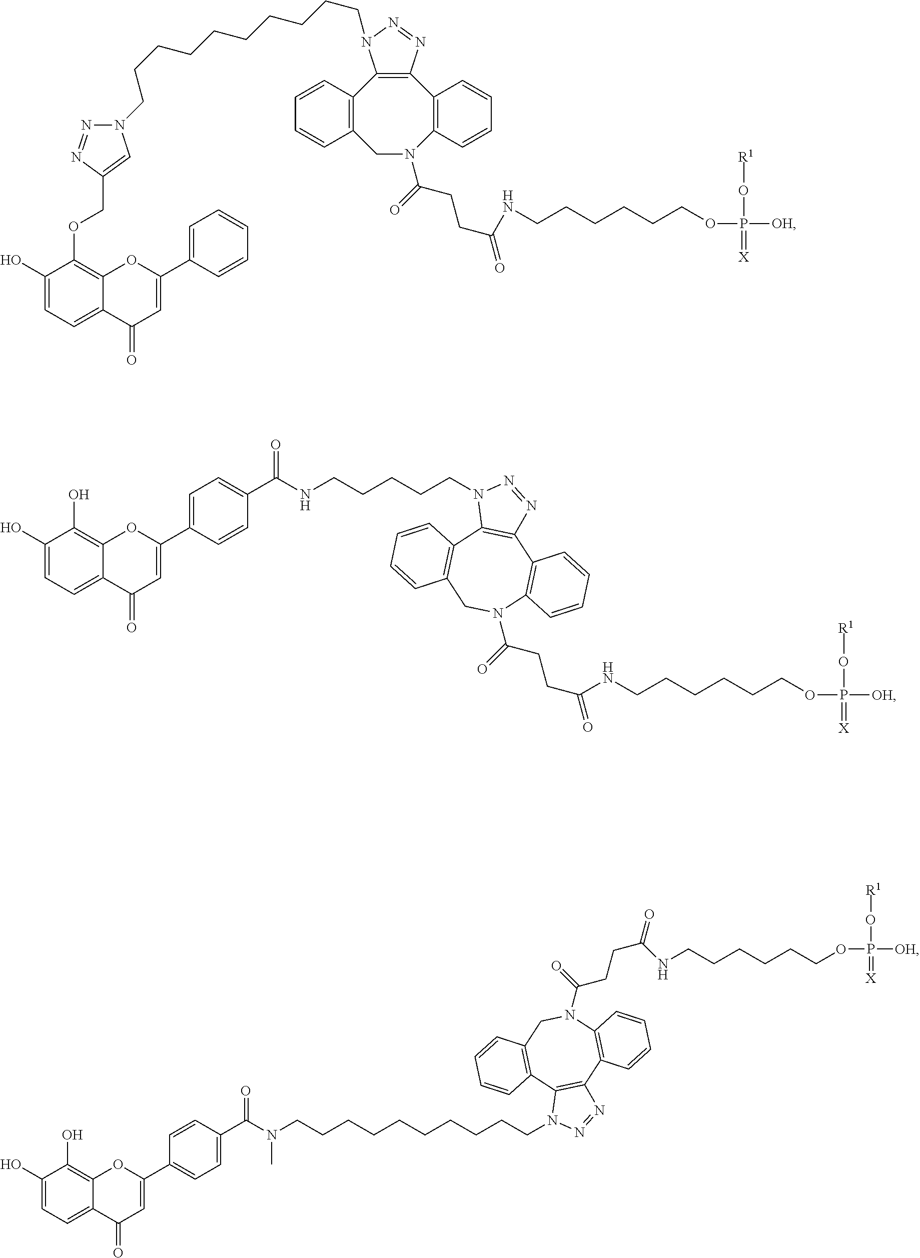

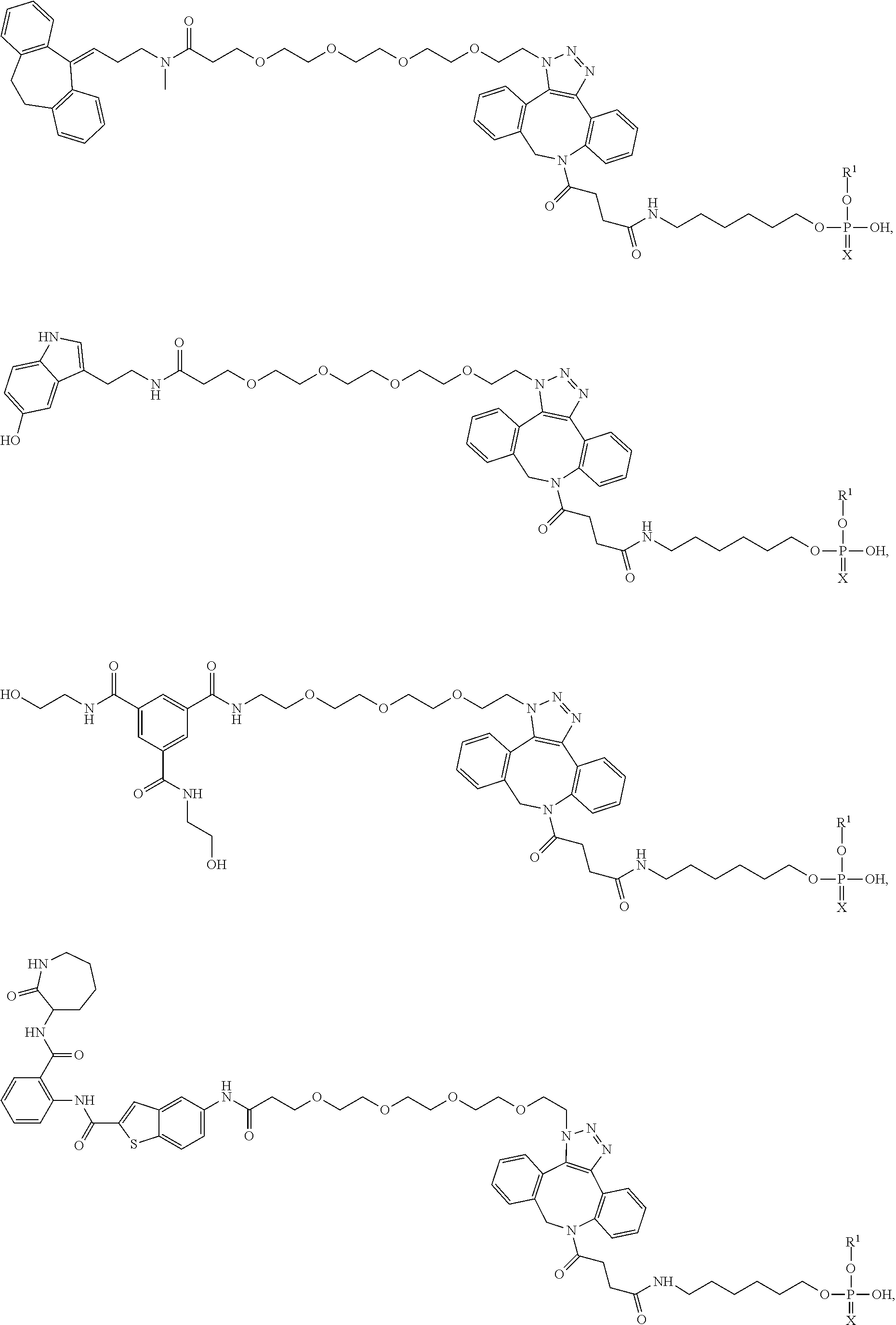

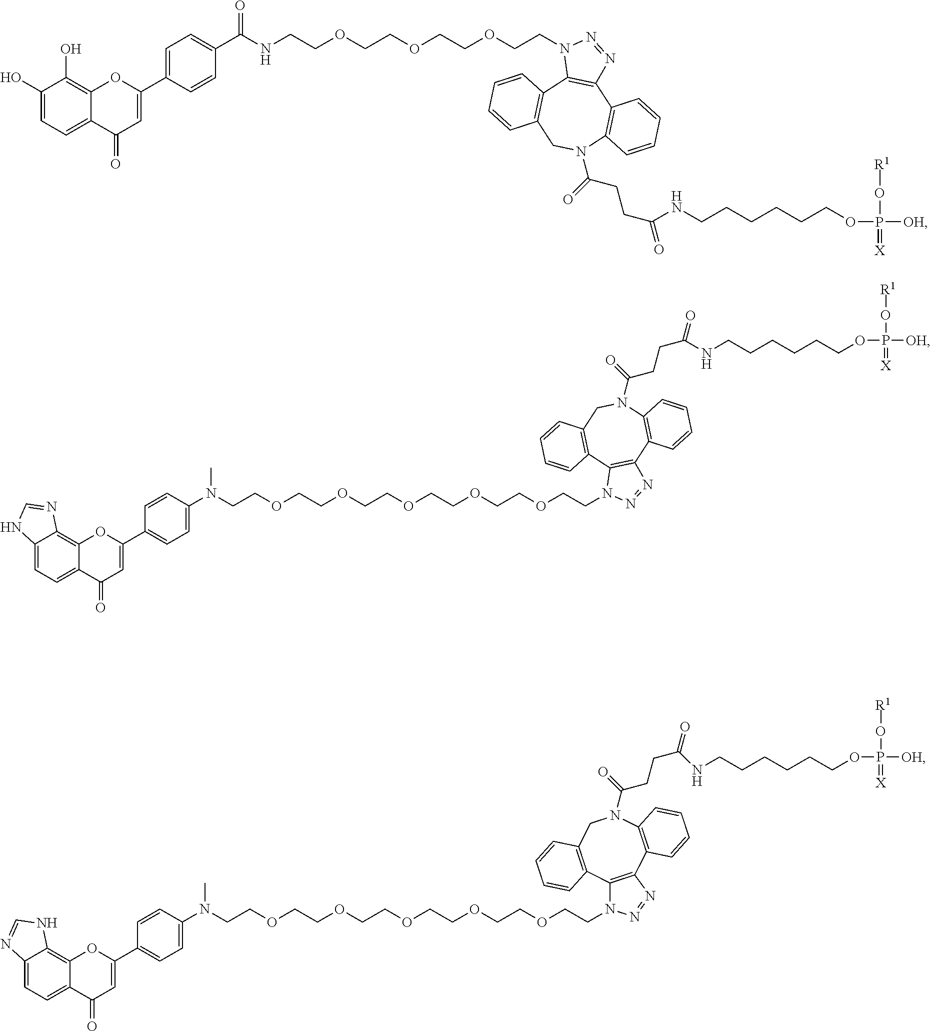

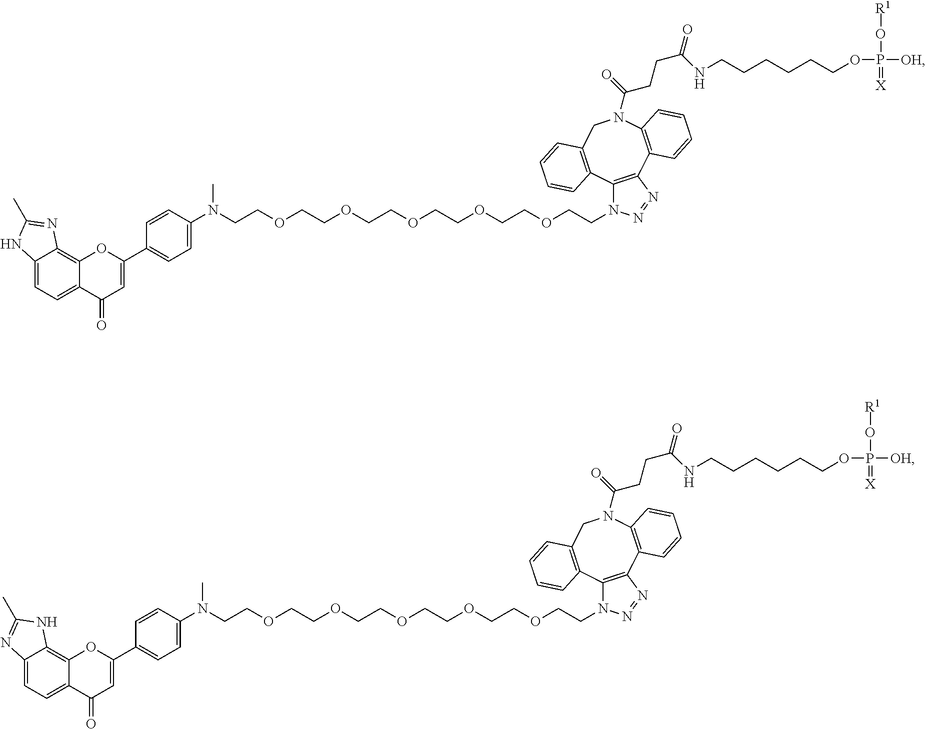

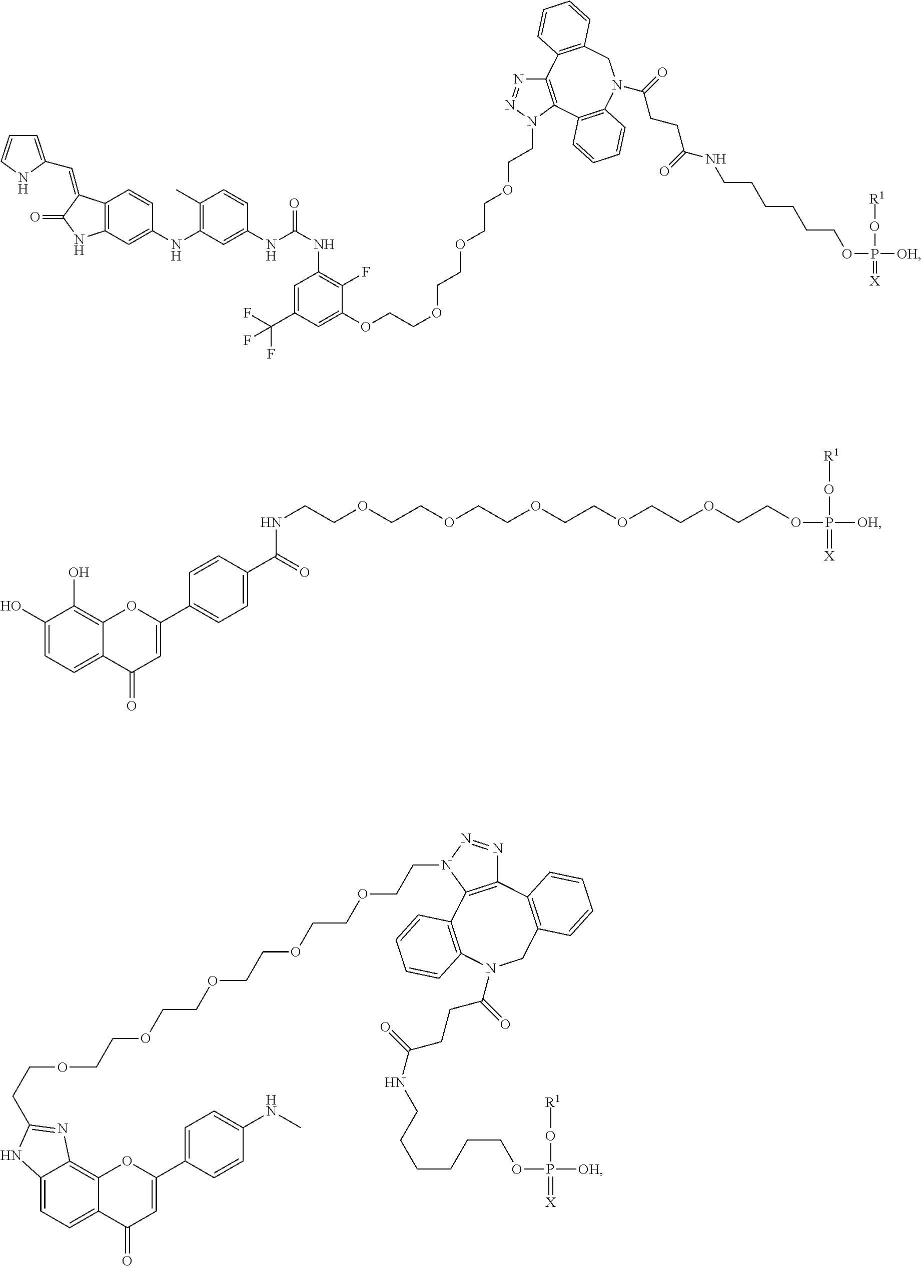

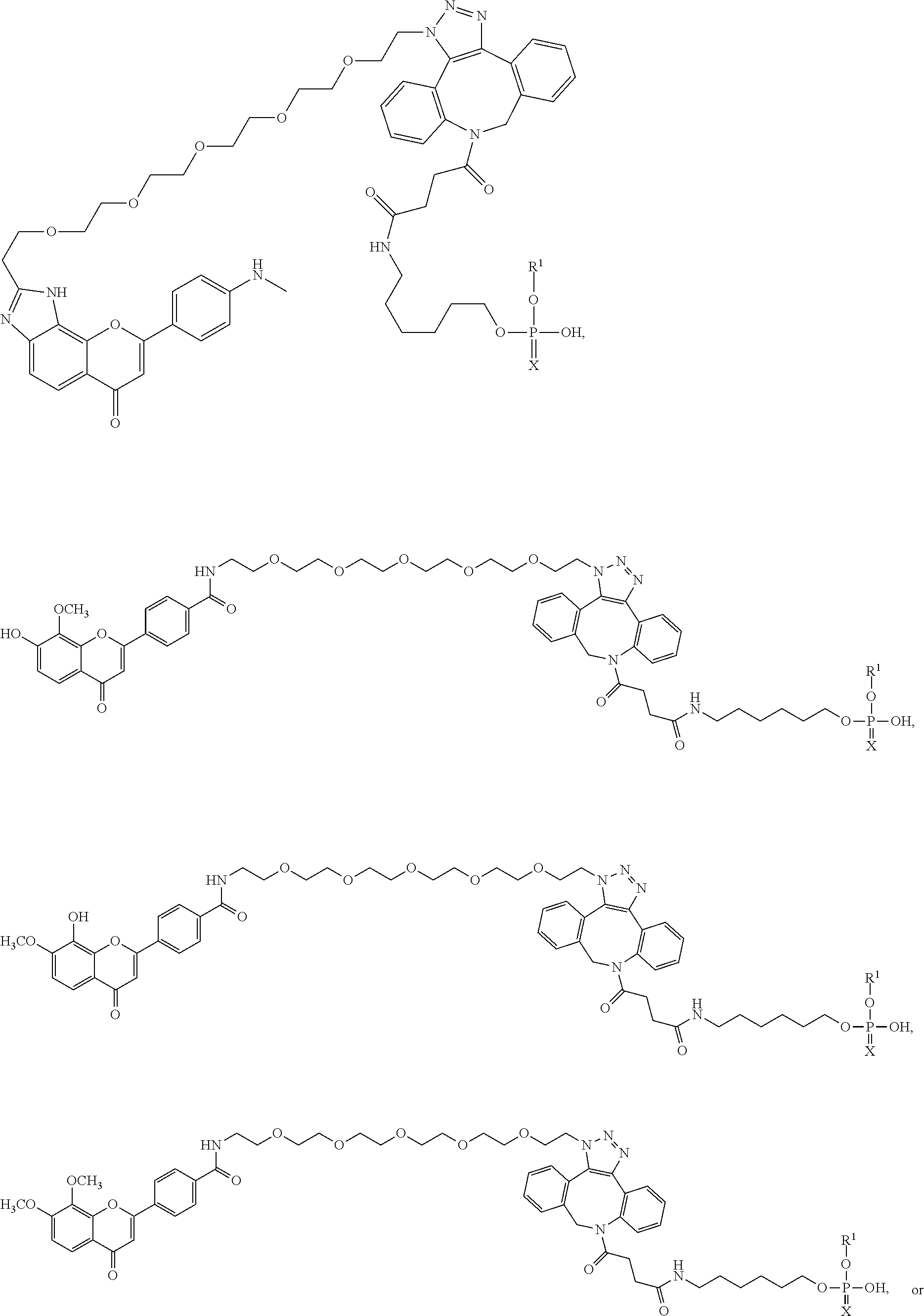

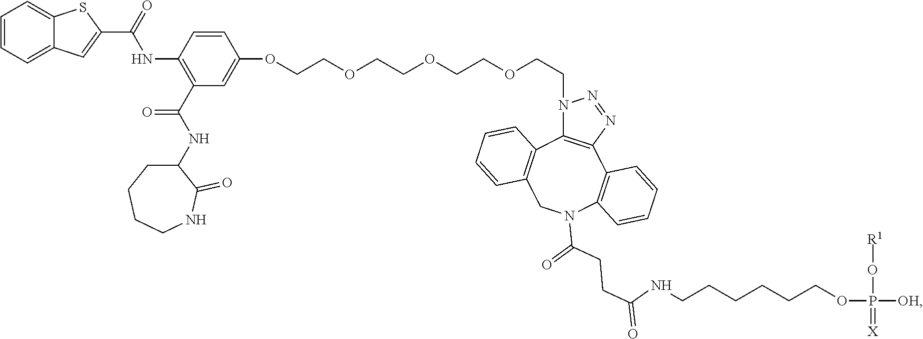

R¹ is an oligonucleotide, a protecting group, a small molecule, a protein, an antibody, or a peptide; and
X is O or S.

In certain embodiments, there is provided a compound, or a stereoisomer, tautomer, prodrug, or salt thereof, comprising the structure:

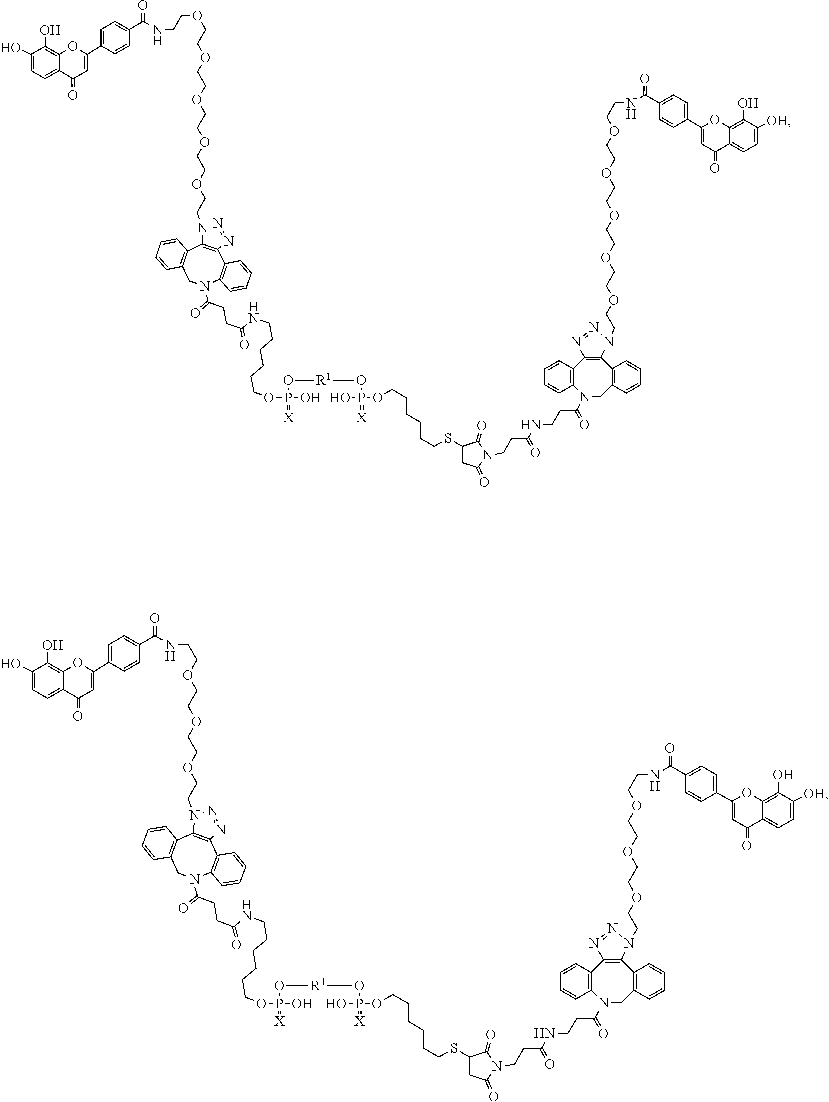

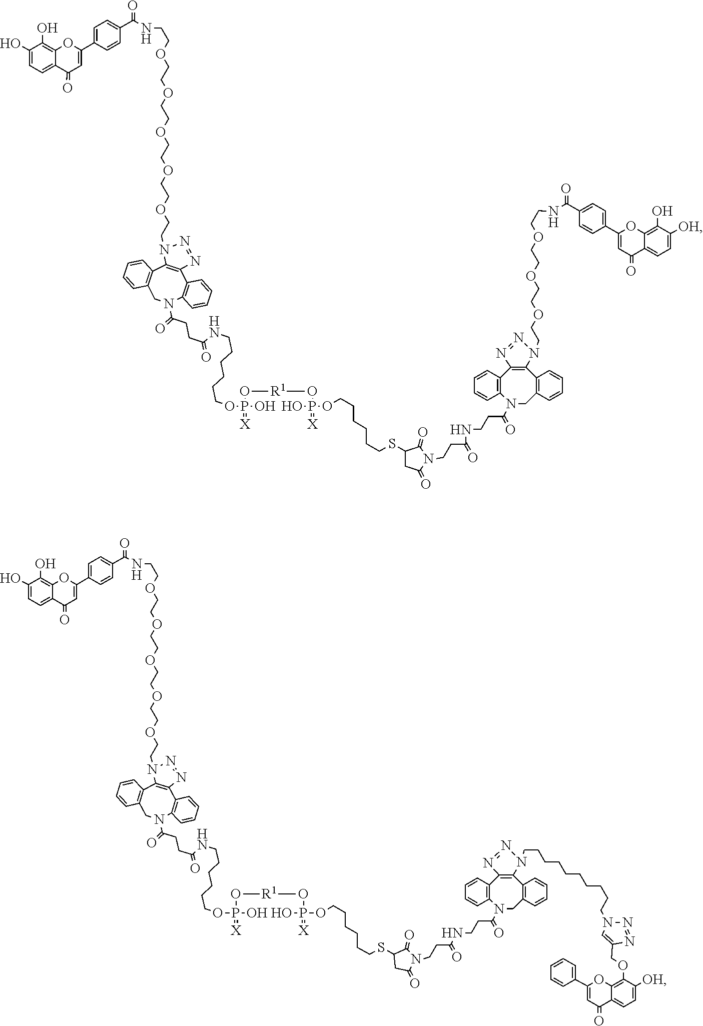

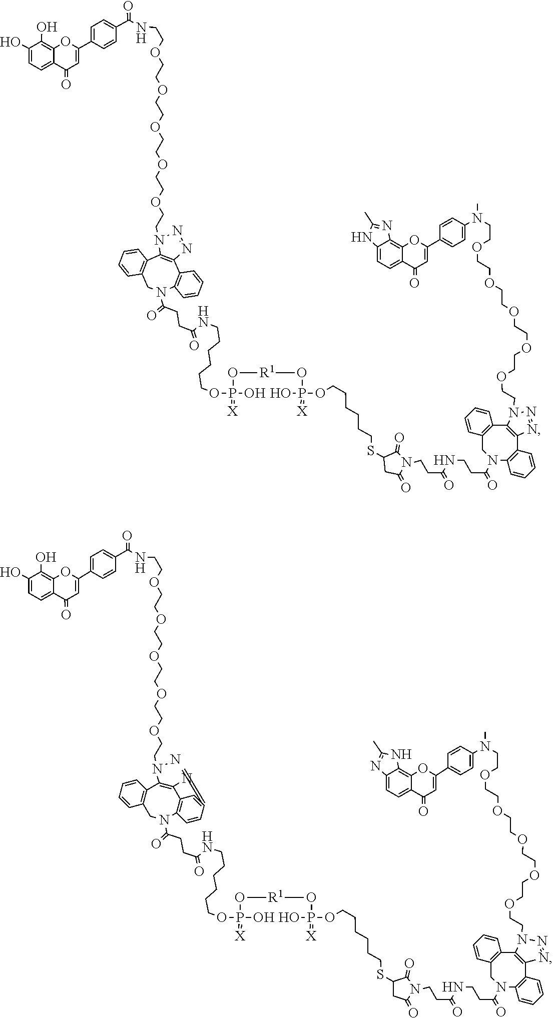

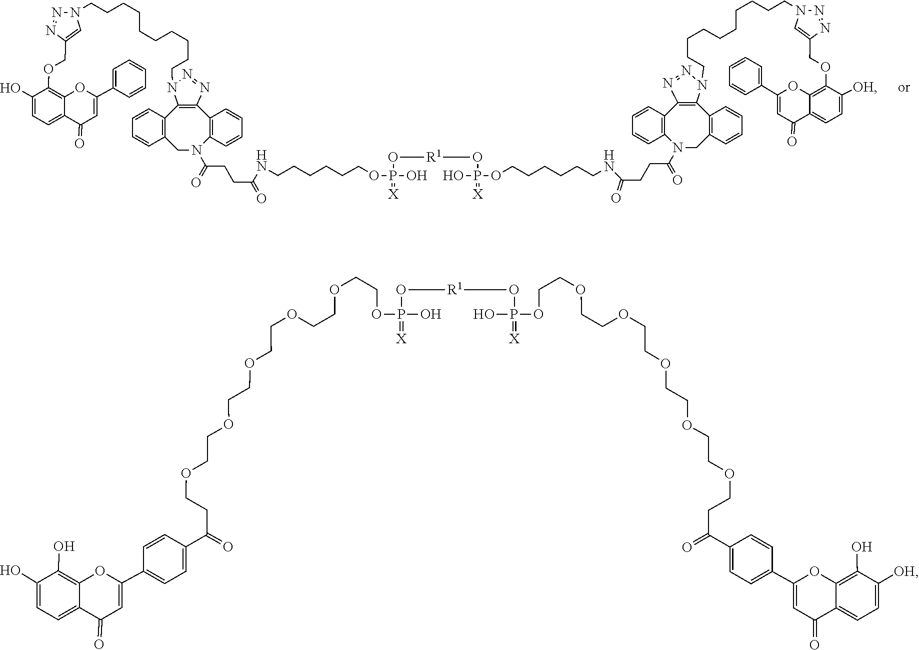

wherein:
R¹ is an oligonucleotide, a protecting group, a small molecule, a protein, an antibody, or a peptide; and
X is O or S.

In certain embodiments, there is provided a compound, or a stereoisomer, tautomer, prodrug, or salt thereof, comprising the structure:

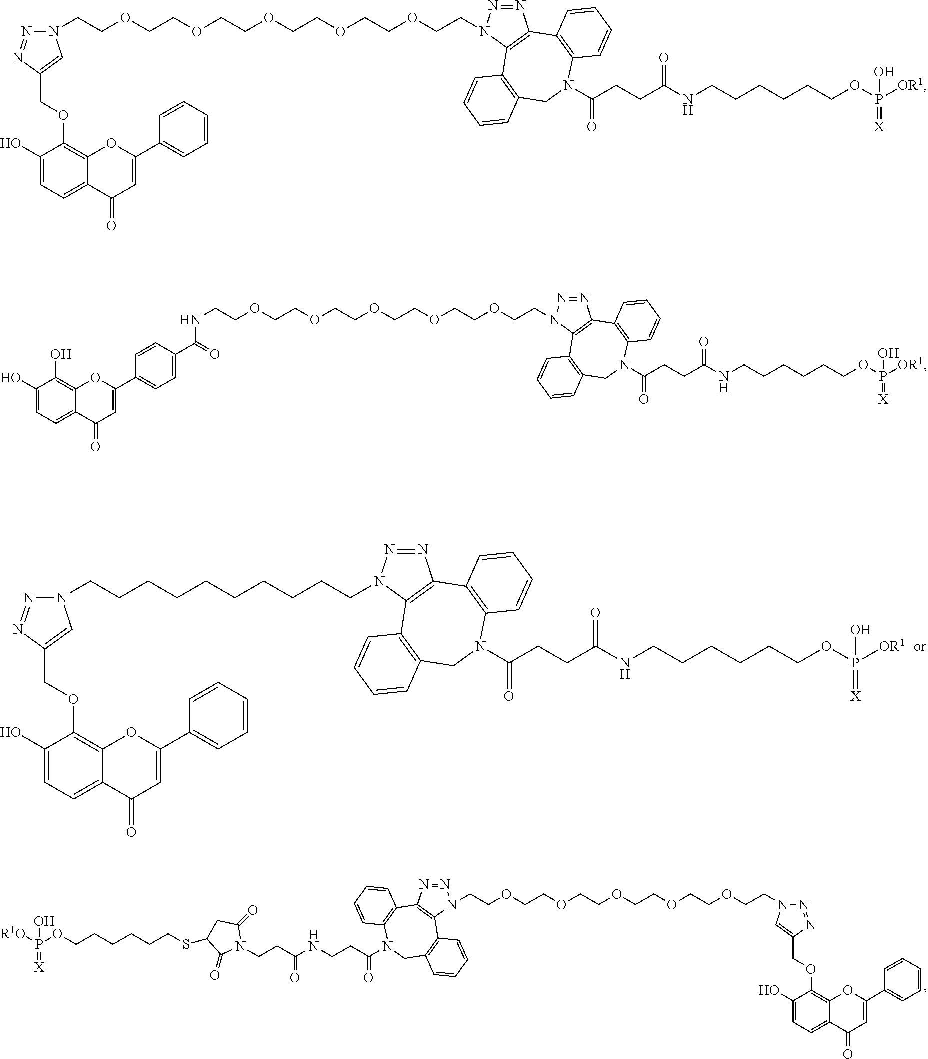

wherein:
R¹ is an oligonucleotide, a protecting group, a small molecule, a protein, an antibody, or a peptide; and
X is O or S.

In certain embodiments, there is provided a compound, or a stereoisomer, tautomer, prodrug, or salt thereof, comprising the structure:

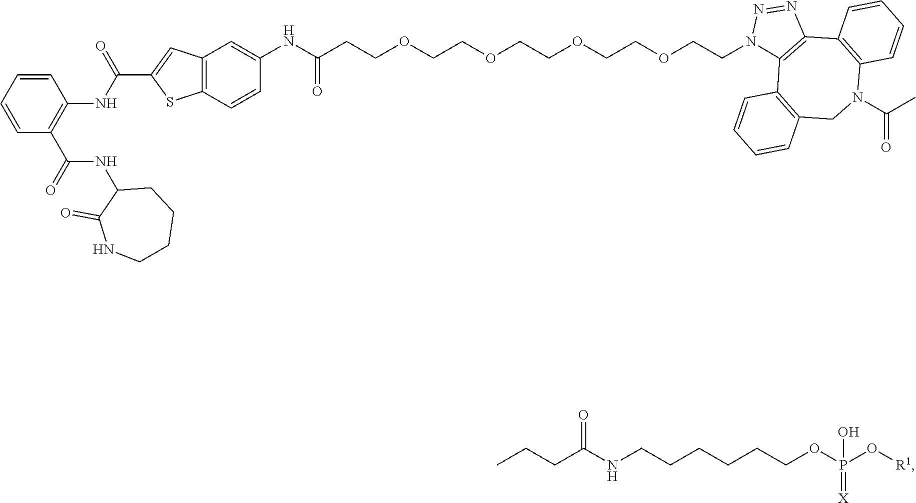

wherein:
R¹ is an oligonucleotide, a protecting group, a small molecule, a protein, an antibody, or a peptide; and
X is O or S.

In certain embodiments, X is O. In certain embodiments, X is S.

In certain embodiments, R¹ comprises an oligonucleotide. In certain embodiments, the oligonucleotide is attached at its 5′ end. In certain embodiments, the oligonucleotide is attached at its 3′ end. In certain embodiments, the oligonucleotide is attached at an internal position on the oligonucleotide. In certain embodiments, the internal position is an internucleoside linkage.

In certain embodiments, R¹ comprises an oligonucleotide conjugated to one or more additional TrkB ligands. In certain embodiments, the oligonucleotide is conjugated to two, three, four, five, or more than five additional TrkB ligands.

In certain embodiments, the additional TrkB ligands are conjugated to the oligonucleotide at the 5′ end of the oligonucleotide, the 3′ end of the oligonucleotide, the 5′- and 3′ ends of the oligonucleotide, one or more internal positions on the oligonucleotide, or any combination thereof.

In certain embodiments, the oligonucleotide is a modified oligonucleotide.

In certain embodiments, there is provided a compound, or a stereoisomer, tautomer, prodrug, or salt thereof, comprising the structure:

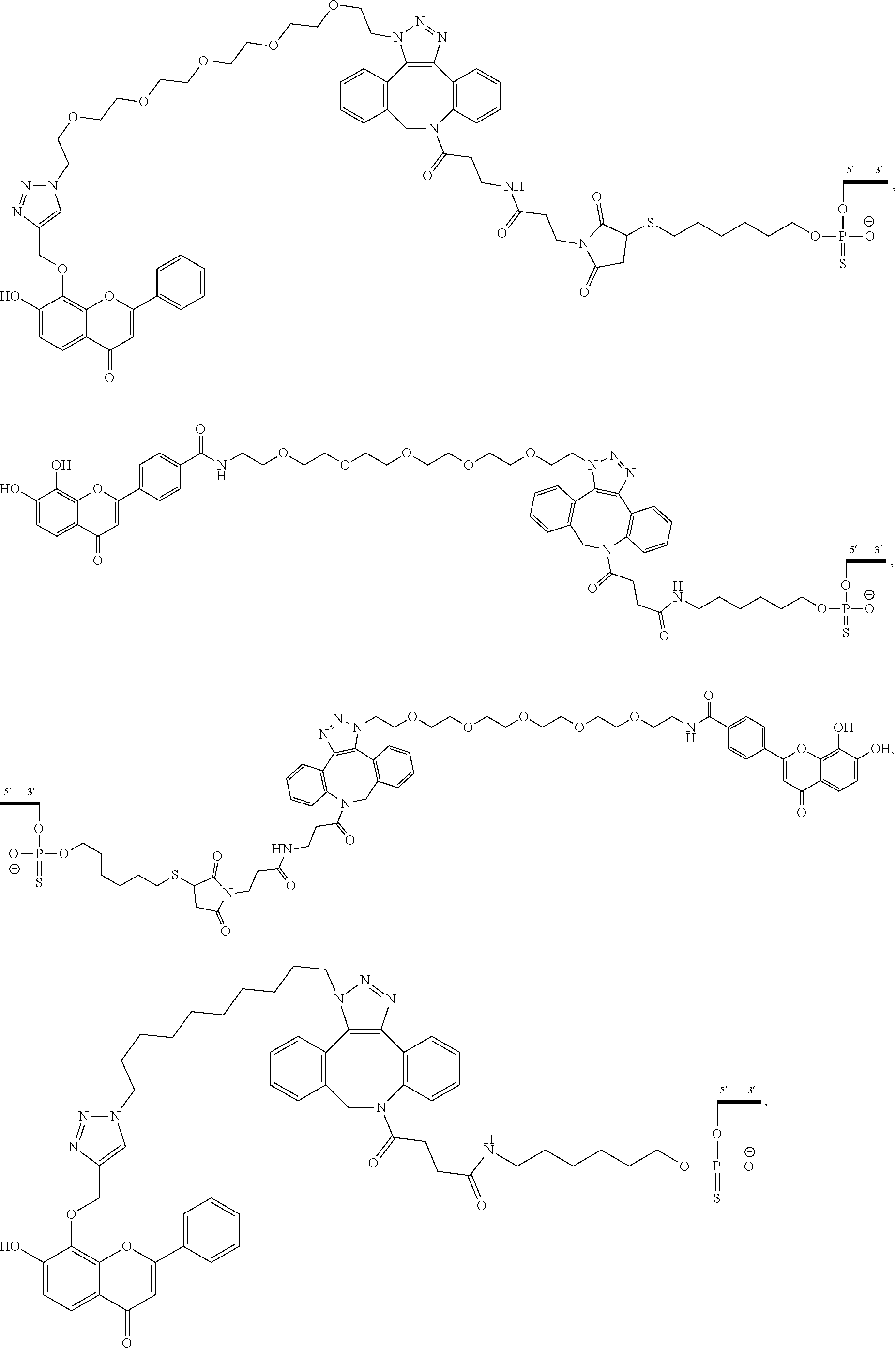

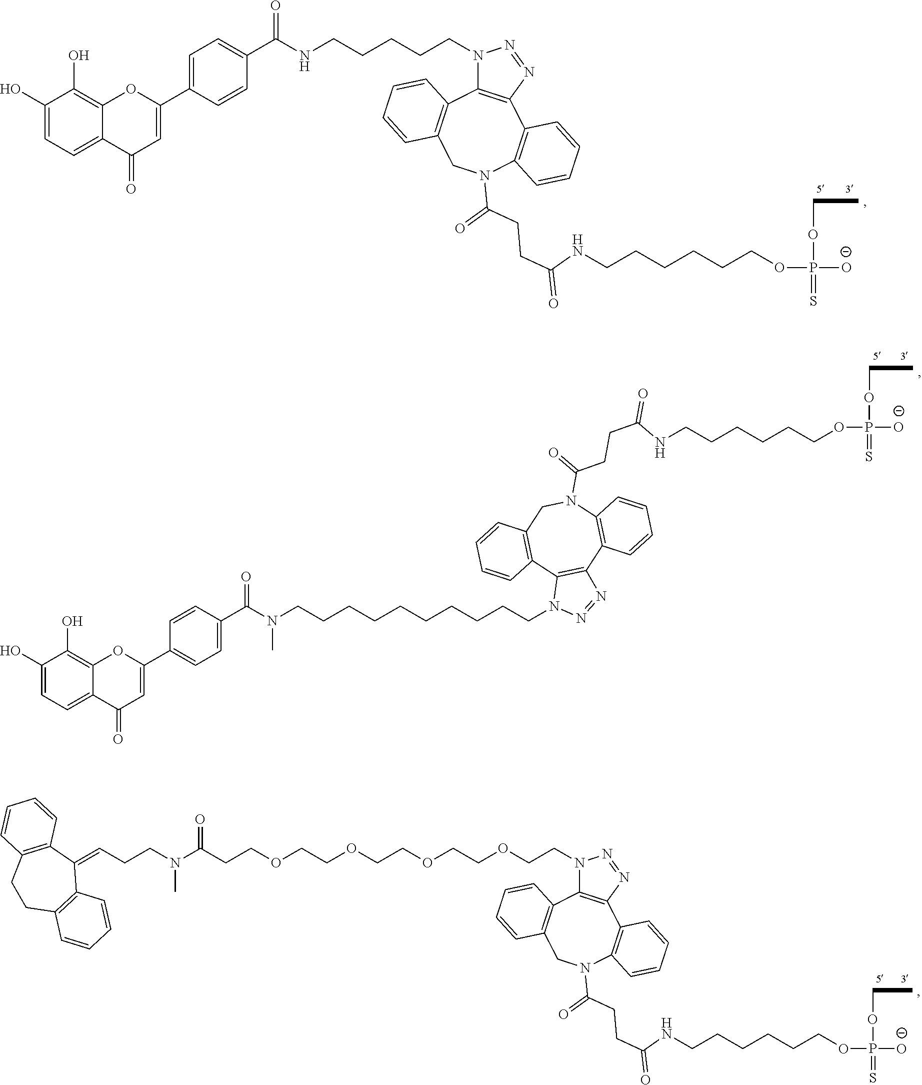

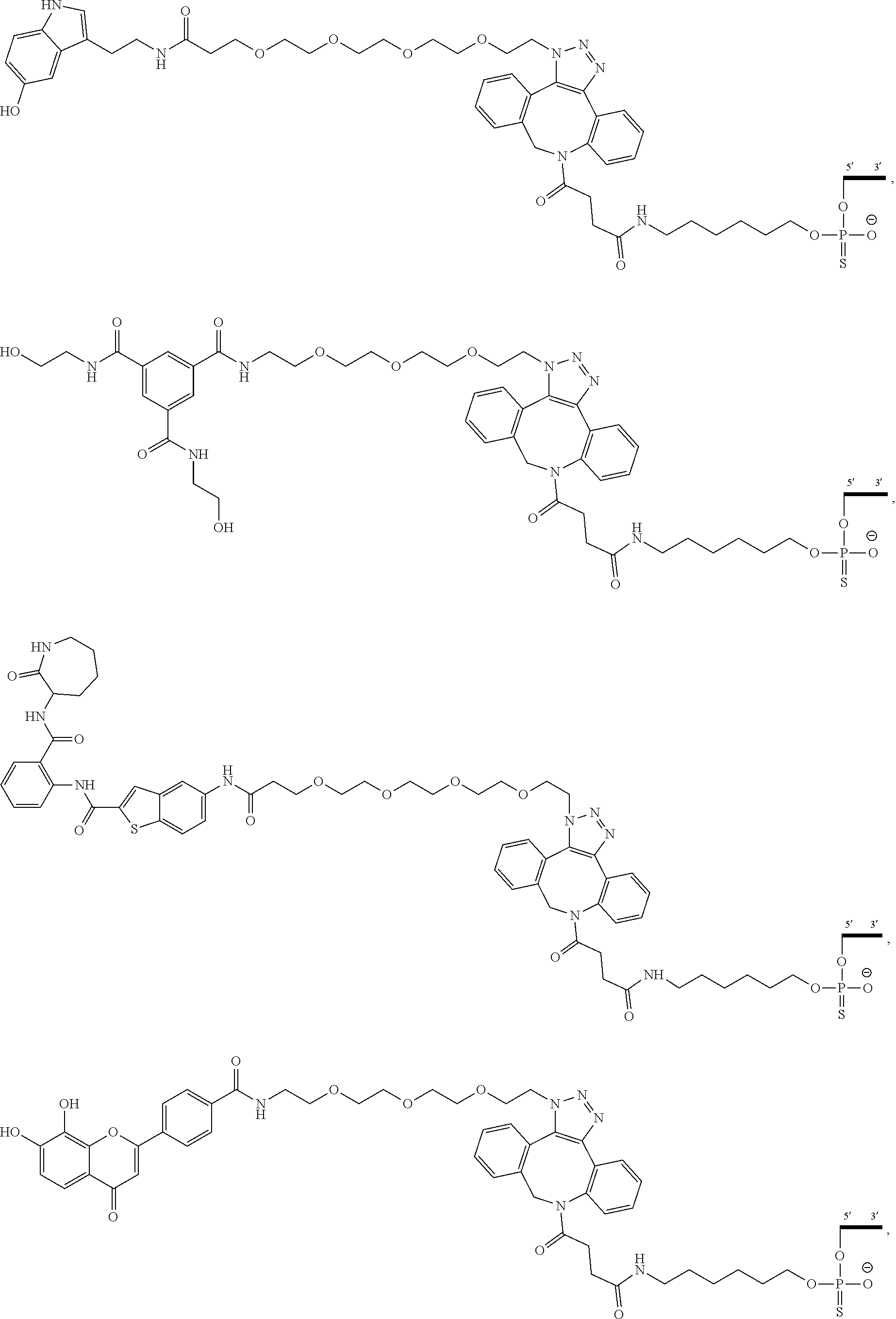

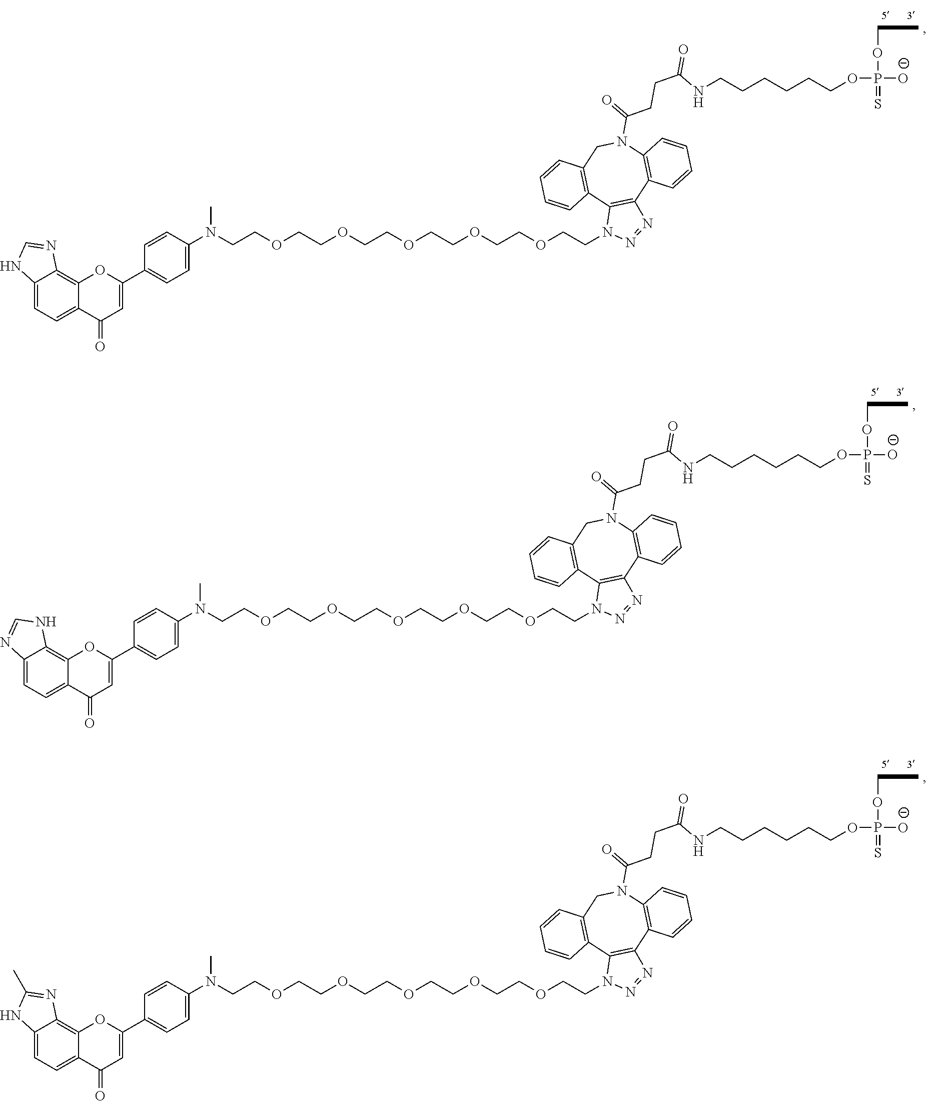

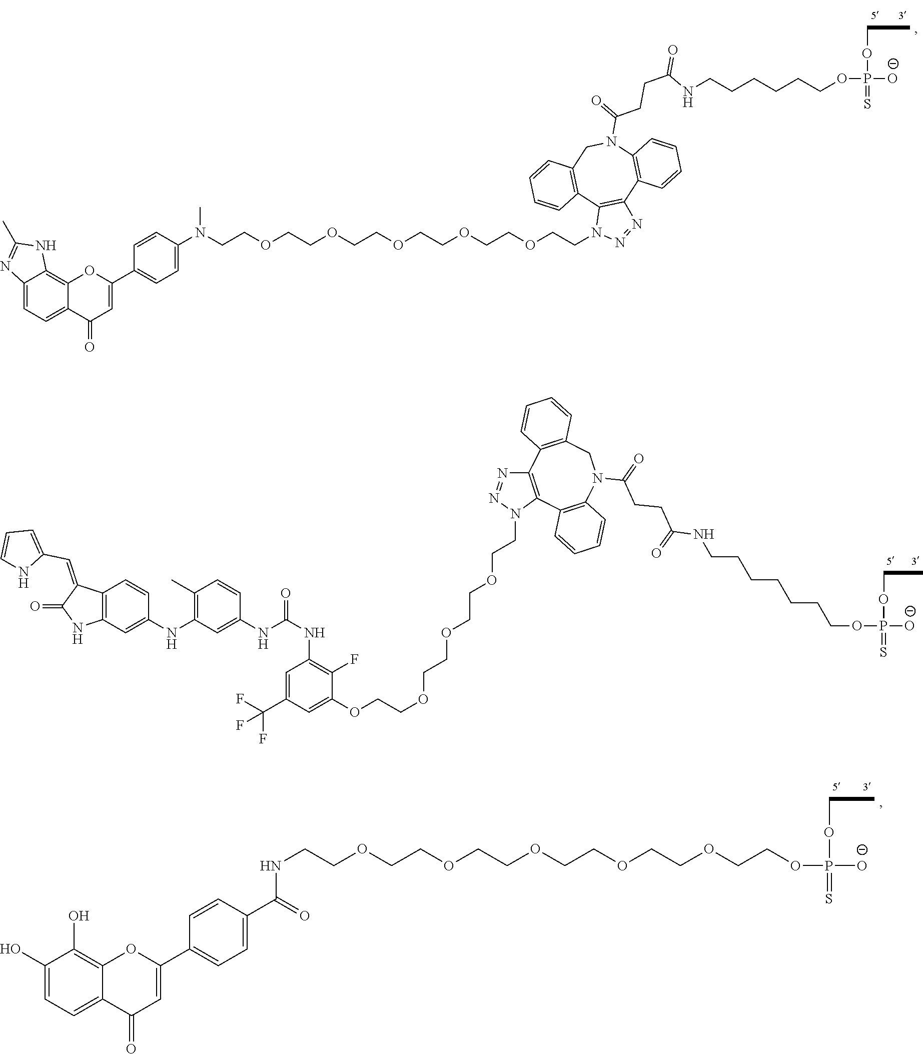

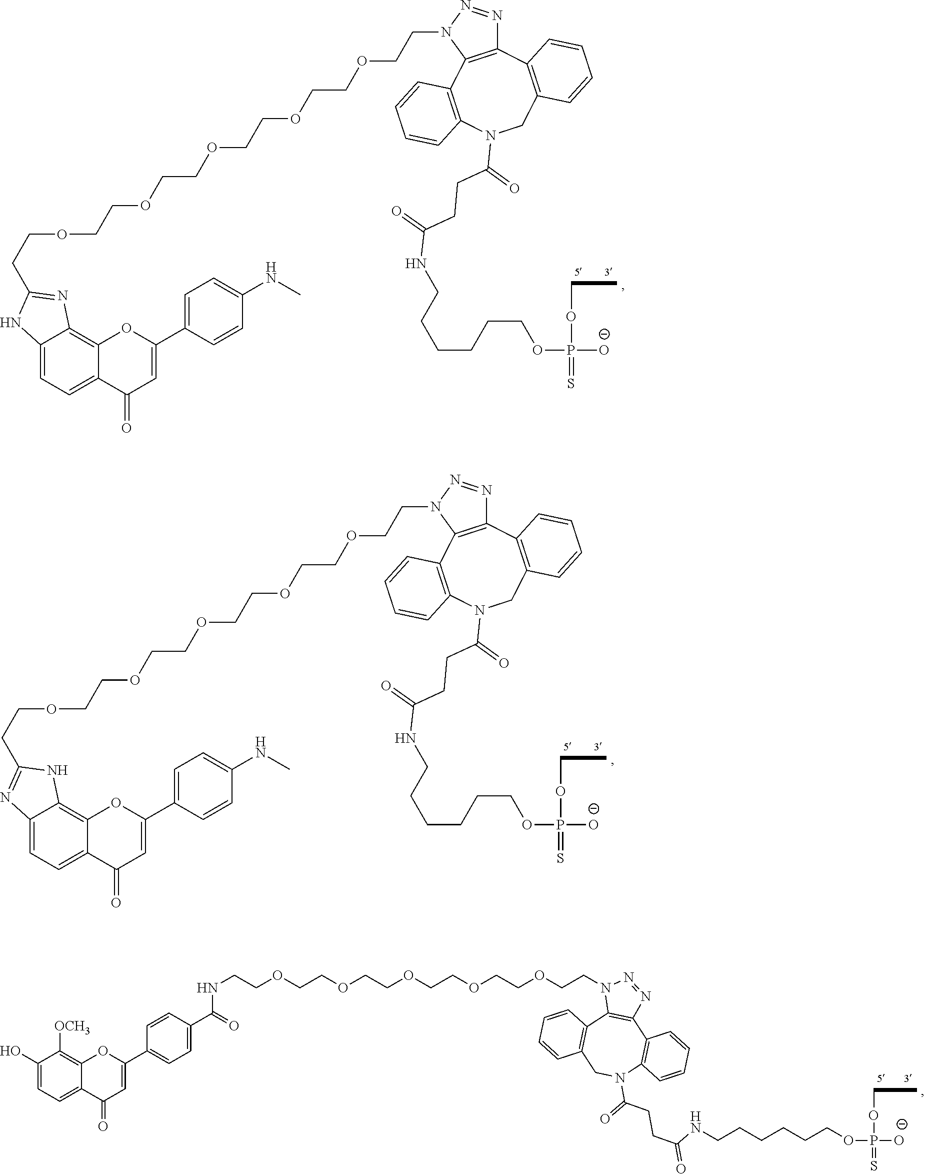

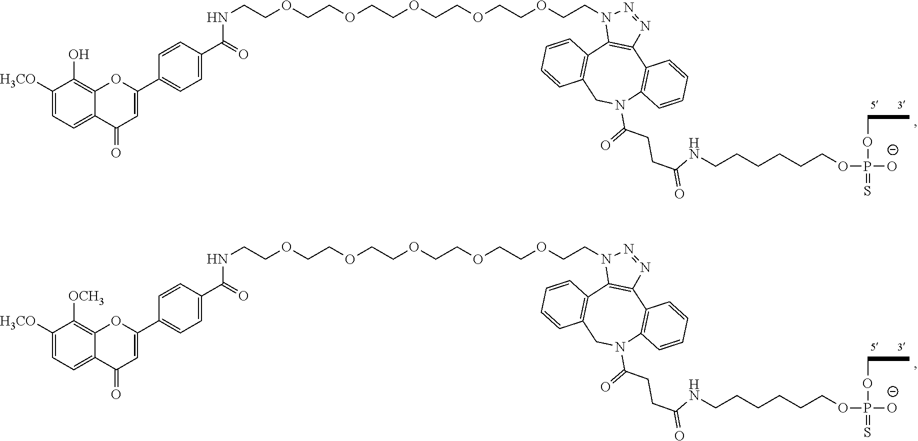

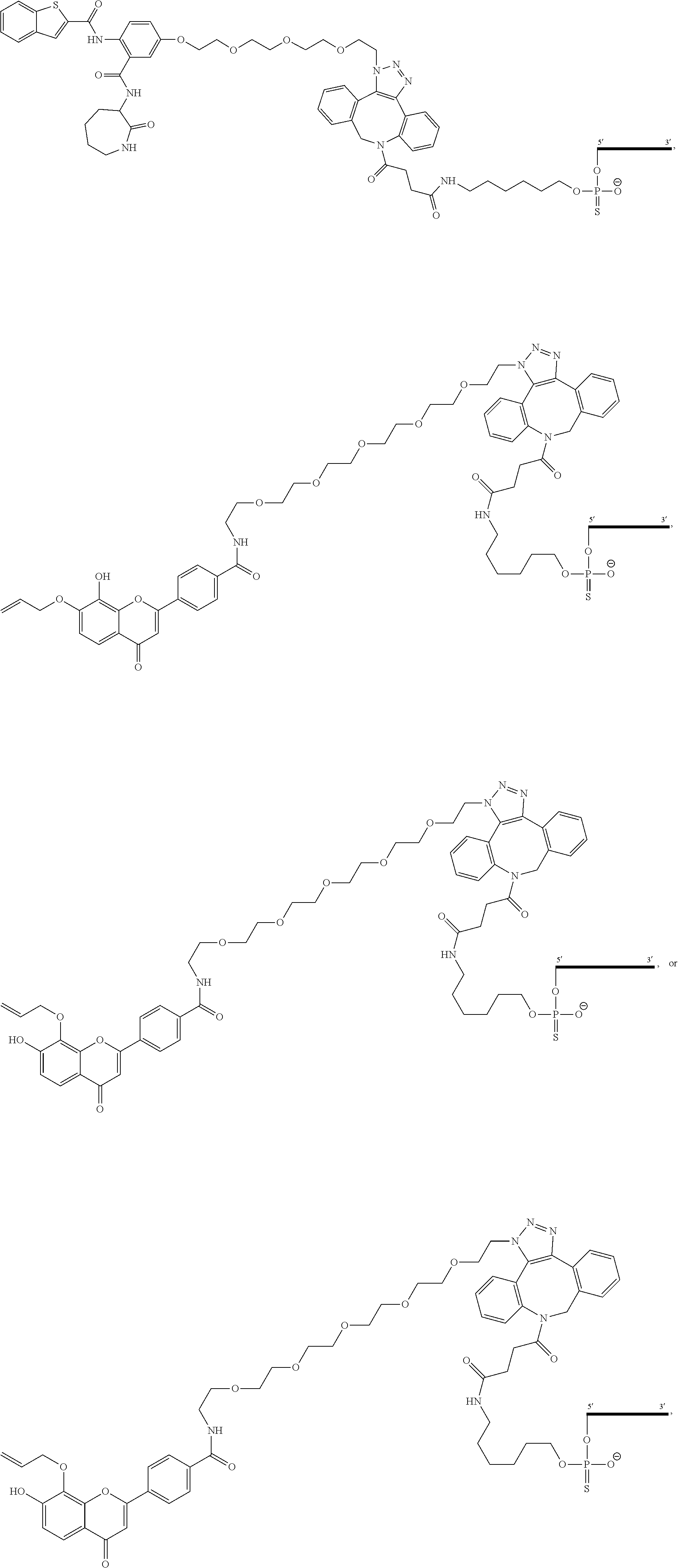

wherein [symbol] is an oligonucleotide.

In certain embodiments, there is provided a compound, or a stereoisomer, tautomer, prodrug, or salt thereof, comprising the structure:

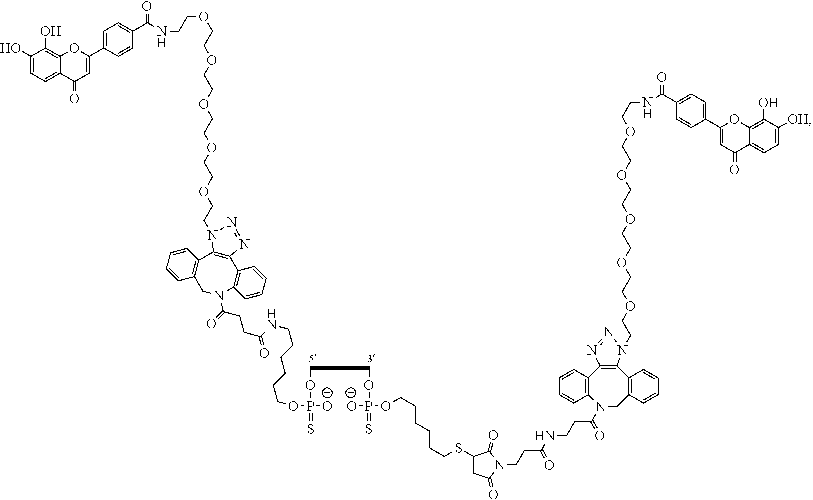

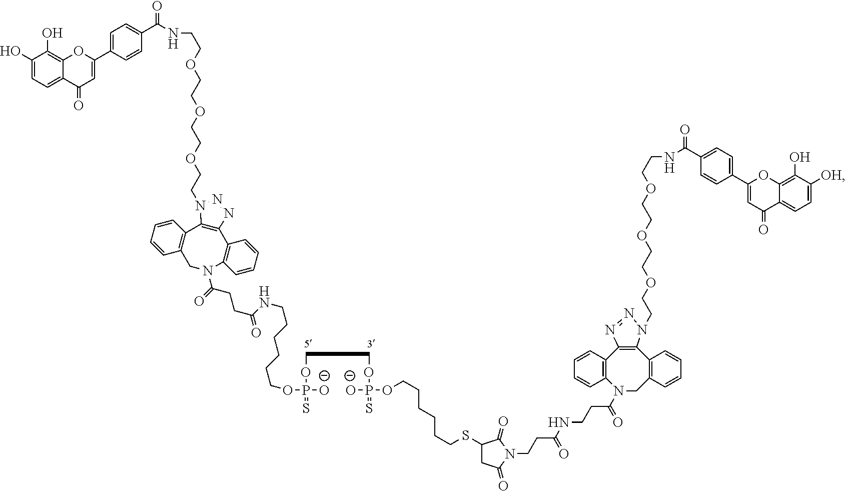

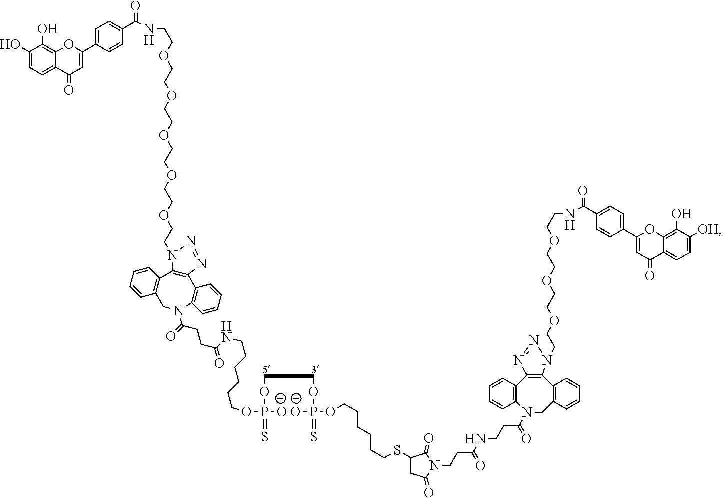

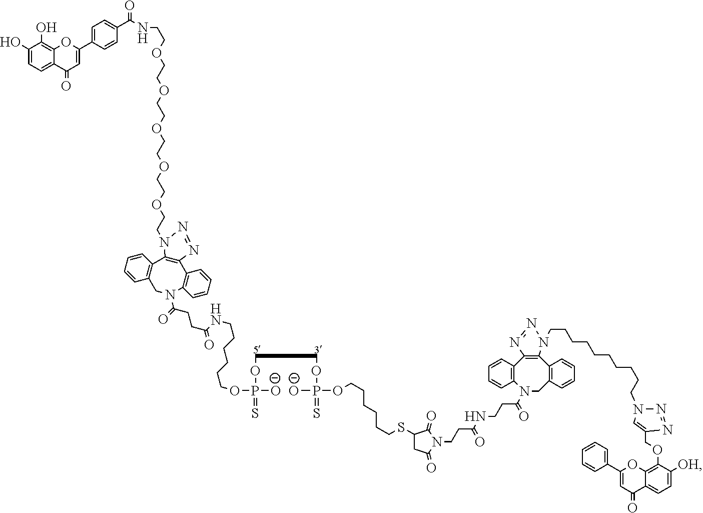

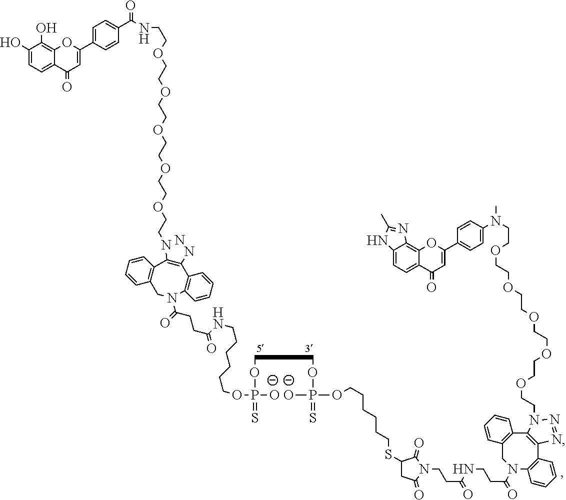

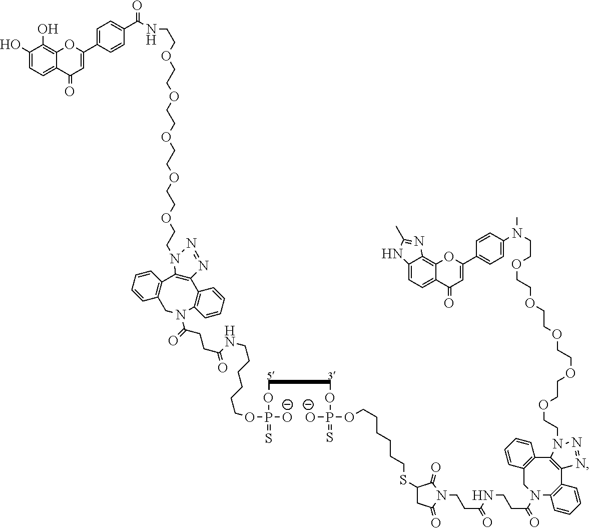

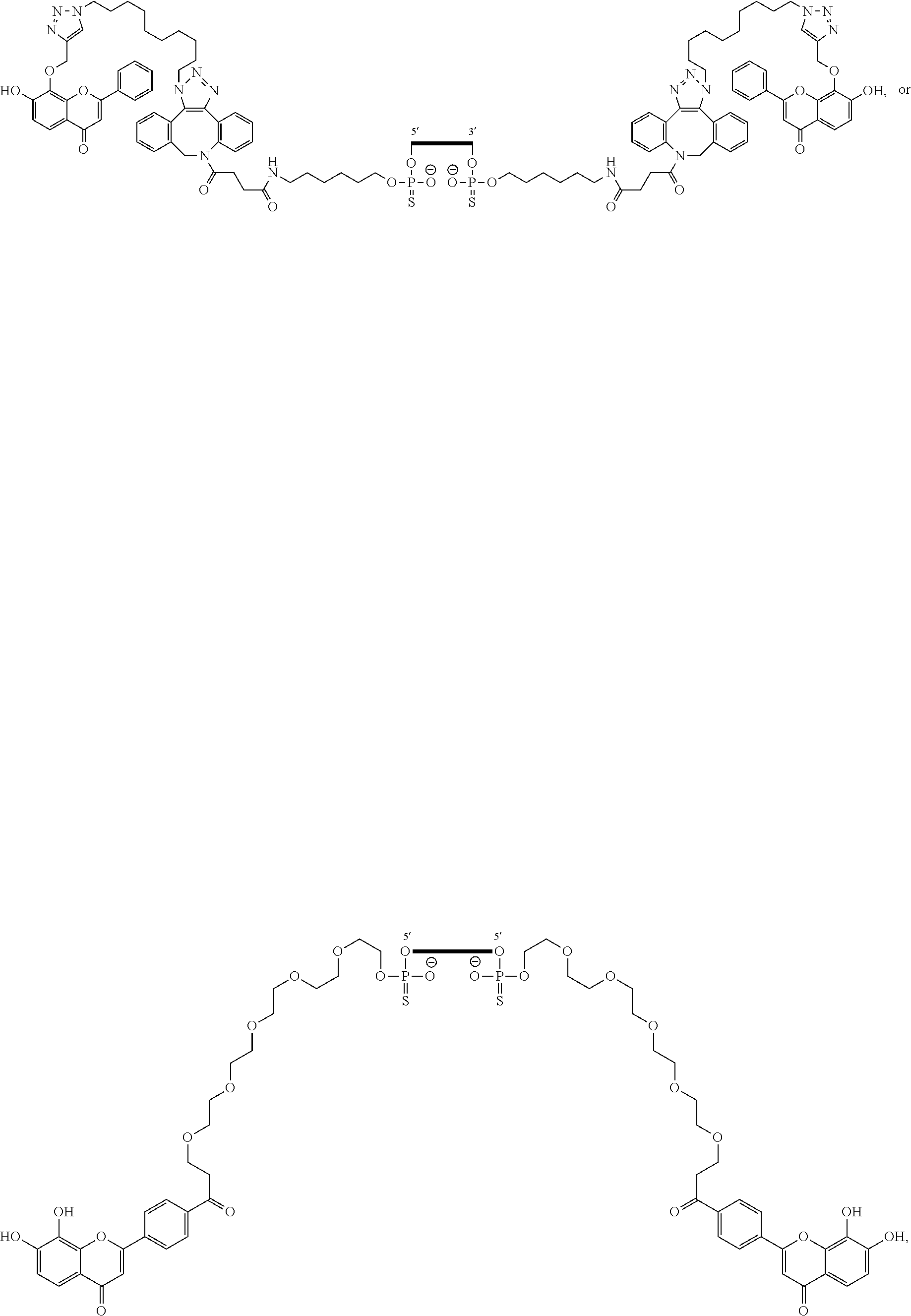

wherein [symbol] is an oligonucleotide.

In certain embodiments, the compounds disclosed herein are in salt form. In certain embodiments, salt is a potassium salt or a sodium salt.

In another aspect, there is provided a composition, comprising a compound disclosed herein and a pharmaceutically acceptable excipient.

In an aspect, there is provided a method for delivering a therapeutic oligonucleotide to the brain of a subject, comprising administration of a compound disclosed herein, or a stereoisomer, tautomer, prodrug, or salt thereof, to the subject. In certain embodiments, the therapeutic oligonucleotide is delivered to one or more brain regions selected from the group consisting of the striatum, the cerebellum, the brain stem, the hippocampus, the frontal cortex, and the spinal cord.

In an aspect, there is provided a method for treating or ameliorating a disease, disorder, or symptom thereof in a subject, comprising administration of a compound disclosed herein, or a stereoisomer, tautomer, prodrug, or salt thereof.

In an aspect, there is provided a method for treating or ameliorating a disease, disorder, or symptom thereof in a subject, comprising administration of a composition comprising a compound disclosed herein, or a stereoisomer, tautomer, prodrug, or salt thereof, and a pharmaceutically acceptable excipient. In certain embodiments, the disease, disorder, or symptom thereof is a central nervous system (CNS) disease, disorder, or symptom thereof. In certain embodiments, the disease, disorder, or symptom thereof is Alzheimer's disease, or a symptom thereof. In certain embodiments, the administration is intrathecal administration or intracerebroventricular (ICV) administration.

In an aspect, there is provided a method of delivering one or more cargo molecules to a cell or tissue of a subject in vivo, comprising administering to the subject a compound disclosed herein, or a stereoisomer, tautomer, prodrug, or salt thereof.

In an aspect, there is provided a method of delivering one or more cargo molecules to a cell or tissue of a subject in vivo, comprising administering to the subject a composition comprising a compound disclosed herein, or a stereoisomer, tautomer, prodrug, or salt thereof. In certain embodiments, the cell or tissue is CNS cell or tissue.

In an aspect, there is provided a method of making a compound, or a stereoisomer, tautomer, prodrug, or salt thereof, comprising contacting a precursor compound of any one of structural Formulae (A)-(F) disclosed herein with a compound of structural Formula (G) and/or (H):

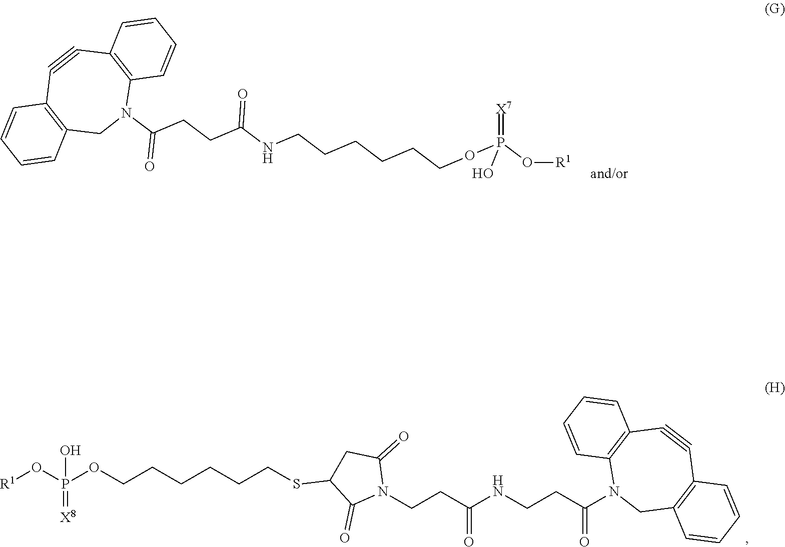

or a salt thereof,
wherein:
X⁷ and X⁸ are each independently O or S; and
R¹ is an oligonucleotide, a protecting group, a small molecule, a protein, an antibody, or a peptide.

In another aspect, the present disclosure provides methods for making any of the compounds provided herein, comprising one or more compounds and chemical transformations described herein, including Examples 2-29.

### DETAILED DESCRIPTION

### Definitions

It is to be understood that both the foregoing general description and the following detailed description are exemplary and explanatory only and are not restrictive of the embodiments, as claimed. Herein, the use of the singular includes the plural unless specifically stated otherwise. As used herein, the use of “or” means “and/or” unless stated otherwise. Furthermore, the use of the term “including” as well as other forms, such as “includes” and “included,” is not limiting. The section headings used herein are for organizational purposes only and are not to be construed as limiting the subject matter described.

Unless otherwise indicated, the following terms have the following meanings:

As used herein, the term “treating” a disorder encompasses ameliorating, mitigating and/or managing the disorder and/or conditions that may cause the disorder. The terms “treating” and “treatment” refer to a method of alleviating or abating a disease and/or its attendant symptoms. In accordance with the present disclosure, “treating” includes blocking, inhibiting, attenuating, protecting against, modulating, reversing the effects of, and reducing the occurrence of, e.g., the harmful effects of a disorder. As used herein, “inhibiting” encompasses preventing, reducing, and halting progression.

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high-performance liquid chromatography (HPLC). Particularly, in certain embodiments, the compound is at least 85% pure, more preferably at least 90% pure, more preferably at least 95% pure, and most preferably at least 99% pure.

The term “administration” or “administering” includes routes of introducing the compound(s) to a subject to perform their intended function. Examples of routes of administration which can be used include injection (subcutaneous, intravenous, parenterally, intraperitoneally, intrathecal), topical, oral, inhalation, rectal, and transdermal.

The term “effective amount” includes an amount effective, at dosages and for periods of time necessary, to achieve the desired result. An effective amount of compound may vary according to factors such as the disease state, age, and weight of the subject, and the ability of the compound to elicit a desired response in the subject. Dosage regimens may be adjusted to provide the optimum therapeutic response. An effective amount is also one in which any non-tolerable or detrimental effects (e.g., side effects) of the compound are outweighed by the therapeutically beneficial effects.

The phrases “systemic administration,” “administered systemically,” “peripheral administration” and “administered peripherally” as used herein mean the administration of a compound(s), oligonucleotide(s), drug, or other material, such that it enters the patient's circulatory system and, thus, is subject to metabolism and other like processes.

The term “therapeutically effective amount” refers to the amount of the compound being administered sufficient to prevent development of or alleviate to some extent one or more of the symptoms of the condition or disorder being treated.

A therapeutically effective amount of compound (i.e., an effective dosage) may range from about 0.005 μg/kg to about 200 mg/kg, preferably about 0.01 mg/kg to about 200 mg/kg, and more preferably about 0.015 mg/kg to about 30 mg/kg of body weight. In other embodiments, the therapeutically effect amount may range from about 1.0 pM to about 10 μM. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a compound can include a single treatment or, preferably, can include a series of treatments. In one example, a subject is treated with a compound in the range of between about 0.005 μg/kg to about 200 mg/kg of body weight, daily, weekly, monthly, quarterly, or yearly. In another example, a subject may be treated daily, weekly, monthly, quarterly, or yearly for several years in the setting of a chronic condition or illness. It will also be appreciated that the effective dosage of a compound used for treatment may increase or decrease over the course of a particular treatment.

The term “chiral” refers to molecules that have the property of non-superimposability of the mirror image partner, while the term “achiral” refers to molecules that are superimposable on their mirror image partner.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or(S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and(S)-, or (D)- and (L)-isomers may be prepared using chiral synthons or chiral reagents or resolved using conventional techniques. When the compounds described herein contain olefinic bonds or other centers of geometric asymmetry, and unless specified otherwise, it is intended that the compounds include both E and Z geometric isomers.

The term “tautomer,” as used herein, refers to one of two or more structural isomers which exist in equilibrium, and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure (i.e., the R and S configurations for each asymmetric center). Therefore, single stereochemical isomers as well as enantiomeric and diastereomeric mixtures of the present compounds are within the scope of the disclosure.

As used herein, “chirally enriched population” means a plurality of molecules of identical molecular formula, wherein the number or percentage of molecules within the population that contain a particular stereochemical configuration at a particular chiral center is greater than the number or percentage of molecules expected to contain the same particular stereochemical configuration at the same particular chiral center within the population if the particular chiral center were stereorandom. Chirally enriched populations of molecules having multiple chiral centers within each molecule may contain one or more stereorandom chiral centers. In certain embodiments, the molecules are modified oligonucleotides. In certain embodiments, the molecules are compounds comprising modified oligonucleotides.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

As used herein, “stereorandom chiral center” in the context of a population of molecules of identical molecular formula means a chiral center having a random stereochemical configuration. For example, in a population of molecules comprising a stereorandom chiral center, the number of molecules having the(S) configuration of the stereorandom chiral center may be but is not necessarily the same as the number of molecules having the (R) configuration of the stereorandom chiral center. The stereochemical configuration of a chiral center is considered random when it is the results of a synthetic method that is not designed to control the stereochemical configuration. In certain embodiments, a stereorandom chiral center is a stereorandom phosphorothioate internucleoside linkage.

The term “diastereomers” refers to stereoisomers with two or more centers of dissymmetry and whose molecules are not mirror images of one another.

The term “enantiomers” refers to two stereoisomers of a compound that are non-superimposable mirror images of one another. An equimolar mixture of two enantiomers is called a “racemic mixture” or a “racemate.”

The term “isomers” or “stereoisomers” refers to compounds that have identical chemical constitution but differ with regard to the arrangement of the atoms or groups in space.

The term “prodrug” is meant to indicate a compound that may be converted under physiological conditions or by solvolysis to a biologically active form of the compound (e.g., biologically active form of a nucleic acid) or analogue thereof as described herein. Thus, the term “prodrug” refers to a precursor of a biologically active compound (e.g., nucleic acid) or analogue thereof that is pharmaceutically acceptable. A prodrug may be inactive when administered to a subject, but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein. The term “prodrug” is also meant to include any covalently bonded carriers, which release the active compound in vivo when such prodrug is administered to a mammalian subject. Prodrugs of an active compound, as described herein, may be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a mammalian subject, cleaves to form a free hydroxy, free amino or free mercapto group, respectively. Examples of suitable prodrugs include, but are not limited to glutathione, acyloxy, thioacyloxy, 2-carboalkoxyethyl, disulfide, thiaminal, and enol ester derivatives of a phosphorus atom-modified nucleic acid. The term “pro-oligonucleotide” or “pronucleotide” or “nucleic acid prodrug” refers to an oligonucleotide which has been modified to be a prodrug of the oligonucleotide. Phosphonate and phosphate prodrugs can be found, for example, in Wiener et al., “Prodrugs or phosphonates and phosphates: crossing the membrane” Top. Curr. Chem.2015, 360:115-160, the entirety of which is herein incorporated by reference. Prodrugs that are converted to active forms through other mechanisms in vivo are also included. In aspects, the compounds of the present disclosure are prodrugs of any of the formulae herein.

The term “subject” refers to animals such as mammals, including, but not limited to, primates (e.g., humans), cows, sheep, goats, horses, dogs, cats, rabbits, rats, mice, and the like. In certain embodiments, the subject is a human.

The terms “a,” “an,” and “the” refer to “one or more” when used in this application, including the claims. Thus, for example, reference to “a sample” includes a plurality of samples, unless the context clearly is to the contrary (e.g., a plurality of samples), and so forth.

Throughout this specification and the claims, the words “comprise,” “comprises,” and “comprising” are used in a non-exclusive sense, except where the context requires otherwise.

As used herein, the term “about,” when referring to a value, is meant to encompass variations of, in some embodiments±20%, in some embodiments±10%, in some embodiments ±5%, in some embodiments±1%, in some embodiments±0.5%, and in some embodiments±0.1% from the specified amount, as such variations are appropriate to perform the disclosed methods or employ the disclosed compositions.

As used herein, the term “alkyl,” by itself or as part of another substituent, means, unless otherwise stated, a straight-chained (i.e., unbranched) or branched carbon chain (or carbon), or combination thereof, which may be fully saturated, mono-, (e.g., alkene or alkenyl) or polyunsaturated (e.g., alkyne or alkynyl) and can include mono-, di- and multivalent radicals, having the number of carbon atoms designated. For example, C₁-C₂₄ means 1 to 24 carbon atoms. A specified number of carbon atoms within this range includes, for example, C₁-C₂₀ alkyl (having 1-20 carbon atoms), C₁-C₁₂ alkyl (having 1-12 carbon atoms) and C₁-C₄ alkyl (having 1-4 carbon atoms).

The term “alkenyl” refers to an unsaturated hydrocarbon chain that may be a straight chain or branched chain, containing 2 to 12 carbon atoms and at least one carbon-carbon double bond. Alkenyl groups may be optionally substituted with one or more substituents.

The term “alkynyl” refers to an unsaturated hydrocarbon chain that may be a straight chain or branched chain, containing the 2 to 12 carbon atoms and at least one carbon-carbon triple bond. Alkynyl groups may be optionally substituted with one or more substituents.

The term “lower alkyl” refers to a C₁-C₆ alkyl chain. Examples of alkyl groups include methyl, ethyl, n-propyl, isopropyl, tert-butyl, and n-pentyl. Alkyl groups may be optionally substituted with one or more substituents.

The term “heteroalkyl” by itself or in combination with another term, means, unless otherwise stated, a stable straight or branched chain, or combinations thereof, including at least one carbon atom and at least one heteroatom (e.g., O, N, P, Si, and/or S), and wherein the nitrogen and sulfur atoms may optionally be oxidized, and the nitrogen heteroatom may optionally be quaternized. The heteroatom(s)(e.g., O, N, P, Si, and/or S) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, B, or P). A heteroalkyl moiety may include two optionally different heteroatoms (e.g., O, N, S, Si, B, and/or P). A heteroalkyl moiety may include three optionally different heteroatoms (e.g., O, N, S, Si, B, and/or P). A heteroalkyl moiety may include four optionally different heteroatoms (e.g., O, N, S, Si, B, and/or P). A heteroalkyl moiety may include five optionally different heteroatoms (e.g., O, N, S, Si, B, and/or P). A heteroalkyl moiety may include up to 8 or more optionally different heteroatoms (e.g., O, N, S, Si, B, and/or P).

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. “Cycloalkyl” is also meant to refer to bicyclic and polycyclic hydrocarbon rings such as, for example, bicyclo[2.2.1]heptane, bicyclo[2.2.2]octane, etc.

The term “haloalkyl” refers to an alkyl group that is substituted by one or more halo substituents. Examples of haloalkyl groups include fluoromethyl, difluoromethyl, trifluoromethyl, bromomethyl, chloromethyl, and 2,2,2-trifluoroethyl.

The term “arylalkenyl” refers to an unsaturated hydrocarbon chain that may be a straight chain or branched chain, containing 2 to 12 carbon atoms and at least one carbon-carbon double bond wherein one or more of the sp²-hybridized carbons of the alkenyl unit attaches to an aryl moiety. Alkenyl groups may be optionally substituted with one or more substituents.

The term “arylalkynyl” refers to an unsaturated hydrocarbon chain that may be a straight chain or branched chain, containing 2 to 12 carbon atoms and at least one carbon-carbon triple bond wherein one or more of the sp-hybridized carbons of the alkynyl unit attaches to an aryl moiety. Alkynyl groups may be optionally substituted with one or more substituents.

The sp²- or sp-hybridized carbons of an alkenyl group and an alkynyl group, respectively, may optionally be the point of attachment of the alkenyl or alkynyl groups.

The term “alkoxy” refers to an —O-alkyl substituent.

As used herein, the terms “halogen,” “hal,” or “halo” means —F, —Cl, —Br or —I.

The term “alkylthio” refers to an —S-alkyl substituent.

The term “alkoxyalkyl” refers to an -alkyl-O-alkyl substituent.

The term “haloalkoxy” refers to an —O-alkyl that is substituted by one or more halo substituents. Examples of haloalkoxy groups include trifluoromethoxy, and 2,2,2-trifluoroethoxy.

The term “haloalkoxyalkyl” refers to an -alkyl-O-alkyl′ where the alkyl′ is substituted by one or more halo substituents.

The term “haloalkylaminocarbonyl” refers to a —C(O)-amino-alkyl where the alkyl is substituted by one or more halo substituents.

The term “haloalkylthio” refers to an —S-alkyl that is substituted by one or more halo substituents. Examples of haloalkylthio groups include trifluoromethylthio, and 2,2,2-trifluoroethylthio.

The term “haloalkylcarbonyl” refers to an —C(O)-alkyl that is substituted by one or more halo substituents. An example of a haloalkylcarbonyl group includes trifluoroacetyl.

The term “cycloalkyl” refers to a hydrocarbon 3-8 membered monocyclic or 7-14 membered bicyclic ring system having at least one saturated ring or having at least one non-aromatic ring, wherein the non-aromatic ring may have some degree of unsaturation. Cycloalkyl groups may be optionally substituted with one or more substituents. In one embodiment, 0, 1, 2, 3, or 4 atoms of each ring of a cycloalkyl group may be substituted by a substituent. Representative examples of cycloalkyl group include cyclopropyl, cyclopentyl, cyclohexyl, cyclobutyl, cycloheptyl, cyclopentenyl, cyclopentadienyl, cyclohexenyl, cyclohexadienyl, and the like. The term “cycloalkoxy” refers to an —O-cycloalkyl substituent.

The term “cycloalkoxyalkyl” refers to an -alkyl-O-cycloalkyl substituent.

The term “cycloalkylalkoxy” refers to an —O-alkyl-cycloalkyl substituent.

The term “cycloalkylaminocarbonyl” refers to an —C(O)—NH-cycloalkyl substituent.

The term “aryl” refers to a hydrocarbon monocyclic, bicyclic, or tricyclic aromatic ring system. Aryl groups may be optionally substituted with one or more substituents. In one embodiment, 0, 1, 2, 3, 4, 5 or 6 atoms of each ring of an aryl group may be substituted by a substituent. Examples of aryl groups include phenyl, naphthyl, anthracenyl, fluorenyl, indenyl, azulenyl, and the like.

The term “aryloxy” refers to an —O-aryl substituent.

The term “arylalkoxy” refers to an —O-alkyl-aryl substituent.

The term “arylalkylthio” refers to an —S-alkyl-aryl substituent.

The term “arylthioalkyl” refers to an -alkyl-S-aryl substituent.

The term “arylalkylaminocarbonyl” refers to a —C(O)-amino-alkyl-aryl substituent.

The term “arylalkylsulfonyl” refers to an —S(O)₂-alkyl-aryl substituent.

The term “arylalkylsulfinyl” refers to an —S(O)-alkyl-aryl substituent.

The term “aryloxyalkyl” refers to an -alkyl-O-aryl substituent.

The term “alkylaryl” refers to an -aryl-alkyl substituent.

The term “arylalkyl” refers to an -alkyl-aryl substituent.

The term “heteroaryl” refers to an aromatic 5-8 membered monocyclic, 8-12 membered bicyclic, or 11-14 membered tricyclic ring system having 1-4 ring heteroatoms if monocyclic, 1-6 heteroatoms if bicyclic, or 1-9 heteroatoms if tricyclic, said heteroatoms selected from O, N, or S, and the remainder ring atoms being carbon (with appropriate hydrogen atoms unless otherwise indicated). Heteroaryl groups may be optionally substituted with one or more substituents. In one embodiment, 0, 1, 2, 3, or 4 atoms of each ring of a heteroaryl group may be substituted by a substituent. Heteroaryl groups may be fully unsaturated, or they may be partially unsaturated and partially saturated. Examples of heteroaryl groups include pyridyl, furanyl, thienyl, pyrrolyl, oxazolyl, oxadiazolyl, imidazolyl thiazolyl, isoxazolyl, quinolinyl, pyrazolyl, isothiazolyl, pyridazinyl, pyrimidinyl, pyrazinyl, triazinyl, isoquinolinyl, indazolyl, and the like.

The term “heteroarylalkyl” refers to an -alkyl-heteroaryl substituent.

The term “heteroaryloxy” refers to an —O-heteroaryl substituent.

The term “heteroarylalkoxy” refers to an —O-alkyl-heteroaryl substituent.

The term “heteroaryloxyalkyl” refers to an -alkyl-O-heteroaryl substituent.

The term “nitrogen-containing heteroaryl” refers to a heteroaryl group having 1-4 ring nitrogen heteroatoms if monocyclic, 1-6 ring nitrogen heteroatoms if bicyclic, or 1-9 ring nitrogen heteroatoms if tricyclic.

The term “heterocycloalkyl” refers to a nonaromatic 3-8 membered monocyclic, 7-12 membered bicyclic, or 10-14 membered tricyclic ring system comprising 1-3 heteroatoms if monocyclic, 1-6 heteroatoms if bicyclic, or 1-9 heteroatoms if tricyclic, said heteroatoms selected from O, N, S, B, P or Si, wherein the nonaromatic ring system is completely saturated. Heterocycloalkyl groups may be optionally substituted with one or more substituents. In one embodiment, 0, 1, 2, 3, or 4 atoms of each ring of a heterocycloalkyl group may be substituted by a substituent. Representative heterocycloalkyl groups include piperidinyl, piperazinyl, tetrahydropyranyl, morpholinyl, thiomorpholinyl, 1,3-dioxolane, tetrahydrofuranyl, tetrahydrothienyl, thiirenyl, and the like.

The term “heterocycloalkylalkyl” refers to an -alkyl-heterocycloalkyl substituent.

The term “alkylamino” refers to an amino substituent which is further substituted with one or two alkyl groups. The term “aminoalkyl” refers to an alkyl substituent which is further substituted with one or more amino groups. The term “hydroxyalkyl” or “hydroxylalkyl” refers to an alkyl substituent which is further substituted with one or more hydroxyl groups. The alkyl or aryl portion of alkylamino, aminoalkyl, mercaptoalkyl, hydroxyalkyl, mercaptoalkoxy, sulfonylalkyl, sulfonylaryl, alkylcarbonyl, and alkylcarbonylalkyl may be optionally substituted with one or more substituents.

The symbol “ [symbol] ” denotes the point of attachment of a chemical moiety to the remainder of a molecule or chemical formula.

The term “nucleobase” refers to nitrogen-containing biological compounds that form nucleosides. They include purine bases and pyrimidine bases. Five nucleobases—adenine (A), cytosine (C), guanine (G), thymine (T), and uracil (U)—are referred to as primary or canonical nucleobases. When a nucleobase is listed in a formula definition, it refers to that moiety covalently bonded to the recited formula.

The term “modified nucleobase” refers to derivatives of a nucleobase. Examples of modified nucleobases include, but are not limited to, xanthine, hypoxanthine, 7-methylguanine, 5,6-dihydrouracil, 5-methylcytosine, 5-hydroxymethylcytosine, purine, 2,6-diaminopurine, and 6,8-diaminopurine. When a modified nucleobase is listed in a formula definition, it refers to that moiety covalently bonded to the recited formula.

The term “substituent” and “substituent group” means an atom or group that replaces the atom or group of a named parent compound. For example, a substituent of a modified nucleoside is an atom or group that differs from the atom or group found in a naturally occurring nucleoside (e.g., a modified 2′-substituent is any atom or group at the 2′-position of a nucleoside other than H or OH). Substituent groups can be protected or unprotected. Substituents may also be further substituted with other substituent groups and may be attached directly or via a linking group such as an alkyl or hydrocarbyl group to the parent compound. Similarly, as used herein, “substituent” in reference to a chemical functional group means an atom or group of atoms that differs from the atom or group of atoms normally present in the named functional group. In certain embodiments, substituents on any group (such as, for example, alkyl, alkenyl, alkynyl, aryl, aralkyl, heteroaryl, heteroaralkyl, heteroalkyl, cycloalkyl, heterocycloalkyl) can be at any atom of that group, wherein any group that can be substituted (such as, for example, alkyl, alkenyl, alkynyl, aryl, aralkyl, heteroaryl, heteroaralkyl, heteroalkyl, cycloalkyl, heterocycloalkyl) can be optionally substituted with one or more substituents (which may be the same or different), each replacing a hydrogen atom. Examples of suitable substituents include, but are not limited to alkyl, alkenyl, alkynyl, cycloalkyl, heterocycloalkyl, aralkyl, heteroaralkyl, aryl, heteroaryl, halogen, haloalkyl, cyano, nitro, alkoxy, aryloxy, hydroxyl, hydroxylalkyl, oxo (i.e., carbonyl), carboxyl, formyl, alkylcarbonyl, alkylcarbonylalkyl, alkoxycarbonyl, alkylcarbonyloxy, aryloxycarbonyl, heteroaryloxy, heteroaryloxycarbonyl, thio, mercapto, mercaptoalkyl, arylsulfonyl, amino, aminoalkyl, dialkylamino, alkylcarbonylamino, alkylaminocarbonyl, alkoxycarbonylamino, alkylamino, arylamino, diarylamino, alkylcarbonyl, or arylamino-substituted aryl; arylalkylamino, aralkylaminocarbonyl, amido, alkylaminosulfonyl, arylaminosulfonyl, dialkylaminosulfonyl, alkylsulfonylamino, arylsulfonylamino, imino, carboxamido, carbamido, carbamyl, thioureido, thiocyanato, sulfoamido, sulfonylalkyl, sulfonylaryl, mercaptoalkoxy, N-hydroxyamidinyl, or N′-aryl, N″-hydroxyamidinyl. In certain embodiments, substituents on any group include alkyl, alkenyl, alkynyl, cycloalkyl, heterocycloalkyl, aralkyl, heteroaralkyl, aryl, heteroaryl, halogen, haloalkyl, cyano, nitro, alkoxy, aryloxy, hydroxyl, hydroxylalkyl, oxo (i.e., carbonyl), carboxyl, formyl, alkylcarbonyl, alkylcarbonylalkyl, alkoxycarbonyl, alkylcarbonyloxy, thiocarbonyl, thio, mercapto, mercaptoalkyl, arylsulfonyl, amino, aminoalkyl, dialkylamino, alkylcarbonylamino, alkylaminocarbonyl, alkoxycarbonylamino, alkylamino, arylamino, diarylamino, alkylcarbonyl, or arylamino-substituted aryl; arylalkylamino, aralkylaminocarbonyl, or amido. In certain embodiments, substituents on any group include alkyl, halogen, haloalkyl, cyano, nitro, alkoxy, hydroxyl, hydroxylalkyl, carboxyl, formyl, alkylcarbonyl, alkoxycarbonyl, alkylcarbonyloxy, thio, mercapto, mercaptoalkyl, amino, aminoalkyl, dialkylamino, alkylcarbonylamino, alkylaminocarbonyl, or alkylamino.

The term “protecting group” or “protecting moiety” refers to a substituent that is commonly employed to block or protect a particular functionality while reacting other functional groups on the compound, a derivative thereof, or a conjugate thereof, and includes a nitrogen protecting group when attached to a nitrogen atom, or an oxygen protecting group when attached to an oxygen atom. Nitrogen and oxygen protecting groups are well known in the art and include those described in detail in Protecting Groups in Organic Synthesis, T. W. Greene and P. G. M. Wuts, 3rd edition, John Wiley & Sons, 1999, incorporated herein by reference.

In certain embodiments, the substituent present on a nitrogen atom is a nitrogen protecting group (also referred to as an amino protecting group). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, carbocyclyl, heterocyclyl, aralkyl, aryl, and heteroaryl is independently substituted with 0, 1, 2, 3, 4, or 5 R^{dd} groups, and wherein each R^{aa}, R^{bb}, and R^{cc} is independently alkyl, cycloalkyl, aryl, or heteroaryl, each of which may be optionally substituted with 1-3 independent R^{dd}, and each R^{dd} is independently alkyl, alkenyl, alkynyl, cycloalkyl, heterocycloalkyl, aralkyl, heteroaralkyl, aryl, heteroaryl, halogen, haloalkyl, cyano, nitro, alkoxy, aryloxy, hydroxyl, hydroxylalkyl, oxo (i.e., carbonyl), carboxyl, formyl, alkylcarbonyl, alkylcarbonylalkyl, alkoxycarbonyl, alkylcarbonyloxy, aryloxycarbonyl, heteroaryloxy, heteroaryloxycarbonyl, thio, mercapto, mercaptoalkyl, arylsulfonyl, amino, aminoalkyl, dialkylamino, alkylcarbonylamino, alkylaminocarbonyl, alkoxycarbonylamino, alkylamino, arylamino, diarylamino, alkylcarbonyl, or arylamino-substituted aryl; arylalkylamino, aralkylaminocarbonyl, amido, alkylaminosulfonyl, arylaminosulfonyl, dialkylaminosulfonyl, alkylsulfonylamino, arylsulfonylamino, imino, carbamido, carbamyl, thioureido, thiocyanato, sulfoamido, sulfonylalkyl, sulfonylaryl, or mercaptoalkoxy. Nitrogen protecting groups are well known in the art and include those described in detail in Protecting Groups in Organic Synthesis, T. W. Greene and P. G. M. Wuts, 3rd edition, John Wiley & Sons, 1999, incorporated herein by reference.

Amide nitrogen protecting groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitrophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine, o-nitrobenzamide, and o-(benzoyloxymethyl)benzamide.

Carbamate nitrogen protecting groups (e.g., —C(═O)OR^{aa}) include, but are not limited to, methyl carbamate, ethyl carbamate, 9-fluorenylmethyl carbamate (Fmoc), 9-(2-sulfo) fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido) propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4 (trimethylammonium)benzyl carbamate, and 2,4,6-trimethylbenzyl carbamate.

Sulfonamide nitrogen protecting groups (e.g., —S(═O)₂R^{aa}) include, but are not limited to, p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl) mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to as a hydroxyl protecting group). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃, —P(═O)₂R^{aa}, —P(═O)(R^{aa})₂, —P(═O)(OR^{cc})₂, —P(═O)₂N(R^{bb})₂, and —P(═O)(NR^{bb})₂, wherein R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl) methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl) xanthenyl, 9-(9-phenyl-10-oxo) anthryl, 1,3-benzodisulfuran-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio) pentanoate (levulinoyldithioacetal), pivaloate, adamantoate, crotonate, 4-methoxycrotonate, benzoate, p-phenylbenzoate, 2,4,6-trimethylbenzoate (mesitoate), t-butyl carbonate (BOC), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl)ethyl carbonate (Psec), 2-(triphenylphosphonio)ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on a sulfur atom is a sulfur protecting group (also referred to as a thiol protecting group). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃, —P(═O)₂R^{aa}, —P(═O)(R^{aa})₂, —P(═O)(OR^{cc})₂, —P(═O)₂N(R^{bb})₂, and —P(═O)(NR^{bb})₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

The term “antisense oligonucleotide” or “antisense strand” means an oligonucleotide which includes a region that is complementary to a target nucleic acid.

The term “composition” or “pharmaceutical composition” means a mixture of substances suitable for administering to a subject. For example, a composition may comprise one or more compounds or salt thereof and a sterile aqueous solution.

The term “nucleic acid” refers to molecules composed of linked monomeric nucleotides or nucleosides. A nucleic acid includes, but is not limited to, ribonucleic acids (RNA), deoxyribonucleic acids (DNA), single-stranded nucleic acids, and double-stranded nucleic acids.

The term “nucleobase sequence” means the order of contiguous nucleobases in a nucleic acid or oligonucleotide independent of any sugar or internucleoside linkage.

The term “nucleoside” means a compound comprising a nucleobase and a sugar moiety. The nucleobase and sugar moiety are each, independently, unmodified or modified. “Modified nucleoside” means a nucleoside comprising a modified nucleobase and/or a modified sugar moiety. Modified nucleosides include abasic nucleosides, which lack a nucleobase.

The term “oligomeric compound” means a polymer of linked subunits. With reference to a protein, peptide, polypeptide, or antibody, “subunit” refers to an amino acid or peptide bond. With reference to an oligonucleotide, “subunit” refers to a nucleotide, nucleoside, nucleobase, or sugar, or a modified nucleotide, nucleoside, nucleobase, or sugar as provided herein.

The term “oligonucleotide” means a polymer of linked nucleosides (e.g., polynucleotide, nucleic acid, polymer of nucleotides), each of which can be modified or unmodified, independent from one another. Without limitation, an oligonucleotide may be comprised of ribonucleic acids (e.g., comprised of ribonucleosides), deoxyribonucleic acids (e.g., comprised of deoxyribonucleosides), modified nucleic acids (e.g., comprised of modified nucleobases, sugars, and/or phosphate groups), or a combination thereof. Examples of oligonucleotide compounds include single-stranded and double-stranded compounds, such as, oligonucleotides, antisense oligonucleotides, interfering RNA compounds (RNAi compounds), microRNA (miRNA) targeting oligonucleotides and miRNA mimics, occupancy-based compounds (e.g., mRNA processing or translation blocking compounds and splicing compounds). RNAi compounds include double-stranded compounds (e.g., short-interfering RNA (siRNA) and double-stranded RNA (dsRNA)) and single-stranded compounds (e.g., single-stranded siRNA (ssRNA), single-stranded RNAi (ssRNAi), short hairpin RNA (shRNA), and microRNA mimics) which work at least in part through the RNA-induced silencing complex (RISC) pathway resulting in sequence specific degradation and/or sequestration of a target nucleic acid through a process known as RNA interference (RNAi). The term “RNAi compound” is meant to be equivalent to other terms used to describe nucleic acid compounds that are capable of mediating sequence-specific RNA interference, for example, interfering RNA (iRNA), iRNA agent, RNAi agent, small interfering RNA, short interfering RNA, short interfering oligonucleotide, short interfering nucleic acid, short interfering modified oligonucleotide, chemically modified siRNA, and others. Additionally, the term “RNAi” is meant to be equivalent to other terms used to describe sequence-specific RNA interference.

The terms “target nucleic acid,” “target RNA,” and “nucleic acid target” all mean a nucleic acid capable of being targeted by compounds described herein.

The term “therapeutic compound” includes any pharmaceutical agent or compound that provides a therapeutic benefit to a subject. Therapeutic compounds include nucleic acids, oligomeric compounds, oligonucleotides, proteins, peptides, antibodies, small molecules, and other such agents.

“Target region” means a portion of a target nucleic acid to which one or more compounds is targeted.

“Targeting moiety” means a conjugate group that provides an enhanced affinity for a selected target, e.g., molecule, cell or cell type, compartment, e.g., a cellular or organ compartment, tissue, organ, or region of the body, as, e.g., compared to a compound absent such a moiety.

“Terminal group” means a chemical group or group of atoms that is covalently linked to a terminus of an oligonucleotide.

The terms “receptor tyrosine kinase” or “RTK” refer to a class of enzyme-linked transmembrane glycoproteins. One subfamily of RTKs is the tropomyosin-related kinase (Trk) receptor family, which is composed of three members: Tropomyosin Receptor Kinase A (TrkA), Tropomyosin Receptor Kinase B (TrkB), and Tropomyosin Receptor Kinase (TrkC).

The terms “Tropomyosin Receptor Kinase A” or “TrkA,” as may be used interchangeably herein, mean the receptor for Nerve Growth Factor (NGF) protein encoded by the NTRK1 gene.

The terms “Tropomyosin Receptor Kinase B” or “TrkB,” as may be used interchangeably herein, means the receptor for brain-derived neurotrophic factor (BDNF) protein encoded by the NTRK2 gene. TrkB is also known as tyrosine receptor kinase B, BDNF/NT-3 growth factors receptor and neurotrophic tyrosine kinase, receptor, type 2.

The terms “Tropomyosin Receptor Kinase C” or “TrkC,” as may be used interchangeably herein, mean the receptor for neurotropin-3 (NT-3) protein encoded by the NTF3 gene. In certain embodiments, the compounds disclosed herein are selective TrkB modulators. In other embodiments, the compounds disclosed herein are non-selective TrkB modulators (e.g., pan Trk ABC modulators). In certain embodiments, the compounds disclosed herein are TrkB agonists. In certain embodiments, the compounds disclosed herein are TrkB antagonists.

The term “sense oligonucleotide” or “sense strand” means the strand of a double-stranded compound that includes a region that is substantially complementary to a region of the antisense strand of the double-stranded compound.

The terms “microRNA” and “miRNA,” as may be used interchangeably herein, refer to short (e.g., about 20 to about 24 nucleotides in length)non-coding ribonucleic acids (RNAs) that are involved in post-transcriptional regulation of gene expression in multicellular organisms by affecting both the stability and translation of mRNAs. miRNAs are transcribed by RNA polymerase II as part of capped and polyadenylated primary transcripts (pri-miRNAs) that can be either protein-coding or non-coding. The primary transcript is cleaved by the Drosha ribonuclease III enzyme to produce a stem-loop precursor miRNA (pre-miRNA) approximately 70 nucleotides in length, which is further processed in the RNAi pathway. As part of this pathway, the pre-miRNA is cleaved by the cytoplasmic Dicer ribonuclease to generate the mature miRNA and antisense miRNA star (miRNA*) products. The mature miRNA is incorporated into an RNA-induced silencing complex (RISC), which recognizes target mRNAs through imperfect base pairing (i.e., partial complementarity) with the miRNA and most commonly results in translational inhibition or destabilization of the target mRNA. This mechanism is most often seen through the binding of the miRNA on the 3′ untranslated region (UTR) of the target mRNA, which can decrease gene expression by either inhibiting translation (for example, by blocking the access of ribosomes for translation) or directly causing degradation of the transcript. The term (i.e., miRNA) may be used herein to refer to any form of the subject miRNA (e.g., precursor, primary, and/or mature miRNA).

The terms “small interfering RNA,” “short interfering RNA,” and “siRNA,” as may be used interchangeably herein, refer to RNA molecules that present as non-coding double-stranded RNA (dsRNA) molecules of about 20 to about 24 nucleotides in length and are useful in RNA interference (RNAi). siRNA are often found with phosphorylated 5′ ends and hydroxylated 3′ ends, which 3′ ends typically have a 2-nucleotide overhang beyond the 5′ end of the anti-parallel strand (e.g., complementary strand of the dsRNA molecule). siRNA can interfere with the expression of specific genes through binding of target sequences (e.g., target nucleic acid sequences) to which they are complementary and promoting (e.g., facilitating, triggering, initiating) degradation of the mRNA, thereby preventing (e.g., inhibiting, silencing, interfering with) translation. After integration and separation into the RISC complex, siRNAs base-pair (e.g., full complementarity) to their target mRNA and cleave it, thereby preventing it from being used as a translation template. As discussed herein above, also part of the RNAi pathway, a miRNA-loaded RISC complex scans cytoplasmic mRNAs for potential complementarity (e.g., partial complementarity).

The term “ADAR recruiting molecule,” as may be used herein, refers to a nucleic acid that is configured to increase the concentration of Adenosine Deaminase Acting on Ribonucleic Acid (ADAR) enzyme in a locality around the nucleic acid. In some embodiments, an increased concentration is relative to the concentration in a given locality absent the ADAR recruiting molecule. In some embodiments, an ADAR recruiting molecule comprises a double-stranded RNA duplex.

The term “ADAR targeting molecule,” as may be used herein, refers to a nucleic acid that is configured to direct an ADAR molecule to a desirable location (e.g., locality). As used herein, the term “direct” refers to increasing the concentration of ADAR in the desirable location as compared to the concentration absent the ADAR targeting molecule. In some embodiments, the ADAR targeting molecule can be configured to control the desirable location by altering the sequence and/or properties of the nucleic acid (e.g., by modifications to the nucleobase, sugar, phosphate, or other component). In some embodiments, an ADAR targeting molecule comprises an ADAR recruiting molecule and a single-stranded guide nucleic acid. In some embodiments, an ADAR targeting molecule comprises a double-stranded RNA duplex and a single-stranded guide nucleic acid.

The term “single-stranded guide nucleic acid,” as may be used herein, refers to a nucleic acid of a single strand, which comprises a specific sequence that is at least partially complementary to a target sequence. In some embodiments, the target sequence is at, adjacent to, or in proximity to, a locality where it is desirable to modulate ADAR concentration. In some embodiments, the level of complementarity is sufficient to facilitate binding (e.g., annealing) of the single-stranded guide nucleic acid to the target sequence.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

The term “isotopic variant” refers to a therapeutic agent (e.g., a compound and/or modified oligonucleotide disclosed herein) that contains an unnatural proportion of an isotope at one or more of the atoms that constitute such a therapeutic agent. In certain embodiments, an “isotopic variant” of a therapeutic agent contains unnatural proportions of one or more isotopes, including, but not limited to, hydrogen (H), deuterium (²H), tritium (³H), carbon-11 (¹¹C), carbon-12 (¹²C), carbon-13 (¹³C), carbon-14 (¹⁴C), nitrogen-13 (¹³N), nitrogen-14 (¹⁴N), nitrogen-15 (¹⁵N), oxygen-14 (¹⁴O), oxygen-15 (¹⁵O), oxygen-16 (¹⁶O), oxygen-17 (¹⁷O), oxygen-18 (¹⁸O), fluorine-17 (¹⁷F), fluorine-18 (¹⁸F), phosphorus-31 (³¹P), phosphorus-32 (³²P), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵Cl), chlorine-36 (³⁶Cl), chlorine-37 (³⁷Cl), bromine-79 (⁷⁹Br), bromine-81 (⁸¹Br), iodine 123 (¹²³I), iodine-125 (¹²⁵I), iodine-127 (¹²⁷I), iodine-129 (¹²⁹I), and iodine-131 (¹³¹I). In certain embodiments, an “isotopic variant” of a therapeutic agent contains unnatural proportions of one or more isotopes, including, but not limited to, hydrogen (H), deuterium (²H), tritium (³H), carbon-11 (¹¹C), carbon-12 (¹²C), carbon-13 (¹³C), carbon-14 (¹⁴C), nitrogen-13 (¹³N), nitrogen-14 (¹⁴N), nitrogen-15 (¹⁵N), oxygen-14 (¹⁴O), oxygen-15 (¹⁵O), oxygen-16 (¹⁶O), oxygen-17 (¹⁷O), oxygen-18 (¹⁸O), fluorine-17 (¹⁷F), fluorine-18 (¹⁸F), phosphorus-31 (³¹P), phosphorus-32 (³²P), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵Cl), chlorine-36 (³⁶Cl), chlorine-37 (³⁷Cl), bromine-79 (⁷⁹Br), bromine-81 (⁸¹Br), iodine 123 (¹²³I), iodine-125 (¹²⁵I), iodine-127 (¹²⁷I), iodine-129 (¹²⁹I), and iodine-131 (¹³¹I).

It will be understood that, in a therapeutic agent (e.g., a compound and/or modified oligonucleotide disclosed herein), any hydrogen can be ²H, for example, or any carbon can be ¹³C, for example, or any nitrogen can be ¹⁵N, for example, or any oxygen can be ¹⁸O, for example, where feasible according to the judgment of one of skill. In certain embodiments, an “isotopic variant” of a therapeutic agent contains unnatural proportions of deuterium (D).

“Modified oligonucleotide” means an oligonucleotide, wherein at least one sugar, nucleobase, or internucleoside linkage is modified.

“Nucleobase sequence” means the order of contiguous nucleobases in a nucleic acid or oligonucleotide independent of any sugar or internucleoside linkage.

The term “oligomeric duplex” means a duplex formed by two oligomeric compounds having complementary nucleobase sequences. Each oligomeric compound of an oligomeric duplex may be referred to as a “duplexed oligomeric compound.” The oligonucleotides of each oligomeric compound of an oligomeric duplex may include non-complementary overhanging nucleosides. In some embodiments, the terms “duplexed oligomeric compound” and “modified oligonucleotide” are used interchangeably. In other embodiments, the terms “oligomeric duplex” and “compound” are used interchangeably.

“Phosphorothioate linkage” means a modified phosphate linkage in which one of the non-bridging oxygen atoms is replaced with a sulfur atom. Modified phosphate linkages where at least one of the oxygen linked to the phosphate has been replaced or the phosphate group has been replaced by a non-phosphorous group, are also referred to as “non-phosphodiester internucleoside linkage” or “non-phosphodiester linker.

The terms “RNA interference compound,” “RNAi compound,” and/or “iRNA agent” mean a compound that acts, at least in part, through an RNA-induced silencing complex (RISC) pathway or Ago2, but not through RNase H, to modulate a target nucleic acid and/or protein encoded by a target nucleic acid. RNAi compounds include, but are not limited to double-stranded siRNA, single-stranded siRNA, and microRNA, including microRNA mimics.

### Certain Embodiments

In certain embodiment, a compound comprises a TrkB ligand and one or more linker moieties. In certain embodiments, the compound is selected from any of formulae I, I′, II, II′, II″, II′″, II-a, II-b, II-c, III, III-a, III-b, III-c, III-c-1, IV, IV-a, IV-b, V, VI, VI′, VII, VII-a, VII-b, VII-c, VII-c-1, VII-c-2, VII-d, VII-d-1, VII-d-2, VIII-a, VIII-a-1, VIII, IX IX-a, IX-a-1, IX-a-2, X, X-a, XI, and XI-a, or a salt thereof as described herein. In certain embodiments, the one or more linker moieties (L₁, L₂, L₃, and L₄) links the TrkB ligand to a therapeutic, prophylactic, or diagnostic agent. In certain embodiments, the compound further comprises a therapeutic, prophylactic, or diagnostic agent. In certain embodiments, the therapeutic, prophylactic, or diagnostic agent is a small molecule, or an oligomeric compound. In certain embodiments, the oligomeric compound comprises a protein, peptide, antibody, oligonucleotide, or combination thereof.

In certain embodiments, an oligomeric compound is any of those described herein. In certain embodiments, the oligomeric compound is about 10-50 subunits in length. In certain embodiments the oligomeric compound is an oligonucleotide. In certain embodiments, an oligonucleotide is any of those described herein. In certain embodiments, the oligonucleotide is 8 to 80 linked nucleosides in length, 12-30 linked nucleosides in length, 12-30 linked nucleosides in length, or 15-30 linked nucleosides in length.

In certain embodiments, the oligonucleotide is a modified oligonucleotide comprising at least one modified internucleoside linkage, at least one modified sugar, or at least one modified nucleobase.

In certain embodiments, the oligonucleotide is single-stranded. In certain embodiments, the oligonucleotide is double-stranded. In certain embodiments, the oligonucleotide comprises ribonucleic acids (e.g., comprised of ribonucleosides), deoxyribonucleic acids (e.g., comprised of deoxyribonucleosides), or a combination thereof. In certain embodiments, the oligonucleotide is a small interfering RNA (siRNA), a microRNA (miRNA) antagonist, an miRNA mimic, an ADAR recruiting molecule, an ADAR targeting molecule, a guide RNA, an antisense oligonucleotide, a short hairpin RNA (shRNA), or combinations thereof.

In certain embodiments, a linker is an optionally substituted PEG linker. In certain embodiments, the PEG linker is five PEG units in length. In certain embodiments, the PEG linker is four PEG units in length. In certain embodiments, the PEG linker is three PEG units in length. In certain embodiments, a linker is an optionally substituted alkyl linker. In certain embodiments, a linker is an optionally substituted C₁₋₂₀ alkyl linker. In certain embodiments, the linker is an optionally substituted C₈ alkyl linker. In certain embodiments, a linker is an optionally substituted heteroaryl linker. In certain embodiments, a linker is an optionally substituted partially unsaturated heteroaryl linker. In certain embodiments, a linker comprises the structure

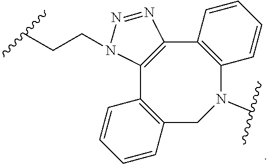 [SMILES: *CCn1nnc2c1-c1ccccc1CN(*)c1ccccc1-2]

In certain embodiments, a linker is an optionally substituted heteroalkyl linker. In certain embodiments, the heteroalkyl linker is substituted with one or more ═O substituents. In some embodiments, the heteroalkyl linker comprises two substituents joined together to form an optionally substituted carbocyclyl ring. In certain embodiments, a linker comprises the structure

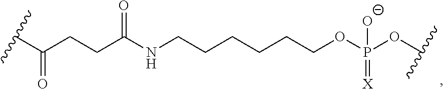

wherein X is O or S. In certain embodiments, a linker comprises the structure

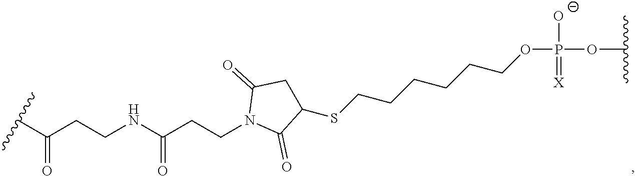

wherein X is O or S.

In certain embodiments, a linker comprises the structure

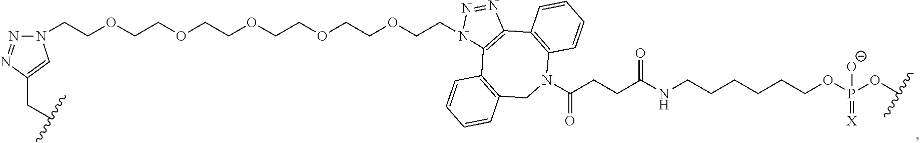

wherein X is O or S. In certain embodiments, a linker comprises the structure

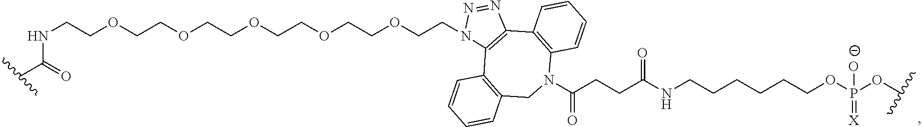

wherein X is O or S. In certain embodiments, a linker comprises the structure

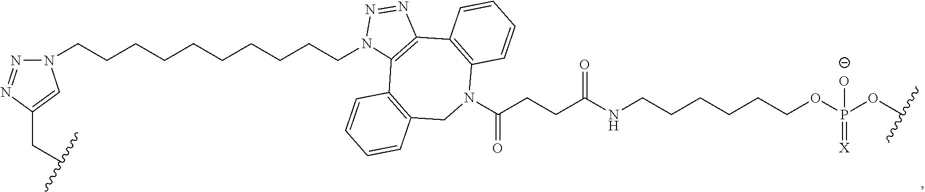

wherein X is O or S. In certain embodiments, a linker comprises the structure

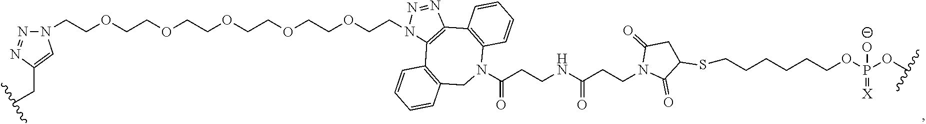

wherein X is O or S. In certain embodiments, a linker comprises the structure

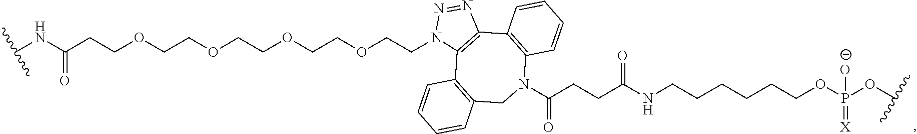

wherein X is O or S. In certain embodiments, a linker comprises the structure

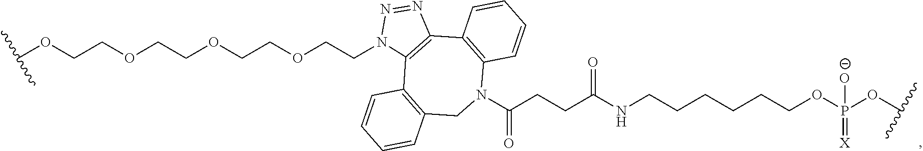

wherein X is O or S.

In certain embodiments, a compound comprises or consists of one of the structures:

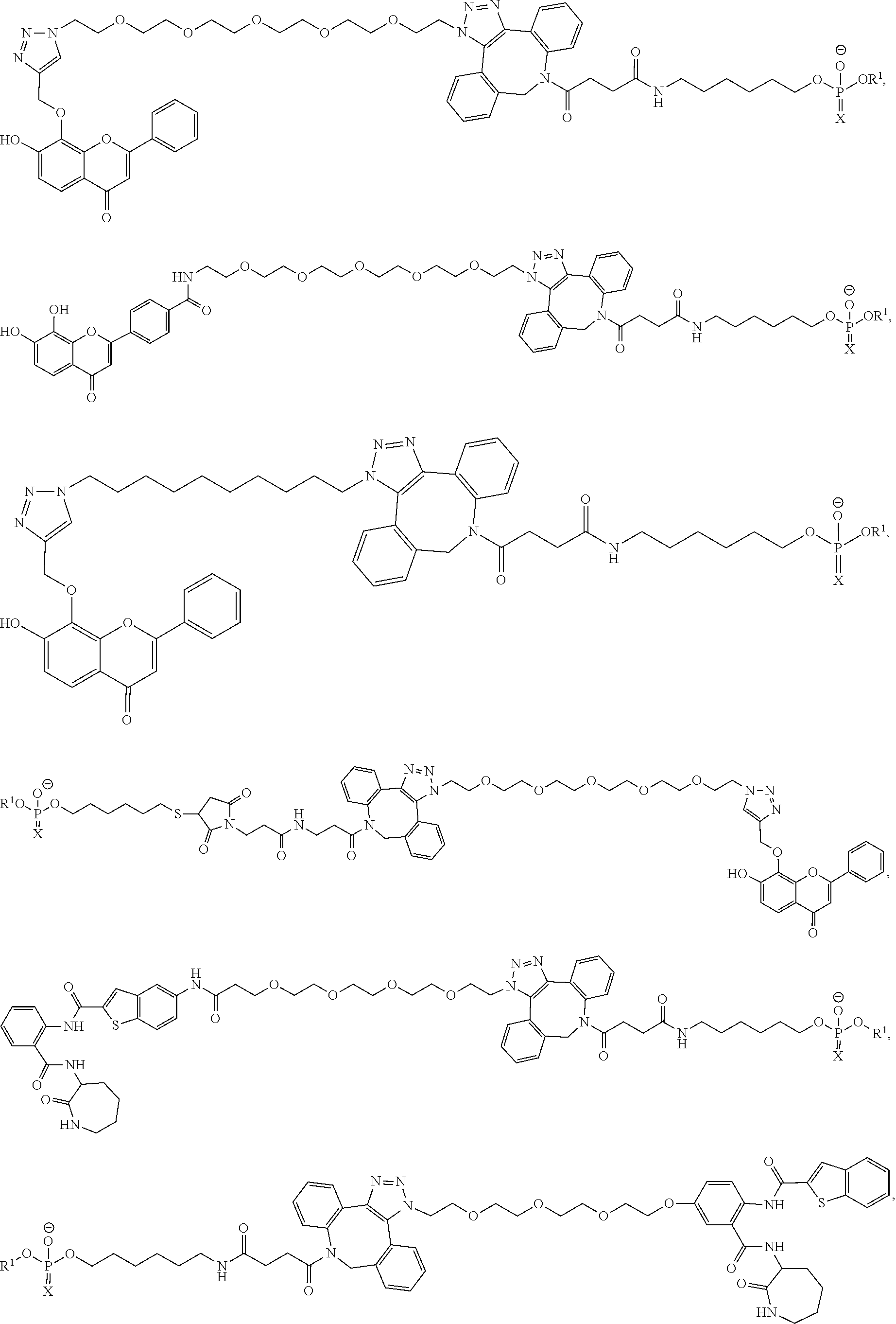

or a salt thereof, wherein X is O or S.

In some embodiments, X is O. In some embodiments, X is S.

In some embodiments, R¹ comprises an oligonucleotide. In some embodiments, the oligonucleotide is attached at its 5′ end. In some embodiments, the oligonucleotide is attached at its 3′ end. In some embodiments, the oligonucleotide is attached at an internal position on the oligonucleotide. In some embodiments the internal position is at an internucleoside linkage. In some embodiments, R¹ comprises an oligonucleotide conjugated to one or more additional TrkB ligands. In some embodiments, the oligonucleotide is conjugated to two, three, four, five, or more than five additional TrkB ligands. In certain embodiments, the additional TrkB ligands are conjugated to the oligonucleotide at the 5′ end of the oligonucleotide, the 3′ end of the oligonucleotide, one or more internal positions on the oligonucleotide, or any combination thereof. In certain embodiments, the oligonucleotide is a modified oligonucleotide.

Certain embodiments provide a composition comprising a compound of any embodiment herein, and a pharmaceutically acceptable carrier or excipient.

Certain embodiments provide a composition comprising a compound of any embodiment herein, for use in therapy.

In certain embodiments, a method for delivering an agent to cell comprises contacting the cell with the compound of any embodiments herein, thereby delivering the agent to the cell. In certain embodiments, the cell expresses TrkB on the surface of the cell. In certain embodiments, the cell is a brain cell. In certain embodiments the cell is a cell of the frontal cortex. In certain embodiments, the cell is a cell of the striatum. In certain embodiments, the cell is a cell of the cerebellum. In certain embodiments, the cell is a cell of the brain stem. In certain embodiments, the cell is a cell of the hippocampus. In certain embodiments, the cell is a cell of the spinal cord. In certain embodiments, the agent is a therapeutic agent or diagnostic agent. In certain embodiments the cell is in an animal.

In certain embodiments, a method of modulating the expression of a nucleic acid target in a cell comprises contacting the cell with the compound of any embodiments herein, thereby modulating expression of the nucleic acid target in the cell. In certain embodiments, the cell expresses TrkB on the surface of the cell. In certain embodiments, the cell is a brain cell. In certain embodiments the cell is a cell of the frontal cortex. In certain embodiments, the cell is a cell of the striatum. In certain embodiments, the cell is a cell of the cerebellum. In certain embodiments, the cell is a cell of the brain stem. In certain embodiments, the cell is a cell of the hippocampus. In certain embodiments, the cell is a cell of the spinal cord. In certain embodiments, the agent is a therapeutic agent or diagnostic agent. In certain embodiments, contacting the cell with the compound the compound of any embodiments herein inhibits expression of the nucleic acid target. In certain embodiments, the nucleic acid target is pre-mRNA, mRNA, non-coding RNA, or miRNA. In certain embodiments the cell is in an animal.

In certain embodiments, a method of modulating the expression of a nucleic acid target in a subject comprises administering to the subject any of the compounds or compositions provided herein, thereby modulating expression of the nucleic acid target in the subject. In certain embodiments, the expression of the nucleic acid is modulated in a cell of the subject that expresses TrkB on the surface of the cell. In certain embodiments, the expression of the nucleic acid is modulated in a brain cell. In certain embodiments, the cell expressing TrkB on its surface is a brain cell. In certain embodiments the brain cell is a cell of the frontal cortex. In certain embodiments, the brain cell is a cell of the striatum. In certain embodiments, the brain cell is a cell of the cerebellum. In certain embodiments, the brain cell is a cell of the brain stem. In certain embodiments, the brain cell is a cell of the hippocampus. In certain embodiments, the brain cell is a cell of the spinal cord. In certain embodiments, the nucleic acid target is pre-mRNA, mRNA, non-coding RNA, or miRNA. In certain embodiments, the compound is administered to the subject intrathecally.

In certain embodiments, a method of treating or ameliorating a disease, disorder, or symptom thereof in a subject, comprises administering to the subject any of the compounds or compositions provided herein, thereby treating, preventing, or ameliorating a disease, disorder, or symptom in the subject. In certain embodiments, the disease, disorder, or symptom thereof is a central nervous system (CNS) disease, disorder, or symptom thereof. In certain embodiments, the disease, disorder, or symptom thereof is Alzheimer's disease, or a symptom thereof. In certain embodiments, the compound is administered to the subject intrathecally. In certain embodiments, the compound or composition is administered to the subject in a therapeutically effective amount.

In certain embodiment, a compound comprising a TrkB ligand selectively or preferentially targets a cell expressing TrkB compared to a cell not expressing TrkB. In certain embodiments, a compound comprising a TrkB ligand selectively or preferentially targets a cell expressing TrkB compared to a compound not comprising a TrkB ligand.

Also provided herewith is the use of a compound as described herein for the manufacture of a medicament in the treatment of a disease or disorder.

In another aspect, the present disclosure provides methods for making any of the compounds provided herein, comprising one or more compounds and chemical transformations described herein, including Examples 2-29.

### Certain Compounds Comprising an Oligonucleotide

In certain embodiment, compounds described herein comprise oligonucleotides. In certain embodiments, an oligonucleotide has a nucleobase sequence that is at least partially complementary to a target nucleic acid sequence (e.g., an expressed target nucleic acid within a cell). In some embodiments, the oligonucleotide, upon delivery to a cell expressing a target nucleic acid, is able to inhibit the expression of the underlying gene. The gene expression can be inhibited in vitro or in vivo. In certain embodiments, an oligonucleotide comprises one or more ribonucleic acids (e.g., one or more ribonucleosides), deoxyribonucleic acids (e.g., one or more deoxyribonucleosides), modified nucleic acids (e.g., one or more modified nucleobases, sugars, and/or phosphate groups), or a combination thereof. In some embodiments, an oligonucleotide comprises a ribonucleic acid (RNA). In some embodiments, an oligonucleotide comprises a deoxyribonucleic acid (DNA). In some embodiments, an oligonucleotide comprises a modification (e.g., modified nucleobase, modified sugar, or modified phosphate).

In certain embodiments, an oligonucleotide is single-stranded. In some embodiments, a single-stranded oligonucleotide is single-stranded RNA (ssRNA), ssDNA, or a ssRNA/DNA hybrid (e.g., a single-stranded oligonucleotide comprised of both ribonucleosides (modified or unmodified) and deoxyribonucleosides (modified or unmodified)). In some embodiments, an oligonucleotide is double-stranded (e.g., comprised of two single-stranded nucleic acids). Such double-stranded oligonucleotides comprise a first oligonucleotide having a region complementary to a target nucleic acid and a second oligonucleotide having a region complementary to the first oligonucleotide. The first and second oligonucleotides can be independently modified. In certain embodiments the first oligonucleotide is linked to one or more TrkB ligands. In certain embodiments, the second oligonucleotide is linked to one or more TrkB ligands.

In some embodiments, an oligonucleotide is at least 2 (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, or more) nucleotides in length. In some embodiments, an oligonucleotide is at least 5 nucleotides in length. In some embodiments, an oligonucleotide is at least 10 nucleotides in length. In some embodiments, an oligonucleotide is at least 15 nucleotides in length. In some embodiments, an oligonucleotide is at least 16 nucleotides in length. In some embodiments, an oligonucleotide is at least 17 nucleotides in length. In some embodiments, an oligonucleotide is at least 18 nucleotides in length. In some embodiments, an oligonucleotide is at least 19 nucleotides in length. In some embodiments, an oligonucleotide is at least 20 nucleotides in length. In some embodiments, an oligonucleotide is at least 21 nucleotides in length. In some embodiments, an oligonucleotide is at least 22 nucleotides in length. In some embodiments, an oligonucleotide is at least 23 nucleotides in length. In some embodiments, an oligonucleotide is at least 24 nucleotides in length. In some embodiments, an oligonucleotide is at least 25 nucleotides in length. In some embodiments, an oligonucleotide is at least 26 nucleotides in length. In some embodiments, an oligonucleotide is at least 27 nucleotides in length. In some embodiments, an oligonucleotide is at least 28 nucleotides in length. In some embodiments, an oligonucleotide is at least 29 nucleotides in length. In some embodiments, an oligonucleotide is at least 30 nucleotides in length. In some embodiments, an oligonucleotide is at least 40 nucleotides in length. In some embodiments, an oligonucleotide is at least 50 nucleotides in length. In some embodiments, an oligonucleotide is at least 60 nucleotides in length. In some embodiments, an oligonucleotide is at least 70 nucleotides in length. In some embodiments, an oligonucleotide is at least 80 nucleotides in length. In some embodiments, an oligonucleotide is at least 90 nucleotides in length. In some embodiments, an oligonucleotide is at least 100 nucleotides in length. In some embodiments, an oligonucleotide is at least 150 nucleotides in length.

In some embodiments, an oligonucleotide is less than or equal to 150 (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150) nucleotides in length. In some embodiments, an oligonucleotide is less than or equal to 150 nucleotides in length. In some embodiments, an oligonucleotide is less than or equal to 100 nucleotides in length. In some embodiments, an oligonucleotide is less than or equal to 90 nucleotides in length. In some embodiments, an oligonucleotide is less than or equal to 80 nucleotides in length. In some embodiments, an oligonucleotide is less than or equal to 70 nucleotides in length. In some embodiments, an oligonucleotide is less than or equal to 60 nucleotides in length. In some embodiments, an oligonucleotide is less than or equal to 50 nucleotides in length. In some embodiments, an oligonucleotide is less than or equal to 40 nucleotides in length. In some embodiments, an oligonucleotide is less than or equal to 30 nucleotides in length. In some embodiments, an oligonucleotide is less than or equal to 29 nucleotides in length. In some embodiments, an oligonucleotide is less than or equal to 28 nucleotides in length. In some embodiments, an oligonucleotide is less than or equal to 27 nucleotides in length. In some embodiments, an oligonucleotide is less than or equal to 26 nucleotides in length. In some embodiments, an oligonucleotide is less than or equal to 25 nucleotides in length. In some embodiments, an oligonucleotide is less than or equal to 24 nucleotides in length. In some embodiments, an oligonucleotide is less than or equal to 23 nucleotides in length. In some embodiments, an oligonucleotide is less than or equal to 22 nucleotides in length. In some embodiments, an oligonucleotide is less than or equal to 21 nucleotides in length. In some embodiments, an oligonucleotide is less than or equal to 20 nucleotides in length. In some embodiments, an oligonucleotide is less than or equal to 19 nucleotides in length. In some embodiments, an oligonucleotide is less than or equal to 18 nucleotides in length. In some embodiments, an oligonucleotide is less than or equal to 17 nucleotides in length. In some embodiments, an oligonucleotide is less than or equal to 16 nucleotides in length. In some embodiments, an oligonucleotide is less than or equal to 15 nucleotides in length. In some embodiments, an oligonucleotide is less than or equal to 10 nucleotides in length. In some embodiments, an oligonucleotide is less than or equal to 5 nucleotides in length.

In some embodiments, an oligonucleotide is about 5 nucleotides in length to about 150 nucleotides in length. In some embodiments, an oligonucleotide is about 10 nucleotides in length to about 100 nucleotides in length. In some embodiments, an oligonucleotide is about 20 nucleotides in length to about 90 nucleotides in length. In some embodiments, an oligonucleotide is about 30 nucleotides in length to about 80 nucleotides in length. In some embodiments, an oligonucleotide is about 40 nucleotides in length to about 70 nucleotides in length. In some embodiments, an oligonucleotide is about 50 nucleotides in length to about 60 nucleotides in length.

In some embodiments, an oligonucleotide is a therapeutic oligonucleotide. A therapeutic oligonucleotide may comprise, for example, without limitation, a small interfering RNA (siRNA), a microRNA (miRNA) antagonist, a miRNA mimic, an ADAR recruiting molecule, an ADAR targeting molecule, a guide RNA, an antisense oligonucleotide, a short hairpin RNA (shRNA), or combinations thereof.

In certain embodiments, a miRNA is a precursor, primary, and/or mature miRNA.

In certain embodiments, an oligonucleotide comprises or consists of an antisense oligonucleotide. In certain embodiments, an antisense oligonucleotide is complementary to an mRNA. In certain embodiments, an antisense oligonucleotide is complementary to a pre-mRNA. In certain embodiments, an antisense oligonucleotide blocks translation and promotes degradation of the mRNA transcript. In certain embodiments, an antisense oligonucleotide recruits RNase H and promotes degradation of the mRNA transcript. In certain embodiments, an antisense oligonucleotide targets miRNA, inhibiting the miRNA from modulating mRNA expression and promoting degradation of the miRNA.

### Certain Modifications

In certain aspects, the disclosure relates to compounds that comprise oligonucleotides. In certain embodiments, oligonucleotides may be unmodified RNA or DNA or may be modified. In certain embodiments, the oligonucleotides are modified oligonucleotides. In certain embodiments, the modified oligonucleotides comprise at least one modified sugar, modified nucleobase, or modified internucleoside linkage relative to an unmodified RNA or DNA. In certain embodiments, an oligonucleotide has a modified nucleoside. A modified nucleoside may comprise a modified sugar, a modified nucleobase, or both a modified sugar and a modified nucleobase. Modified oligonucleotides may also include end modifications, e.g., 5′-end modifications and 3′-end modifications.

### Sugar Modifications and Motifs

In certain embodiments, a modified sugar is a substituted furanosyl sugar or non-bicyclic modified sugar. In certain embodiments, a modified sugar is a bicyclic or tricyclic modified sugar. In certain embodiments, a modified sugar is a sugar surrogate. A sugar surrogate may comprise one or more substitutions described herein.

In certain embodiments, a modified sugar is a substituted furanosyl or non-bicyclic modified sugar. In certain embodiments, the furanosyl sugar is a ribosyl sugar. In certain embodiments, the furanosyl sugar comprises one or more substituent groups, including, but not limited to, substituent groups at the 2′, 3′, 4′, and 5′ positions.

In certain embodiments, substituents at the 2′ position include, but are not limited to, F and OCH₃ (“OMe”, “O-methyl” or “methoxy”). In certain embodiments, substituent groups at the 2′ position suitable for non-bicyclic modified sugars include, but are not limited to, halo, allyl, amino, azido, SH, CN, OCN, CF₃, OCF₃, F, Cl, Br, SCH₃, SOCH₃, SO₂CH₃, ONO₂, NO₂, N₃, and NH₂. In certain embodiments, substituent groups at the 2′ position include, but are not limited to, O—(C₁-C₁₀) alkoxy, alkoxyalkyl, O-alkyl, S-alkyl, N-alkyl, O-alkenyl, S-alkenyl, N-alkenyl, O-alkynyl, S-alkynyl, N-alkynyl, O-alkyl-O-alkyl, alkynyl, wherein the alkyl, alkenyl and alkynyl can be substituted or unsubstituted C₁ to C₁₀ alkyl or C₂ to C₁₀ alkenyl and alkynyl. In certain embodiments, substituent groups at the 2′ position include, but are not limited to, alkaryl, aralkyl, O-alkaryl, and O-aralkyl. In certain embodiments, these 2′ substituent groups can be further substituted with one or more substituent groups independently selected from hydroxyl, alkoxy, carboxy, benzyl, phenyl, nitro (NO₂), thiol, thioalkoxy, thioalkyl, halogen, alkyl, aryl, alkenyl, and alkynyl. In certain embodiments, substituent groups at the 2′ position include, but are not limited to, O[(CH₂)ₙO]ₘCH₃, O(CH₂)ₙOCH₃, O(CH₂)ₙCH₃, O(CH₂)ₙONH₂, O(CH₂)ₙNH₂, O(CH₂)ₙSCH₃, and O(CH₂)ₙON[(CH₂)ₙCH₃)]₂, where n and m are independently from 1 to about 10. In certain embodiments, substituent groups at the 2′ position include, but are not limited to, OCH₂CH₂OCH₃ (“MOE”), O(CH₂)₂ON(CH₃)₂ (“DMAOE”), O(CH₂)₂O(CH₂)₂N(CH₃)₂ (“DMAEOE”), and OCH₂C(═O)—N(H)CH₃ (“NMA”).

In certain embodiments, substituent groups at the 4′ position suitable for non-bicyclic modified sugars include, but are not limited to, alkoxy (e.g., methoxy), alkyl, and those described in Manoharan et al., WO 2015/106128.

In certain embodiments, substituent groups at the 5′ position suitable for non-bicyclic modified sugars include, but are not limited to, methyl (“Me” or “CH₃”)(R or S), vinyl, and methoxy. In certain embodiments, the 5′ modification is a 5′-monophosphate ((HO)₂(O)P—O-5′); 5′-diphosphate ((HO)₂ (O)P—O—P(HO)(O)—O-5′); 5′-triphosphate ((HO)₂ (O)P—O—(HO)(O)P—O—P(HO)(O)—O-5′); 5′-guanosine cap (7-methylated or non-methylated)(7m-G-O-5′-(HO)(O)P—O—(HO)(O)P—O—P(HO)(O)—O-5′); 5′adenosine cap (Appp), and any modified or unmodified nucleotide cap structure (N—O-5′(HO)(O)P—O—(HO)(O)P—O—P(HO)(O)—O-5′); 5′-monothiophosphate (phosphorothioate; (HO)₂ (S)P—O-5′); 5′-monodithiophosphate (phosphorodithioate; (HO)(HS)(S)P—O-5′), 5′phosphorothiolate ((HO)₂(O)P—S-5′); any additional combination of oxygen/sulfur replaced monophosphate, diphosphate and triphosphates (e.g., 5′-alpha-thiotriphosphate, 5′-gammathiotriphosphate, etc.), 5′-phosphoramidates ((HO)₂ (O)P—NH-5′, (HO)(NH₂)(O)P—O-5′), 5′alkylphosphonates (R=alkyl=methyl, ethyl, isopropyl, propyl, etc., e.g. RP(OH)(O)—O-5′-, 5′alkenylphosphonates (i.e., vinyl, substituted vinyl), (OH)₂(O)P-5′-CH₂—), 5′alkyletherphosphonates (R=alkylether=methoxymethyl (MeOCH₂—), ethoxymethyl, etc., e.g., RP(OH)(O)—O-5′-). In certain embodiments, one or more sugars comprise a 5′-vinylphosphonate modification. In certain embodiments the 5′ modification is at the terminus of an oligonucleotide. In certain embodiments the 5′ modification is at the terminus of an antisense oligonucleotide.

In certain embodiments, substituents described herein for the 2′, 4′, and 5′ position can be added to other specific positions on the sugar. In certain embodiments, such substituents may be added to the 3′ position of the sugar on the 3′ terminal nucleoside or the 5′ position of the 5′ terminal nucleoside. In certain embodiments, a non-bicyclic modified sugar may comprise more than one non-bridging sugar substituent. In certain such embodiments, non-bicyclic modified sugars substituents include, but are not limited to, 5′-Me-2′-F, 5′-Me-2′-OMe (including both R and S isomers). In certain embodiments, modified sugar substituents include those described in Migawa et al., WO 2008/101157 and Rajeev et al., US2013/0203836.

In certain embodiments, a modified sugar is a bicyclic sugar. A bicyclic sugar is a modified sugar comprising two rings, wherein the second ring is formed via a bridge connecting two of the atoms in the first ring, thereby forming a bicyclic structure. In certain embodiments, a bicyclic sugar comprises a bridging substituent that bridges two atoms of the furanosyl ring to form a second ring. In certain embodiments, a bicyclic sugar does not comprise a furanosyl moiety. A “bicyclic nucleoside” (“BNA”) is a nucleoside having a bicyclic sugar. In certain embodiments, the bicyclic sugar comprises a bridge between the 4′ and 2′ furanose ring atoms. In certain embodiments, the bicyclic sugar comprises a bridge between the 5′ and 3′ furanose ring atoms. In certain such embodiments, the furanose ring is a ribose ring. In certain embodiments, 4′ to 2′ bridging substituents include, but are not limited to, 4′—CH₂-2′, 4′-(CH₂)₂-2′, 4′-(CH₂)₃-2′, 4′-CH₂—O-2′ (“LNA”), 4′—CH₂—S-2′, 4′-(CH₂)₂—O-2′ (“ENA”), 4′—CH(CH₃)—O-2′ (“constrained ethyl” or “cEt” when in the S configuration), 4′—CH₂—O—CH₂-2′, 4′—CH₂—N(R)-2′, 4′—CH(CH₂OCH₃)—O-2′ (“constrained MOE” or “cMOE”) and analogs thereof (e.g., U.S. Pat. No. 7,399,845), 4′-C(CH₃)(CH₃)—O-2′ and analogs thereof (e.g., U.S. Pat. No. 8,278,283), 4′—CH₂—N(OCH₃)-2′ and analogs thereof (e.g., U.S. Pat. No. 8,278,425), 4′—CH₂—O—N(CH₃)-2′ (e.g., U.S. Patent Publication No. 2004/0171570), 4′—CH₂—N(R)—O—2′, wherein R is H, C₁-C₁₂ alkyl, or a protecting group (e.g., U.S. Pat. No. 7,427,672), 4′—CH₂—C(H)(CH₃)-2′ (e.g., Chattopadhyaya et al., J. Org. Chem., 2009, 74, 118-134), and 4′—CH₂—C(═CH₂)-2′ and analogs thereof (e.g., U.S. Pat. No. 8,278,426). The entire contents of each of the foregoing are hereby incorporated herein by reference. Additional representative U.S. Patents and U.S. Patent Publications that teach the preparation of bicyclic nucleic acid nucleotides include, but are not limited to, the following: U.S. Pat. Nos. 6,268,490; 6,525,191; 6,670,461; 6,770,748; 6,794,499; 6,998,484; 7,053,207; 7,034,133; 7,084,125; 7,399,845; 7,427,672; 7,569,686; 7,741,457; 8,022,193; 8,030,467; 8,278,425; 8,278,426; 8,278,283; US 2008/0039618; and US 2009/0012281, US 2013/0190383; and WO 2013/036868, the entire contents of each of which are hereby incorporated herein by reference. Any of the foregoing bicyclic nucleosides can be prepared having one or more stereochemical sugar configurations including, for example, α-L-ribofuranose and β-D-ribofuranose (see, e.g., WO 99/14226). Specified bicyclic nucleosides herein are in the β-D configuration, unless otherwise specified.

In certain embodiments, a modified sugar is a sugar surrogate. In certain embodiments, a sugar surrogate has the oxygen atom replaced, e.g., with a sulfur, carbon or nitrogen atom. In certain such embodiments, the sugar surrogate may also comprise bridging and/or non-bridging substituents as described herein. In certain embodiments, sugar surrogates comprise rings having other than 5 atoms. In certain such embodiments, the sugar surrogate comprises a cyclobutyl moiety in place of the pentofuranosyl sugar. In certain embodiments, the sugar surrogate comprises a six membered ring in place of the pentofuranosyl sugar. In certain embodiments, the sugar surrogate comprises a tetrahydropyran (“THP”) in place of the pentofuranosyl sugar. In certain embodiments, the sugar surrogate comprises a morpholino in place of the pentofuranosyl sugar. Representative U.S. Patent Nos that teach the preparation of such modified sugar structures include, but are not limited to, U.S. Pat. Nos. 4,981,957; 5,118,800; 5,166,315; 5,185,444; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,700,920; 7,875,733; 7,939,677, 8,088,904; 8,440,803; and 9,005,906, the entire contents of each of the foregoing are hereby incorporated herein by reference.

In some embodiments, sugar surrogates comprise acyclic moieties. In certain embodiments, the sugar surrogate is an unlocked nucleic acid (“UNA”). A UNA is unlocked acyclic nucleic acid, wherein any of the bonds of the sugar has been removed, forming an unlocked “sugar” residue. In one example, UNA also encompasses a monomer where the bonds between C1′-C4′ have been removed (i.e., the covalent carbon-oxygen-carbon bond between the C1′ and C4′ carbons). In another example, the C2′-C3′ bond (i.e., the covalent carbon-carbon bond between the C2′ and C3′ carbons) of the sugar has been removed. Representative U.S. publications that teach the preparation of UNA include, but are not limited to, U.S. Pat. No. 8,314,227; and U.S. Patent Publication Nos. 2013/0096289; 2013/0011922; and 2011/0313020, the entire contents of each of which are hereby incorporated herein by reference. In certain embodiments, sugar surrogates comprise peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., US2013/130378, the entire contents of which is hereby incorporated herein by reference. Many other bicyclic and tricyclic sugar and sugar surrogate ring systems are known in the art that can be used in modified nucleosides.

In certain aspects, the disclosure relates to compounds comprising at least one oligonucleotide wherein the nucleosides of such oligonucleotide comprise one or more types of modified sugars and/or unmodified sugars arranged along the oligonucleotide or region thereof in a defined pattern or “sugar motif”. In certain instances, such sugar motifs include, but are not limited to, any of the patterns of sugar modifications described herein.

In certain embodiments, an oligonucleotide comprises a gapmer sugar motif. A gapmer oligonucleotide comprises or consists of a region having two external “wing” regions and a central or internal “gap” region. The gap and wing regions form a contiguous sequence of nucleosides, wherein the majority of nucleoside sugars of each of the wings differ from the majority of nucleoside sugars of the gap. In certain embodiments, the wing regions comprise a majority of modified sugars and the gap comprises a majority of unmodified sugars. In certain embodiments, the nucleosides of the gap are deoxynucleosides. Compounds with a gapmer sugar motif are described in, for example, U.S. Pat. No. 8,790,919, the entire contents of which is hereby incorporated herein by reference.

In certain embodiments, one or both oligonucleotides of a double-stranded compound comprise a triplet sugar motif. An oligonucleotide with a triplet sugar motif comprises three identical sugar modifications on three consecutive nucleosides. In certain embodiments, the triplet is at or near the cleavage site of the oligonucleotide. In certain embodiments, an oligonucleotide of a double-stranded compound may contain more than one triplet sugar motif. In certain embodiments, the identical sugar modification of the triplet sugar motif is a 2′-F modification. Compounds with a triplet sugar motif are disclosed, for example, in U.S. Pat. No. 10,668,170, the entire contents of which is incorporated herein by reference.

In certain embodiments, one or both oligonucleotides of a double-stranded compound comprise a quadruplet sugar motif. An oligonucleotide with a quadruplet sugar motif comprises four identical sugar modifications on four consecutive nucleosides. In certain embodiments, the quadruplet is at or near the cleavage site. In certain embodiments, an oligonucleotide of a double-stranded compound may contain more than one quadruplet sugar motif. In certain embodiments, the identical sugar modification of the quadruplet sugar motif is a 2′-F modification. For a double-stranded compound having a duplex region of 19-23 nucleotides in length, the cleavage site of the antisense oligonucleotide is typically around the 10, 11, and 12 positions from the 5′-end. In certain embodiments, the quadruplet sugar motif is at the 8, 9, 10, 11 positions; the 9, 10, 11, 12 positions; the 10, 11, 12, 13 positions; the 11, 12, 13, 14 positions; or the 12, 13, 14, 15 positions of the sense oligonucleotide, counting from the first nucleoside of the 5′-end of the sense oligonucleotide, or, the count starting from the first paired nucleotide within the duplex region from the 5′-end of the sense oligonucleotide. In certain embodiments, the quadruplet sugar motif is at the 8, 9, 10, 11 positions; the 9, 10, 11, 12 positions; the 10, 11, 12, 13 positions; the 11, 12, 13, 14 positions; or the 12, 13, 14, 15 positions of the antisense oligonucleotide, counting from the first nucleoside of the 5′-end of the antisense oligonucleotide, or, the count starting from the first paired nucleotide within the duplex region from the 5′-end of the antisense oligonucleotide. The cleavage site may change according to the length of the duplex region of the double-stranded compound and may change the position of the quadruplet accordingly.

In certain embodiments, an oligonucleotide comprises an alternating sugar motif. In certain embodiments, one or both oligonucleotides of a double-stranded compound comprise an alternating sugar motif. An oligonucleotide with an alternating sugar motif comprises at least two different sugar modifications, wherein one or more consecutive nucleosides comprising a first sugar modification alternates with one or more consecutive nucleosides comprising a second sugar modification, and one or more consecutive nucleosides comprising a third sugar modification, etc. For example, if A, B, and C each represent one type of modification to the nucleoside, the alternating motif can be “ABABABABABAB . . . ,” “AABBAABBAABB . . . ,” “AABAABAABAAB “AAABAAABAAAB . . . ,” “AAABBBAAABBB . . . ,” or “ABCABCABCABC . . . ” etc. In certain embodiments, the alternating sugar motif is repeated for at least 2, 3, 4, 5, 6, 7, 8, 9, 10, 11, 12, 13, 14, 15, 16, 17, 18, 19, 20, 21, 22, or 23 contiguous nucleobases along an oligonucleotide. In certain embodiments, the alternating sugar motif is comprised of two different sugar modifications. In certain embodiments, the alternating sugar motif comprises 2′—OMe and 2′-F sugar modifications.

In certain embodiments, each nucleoside of an oligonucleotide is independently modified with one or more sugar modifications provided herein. In certain embodiments, each oligonucleotide of a double-stranded compound independently has one or more sugar motifs provided herein. In certain embodiments, an oligonucleotide containing a sugar motif is fully modified in that each nucleoside other than the nucleosides comprising the sugar motif comprises a sugar modification.

### Nucleobase Modifications and Motifs

In certain embodiments, modified oligonucleotides comprise one or more nucleosides comprising a modified nucleobase. In certain embodiments, modified oligonucleotides comprise one or more nucleosides that do not comprise a nucleobase, referred to as an abasic nucleoside.

In certain embodiments, modified nucleobases are selected from: 5-substituted pyrimidines, 6-azapyrimidines, alkyl or alkynyl substituted pyrimidines, alkyl substituted purines, and N-2, N-6 and O-6 substituted purines. In certain embodiments, modified nucleobases are selected from: 2-aminopropyladenine, 5-hydroxymethyl cytosine, 5-methylcytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly, 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size expanded bases, and fluorinated bases. Further modified nucleobases include tricyclic pyrimidines, such as 1,3-diazaphenoxazine-2-one, 1,3-diazaphenothiazine-2-one, and 9-(2-aminoethoxy)-1,3-diazaphenoxazine-2-one (G-clamp). Modified nucleobases may also include those in which the purine or pyrimidine base is replaced with other heterocycles, for example, 7-deaza-adenine, 7-deazaguanosine, 2-aminopyridine and 2-pyridone.

Further nucleobases include those disclosed in U.S. Pat. No. 3,687,808; Modified Nucleosides in Biochemistry, Biotechnology and Medicine, Herdewijn, P. ed. Wiley-VCH, 2008; The Concise Encyclopedia Of Polymer Science And Engineering, pages 858-859; Kroschwitz, J. L., Ed., John Wiley & Sons, 1990, 858-859; Englisch et al., Angewandte Chemie, International Edition, 1991, 30, 613; Sanghvi, Y. S., Chapter 15, dsRNA Research and Applications, pages 289-302; Antisense Research and Applications, Crooke, S. T. and Lebleu, B., Eds., CRC Press, 1993, 273-288; Antisense Drug Technology, Crooke S. T., Ed., CRC Press, 2008, 163-166 and 442-443 (Chapters 6 and 15), each of which are hereby incorporated herein by reference.

Publications that teach the preparation of certain of the above noted modified nucleobases, as well as other modified nucleobases include without limitation, U.S. Patent Publication Nos. 2003/0158403 and 2003/0175906; U.S. Pat. Nos. 4,845,205; 5,130,302; 5,134,066; 5,175,273; 5,367,066; 5,432,272; 5,434,257; 5,457,187; 5,459,255; 5,484,908; 5,502,177; 5,525,711; 5,552,540; 5,587,469; 5,594,121; 5,596,091; 5,614,617; 5,645,985; 5,681,941; 5,811,534; 5,750,692; 5,948,903; 5,587,470; 5,457,191; 5,763,588; 5,830,653; 5,808,027; 6,005,096, 6,015,886; 6,147,200; 6,166,197; 6,166,199; 6,222,025; 6,235,887; 6,380,368; 6,528,640; 6,639,062; 6,617,438; 7,045,610; 7,427,672; and 7,495,088, the entire contents of each of which are hereby incorporated herein by reference.

In certain embodiments, oligonucleotides comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methylcytosines.

In certain embodiments, modified oligonucleotides comprise a block of modified nucleobases. In certain such embodiments, the block is at the 3′-end of the oligonucleotide. In certain embodiments, the block is within 3 nucleosides of the 3′-end of the oligonucleotide. In certain embodiments, the block is at the 5′-end of the oligonucleotide. In certain embodiments, the block is within 3 nucleosides of the 5′-end of the oligonucleotide.

### Internucleoside Linkage Modifications and Motifs

A 3′ to 5′ phosphodiester linkage is the naturally occurring internucleoside linkage of RNA and DNA. In certain embodiments, an oligonucleotide has one or more modified, i.e., non-naturally occurring, internucleoside linkages. Certain non-naturally occurring internucleoside linkages may impart desirable properties such as, for example, enhanced cellular uptake, enhanced affinity for target nucleic acids, and increased stability in the presence of nucleases. Representative phosphorus-containing modified internucleoside linkages include, but are not limited to, phosphotriesters, alkylphosphonates (e.g., methylphosphonates), phosphoramidates, and phosphorothioates (“P═S”), and phosphorodithioates (“HS—P═S”). Representative non-phosphorus containing internucleoside linking groups include, but are not limited to, methylenemethylimino (—CH₂—N(CH₃)—O—CH₂), thiodiester, thionocarbamate (—O—C(═O)(NH)—S—); siloxane (—O—SiH₂—O—); and N,N′-dimethylhydrazine (—CH₂—N((CH₃)—N((CH₃)—). Methods of preparation of phosphorous-containing and non-phosphorous-containing internucleoside linkages are well known to those skilled in the art. Neutral internucleoside linkages include, without limitation, phosphotriesters, methylphosphonates, MMI (3′—CH₂—N(CH₃)—O-5′), amide-3 (3′—CH₂—C(═O)—N(H)-5′), amide-4 (3′—CH₂—N(H)—C(═O)-5′), formacetal (3′—O—CH₂—O-5′), methoxypropyl, and thioformacetal (3′—S—CH₂—O-5′). Further neutral internucleoside linkages include nonionic linkages comprising siloxane (dialkylsiloxane), carboxylate ester, carboxamide, sulfide, sulfonate ester and amides (See, for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

In certain embodiments, an oligonucleotide comprises at least one modified internucleoside linkage. A modified internucleoside linkage may be placed at any position of an oligonucleotide. For double-stranded compounds, a modified internucleoside linkage may be placed within the sense oligonucleotide, antisense oligonucleotide, or both oligonucleotides of the double-stranded compound.

In certain embodiments, the internucleoside linkage modification may occur on every nucleoside of an oligonucleotide. In certain embodiments, internucleoside linkage modifications may occur in an alternating pattern along an oligonucleotide. In certain embodiments, essentially each internucleoside linking group is a phosphate internucleoside linkage (P═O). In certain embodiments, each internucleoside linking group of a modified oligonucleotide is a phosphorothioate (P═S). In certain embodiments, each internucleoside linking group of a modified oligonucleotide is independently selected from a phosphorothioate and phosphate internucleoside linkage. In certain embodiments, the pattern of the internucleoside linkage modification on each oligonucleotide of a double-stranded compound is the same. In certain embodiments, the pattern of the internucleoside linkage modification on each oligonucleotide of a double-stranded compound is different. In certain embodiments, a double-stranded compound comprises 6-8 modified internucleoside linkages. In certain embodiments, the 6-8 modified internucleoside linkages are phosphorothioate internucleoside linkages or alkylphosphonate internucleoside linkages. In certain embodiments, the sense oligonucleotide comprises at least two modified internucleoside linkages at either or both the 5′-end and the 3′-end. In certain such embodiments, the modified internucleoside linkages are phosphorothioate internucleoside linkages or alkylphosphonate internucleoside linkages. In certain embodiments, the antisense oligonucleotide comprises at least two modified internucleoside linkages at either or both the 5′-end and the 3′-end. In certain such embodiments, the modified internucleoside linkages are phosphorothioate internucleoside linkages or alkylphosphonate internucleoside linkages.

In certain embodiments, a double-stranded compound comprises an overhang region. In certain embodiments, a double-stranded compound comprises a phosphorothioate or alkylphosphonate internucleoside linkage modification in the overhang region. In certain embodiments, a double-stranded compound comprises a phosphorothioate or alkylphosphonate internucleotide linkage linking the overhang nucleotide with a paired nucleotide that is next to the overhang nucleotide. For instance, there may be at least two phosphorothioate internucleoside linkages between the terminal three nucleosides, in which two of the three nucleosides are overhang nucleosides, and the third is a paired nucleoside next to the overhang nucleoside. These terminal three nucleosides may be at the 3′-end of the antisense oligonucleotide, the 3′-end of the sense oligonucleotide, the 5′-end of the antisense oligonucleotide, or the 5′-end of the antisense oligonucleotide.

In certain embodiments, modified oligonucleotides comprise one or more internucleoside linkages having chiral centers. Representative chiral internucleoside linkages include, but are not limited to, alkylphosphonates and phosphorothioates. Modified oligonucleotides comprising internucleoside linkages having chiral centers can be prepared as populations of modified oligonucleotides comprising stereorandom internucleoside linkages, or as populations of modified oligonucleotides comprising phosphorothioate linkages in particular stereochemical configurations. In certain embodiments, populations of modified oligonucleotides comprise phosphorothioate internucleoside linkages wherein all of the phosphorothioate internucleoside linkages are stereorandom. Such modified oligonucleotides can be generated using synthetic methods that result in random selection of the stereochemical configuration of each phosphorothioate linkage. As is well understood by those of skill in the art, each individual phosphorothioate of each individual oligonucleotide molecule has a defined stereoconfiguration. In certain embodiments, populations of modified oligonucleotides are enriched for modified oligonucleotides comprising one or more particular phosphorothioate internucleoside linkages in a particular, independently selected stereochemical configuration. In certain embodiments, the particular configuration of the particular phosphorothioate linkage is present in at least 65% of the molecules in the population. In certain embodiments, the particular configuration of the particular phosphorothioate linkage is present in at least 70% of the molecules in the population. In certain embodiments, the particular configuration of the particular phosphorothioate linkage is present in at least 80% of the molecules in the population. In certain embodiments, the particular configuration of the particular phosphorothioate linkage is present in at least 90% of the molecules in the population. In certain embodiments, the particular configuration of the particular phosphorothioate linkage is present in at least 99% of the molecules in the population. Such enriched populations of modified oligonucleotides can be generated using synthetic methods known in the art, e.g., methods described in Oka et al., JACS 125, 8307 (2003), Wan et al. Nuc. Acid. Res. 42, 13456 (2014), and WO 2017/015555. In certain embodiments, a population of modified oligonucleotides is enriched for modified oligonucleotides having at least one indicated phosphorothioate in the (Sp) configuration. In certain embodiments, a population of modified oligonucleotides is enriched for modified oligonucleotides having at least one phosphorothioate in the (Rp) configuration.

### TrkB Ligands

In some embodiments, the compounds provided herein comprise a tropomyosin receptor kinase B (TrkB, also known as tyrosine receptor kinase B) ligand. In some embodiments, a TrkB ligand is useful for directing a therapeutic, prophylactic, or diagnostic agent. In certain embodiments, a therapeutic agent is an oligonucleotide (e.g., a therapeutic oligonucleotide). In some embodiments, a TrkB ligand directs an oligonucleotide to a locality. In some embodiments, a TrkB ligand targets tissues. In some embodiments, the tissue is brain tissue. In some embodiments, a TrkB ligand targets a cell receptor. In some embodiments, a cell receptor is TrkB. In some embodiments, a TrkB receptor is in the brain. In some embodiments, a TrkB receptor is in the frontal cortex. In some embodiments, a TrkB receptor is in the striatum. In some embodiments, a TrkB receptor is in the cerebellum. In some embodiments, a TrkB receptor is in the brain stem. In some embodiments, a TrkB receptor is in the hippocampus. In some embodiments, a TrkB receptor is in the spinal cord.

The use of any TrkB ligand in the compounds provided herein is contemplated by the present disclosure. TrkB ligands are known in the art, and a person of ordinary skill in the art would be capable of identifying additional TrkB ligands for use in the compounds described herein beyond those explicitly provided by the present disclosure. The present disclosure also contemplates the use of derivatives of any TrkB ligand provided herein or known in the art in the presently described compounds, and a person of ordinary skill in the art would know how to make such derivatives.

In some embodiments, a TrkB ligand is a TrkB agonist. In some embodiments, a TrkB ligand is a TrkB antagonist. Exemplary TrkB ligands for use in the present disclosure include, but are not limited to, any of the following TrkB ligands, and derivatives thereof:

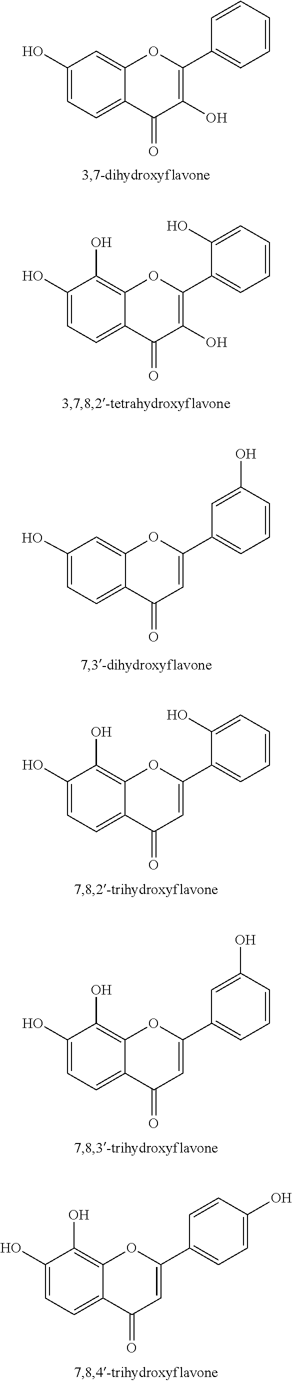 [SMILES: O=c1c(O)c(-c2ccccc2)oc2cc(O)ccc12 ; O=c1c(O)c(-c2ccccc2O)oc2c(O)c(O)ccc12 ; O=c1cc(-c2cccc(O)c2)oc2cc(O)ccc12 ; O=c1cc(-c2ccccc2O)oc2c(O)c(O)ccc12 ; O=c1cc(-c2cccc(O)c2)oc2c(O)c(O)ccc12 ; O=c1cc(-c2ccc(O)cc2)oc2c(O)c(O)ccc12]

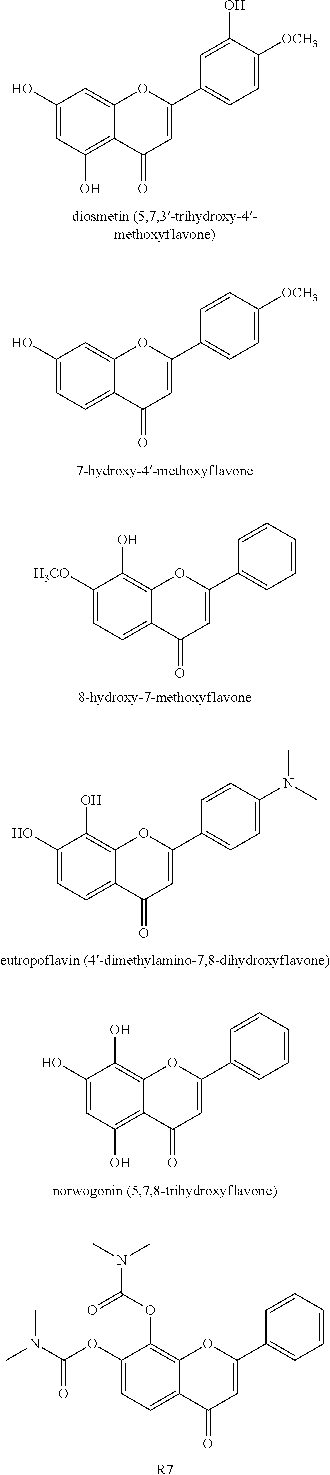

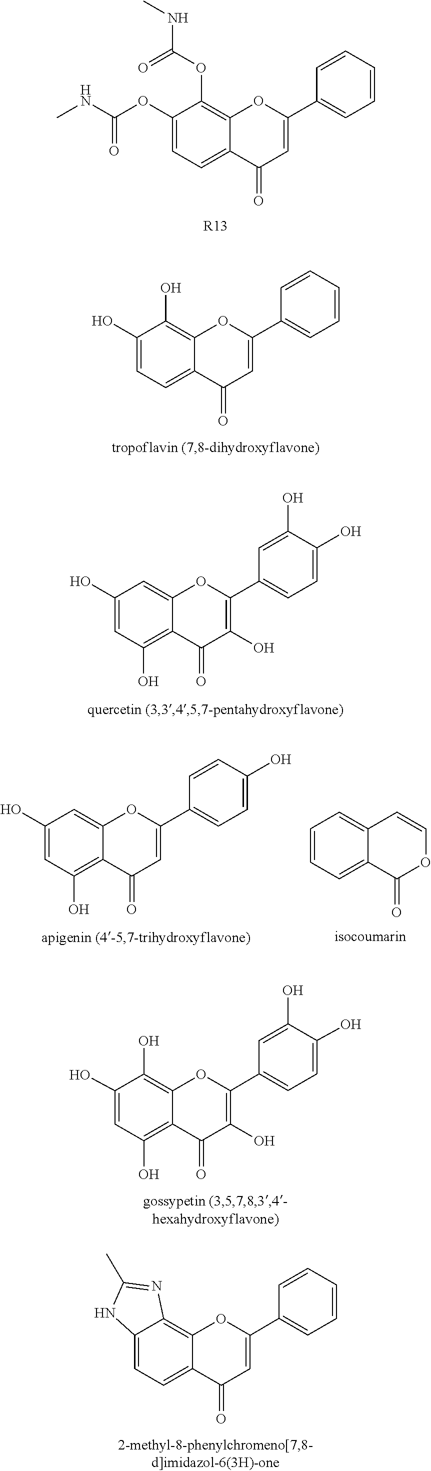

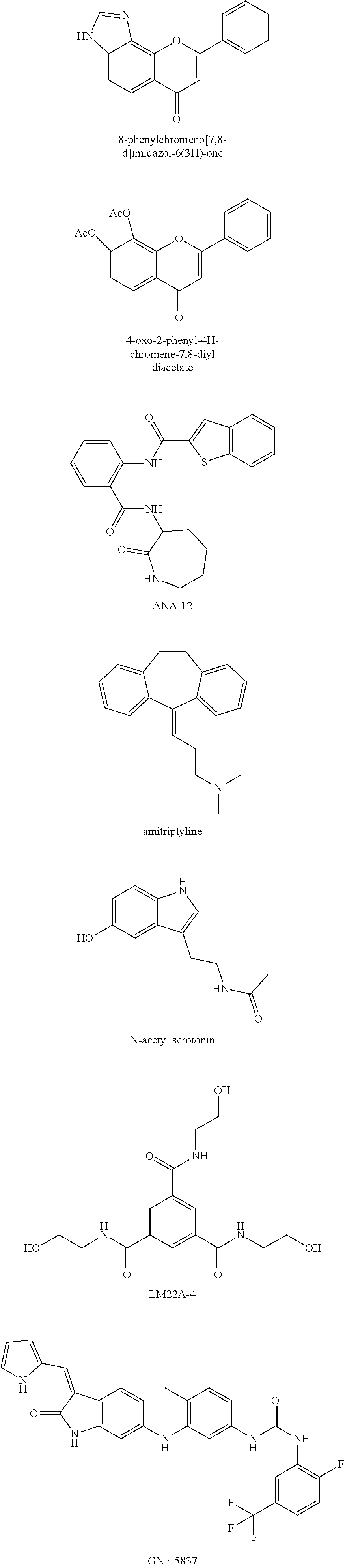

In some embodiments, a TrkB ligand is an aptamer. For example, aptamers that are TrkB agonists are described in Huang, Y. Z. et al. RNA Aptamer-Based Functional Ligands of the Neurotrophin Receptor, TrkB. Mol. Pharmacol. 2012, 82 (4), 623-635.

In certain embodiments, a TrkB ligand is a flavone, a tropoflavin, or a prodrug or derivative thereof. In some embodiments, a TrkB ligand comprises the structure:

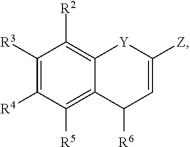

wherein R² is hydrogen, —OH, —O-alkyl, —O-alkenyl, —NH₂, or —SH; R³ is hydrogen, —OH, —O-alkyl, —O-alkenyl, —NH₂, or —SH; R⁴ is hydrogen, —OH, —O-alkyl, —NH₂, or —SH; R⁵ is hydrogen, —OH, —O-alkyl, —NH₂, or —SH; R⁶ is hydrogen, —OH, —O-alkyl, —NH₂, —SH, or ═O; Y is CH₂, NH, S, or O; and Z is optionally substituted aryl or optionally substituted heteroaryl.

In certain embodiments, a TrkB ligand is ANA-12. In some embodiments, a TrkB ligand is an anti-TrkB antibody. In certain embodiments, a TrkB ligand is an anti-TrkB antibody fragment, or an anti-TrkB antibody variant. An “anti-TrkB antibody” refers to an immune system protein that recognizes, binds to, or otherwise interacts with a TrkB receptor.

In certain embodiments, a TrkB ligand is conjugated (e.g., linked, connected, attached, associated with) to and agent moiety. In certain embodiments, the agent moiety is a therapeutic, prophylactic, diagnostic, or imaging agent. In certain embodiments, the agent is a small molecule or oligomeric compound. In certain embodiments, the agent moiety is protein, peptide, antibody, oligonucleotide, small molecule, large molecule, or combination thereof.

In some embodiments, more than one TrkB ligand is conjugated to an agent moiety. In some embodiments, at least two TrkB ligands (e.g., 2, 3, 4, 5, 6, 7, 8, 9, 10, or more TrkB ligands) are conjugated to an agent moiety. In some embodiments, two TrkB ligands are conjugated to an agent moiety. In some embodiments, three TrkB ligands are conjugated to an agent moiety. In some embodiments, four TrkB ligands are conjugated to an agent moiety. In some embodiments, five TrkB ligands are conjugated to an agent moiety. In some embodiments, more than five TrkB ligands are conjugated to an agent moiety. In some embodiments, at least one to about five TrkB ligands are conjugated to an agent moiety. In some embodiments, at least one to about four TrkB ligands are conjugated to an agent moiety. In some embodiments, at least one to about three TrkB ligands are conjugated to an agent moiety. In some embodiments, at least one to about two TrkB ligands are conjugated to an agent moiety.

When an agent moiety is conjugated to multiple TrkB ligands, all of the TrkB ligands may be conjugated at or near the same position on the agent moiety, or the TrkB ligands may be conjugated to multiple different positions on the agent moiety.

In some embodiments, an oligonucleotide is conjugated (e.g., connected, attached, associated with) to a TrkB ligand through either a 5′ end and/or a 3′ end of the oligonucleotide, or at an internal position in an oligonucleotide (i.e., at a nucleotide on the oligonucleotide other than the 5′ or 3′ nucleotide). In some embodiments, an oligonucleotide is conjugated to a TrkB ligand through the 5′ end of the oligonucleotide. In some embodiments, an oligonucleotide is conjugated to a TrkB ligand through the 3′ end of the oligonucleotide. In some embodiments, an oligonucleotide is conjugated to TrkB ligands through both the 5′ end and the 3′ end of the oligonucleotide. In some embodiments, an oligonucleotide is conjugated to a TrkB ligand at an internal position within the oligonucleotide (e.g., in an “internally-modified oligonucleotide”).

In some embodiments, an oligonucleotide is conjugated to more than one TrkB ligand. In some embodiments, an oligonucleotide is conjugated to at least two TrkB ligands (e.g., 2, 3, 4, 5, 6, 7, 8, 9, 10, or more TrkB ligands). In some embodiments, an oligonucleotide is conjugated to two TrkB ligands. In some embodiments, an oligonucleotide is conjugated to three TrkB ligands. In some embodiments, an oligonucleotide is conjugated four TrkB ligands. In some embodiments, an oligonucleotide is conjugated to five TrkB ligands. In some embodiments, an oligonucleotide is conjugated to more than five TrkB ligands. In some embodiments, an oligonucleotide is conjugated to at least one to about five TrkB ligands. In some embodiments, an oligonucleotide is conjugated to at least one to about four TrkB ligands. In some embodiments, an oligonucleotide is conjugated to at least one to about three TrkB ligands. In some embodiments, an oligonucleotide is conjugated to at least one to about two TrkB ligands.

When an oligonucleotide is conjugated to multiple TrkB ligands, all of the TrkB ligands may be conjugated at or near the same position on the oligonucleotide, or the TrkB ligands may be conjugated to multiple different positions on the oligonucleotide. In some embodiments, multiple TrkB ligands (i.e., two, three, four, five, or more TrkB ligands) are conjugated at the 5′ end of the oligonucleotide. In some embodiments, multiple TrkB ligands (i.e., two, three, four, five, or more TrkB ligands) are conjugated at the 3′ end of the oligonucleotide. In some embodiments, multiple TrkB ligands (i.e., two, three, four, five, or more TrkB ligands) are conjugated at one or more internal positions of the oligonucleotide. In some embodiments, an oligonucleotide is conjugated to one or more TrkB ligands at the 5′ end of the oligonucleotide and/or one or more TrkB ligands at the 3′ end of the oligonucleotide and/or one or more TrkB ligands at an internal position, or multiple internal positions, of the oligonucleotide.

### Linkers

In certain embodiments, conjugates of the compound formulae described herein are provided. In certain embodiments, the conjugates comprise a TrkB ligand covalently coupled to an agent moiety. In certain embodiments, the conjugates provided herein comprise one or more linker moieties. In certain embodiments, the one or more linker moieties link a TrkB ligand to an agent moiety. In certain embodiments, the agent moiety is a protein, peptide, antibody, nucleic acid, small molecule, large molecule, therapeutic, prophylactic, diagnostic, or imaging agent. In some embodiments, a compound is conjugated to an oligonucleotide. In certain embodiments, a TrkB ligand is conjugated to an oligonucleotide. In certain embodiments, a compound comprises one or more TrkB ligands, one or more linker moieties, and one or more agent moieties, wherein the TrkB ligands are conjugated (e.g., linked, connected, attached, associated with) to the one of more agent moieties through one or more linker moieties.

Conjugates as disclosed herein can be manufactured using any available method. When associating compounds provided herein with agent moieties (e.g., a TrkB ligand with an oligonucleotide), the moieties may be linked directly or indirectly (e.g., through a linker moiety; that is, the linker is covalently bonded to each of the oligonucleotide and the TrkB ligand; in some formulae herein “-Lₙ-” wherein n is a number (e.g., L₁, L₂, L₃, and L₄). For example, the oligonucleotide and TrkB ligand may be directly associated with one another, e.g., by one or more covalent bonds, or may be associated by means of one or more linkers. A “linker” refers to any chemical moiety (e.g., a combination of atoms having appropriate valency according to known chemistry principles) used to conjugate two components of the compounds provided herein (e.g., a TrkB ligand and an oligonucleotide) to one another. Each of the two components may be connected to any portion of any of the linkers provided herein. In some embodiments, one component of the compounds provided herein (e.g., a TrkB ligand or an oligonucleotide) is connected by a bond to one end of a linker, and the other component is connected by a bond to the other end of the linker. In some embodiments, one or both components of the compounds provided herein may be connected by a bond to an internal position within any of the linkers described herein. For example, in the context of an “alkyl linker,” a TrkB ligand may be joined by a bond to a carbon at one end of the alkyl linker, and an oligonucleotide may be joined by a bond to a carbon at the other end of the alkyl linker. In some embodiments, a linker is optionally substituted, and a TrkB ligand or another linker may be joined by a bond to the optional substituent.

In certain embodiments, the compounds provided herein comprise one or more linking groups. In certain embodiments, each of L₁, L₂, L₃, and L₄ comprises a linking group. In certain embodiments, a linking group is covalently bound to a TrkB ligand. In certain embodiments, a linking group is covalently bound to an oligonucleotide. In certain embodiments, a linking group is covalently bound to a cleavable moiety. In certain embodiments, a linking group comprises a cleavable bond. In certain embodiments, a linking group does not comprise a cleavable moiety. In certain embodiments, a linking group comprises a covalent attachment to a solid support. In certain embodiments, a linking group includes multiple positions for attachment of TrkB ligands.

In certain embodiments, a linking group comprises a chain structure, such as a hydrocarbyl chain, or an oligomer of repeating units or combination of such repeating units. In certain embodiments, a linking group comprises 1 to 50 repeating units, 1 to 40 repeating units, 1 to 25 repeating units, 1 to 20 repeating units, 1 to 15 repeating units, 1 to 10 repeating units, or 1 to 5 repeating units. In certain embodiments, a linking group is 1 to 50 atoms long, 1 to 40 atoms long, 1 to 25 atoms long, 1 to 20 atoms long, 1 to 15 atoms long, 1 to 10 atoms long, or 1 to 5 atoms long.

In certain embodiments, a linking group contains carbon atoms. In certain embodiments, a linking group contains heteroatoms (e.g., nitrogen, oxygen, sulfur, etc.). In certain embodiments, a linking group forms amide linkages, ester linkages, or disulfide linkages. In certain embodiments, a linking group forms hydrazone linkages, oxime linkages, imine linkages, guanidine linkages, urea linkages, carbamate linkages, unsaturated alkyl linkages, sulfonamide linkages or 4-8 membered hetero cyclic linkages. In certain embodiments, a linking group comprises one or more groups selected from alkyl, amino, oxo, amide, disulfide, polyethylene glycol, ether, thioether, and hydroxylamino. In certain embodiments, a linking group comprises at least one phosphorus group. In certain embodiments, a linking group comprises at least one phosphate group. In certain embodiments, a linking group includes at least one neutral linking group. In certain embodiments, a linking group is substituted with various substituents including, but not limited to, hydrogen atoms, alkyl, alkenyl, alkynyl, amino, alkylamino, dialkylamino, trialkylamino, hydroxyl, alkoxy, halogen, aryl, heterocyclic, aromatic heterocyclic, cyano, amide, carbamoyl, carboxylic acid, ester, thioether, alkylthioether, thiol, and ureido groups. As would be appreciated by one of skill in this art, each of these groups may in turn be substituted.

In certain embodiments, a linking group includes, but is not limited to, substituted or unsubstituted C₁-C₁₀ alkyl, substituted or unsubstituted C₂-C₁₀ alkenyl, or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl, and alkynyl. In certain embodiments, a linking group is an aliphatic or heteroaliphatic. For example, the linking group can a polyalkyl linking group. The linking group can be a polyether linking group. The linking group can be a polyethylene linking group, such as PEG.

In certain embodiments, the linking group is a short peptide chain. In certain embodiments, a linking group comprises 1 to 40 amino acids, 1 to 25 amino acids, 1 to 20 amino acids, 1 to 15 amino acids, 1 to 10 amino acids, or 1 to 5 amino acids.

In certain embodiments, a linking group comprises linker-nucleosides. In certain embodiments, a linking group comprises 1 to 40 linker-nucleosides, 1 to 25 linker-nucleosides, 1 to 20 linker-nucleosides, 1 to 15 linker-nucleosides, 1 to 10 linker-nucleosides, or 1 to 5 linker-nucleosides. In certain embodiments, such linker-nucleosides may be modified or unmodified nucleosides. It is typically desirable for linker-nucleosides to be cleaved from the compound after it reaches a target tissue. Accordingly, linker-nucleosides herein can be linked to one another and to the remainder of the compound through cleavable bonds. Herein, linker-nucleosides are not considered to be part of an oligonucleotide payload. Accordingly, in embodiments in which a compound comprises an oligonucleotide consisting of a specified number or range of linked nucleosides and/or a specified percent complementarity to a reference nucleic acid and the compound also comprises a TrkB ligand comprising a linking group comprising linker-nucleosides, those linker-nucleosides are not counted toward the length of the oligonucleotide and are not used in determining the percent complementarity of the oligonucleotide for the reference nucleic acid.

In certain embodiments, the linking group includes a protein binding group. In certain embodiments, the protein binding group is a lipid such as for example including but not limited to cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine), a vitamin (e.g., folate, vitamin A, vitamin E, biotin, pyridoxal), a peptide, a carbohydrate (e.g., monosaccharide, disaccharide, trisaccharide, tetrasaccharide, oligosaccharide, polysaccharide), an endosomolytic component, a steroid (e.g., uvaol, hecigenin, diosgenin), a terpene (e.g., triterpene, e.g., sarsasapogenin, friedelin, epifriedelanol derivatized lithocholic acid), or a cationic lipid. In certain embodiments, the protein binding group is a C₁₆ to C₂₂ long chain saturated or unsaturated fatty acid, cholesterol, cholic acid, vitamin E, adamantane or 1-pentafluoropropyl.

In certain embodiments, a linking group includes, but is not limited to, pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA).

In certain embodiments, a linking group includes, without limitation, those linking groups described in the following references: U.S. Pat. Nos. 5,994,517; 6,300,319; 6,660,720; 6,906,182; 7,262,177; 7,491,805; 8,106,022; 7,723,509; 9,127,276; US 2006/0148740; US 2011/0123520; WO2013/033230; WO2012/037254, Biessen et al., J. Med. Chem. 1995, 38, 1846-1852; Lee et al., Bioorganic & Medicinal Chemistry 2011,19, 2494-2500; Rensen et al., J. Biol. Chem. 2001, 276, 37577-37584; Rensen et al., J. Med. Chem. 2004, 47, 5798-5808; Sliedregt et al., J. Med. Chem. 1999, 42, 609-618; Valentijn et al., Tetrahedron, 1997, 53, 759-770; Lee, Carhohydr Res, 1978, 67, 509-514; Connolly et al., J Biol Chem, 1982, 257, 939-945; Pavia et al., Int J Pep Protein Res, 1983, 22, 539-548; Lee et al., Biochem, 1984, 23, 4255-4261; Lee et al., Glycoconjugate J, 1987, 4, 317-328; Toyokuni et al., Tetrahedron Lett, 1990, 31, 2673-2676; Biessen et al., J Med Chem, 1995, 38, 1538-1546; Valentijn et al., Tetrahedron, 1997, 53, 759-770; Kim et al., Tetrahedron Lett, 1997, 38, 3487-3490; Lee et al., Bioconjug Chem, 1997, 8, 762-765; Kato et al., Glycobiol, 2001, 11, 821-829; Rensen et al., J Biol Chem, 2001, 276, 37577-37584; Lee et al., Methods Enzymol, 2003, 362, 38-43; Westerlind et al., Glycoconj J, 2004, 21, 227-241; Lee et al., Bioorg Med Chem Lett, 2006, 16 (19), 5132-5135; Maierhofer et al., Bioorg Med Chem, 2007, 15, 7661-7676; Khorev et al., Bioorg Med Chem, 2008, 16, 5216-5231; Lee et al., Bioorg Med Chem, 2011, 19, 2494-2500; Kornilova et al., Analyt Biochem, 2012, 425, 43-46; Pujol et al., Angew Chemie Int Ed Engl, 2012, 51, 7445-7448; Biessen et al., J Med Chem, 1995, 38, 1846-1852; Sliedregt et al., J Med Chem, 1999, 42, 609-618; Rensen et al., J Med Chem, 2004, 47, 5798-5808; Rensen et al., Arterioscler Thromh Vase Biol, 2006, 26, 169-175; van Rossenberg et al., Gene Ther, 2004, 11, 457-464; Sato et al., JAm Chem Soc, 2004, 126, 14013-14022; Lee et al., J Org Chem, 2012, 77, 7564-7571; Biessen et al., FASEB J, 2000, 14, 1784-1792; Rajur et al., Bioconjug Chem, 1997, 8, 935-940; Duff et al., Methods Enzymol, 2000, 313, 297-321; Maier et al., Bioconjug Chem, 2003, 14, 18-29; Jayaprakash et al., Org Lett, 2010, 12, 5410-5413; Manoharan, Antisense Nucleic Acid Drug Dev, 2002, 12, 103-128; Merwin et al., Bioconjug Chem, 1994, 5, 612-620; Tomiya et al., Bioorg Med Chem, 2013, 21, 5275-5281; International applications WO1998/013381; WO2011/038356; WO1997/046098; WO2008/098788; WO2004/101619; WO2012/037254; WO2011/120053; WO2011/100131; WO2011/163121; WO2012/177947; WO2013/033230; WO2013/075035; WO2012/083185; WO2012/083046; WO2009/082607; WO2009/134487; WO2010/144740; WO2010/148013; WO1997/020563; WO2010/088537; WO2002/043771; WO2010/129709; WO2012/068187; WO2009/126933; WO2004/024757; WO2010/054406; WO2012/089352; WO2012/089602; WO2013/166121; WO2013/165816; U.S. Pat. Nos. 4,751,219; 7,582,744; 8,552,163; 8,137,695; 6,908,903; 6,383,812; 7,262,177; 6,525,031; 5,994,517; 6,660,720; 6,300,319; 7,723,509; 8,106,022; 7,491,805; 7,491,805; 8,541,548; 8,344,125; 8,313,772; 8,349,308; 8,450,467; 8,501,930; 8,158,601; 7,262,177; 6,906,182; 6,620,916; 8,435,491; 8,404,862; 7,851,615; Published U.S. Patent Application Publications US2011/0097264; US2011/0097265; US2013/0004427; US2003/0119724; US2011/0207799; US2012/0035115; US2012/0230938; US2005/0164235; US2006/0183886; US2012/0136042; US2012/0095075; US2013/0109817; US2006/0148740; US2008/0206869; US2012/0165393; US2012/0101148; US2013/0121954; US2011/0123520; US2003/0077829; US2008/0108801; and US2009/0203132; each of which is incorporated herein by reference in its entirety.

In certain embodiments, L₁, L₂, L₃, and L₄ independently comprise or together comprise a structure selected from among:

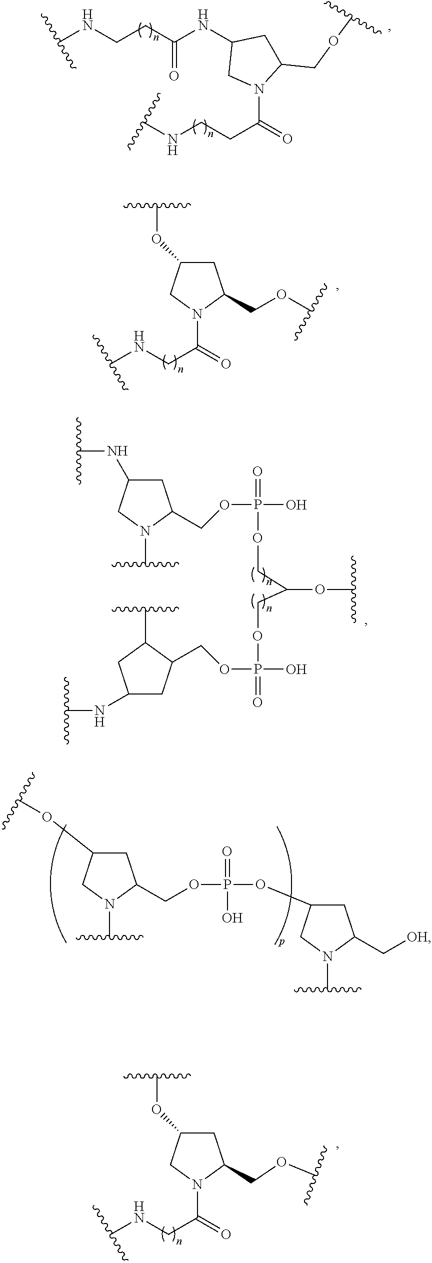

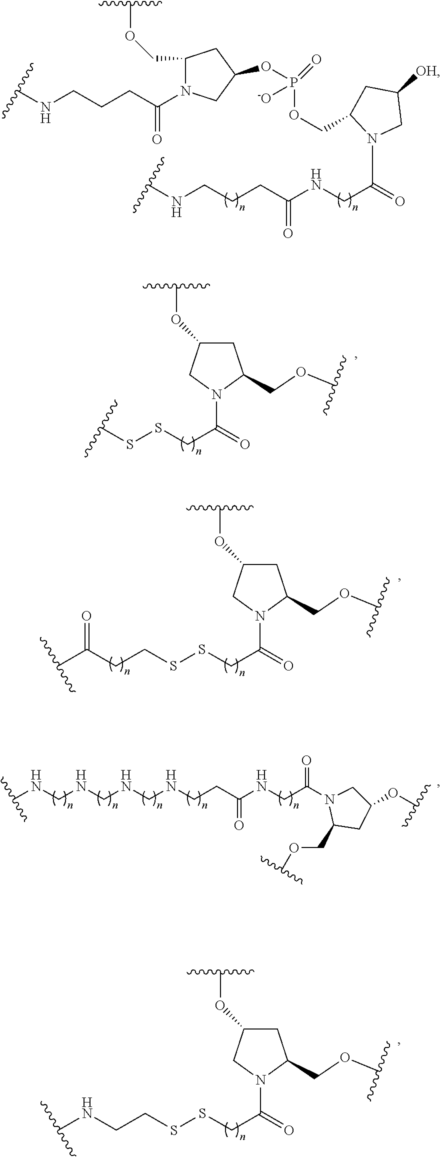

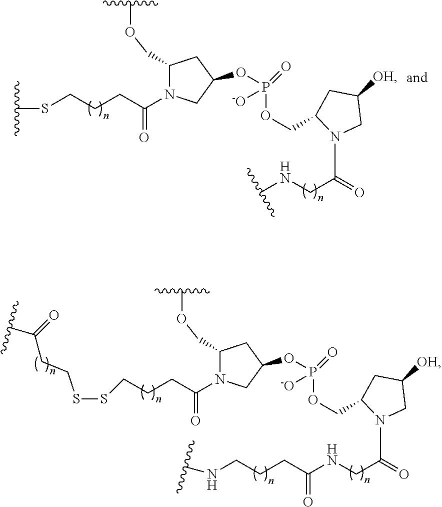

wherein each n is, independently, from 1 to 20 (i.e., 1, 2, 3, 4, 5, 6, 7, 8, 9, 10, 11, 12, 13, 14, 15, 16, 17, 18, 19, or 20); and p is from 1 to 6 (i.e., 1, 2, 3, 4, 5, or 6).

In certain embodiments, L₁, L₂, L₃, and L₄ independently comprise or together comprise the structure selected from among:

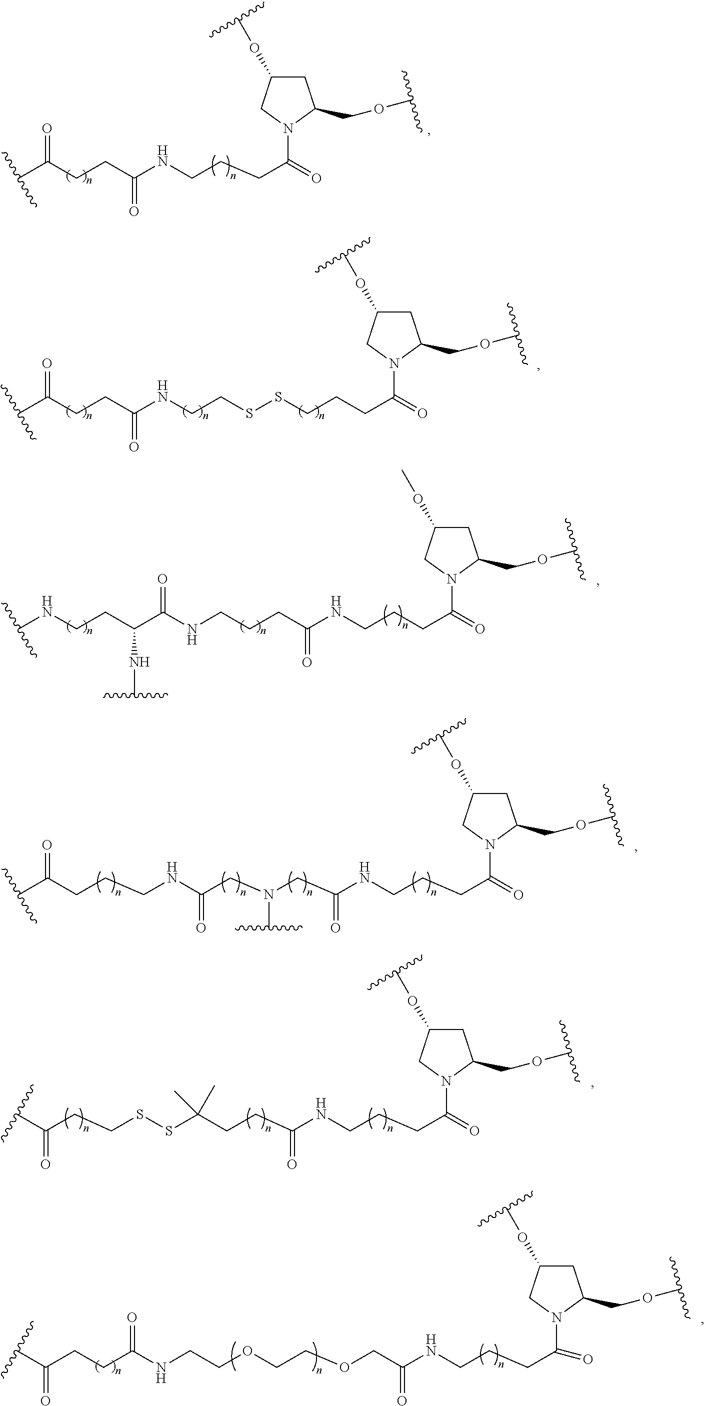

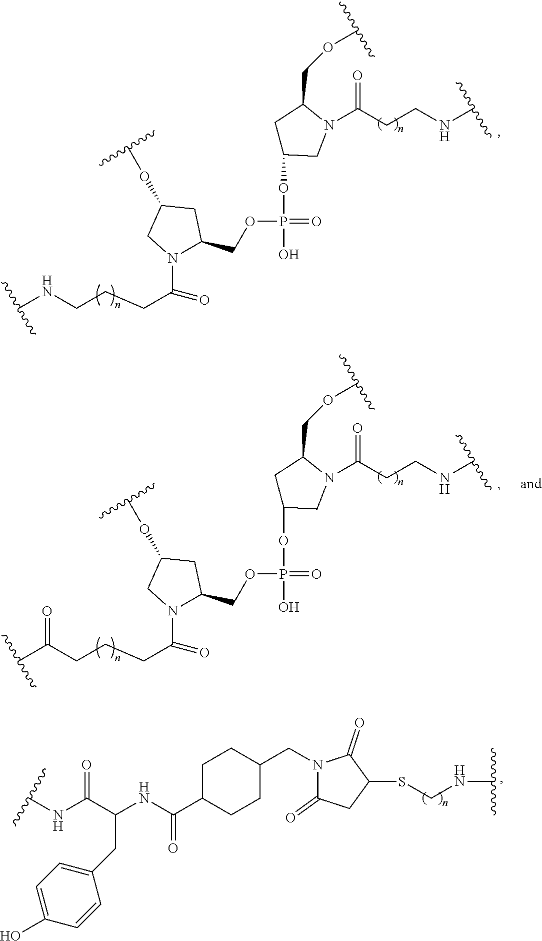

wherein each n is, independently, from 1 to 20.

In certain embodiments, L₁, L₂, L₃, and L₄ independently comprise or together comprise the structure selected from among:

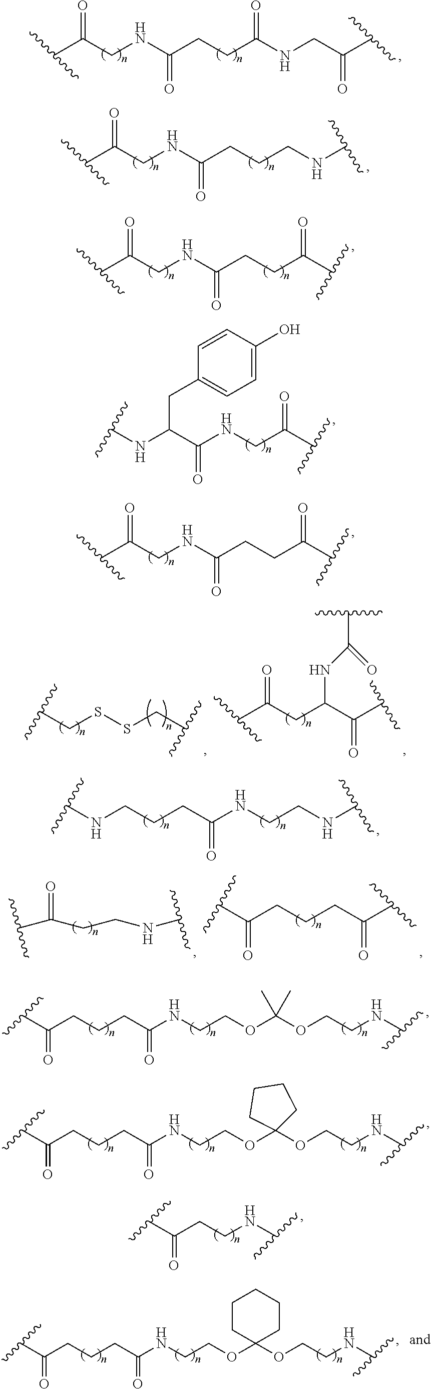

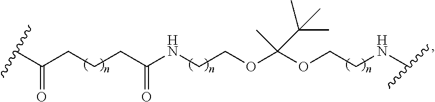

wherein each n is, independently, from 1 to 20.

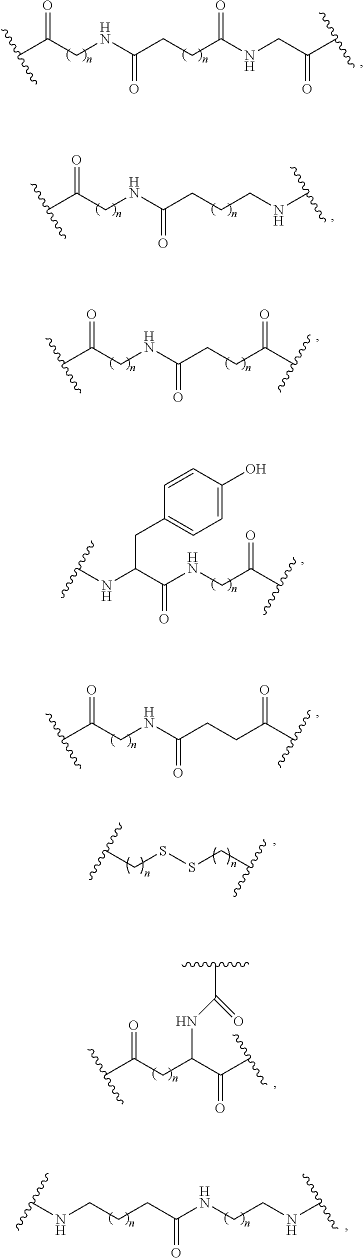

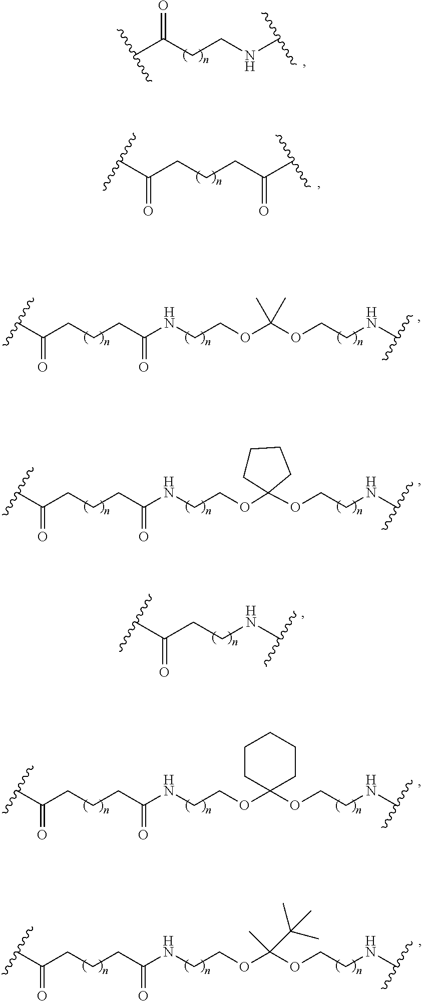

wherein each n is, independently, from 1 to 20.

In certain embodiments, L₁, L₂, L₃, and L₄ independently comprise or together comprise the structure selected from among:

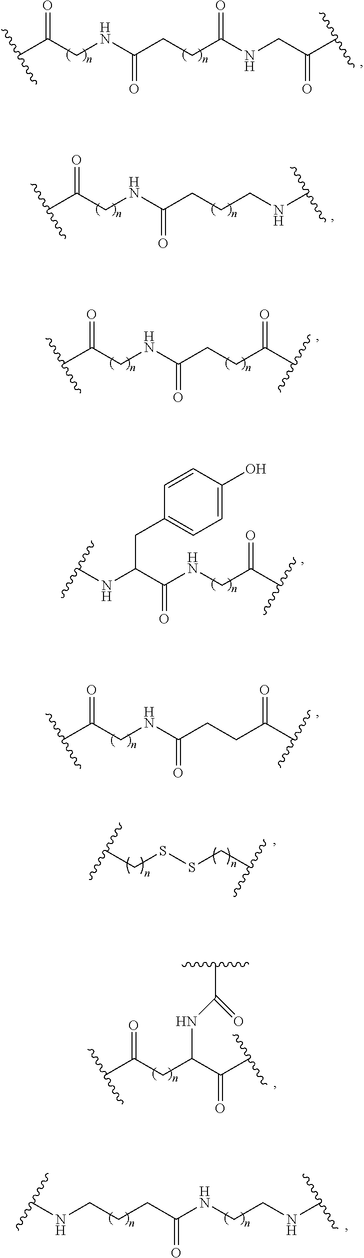

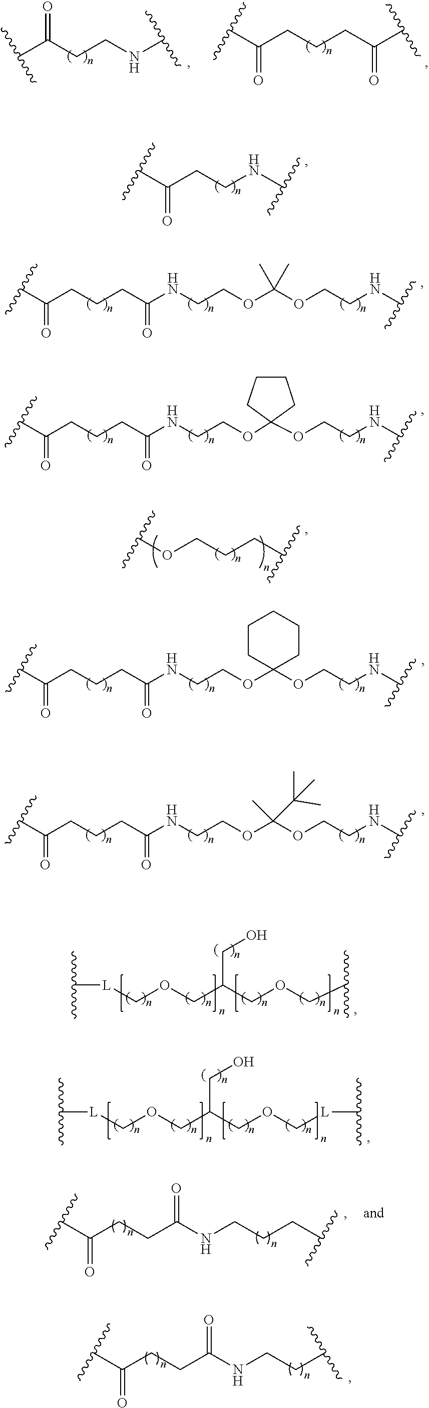

wherein each L is, independently, a phosphorous linking group; and each n is, independently, from 1 to 20.

In certain embodiments, L₁, L₂, L₃, and L₄ independently comprise or together comprise the structure selected from among:

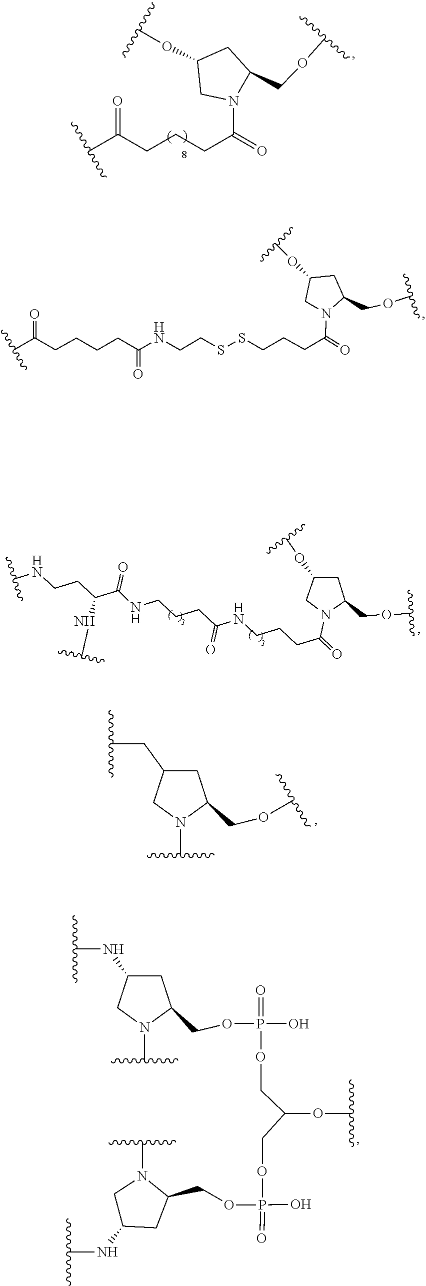 [SMILES: *OC[C@@H]1C[C@@H](O*)CN1C(=O)CCCC(*)=O ; *OC[C@@H]1C[C@@H](O*)CN1C(=O)CCCSSCCNC(=O)CCCCC(*)=O ; *NCC[C@@H](N*)C(=O)NCCCC(=O)NCCCC(=O)N1C[C@H](O*)C[C@H]1CO* ; *CC1C[C@@H](CO*)N(*)C1 ; *N[C@@H]1C[C@@H](COP(=O)(O)OCC(COP(=O)(O)OC[C@H]2C[C@H](N*)CN2*)O*)N(*)C1]

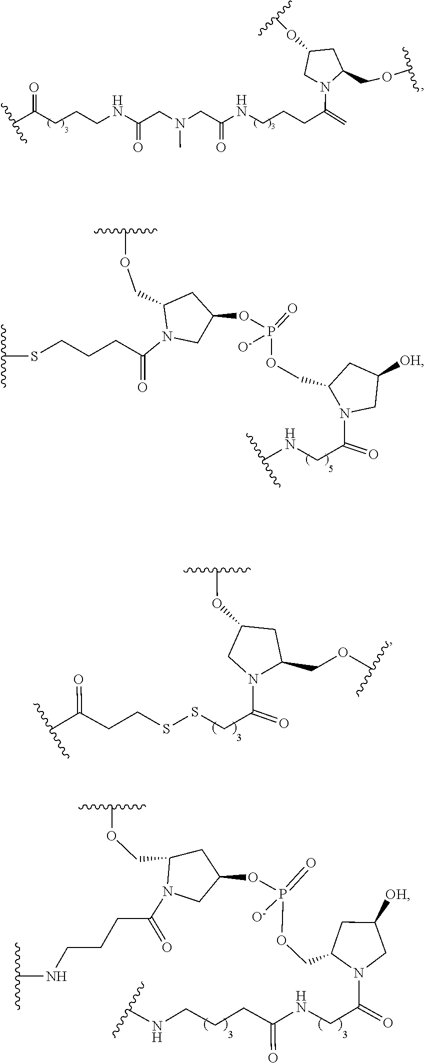 [SMILES: *OC[C@@H]1C[C@@H](O*)CN1C(=C)CCCNC(=O)CN(C)CC(=O)NCCCC(*)=O ; *NCC(=O)N1C[C@H](O)C[C@H]1COP(=O)([O-])O[C@@H]1C[C@@H](CO*)N(C(=O)CCCS*)C1 ; *OC[C@@H]1C[C@@H](O*)CN1C(=O)CSSCCC(*)=O ; *NCCCC(=O)NCC(=O)N1C[C@H](O)C[C@H]1COP(=O)([O-])O[C@@H]1C[C@@H](CO*)N(C(=O)CCCN*)C1]

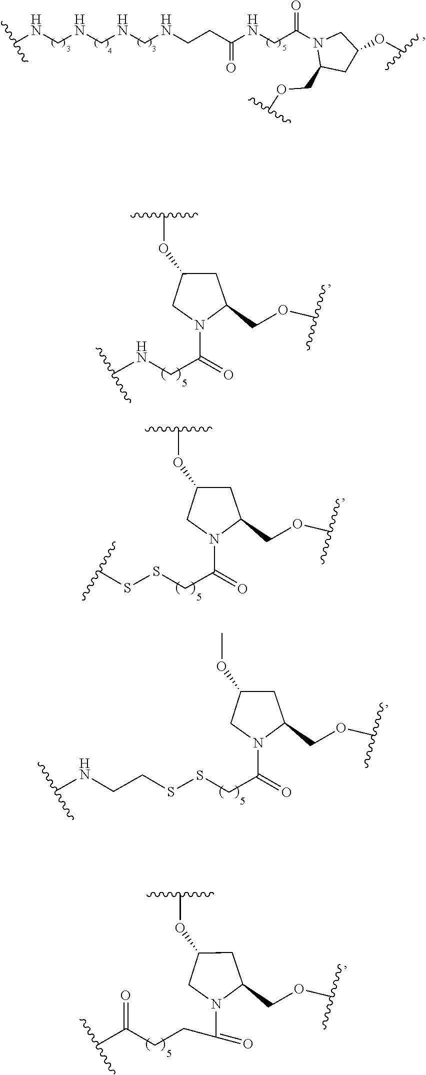 [SMILES: *NCNCNCNCCC(=O)NCC(=O)N1C[C@H](O*)C[C@H]1CO* ; *NCC(=O)N1C[C@H](O*)C[C@H]1CO* ; *OC[C@@H]1C[C@@H](O*)CN1C(=O)CSS* ; *NCCSSCC(=O)N1C[C@H](OC)C[C@H]1CO* ; *OC[C@@H]1C[C@@H](O*)CN1C(=O)CCC(*)=O]

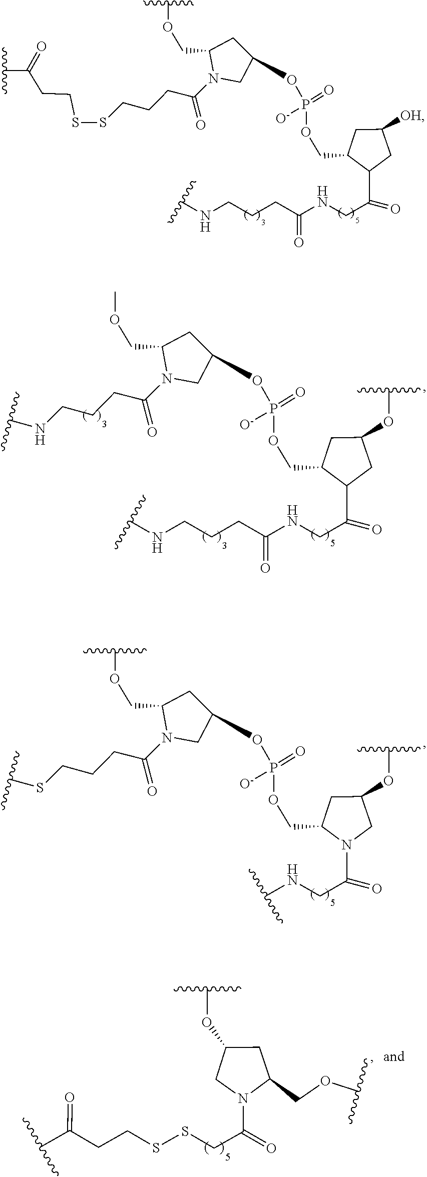 [SMILES: *NCCCC(=O)NCC(=O)C1C[C@H](O)C[C@H]1COP(=O)([O-])O[C@@H]1C[C@@H](CO*)N(C(=O)CCCSSCCC(*)=O)C1 ; *NCCCC(=O)NCC(=O)C1C[C@H](O*)C[C@H]1COP(=O)([O-])O[C@@H]1C[C@@H](COC)N(C(=O)CCCN*)C1 ; *NCC(=O)N1C[C@H](O*)C[C@H]1COP(=O)([O-])O[C@@H]1C[C@@H](CO*)N(C(=O)CCCS*)C1 ; *OC[C@@H]1C[C@@H](O*)CN1C(=O)CSSCCC(*)=O]

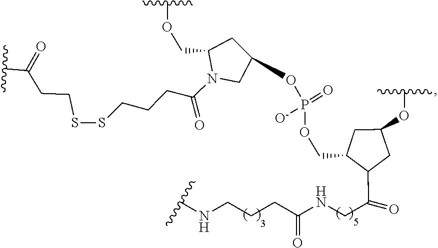 [SMILES: *NCCCC(=O)NCC(=O)C1C[C@H](O*)C[C@H]1COP(=O)([O-])O[C@@H]1C[C@@H](CO*)N(C(=O)CCCSSCCC(*)=O)C1]

In certain embodiments, L₁, L₂, L₃, and L₄ independently comprise or together comprise the structure selected from among:

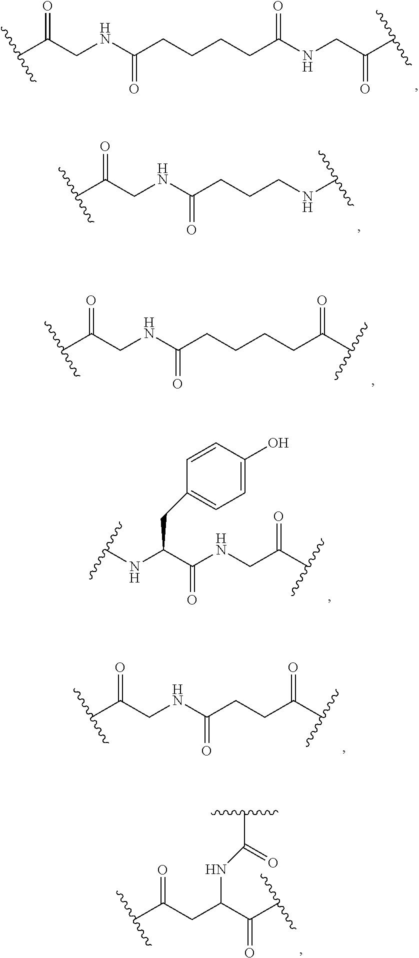 [SMILES: *C(=O)CNC(=O)CCCCC(=O)NCC(*)=O ; *NCCCC(=O)NCC(*)=O ; *C(=O)CCCCC(=O)NCC(*)=O ; *N[C@@H](Cc1ccc(O)cc1)C(=O)NCC(*)=O ; *C(=O)CCC(=O)NCC(*)=O ; *C(=O)CC(NC(*)=O)C(*)=O]

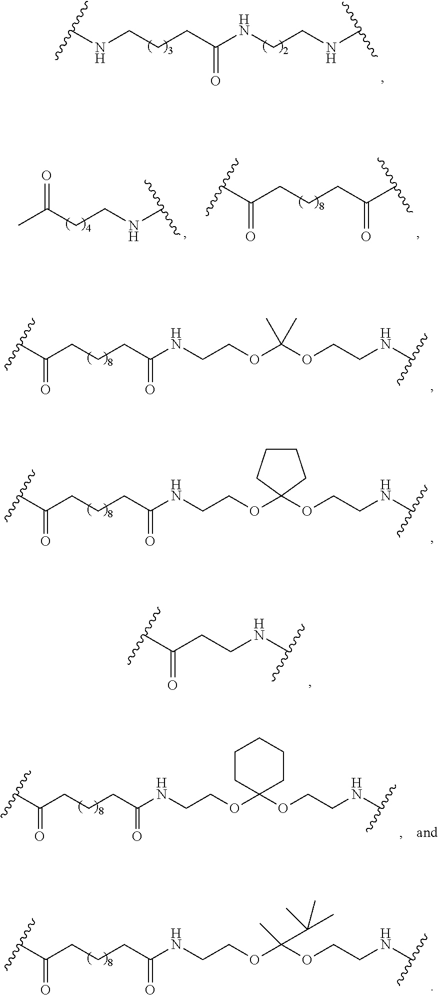

In certain embodiments, L₁, L₂, L₃, and L₄ independently comprise or together comprise the structure selected from among:

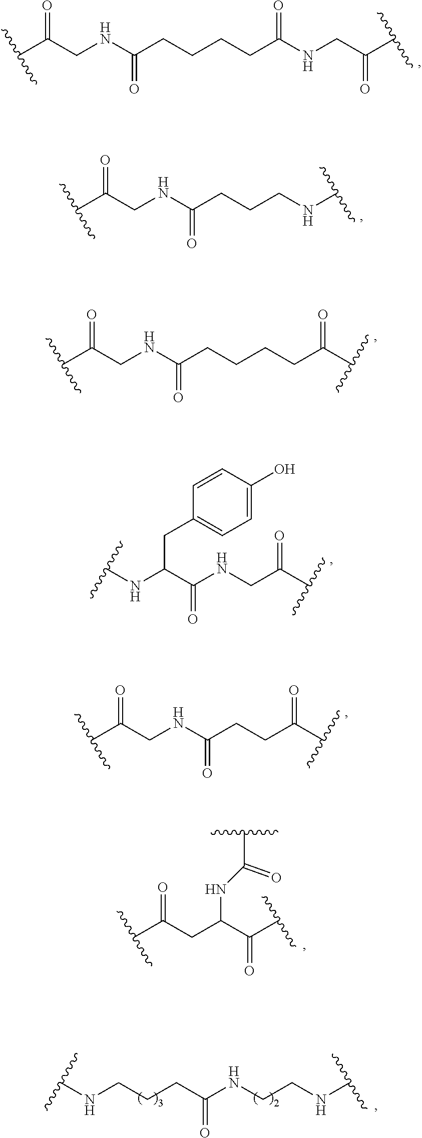

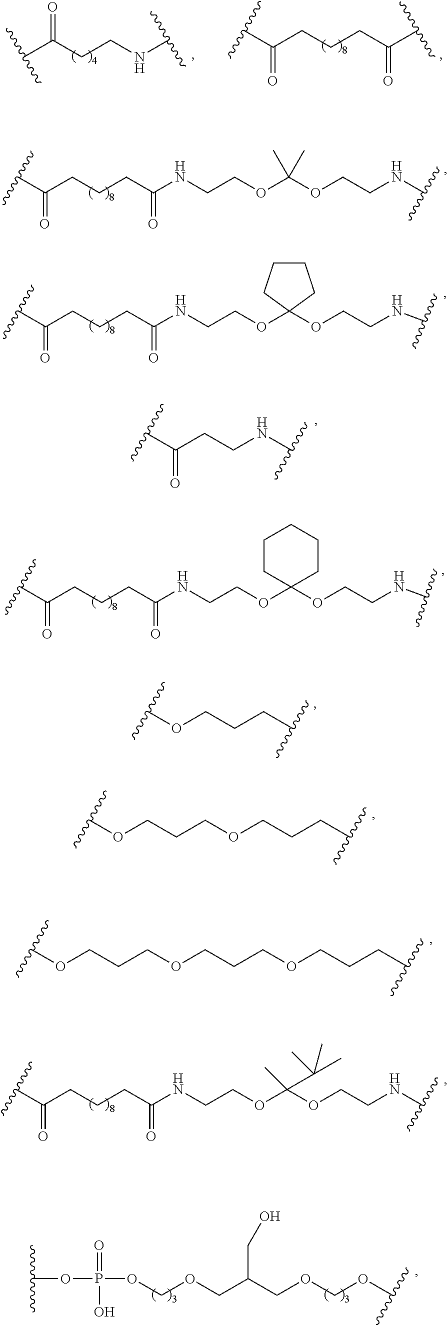

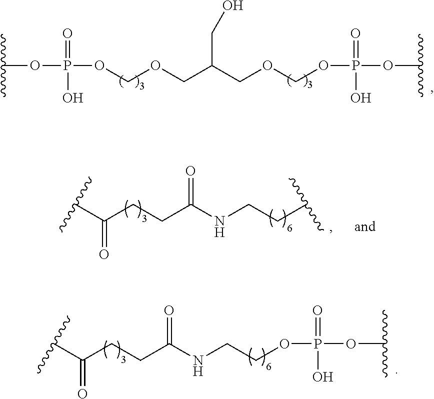

In certain embodiments, L₁, L₂, L₃, and L₄ independently comprise or together comprise the structure selected from among:

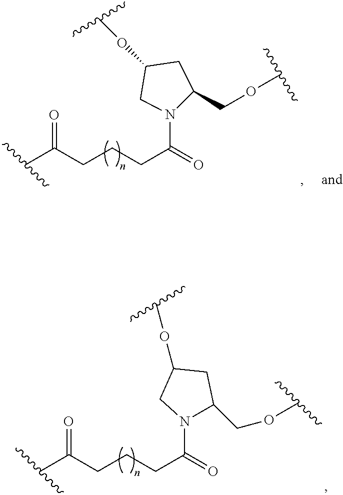

wherein n is from 1 to 20 (i.e., 1, 2, 3, 4, 5, 6, 7, 8, 9, 10, 11, 12, 13, 14, 15, 16, 17, 18, 19, or 20).

In certain embodiments, L₁, L₂, L₃, and L₄ independently comprise or together comprise the structure selected from among:

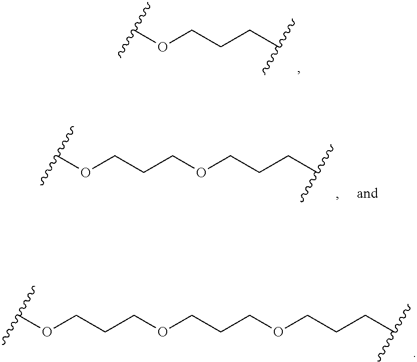 [SMILES: *CCCO* ; *CCCOCCCO* ; *CCCOCCCOCCCO*]

In certain embodiments, L₁, L₂, L₃, and L₄ independently comprise or together comprise the structure selected from among:

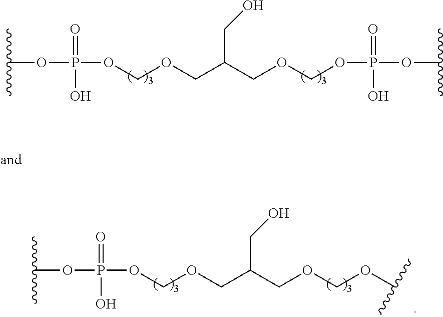

In certain embodiments, L₁, L₂, L₃, and L₄ independently comprise or together comprise the structure selected from among:

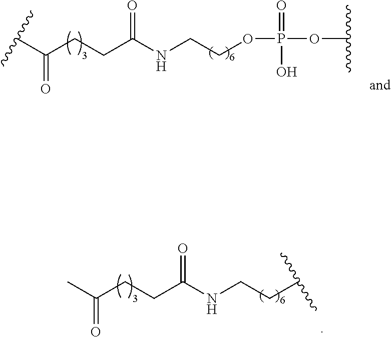

In certain embodiments, L₁, L₂, L₃, and L₄ independently comprise or together have the structure:

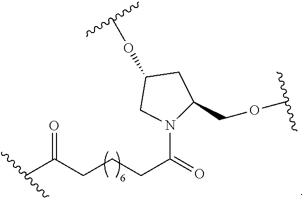

In certain embodiments, L₁, L₂, L₃, and L₄ independently comprise or together have the structure:

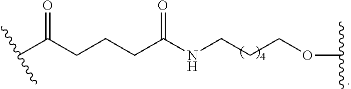

In certain embodiments, L₁, L₂, L₃, and L₄ independently comprise or together comprise the structure selected from among:

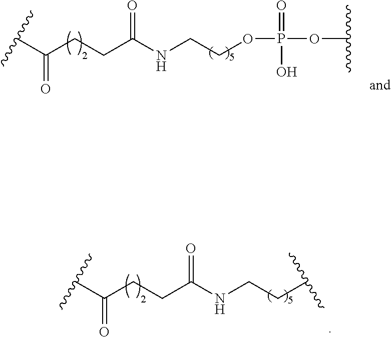

In certain embodiments, L₁, L₂, L₃, and L₄ independently comprise or together comprise the structure selected from among:

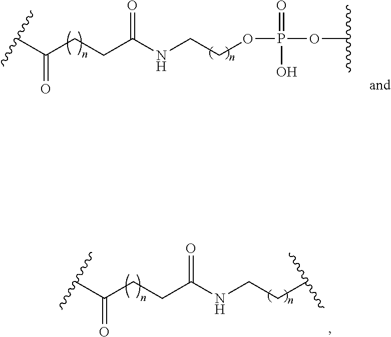

wherein each n is independently 0, 1, 2, 3, 4, 5, 6, or 7.

In some embodiments, each of L₁, L₂, L₃, and L₄ is independently absent, a bond, an optionally substituted alkyl linker, an optionally substituted polyethylene glycol (PEG) linker, an optionally substituted heteroalkyl linker (e.g., a C₁₋₂₀ alkyl linker in which any one or more carbon atoms is replaced with a heteroatom, such as N, O, P, or S), or an optionally substituted heteroaryl linker. In some embodiments, L₁ is an optionally substituted heteroaryl linker. In some embodiments, L₁ is an optionally substituted unsaturated heteroaryl linker. In certain embodiments, L₁ comprises the structure

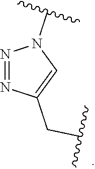 [SMILES: *Cc1cn(*)nn1]

In some embodiments, L₁ is an optionally substituted heteroalkyl linker. In certain embodiments, L₁ comprises the structure

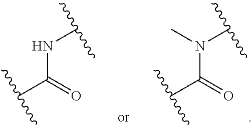

In certain embodiments, L₁ comprises the structure

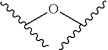 [SMILES: *O*]

### or —N(CH₃)—.

In some embodiments, L₂ is an optionally substituted PEG linker. In some embodiments, the PEG linker is five PEG units in length. In some embodiments, the PEG linker is four PEG units in length. In some embodiments, the PEG linker is three PEG units in length. In some embodiments, L₂ is an optionally substituted alkyl linker. In some embodiments, L₂ is an optionally substituted C₁₋₂₀ alkyl linker. In certain embodiments, L₂ is an optionally substituted C₈ alkyl linker.

In some embodiments, L₃ is an optionally substituted heteroaryl linker. In some embodiments, L₃ is an optionally substituted partially unsaturated heteroaryl linker. In certain embodiments, L₃ comprises the structure

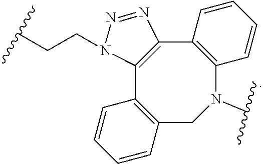 [SMILES: *CCn1nnc2c1-c1ccccc1CN(*)c1ccccc1-2]

In some embodiments, L₄ is an optionally substituted heteroalkyl linker. In some embodiments, the heteroalkyl linker is substituted with one or more ═O substituents. In some embodiments, the heteroalkyl linker comprises two substituents joined together to form an optionally substituted carbocyclyl ring. In certain embodiments, L₄ comprises the structure

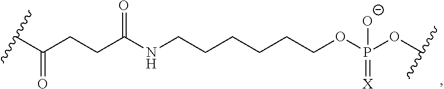

wherein X is O or S. In certain embodiments, L₄ comprises the structure

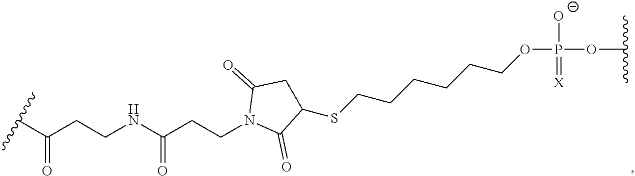

wherein X is O or S.

In some embodiments, L₁, L₂, L₃, and L₄ together comprise the structure

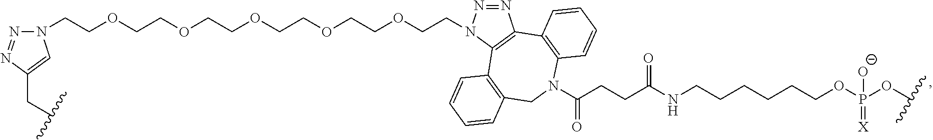

wherein X is O or S. In some embodiments, L₁, L₂, L₃, and L₄ together comprise the structure

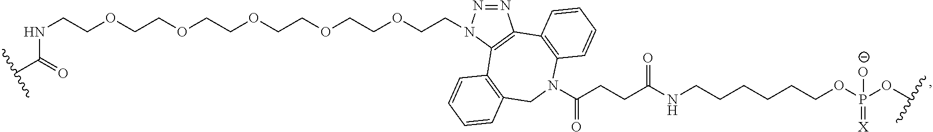

wherein X is O or S. In some embodiments, L₁, L₂, L₃, and L₄ together comprise the structure

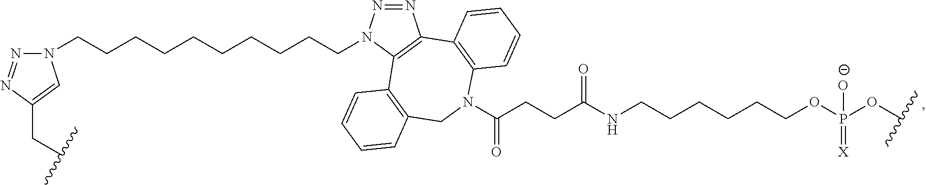

wherein X is O or S. In some embodiments, L₁, L₂, L₃, and L₄ together comprise the structure

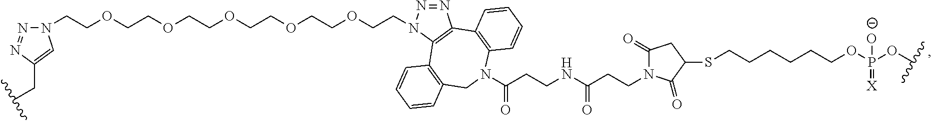

wherein X is O or S. In some embodiments, L₁, L₂, L₃, and L₄ together comprise the structure

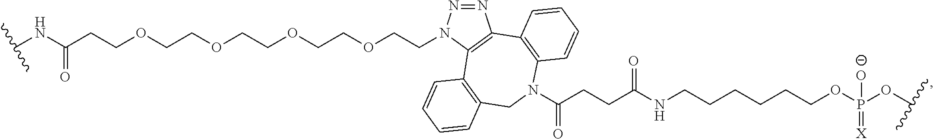

wherein X is O or S. In some embodiments, L₁, L₂, L₃, and L₄ together comprise the structure

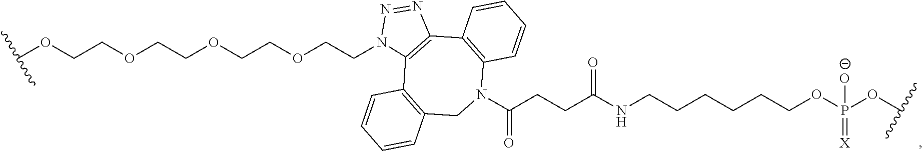

wherein X is O or S.

### Methods of Making Compounds

In some aspects, the disclosure relates to methods of making the compounds and compositions comprising TrkB ligands as disclosed herein.

Compounds of the present disclosure can be made by means known in the art of organic synthesis. Methods for optimizing reaction conditions, and minimizing competing by-products, if necessary, are known in the art. Reaction optimization and scale-up may advantageously utilize high-speed parallel synthesis equipment and computer-controlled microreactors (e.g., Design and Optimization in Organic Synthesis, 2^{nd} Edition, Carlson R, Ed, 2005; Elsevier Science Ltd.; Jähnisch, K et al., Angew. Chem. Int. Ed. Engl. 2004 43:406; and references therein). Additional reaction schemes and protocols may be determined by the skilled artisan by use of commercially available structure-searchable database software, for instance, SciFinder® (CAS division of the American Chemical Society) and CrossFire Beilstein® (Elsevier MDL), or by appropriate keyword searching using an internet search engine such as Google® or keyword databases such as the U.S. Patent and Trademark Office text database.

As can be appreciated by the skilled artisan, methods of synthesizing the compounds of the formulae herein will be evident to those of ordinary skill in the art, including in the schemes and examples herein. Additionally, the various synthetic steps may be performed in an alternate sequence or order to give the desired compounds. In addition, the solvents, temperatures, reaction durations, etc. delineated herein are for purposes of illustration only and one of ordinary skill in the art will recognize that variation of the reaction conditions can produce the desired compounds of the present disclosure.

The compounds herein may also contain linkages (e.g., carbon-carbon bonds) wherein bond rotation is restricted about that particular linkage, e.g., restriction resulting from the presence of a ring or double bond. Accordingly, all cis/trans and E/Z isomers are expressly included in the present disclosure. The compounds herein may also be represented in multiple tautomeric forms; in such instances, the present disclosure expressly includes all tautomeric forms of the compounds described herein, even though only a single tautomeric form may be represented. All such isomeric forms of such compounds herein are expressly included in the present disclosure. All crystal forms and polymorphs of the compounds described herein are expressly included in the present disclosure. Also embodied are extracts and fractions comprising compounds of the present disclosure. The term “isomers” is intended to include diastereoisomers, enantiomers, regioisomers, structural isomers, rotational isomers, tautomers, and the like. For compounds which contain one or more stereogenic centers, e.g., chiral compounds, the methods of the present disclosure may be carried out with an enantiomerically enriched compound, a racemate, or a mixture of diastereomers. All isomers of compounds delineated herein are expressly included in the present disclosure.

Preferred enantiomerically enriched compounds have an enantiomeric excess of 50% or more. More preferably, the compound has an enantiomeric excess of 60%, 70%, 80%, 90%, 95%, 98%, 99%, or more. In preferred embodiments, only one enantiomer or diastereomer of a chiral compound of the present disclosure is administered to cells or a subject.

### Methods of Treatment

In one aspect, provided are methods of treating a subject suffering from or susceptible to a disorder or disease, comprising administering to the subject an effective amount of a compound or pharmaceutical composition described herein.

In other aspects, provided are methods of treating a subject suffering from or susceptible to a disorder or disease, wherein the subject has been identified as in need of modulation of the function of a protein, comprising administering to said subject in need thereof, an effective amount of a compound or pharmaceutical composition described herein, such that said subject is treated for said disorder.

In one aspect, provided are methods of delivering a therapeutic oligonucleotide to the brain of a subject, comprising contacting the subject with a compound or pharmaceutical composition described herein, in an amount and under conditions sufficient to target the brain. In some embodiments, the therapeutic oligonucleotide is delivered to one or more brain regions selected from the group consisting of the striatum, the cerebellum, the brain stem, the hippocampus, the frontal cortex, and the spinal cord.

In certain embodiments, provided are methods of treating a disease, disorder or symptom thereof, wherein the disease is a central nervous system (CNS) disease, disorder, or symptom thereof. In some embodiments, the disease is a neurodegenerative disease, disorder, or symptom thereof. In some embodiments, the disease is Alzheimer's disease, or a symptom thereof.

Exemplary CNS disorders include, but are not limited to, neurotoxicity and/or neurotrauma, stroke, multiple sclerosis, spinal cord injury, epilepsy, a mental disorder, a sleep condition, a movement disorder, nausea and/or emesis, amyotrophic lateral sclerosis, Alzheimer's disease, and drug addiction.

In certain embodiments, the CNS disorder is neurotoxicity and/or neurotrauma, e.g., for example, as a result of acute neuronal injury (e.g., traumatic brain injury (TBI), stroke, epilepsy) or a chronic neurodegenerative disorder (e.g., multiple sclerosis, Parkinson's disease, Huntington's disease, amyotrophic lateral sclerosis, Alzheimer's disease). In certain embodiments, the compounds of the present disclosure provide a neuroprotective effect, e.g., against an acute neuronal injury or a chronic neurodegenerative disorder.

In certain embodiments, the CNS disorder is stroke (e.g., ischemic stroke).

In certain embodiments, the CNS disorder is multiple sclerosis.

In certain embodiments, the CNS disorder is spinal cord injury.

In certain embodiments, the CNS disorder is epilepsy.

In certain embodiments, the CNS disorder is a mental disorder, e.g., for example, depression, anxiety or anxiety-related conditions, a learning disability, somatic symptom disorder, or schizophrenia or schizoaffective disorder.

In certain embodiments, the CNS disorder is depression. “Depression” includes, but is not limited to, depressive disorders or conditions, such as, for example, major depressive disorders (e.g., unipolar depression), treatment-resistant depression, dysthymic disorders (e.g., chronic, mild depression), bipolar disorders (e.g., manic depression), seasonal affective disorder, and/or depression associated with substance abuse or substance abuse disorder (e.g., withdrawal). The depression can be clinical or subclinical depression. The depression can be associated with or premenstrual syndrome and/or premenstrual dysphoric disorder.

In certain embodiments, the CNS disorder is anxiety. “Anxiety” includes, but is not limited to, anxiety and anxiety-related conditions, such as, for example, clinical anxiety, panic disorder, agoraphobia, generalized anxiety disorder (GAD), specific phobia, social phobia, obsessive-compulsive disorder, acute stress disorder, post-traumatic stress disorder, adjustment disorders with anxious features, anxiety disorder associated with depression, anxiety disorder due to general medical conditions, and substance-induced anxiety disorders, anxiety associated with substance abuse or substance use disorder (e.g., withdrawal, dependence, reinstatement) and anxiety associated with nausea and/or emesis. This treatment may also be to induce or promote sleep in a subject (e.g., for example, a subject with anxiety).

In certain embodiments, the CNS disorder is a learning disorder (e.g., attention deficit disorder (ADD)).

In certain embodiments, the CNS disorder is schizophrenia or schizoaffective disorder.

In certain embodiments, the CNS disorder is a sleep condition. “Sleep conditions” include, but are not limited to, insomnia, narcolepsy, sleep apnea, restless legs syndrome (RLS), delayed sleep phase syndrome (DSPS), periodic limb movement disorder (PLMD), hypopnea syndrome, rapid eye movement behavior disorder (RBD), shift work sleep condition (SWSD), and sleep problems (e.g., parasomnias) such as nightmares, night terrors, sleep talking, head banging, snoring, and clenched jaw and/or grinding of teeth (bruxism).

In certain embodiments, the CNS disorder is a movement disorder, e.g., basal ganglia disorders, such as, for example, Parkinson's disease, levodopa-induced dyskinesia, Huntington's disease, Gilles de la Tourette's syndrome, tardive dyskinesia, and dystonia.

In certain embodiments, the CNS disorder is Alzheimer's disease.

In certain embodiments, the CNS disorder is amyotrophic lateral sclerosis (ALS).

In certain embodiments, the CNS disorder is nausea and/or emesis.

In certain embodiments, the CNS disorder is drug addiction (e.g., for instance, addiction to opiates, nicotine, cocaine, psychostimulants, or alcohol).

The term “neurological disease” (including, e.g., “neurodegenerative diseases”) refers to any disease of the nervous system, including diseases that involve the central nervous system (brain, brainstem, and cerebellum), the peripheral nervous system (including cranial nerves), and the autonomic nervous system (parts of which are located in both central and peripheral nervous system). Neurodegenerative diseases refer to a type of neurological disease marked by the loss of nerve cells, including, but not limited to, Alzheimer's disease, Parkinson's disease, amyotrophic lateral sclerosis, tauopathies (including frontotemporal dementia), and Huntington's disease. Examples of neurological diseases include, but are not limited to, headache, stupor and coma, dementia, seizure, sleep disorders, trauma, infections, neoplasms, neuro-ophthalmology, movement disorders, demyelinating diseases, spinal cord disorders, and disorders of peripheral nerves, muscle, and neuromuscular junctions. Substance abuse or substance use disorder (SUD) and mental illness, including, but not limited to, bipolar disorder, schizophrenia, and schizoaffective disorder, are also included in the definition of neurological diseases. Further examples of neurological diseases include acquired epileptiform aphasia; acute disseminated encephalomyelitis; adrenoleukodystrophy; agenesis of the corpus callosum; agnosia; Aicardi syndrome; Alexander disease; Alpers' disease; alternating hemiplegia; Alzheimer's disease; amyotrophic lateral sclerosis; anencephaly; Angelman syndrome; angiomatosis; anoxia; aphasia; apraxia; arachnoid cysts; arachnoiditis; Arnold-Chiari malformation; arteriovenous malformation; Asperger syndrome; ataxia telangiectasia; attention deficit hyperactivity disorder; autism; autonomic dysfunction; back pain; Batten disease; Behcet's disease; Bell's palsy; benign essential blepharospasm; benign focal; amyotrophy; benign intracranial hypertension; Binswanger's disease; blepharospasm; Bloch Sulzberger syndrome; brachial plexus injury; brain abscess; brain injury; brain tumors (including glioblastoma multiforme); spinal tumor; Brown-Sequard syndrome; Canavan disease; carpal tunnel syndrome (CTS); causalgia; central pain syndrome; central pontine myelinolysis; cephalic disorder; cerebral aneurysm; cerebral arteriosclerosis; cerebral atrophy; cerebral gigantism; cerebral palsy; Charcot-Marie-Tooth disease; chemotherapy-induced neuropathy and neuropathic pain; Chiari malformation; chorea; chronic inflammatory demyelinating polyneuropathy (CIDP); chronic pain; chronic regional pain syndrome; Coffin Lowry syndrome; coma, including persistent vegetative state; congenital facial diplegia; corticobasal degeneration; cranial arteritis; craniosynostosis; Creutzfeldt-Jakob disease; cumulative trauma disorders; Cushing's syndrome; cytomegalic inclusion body disease (CIBD); cytomegalovirus infection; dancing eyes-dancing feet syndrome; Dandy-Walker syndrome; Dawson disease; De Morsier's syndrome; Dejerine-Klumpke palsy; dementia; dermatomyositis; diabetic neuropathy; diffuse sclerosis; dysautonomia; dysgraphia; dyslexia; dystonias; early infantile epileptic encephalopathy; empty sella syndrome; encephalitis; encephaloceles; encephalotrigeminal angiomatosis; epilepsy; Erb's palsy; essential tremor; Fabry's disease; Fahr's syndrome; fainting; familial spastic paralysis; febrile seizures; Fisher syndrome; Friedreich's ataxia; frontotemporal dementia and other “tauopathies”; Gaucher's disease; Gerstmann's syndrome; giant cell arteritis; giant cell inclusion disease; globoid cell leukodystrophy; Guillain-Barre syndrome; HTLV-1 associated myelopathy; Hallervorden-Spatz disease; head injury; headache; hemifacial spasm; hereditary spastic paraplegia; heredopathia atactica polyneuritiformis; herpes zoster oticus; herpes zoster; Hirayama syndrome; HIV-associated dementia and neuropathy (see also neurological manifestations of AIDS); holoprosencephaly; Huntington's disease and other polyglutamine repeat diseases; hydranencephaly; hydrocephalus; hypercortisolism; hypoxia; immune-mediated encephalomyelitis; inclusion body myositis; incontinentia pigmenti; infantile; phytanic acid storage disease; Infantile Refsum disease; infantile spasms; inflammatory myopathy; intracranial cyst; intracranial hypertension; Joubert syndrome; Kearns-Sayre syndrome; Kennedy disease; Kinsbourne syndrome; Klippel Feil syndrome; Krabbe disease; Kugelberg-Welander disease; kuru; Lafora disease; Lambert-Eaton myasthenic syndrome; Landau-Kleffner syndrome; lateral medullary (Wallenberg) syndrome; learning disabilities; Leigh's disease; Lennox-Gastaut syndrome; Lesch-Nyhan syndrome; leukodystrophy; Lewy body dementia; lissencephaly; locked-in syndrome; Lou Gehrig's disease (also known as motor neuron disease or amyotrophic lateral sclerosis); lumbar disc disease; lyme disease-neurological sequelae; Machado-Joseph disease; macrencephaly; megalencephaly; Melkersson-Rosenthal syndrome; Menieres disease; meningitis; Menkes disease; metachromatic leukodystrophy; microcephaly; migraine; Miller Fisher syndrome; mini-strokes; mitochondrial myopathies; Mobius syndrome; monomelic amyotrophy; motor neurone disease; moyamoya disease; mucopolysaccharidoses; multi-infarct dementia; multifocal motor neuropathy; multiple sclerosis and other demyelinating disorders; multiple system atrophy with postural hypotension; muscular dystrophy; myasthenia gravis; myelinoclastic diffuse sclerosis; myoclonic encephalopathy of infants; myoclonus; myopathy; myotonia congenital; narcolepsy; neurofibromatosis; neuroleptic malignant syndrome; neurological manifestations of AIDS; neurological sequelae of lupus; neuromyotonia; neuronal ceroid lipofuscinosis; neuronal migration disorders; Niemann-Pick disease; O'Sullivan-McLeod syndrome; occipital neuralgia; occult spinal dysraphism sequence; Ohtahara syndrome; olivopontocerebellar atrophy; opsoclonus myoclonus; optic neuritis; orthostatic hypotension; overuse syndrome; paresthesia; Parkinson's disease; paramyotonia congenita; paraneoplastic diseases; paroxysmal attacks; Parry Romberg syndrome; Pelizaeus-Merzbacher disease; periodic paralyses; peripheral neuropathy; painful neuropathy and neuropathic pain; persistent vegetative state; pervasive developmental disorders; photic sneeze reflex; phytanic acid storage disease; Pick's disease; pinched nerve; pituitary tumors; polymyositis; porencephaly; Post-Polio syndrome; postherpetic neuralgia (PHN); postinfectious encephalomyelitis; postural hypotension; Prader-Willi syndrome; primary lateral sclerosis; prion diseases; progressive; hemifacial atrophy; progressive multifocal leukoencephalopathy; progressive sclerosing poliodystrophy; progressive supranuclear palsy; pseudotumor cerebri; Ramsay-Hunt syndrome (Type I and Type II); Rasmussen's Encephalitis; reflex sympathetic dystrophy syndrome; Refsum disease; repetitive motion disorders; repetitive stress injuries; restless legs syndrome; retrovirus-associated myelopathy; Rett syndrome; Reye's syndrome; Saint Vitus Dance; Sandhoff disease; Schilder's disease; schizencephaly; septo-optic dysplasia; shaken baby syndrome; shingles; Shy-Drager syndrome; Sjogren's syndrome; sleep apnea; Soto's syndrome; spasticity; spina bifida; spinal cord injury; spinal cord tumors; spinal muscular atrophy; stiff-person syndrome; stroke; Sturge-Weber syndrome; subacute sclerosing panencephalitis; subarachnoid hemorrhage; subcortical arteriosclerotic encephalopathy; sydenham chorea; syncope; syringomyelia; tardive dyskinesia; Tay-Sachs disease; temporal arteritis; tethered spinal cord syndrome; Thomsen disease; thoracic outlet syndrome; tic douloureux; Todd's paralysis; Tourette syndrome; transient ischemic attack; transmissible spongiform encephalopathies; transverse myelitis; traumatic brain injury; tremor; trigeminal neuralgia; tropical spastic paraparesis; tuberous sclerosis; vascular dementia (multi-infarct dementia); vasculitis including temporal arteritis; Von Hippel-Lindau Disease (VHL); Wallenberg's syndrome; Werdnig-Hoffman disease; West syndrome; whiplash; Williams syndrome; Wilson's disease; and Zellweger syndrome.

In certain embodiments, the subject is a mammal, preferably a primate or a human.

In another embodiment, provided are methods as described above, wherein the effective amount of the compounds provided herein is as described above.

In another embodiment, provided are methods as described above, wherein the compounds provided herein is administered intrathecally, intravenously, intramuscularly, subcutaneously, intracerebroventricularly, orally, or topically. In certain embodiments, the compound is administered intrathecally.

In other embodiments, provided are methods as described above, wherein the compound of any of the formulae provided herein is administered alone or in combination with one or more other therapeutics. In a further embodiment, the additional therapeutic agent is a central nervous system (CNS) disease agent.

Another object of the present disclosure is the use of a compound as described herein in the manufacture of a medicament for use in the treatment of a disorder or disease. Another object of the present disclosure is the use of a compound as described herein for use in the treatment of a disorder or disease.

### Pharmaceutical Compositions

In one aspect, provided are pharmaceutical compositions comprising any of the compounds described herein and a pharmaceutically acceptable carrier or pharmaceutically acceptable excipient.

A compound or composition, as described herein, can be administered in combination with one or more additional therapeutic agents (e.g., therapeutically and/or prophylactically active agents). The compounds or compositions can be administered in combination with additional therapeutic agents that improve their activity (e.g., activity (e.g., potency and/or efficacy) in treating a disease in a subject in need thereof, in preventing a disease in a subject in need thereof, and/or in reducing the risk to develop a disease in a subject in need thereof), improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described herein including a compound described herein and an additional therapeutic agent exhibits a synergistic effect that is absent in a pharmaceutical composition including one of the compounds described herein or the additional therapeutic agent, but not both.

The compound or composition can be administered concurrently with, prior to, or subsequent to one or more additional therapeutic agents, which may be useful as, e.g., combination therapies. Therapeutic agents include therapeutically active agents. Therapeutic agents also include prophylactically active agents. Therapeutic agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNA, RNAs, nucleotides, nucleosides, oligonucleotides, antisense oligonucleotides, lipids, hormones, vitamins, and cells. In certain embodiments, the additional therapeutic agent is a therapeutic agent useful for treating and/or preventing a disease (e.g., CNS disorder). Each additional therapeutic agent may be administered at a dose and/or on a time schedule determined for that therapeutic agent. The additional therapeutic agents may also be administered together with each other and/or with the compound or composition described herein in a single dose or administered separately in different doses. The particular combination to employ in a regimen will take into account compatibility of the compound described herein with the additional therapeutic agent(s) and/or the desired therapeutic and/or prophylactic effect to be achieved. In general, it is expected that the additional therapeutic agent(s) in combination be utilized at levels that do not exceed the levels at which they are utilized individually. In some embodiments, the levels utilized in combination will be lower than those utilized individually.

In one aspect, provided are kits comprising an effective amount of a compound provided herein, in unit dosage form, together with instructions for administering the compound to a subject suffering from or susceptible to a disease or disorder.

The term “pharmaceutically acceptable salts” or “pharmaceutically acceptable carrier” is meant to include salts of the active compounds which are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydroiodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science 66:1-19 (1977)). Certain specific compounds of the present disclosure contain both basic and acidic functionalities that allow the compounds to be converted into either base or acid addition salts. Other pharmaceutically acceptable carriers known to those of skill in the art are suitable for the present disclosure.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present disclosure.

In addition to salt forms, the present disclosure provides compounds which are in a prodrug form. Prodrugs of the compounds described herein are those compounds that readily undergo chemical changes under physiological conditions to provide the compounds of the present disclosure. Additionally, prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme or chemical reagent.

Certain compounds of the present disclosure can exist in unsolvated forms as well as solvated forms, including hydrated forms. In general, the solvated forms are equivalent to unsolvated forms and are intended to be encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

The present disclosure also provides a pharmaceutical composition, comprising an effective amount of a compound described herein and a pharmaceutically acceptable excipient. In an embodiment, a compound of any of the formulae provided herein is administered to a subject using a pharmaceutically acceptable formulation, e.g., a pharmaceutically acceptable formulation that provides sustained delivery of the compound to a subject for at least 12 hours, 24 hours, 36 hours, 48 hours, one week, two weeks, three weeks, or four weeks after the pharmaceutically-acceptable formulation is administered to the subject.

Actual dosage levels and time course of administration of the active ingredients in the pharmaceutical compositions of the disclosure may be varied so as to obtain an amount of the active ingredient which is effective to achieve the desired therapeutic response for a particular subject, composition, and mode of administration, while being acceptably tolerant to the subject.

In use, at least one compound according to the present disclosure is administered in a pharmaceutically effective amount to a subject in need thereof in a pharmaceutical carrier by intravenous, intrathecal, intramuscular, subcutaneous, or intracerebroventricular injection (ICV) or by oral administration or topical application. In accordance with the present disclosure, a compound of the disclosure may be administered alone or in conjunction with a second, different therapeutic. By “in conjunction with” is meant together, substantially simultaneously, or sequentially. In one embodiment, a compound of the disclosure is administered acutely. The compound of the disclosure may therefore be administered for a short course of treatment, such as for about 1 day to about 1 week. In another embodiment, the compound of the disclosure may be administered over a longer period of time to ameliorate chronic disorders, such as, for example, for about one week to several months depending upon the condition to be treated.

By “pharmaceutically effective amount,” as used herein, is meant an amount of a compound of the disclosure, high enough to significantly positively modify the condition to be treated but low enough to avoid serious side effects (at a reasonable benefit/risk ratio), within the scope of sound medical judgment. A pharmaceutically effective amount of a compound of the disclosure will vary with the particular goal to be achieved, the age and physical condition of the patient being treated, the severity of the underlying disease, the duration of treatment, the nature of concurrent therapy and the specific compound employed. For example, a therapeutically effective amount of a compound of the disclosure administered to a child or a neonate will be reduced proportionately in accordance with sound medical judgment. The effective amount of a compound of the disclosure will thus be the minimum amount which will provide the desired effect.

A decided practical advantage of the present disclosure is that the compound may be administered in a convenient manner such as by intrathecal, intravenous, intramuscular, subcutaneous, oral, or intra-cerebroventricular injection routes or by topical application, such as in creams or gels. Depending on the route of administration, the active ingredients which comprise a compound of the disclosure may be required to be coated in a material to protect the compound from the action of enzymes, acids and other natural conditions which may inactivate the compound. In order to administer a compound of the disclosure by a mode other than parenteral administration, the compound can be coated by, or administered with, a material to prevent inactivation.

The compound may be administered parenterally or intraperitoneally. Dispersions can also be prepared, for example, in glycerol, liquid polyethylene glycols, and mixtures thereof, and in oils.

Some examples of substances which can serve as pharmaceutical excipients, or pharmaceutical carriers (which terms are used interchangeably herein), are sugars, such as lactose, glucose and sucrose; starches such as corn starch and potato starch; cellulose and its derivatives such as sodium carboxymethycellulose, ethylcellulose and cellulose acetates; powdered tragancanth; malt; gelatin; talc; stearic acids; magnesium stearate; calcium sulfate; vegetable oils, such as peanut oils, cotton seed oil, sesame oil, olive oil, corn oil, and oil of theobroma; polyols such as propylene glycol, glycerine, sorbitol, mannitol, and polyethylene glycol; agar; alginic acids; pyrogen-free water; isotonic saline; and phosphate buffer solution; skim milk powder; as well as other non-toxic compatible substances used in pharmaceutical formulations such as Vitamin C, estrogen and echinacea, for example. Wetting agents and lubricants such as sodium lauryl sulfate, as well as coloring agents, flavoring agents, lubricants, excipients, tableting agents, stabilizers, antioxidants, and preservatives, can also be present. Solubilizing agents, including for example, cremaphore, and beta-cyclodextrins, can also be used in the pharmaceutical compositions herein.

Pharmaceutical compositions comprising the active compounds of the present disclosure (or prodrugs thereof) can be manufactured by means of conventional mixing, dissolving, granulating, dragee-making levigating, emulsifying, encapsulating, entrapping, or lyophilization processes. The compositions can be formulated in conventional manner using one or more physiologically acceptable carriers, diluents, excipients, or auxiliaries, which facilitate processing of the active compounds into preparations that can be used pharmaceutically. The compositions herein can be made by combining (e.g., contacting, mixing, dissolving, granulating, dragee-making, levigating, emulsifying, encapsulating, entrapping, or lyophilizing) a compound delineated herein with one or more suitable carriers, diluents, excipients, or auxiliaries, including those described herein (e.g., for pharmaceutical, agricultural, or veterinary use).

Pharmaceutical compositions of the present disclosure can take a form suitable for virtually any mode of administration, including, for example, intrathecal, topical, ocular, oral, buccal, systemic, nasal, injection, transdermal, rectal, vaginal, and the like, or a form suitable for administration by inhalation or insufflation.

Systemic formulations include those designed for administration by injection, e.g., subcutaneous, intravenous, intramuscular, intrathecal, or intraperitoneal injection, as well as those designed for transdermal, transmucosal, oral, or pulmonary administration.

Useful injectable preparations include sterile suspensions, solutions, or emulsions of the active compound(s) in aqueous or oily vehicles. The compositions also can contain formulating agents, such as suspending, stabilizing and/or dispersing agent. The formulations for injection can be presented in unit dosage form (e.g., in ampules or in multidose containers) and can contain added preservatives.

Alternatively, the injectable formulation can be provided in powder form for reconstitution with a suitable vehicle, including but not limited to, sterile pyrogen free water, buffer, dextrose solution, and the like, before use. To this end, the active compound(s) can be dried by any art-known technique, such as lyophilization, and reconstituted prior to use.

For prolonged delivery, the active compound(s), or prodrug(s) can be formulated as a depot preparation for administration by implantation or intramuscular injection. The active ingredient can be formulated with suitable polymeric or hydrophobic materials (e.g., as an emulsion in an acceptable oil) or ion exchange resins, or as sparingly soluble derivatives, e.g., as a sparingly soluble salt.

Alternatively, other pharmaceutical delivery systems can be employed. Liposomes and emulsions are well-known examples of delivery vehicles that can be used to deliver active compound(s), oligonucleotide(s), or prodrug(s). Certain organic solvents such as dimethylsulfoxide (DMSO) also can be employed.

The pharmaceutical compositions can, if desired, be presented in a pack or dispenser device that can contain one or more unit dosage forms containing the active compound(s). The pack can, for example, comprise metal or plastic foil, such as a blister pack. The pack or dispenser device can be accompanied by instructions for administration.

The active compound(s), or prodrug(s) of the present disclosure, or compositions thereof, will generally be used in an amount effective to achieve the intended result, for example in an amount effective to treat or prevent the particular disease being treated. The compound(s) and oligonucleotide(s) can be administered therapeutically to achieve therapeutic benefit or prophylactically to achieve prophylactic benefit. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient can still be afflicted with the underlying disorder. Therapeutic benefit also includes halting or slowing the progression of the disease, regardless of whether improvement is realized.

For prophylactic administration, the compound can be administered to a patient at risk of developing one of the previously described diseases. A patient at risk of developing a disease can be a patient having characteristics placing the patient in a designated group of at-risk patients, as defined by an appropriate medical professional or group. A patient at risk may also be a patient that is commonly or routinely in a setting where development of the underlying disease could occur. In other words, an at-risk patient is one who is commonly or routinely exposed to the disease or illness causing conditions or may be acutely exposed for a limited time. Alternatively, prophylactic administration can be applied to avoid the onset of symptoms in a patient diagnosed with the underlying disorder.

The amount of compound administered will depend upon a variety of factors, including, for example, the particular indication being treated, the mode of administration, whether the desired benefit is prophylactic or therapeutic, the severity of the indication being treated, the age and weight of the patient, the bioavailability of the particular active compound, and the like. Determination of an effective dosage is well within the capabilities of those skilled in the art.

Effective dosages can be estimated initially from in vitro assays. For example, an initial dosage for use in animals can be formulated to achieve a circulating blood or serum concentration of active compound that is at or above an IC₅₀ of the particular compound as measured in an in vitro assay, such as an in vitro fungal MIC or MFC, and other in vitro assays. Calculating dosages to achieve such circulating blood or serum concentrations taking into account the bioavailability of the particular compound is well within the capabilities of skilled artisans. For guidance, see “General Principles,” In: Goodman and Gilman's The Pharmaceutical Basis of Therapeutics, Chapter 1, pp. 1-112, 13th ed., McGraw-Hill, and the references cited therein, which are incorporated herein by reference.

Initial dosages also can be estimated from in vivo data, such as animal models. Animal models useful for testing the efficacy of compounds to treat or prevent the various diseases described above are well-known in the art.

Dosage amounts will typically be in the range of from about 0.0001 or 0.001 or 0.01 mg/kg/day to about 100 mg/kg/day, but can be higher or lower, depending upon, among other factors, the activity of the compound, its bioavailability, the mode of administration, and various factors discussed above. Dosage amount and interval can be adjusted individually to provide plasma levels of the compound(s) that are sufficient to maintain therapeutic or prophylactic effect. In cases of local administration or selective uptake, such as local topical administration, the effective local concentration of active compound(s) cannot be related to plasma concentration. Skilled artisans will be able to optimize effective local dosages without undue experimentation.

Preferably, the compound(s) will provide therapeutic or prophylactic benefit and will have acceptable tolerability. Tolerability of the compound(s) and oligonucleotide(s) can be determined using standard pharmaceutical procedures. The dose ratio between non-tolerable and therapeutic (or prophylactic) effect is the therapeutic index. Compounds(s) that exhibit high therapeutic indices are preferred.

### Additional Embodiments

Certain embodiments include embodiment P1 to embodiment P77 following.

Embodiment P1. A compound comprising the structure of Formula (I), or a salt thereof:

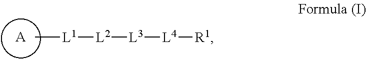

wherein

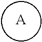

is a Tropomyosin receptor B (TrkB) ligand;
each of L₁, L₂, L₃, and L₄ is independently a linker or absent; and
R¹ is an oligonucleotide, a protecting group, a small molecule, a protein, an antibody, or a peptide.

Embodiment P2. The compound, or salt thereof, of embodiment P1, wherein the TrkB ligand is a TrkB agonist.

Embodiment P3. The compound, or salt thereof, of embodiment P1 or P2, wherein the TrkB ligand is selected from the group consisting of 3,7-dihydroxyflavone, 3,7,8,2′-tetrahydroxyflavone, 7,3′-dihydroxyflavone, 7,8,2′-trihydroxyflavone, 7,8,3′-trihydroxyflavone, 7,8,4′-trihydroxyflavone, diosmetin (5,7,3′-trihydroxy-4′-methoxyflavone), 7-hydroxy-4′-methoxyflavone, 8-hydroxy-7-methoxyflavone, eutropoflavin (4′-dimethylamino-7,8-dihydroxyflavone), norwogonin (5,7,8-trihydroxyflavone), R7, R13, tropoflavin (7,8-dihydroxyflavone), quercetin (3,3′,4′,5,7-pentahydroxyflavone), apigenin (4′,5,7-trihydroxyflavone), isocoumarin, gossypetin (3,5,7,8,3′,4′-hexahydroxyflavone), 2-methyl-8-phenylchromeno[7,8-d]imidazol-6 (3H)-one, 8-phenylchromeno[7,8-d]imidazol-6 (3H)-one, 4-oxo-2-phenyl-4H-chromene-7,8-diyl diacetate, ANA-12, an anti-TrkB antibody, and prodrugs and derivatives thereof.

Embodiment P4. The compound, or salt thereof, of any one of embodiments P1-P3, wherein the TrkB ligand is flavone, tropoflavin, or a prodrug or derivative thereof.

Embodiment P5. The compound, or salt thereof, of any one of embodiments P1-P4, wherein the compound comprises the structure of Formula (II), or a salt thereof:

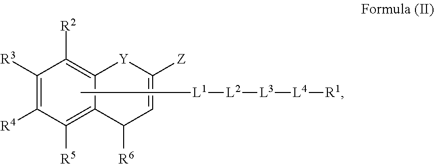

wherein
R² is hydrogen, —OH, optionally substituted —O-alkyl, optionally substituted —OAc, —NH₂, optionally substituted —NHAc, or —SH;
R³ is hydrogen, —OH, optionally substituted —O-alkyl, optionally substituted —OAc, —NH₂, optionally substituted —NHAc, or —SH;
R⁴ is hydrogen, —OH, optionally substituted —O-alkyl, optionally substituted —OAc, —NH₂, optionally substituted —NHAc, or —SH;
R⁵ is hydrogen, —OH, optionally substituted —O-alkyl, optionally substituted —OAc, —NH₂, optionally substituted —NHAc, or —SH;
R⁶ is hydrogen, —OH, optionally substituted —O-alkyl, optionally substituted —OAc, —NH₂, optionally substituted —NHAc, —SH, or ═O;
Y is CH₂, NH, S, or O; and
Z is optionally substituted aryl or optionally substituted heteroaryl.

Embodiment P6. The compound, or salt thereof, of embodiment P5, wherein the compound comprises the structure of Formula (II-a):

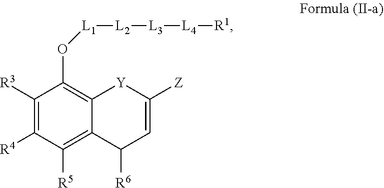

or a salt thereof.

Embodiment P7. The compound, or salt thereof, of embodiment P5, wherein the compound comprises the structure of Formula (II-b):

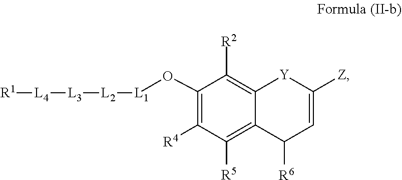

or a salt thereof.

Embodiment P8. The compound, or salt thereof, of embodiment P5, wherein the compound comprises the structure of Formula (II-c):

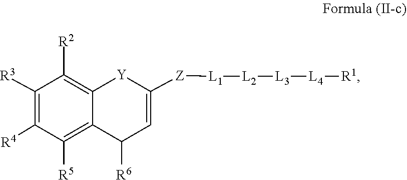

or a salt thereof.

Embodiment P9. The compound, or salt thereof, of any one of embodiments P5-P8, wherein R² is —OH.

Embodiment P10. The compound, or salt thereof, of any one of embodiments P5-P9, wherein R³ is —OH.

Embodiment P11. The compound, or salt thereof, of any one of embodiments P5-P10, wherein R⁴ is hydrogen.

Embodiment P12. The compound, or salt thereof, of any one of embodiments P5-P11, wherein R⁵ is hydrogen.

Embodiment P13. The compound, or salt thereof, of any one of embodiments P5-P12, wherein R⁶ is ═O.

Embodiment P14. The compound, or salt thereof, of any one of embodiments P5-P13, wherein Y is O.

Embodiment P15. The compound, or salt thereof, of any one of embodiments P5-P14, wherein Z is optionally substituted aryl.

Embodiment P16. The compound, or salt thereof, of any one of embodiments P5-P15, wherein Z is optionally substituted phenyl.

Embodiment P17. The compound, or salt thereof, of any one of embodiments P5-P16, wherein Z is unsubstituted phenyl.

Embodiment P18. The compound, or salt thereof, of any one of embodiments P5-P16, wherein Z is phenyl substituted with one or more —OH and/or —O-alkyl.

Embodiment P19. The compound, or salt thereof, of embodiment P5, wherein the compound comprises the structure of Formula (III):

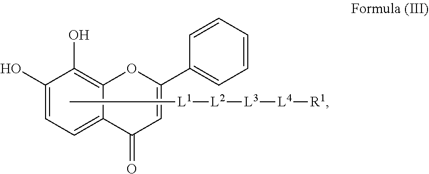

or a salt thereof.

Embodiment P20. The compound, or salt thereof, of embodiment P19, wherein the compound comprises the structure of Formula (III-a):

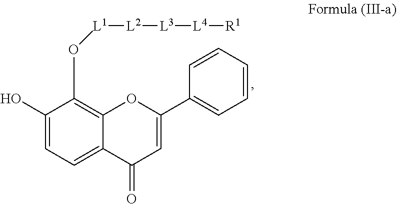

or a salt thereof.

Embodiment P21. The compound, or salt thereof, of embodiment P19, wherein the compound comprises the structure of Formula (III-b):

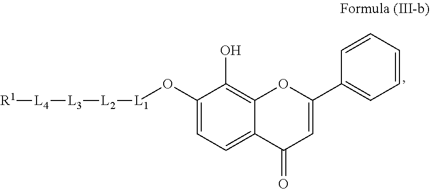

or a salt thereof.

Embodiment P22. The compound, or salt thereof, of embodiment P19, wherein the compound comprises the structure of Formula (III-c):

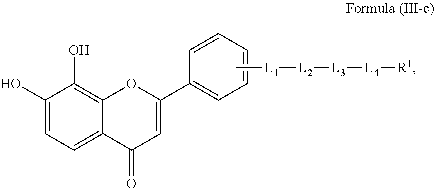

or a salt thereof.

Embodiment P23. The compound, or salt thereof, of embodiment P22, wherein the compound comprises the structure of Formula (III-c-1):

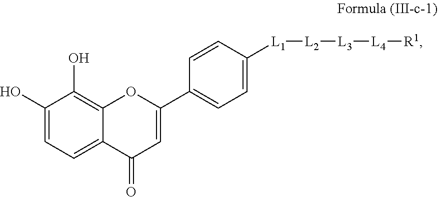

or a salt thereof.

Embodiment P24. The compound, or salt thereof, of any one of embodiments P1-P3, wherein the TrkB ligand is ANA-12, or a prodrug or derivative thereof.

Embodiment P25. The compound, or salt thereof, of any one of embodiments P1-P3 or P24, wherein the compound comprises the structure of Formula (IV):

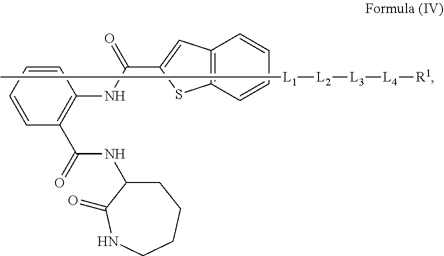

or a salt thereof.

Embodiment P26. The compound, or salt thereof, of embodiment P25, wherein the compound comprises the structure of Formula (IV-a):

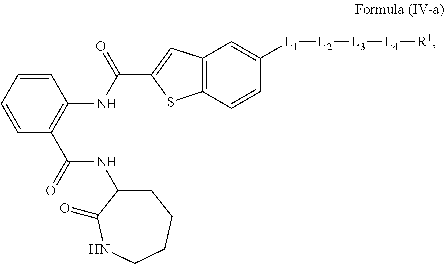

or a salt thereof.

Embodiment P27. The compound, or salt thereof, of embodiment P25, wherein the compound comprises the structure of Formula (IV-b):

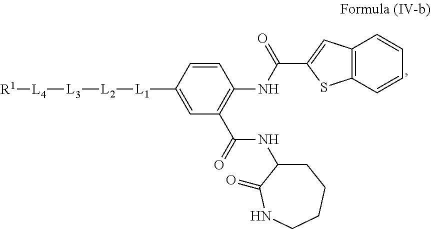

or a salt thereof.

Embodiment P28. The compound, or salt thereof, of any one of embodiments P1-P27, wherein each of L₁, L₂, L₃, and L₄ is independently absent, a bond, an optionally substituted alkyl linker, an optionally substituted polyethylene glycol (PEG) linker, an optionally substituted heteroalkyl linker, or an optionally substituted heteroaryl linker.

Embodiment P29. The compound, or salt thereof, of embodiment P28, wherein L₁ is an optionally substituted heteroaryl linker.

Embodiment P30. The compound, or salt thereof, of embodiment P29, wherein L₁ is an optionally substituted unsaturated heteroaryl linker.

Embodiment P31. The compound, or salt thereof, of embodiment P29 or P30, wherein L₁ comprises the structure

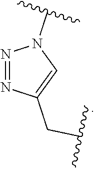 [SMILES: *Cc1cn(*)nn1]

Embodiment P32. The compound, or salt thereof, of embodiment P28, wherein L₁ is an optionally substituted heteroalkyl linker.

Embodiment P33. The compound, or salt thereof, of embodiment P32, wherein the optionally substituted heteroalkyl linker is an optionally substituted C₁-C₁₀ alkyl chain in which one or more carbon atoms are replaced with O, N, or S.

Embodiment P34. The compound, or salt thereof, of embodiment P32 or P33, wherein L₁ comprises the structure

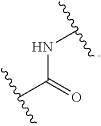 [SMILES: *NC(*)=O]

Embodiment P35. The compound, of salt thereof, of embodiment P32 or P33, wherein L¹ comprises the structure

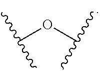 [SMILES: *O*]

Embodiment P36. The compound, or salt thereof, of any one of embodiments P28-P35, wherein L₂ is an optionally substituted PEG linker.

Embodiment P37. The compound, or salt thereof, of embodiment P36, wherein the PEG linker is five PEG units in length.

Embodiment P38. The compound, or salt thereof, of embodiment P36, wherein the PEG linker is four PEG units in length.

Embodiment P39. The compound, of salt thereof, of embodiment P36, wherein the PEG linker is three PEG units in length.

Embodiment P40. The compound, or salt thereof, of any one of embodiments P28-P35, wherein L₂ is an optionally substituted alkyl linker.

Embodiment P41. The compound, or salt thereof, of embodiment P40, wherein L₂ is an optionally substituted C₁₋₂₀ alkyl linker.

Embodiment P42. The compound, or salt thereof, of embodiment P40 or P41, wherein L₂ is an optionally substituted C₈ alkyl linker.

Embodiment P43. The compound, or salt thereof, of any one of embodiments P28-P42, wherein L₃ is an optionally substituted heteroaryl linker.

Embodiment P44. The compound, or salt thereof, of embodiment P43, wherein L₃ is an optionally substituted partially unsaturated heteroaryl linker.

Embodiment P45. The compound, or salt thereof, of embodiment P44, wherein L₃ comprises the structure:

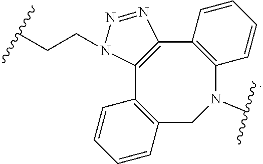 [SMILES: *CCn1nnc2c1-c1ccccc1CN(*)c1ccccc1-2]

Embodiment P46. The compound, or salt thereof, of any one of embodiments P28-P45, wherein L₄ is an optionally substituted heteroalkyl linker.

Embodiment P47. The compound, or salt thereof, of embodiment P46, wherein the heteroalkyl linker is substituted with one or more ═O substituents.

Embodiment P48. The compound, or salt thereof, of embodiment P46 or P47, wherein the heteroalkyl linker comprises two substituents joined together to form an optionally substituted carbocyclyl ring.

Embodiment P49. The compound, or salt thereof, of embodiment P46 or P47, wherein L₄ comprises the structure:

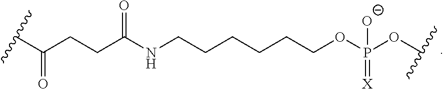

wherein X is O or S.

Embodiment P50. The compound, or salt thereof, of any one of embodiments P46-P48, wherein L₄ comprises the structure:

wherein X is O or S.

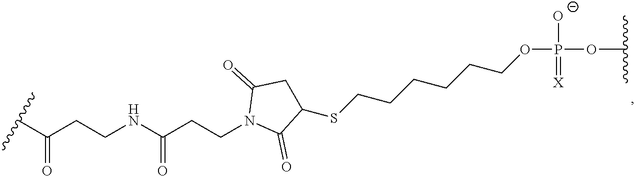

Embodiment P51. The compound, or salt thereof, of embodiment P28, wherein L₁, L₂, L₃, and L₄ together comprise the structure

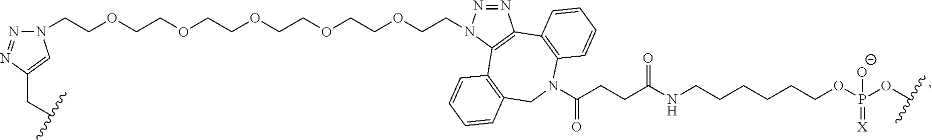

wherein X is O or S.

Embodiment P52. The compound, or salt thereof, of embodiment P28, wherein L₁, L₂, L₃, and L₄ together comprise the structure

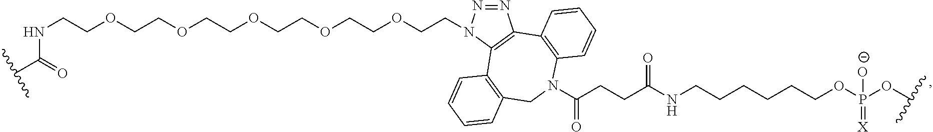

wherein X is O or S.

Embodiment P53. The compound, or salt thereof, of embodiment P28, wherein L₁, L₂, L₃, and L₄ together comprise the structure:

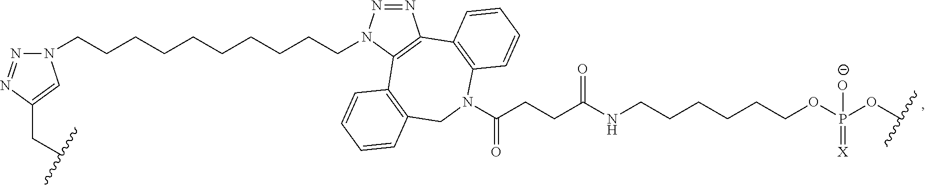

wherein X is O or S.

Embodiment P54. The compound, or salt thereof, of embodiment P28, wherein L₁, L₂, L₃, and L₄ together comprise the structure

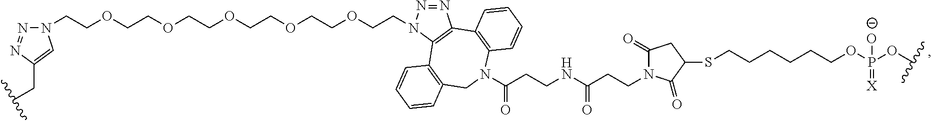

wherein X is O or S.

Embodiment P55. The compound, or salt thereof, of embodiment P28, wherein L₁, L₂, L₃, and L₄ together comprise the structure:

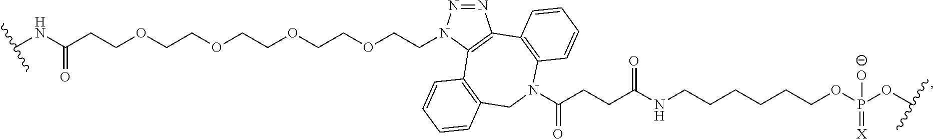

wherein X is O or S.

Embodiment P56. The compound, or salt thereof, of embodiment P28, wherein L₁, L₂, L₃, and L₄ together comprise the structure:

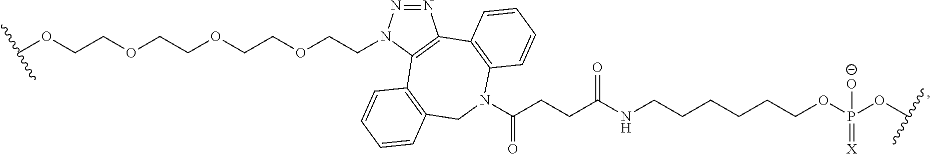

wherein

### X is O or S.

Embodiment P57. The compound, or salt thereof, of any one of embodiments P1-P56, wherein the compound comprises the structure:

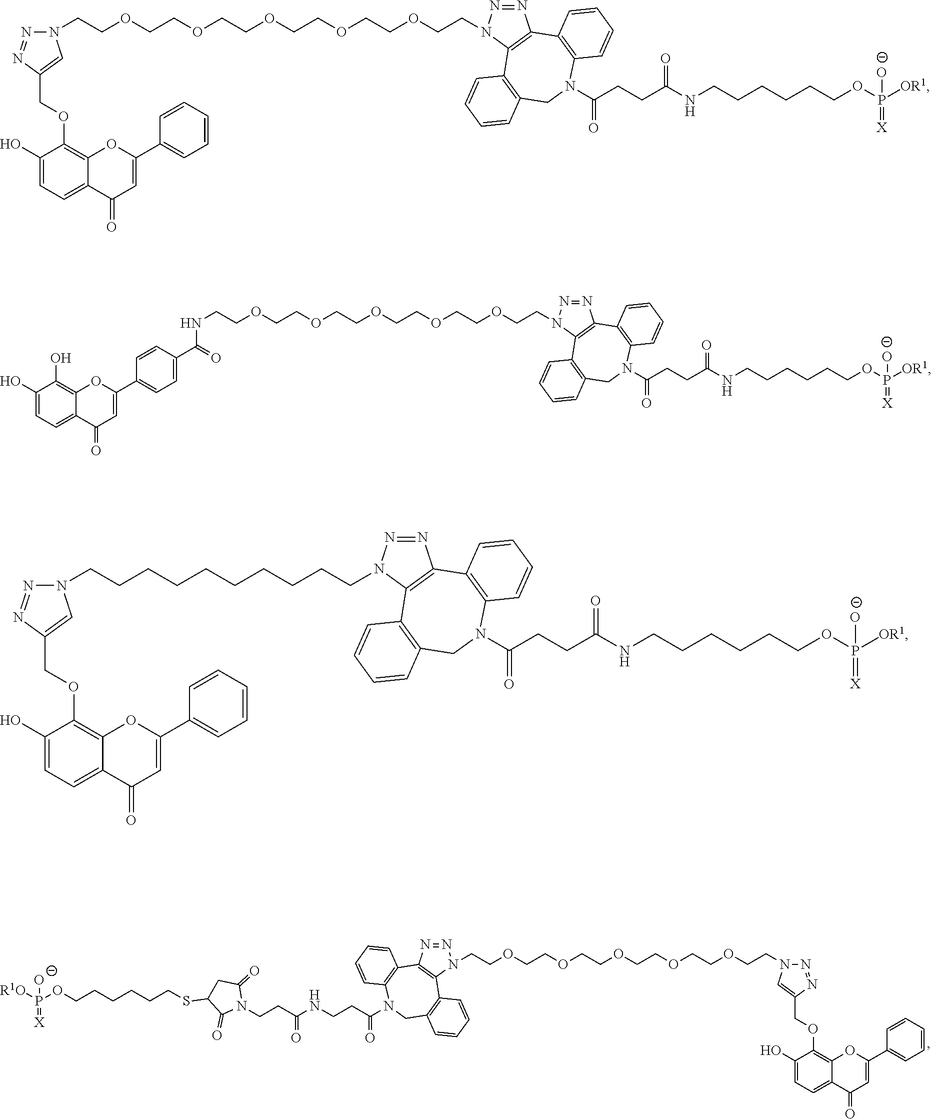

or a salt thereof, wherein X is O or S.

Embodiment P58. The compound, or salt thereof, of any one of embodiments P1-P56, wherein the compound comprises the structure:

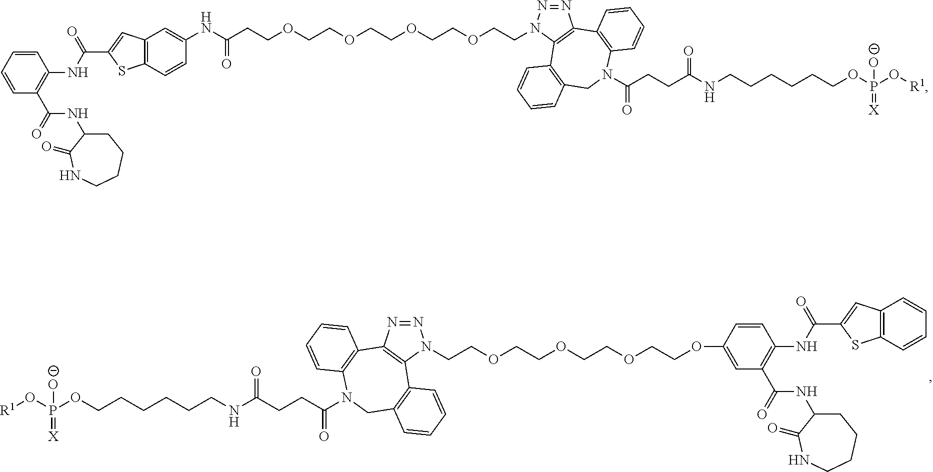

or a salt thereof, wherein X is O or S.

Embodiment P59. The compound, or salt thereof, of any one of embodiments P49-P58, wherein X is O.

Embodiment P60. The compound, or salt thereof, of any one of embodiments P49-P58, wherein X is S.

Embodiment P61. The compound, or salt thereof, of any one of embodiments P1-P60, wherein R¹ comprises an oligonucleotide.

Embodiment P62. The compound, or salt thereof, of embodiment P61, wherein the oligonucleotide is attached at its 5′ end.

Embodiment P63. The compound, or salt thereof, of embodiment P61, wherein the oligonucleotide is attached at its 3′ end.

Embodiment P64. The compound, or salt thereof, of embodiment P61, wherein the oligonucleotide is attached at an internal position on the oligonucleotide.

Embodiment P65. The compound, or salt thereof, of embodiment P64, wherein the internal position is an internucleoside linkage.

Embodiment P66. The compound, or salt thereof, of any one of embodiments P1-P65, wherein R¹ comprises an oligonucleotide conjugated to one or more additional TrkB ligands.

Embodiment P67. The compound, or salt thereof, of embodiment P66, wherein the oligonucleotide is conjugated to two, three, four, five, or more than five additional TrkB ligands.

Embodiment P68. The compound, or salt thereof, of embodiment P66 or P67, wherein the additional TrkB ligands are conjugated to the oligonucleotide at the 5′ end of the oligonucleotide, the 3′ end of the oligonucleotide, one or more internal positions on the oligonucleotide, or any combination thereof.

Embodiment P69. The compound, or salt thereof, of any one of embodiments P61-P68, wherein the oligonucleotide is a modified oligonucleotide.

Embodiment P70. A composition comprising a compound, or salt thereof, of any one of embodiments P1-P69, and a pharmaceutically acceptable excipient.

Embodiment P71. A method for delivering a therapeutic oligonucleotide to the brain of a subject, comprising administration of a compound, or salt thereof, of any one of embodiments P1-P69, or a composition of embodiment P70, to the subject.

Embodiment P72. The method of embodiment P71, wherein the therapeutic oligonucleotide is delivered to one or more brain regions selected from the group consisting of the striatum, the cerebellum, the brain stem, the hippocampus, the frontal cortex, and the spinal cord.

Embodiment P73. A method for treating or ameliorating a disease, disorder, or symptom thereof in a subject, comprising administration of a compound, or salt thereof, of any one of embodiments P1-P69, or a composition of embodiment P70, to the subject.

Embodiment P74. The method of embodiment P73, wherein the disease, disorder, or symptom thereof is a central nervous system (CNS) disease, disorder, or symptom thereof.

Embodiment P75. The method of embodiment P73 or P74, wherein the disease, disorder, or symptom thereof is Alzheimer's disease, or a symptom thereof.

Embodiment P76. The method of any one of embodiments P71-P75, wherein the compound, or salt thereof, is administered to the subject intrathecally.

Embodiment P77. A method for making a compound, or salt thereof, of any one of embodiments P1-P69, comprising one or more compounds and chemical transformations described herein, including Example 1.

Additional embodiments include embodiment 1 to embodiment 135 following.

Embodiment 1. A compound, or a stereoisomer, tautomer, prodrug, or salt thereof, comprising the structure of Formula (V):

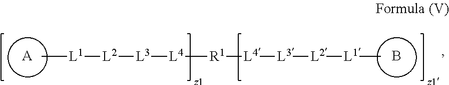

wherein:
each

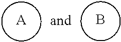

are independently a Tropomyosin receptor B (TrkB) ligand;
each of L¹, L², L³, L⁴, L^{1′}, L^{2′}, L^{3′}, and L^{4′} is independently a linker, bond, or absent;
R¹ is an oligonucleotide, a protecting group, a small molecule, a protein, an antibody, or a peptide;
z1 is 1, 2, or 3; and
z1′ is 0, 1, 2, or 3.

Embodiment 2. The compound of embodiment 1, or a stereoisomer, tautomer, prodrug, or salt thereof, wherein:
z1 is 1; and
z1′ is 0 or 1.

Embodiment 3. The compound, or a stereoisomer, tautomer, prodrug, or salt thereof, of embodiment 2, wherein z1 is 1 and z1′ is 0.

Embodiment 4. The compound, or a stereoisomer, tautomer, prodrug, or salt thereof, of any one of embodiments 1-3, wherein the compound comprises the structure of Formula (I):

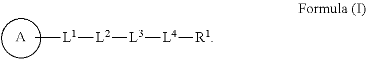

Embodiment 5. The compound, or a stereoisomer, tautomer, prodrug, or salt thereof, of embodiment 4, comprising the structure of Formula (I′):

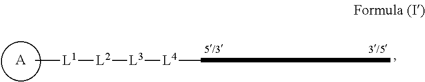

wherein:
[symbol] is an oligonucleotide.

Embodiment 6. The compound, or a stereoisomer, tautomer, prodrug, or salt thereof, of embodiment 1 or 2, wherein z1 and z1′ are each independently 1.

Embodiment 7. The compound, or a stereoisomer, tautomer, prodrug, or salt thereof, of embodiment 6, wherein the compound comprises the structure of Formula (VI):

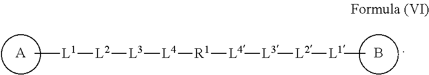

Embodiment 8. The compound, or a stereoisomer, tautomer, prodrug, or salt thereof, of embodiment 7, wherein the compound comprises the structure of Formula (VI′):

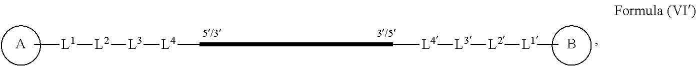

wherein:
[symbol] is an oligonucleotide.

Embodiment 9. The compound, or a stereoisomer, tautomer, prodrug, or salt thereof, of any one of embodiments 1-8, wherein the TrkB ligand comprises a substituted or unsubstituted aryl, substituted or unsubstituted heteroaryl, substituted or unsubstituted heterocycle, substituted or unsubstituted heterocycloalkyl, or substituted or unsubstituted cycloalkyl.

Embodiment 10. The compound, or a stereoisomer, tautomer, prodrug, or salt thereof, of any one of embodiments 1-9, wherein the TrkB ligand comprises a substituted or unsubstituted aryl, substituted or unsubstituted heteroaryl, or substituted or unsubstituted heterocycloalkyl.

Embodiment 11. The compound, or a stereoisomer, tautomer, prodrug, or salt thereof, of any one of embodiments 1-10, wherein the TrkB ligand comprises a substituted or unsubstituted mono- or polycyclic aryl, substituted or unsubstituted mono- or polycyclic heteroaryl, substituted or unsubstituted mono- or polycyclic heterocycloalkyl, or substituted or unsubstituted mono- or polycyclic cycloalkyl.

Embodiment 12. The compound, or a stereoisomer, tautomer, prodrug, or salt thereof, of embodiment 11, wherein the substituted or unsubstituted polycyclic aryl, substituted or unsubstituted polycyclic heteroaryl, substituted or unsubstituted polycyclic heterocycloalkyl or substituted or unsubstituted mono- or polycyclic cycloalkyl are a fused ring system.

Embodiment 13. The compound, or a stereoisomer, tautomer, prodrug, or salt thereof, of embodiment 12, wherein the fused ring system comprises two to four fused rings.

Embodiment 14. The compound, or a stereoisomer, tautomer, prodrug, or salt thereof, of embodiment 13, wherein the fused ring system further comprises an optionally substituted pendant cycloalkyl, optionally substituted pendant heterocycloalkyl, optionally substituted pendant aryl, or an optionally substituted pendant heteroaryl.

Embodiment 15. The compound, or a stereoisomer, tautomer, prodrug, or salt thereof, of any one of embodiments 1-14, wherein each TrkB ligand is independently:

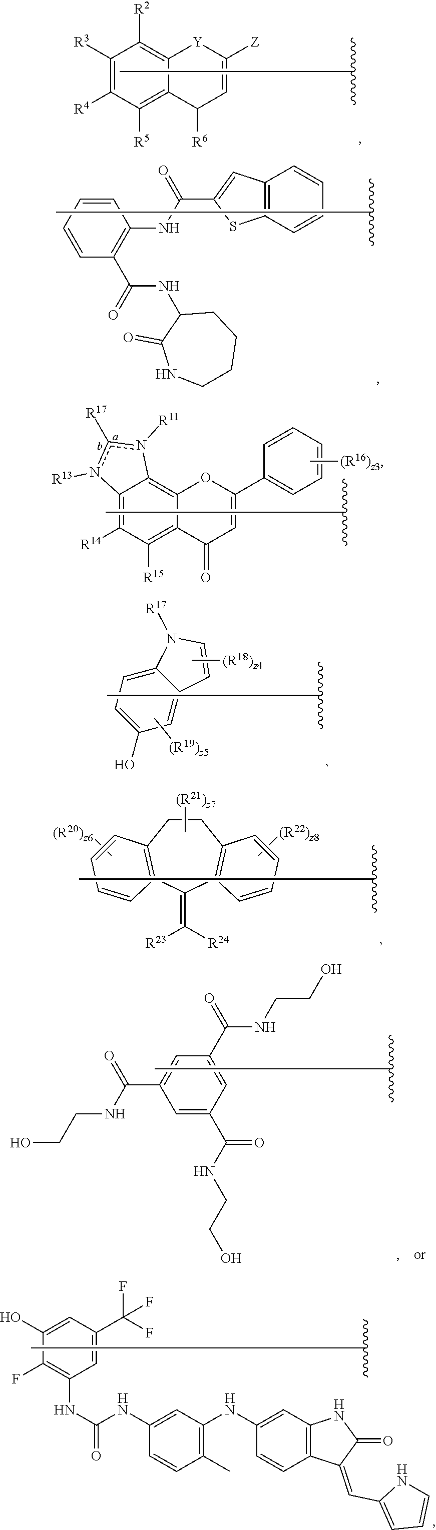

wherein:
R² is hydrogen, —OR⁷, —SR⁸, or —NR⁹R¹⁰;
R³ is hydrogen, —OR³¹, —SR³², or —NR³³R³⁴;
R⁴ is hydrogen, —OR³⁵, —SR³⁶, or —NR³⁷R³⁸,
R⁵ is hydrogen, —OR³⁹, —SR⁴⁰, or —NR⁴¹R⁴².
R⁶ is hydrogen, —OH, optionally substituted —O-alkyl, optionally substituted —OAc, —NH₂, optionally substituted —NHAc, —SH, or ═O;
R⁷, R⁸, R⁹, R¹⁰, R³¹, R³², R³³, R³⁴, R³⁵, R³⁶, R³⁷, R³⁸, R³⁹, R⁴⁰, R⁴¹, and R⁴² are each independently hydrogen, optionally substituted alkyl, optionally substituted heteroalkyl, optionally substituted cycloalkyl, optionally substituted heterocycloalkyl, optionally substituted aryl, optionally substituted heteroaryl;
Y is CH₂, NH, S, or O;
Z is optionally substituted aryl or optionally substituted heteroaryl;
R¹¹ and R₁₃ are each independently absent, hydrogen, or optionally substituted alkyl;
R¹², R¹⁴, and R¹⁵ are each independently hydrogen, optionally substituted alkyl, optionally substituted heteroalkyl, optionally substituted cycloalkyl, optionally substituted heterocycloalkyl, optionally substituted aryl, or optionally substituted heteroaryl;
R¹⁶ is hydrogen, halogen, —CN, —N₃, —SOₙ₁₆R^{1A}, —SOᵥ₁₆NR^{16B}R^{16C}, —NHNR^{16B}R^{16C}, —ONR^{16B}R^{16C}, —NHC(O)NHNR^{16B}R^{16C}, —NHC(O)NR^{16B}R^{16C}, —N(O)ₘ₁₆, —NR^{16B}R^{16C}, —C(O)R^{16D}, —C(O)OR^{16D}, —C(O)NR^{16B}R^{16C}, —OR^{16A}, —NR^{16B}SO₂R^{16A}, —NR^{16B}C(O)R^{16D};
—NR^{16B}C(O)OR^{16D}, —NR^{16B}OR^{16D}, optionally substituted alkyl, optionally substituted heteroalkyl, optionally substituted cycloalkyl, optionally substituted heterocycloalkyl, optionally substituted aryl, or optionally substituted heteroaryl;
[symbol] and [symbol] are each independently a single bond or a double bond, wherein if [symbol] is a single bond, then [symbol] is a double bond and R¹³ is absent; and further wherein if [symbol] is a single bond, then [symbol] is a double bond and R¹¹ is absent;
R^{16A}, R^{16B}, R^{16C}, R^{16D} are each independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{16B} and R^{16C} substituents bonded to the same nitrogen atom may optionally be joined to form a substituted or unsubstituted heterocycloalkyl or substituted or unsubstituted heteroaryl;
R¹⁷, R¹⁸, and R¹⁹ are each independently hydrogen, optionally substituted alkyl, optionally substituted heteroalkyl, optionally substituted cycloalkyl, optionally substituted heterocycloalkyl, optionally substituted aryl, or optionally substituted heteroaryl;
R²⁰ is hydrogen, halogen, —CN, —N₃, —SOₙ₂₀R^{1A}, —SOᵥ₂₀NR^{20B}R^{20C}, —NHNR^{20B}R^{20C}, —ONR^{20B}R^{20C}, —NHC(O)NHNR^{20B}R^{20C}, —NHC(O)NR^{20B}R^{20C}, —N(O)ₘ₂₀, —NR^{20B}R^{20C}, —C(O)R^{20D}, —C(O)OR^{20D}, —C(O)NR^{20B}R^{20C}, —OR^{20A}, —NR^{20B}SO₂R^{20A}, —NR^{20B}C(O)R^{20D};
—NR^{20B}C(O)OR^{20D}, —NR^{20B}OR^{20D}, optionally substituted alkyl, optionally substituted heteroalkyl, optionally substituted cycloalkyl, optionally substituted heterocycloalkyl, optionally substituted aryl, or optionally substituted heteroaryl;
R²¹ is hydrogen, halogen, —CN, —N₃, —SOₙ₂₁R^{1A}, —SOᵥ₂₁NR^{21B}R^{21C}, —NHNR^{21B}R^{21C}, —ONR^{21B}R^{21C}, —NHC(O)NHNR^{21B}R^{21C}, —NHC(O)NR^{21B}R^{21C}, —N(O)ₘ₂₁, —NR^{21B}R^{21C}, —C(O)R^{21D}, —C(O)OR^{21D}, —C(O)NR^{21B}R^{21C}, —OR^{21A}, —NR^{21B}SO₂R^{21A}, —NR^{21B}C(O)R^{21D};
—NR^{21B}C(O)OR^{21D}, —NR^{21B}OR^{21D}, optionally substituted alkyl, optionally substituted heteroalkyl, optionally substituted cycloalkyl, optionally substituted heterocycloalkyl, optionally substituted aryl, or optionally substituted heteroaryl;
R²² and R²³ are each independently hydrogen, optionally substituted alkyl, optionally substituted heteroalkyl, optionally substituted cycloalkyl, optionally substituted heterocycloalkyl, optionally substituted aryl, or optionally substituted heteroaryl;
R²⁴ is hydrogen, halogen, —CN, —N₃, —SOₙ₂₄R^{1A}, —SOᵥ₂₄NR^{24B}R^{24C}, —NHNR^{24B}R^{24C}, —ONR^{24B}R^{24C}, —NHC(O)NHNR^{24B}R^{24C}, —NHC(O)NR^{24B}R^{24C}, —N(O)ₘ₂₄, —NR^{24B}R^{24C}, —C(O)R^{24D}, —C(O)OR^{24D}, —C(O)NR^{24B}R^{24C}, —OR^{24A}, —NR^{24B}SO₂R^{24A}, —NR^{24B}C(O)R^{24D};
—NR^{24B}C(O)OR^{24D}, —NR^{24B}OR^{24D}, optionally substituted alkyl, optionally substituted heteroalkyl, optionally substituted cycloalkyl, optionally substituted heterocycloalkyl, optionally substituted aryl, or optionally substituted heteroaryl;
R^{20A}, R^{20B}, R^{20C}, R^{20D}, R^{21A}, R^{21B}, R^{21C}, R^{21D}, R^{24A}, R^{24B}, R^{24C}, and R^{24D} are each independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{20B}, R^{20C}, R^{21B}, R^{21C}, R^{24B}, R^{24C}, R^{24B}, and R^{24C} substituents bonded to the same nitrogen atom may optionally be joined to form a substituted or unsubstituted heterocycloalkyl or substituted or unsubstituted heteroaryl;
n16, n20, n21, n23, n24, z6, and z8 are each independently 0, 1, 2, 3, or 4;
v16, v20, v21, m16, m20, m21, and m24 are each independently 1 or 2;
z3 is 0, 1, 2, 3, 4, or 5;
z4 and z7 are each independently 0, 1, or 2;
z5 is 0, 1, 2, or 3; and
z6 and z8 are each independently 0, 1, 2, 3, or 4.

Embodiment 16. The compound, or a stereoisomer, tautomer, prodrug, or salt thereof, of any one of embodiments 1-15, wherein each TrkB ligand is independently flavone, tropoflavin, or derivatives thereof.

Embodiment 17. The compound, or a stereoisomer, tautomer, prodrug, or salt thereof, of any one of embodiments 1-16, wherein each TrkB ligand is independently 3,7-dihydroxyflavone, 3,7,8,2′-tetrahydroxyflavone, 7,3′-dihydroxyflavone, 7,8,2′-trihydroxyflavone, 7,8,3′-trihydroxyflavone, 7,8,4′-trihydroxyflavone, diosmetin (5,7,3′-trihydroxy-4′-methoxyflavone), 7-hydroxy-4′-methoxyflavone, 8-hydroxy-7-methoxyflavone, eutropoflavin (4′-dimethylamino-7,8-dihydroxyflavone), norwogonin (5,7,8-trihydroxyflavone), R7, R13, tropoflavin (7,8-dihydroxyflavone), quercetin (3,3′,4′,5,7-pentahydroxyflavone), apigenin (4′,5,7-trihydroxyflavone), isocoumarin, gossypetin (3,5,7,8,3′,4′-hexahydroxyflavone), 2-methyl-8-phenylchromeno[7,8-d]imidazol-6 (3H)-one, 8-phenylchromeno[7,8-d]imidazol-6 (3H)-one, 4-oxo-2-phenyl-4H-chromene-7,8-diyl diacetate, ANA-12, an anti-TrkB antibody, or derivatives thereof

Embodiment 18. A compound, or a stereoisomer, tautomer, prodrug, or salt thereof, comprising the structure of Formula (II):

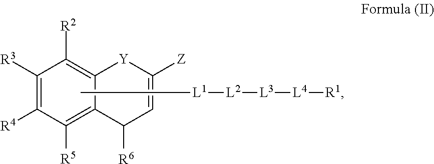

wherein:
R² is hydrogen, —OR⁷, —SR⁸, or —NR⁹R¹⁰;
R³ is hydrogen, —OR³¹, —SR³², or —NR³³R³⁴;
R⁴ is hydrogen, —OR³⁵, —SR³⁶, or —NR³⁷R³⁸;
R⁵ is hydrogen, —OR³⁹, —SR⁴⁰, or —NR⁴¹R⁴²;
R⁶ is hydrogen, —OH, optionally substituted —O-alkyl, optionally substituted —OAc, —NH₂, optionally substituted —NHAc, —SH, or ═O;
R⁷, R⁸, R⁹, R¹⁰, R³¹, R³², R³³, R³⁴, R³⁵, R³⁶, R³⁷, R³⁸, R³⁹, R⁴⁰, R⁴¹, and R⁴² are each independently hydrogen, optionally substituted alkyl, optionally substituted heteroalkyl, optionally substituted cycloalkyl, optionally substituted heterocycloalkyl, optionally substituted aryl, optionally substituted heteroaryl;
Y is CH₂, NH, S, or O; and
Z is optionally substituted aryl or optionally substituted heteroaryl.

Embodiment 19. The compound, or a stereoisomer, tautomer, prodrug, or salt thereof, of embodiment 18, wherein the compound comprises the structure of Formula (II-a):

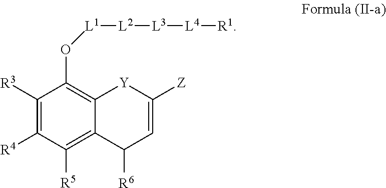

Embodiment 20. The compound, or a stereoisomer, tautomer, prodrug, or salt thereof, of embodiment 18, wherein the compound comprises the structure of Formula (II-b):

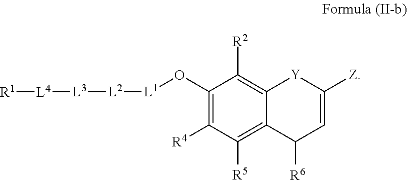

Embodiment 21. The compound, or a stereoisomer, tautomer, prodrug, or salt thereof, of embodiment 18, wherein the compound comprises the structure of Formula (II-c):

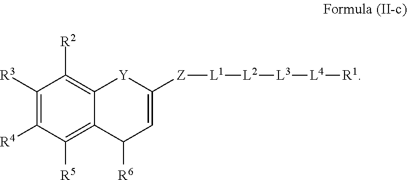

Embodiment 22. The compound, or a stereoisomer, tautomer, prodrug, or salt thereof, of any one of embodiments 15-21, wherein R² is —OH.

Embodiment 23. The compound, or a stereoisomer, tautomer, prodrug, or salt thereof, of any one of embodiments 15-22, wherein R³ is —OH.

Embodiment 24. The compound, or a stereoisomer, tautomer, prodrug, or salt thereof, of any one of embodiments 15-23, wherein R⁴ is hydrogen.

Embodiment 25. The compound, or a stereoisomer, tautomer, prodrug, or salt thereof, of any one of embodiments 15-24, wherein R⁵ is hydrogen.

Embodiment 26. The compound, or a stereoisomer, tautomer, prodrug, or salt thereof, of any one of embodiments 15-25, wherein R⁶ is ═O.

Embodiment 27. The compound, or a stereoisomer, tautomer, prodrug, or salt thereof, of any one of embodiments 15-26, wherein Y is O.

Embodiment 28. The compound, or a stereoisomer, tautomer, prodrug, or salt thereof, of any one of embodiments 15-27, wherein Z is optionally substituted aryl.

Embodiment 29. The compound, or a stereoisomer, tautomer, prodrug, or salt thereof, of any one of embodiments 15-28, wherein Z is optionally substituted phenyl.

Embodiment 30. The compound, or a stereoisomer, tautomer, prodrug, or salt thereof, of any one of embodiments 15-29, wherein Z is unsubstituted phenyl.

Embodiment 31. The compound, or a stereoisomer, tautomer, prodrug, or salt thereof, of any one of embodiments 15-29, wherein Z is phenyl substituted with one or more —OH and/or —O-alkyl.

Embodiment 32. A compound, or a stereoisomer, tautomer, prodrug, or salt thereof, wherein the compound comprises the structure of Formula (III):

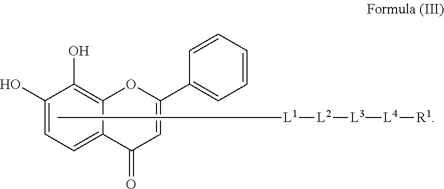

Embodiment 33. The compound, or a stereoisomer, tautomer, prodrug, or salt thereof, of embodiment 32, wherein the compound comprises the structure of Formula (III-a):

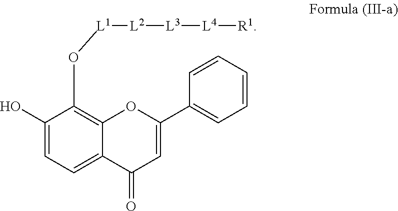

Embodiment 34. The compound, or a stereoisomer, tautomer, prodrug, or salt thereof, of embodiment 32, wherein the compound comprises the structure of Formula (III-b):

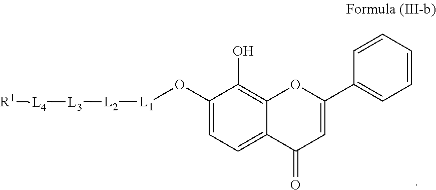

Embodiment 35. The compound, or a stereoisomer, tautomer, prodrug, or salt thereof, of embodiment 32, wherein the compound comprises the structure of Formula (III-c):

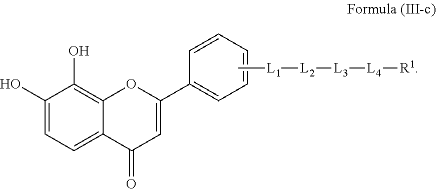

Embodiment 36. The compound, or a stereoisomer, tautomer, prodrug, or salt thereof, of embodiment 35, wherein the compound comprises the structure of Formula (III-c-1):

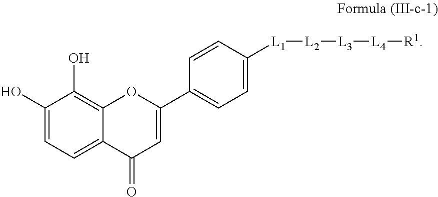

Embodiment 37. The compound, or a stereoisomer, tautomer, prodrug, or salt thereof, of any one of embodiments 1-36, wherein the TrkB ligand is ANA-12, or a prodrug or derivative thereof

Embodiment 38. A compound, or a stereoisomer, tautomer, prodrug, or salt thereof, wherein the compound comprises the structure of Formula (II′):

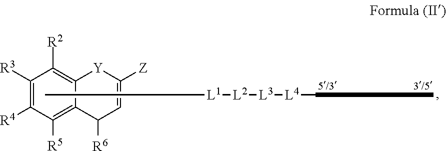

wherein:
each of L¹, L², L³, and L⁴ is independently a linker, bond, or absent;
R² is hydrogen, —OR⁷, —SR⁸, or —NR⁹R¹⁰;
R³ is hydrogen, —OR³¹, —SR³², or —NR³³R³⁴;
R⁴ is hydrogen, —OR³⁵, —SR³⁶, or —NR³⁷R³⁸;
R⁵ is hydrogen, —OR³⁹, —SR⁴⁰, or —NR⁴¹R⁴²;
R⁶ is hydrogen, —OH, optionally substituted —O-alkyl, optionally substituted —OAc, —NH₂, optionally substituted —NHAc, —SH, or ═O;
R⁷, R⁸, R⁹, R¹⁰, R³¹, R³², R³³, R³⁴, R³⁵, R³⁶, R³⁷, R³⁸, R³⁹, R⁴⁰, R⁴¹, and R⁴² are each independently hydrogen, optionally substituted alkyl, optionally substituted heteroalkyl, optionally substituted cycloalkyl, optionally substituted heterocycloalkyl, optionally substituted aryl, optionally substituted heteroaryl;
Y is CH₂, NH, S, or O; and
Z is optionally substituted aryl or optionally substituted heteroaryl; and
[symbol] is an oligonucleotide.

Embodiment 39. A compound, or a stereoisomer, tautomer, prodrug, or salt thereof, wherein the compound comprises the structure of Formula (II″):

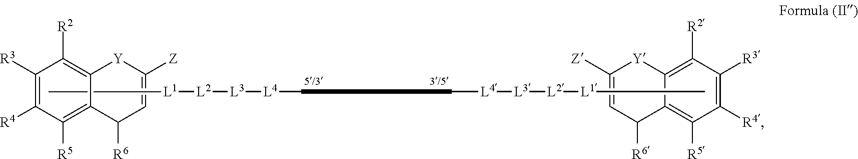

wherein:
[symbol] is an oligonucleotide;
each of L¹, L², L³, L⁴, L^{1′}, L^{2′}, L^{3′}, and L^{4′} is independently a linker, bond, or absent;
R² is hydrogen, —OR⁷, —SR⁸, or —NR⁹R¹⁰;
R^{2′} is hydrogen, —OR^{7′}, —SR^{8′}, or —NR^{9′}R^{10′};
R³ is hydrogen, —OR³¹, —SR³², or —NR³³R³⁴;
R^{3′} is hydrogen, —OR^{31′}, —SR^{32′}, or —NR^{33′}R^{34′};
R⁴ is hydrogen, —OR³⁵, —SR³⁶, or —NR³⁷R³⁸,
R^{4′} is hydrogen, —OR^{35′}, —SR^{36′}, or —NR^{37′}R^{38′},
R⁵ is hydrogen, —OR³⁹, —SR⁴⁰, or —NR⁴¹R⁴²;
R^{5′} is hydrogen, —OR^{39′}, —SR^{40′}, or —NR^{41′}R^{42′};
R⁶ is hydrogen, —OH, optionally substituted —O-alkyl, optionally substituted —OAc, —NH₂, optionally substituted —NHAc, —SH, or ═O;
R^{6′} is hydrogen, —OH, optionally substituted —O-alkyl, optionally substituted —OAc, —NH₂, optionally substituted —NHAc, —SH, or ═O;
R⁷, R^{7′}, R⁸, R^{8′}, R⁹, R^{9′}, R¹⁰, R^{10′}, R³¹, R^{31′}, R³², R^{32′}, R³³, R^{33′}, R³⁴, R^{34′}, R³⁵, R^{35′}, R³⁶, R^{36′}, R³⁷, R^{37′}, R³⁸, R^{38′}, R³⁹, R^{39′}, R⁴⁰, R^{40′}, R⁴¹, R^{41′}, R⁴², and R^{42′} are each independently hydrogen, optionally substituted alkyl, optionally substituted heteroalkyl, optionally substituted cycloalkyl, optionally substituted heterocycloalkyl, optionally substituted aryl, optionally substituted heteroaryl;
Y and Y′ are each independently CH₂, NH, S, or O; and
Z and Z′ are each independently optionally substituted aryl or optionally substituted heteroaryl.

Embodiment 40. A compound, or a stereoisomer, tautomer, prodrug, or salt thereof, comprising the structure of Formula (IV):

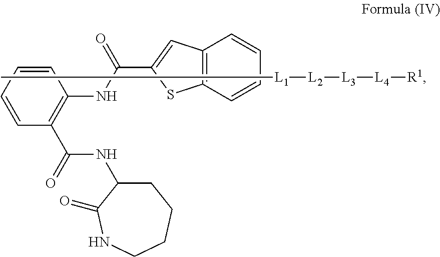

wherein each of L¹, L², L³, and L⁴ is independently a linker, bond, or absent; and
R¹ is an oligonucleotide, a protecting group, a small molecule, a protein, an antibody, or a peptide.

Embodiment 41. The compound, or a stereoisomer, tautomer, prodrug, or salt thereof, of embodiment 40, wherein the compound comprises the structure of Formula (IV-a):

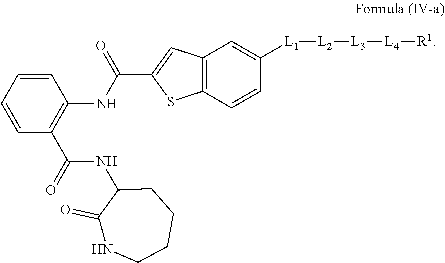

Embodiment 42. The compound, or a stereoisomer, tautomer, prodrug, or salt thereof, of embodiment 40, wherein the compound comprises the structure of Formula (IV-b):

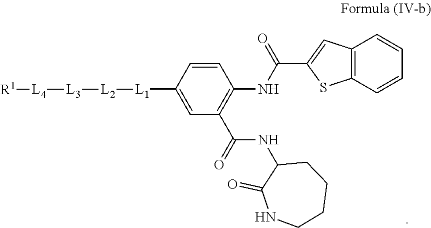

Embodiment 43. A compound, or a stereoisomer, tautomer, prodrug, or salt thereof, comprising the structure of Formula (VII):

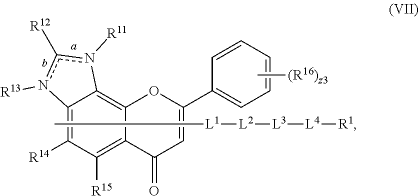

wherein:
each of L¹, L², L³, and L⁴ is independently a linker, bond, or absent;
R¹ is an oligonucleotide, a protecting group, a small molecule, a protein, an antibody, or a peptide;
R¹¹ and R₁₃ are each independently absent, hydrogen, or optionally substituted alkyl;
R¹², R¹⁴, and R¹⁵ are each independently hydrogen, optionally substituted alkyl, optionally substituted heteroalkyl, optionally substituted cycloalkyl, optionally substituted heterocycloalkyl, optionally substituted aryl, or optionally substituted heteroaryl;
R¹⁶ is hydrogen, halogen, CN, —N₃, —SOₙ₁₆R^{1A}, —SOᵥ₁₆NR^{16B}R^{16C}, —NHNR^{16B}R^{16C}, —ONR^{16B}R^{16C}, —NHC(O)NHNR^{16B}R^{16C}, —NHC(O)NR^{16B}R^{16C}, —N(O)ₘ₁₆, —NR^{16B}R^{16C}, —C(O)R^{16D}, —C(O)OR^{16D}, —C(O)NR^{16B}R^{16C}, —OR^{16A}, —NR^{16B}SO₂R^{16A}, —NR^{16B}C(O)R^{16D}; —NR^{16B}C(O)OR^{16D}, —NR^{16B}OR^{16D}, optionally substituted alkyl, optionally substituted heteroalkyl, optionally substituted cycloalkyl, optionally substituted heterocycloalkyl, optionally substituted aryl, or optionally substituted heteroaryl;
[symbol] and [symbol] are each independently a single bond or a double bond, wherein if [symbol] is a single bond, then [symbol] is a double bond and R¹³ is absent; and further wherein if [symbol] is a single bond, then [symbol] is a double bond and R¹¹ is absent;
R^{16A}, R^{16B}, R^{16C}, R^{16D} are each independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{16B} and R^{16C} substituents bonded to the same nitrogen atom may optionally be joined to form a substituted or unsubstituted heterocycloalkyl or substituted or unsubstituted heteroaryl;
z3 is 0, 1, 2, 3, 4, or 5;
n16 is 0, 1, 2, 3, or 4; and
v16 and m16 are each independently 1 or 2.

Embodiment 44. The compound, or a stereoisomer, tautomer, prodrug, or salt thereof, of embodiment 43, wherein the compound comprises the structure of Formula (VII-a), Formula (VII-b), or a mixture thereof:

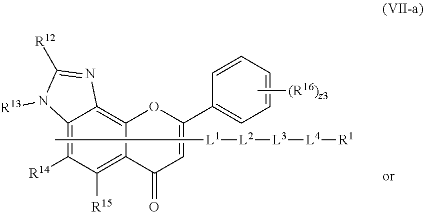

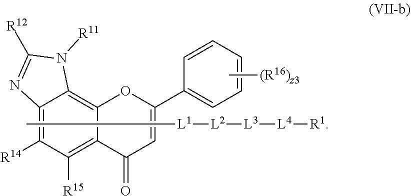

Embodiment 45. The compound, or a stereoisomer, tautomer, prodrug, or salt thereof, of embodiment 44, wherein the compound comprises the structure of Formula (VII-c):

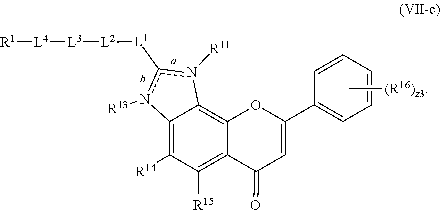

Embodiment 46. The compound, or a stereoisomer, tautomer, prodrug, or salt thereof, of embodiment 44 or 45, wherein the compound comprises the structure of Formula (VII-c-1), Formula (VII-c-2), or a mixture thereof:

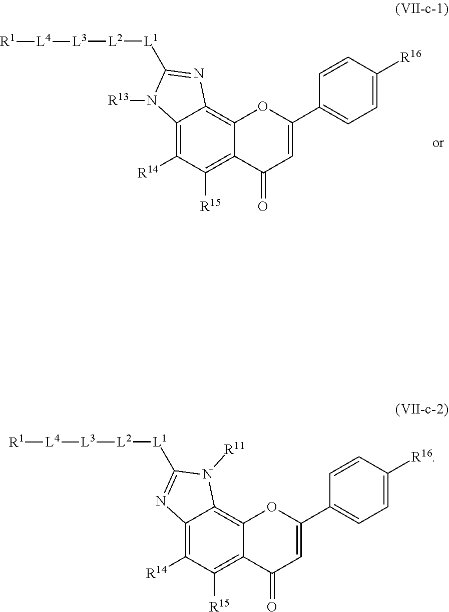

Embodiment 47. The compound, or a stereoisomer, tautomer, prodrug, or salt thereof, of embodiment 46, wherein R¹⁶ is hydrogen, optionally substituted alkyl, or optionally substituted heteroalkyl.

Embodiment 48. The compound, or a stereoisomer, tautomer, prodrug, or salt thereof, of embodiment 46 or 47, wherein:
R¹⁶ is —NR^{16B}R^{16C}; and
R^{16B} and R^{16C} are each independently hydrogen or optionally substituted alkyl.

Embodiment 49. The compound, or a stereoisomer, tautomer, prodrug, or salt thereof, of embodiment 48, wherein:
R^{16B} is hydrogen; and
R^{16C} is C₁-C₃ alkyl.

Embodiment 50. The compound, or a stereoisomer, tautomer, prodrug, or salt thereof, of embodiment 49, wherein R^{16C} is —CH₃.

Embodiment 51. The compound, or a stereoisomer, tautomer, prodrug, or salt thereof, of embodiment 43, wherein the compound comprises the structure of Formula (VII-d):

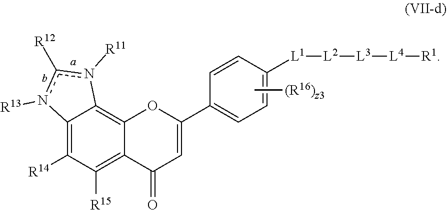

Embodiment 52. The compound, or a stereoisomer, tautomer, prodrug, or salt thereof, of embodiment 43 or 51, wherein the compound comprises the structure of Formula (VII-d-1), Formula (VII-d-2), or a mixture thereof:

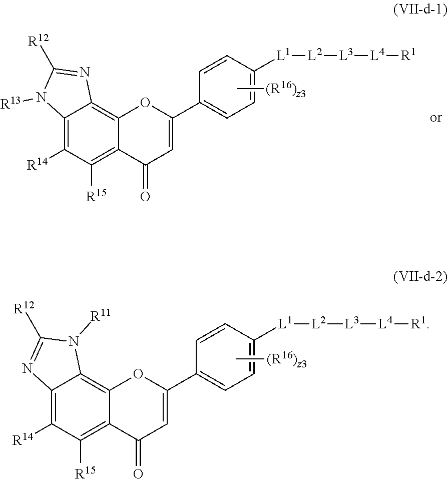

Embodiment 53. The compound, or a stereoisomer, tautomer, prodrug, or salt thereof, of embodiment 52, wherein R¹² is hydrogen or optionally substituted alkyl.

Embodiment 54. The compound, or a stereoisomer, tautomer, prodrug, or salt thereof, of embodiment 53, wherein R¹² is hydrogen or C₁-C₃ alkyl.

Embodiment 55. The compound, or a stereoisomer, tautomer, prodrug, or salt thereof, of embodiment 54, wherein R¹² is hydrogen or —CH₃.

Embodiment 56. A compound, or a stereoisomer, tautomer, prodrug, or salt thereof, wherein the compound comprises the structure of Formula (II″):

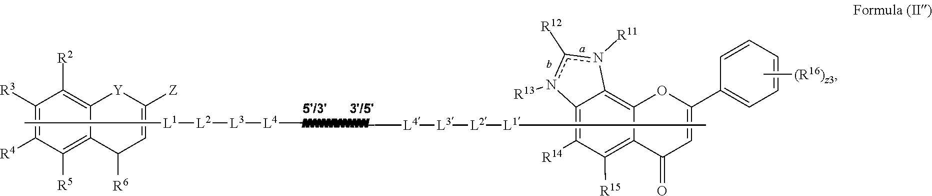

wherein:
[symbol] is an oligonucleotide;
each of L¹, L², L³, L⁴, L^{1′}, L^{2′}, L^{3′}, and L^{4′} is independently a linker, bond, or absent;
R² is hydrogen, —OR⁷, —SR⁸, or —NR⁹R¹⁰;
R³ is hydrogen, —OR³¹, —SR³², or —NR³³R³⁴;
R⁴ is hydrogen, —OR³⁵, —SR³⁶, or —NR³⁷R³⁸;
R⁵ is hydrogen, —OR³⁹, —SR⁴⁰, or —NR⁴¹R⁴²;
R⁶ is hydrogen, —OH, optionally substituted —O-alkyl, optionally substituted —OAc, —NH₂,
optionally substituted —NHAc, —SH, or ═O;
R⁷, R⁸, R⁹, R¹⁰, R³¹, R³², R³³, R³⁴, R³⁵, R³⁶, R³⁷, R³⁸, R³⁹, R⁴⁰, R⁴¹, and R⁴² are each independently hydrogen, optionally substituted alkyl, optionally substituted heteroalkyl, optionally substituted cycloalkyl, optionally substituted heterocycloalkyl, optionally substituted aryl, optionally substituted heteroaryl;
Y is CH₂, NH, S, or O;
Z is optionally substituted aryl or optionally substituted heteroaryl;
R¹¹ and R₁₃ are each independently absent, hydrogen, or optionally substituted alkyl;
R¹², R¹⁴, R¹⁵, and R¹⁶ are each independently hydrogen, optionally substituted alkyl, optionally substituted heteroalkyl, optionally substituted cycloalkyl, optionally substituted heterocycloalkyl, optionally substituted aryl, optionally substituted heteroaryl;
[symbol] and [symbol] are each independently a single bond or a double bond, wherein if [symbol] is a single bond, then [symbol] is a double bond and R¹³ is absent; and further wherein if [symbol] is a single bond, then [symbol] is a double bond and R¹¹ is absent; and
z3 is 0, 1, 2, 3, 4, or 5.

Embodiment 57. A compound, or a stereoisomer, tautomer, prodrug, or salt thereof, wherein the compound comprises the structure of Formula (II′″):

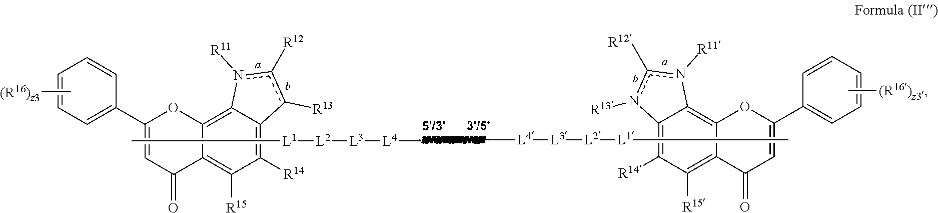

wherein:
[symbol] is an oligonucleotide;
each of L¹, L², L³, L⁴ or L^{1′}, L^{2′}, L^{3′}, and L^{4′} is independently a linker, bond, or absent;
R¹¹, R^{11′}, R¹³, and R^{13′} are each independently absent, hydrogen, or optionally substituted alkyl;
R¹², R^{12′}, R¹⁴, R^{14′}, R¹⁵, R^{15′}, R¹⁶, and R^{16′} are each independently hydrogen, optionally substituted alkyl, optionally substituted heteroalkyl, optionally substituted cycloalkyl, optionally substituted heterocycloalkyl, optionally substituted aryl, optionally substituted heteroaryl;
z3 and z3′ are each independently 0, 1, 2, 3, or 5;
[symbol] and [symbol] are each independently a single bond or a double bond, wherein if [symbol] is a single bond, then [symbol] is a double bond and R¹³ is absent; and further wherein if [symbol] is a single bond, then [symbol] is a double bond and R¹¹ is absent; and
[symbol] and [symbol] are each independently a single bond or a double bond, wherein if [symbol] is a single bond, then [symbol] is a double bond and R^{13′} is absent; and further wherein if [symbol] is a single bond, then [symbol] is a double bond and R^{11′} is absent.

Embodiment 58. A compound, or a stereoisomer, tautomer, prodrug, or salt thereof, comprising the structure of Formula (VIII):

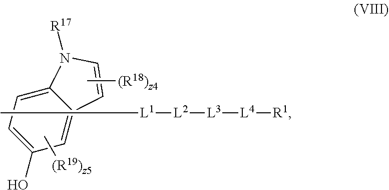

wherein:
each of L¹, L², L³, and L⁴ is independently a linker, bond, or absent;
R¹⁷, R¹⁸, and R¹⁹ are each independently hydrogen, optionally substituted alkyl, optionally substituted heteroalkyl, optionally substituted cycloalkyl, optionally substituted heterocycloalkyl, optionally substituted aryl, or optionally substituted heteroaryl;
z4 is 0, 1, or 2; and
z5 is 0, 1, 2, or 3.

Embodiment 59. The compound, or a stereoisomer, tautomer, prodrug, or salt thereof, of embodiment 58, wherein the compound comprises the structure of Formula (VIII-a):

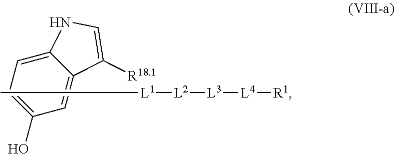

wherein:
R^{18.1} is hydrogen, optionally substituted alkyl or optionally substituted heteroalkyl.

Embodiment 60. The compound, or a stereoisomer, tautomer, prodrug, or salt thereof, of embodiment 58 or 59, wherein the compound comprises the structure of Formula (VIII-a-1):

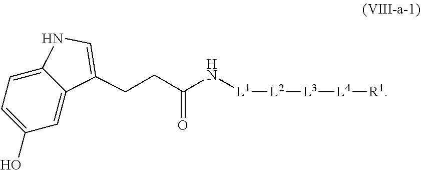

Embodiment 61. A compound, or a stereoisomer, tautomer, prodrug, or salt thereof, comprising the structure of Formula (IX):

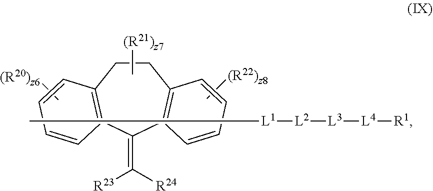

wherein:
each of L¹, L², L³, and L⁴ is independently a linker, bond, or absent;
R²⁰ is hydrogen, halogen, —CN, —N₃, —SOₙ₂₀R^{1A}, —SOᵥ₂₀NR^{20B}R^{20C}, —NHNR^{20B}R^{20C}, —ONR^{20B}R^{20C}, —NHC(O)NHNR^{20B}R^{20C}, —NHC(O)NR^{20B}R^{20C}, —N(O)ₘ₂₀, —NR^{20B}R^{20C}, —C(O)R^{20D}, —C(O)OR^{20D}, —C(O)NR^{20B}R^{20C}, —OR^{20A}, —NR^{20B}SO₂R^{20A}, —NR^{20B}C(O)R^{20D};
—NR^{20B}C(O)OR^{20D}, —NR^{20B}OR^{20D}, optionally substituted alkyl, optionally substituted heteroalkyl, optionally substituted cycloalkyl, optionally substituted heterocycloalkyl, optionally substituted aryl, or optionally substituted heteroaryl;
R²¹ is hydrogen, halogen, —CN, —N₃, —SOₙ₂₁R^{1A}, —SOᵥ₂₁NR^{21B}R^{21C}, —NHNR^{21B}R^{21C}, —ONR^{21B}R^{21C}, —NHC(O)NHNR^{21B}R^{21C}, —NHC(O)NR^{21B}R^{21C}, —N(O)ₘ₂₁, —NR^{21B}R^{21C}, —C(O)R^{21D}, —C(O)OR^{21D}, —C(O)NR^{21B}R^{21C}, —OR^{21A}, —NR^{21B}SO₂R^{21A}, —NR^{21B}C(O)R^{21D};
—NR^{21B}C(O)OR^{21D}, —NR^{21B}OR^{21D}, optionally substituted alkyl, optionally substituted heteroalkyl, optionally substituted cycloalkyl, optionally substituted heterocycloalkyl, optionally substituted aryl, or optionally substituted heteroaryl;
R²² and R²³ are each independently hydrogen, optionally substituted alkyl, optionally substituted heteroalkyl, optionally substituted cycloalkyl, optionally substituted heterocycloalkyl, optionally substituted aryl, or optionally substituted heteroaryl;
R²⁴ is hydrogen, halogen, —CN, —N₃, —SOₙ₂₄R^{24A}, —SOᵥ₂₄NR^{24B}R^{24C}, —NHNR^{24B}R^{24C}, —ONR^{24B}R^{24C}, —NHC(O)NHNR^{24B}R^{24C}, —NHC(O)NR^{24B}R^{24C}, —N(O)ₘ₂₄, —NR^{24B}R^{24C}, —C(O)R^{24D}, —C(O)OR^{24D}, —C(O)NR^{24B}R^{24C}, —OR^{24A}, —NR^{24B}SO₂R^{24A}, —NR^{24B}C(O)R^{24D};
—NR^{24B}C(O)OR^{24D}, —NR^{24B}OR^{24D}, optionally substituted alkyl, optionally substituted heteroalkyl, optionally substituted cycloalkyl, optionally substituted heterocycloalkyl, optionally substituted aryl, or optionally substituted heteroaryl;
R^{20A}, R^{20B}, R^{20C}, R^{20D}, R^{21A}, R^{21B}, R^{21C}, R^{21D}, R^{24A}, R^{24B}, R^{24C}, and R^{24D} are each independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{20B}, R^{20C}, R^{21B}, R^{21C}, R^{24B}, R^{24C}, R^{24B}, and R₂^{4C} substituents bonded to the same nitrogen atom may optionally be joined to form a substituted or unsubstituted heterocycloalkyl or substituted or unsubstituted heteroaryl;
n21, n22, n24, z6 and z8 are each independently 0, 1, 2, 3, or 4;
v20, v21, v24, m20, m21, and m24 are each independently 1 or 2; and
z7 is 0, 1, or 2.

Embodiment 62. The compound, or a stereoisomer, tautomer, prodrug, or salt thereof, of embodiment 61, wherein the compound comprises the structure of Formula (IX-a):

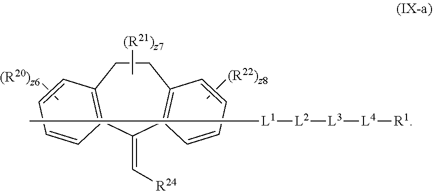

Embodiment 63. The compound, or a stereoisomer, tautomer, prodrug, or salt thereof, of embodiment 61 or 62, wherein the compound comprises the structure of Formula (IX-a-1):

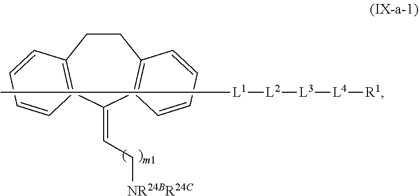

wherein:
R^{24B} and R^{24C} are each independently optionally substituted alkyl or optionally substituted heteroalkyl; and
m1 is 1, 2, or 3.

Embodiment 64. The compound, or a stereoisomer, tautomer, prodrug, or salt thereof, of any one of embodiments 61-63, wherein the compound comprises the structure of Formula (IX-a-2):

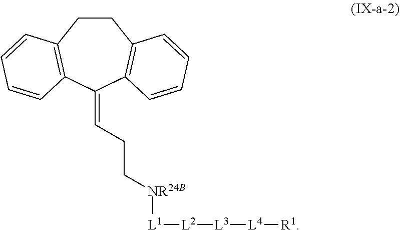

Embodiment 65. The compound, or a stereoisomer, tautomer, prodrug, or salt thereof, of embodiment 64, wherein R^{24B} is hydrogen or optionally substituted alkyl.

Embodiment 66. The compound, or a stereoisomer, tautomer, prodrug, or salt thereof, of embodiment 65, wherein R^{24B} is C₁-C₃ alkyl.

Embodiment 67. The compound, or a stereoisomer, tautomer, prodrug, or salt thereof, of embodiment 66, wherein R^{24B} is —CH₃.

Embodiment 68. A compound, or a stereoisomer, tautomer, prodrug, or salt thereof, comprising the structure of Formula (X):

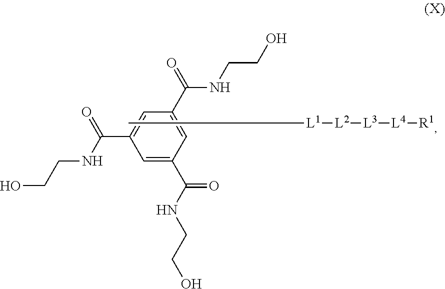

wherein each of L¹, L², L³, and L⁴ is independently a linker, bond, or absent; and
R¹ is an oligonucleotide, a protecting group, a small molecule, a protein, an antibody, or a peptide.

Embodiment 69. The compound, or a stereoisomer, tautomer, prodrug, or salt thereof, of embodiment 70, wherein the compound comprises the structure of Formula (X-a):

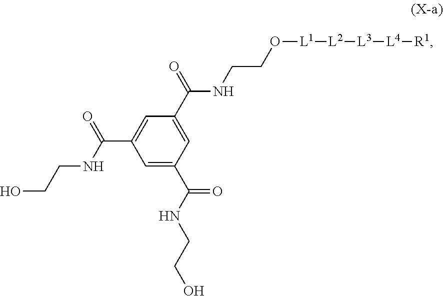

wherein each of L¹, L², L³, and L⁴ is independently a linker, bond, or absent; and
R¹ is an oligonucleotide, a protecting group, a small molecule, a protein, an antibody, or a peptide.

Embodiment 70. A compound, or a stereoisomer, tautomer, prodrug, or salt thereof, comprising the structure of Formula (XI):

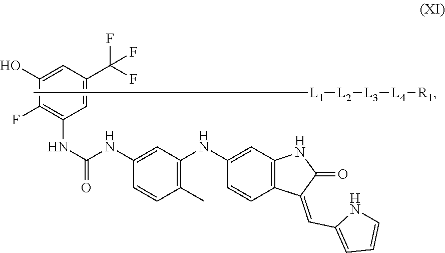

each of L¹, L², L³, and L⁴ is independently a linker, bond, or absent; and R¹ is an oligonucleotide, a protecting group, a small molecule, a protein, an antibody, or a peptide.

Embodiment 71. The compound, or a stereoisomer, tautomer, prodrug, or salt thereof, of embodiment 70, wherein the compound comprises the structure of Formula (XI-a):

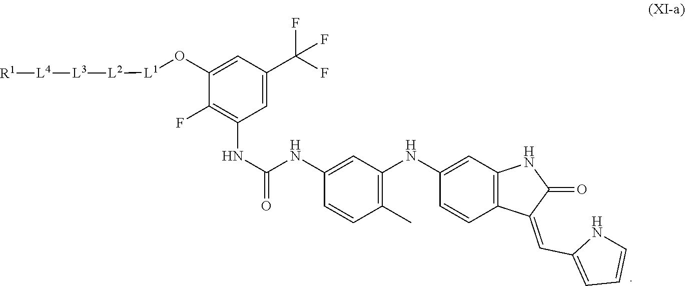

Embodiment 72. The compound, or a stereoisomer, tautomer, prodrug, or salt thereof, of any one of embodiments 1-71, wherein the TrkB ligand is a TrkB agonist, a TrkB partial agonist, or a TrkB antagonist.

Embodiment 73. The compound, or a stereoisomer, tautomer, prodrug, or salt thereof, of any one of embodiments 1-71, wherein the TrkB ligand is a TrkB selective ligand or a TrkB non-selective ligand.

Embodiment 74. The compound, or a stereoisomer, tautomer, prodrug, or salt thereof, of any one of embodiments 1-73, wherein each L¹, L², L³, L⁴, L^{1′}, L^{2′}, L^{3′}, and L^{4′} are each independently absent, a bond, an optionally substituted alkyl linker, an optionally substituted polyethylene glycol (PEG) linker, an optionally substituted heteroalkyl linker, or an optionally substituted heteroaryl linker.

Embodiment 75. The compound, or a stereoisomer, tautomer, prodrug, or salt thereof, of embodiment 74, wherein L¹ and L^{1′} are each independently an optionally substituted heteroaryl linker.

Embodiment 76. The compound, or a stereoisomer, tautomer, prodrug, or salt thereof, of embodiment 74, wherein L¹ and L^{1′} are each independently an optionally substituted unsaturated heteroaryl, an optionally substituted heteroaryl or an optionally substituted saturated or partially unsaturated heterocycloalkyl linker.

Embodiment 77. The compound, or a stereoisomer, tautomer, prodrug, or salt thereof, of embodiment 75 or 76, wherein L¹ and L^{1′} each independently comprise the structure

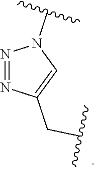 [SMILES: *Cc1cn(*)nn1]

Embodiment 78. The compound, or a stereoisomer, tautomer, prodrug, or salt thereof, of embodiment 74, wherein L¹ and L^{1′} are each independently an optionally substituted heteroalkyl linker.

Embodiment 79. The compound, or a stereoisomer, tautomer, prodrug, or salt thereof, of embodiment 78, wherein the optionally substituted heteroalkyl linker is an optionally substituted heteroalkyl or optionally substituted C₁-C₁₀ alkyl chain in which one or more carbon atoms are replaced with O, N, or S.

Embodiment 80. The compound, or a stereoisomer, tautomer, prodrug, or salt thereof, of embodiment 78 or 79, wherein L¹ and L^{1′} each independently comprise the structure

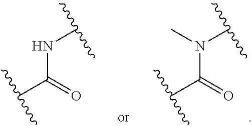

Embodiment 81. The compound, of salt thereof, of embodiment 78 or 79, wherein L¹ and L^{1′} each independently comprise the structure

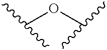 [SMILES: *O*]

### or —N(CH₃)—.

Embodiment 82. The compound, or a stereoisomer, tautomer, prodrug, or salt thereof, of any one of embodiments 74-81, wherein L² and L^{2′} are each independently an optionally substituted PEG linker.

Embodiment 83. The compound, or a stereoisomer, tautomer, prodrug, or salt thereof, of embodiment 82, wherein the PEG linker is five PEG units in length.

Embodiment 84. The compound, or a stereoisomer, tautomer, prodrug, or salt thereof, of embodiment 82, wherein the PEG linker is four PEG units in length.

Embodiment 85. The compound, of salt thereof, of embodiment 82, wherein the PEG linker is three PEG units in length.

Embodiment 86. The compound, or a stereoisomer, tautomer, prodrug, or salt thereof, of any one of embodiments 74-85, wherein L² and L^{2′} are each independently an optionally substituted alkyl linker.

Embodiment 87. The compound, or a stereoisomer, tautomer, prodrug, or salt thereof, of embodiment 86, wherein L² and L^{2′} are each independently an optionally substituted C₁₋₂₀ alkyl linker.

Embodiment 88. The compound, or a stereoisomer, tautomer, prodrug, or salt thereof, of embodiment 86 or 87, wherein L² and L^{2′} are each independently an optionally substituted C₈ alkyl linker.

Embodiment 89. The compound, or a stereoisomer, tautomer, prodrug, or salt thereof, of any one of embodiments 76-88, wherein L³ and L^{3′} are each independently is an optionally substituted heteroaryl linker.

Embodiment 90. The compound, or a stereoisomer, tautomer, prodrug, or salt thereof, of embodiment 89, wherein L³ and L^{3′} are each independently an optionally substituted partially unsaturated heteroaryl linker, an optionally substituted heteroaryl or an optionally substituted saturated or partially unsaturated heterocycloalkyl linker.

Embodiment 91. The compound, or a stereoisomer, tautomer, prodrug, or salt thereof, of embodiment 90, wherein L³ and L^{3′} each independently comprise the structure

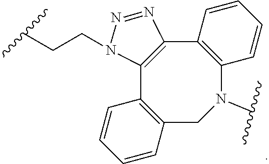 [SMILES: *CCn1nnc2c1-c1ccccc1CN(*)c1ccccc1-2]

Embodiment 92. The compound, or a stereoisomer, tautomer, prodrug, or salt thereof, of any one of embodiments 74-91, wherein L⁴ and L^{4′} are each independently an optionally substituted heteroalkyl linker.

Embodiment 93. The compound, or a stereoisomer, tautomer, prodrug, or salt thereof, of embodiment 92, wherein the heteroalkyl linker is substituted with one or more ═O substituents.

Embodiment 94. The compound, or a stereoisomer, tautomer, prodrug, or salt thereof, of embodiment 92 or 93, wherein the heteroalkyl linker comprises two substituents joined together to form an optionally substituted carbocyclyl ring.

Embodiment 95. The compound, or a stereoisomer, tautomer, prodrug, or salt thereof, of any one of embodiment 92-94, wherein L⁴ and L^{4′} each independently comprise the structure

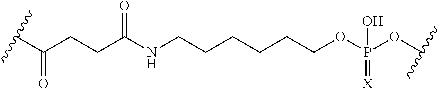

or a salt thereof,
wherein X is O or S.

Embodiment 96. The compound, or a stereoisomer, tautomer, prodrug, or salt thereof, of any one of embodiments 92-94, wherein L⁴ and L^{4′} each independently comprise the structure:

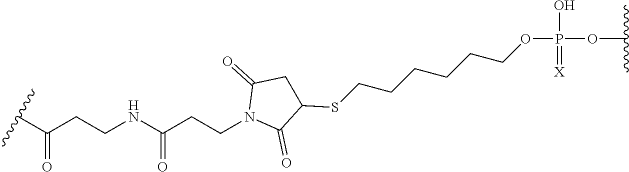

or a salt thereof,
wherein X is O or S.

Embodiment 97. The compound, or a stereoisomer, tautomer, prodrug, or salt thereof, of any one of embodiments 1-96, wherein L¹-L²-L³-L⁴; or L^{1′}-L^{2′}-L^{3′}-L^{4′} each independently comprise the structure:

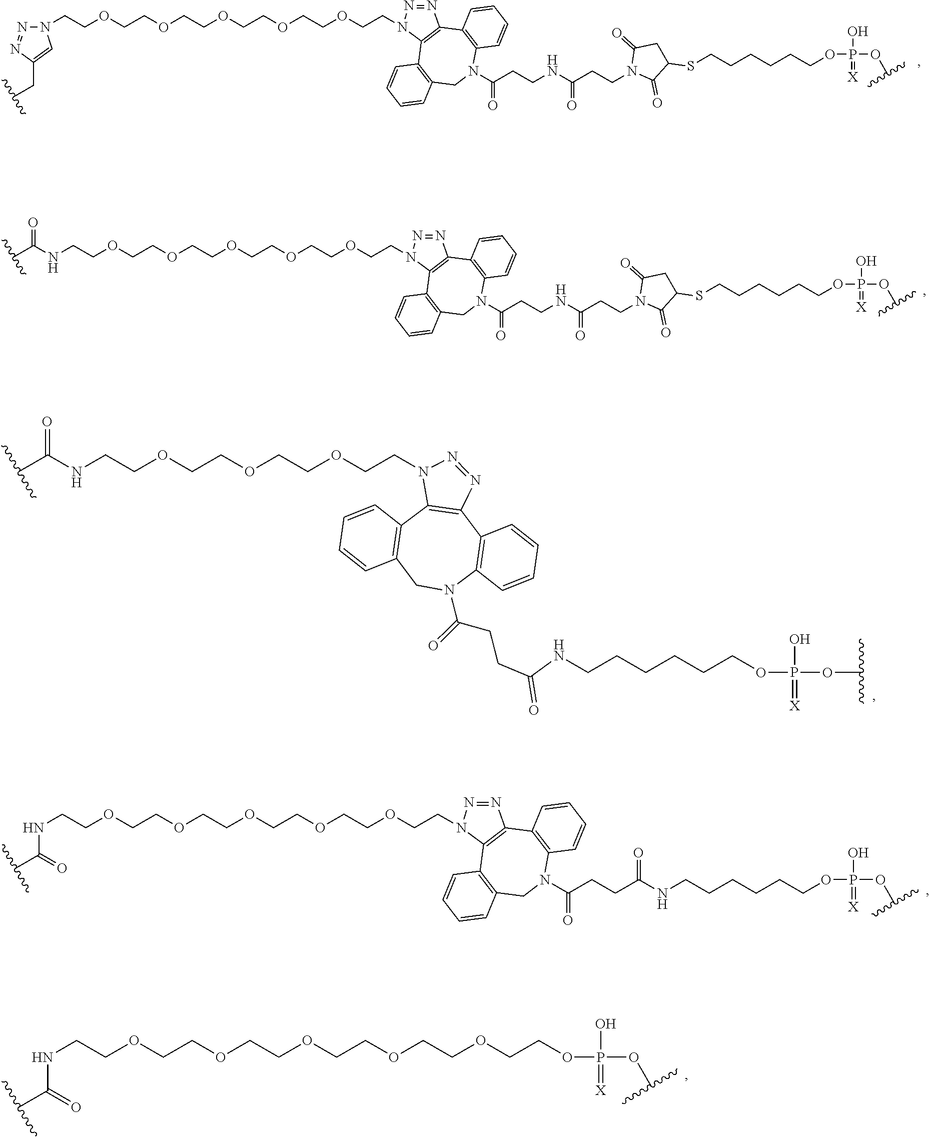

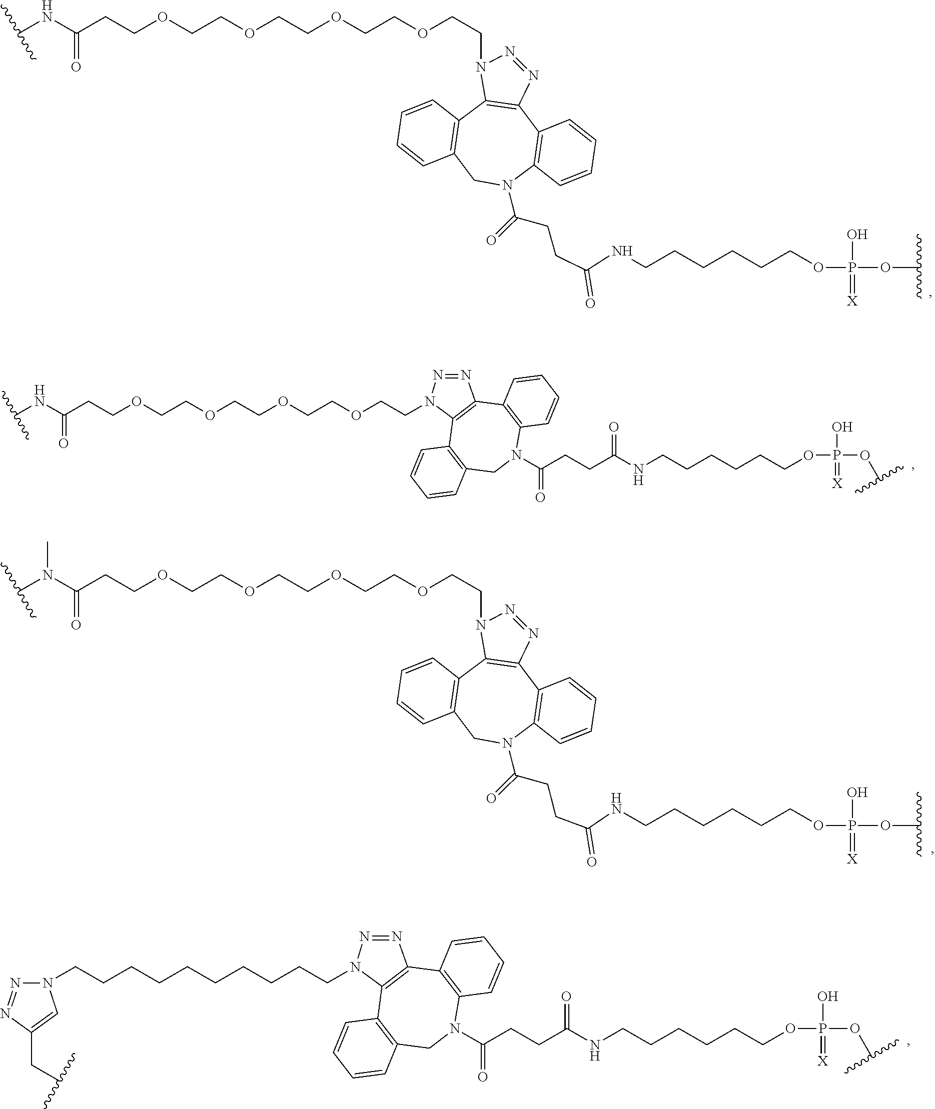

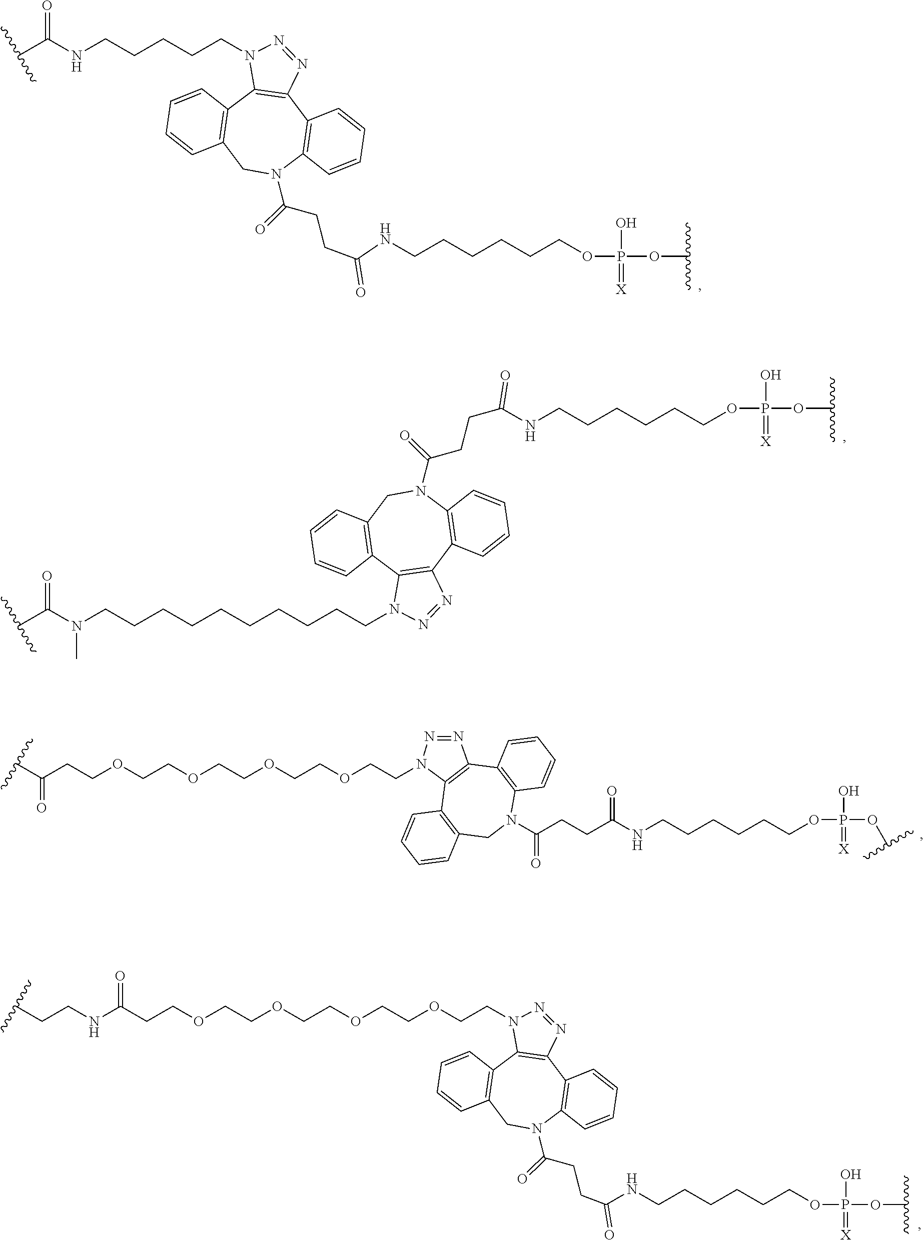

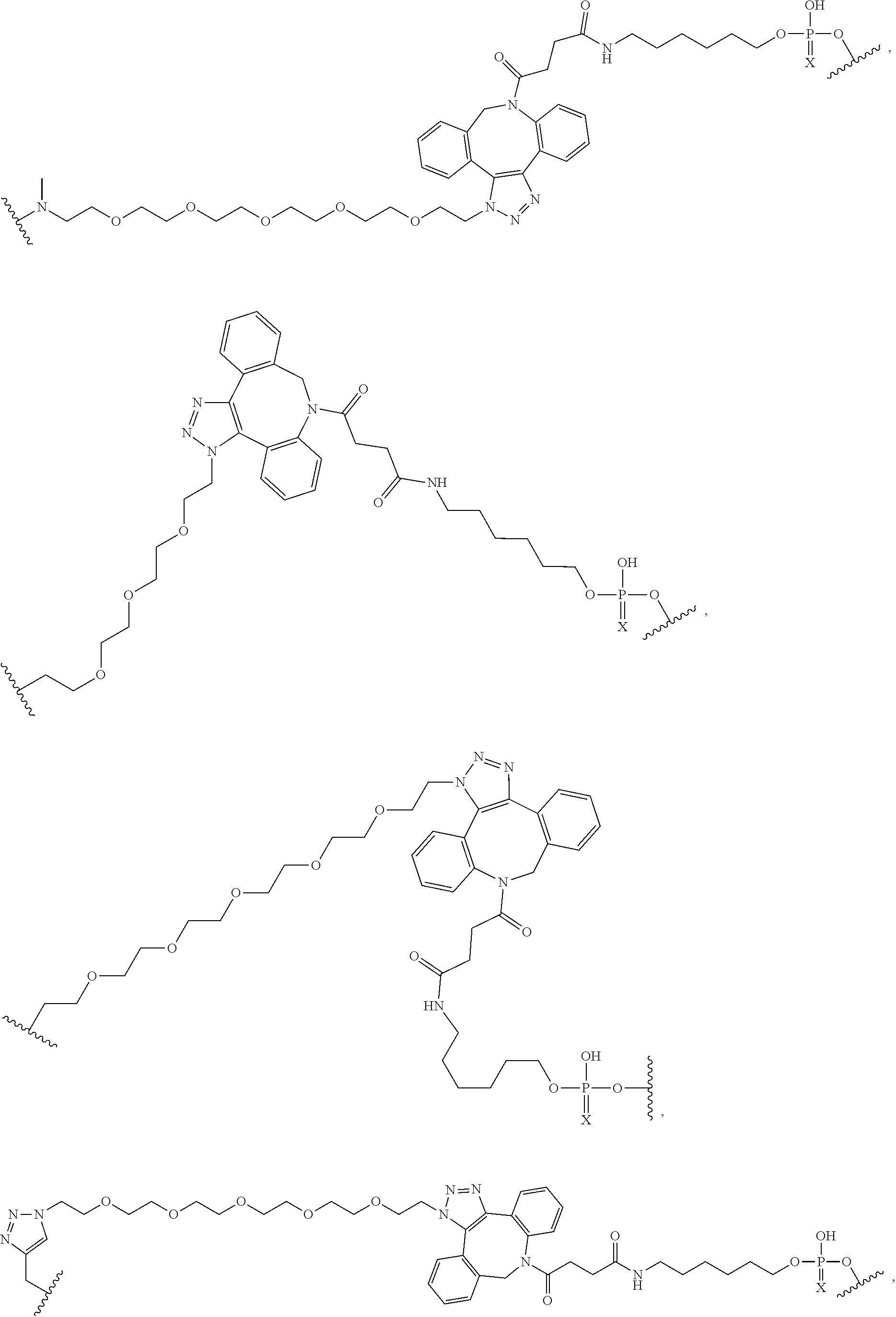

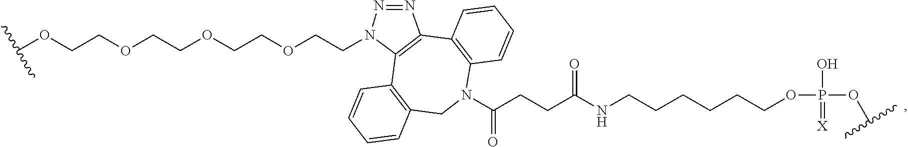

or a salt thereof, wherein X is O or S.

Embodiment 98. A compound, or a stereoisomer, tautomer, prodrug, or salt thereof, wherein the compound comprises the structure:

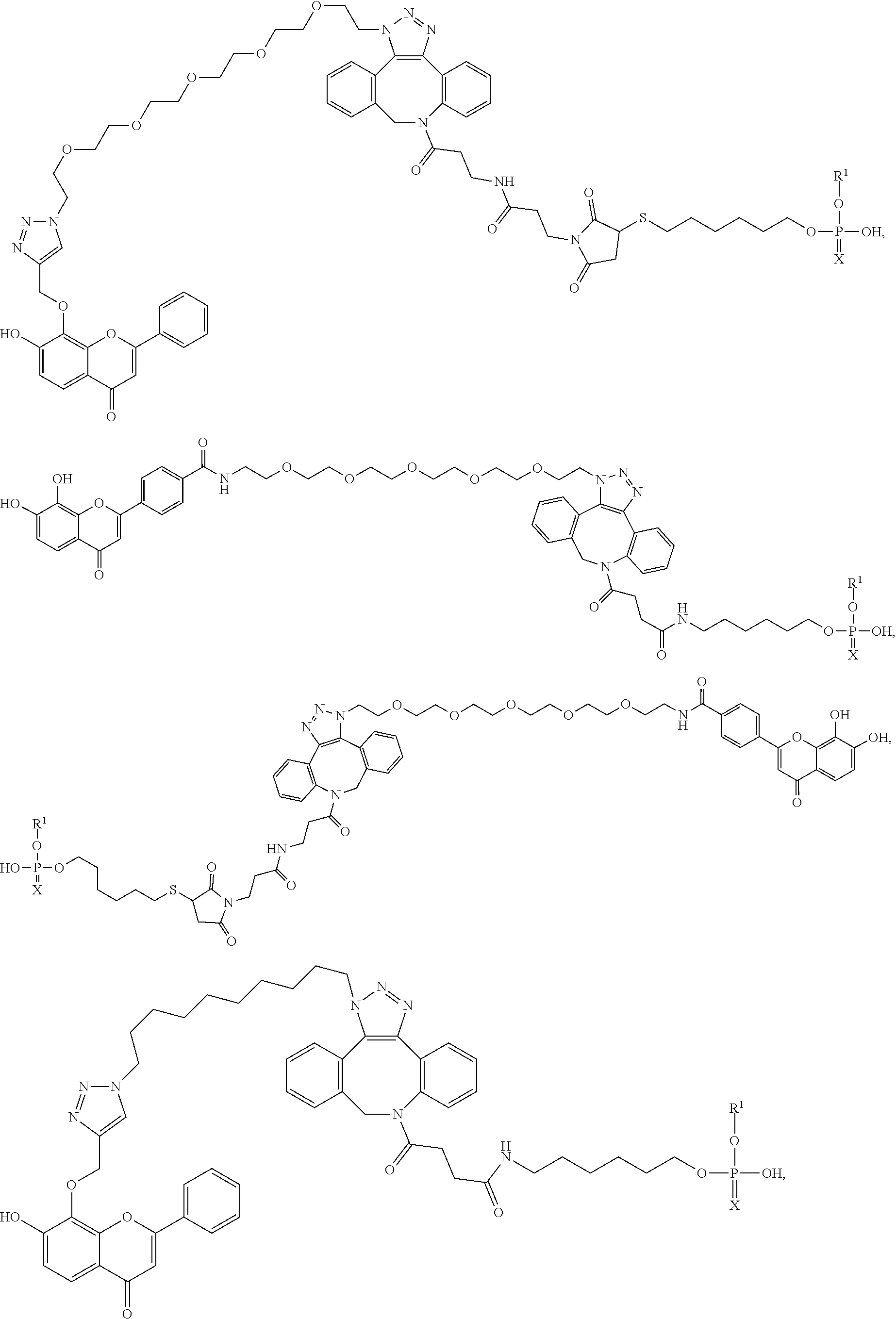

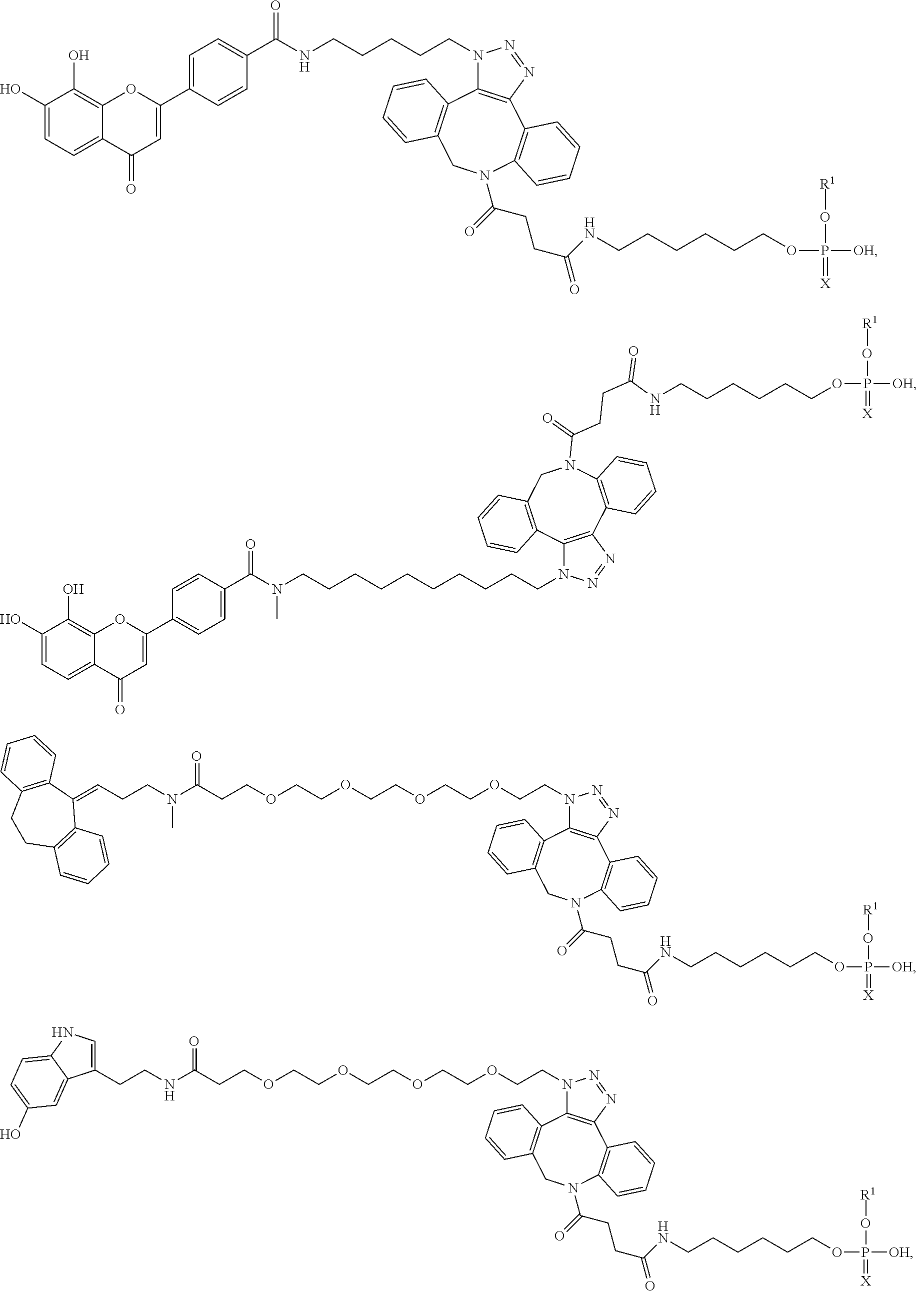

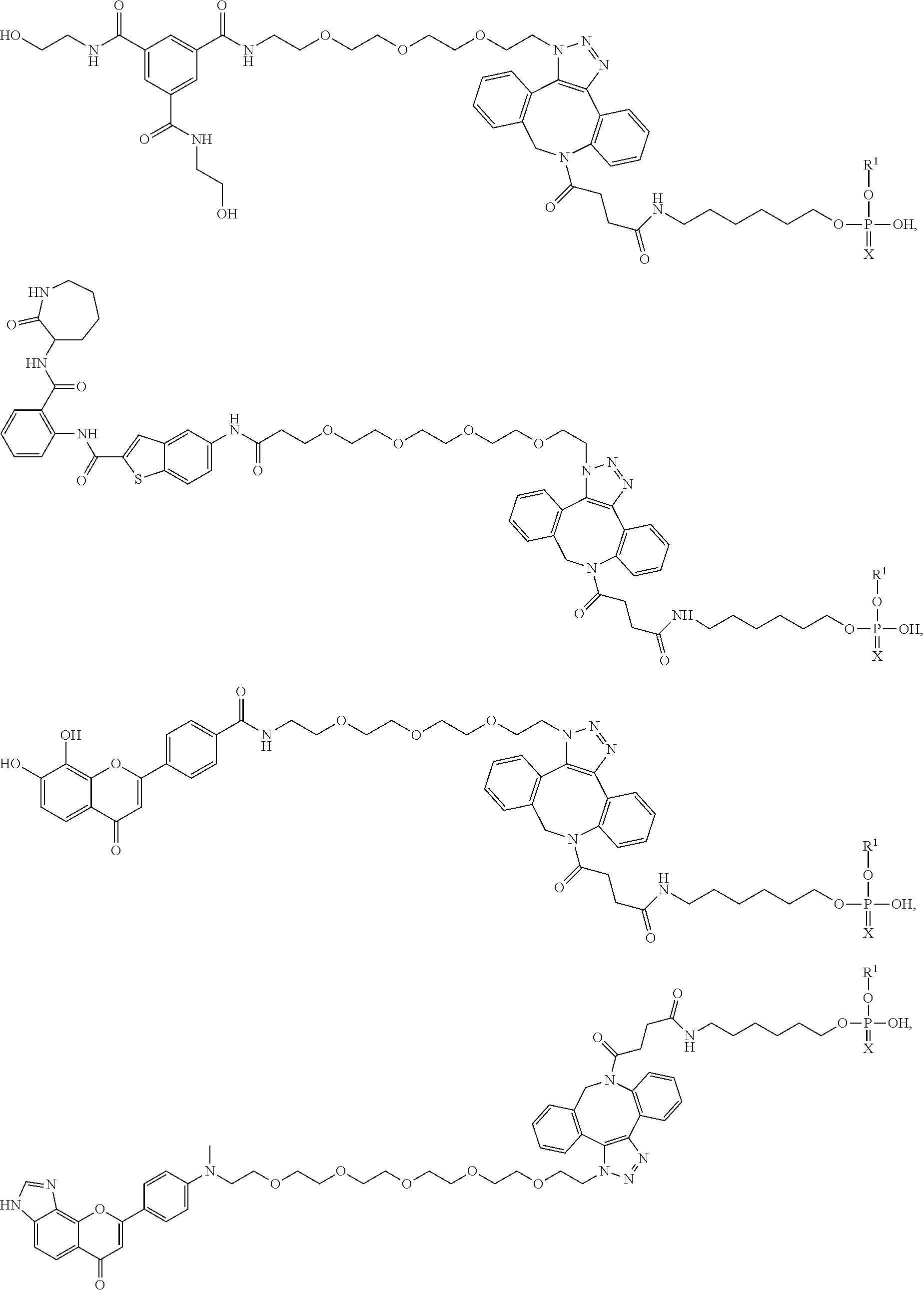

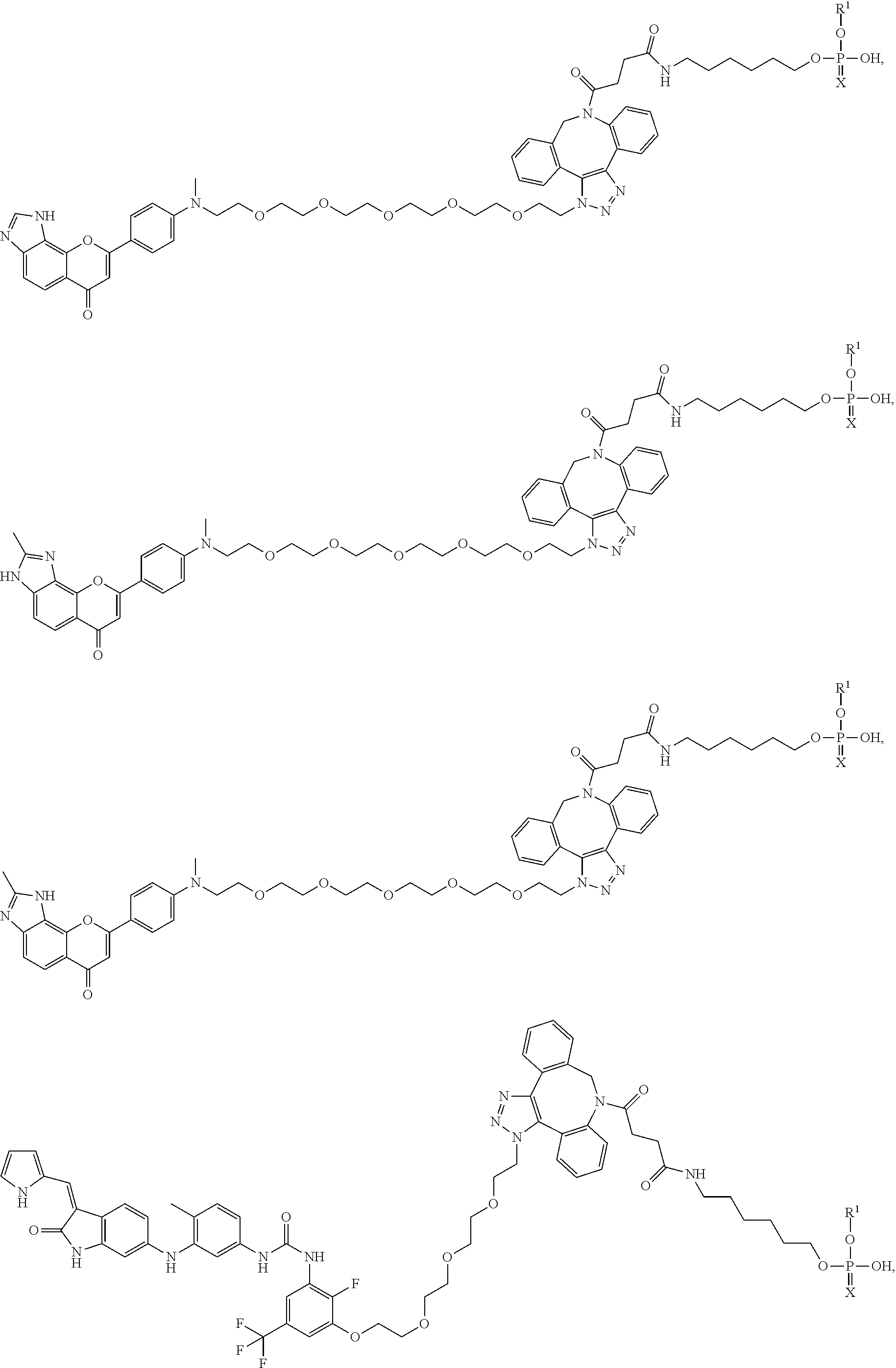

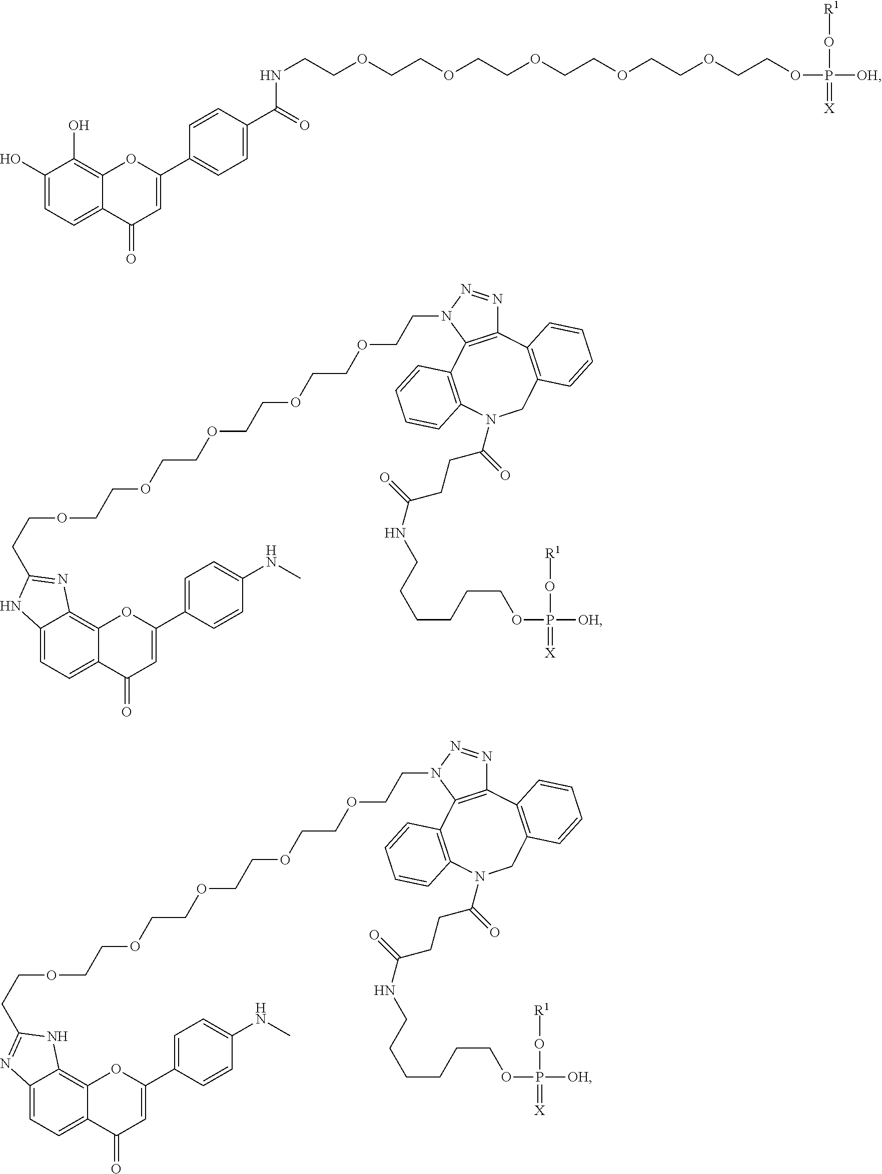

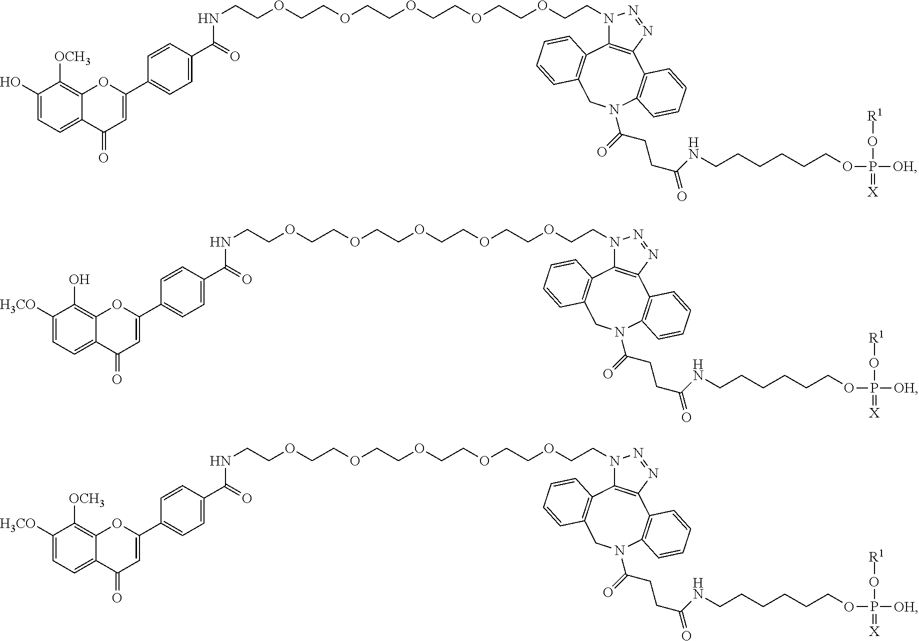

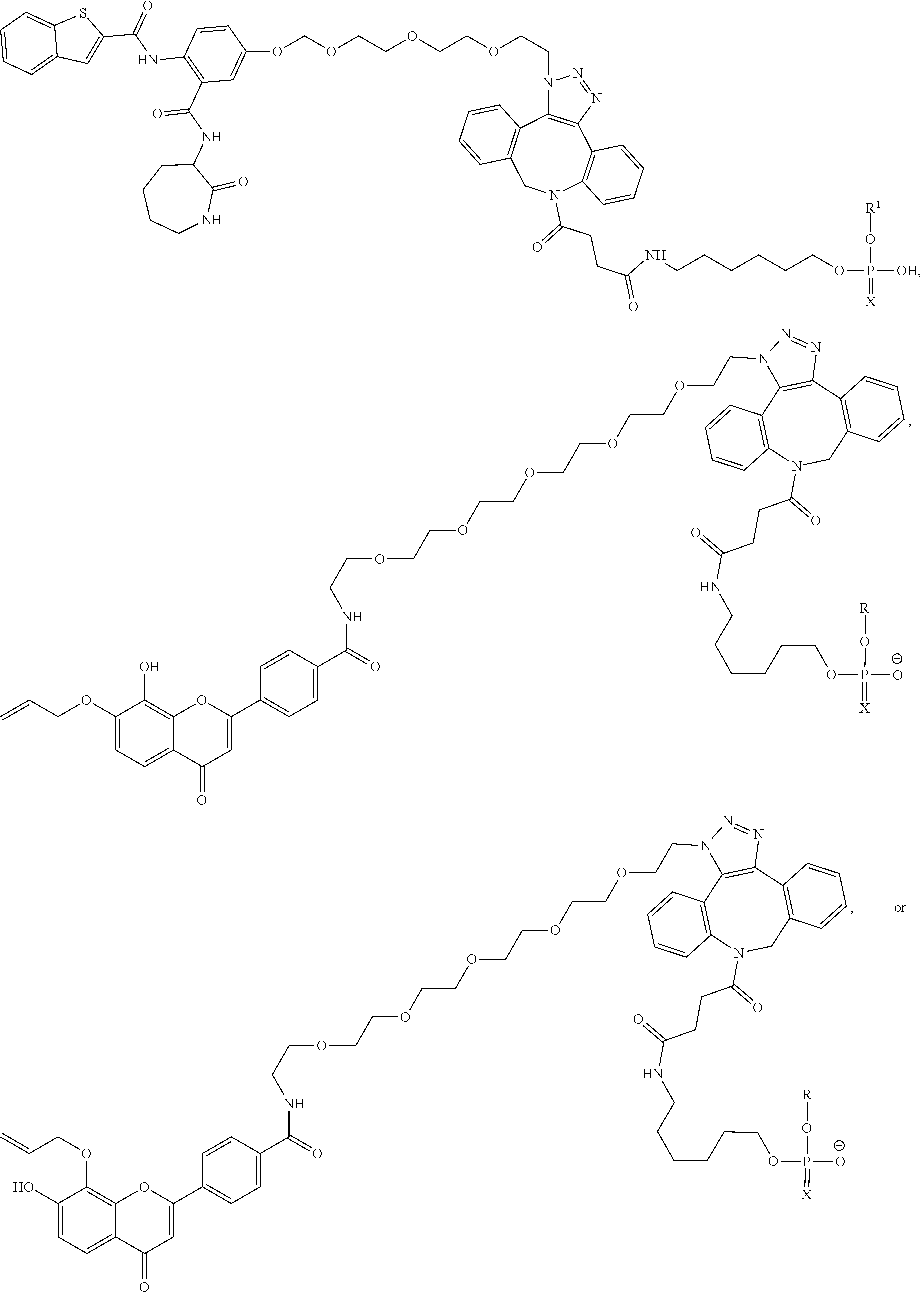

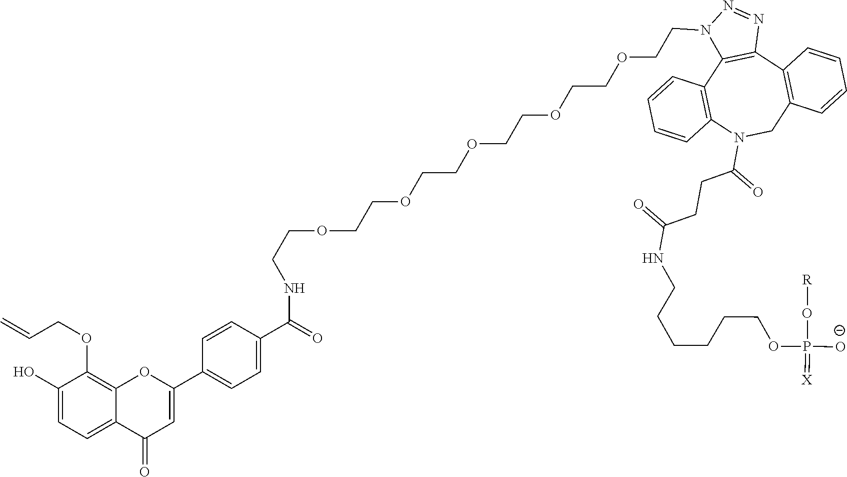

wherein:
R¹ is an oligonucleotide, a protecting group, a small molecule, a protein, an antibody, or a peptide; and
X is O or S.

Embodiment 99. A compound, or a stereoisomer, tautomer, prodrug, or salt thereof, wherein the compound comprises the structure:

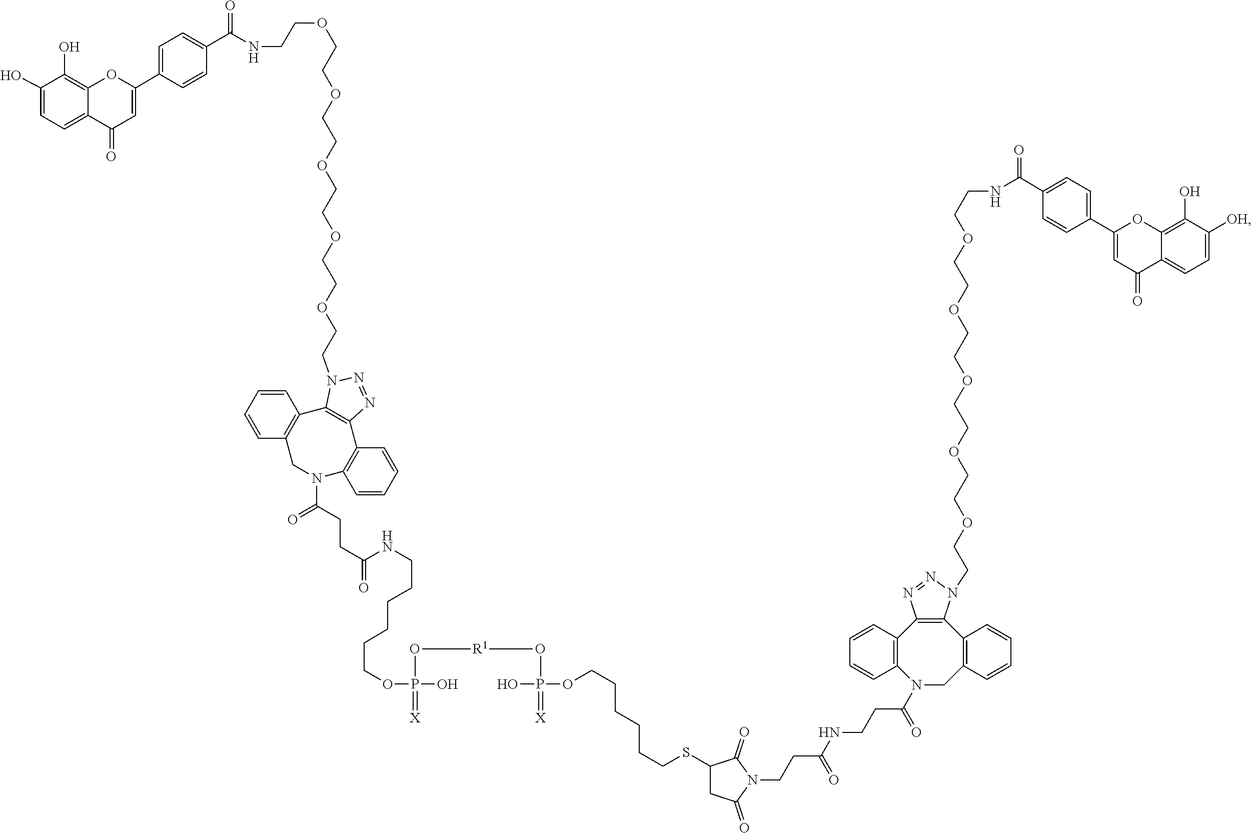

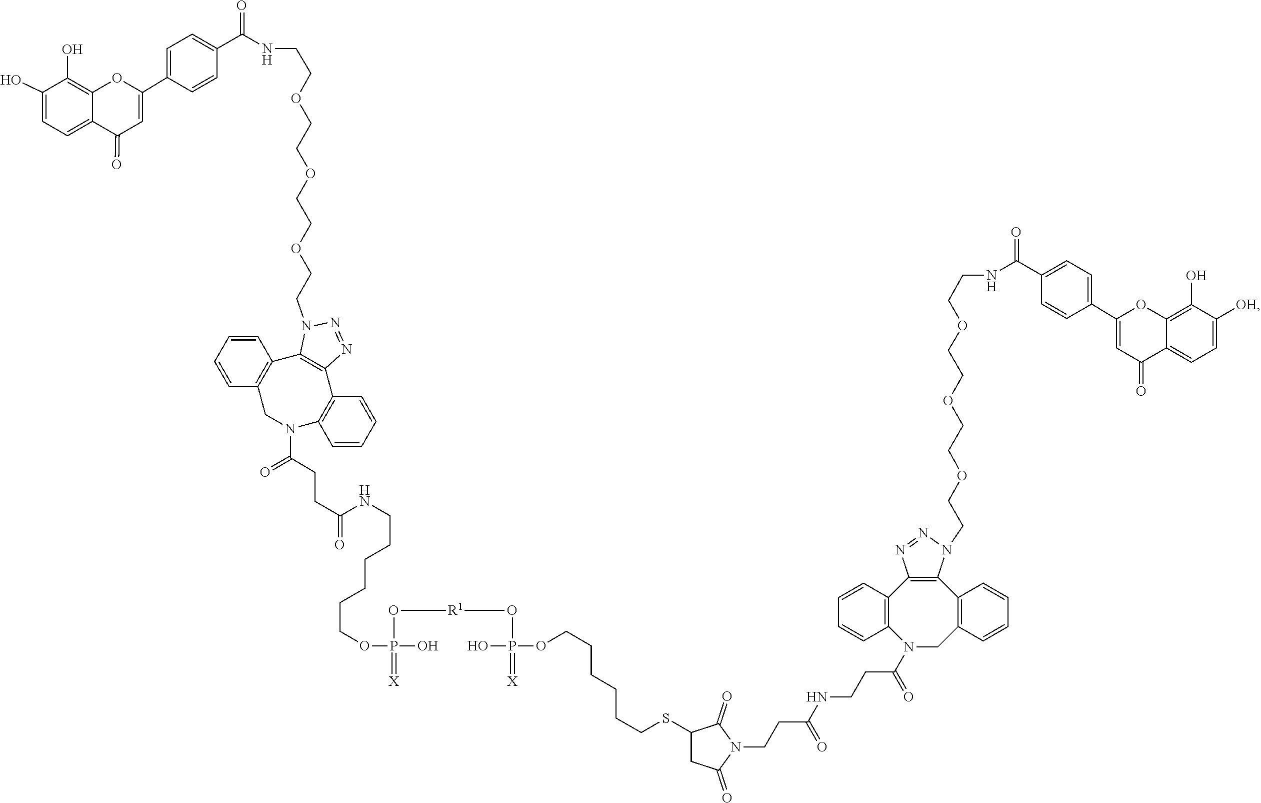

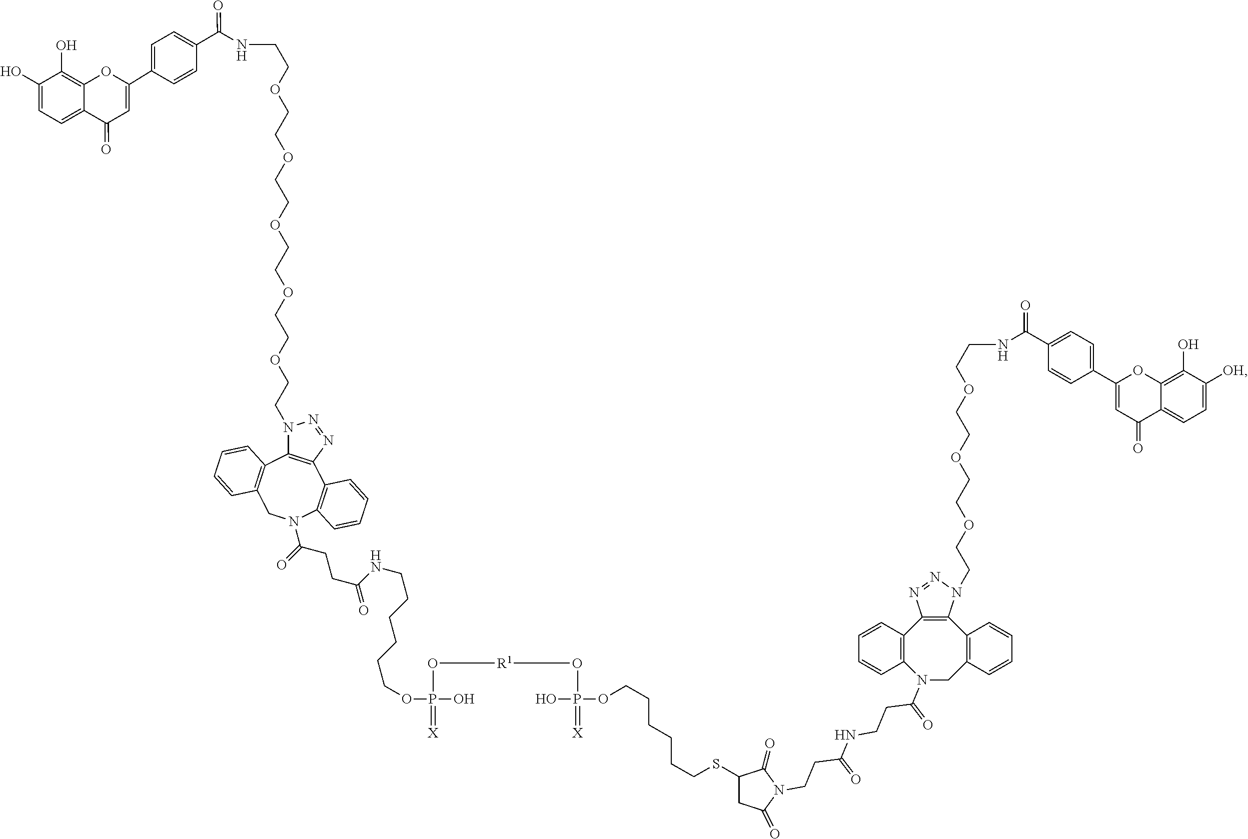

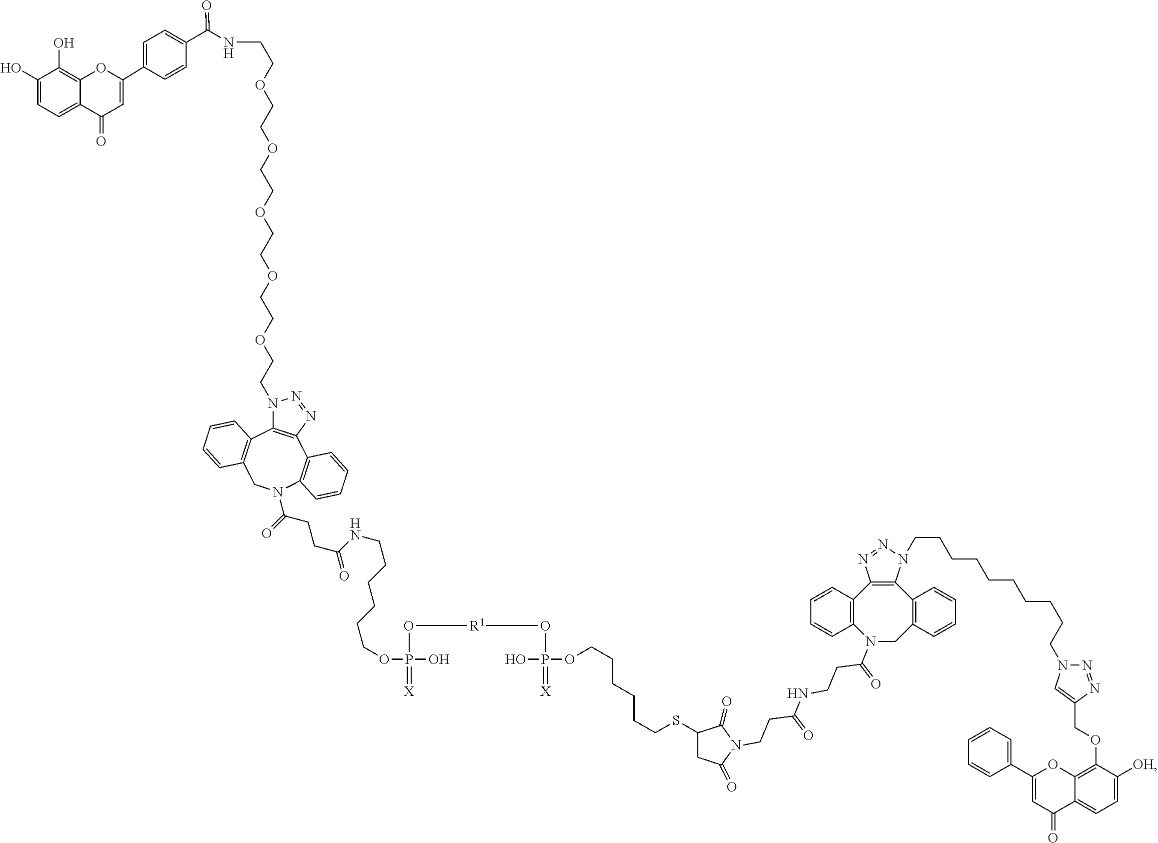

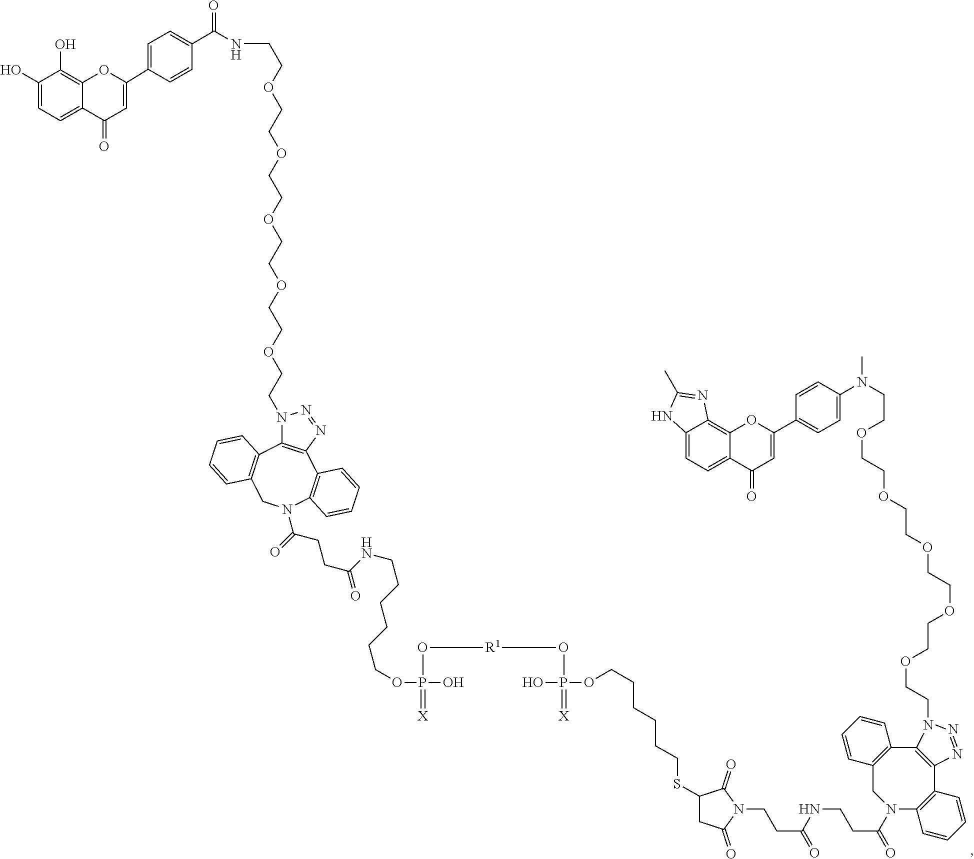

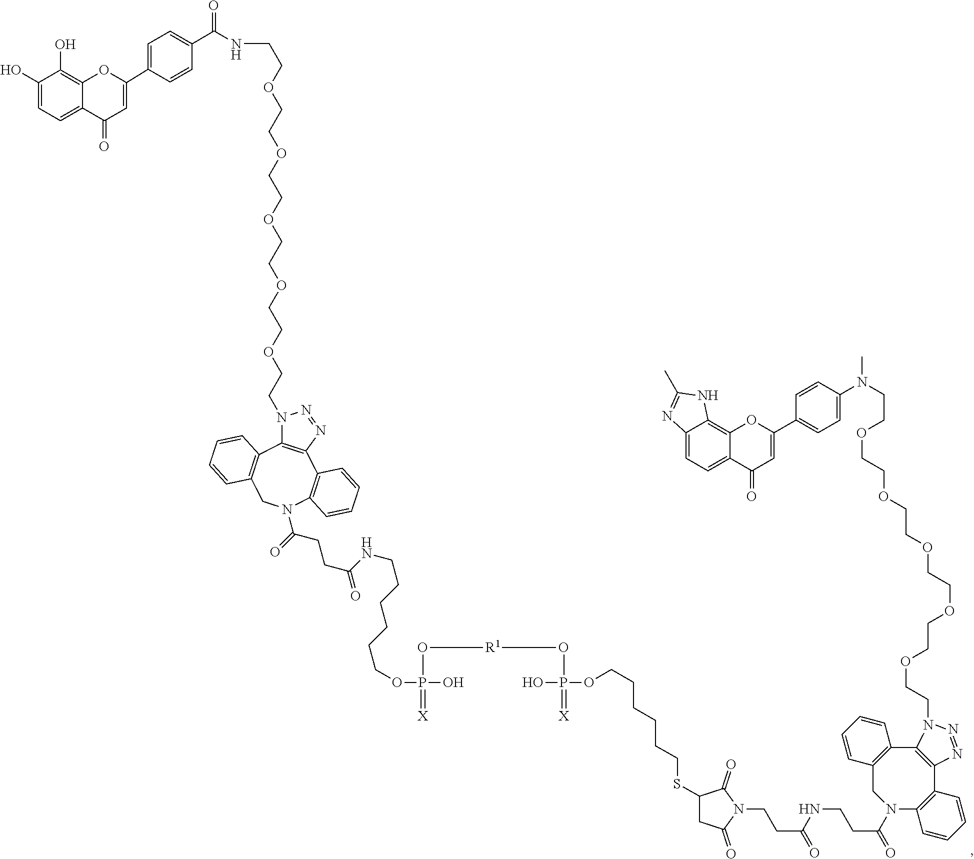

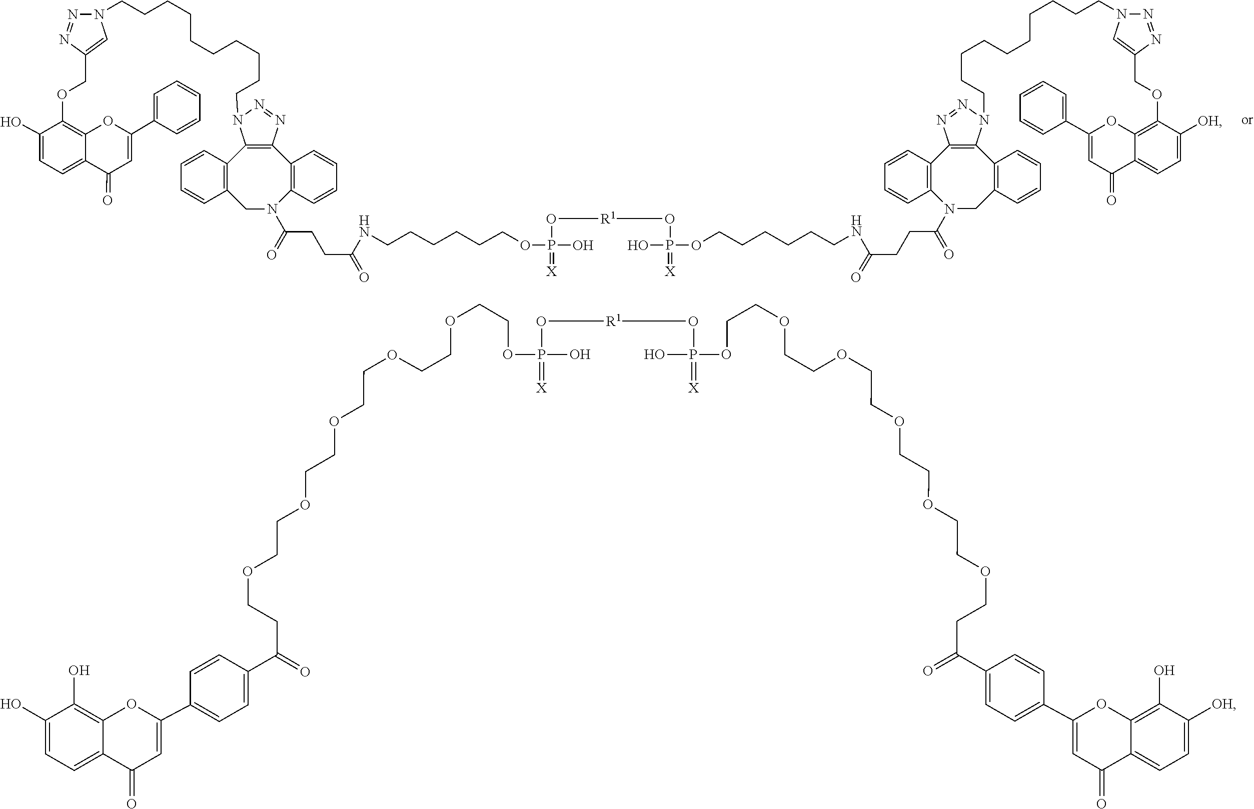

wherein:
R¹ is an oligonucleotide, a protecting group, a small molecule, a protein, an antibody, or a peptide; and
X is O or S.

Embodiment 100. A compound, or a stereoisomer, tautomer, prodrug, or salt thereof, wherein the compound comprises the structure:

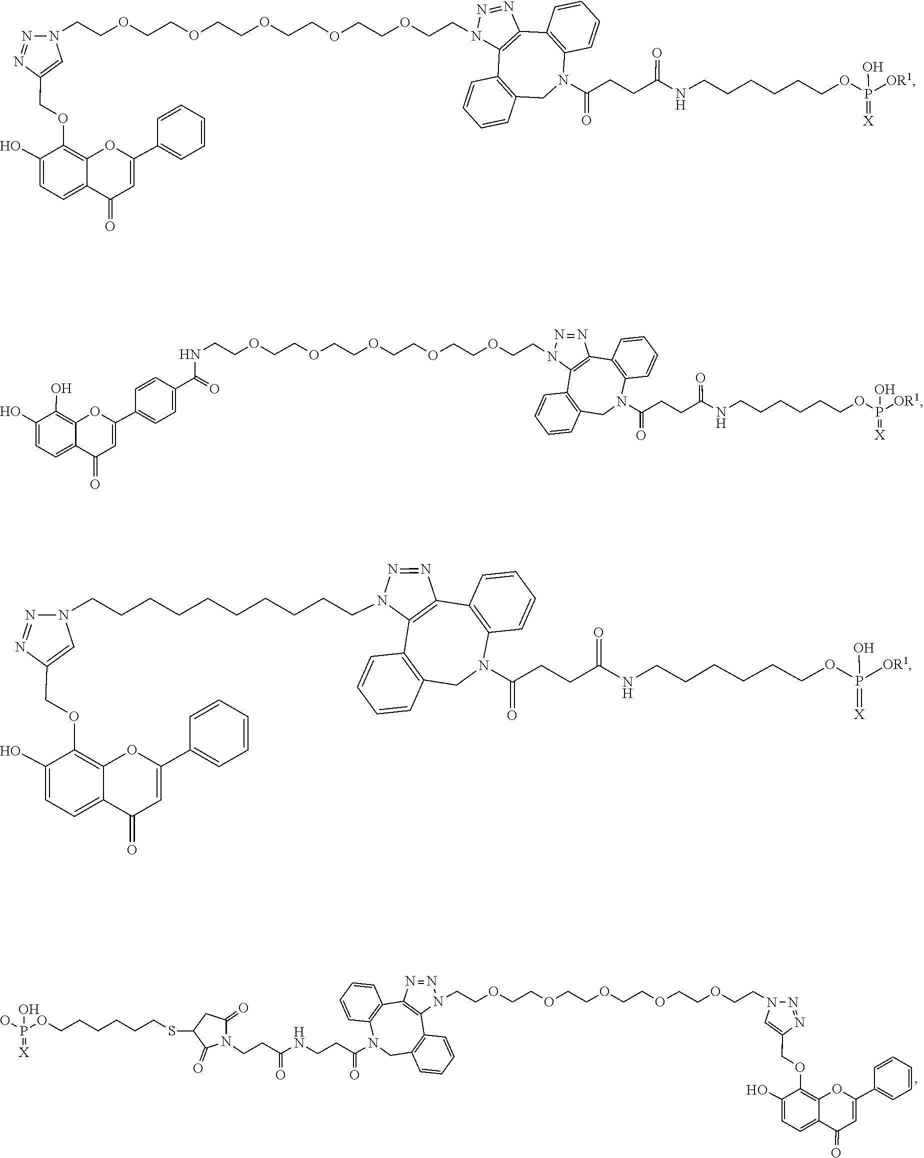

wherein:
R¹ is an oligonucleotide, a protecting group, a small molecule, a protein, an antibody, or a peptide; and
X is O or S.

Embodiment 101. A compound, or a stereoisomer, tautomer, prodrug, or salt thereof, wherein the compound comprises the structure:

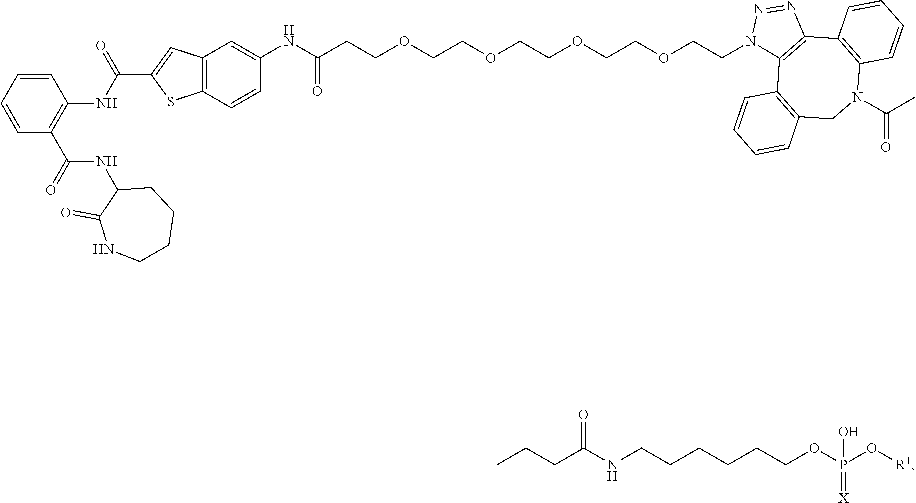

wherein:
R¹ is an oligonucleotide, a protecting group, a small molecule, a protein, an antibody, or a peptide; and
X is O or S.

Embodiment 102. The compound, or a stereoisomer, tautomer, prodrug, or salt thereof, of any one of embodiments 97-101, wherein X is O.

Embodiment 103. The compound, or a stereoisomer, tautomer, prodrug, or salt thereof, of any one of embodiments 97-101, wherein X is S.

Embodiment 104. The compound, or a stereoisomer, tautomer, prodrug, or salt thereof, of any one of embodiments 1-103, wherein R¹ comprises an oligonucleotide.

Embodiment 105. The compound, or a stereoisomer, tautomer, prodrug, or salt thereof, of embodiment 104, wherein the oligonucleotide is attached at its 5′ end.

Embodiment 106. The compound, or a stereoisomer, tautomer, prodrug, or salt thereof, of embodiment 104, wherein the oligonucleotide is attached at its 3′ end.

Embodiment 107. The compound, or a stereoisomer, tautomer, prodrug, or salt thereof, of embodiment 104, wherein the oligonucleotide is attached at an internal position on the oligonucleotide.

Embodiment 108. The compound, or a stereoisomer, tautomer, prodrug, or salt thereof, of embodiment 104, wherein the internal position is an internucleoside linkage.

Embodiment 109. The compound, or a stereoisomer, tautomer, prodrug, or salt thereof, of any one of embodiments 1-108, wherein R¹ comprises an oligonucleotide conjugated to one or more additional TrkB ligands.

Embodiment 110. The compound, or a stereoisomer, tautomer, prodrug, or salt thereof, of embodiment 109, wherein the oligonucleotide is conjugated to two, three, four, five, or more than five additional TrkB ligands.

Embodiment 111. The compound, or a stereoisomer, tautomer, prodrug, or salt thereof, of embodiment 109 or 110, wherein the additional TrkB ligands are conjugated to the oligonucleotide at the 5′ end of the oligonucleotide, the 3′ end of the oligonucleotide, the 5′- and 3′ ends of the oligonucleotide, one or more internal positions on the oligonucleotide, or any combination thereof.

Embodiment 112. The compound, or a stereoisomer, tautomer, prodrug, or salt thereof, of any one of embodiments 1-111, wherein the oligonucleotide is a modified oligonucleotide.

Embodiment 113. A compound, or a stereoisomer, tautomer, prodrug, or salt thereof, wherein the compound comprises the structure:

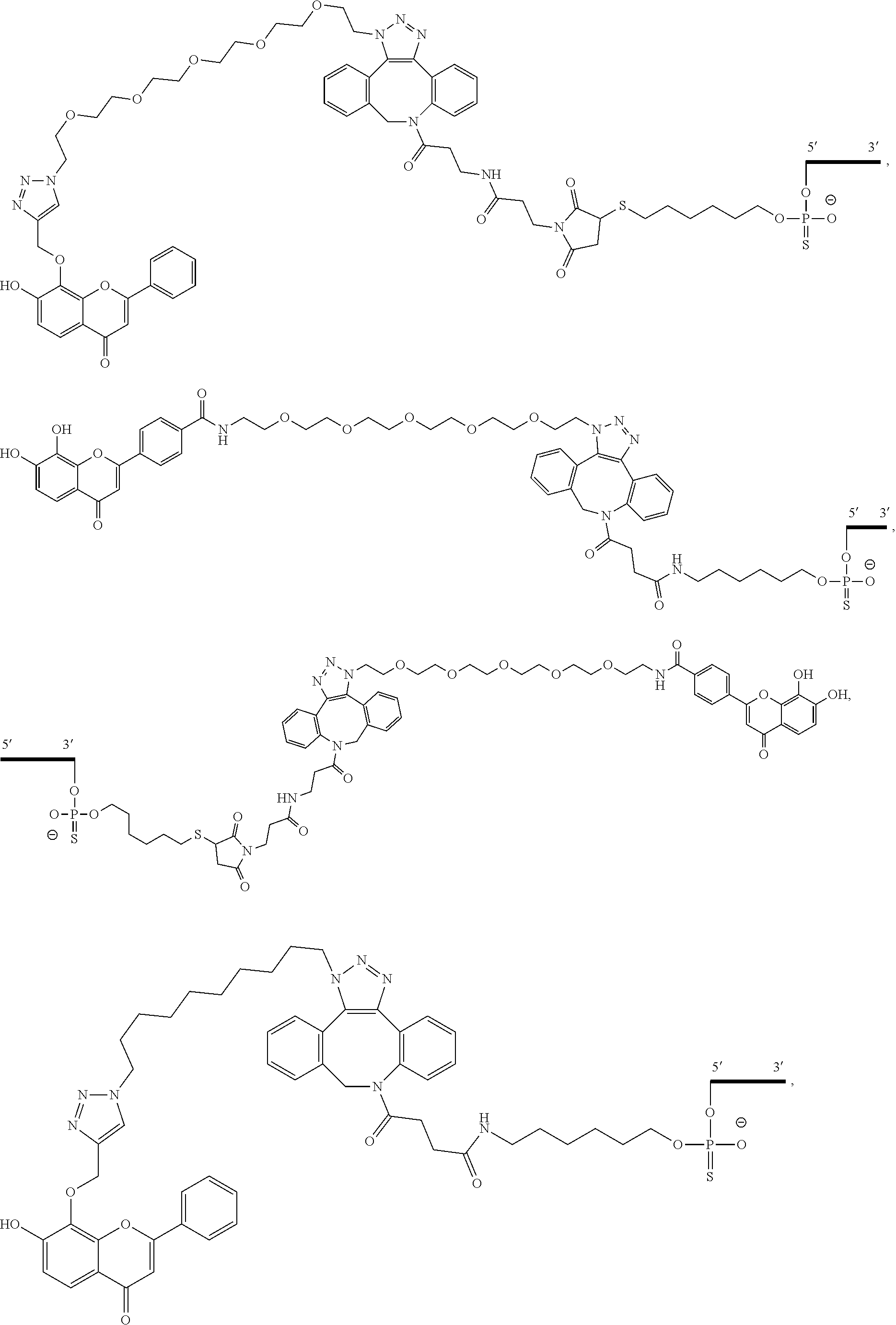

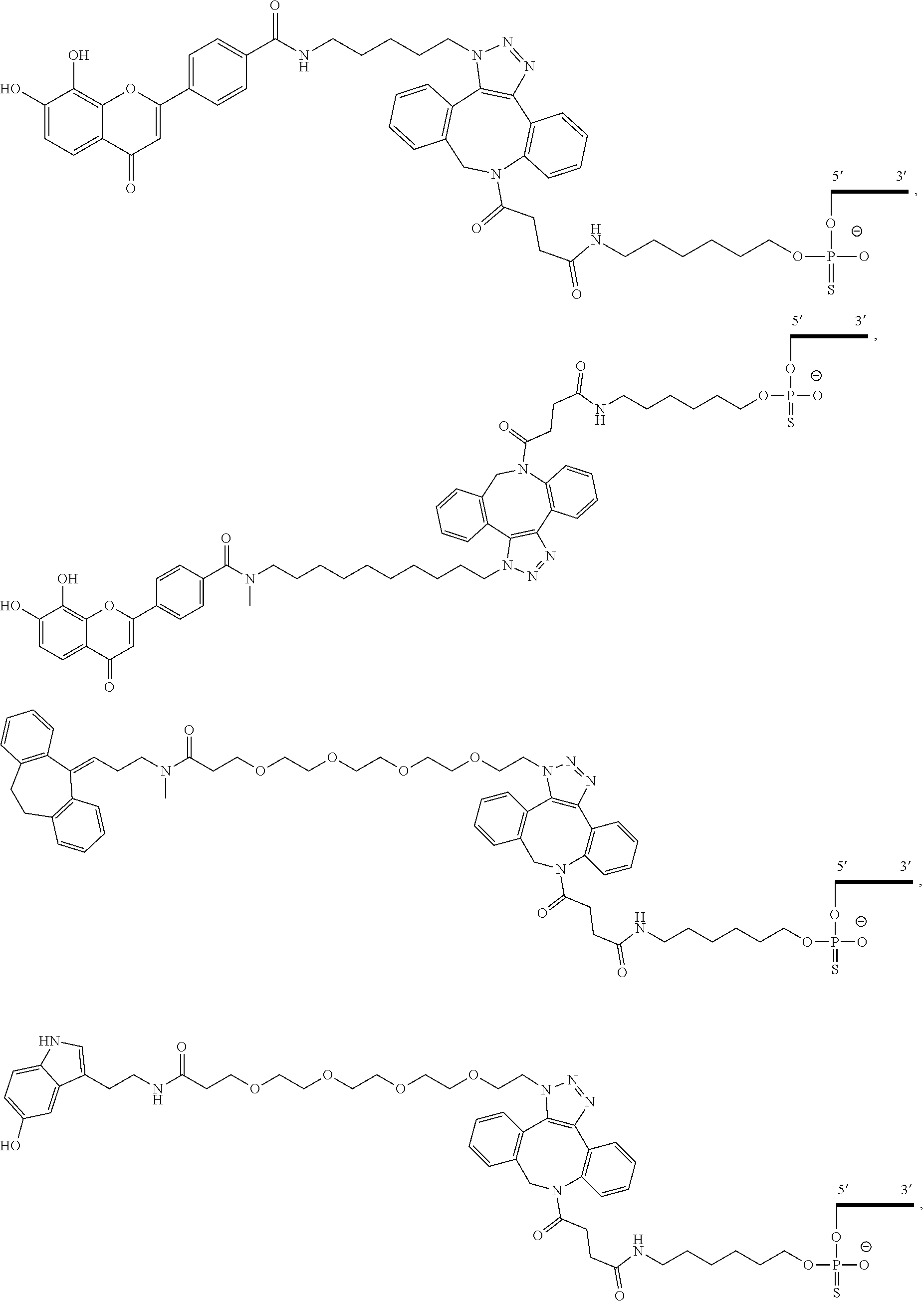

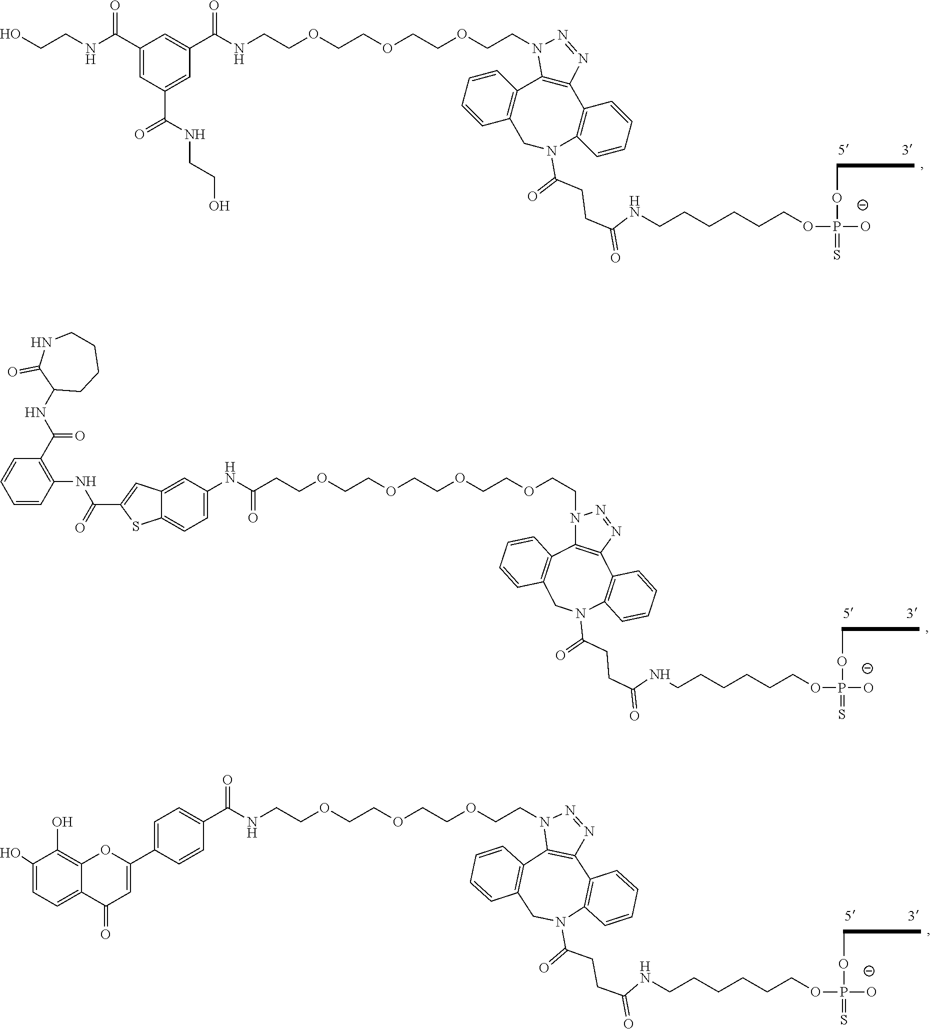

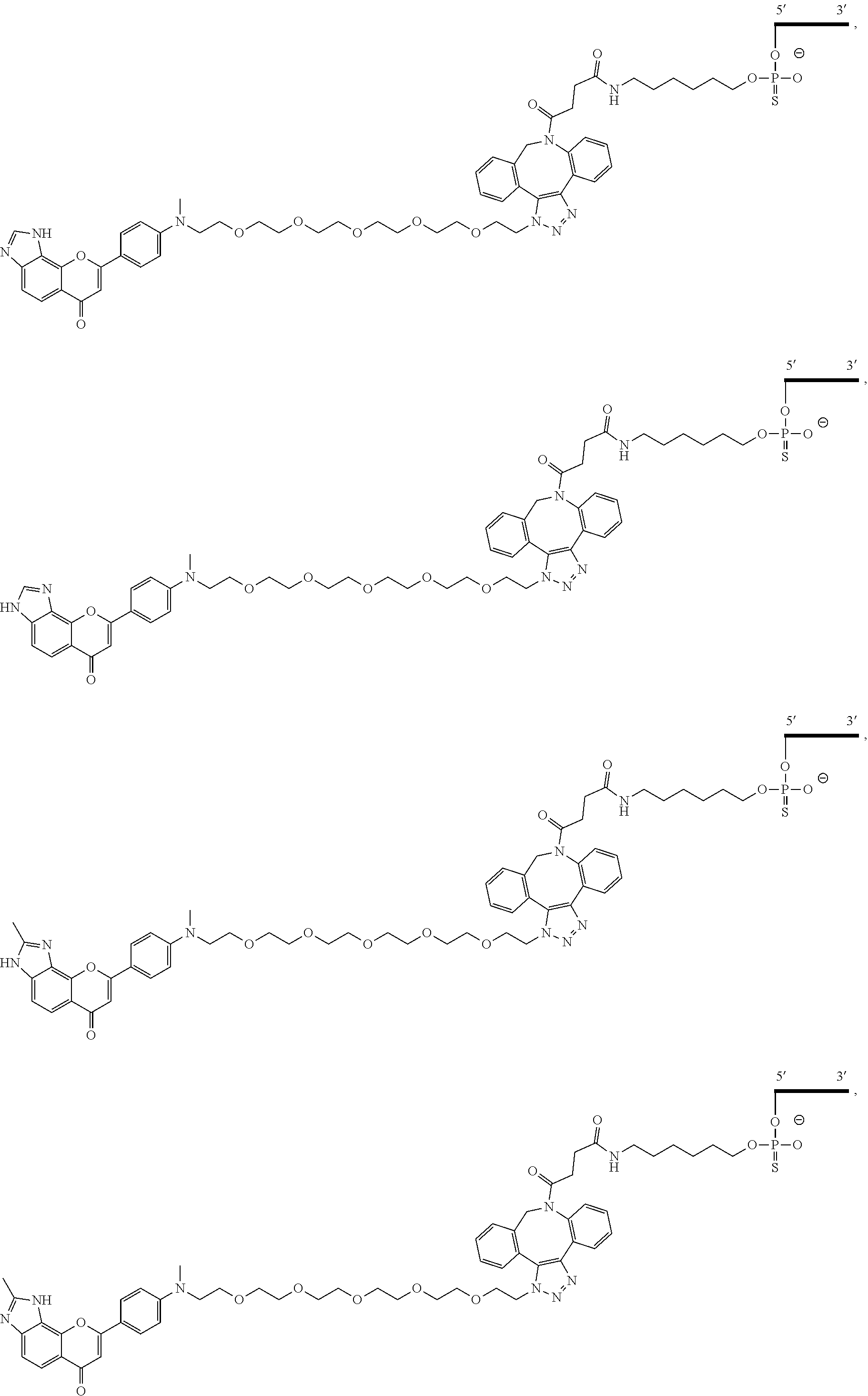

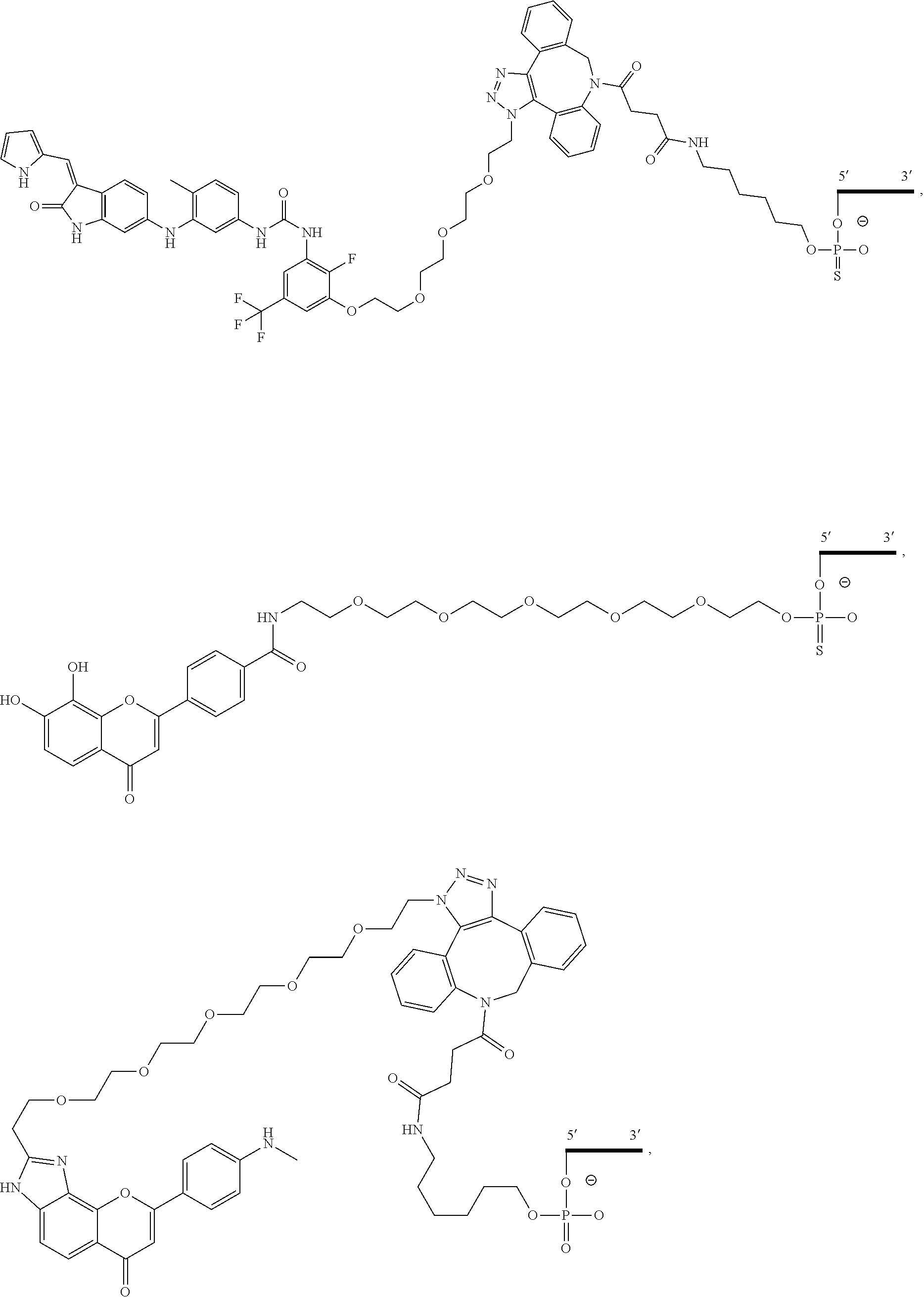

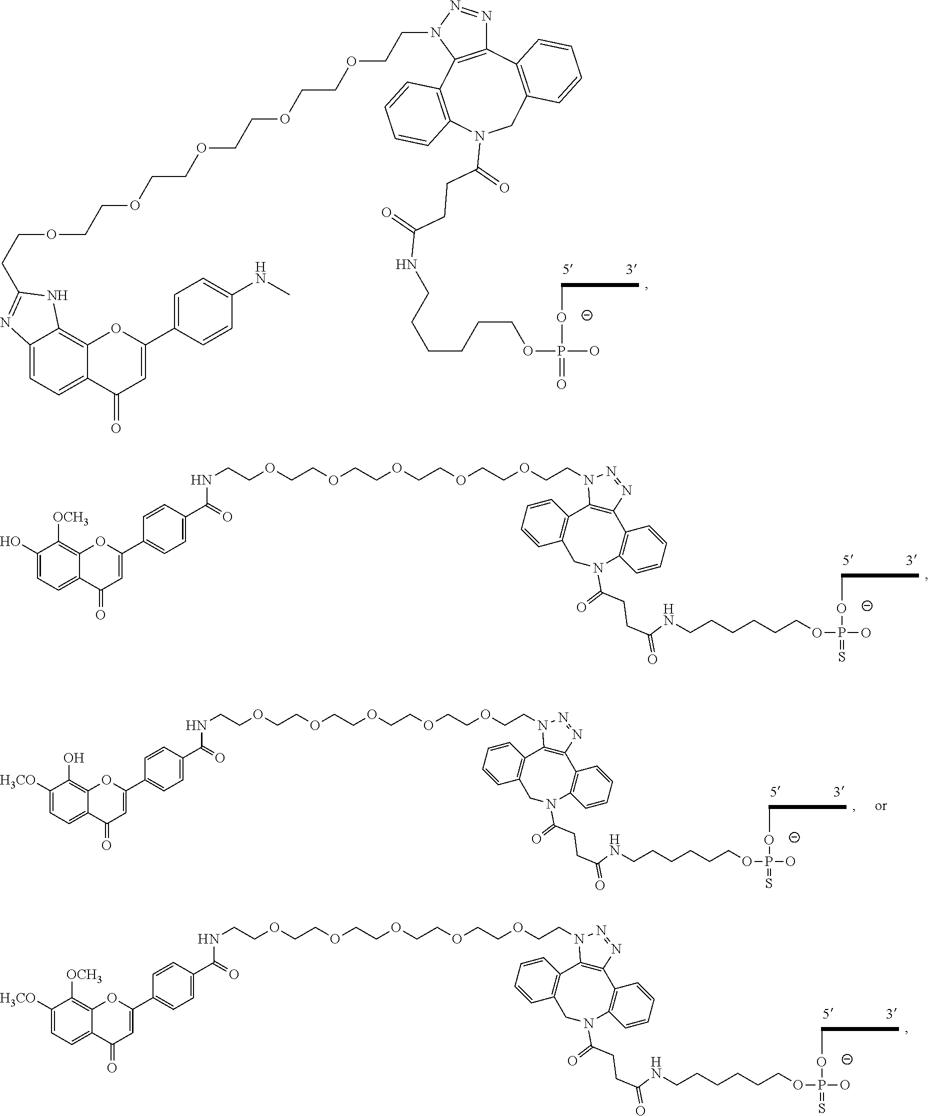

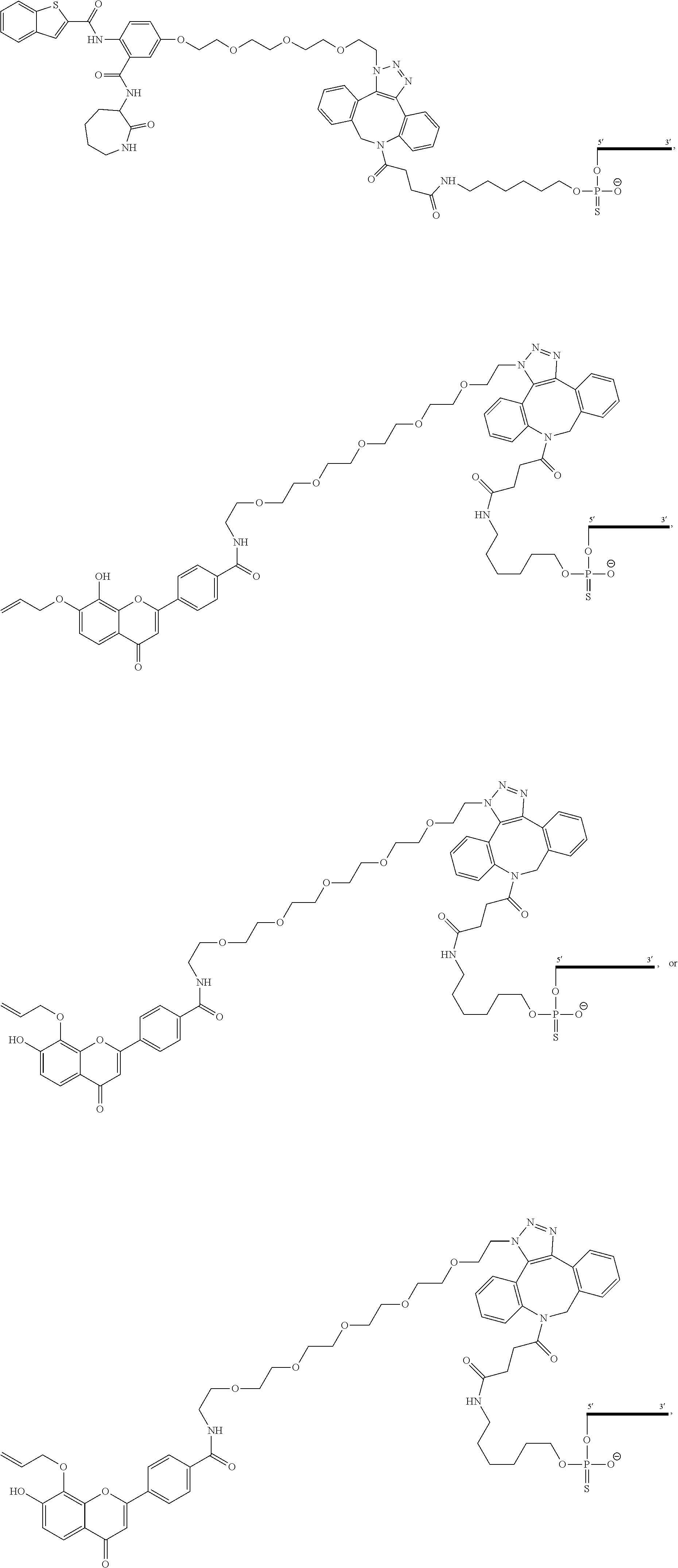

wherein [symbol] is an oligonucleotide.

Embodiment 114. A compound, or a stereoisomer, tautomer, prodrug, or salt thereof, wherein the compound comprises the structure:

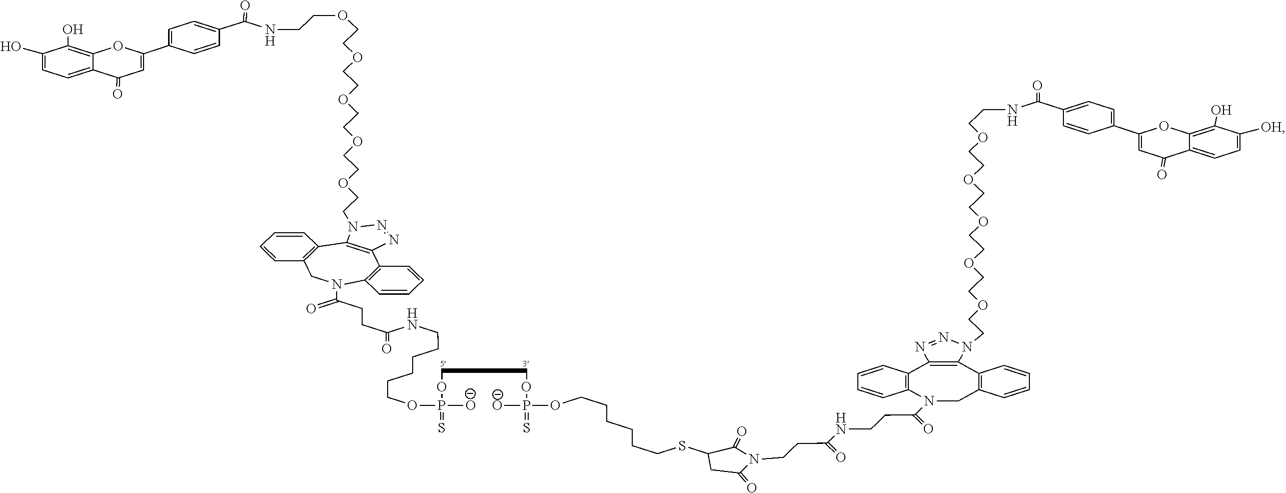

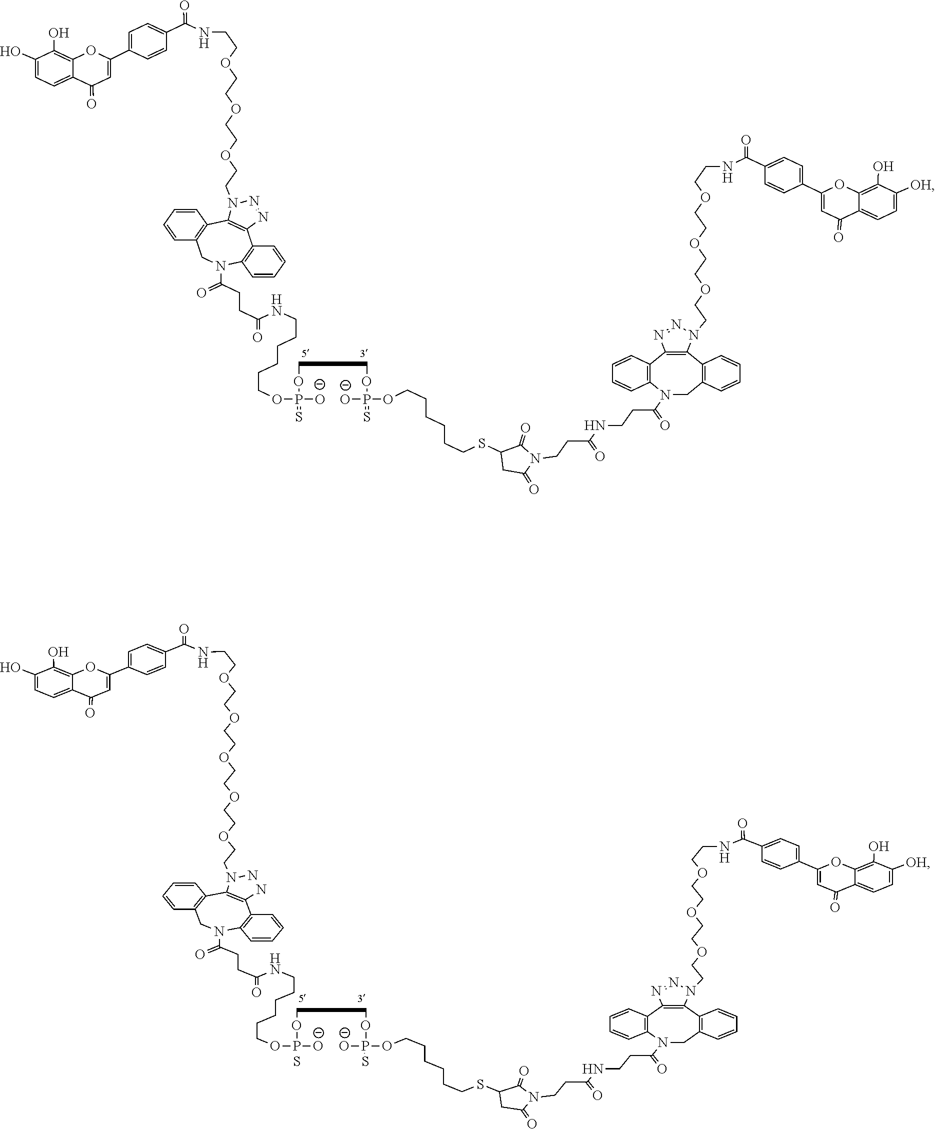

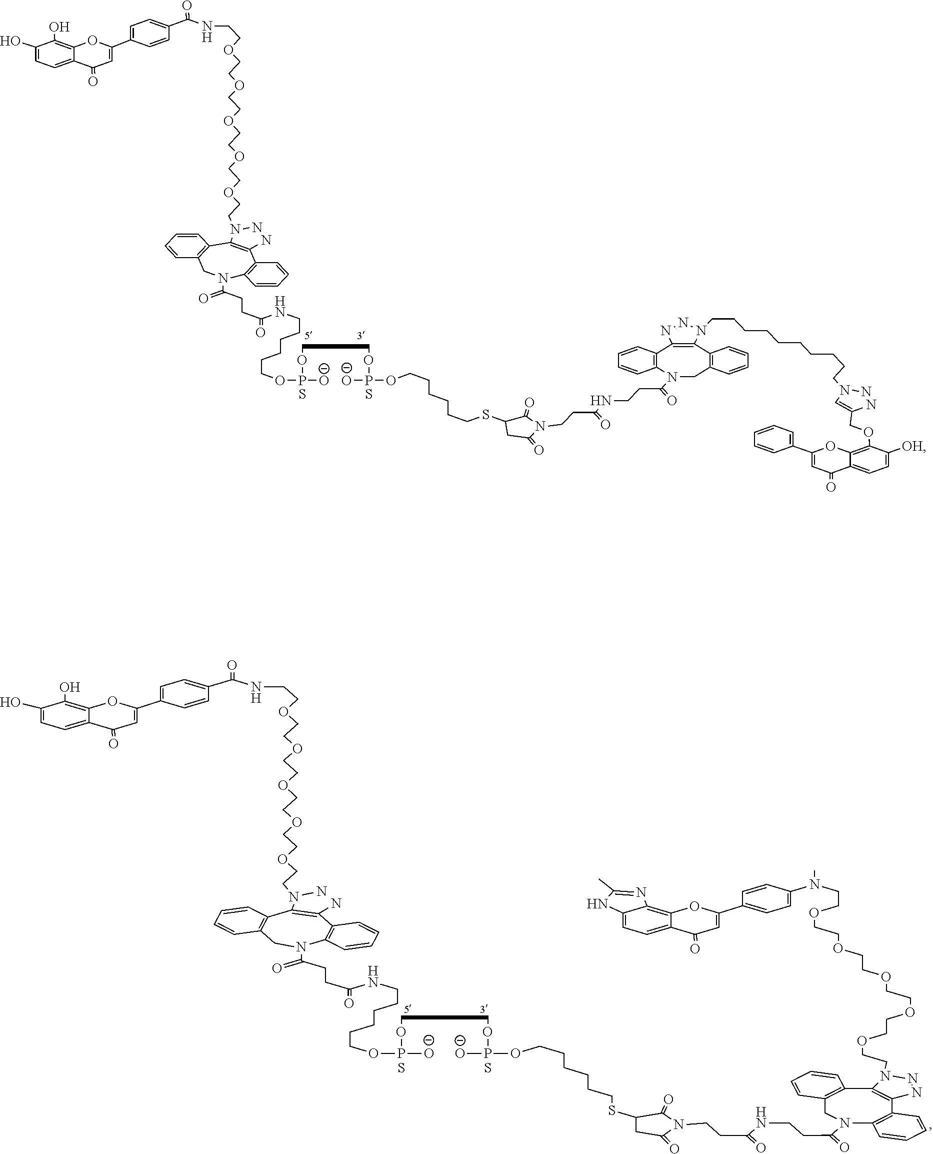

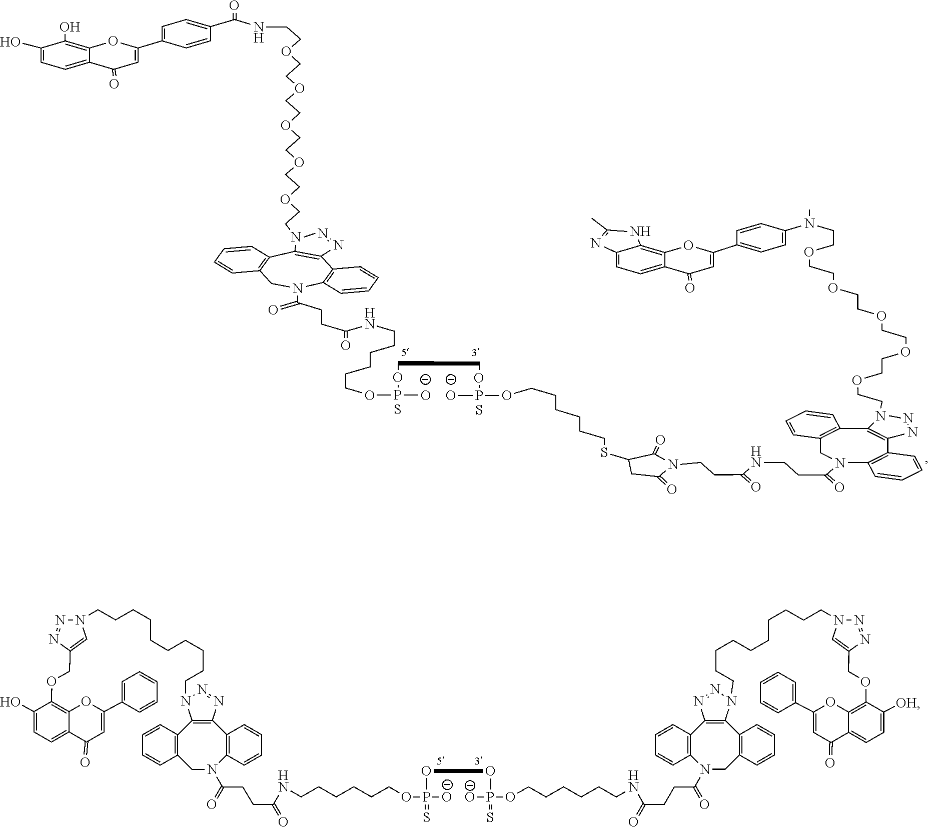

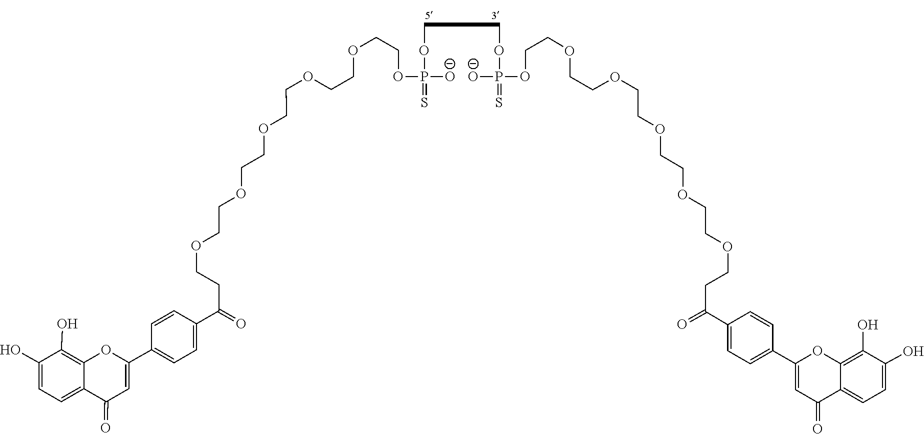

wherein [symbol] is an oligonucleotide.

Embodiment 115. The compound, or a stereoisomer, tautomer, prodrug, or salt thereof, of any one of embodiments 1-114, wherein the compound is a salt.

Embodiment 116. The compound, or a stereoisomer, tautomer, prodrug thereof, of embodiment 115, wherein the salt is a potassium salt or a sodium salt.

Embodiment 117. A composition comprising a compound, or a stereoisomer, tautomer, prodrug, or salt thereof, of any one of embodiments 1-116, and a pharmaceutically acceptable excipient.

Embodiment 118. A method for delivering a therapeutic oligonucleotide to the brain of a subject, comprising administration of a compound, or a stereoisomer, tautomer, prodrug, or salt thereof, of any one of embodiments 1-116, or a composition of embodiment 117, to the subject.

Embodiment 119. The method of embodiment 118, wherein the therapeutic oligonucleotide is delivered to one or more brain regions selected from the group consisting of the striatum, the cerebellum, the brain stem, the hippocampus, the frontal cortex, and the spinal cord.

Embodiment 120. A method for treating or ameliorating a disease, disorder, or symptom thereof in a subject, comprising administration of a compound, or a stereoisomer, tautomer, prodrug, or salt thereof, of any one of embodiments 1-116, or a composition of embodiment 117, to the subject.

Embodiment 121. The method of embodiment 120, wherein the disease, disorder, or symptom thereof is a central nervous system (CNS) disease, disorder, or symptom thereof.

Embodiment 122. The method of embodiment 120 or 121, wherein the disease, disorder, or symptom thereof is Alzheimer's disease, or a symptom thereof.

Embodiment 123. The method of any one of embodiments 118-122, wherein the administration is intrathecal administration or intracerebroventricular (ICV) administration.

Embodiment 124. A method of delivering one or more cargo molecules to a cell or tissue of a subject in vivo, comprising administering to the subject a compound of any one of embodiments 1-116 or a composition of embodiment 117.

Embodiment 125. The method of embodiment 124, wherein the cell or tissue is CNS cell or tissue.

Embodiment 126. A precursor compound, or a stereoisomer, tautomer, or salt thereof, of any one of structural Formulae (A)-(F):

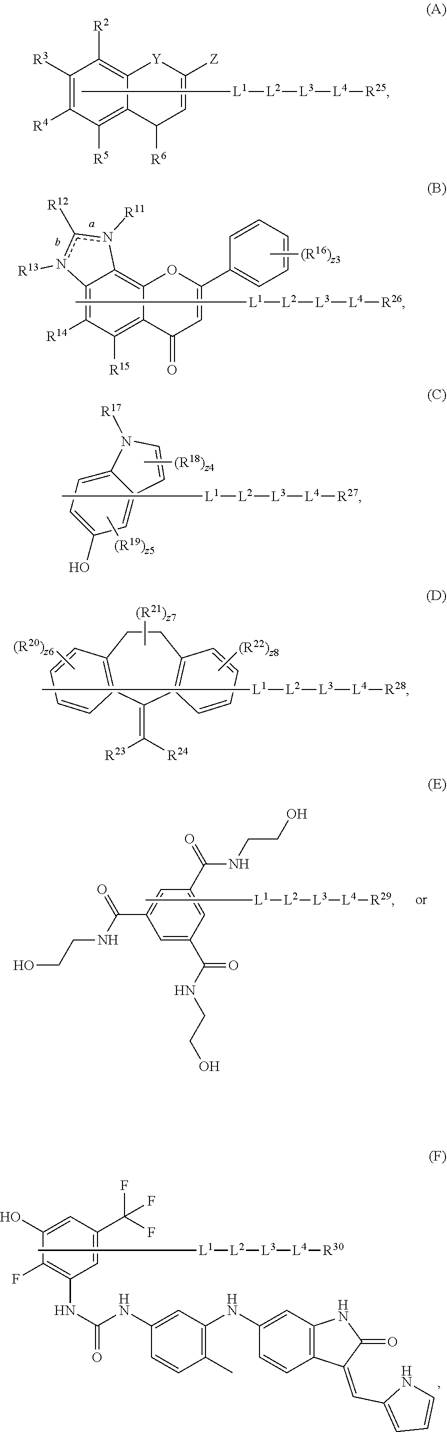

wherein:
each of L¹, L², L³, and L⁴ is independently a linker, bond, or absent;
R² is hydrogen, —OR⁷, —SR⁸, or —NR⁹R¹⁰;
R³ is hydrogen, —OR³¹, —SR³², or —NR³³R³⁴;
R⁴ is hydrogen, —OR³⁵, —SR³⁶, or —NR³⁷R³⁸;
R⁵ is hydrogen, —OR³⁹, —SR⁴⁰, or —NR⁴¹R⁴²;
R⁶ is hydrogen, —OH, optionally substituted —O-alkyl, optionally substituted —OAc, —NH₂, optionally substituted —NHAc, —SH, or ═O;

R⁷, R⁸, R⁹, R¹⁰, R³¹, R³², R³³, R³⁴, R³⁵, R³⁶, R³⁷, R³⁸, R³⁹, R⁴⁰, R⁴¹, and R⁴² are each independently hydrogen, optionally substituted alkyl, optionally substituted heteroalkyl, optionally substituted cycloalkyl, optionally substituted heterocycloalkyl, optionally substituted aryl, optionally substituted heteroaryl;
Y is CH₂, NH, S, or O;
Z is optionally substituted aryl or optionally substituted heteroaryl;
R¹¹ and R₁₃ are each independently absent, hydrogen, or optionally substituted alkyl;
R¹², R¹⁴, and R¹⁵ are each independently hydrogen, optionally substituted alkyl, optionally substituted heteroalkyl, optionally substituted cycloalkyl, optionally substituted heterocycloalkyl, optionally substituted aryl, or optionally substituted heteroaryl;
R¹⁶ is hydrogen, halogen, —CN, —N₃, —SOₙ₁₆R^{1A}, —SOᵥ₁₆NR^{16B}R^{16C}, —NHNR^{16B}R^{16C}, —ONR^{16B}R^{16C}, —NHC(O)NHNR^{16B}R^{16C}, —NHC(O)NR^{16B}R^{16C}, —N(O)ₘ₁₆, —NR^{16B}R^{16C}, —C(O)R^{16D}, —C(O)OR^{16D}, —C(O)NR^{16B}R^{16C}, —OR^{16A}, —NR^{16B}SO₂R^{16A}, —NR^{16B}C(O)R^{16D}; —NR^{16B}C(O)OR^{16D}, —NR^{16B}OR^{16D}, optionally substituted alkyl, optionally substituted heteroalkyl, optionally substituted cycloalkyl, optionally substituted heterocycloalkyl, optionally substituted aryl, or optionally substituted heteroaryl;
[symbol] and [symbol] are each independently a single bond or a double bond, wherein if [symbol] is a single bond, then [symbol] is a double bond and R¹³ is absent; and further wherein if [symbol] is a single bond, then [symbol] is a double bond and R¹¹ is absent;
R^{16A}, R^{16B}, R^{16C}, R^{16D} are each independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{16B} and R^{16C} substituents bonded to the same nitrogen atom may optionally be joined to form a substituted or unsubstituted heterocycloalkyl or substituted or unsubstituted heteroaryl;
R¹⁷, R¹⁸, and R¹⁹ are each independently hydrogen, optionally substituted alkyl, optionally substituted heteroalkyl, optionally substituted cycloalkyl, optionally substituted heterocycloalkyl, optionally substituted aryl, or optionally substituted heteroaryl;
R²⁰ is hydrogen, halogen, —CN, —N₃, —SOₙ₂₀R^{1A}, —SOᵥ₂₀NR^{20B}R^{20C}, —NHNR^{20B}R^{20C}, —ONR^{20B}R^{20C}, —NHC(O)NHNR^{20B}R^{20C}, —NHC(O)NR^{20B}R^{20C}, —N(O)ₘ₂₀, —NR^{20B}R^{20C}, —C(O)R^{20D}, —C(O)OR^{20D}, —C(O)NR^{20B}R^{20C}, —OR^{20A}, —NR^{20B}SO₂R^{20A}, —NR^{20B}C(O)R^{20D};
—NR^{20B}C(O)OR^{20D}, —NR^{20B}OR^{20D}, optionally substituted alkyl, optionally substituted heteroalkyl, optionally substituted cycloalkyl, optionally substituted heterocycloalkyl, optionally substituted aryl, or optionally substituted heteroaryl;
R²¹ is hydrogen, halogen, —CN, —N₃, —SOₙ₂₁R^{1A}, —SOᵥ₂₁NR^{21B}R^{21C}, —NHNR^{21B}R^{21C}, —ONR^{21B}R^{21C}, —NHC(O)NHNR^{21B}R^{21C}, —NHC(O)NR^{21B}R^{21C}, —N(O)ₘ₂₁, —NR^{21B}R^{21C}, —C(O)R^{21D}, —C(O)OR^{21D}, —C(O)NR^{21B}R^{21C}, —OR^{21A}, —NR^{21B}SO₂R^{21A}, —NR^{21B}C(O)R^{21D};
—NR^{21B}C(O)OR^{21D}, —NR^{21B}OR^{21D}, optionally substituted alkyl, optionally substituted heteroalkyl, optionally substituted cycloalkyl, optionally substituted heterocycloalkyl, optionally substituted aryl, or optionally substituted heteroaryl;
R²² and R²³ are each independently hydrogen, optionally substituted alkyl, optionally substituted heteroalkyl, optionally substituted cycloalkyl, optionally substituted heterocycloalkyl, optionally substituted aryl, or optionally substituted heteroaryl;
R²⁴ is hydrogen, halogen, —CN, —N₃, —SOₙ₂₄R^{1A}, —SOᵥ₂₄NR^{24B}R^{24C}, —NHNR^{24B}R^{24C}, —ONR^{24B}R^{24C}, —NHC(O)NHNR^{24B}R^{24C}, —NHC(O)NR^{24B}R^{24C}, —N(O)ₘ₂₄, —NR^{24B}R^{24C}, —C(O)R^{24D}, —C(O)OR^{24D}, —C(O)NR^{24B}R^{24C}, —OR^{24A}, —NR^{24B}SO₂R^{24A}, —NR^{24B}C(O)R^{24D};
—NR^{24B}C(O)OR^{24D}, —NR^{24B}OR^{24D}, optionally substituted alkyl, optionally substituted heteroalkyl, optionally substituted cycloalkyl, optionally substituted heterocycloalkyl, optionally substituted aryl, or optionally substituted heteroaryl;
R^{20A}, R^{20B}, R^{20C}, R^{20D}, R^{21A}, R^{21B}, R^{21C}, R^{21D}, R^{24A}, R^{24B}, R^{24C}, and R^{24D} are each independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{20B}, R^{20C}, R^{21B}, R^{21C}, R^{24B}, R^{24C}, R^{24B}, and R^{24C} substituents bonded to the same nitrogen atom may optionally be joined to form a substituted or unsubstituted heterocycloalkyl or substituted or unsubstituted heteroaryl;
n16, n20, n21, n23, n24, z6, and z8 are each independently 0, 1, 2, 3, or 4;
v16, v20, v21, m16, m20, m21, and m24 are each independently 1 or 2; and
z3 is 0, 1, 2, 3, 4, or 5;
z4 and z7 are each independently 0, 1, or 2;
z5 is 0, 1, 2, or 3;
z6 and z8 are each independently 0, 1, 2, 3, or 4 and
R²⁵, R²⁶, R²⁷, R²⁸, R²⁹, R³⁰ are each independently —N₃.

Embodiment 127. A method for making a compound, or a stereoisomer, tautomer, prodrug, or salt thereof, of any one of embodiments 1-116, comprising contacting the precursor compound of embodiment 126 with a compound of structural Formula (G) and/or (H):

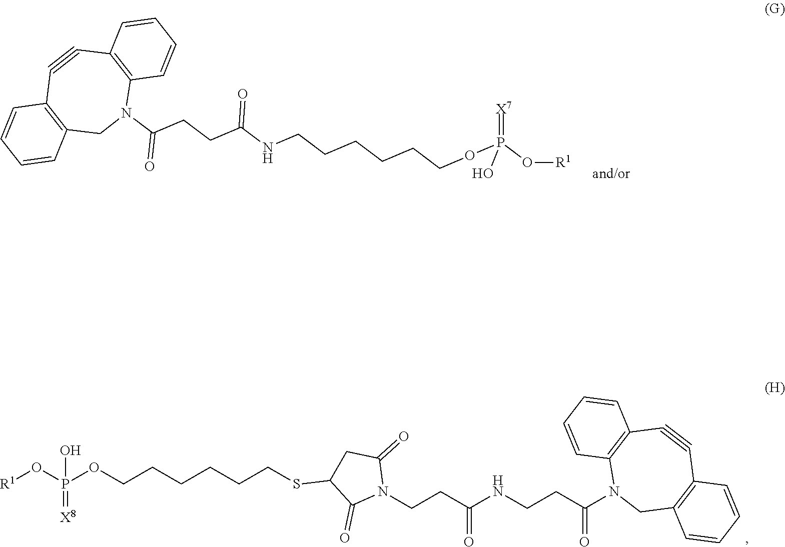

or a salt thereof,
wherein:
X¹ and X⁸ are each independently O or S; and
R¹ is an oligonucleotide, a protecting group, a small molecule, a protein, an antibody, or a peptide.

Embodiment 128. A compound, or a stereoisomer, tautomer, prodrug, or salt thereof, wherein the compound comprises the structure:

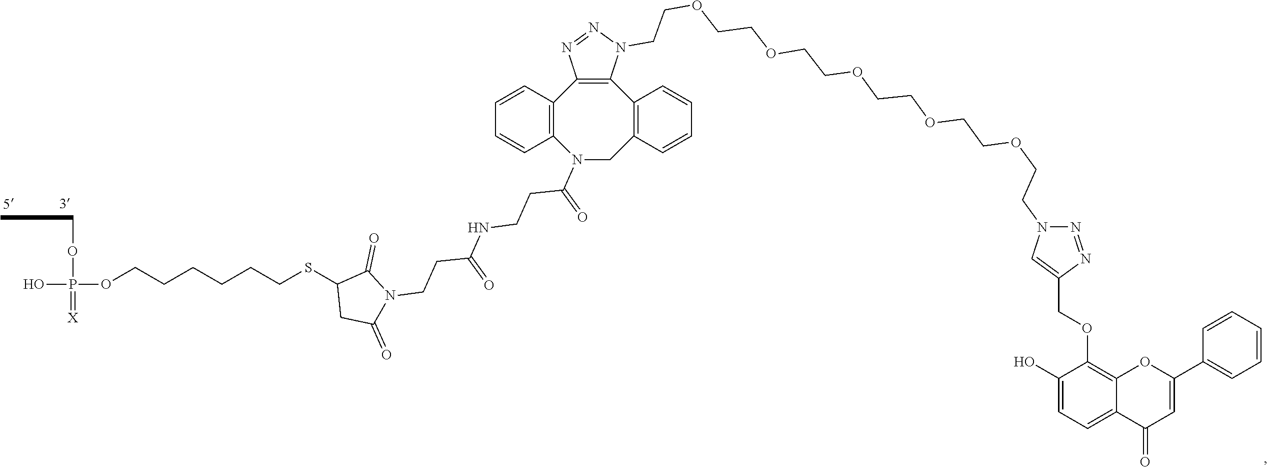

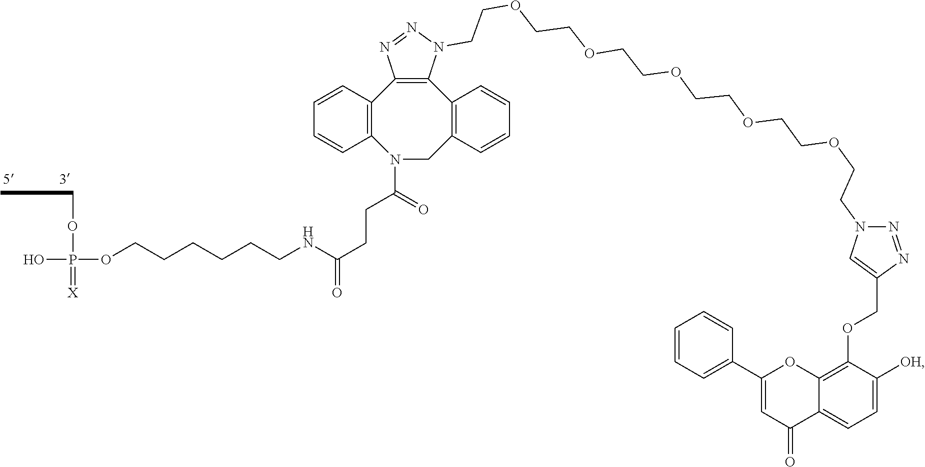

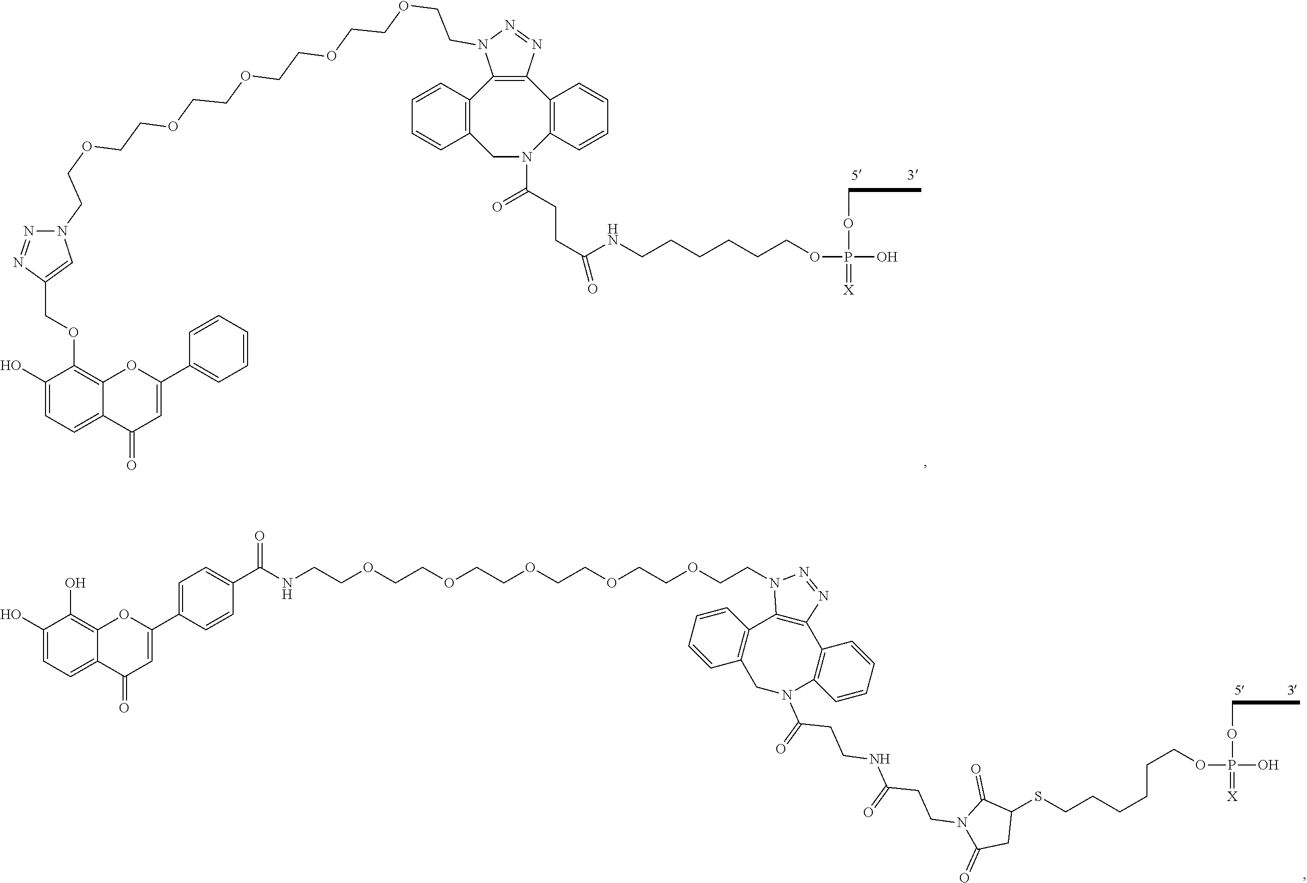

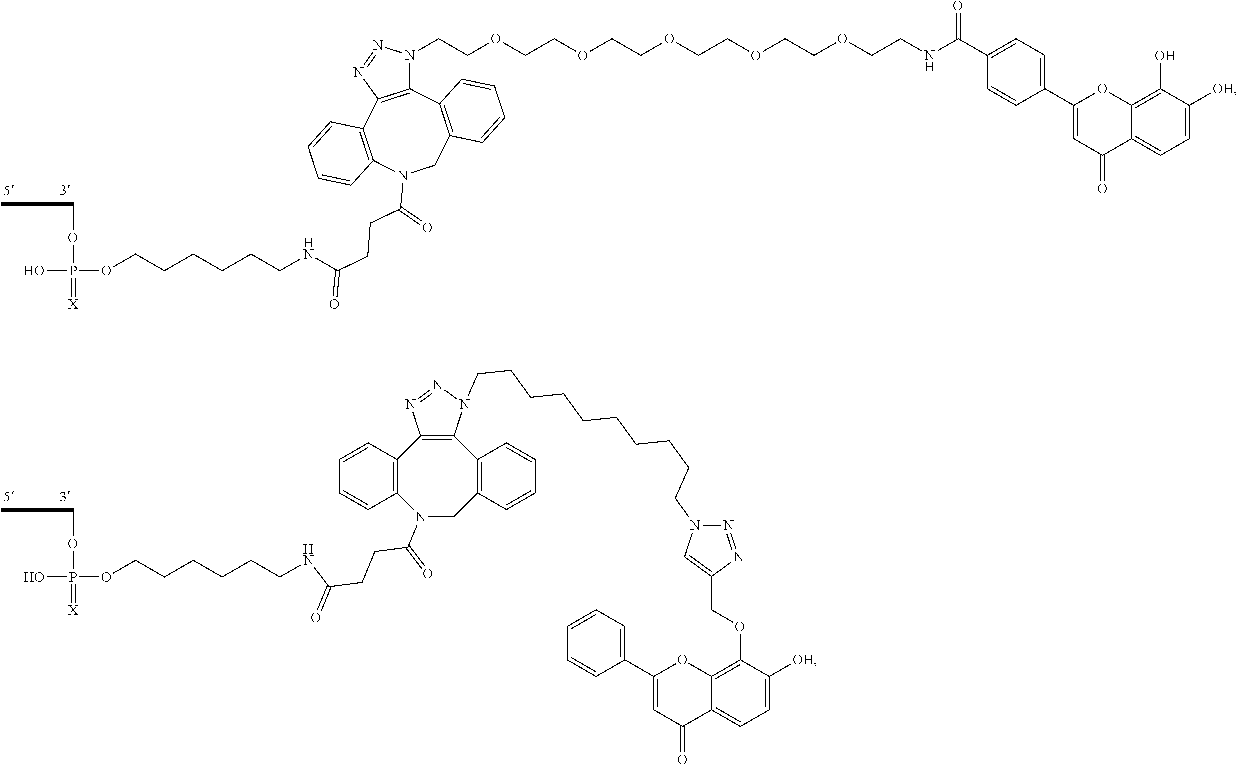

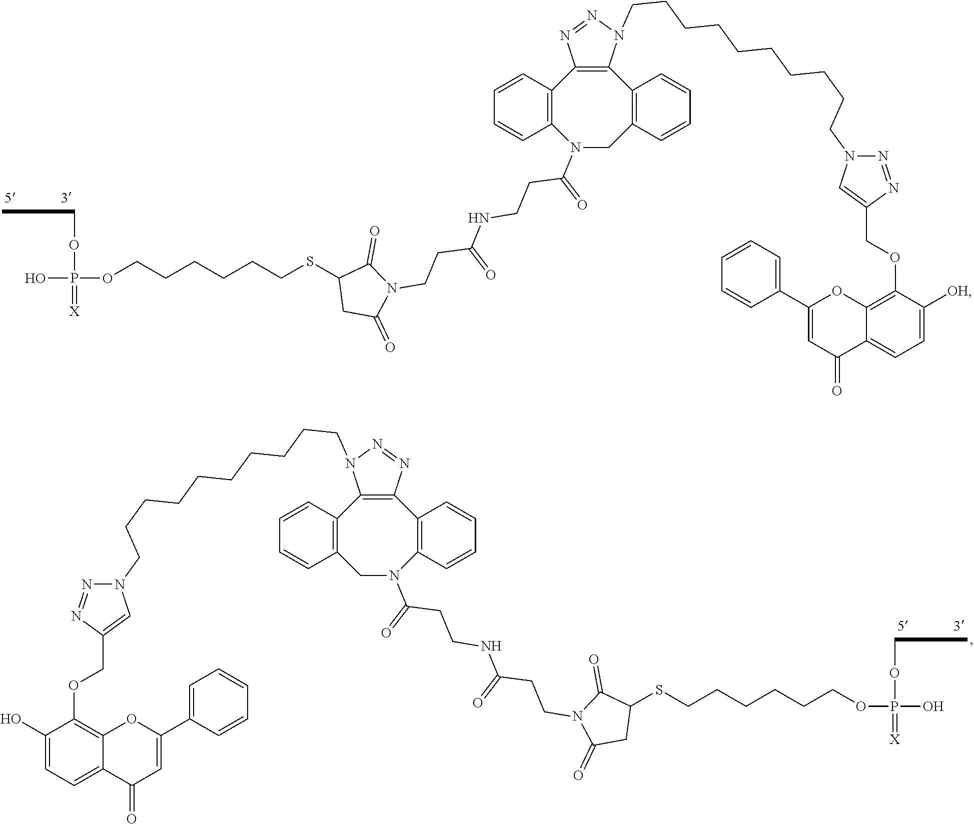

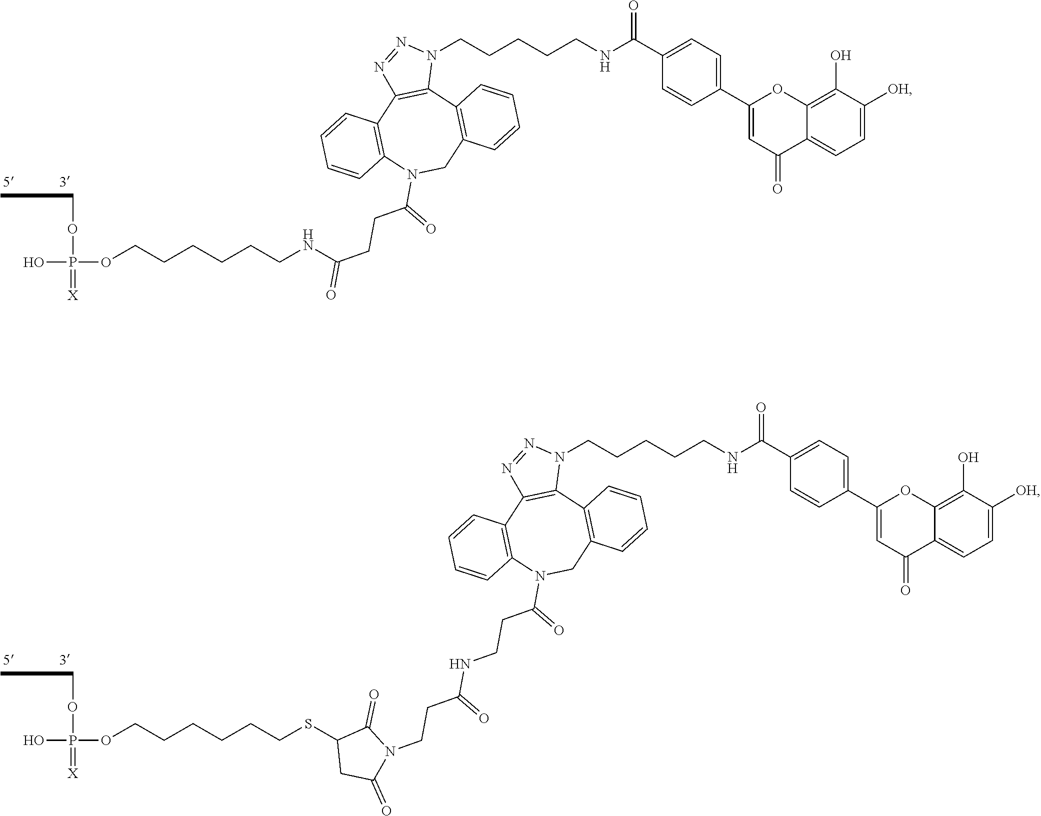

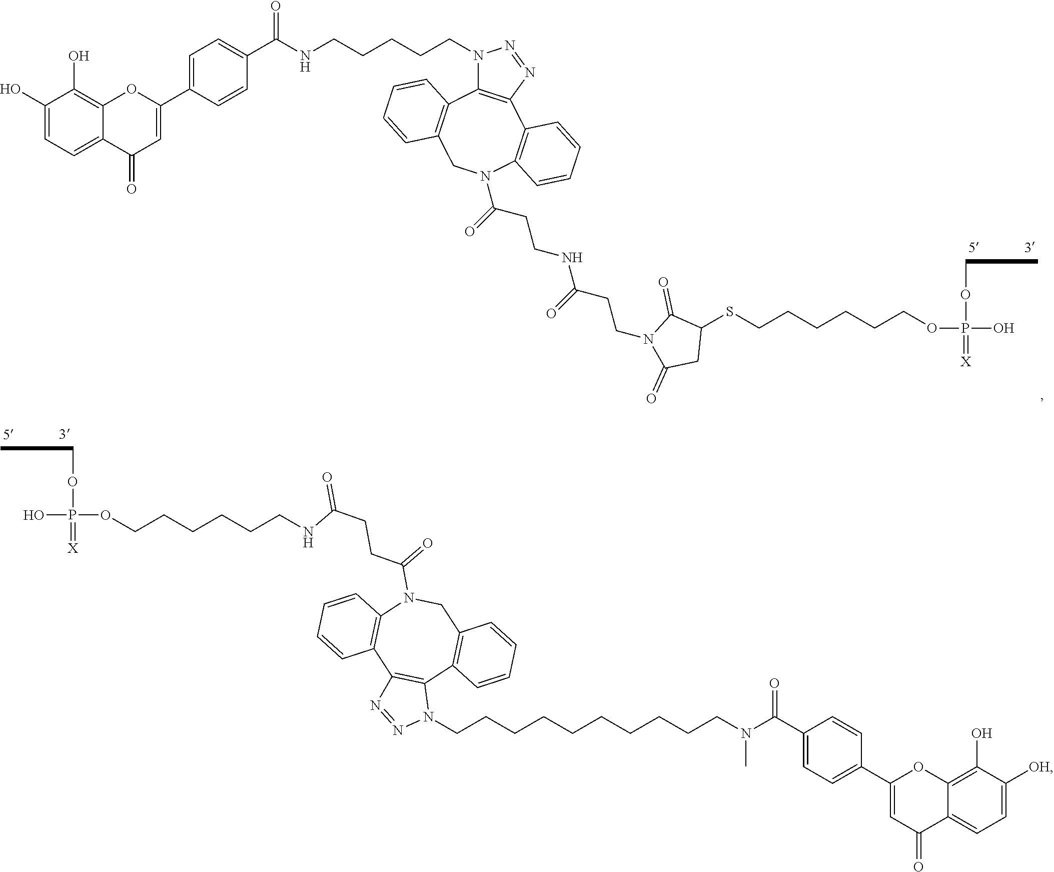

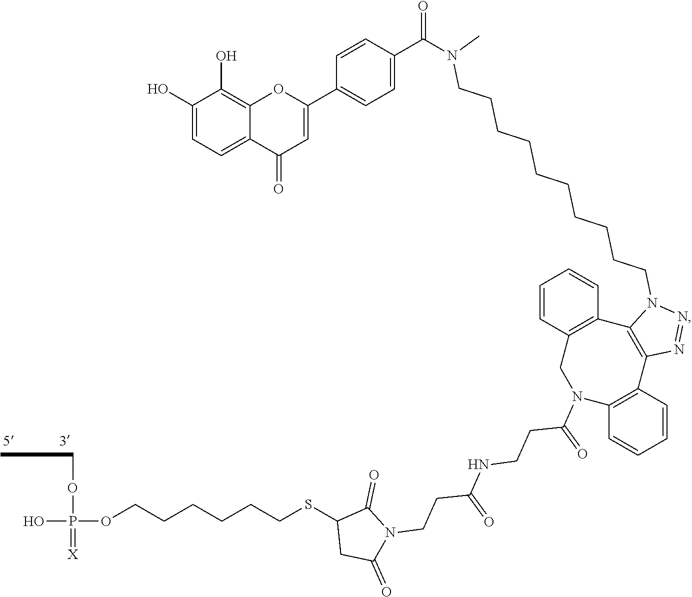

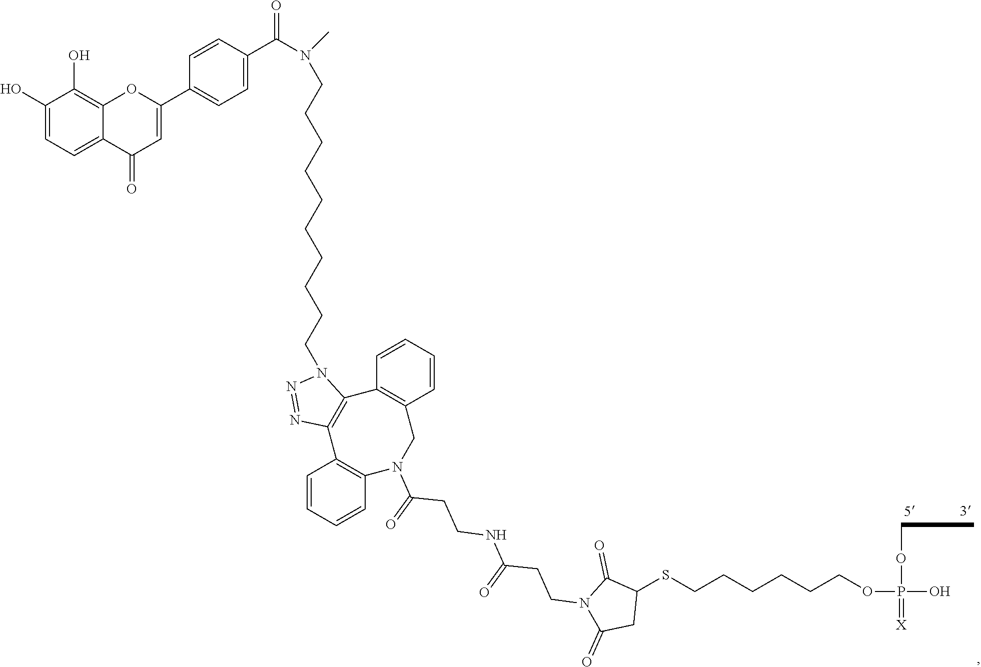

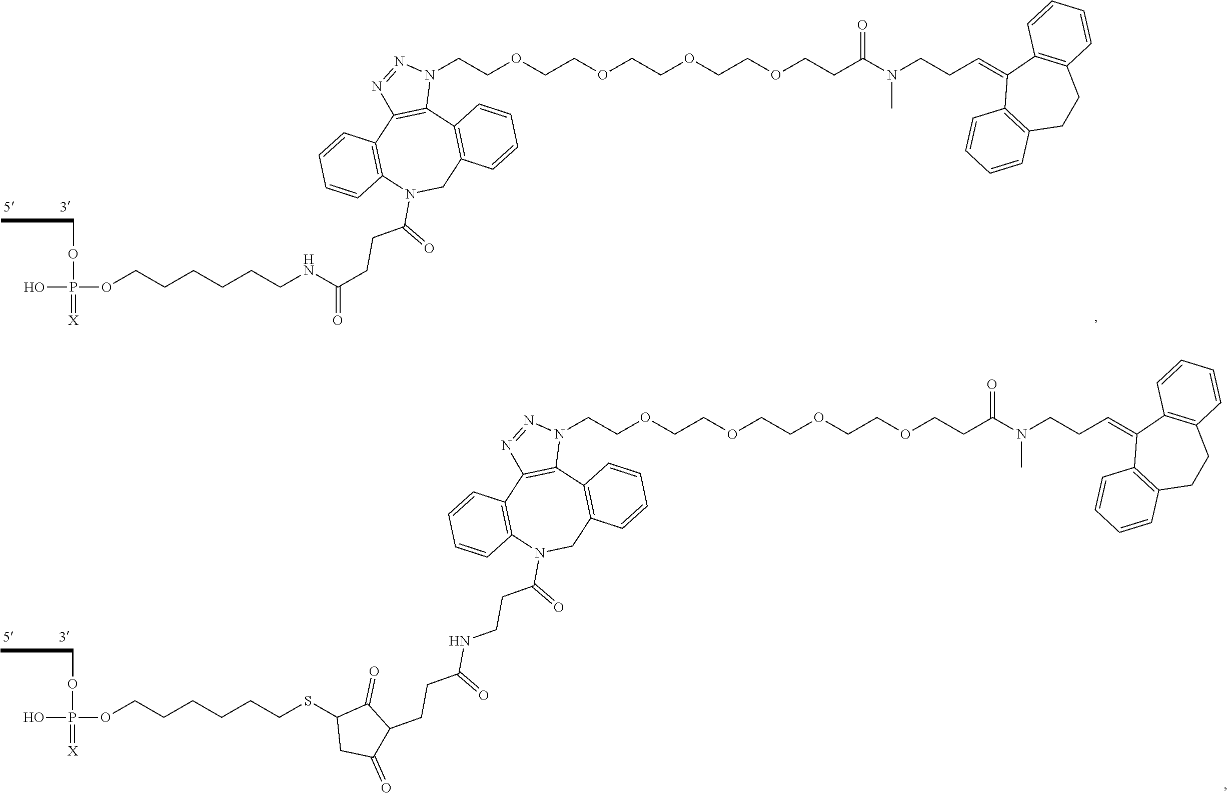

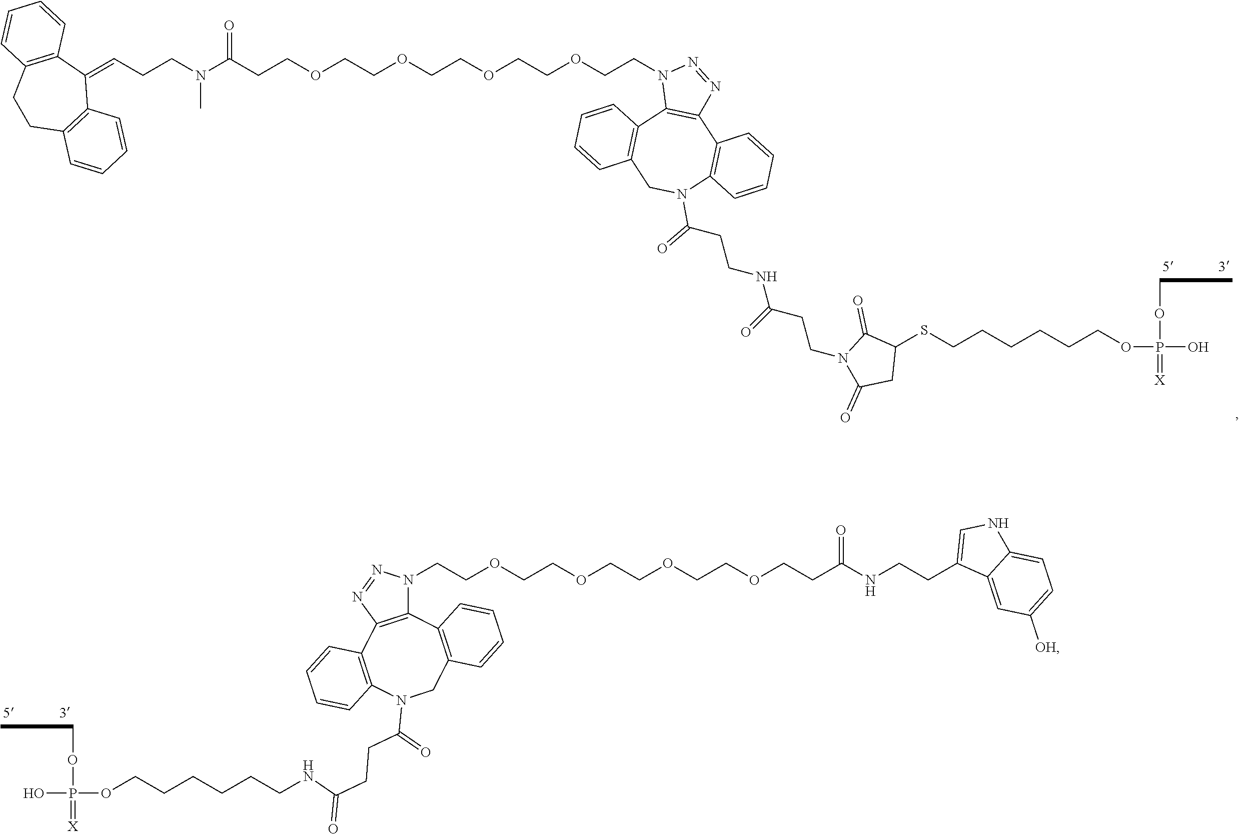

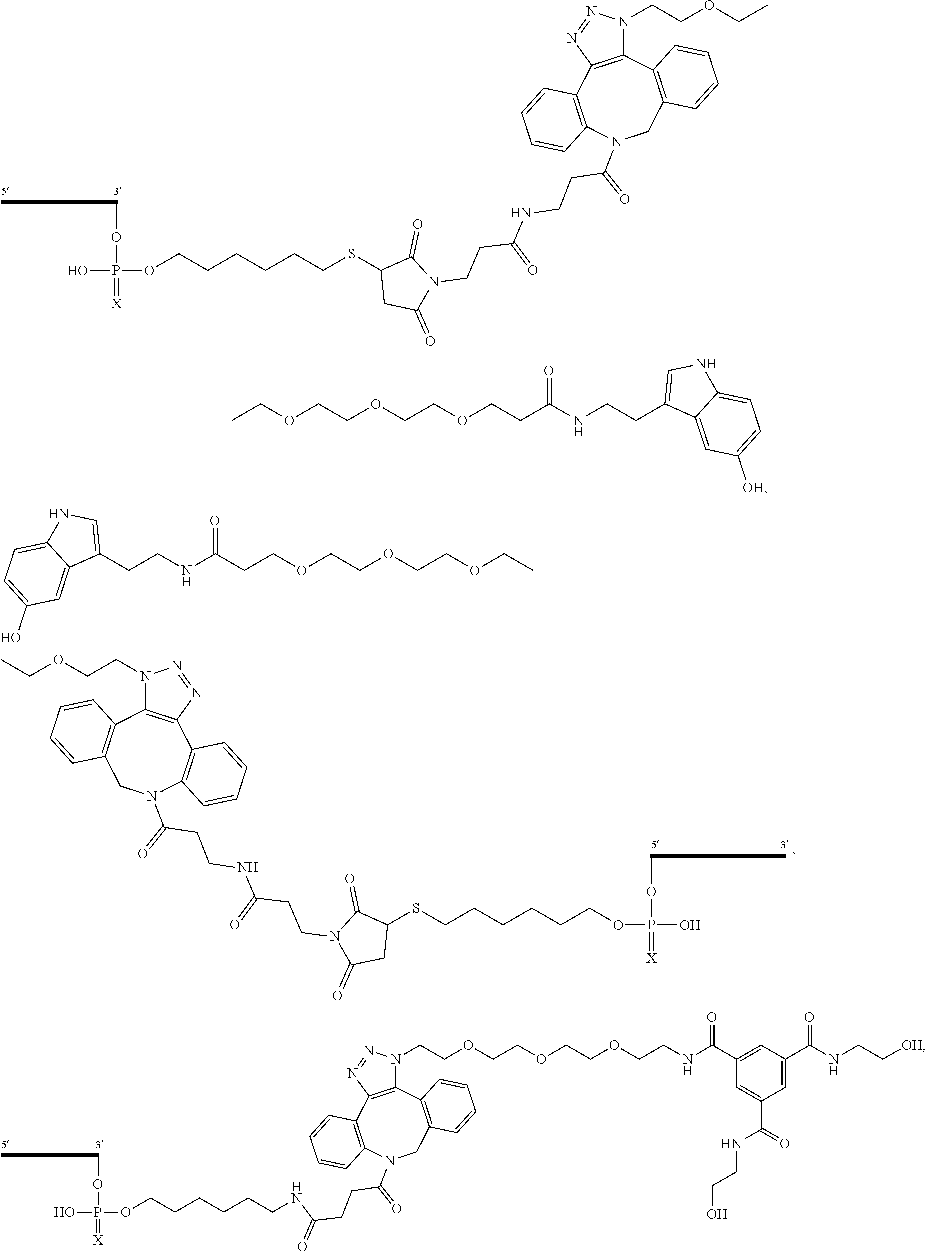

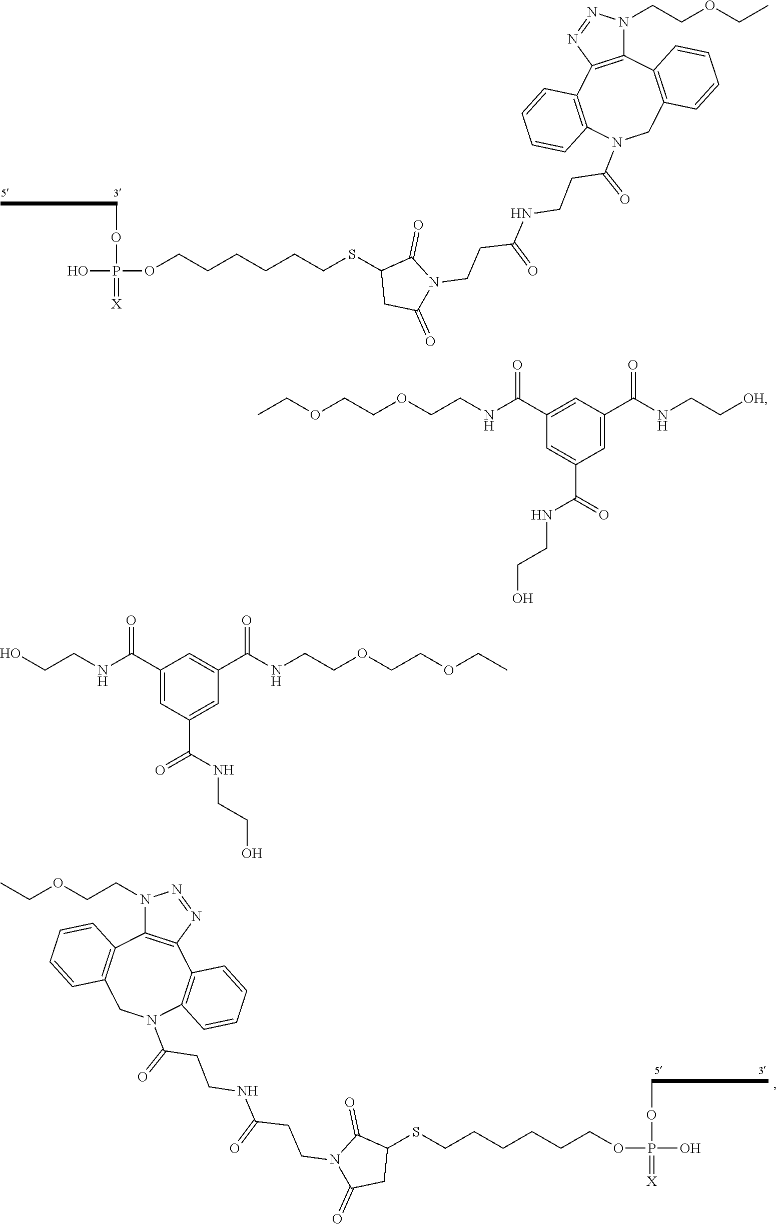

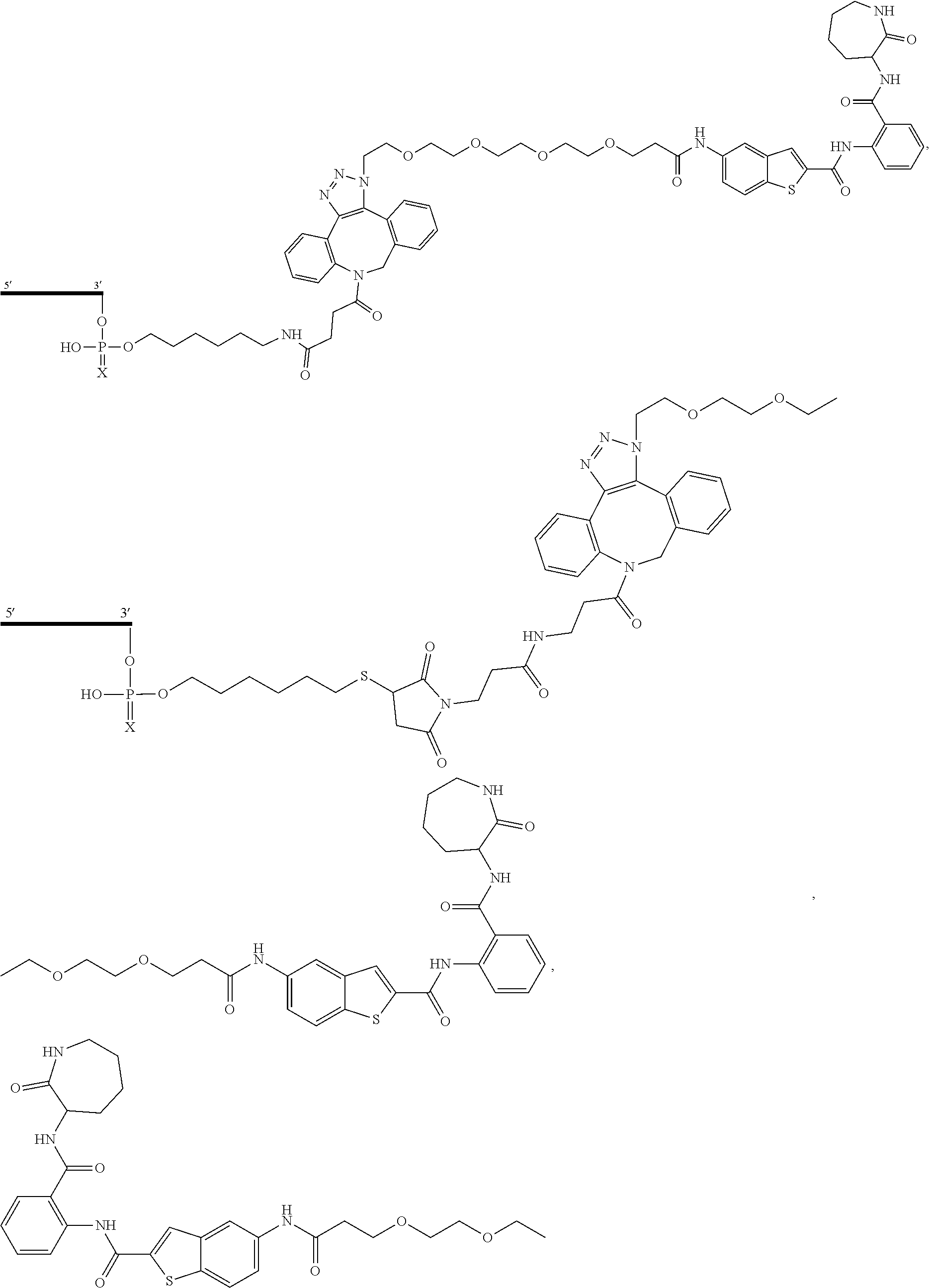

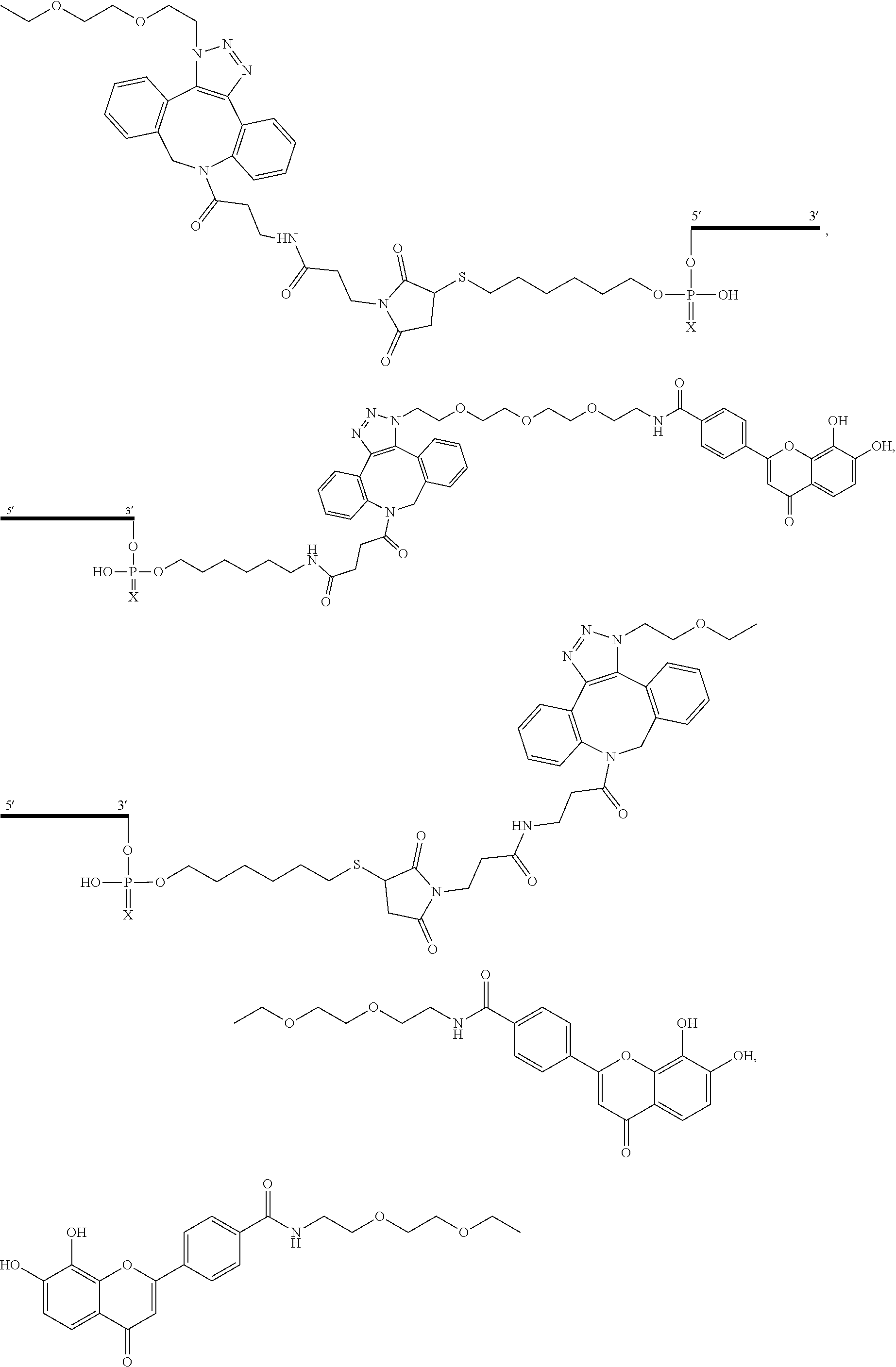

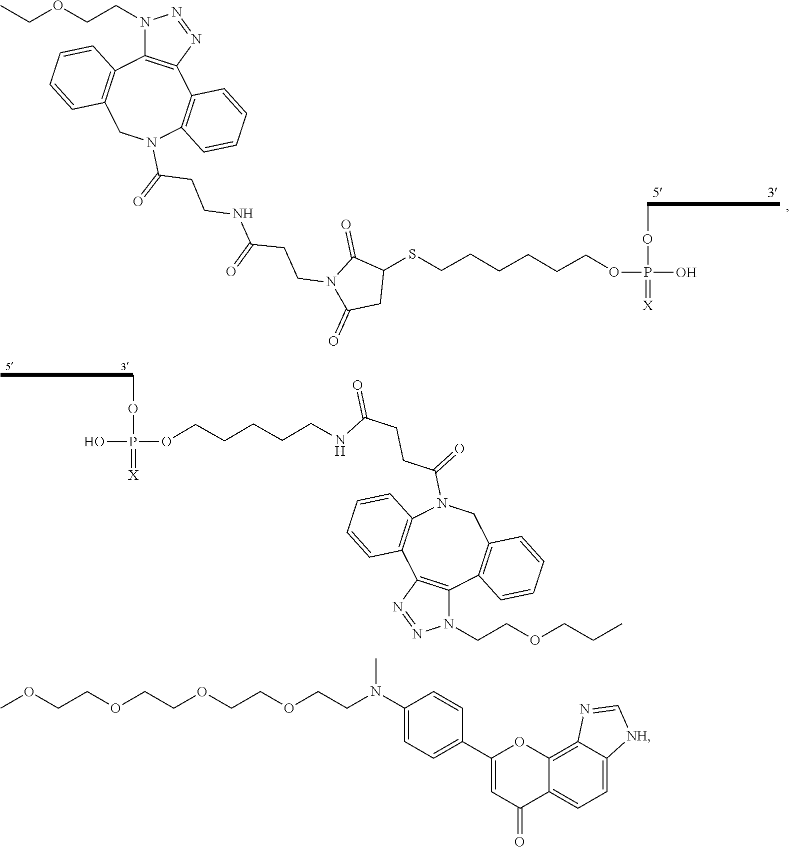

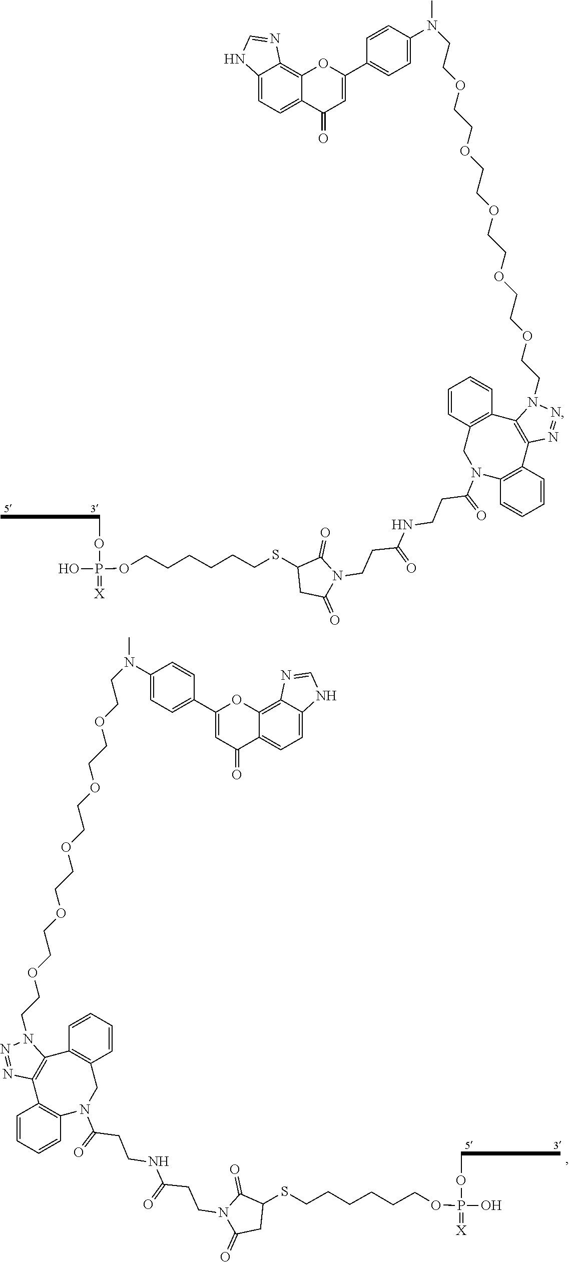

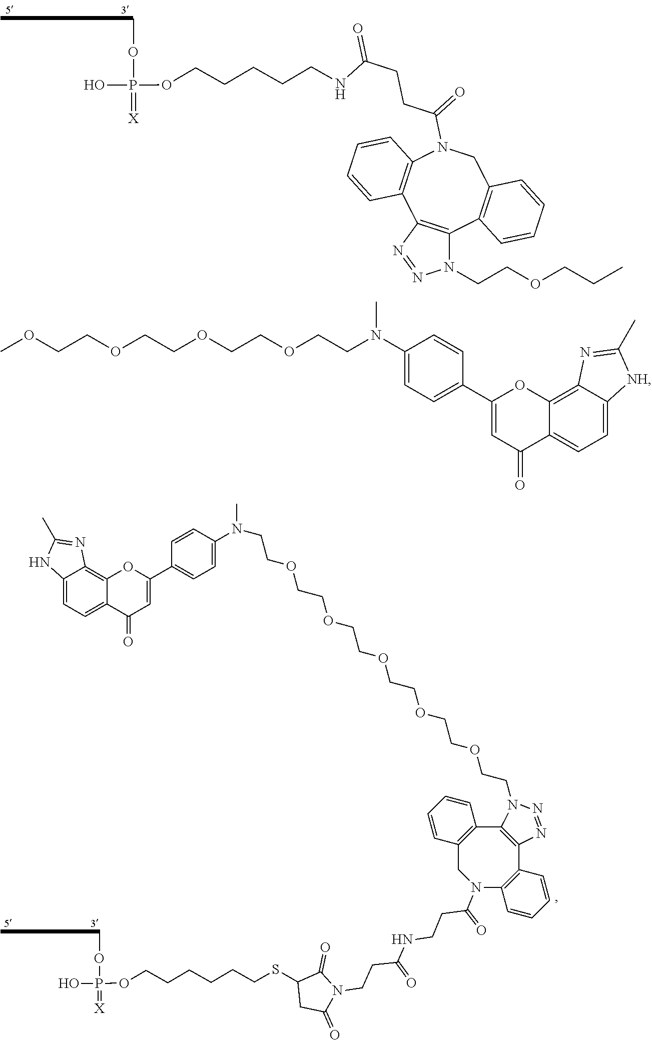

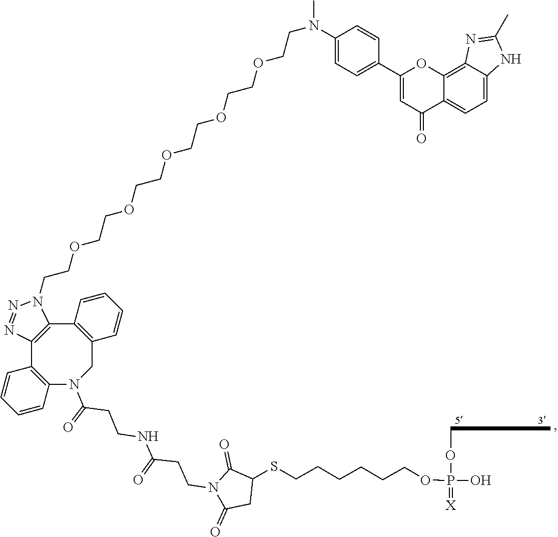

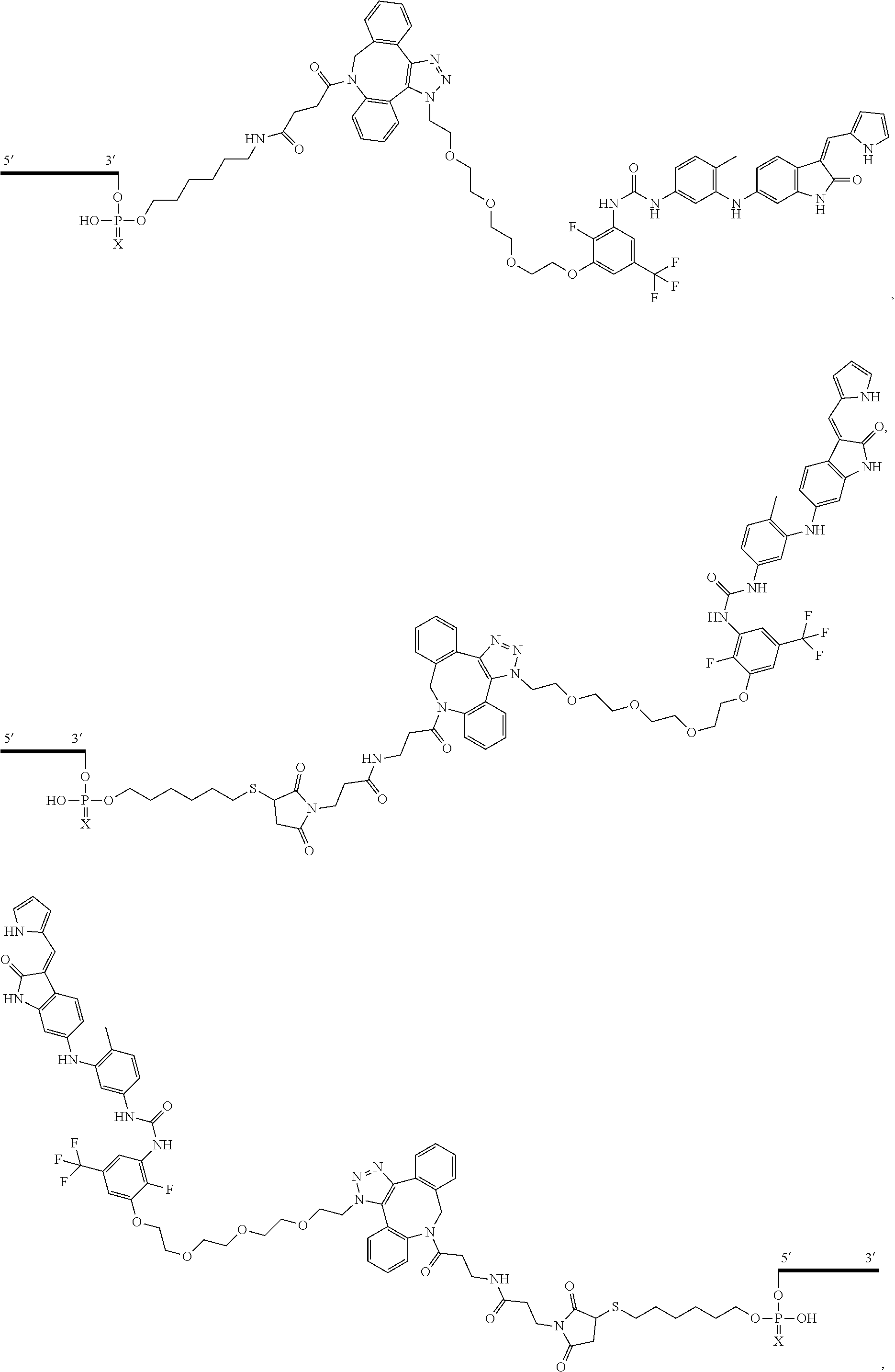

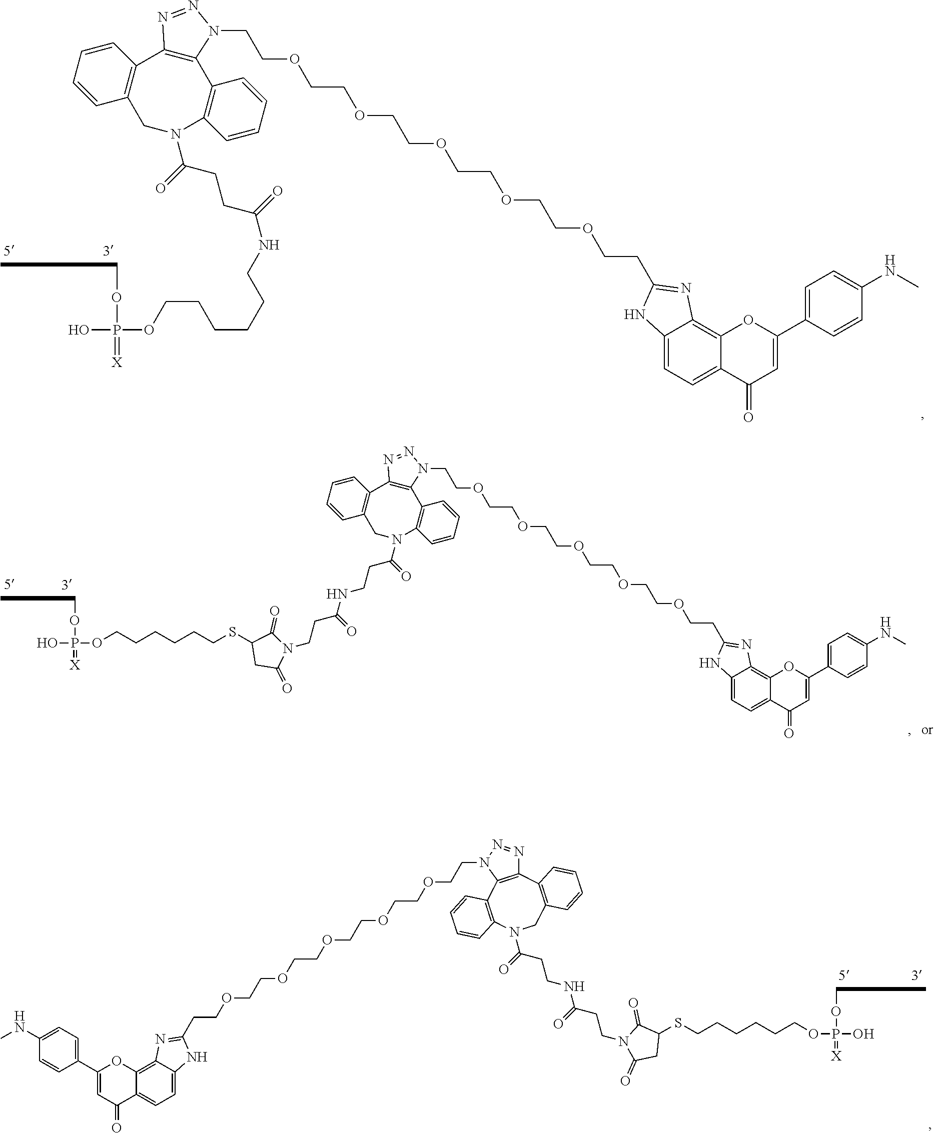

wherein:
X is S or O; and
[symbol] is an oligonucleotide.

Embodiment 129. A precursor compound, or a stereoisomer, tautomer, or salt thereof, wherein the compound comprises the structure:

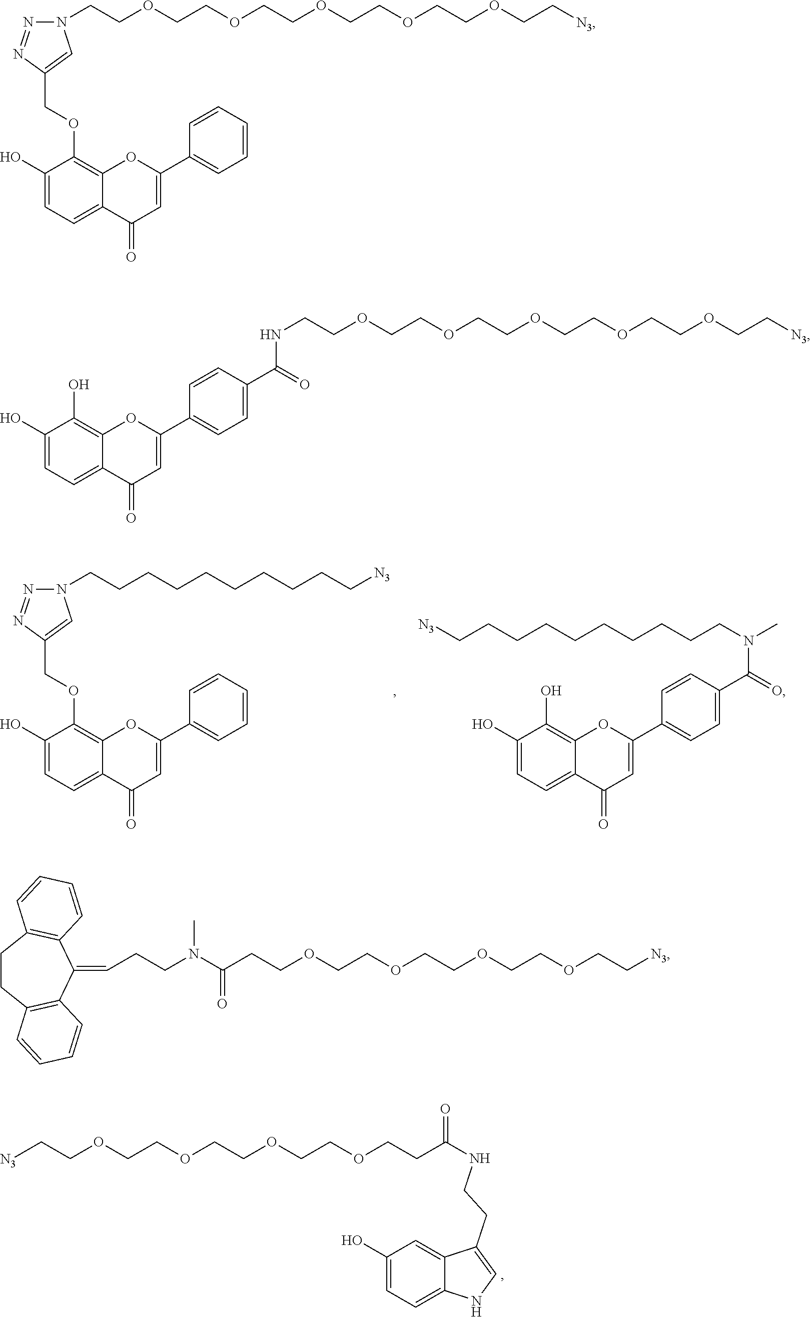 [SMILES: [N-]=[N+]=NCCOCCOCCOCCOCCOCCn1cc(COc2c(O)ccc3c(=O)cc(-c4ccccc4)oc23)nn1 ; [N-]=[N+]=NCCOCCOCCOCCOCCOCCNC(=O)c1ccc(-c2cc(=O)c3ccc(O)c(O)c3o2)cc1 ; [N-]=[N+]=NCCCCCCCCCCn1cc(COc2c(O)ccc3c(=O)cc(-c4ccccc4)oc23)nn1 ; CN(CCCCCCCCCCN=[N+]=[N-])C(=O)c1ccc(-c2cc(=O)c3ccc(O)c(O)c3o2)cc1 ; CN(CCC=C1c2ccccc2CCc2ccccc21)C(=O)CCOCCOCCOCCOCCN=[N+]=[N-] ; [N-]=[N+]=NCCOCCOCCOCCOCCC(=O)NCCc1c[nH]c2ccc(O)cc12]

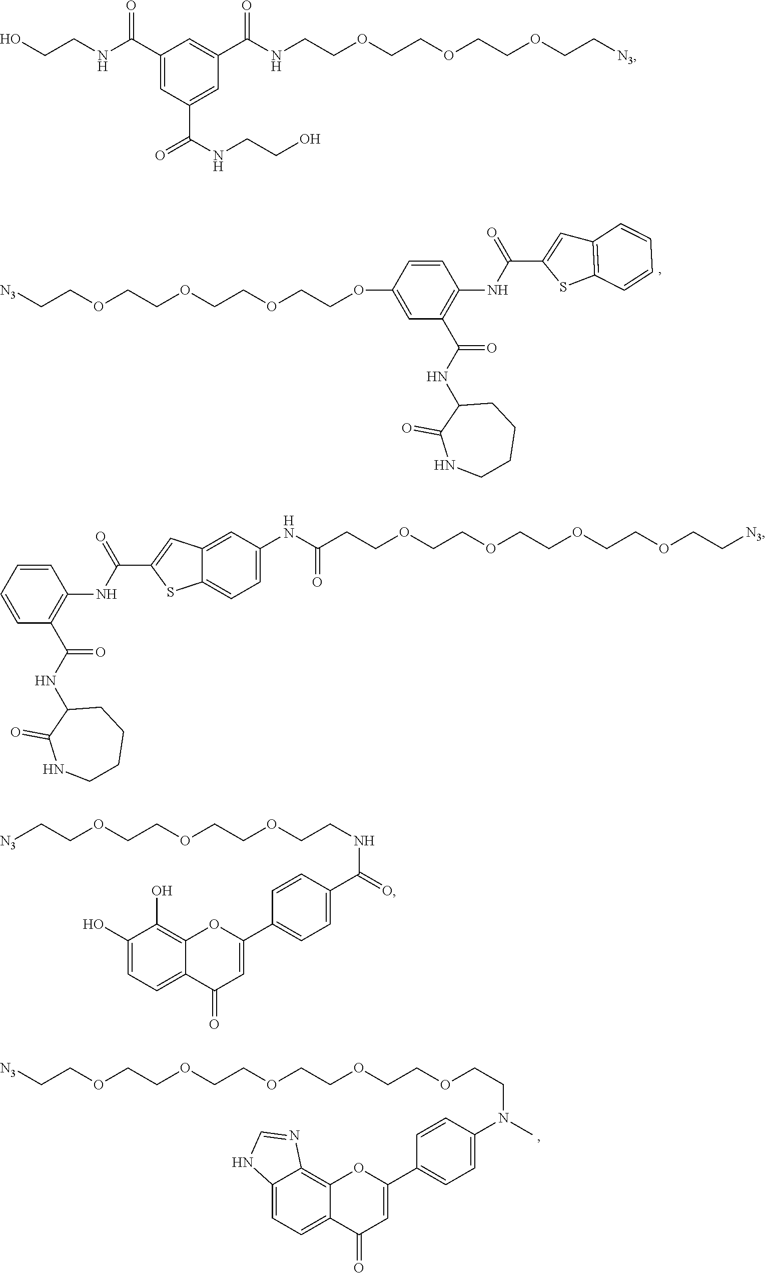

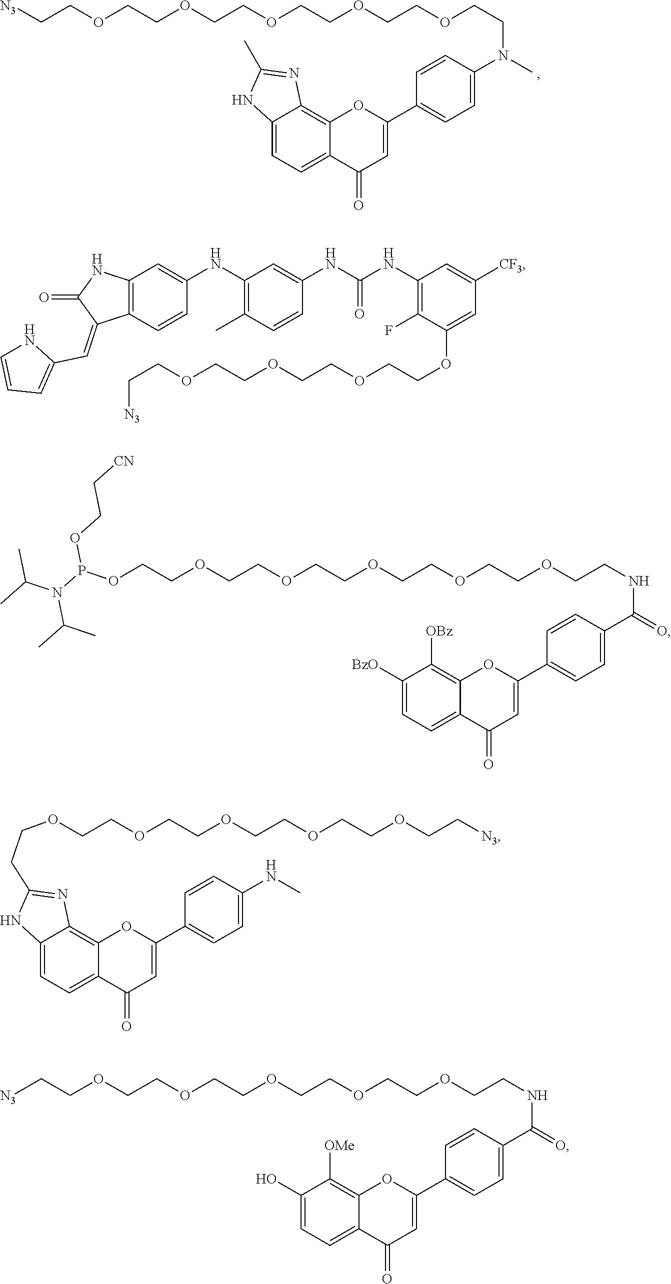

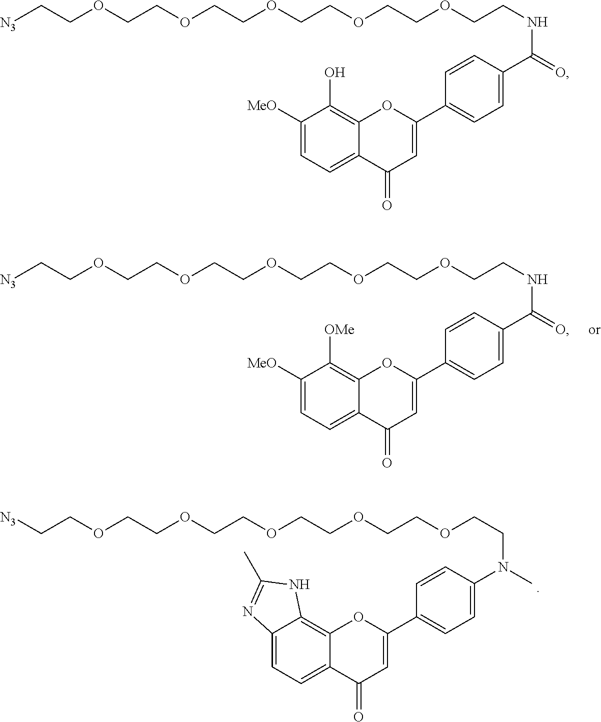 [SMILES: COc1ccc2c(=O)cc(-c3ccc(C(=O)NCCOCCOCCOCCOCCOCCN=[N+]=[N-])cc3)oc2c1O ; COc1ccc2c(=O)cc(-c3ccc(C(=O)NCCOCCOCCOCCOCCOCCN=[N+]=[N-])cc3)oc2c1OC ; Cc1nc2ccc3c(=O)cc(-c4ccc(N(C)CCOCCOCCOCCOCCOCCN=[N+]=[N-])cc4)oc3c2[nH]1]

Embodiment 130. A compound, or a stereoisomer, tautomer, prodrug, or salt thereof, wherein the compound comprises the structure:

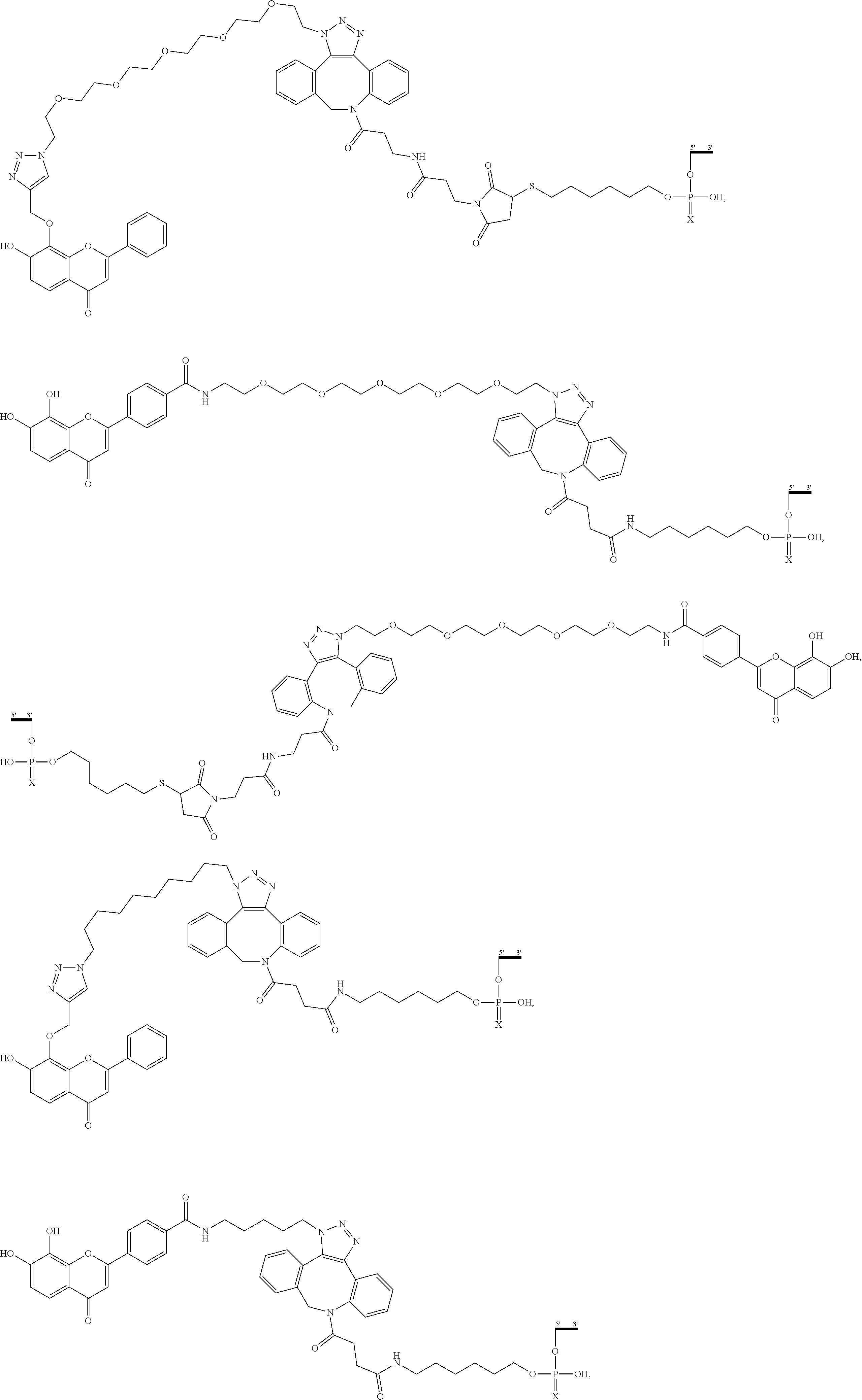

[chemical structure]

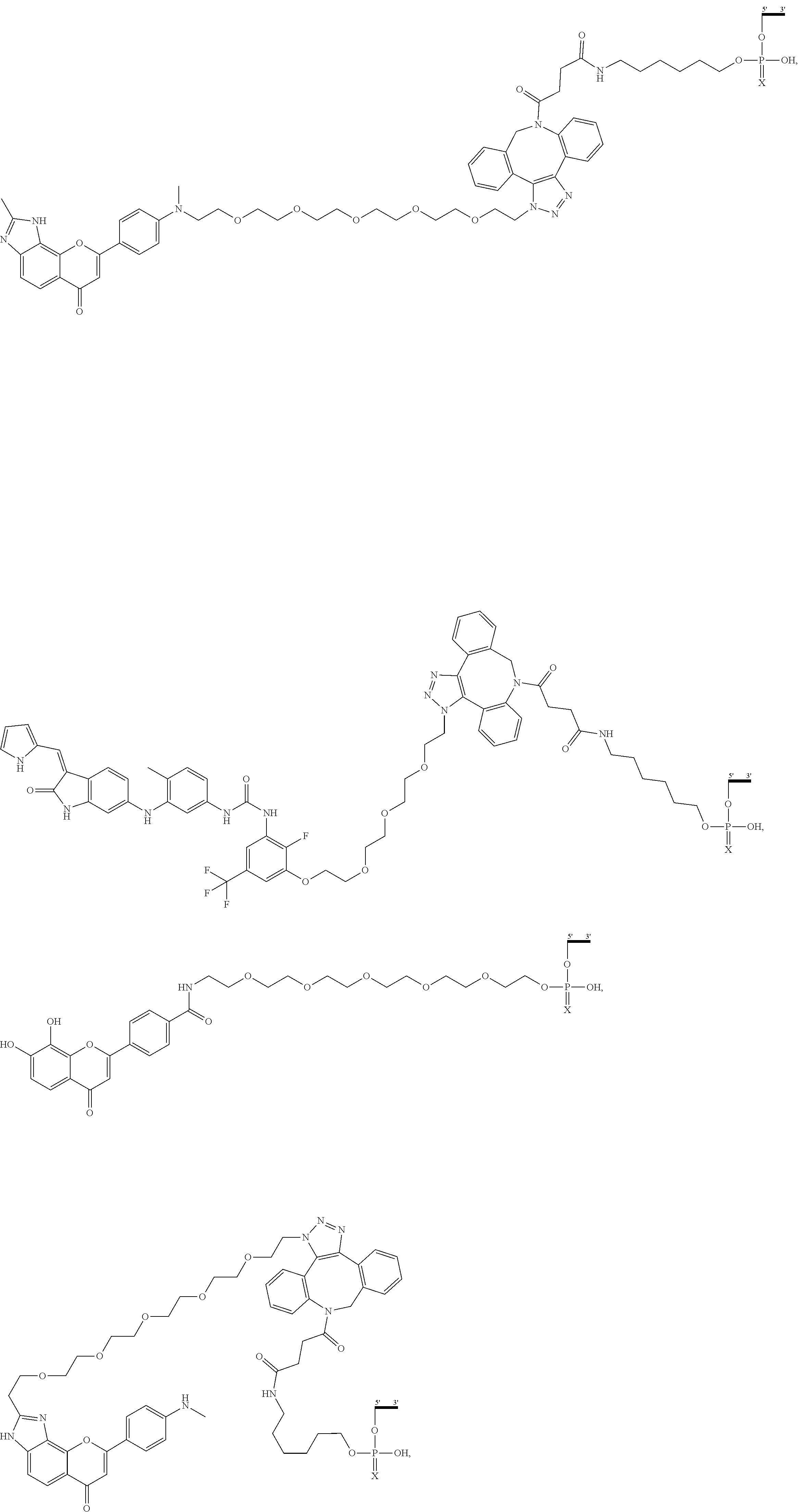

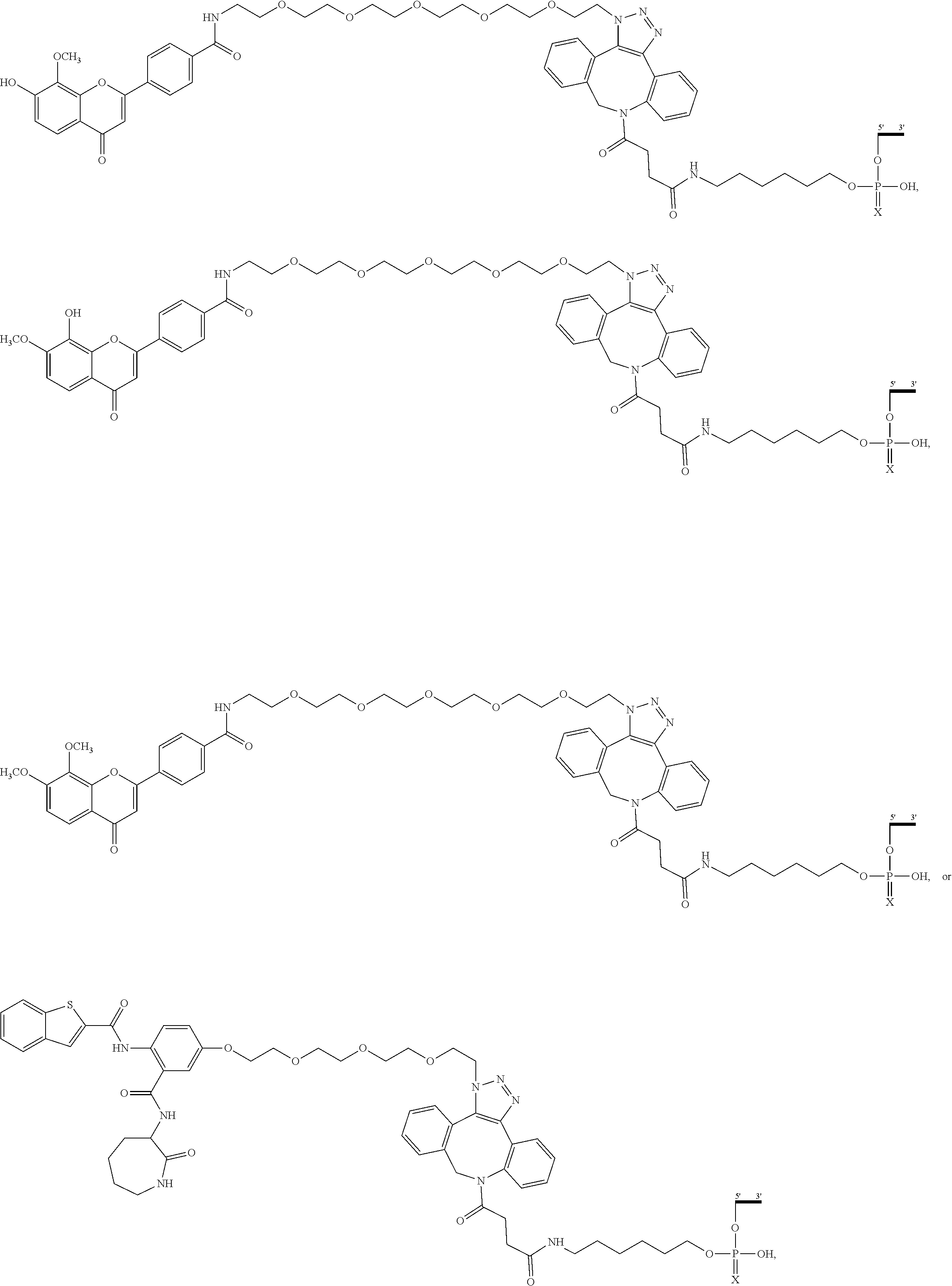

wherein:
[symbol] is an oligonucleotide; and
X is O or S.

Embodiment 131. A compound, or a stereoisomer, tautomer, prodrug, or salt thereof, wherein the compound comprises the structure:

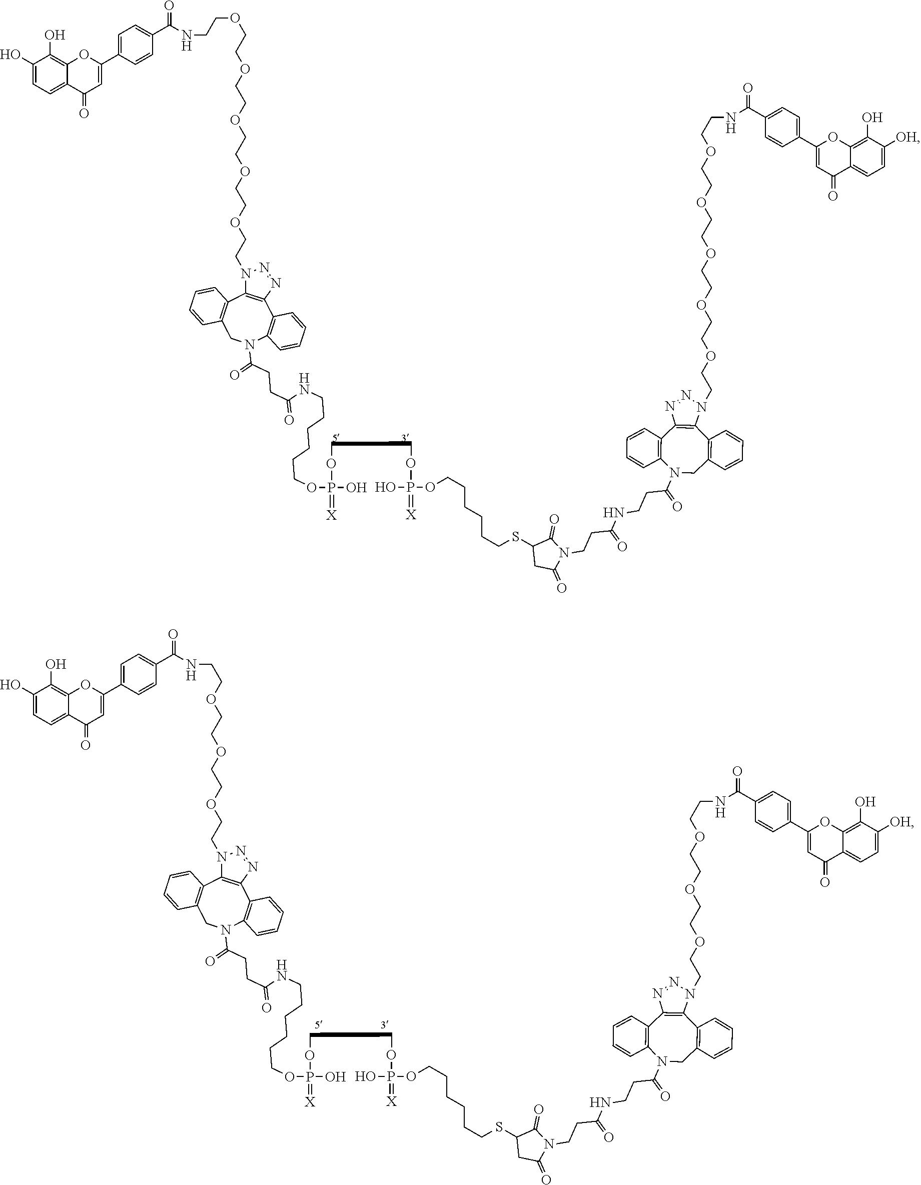

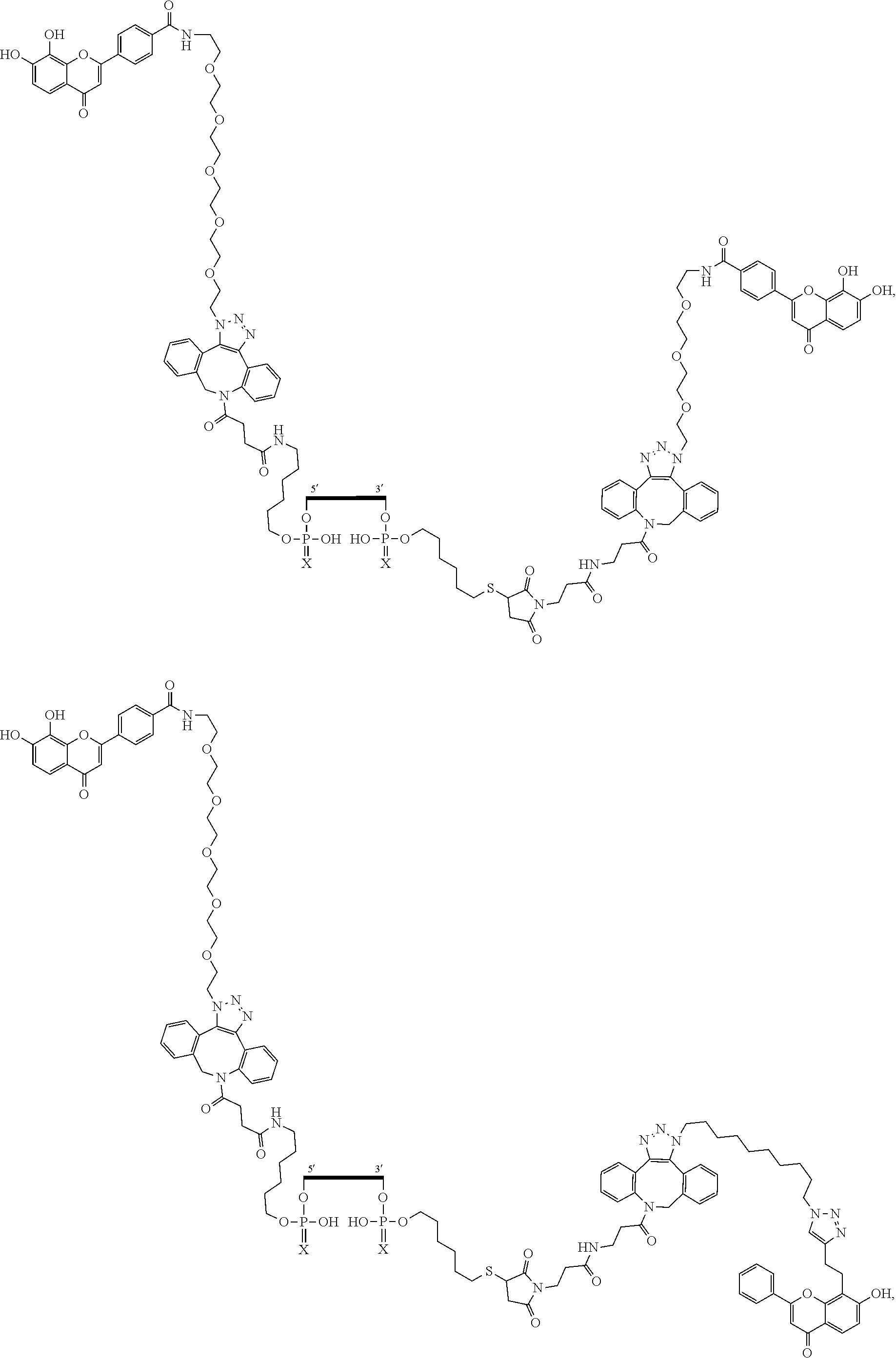

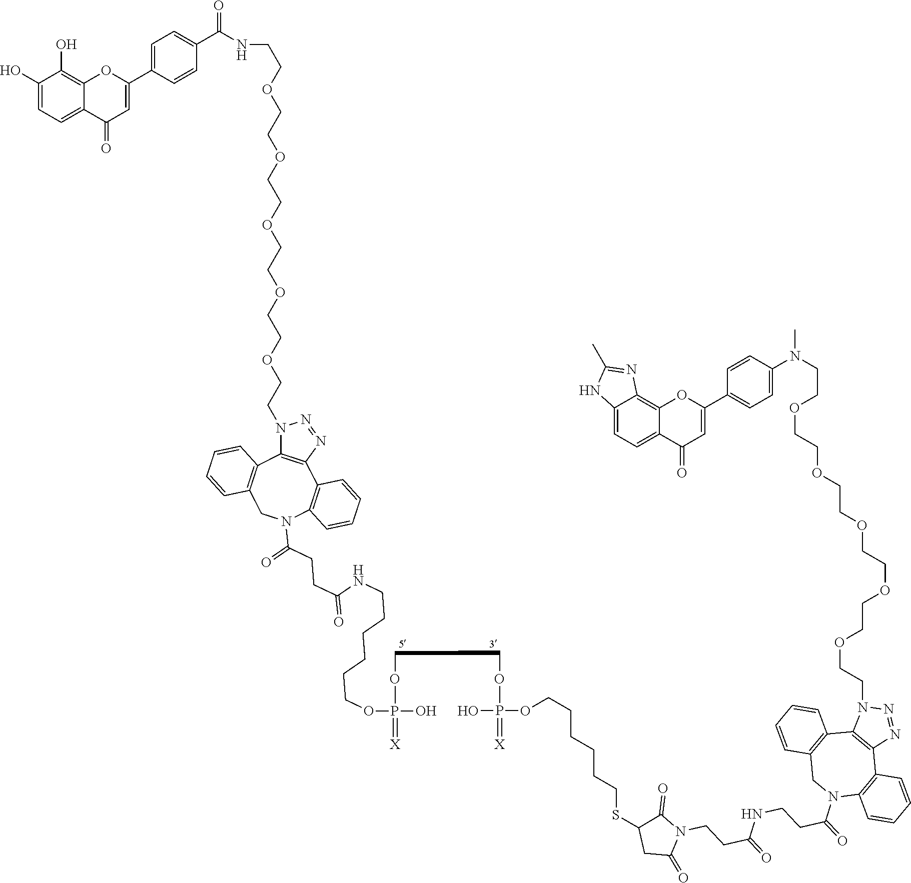

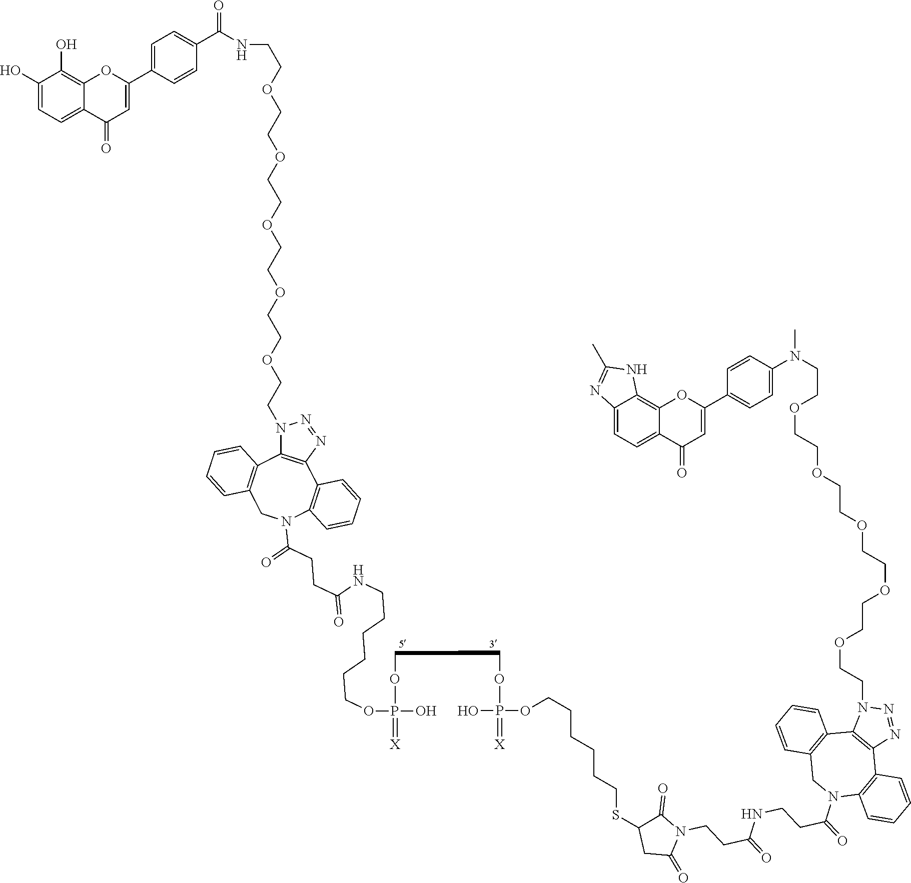

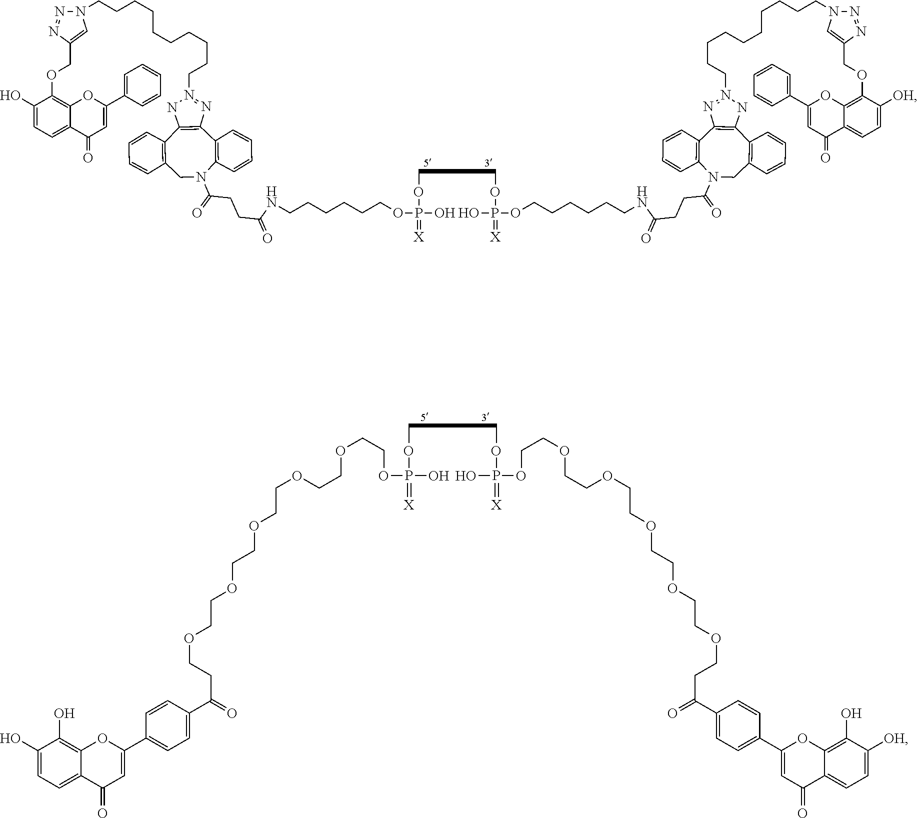

wherein:
[symbol] is an oligonucleotide; and
X is O or S.

Embodiment 132. The compound, or a stereoisomer, tautomer, prodrug, or salt thereof, of embodiment 130 or 131, wherein X is O.

Embodiment 132. The compound, or a stereoisomer, tautomer, prodrug, or salt thereof, of embodiment 130 or 131, wherein X is S.

Embodiment 133. The compound, or a stereoisomer, tautomer, prodrug, or salt thereof, of any one of embodiments 130-132, wherein the compound is in salt form.

Embodiment 134. The compound, or a stereoisomer, tautomer, prodrug, or salt thereof, of embodiment 133, wherein the salt is a sodium salt.

Embodiment 135. The compound, or a stereoisomer, tautomer, prodrug, or salt thereof, of embodiment 133, wherein the salt is a potassium salt.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

### EXAMPLES

In order that the embodiments described herein may be more fully understood, the following examples are set forth. The examples described in this application are offered to illustrate the compounds, compositions, and methods provided herein and are not to be construed in any way as limiting their scope.

### General Experimental Procedures

Definitions of variables in the structures in schemes herein are commensurate with those of corresponding positions in the formulae delineated herein.

### Common Abbreviations:

ACN acetonitrile
br broad
d doublet
DCM dichloromethane
dd doublet of doublets
dba dibenzylideneacetone
DBCO Azadibenzocyclooctyne
DFAA difluoroacetic anhydride
DIPEA diisopropylethylamine
DMF dimethylformamide
DMSO dimethyl sulfoxide
dppf 1,1′-ferrocenediyl-bis(diphenylphosphine)
EtOAc ethyl acetate
h hour(s)
HRMS high resolution mass spectrometry
HPLC high performance liquid chromatography
LCMS liquid chromatography and mass spectrometry
MS mass spectrometry
MW microwave
m multiplet
MeOH methanol
min minutes
mL milliliter(s)
MWCO molecular weight cut off
m/z mass to charge ratio
NMP N-methyl-2-pyrrolidone
NMR nuclear magnetic resonance
ppm parts per million
rt or RT room temperature
s singlet
t triplet
TFAA trifluoroacetic anhydride
TLC thin layer chromatography

### Example 1: Synthesis of TrkB Ligand-Conjugated Oligonucleotides

Exemplary compounds falling within the scope of the present disclosure can be synthesized according to the following procedures:

### General Procedure I:

To an aqueous solution of SC6 functionalized sense strand was added a solution of aqueous TCEP (50 eq, pH=7). Upon completion, the reaction was purified by HPLC. Pure fractions were collected and concentrated by MWCO. To the disulfide-reduced sense strand in RNAse free water was added DBCO-MAL (1)(5 eq) in CH₃CN. The reaction was monitored by LCMS and HPLC. Once the reaction was complete, the solid was filtered off and the solution was purified by HPLC and dried by lyophilization. The dried DBCO modified sense strand was reconstituted in RNase free water, and TrkB ligand 3 (2 eq) in THF was added. After the reaction was complete, the conjugate was purified by MWCO (5×).

### General Procedure II:

To an aqueous solution of AC6 functionalized sense strand was added a solution of DBCO-NHS (2)(3 eq) in CH₃CN. The reaction was monitored by LCMS and HPLC. Once the reaction was complete, the solid was filtered off, and the solution was purified by HPLC and dried by lyophilization. The dried DBCO modified sense strand was reconstituted in RNase free water and TrkB ligand 4 (2 eq) in THF was added. After the reaction was complete, the conjugate was purified by MWCO (5×).

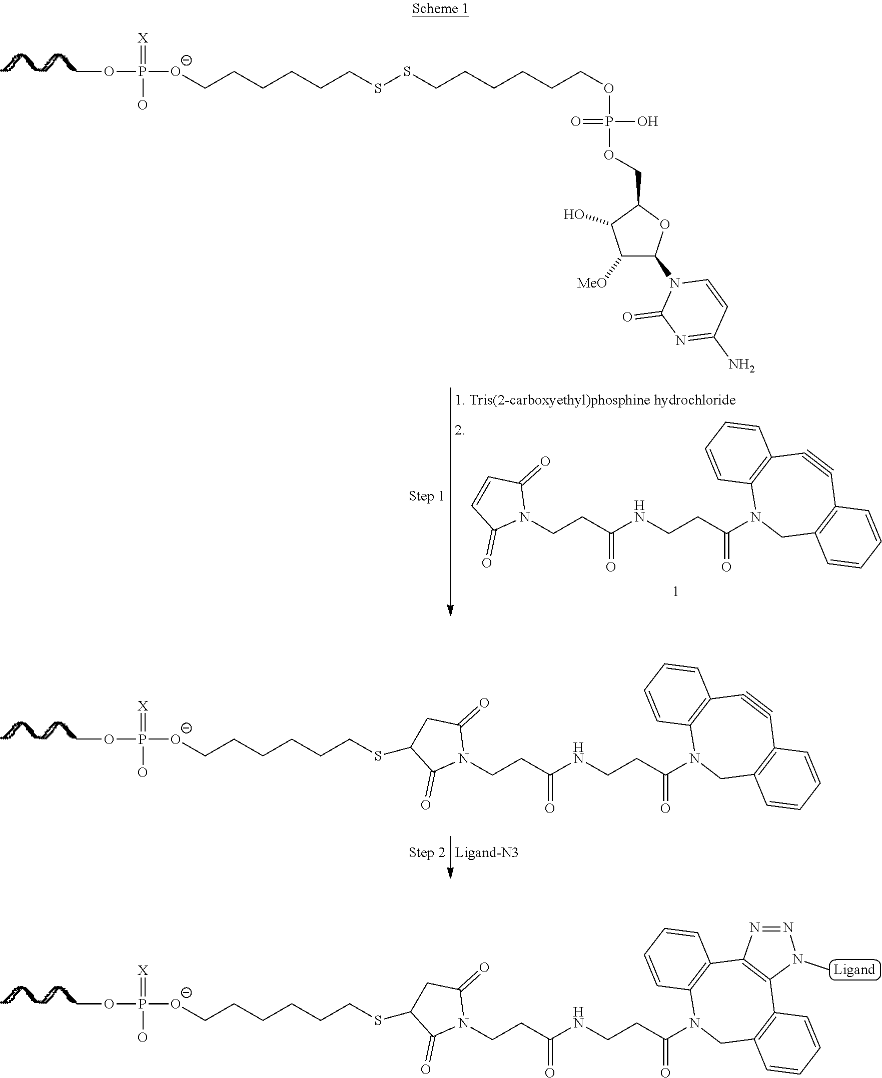

### General Procedure for Conjugation-Type I

Step 1. To a solution of 3′ (C6-SS—C6) or 5′ (C6-SS—C6) functionalized oligo sense strand (1 eq) in H₂O was added 10% V/V 1M sodium phosphate buffer (pH=7). Tris(2-carboxyethyl) phosphine hydrochloride (TCEP)(25 eq) was dissolved in H₂O and the pH was adjusted to 7 using 10 M NaOH. The TCEP solution was added to the solution of sense strand. The reaction is monitored by HPLC and LCMS. Upon completion, TCEP was removed by molecular weight cut-off with 100 mM sodium phosphate buffer (pH=7) using Amicon® Ultra-15 Centrifugal (3K, 3 times). Then DBCO-MAL (1)(3-5 eq) in DMSO or CH₃CN was added to the solution of disulfide-reduced sense strand. The reaction is monitored by HPLC and LCMS. Upon completion, the 3′ DBCO functionalized oligo sense strand was purified by reverse phase HPLC. The product fractions were collected and lyophilized to afford a white solid. The solid was reconstituted in H₂O for the next step.

Step 2. To a solution of 3′ DBCO functionalized oligo sense strand (1 eq) was added a solution of ligand-N₃ (1.5 eq) in THF or DMSO. The reaction is monitored by HPLC and LCMS. Upon completion, the conjugate was purified by reverse phase HPLC or molecular weight cut-off with Amicon® Ultra-15 Centrifugal filter (3K, 5 times).

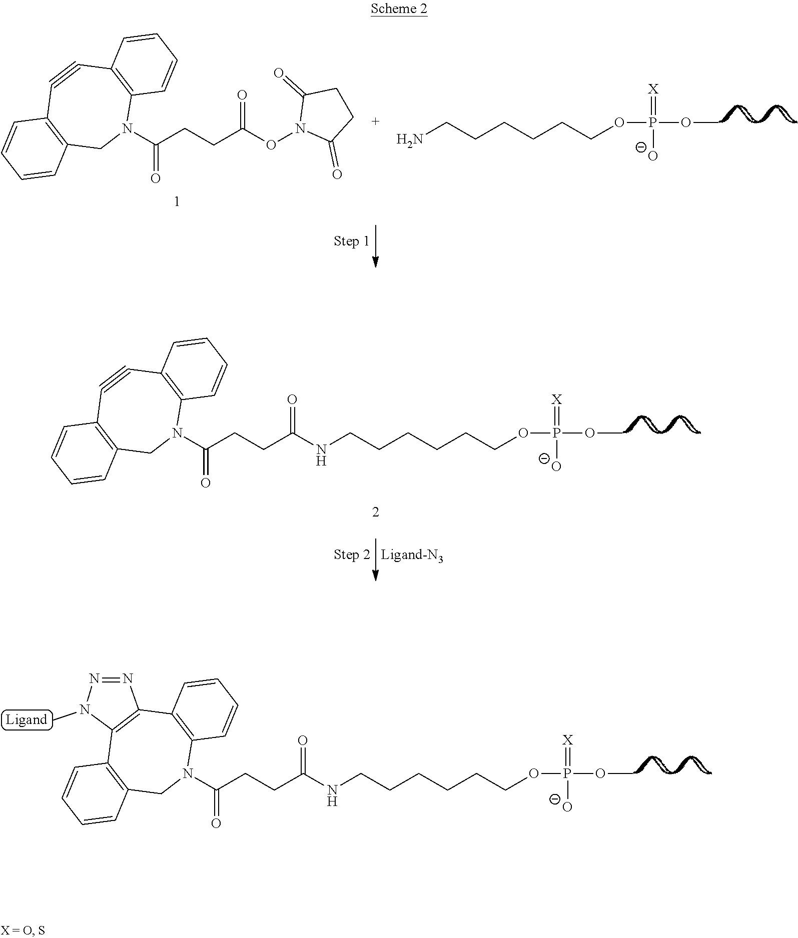

### General Procedure for Conjugation-Type II:

Step 1. To a solution of 5′ amine functionalized oligo sense strand (1 eq) in H₂O was added 10% V/V 1M sodium phosphate buffer (pH=7) and 20%-50% V/V CH₃CN. A solution of DBCO-NHS (1)(1.5-3 eq) in DMSO or CH₃CN was added to the reaction. The reaction is monitored by HPLC and LCMS. Upon completion, the reaction was purified by reverse phase HPLC. The product fractions were collected and lyophilized to afford a white solid. The solid was reconstituted in H₂O for the next step.

Step 2. To a solution of 5′ DBCO functionalized oligo sense strand 2 (1 eq) was added a solution of ligand-N₃ (1.5 eq) in THF or DMSO. The reaction is monitored by HPLC and LCMS. Upon completion, the reaction was purified by reverse phase HPLC or molecular weight cut-off with Amicon® Ultra-15 Centrifugal filter (3K, 5 times).

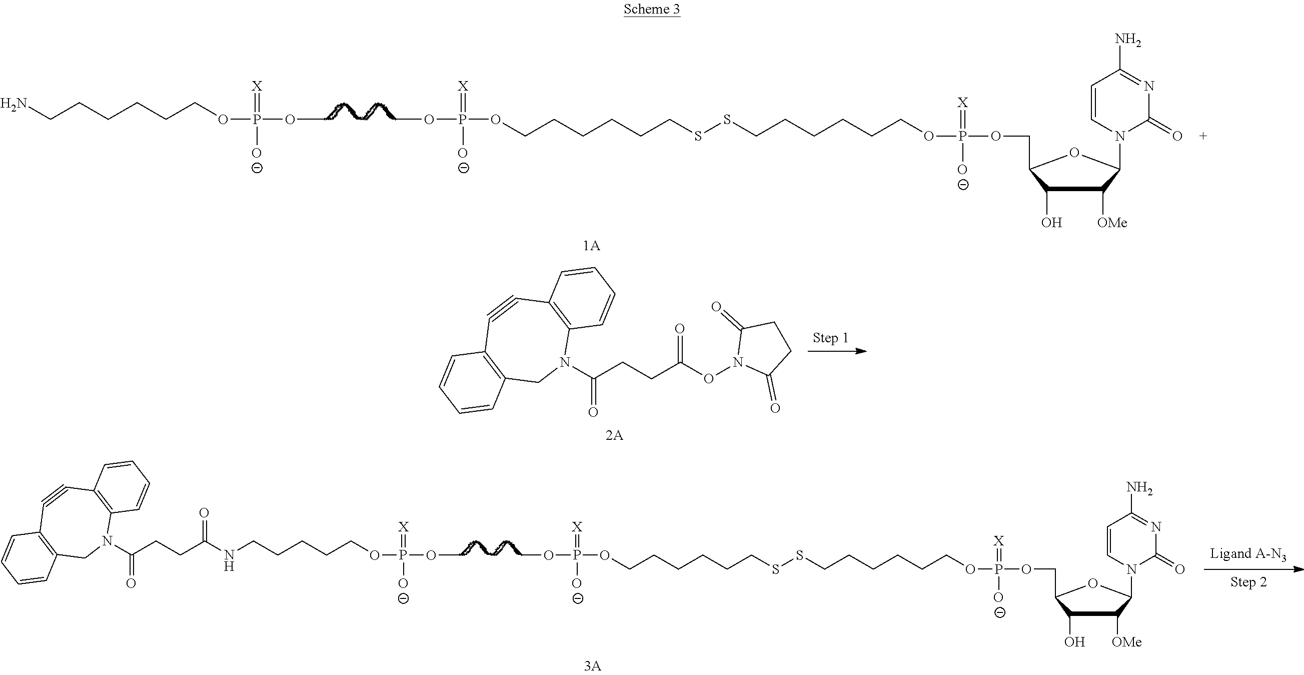

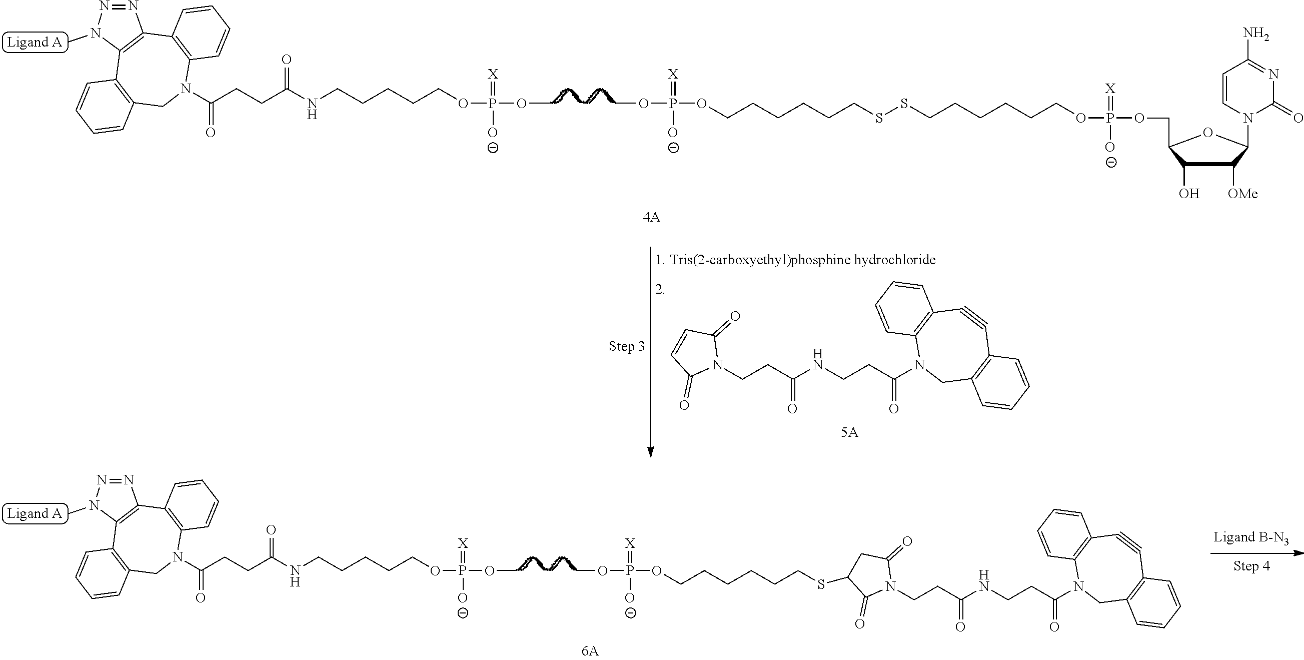

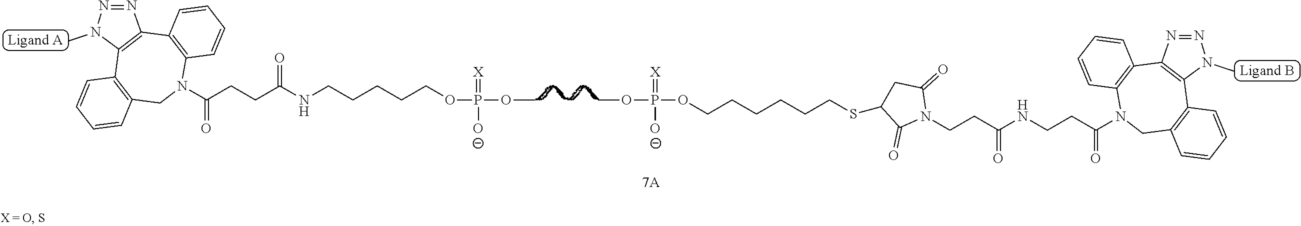

### General Procedure a for Conjugation-Type III:

Step 1. To a solution of 5′ amine and 3′ (C6-SS—C6) functionalized oligo sense strand 1A (1 eq) in H₂O was added 10% V/V 1M sodium phosphate buffer (pH=7) and 20%-50% V/V CH₃CN. A solution of DBCO-NHS (2A)(1.5-3 eq) in DMSO or CH₃CN was added to the reaction. The reaction is monitored by HPLC and LCMS. Upon completion, the reaction was purified by reverse phase HPLC. The product fractions were collected and lyophilized to afford a white solid. The solid was reconstituted in H₂O for the next step.

Step 2. To a solution of 5′ DBCO functionalized oligo sense strand 3A (1 eq) was added a solution of ligand-A-N₃ (1.5 eq) in THF or DMSO. The reaction is monitored by HPLC and LCMS. Upon completion, the reaction was purified by reverse phase HPLC or molecular weight cut-off with Amicon® Ultra-15 Centrifugal filter (3K, 3 times).

Step 3. To a solution of oligo sense strand 4A (1 eq) in H₂O was added 10% V/V 1M sodium phosphate buffer (pH=7). Tris(2-carboxyethyl) phosphine hydrochloride (TCEP)(25 eq) was dissolved in H₂O and the pH was adjusted to 7 using 10 M NaOH. The TCEP solution was added to the solution of sense strand. The reaction is monitored by HPLC and LCMS. Upon completion, TCEP was removed by molecular weight cut-off with 100 mM sodium phosphate buffer (PH=7) using Amicon® Ultra-15 Centrifugal filter (3K, 3 times). Then DBCO-MAL (5A) (3-5 eq) in DMSO or CH₃CN was added to the solution of disulfide-reduced sense strand. The reaction is monitored by HPLC and LCMS. Upon completion, the 3′ DBCO functionalized oligo sense strand 6A was purified by reverse phase HPLC. The product fractions were collected and lyophilized to afford a white solid. The solid was reconstituted in H₂O for the next step.

Step 4. To a solution of 3′ DBCO functionalized oligo sense strand (6A)(1 eq) was added a solution of ligand-B—N₃ (1.5 eq) in THF or DMSO. The reaction is monitored by HPLC and LCMS. Upon completion, the conjugate was purified by reverse phase HPLC or molecular weight cut-off with Amicon® Ultra-15 Centrifugal filter (3K, 5 times).

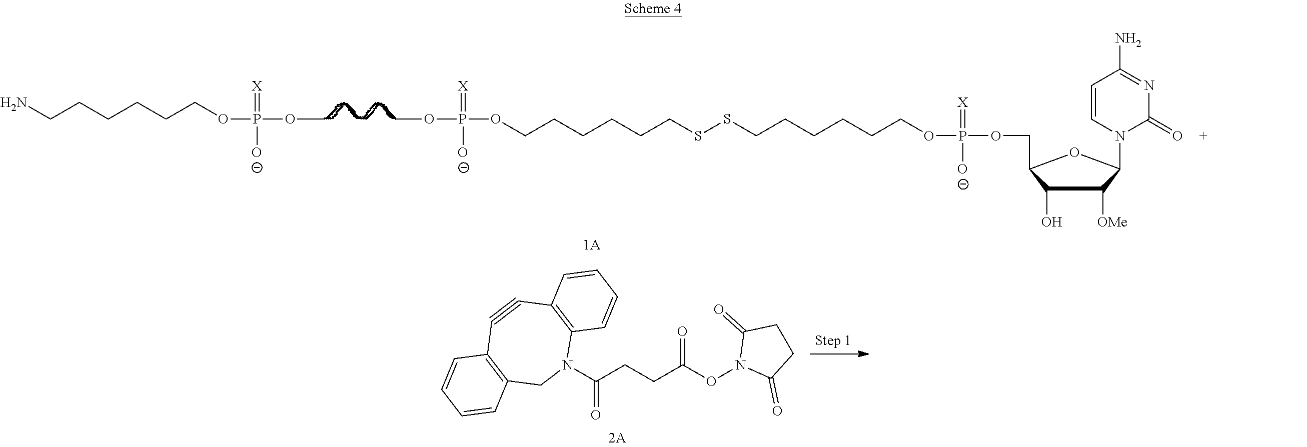

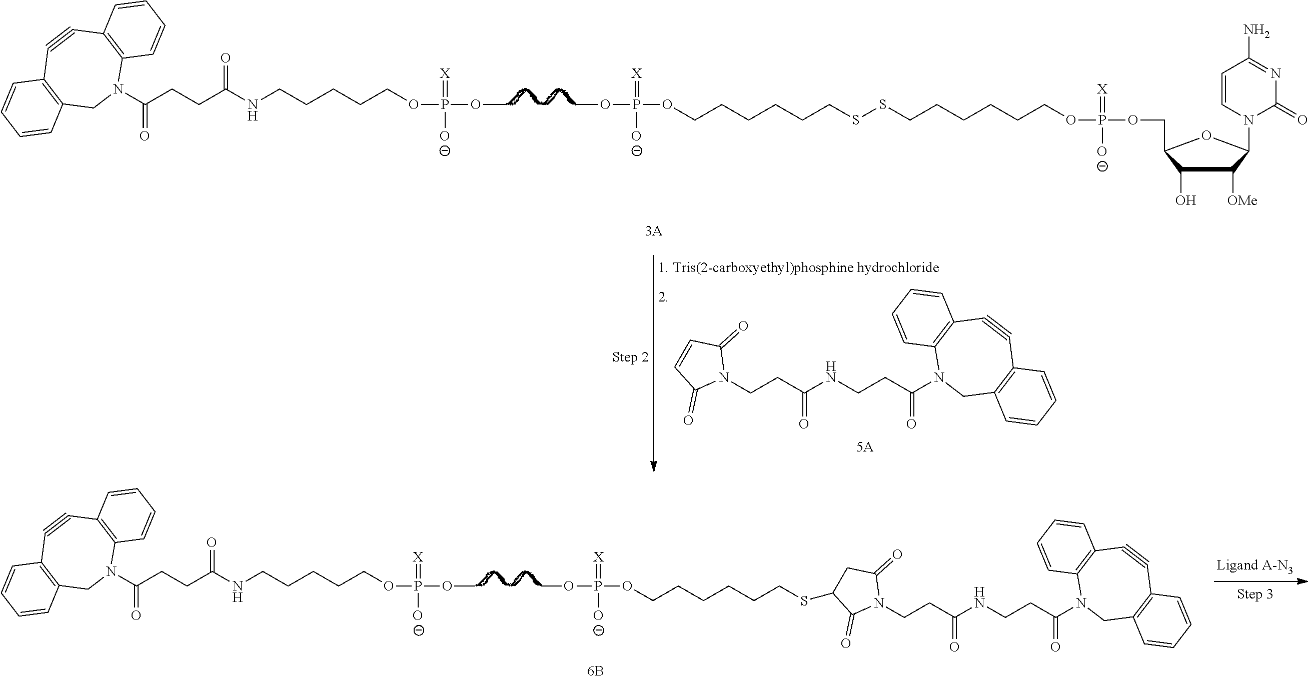

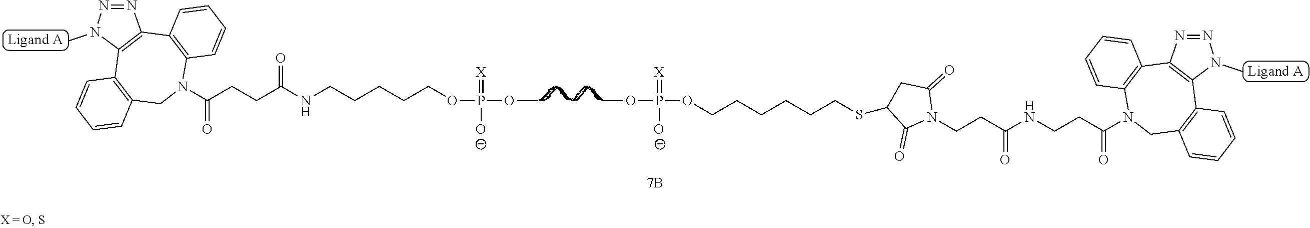

### General Procedure B for Conjugation-Type III:

Step 1. To a solution of 5′ amine and 3′ (C6-SS—C6) functionalized oligo sense strand 1A (1 eq) in H₂O was added 10% V/V 1M sodium phosphate buffer (pH=7) and 20%-50% V/V CH₃CN. A solution of DBCO-NHS (2)(1.5-3 eq) in DMSO or CH₃CN was added to the reaction. The reaction is monitored by HPLC and LCMS. Upon completion, the solids were removed by centrifuge. Then the reaction was lyophilized to afford a white solid. The solid was reconstituted in H₂O and dialyzed against water (1×) using Amicon® Ultra-15 Centrifugal filter (3K).

Step 2. To a solution of 5′ DBCO functionalized oligo sense strand 3A (1 eq) in H₂O was added 10% V/V 1M sodium phosphate buffer (pH=7). Tris(2-carboxyethyl) phosphine hydrochloride (TCEP)(25 eq) was dissolved in H₂O and the pH was adjusted to 7 using 10 M NaOH. The TCEP solution was added to the solution of sense strand. The reaction is monitored by HPLC and LCMS. Upon completion, TCEP was removed by molecular weight cut-off with 100 mM sodium phosphate buffer (PH=7) using Amicon® Ultra-15 Centrifugal filter (3K, 3 times). Then DBCO-MAL (5)(3-5 eq) in DMSO or CH₃CN was added to the solution of disulfide-reduced sense strand. The reaction is monitored by HPLC and LCMS. Upon completion, the 3′ and 5′ DBCO functionalized oligo sense strand 5 was purified by reverse phase HPLC. The product fractions were collected and lyophilized to afford a white solid. The solid was reconstituted in H₂O for the next step.

Step 3. To a solution of 3′ and 5′ DBCO functionalized oligo sense strand 6B (1 eq) was added a solution of ligand A-N₃ (3 eq) in THF or DMSO. The reaction is monitored by HPLC and LCMS. Upon completion, the conjugate was purified by reverse phase HPLC or molecular weight cut-off with Amicon® Ultra-15 Centrifugal filter (3K, 5 times).

### General Procedure for Annealing:

The concentrations of both sense strand and antisense strand were determined by Nanodrop. The double-stranded siRNA was prepared by mixing equimolar of sense stand and antisense strand. The annealing process was monitored by RP-HPLC, non-denaturing method. After annealing, no more that 5% of antisense strand was in the duplex mixture. Duplex concentration was determined by measuring the solution absorbance on Nanodrop.

### Example 2: BA-117 Conjugate

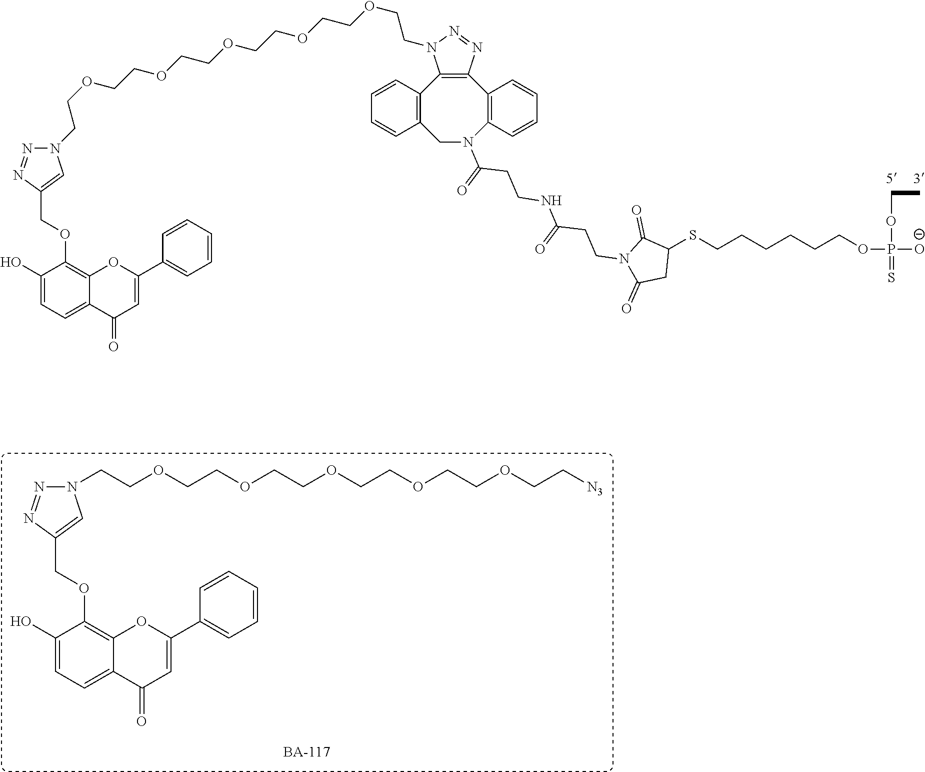

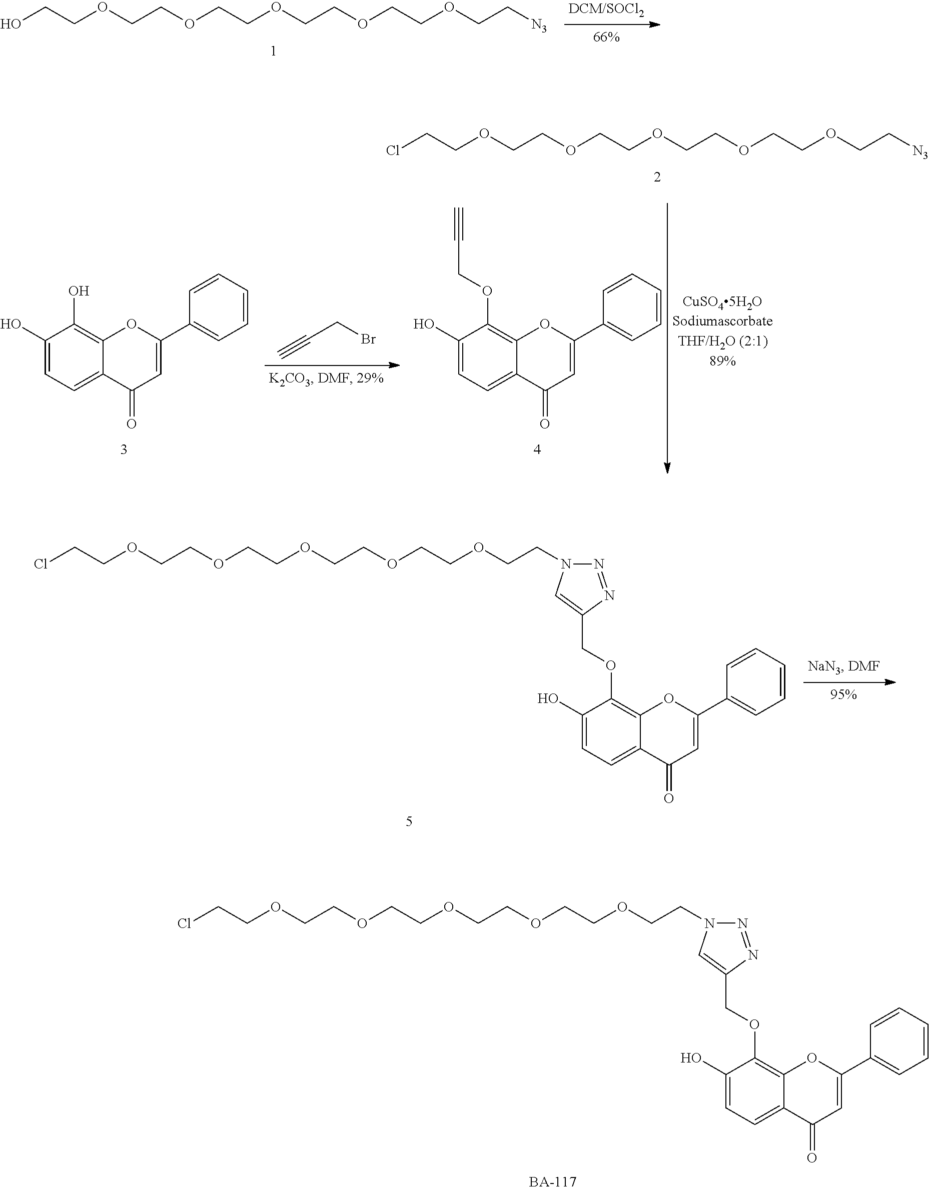

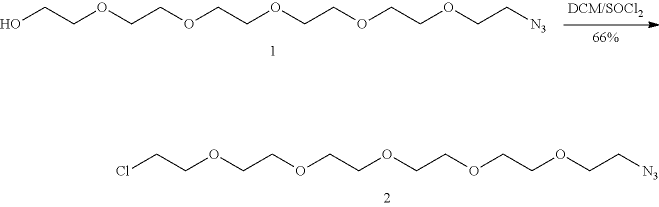 [SMILES: [N-]=[N+]=NCCOCCOCCOCCOCCOCCO ; [N-]=[N+]=NCCOCCOCCOCCOCCOCCCl]

To a stirred solution of azido alcohol 1 (0.500 g, 0.780 mmol, 1 eq) in DCM (5 mL), was added thionyl chloride (1.1 mL 15.60 mmol, 20 eq) dropwise. The reaction mixture was stirred at 50° C. for 12 h. Reaction mixture was diluted with DCM 50 mL, washed with saturated NaHCO₃20 mL, and the organic layer was dried and purified by flash chromatography to obtain chloro azide 2 (500 mg, 66%) as a gum NMR and LCMS (m/z=348, M+Na) are corresponding with product.

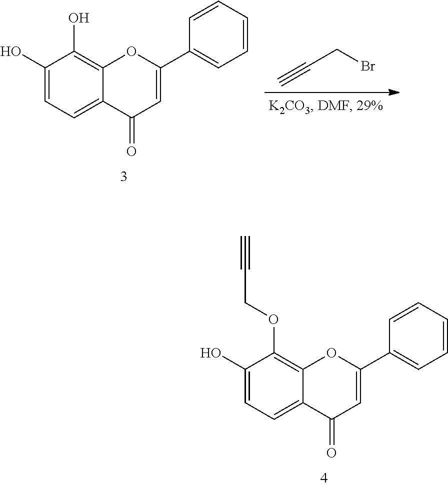

To a stirred suspension of dihydroxy flavone 3 (1.5 g, 5.906 mmol, 1 eq) and K₂CO₃ (0.815 g, 5.906 mmol, 1 eq) in DMF (20 mL) was added Propargyl bromide (0.447 mL, 5.906 mmol, 1 eq), the mixture was stirred overnight at RT. Water (100 mL) was added to the reaction mixture, resulting precipitate di-propargyl was filtered off, the filtrate was extracted with DCM 2×100 mL, dried over Na₂SO₄, concentrated, resulting solid was triturated with MeOH, filtered, and dried under high vacuum to obtain mono propargyl flavanol 4 (500 mg, 29%) as a white solid. NMR and LCMS (m/z=293 M+1) are corresponding with product. ¹H NMR (499 MHz, DMSO-d₆) δ 10.85 (s, 1H), 8.17-8.09 (m, 2H), 7.68 (d, J=8.8 Hz, 1H), 7.65-7.55 (m, 3H), 7.03 (d, J=8.8 Hz, 1H), 6.94 (s, 1H), 4.90 (d, J=2.5 Hz, 2H), 3.54 (t, J=2.4 Hz, 1H). ¹H NMR (499 MHz, CD₃OD) δ 8.15-8.07 (m, 2H), 7.80 (d, J=8.9 Hz, 1H), 7.63-7.53 (m, 3H), 7.03 (d, J=8.9 Hz, 1H), 6.85 (s, 1H), 4.95 (d, J=2.5 Hz, 2H), 2.91 (t, J=2.4 Hz, 1H).

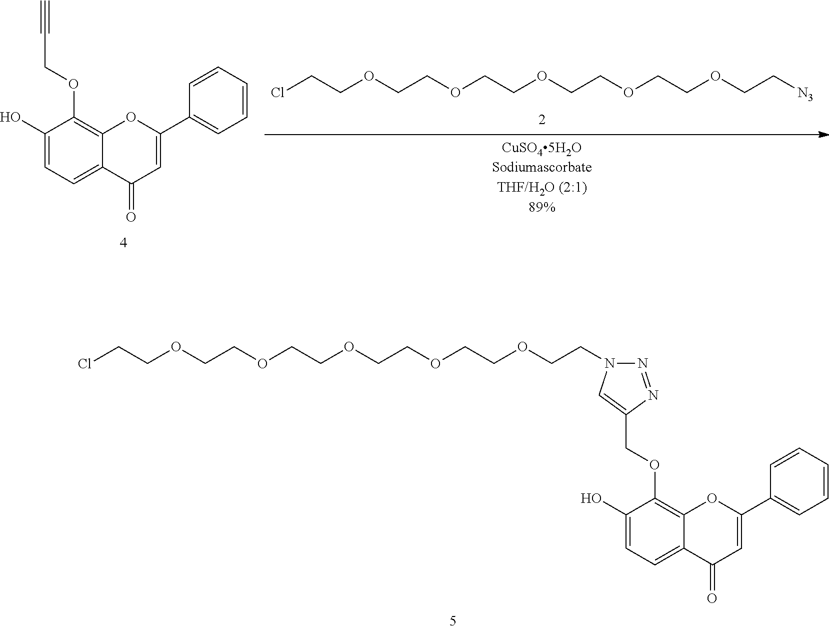

To a solution of alkyne 4 (0.290 g, 0.993 mmol, 1 eq) and azide 2 (0.323 g, 0.993 mmol, 1 eq) in THF (10 mL) at RT was added a solution of CuSO₄·5H₂O (50 mg, 0.2 mmol, 0.2 eq) in water (1 mL) followed by a solution of sodium ascorbate (60 mg, 0.3 mmol, 0.3 eq) in water (1 mL). The reaction was stirred for 2 hrs at RT. Then the reaction was diluted with aq. saturated NaHCO₃ (50 mL) The aqueous phase was extracted with DCM (100 mL). The organic layers were combined, dried over Na₂SO₄, filtered, and concentrated. The residue was purified by 0-10% MeOH in DCM (25 g, 20 μM, in 15 CV) to give 550 mg of triazole 5 (89%) as a gum. NMR and mass m/z=642 (M+1) are corresponding with product.

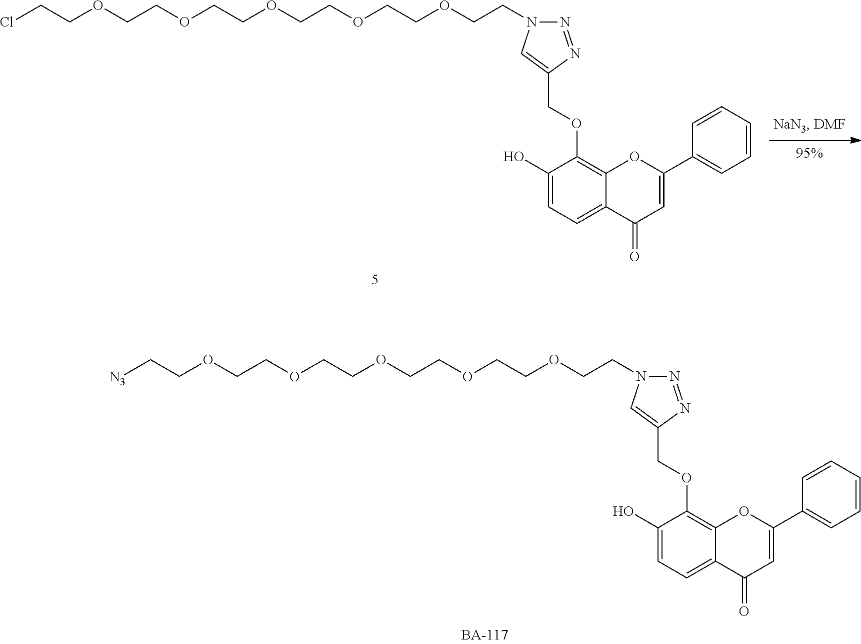 [SMILES: O=c1cc(-c2ccccc2)oc2c(OCc3cn(CCOCCOCCOCCOCCOCCCl)nn3)c(O)ccc12 ; [N-]=[N+]=NCCOCCOCCOCCOCCOCCn1cc(COc2c(O)ccc3c(=O)cc(-c4ccccc4)oc23)nn1]

A solution of Chloro derivative 5 (0.270 g, 0.437 mmol, 1 eq) and sodium azide (74 mg, 2.184 mmol, 5 eq) in dry DMF (5 mL) under argon was heated to 60° C. for 12 h. LCMS showed complete conversion. The reaction was cooled to room temperature, diluted with aq. saturated bicarbonate (50 mL) and extracted with DCM (3×100 mL). The combined organic layers were washed with brine (100 mL), dried over Na₂SO₄, and evaporated. The residue was purified by silica gel chromatography, eluting with 0-10% MeOH/DCM to afford the azide BA-117 as a yellow syrup (260 mg, 95%) confirmed by NMR and LCMS m/z 625 (M+1). 99% HPLC purity. ¹H NMR (499 MHz, DMSO-d₆) δ 8.13 (s, 1H), 8.03-7.96 (m, 2H), 7.65 (d, J=8.8 Hz, 1H), 7.62-7.52 (m, 3H), 7.04 (d, J=8.8 Hz, 1H), 6.90 (s, 1H), 5.25 (s, 2H), 4.42 (t, J=5.3 Hz, 2H), 3.69 (t, J=5.3 Hz, 2H), 3.59-3.54 (m, 2H), 3.54-3.39 (m, 16H), 3.37 (dd, J=5.5, 4.3 Hz, 3H).

BA-117 azide was conjugated to an oligo sense strand according to general procedure type I. MW calculated: 8436.81. MW Found: 8436.67.

### Example 3: BA-118 Conjugate

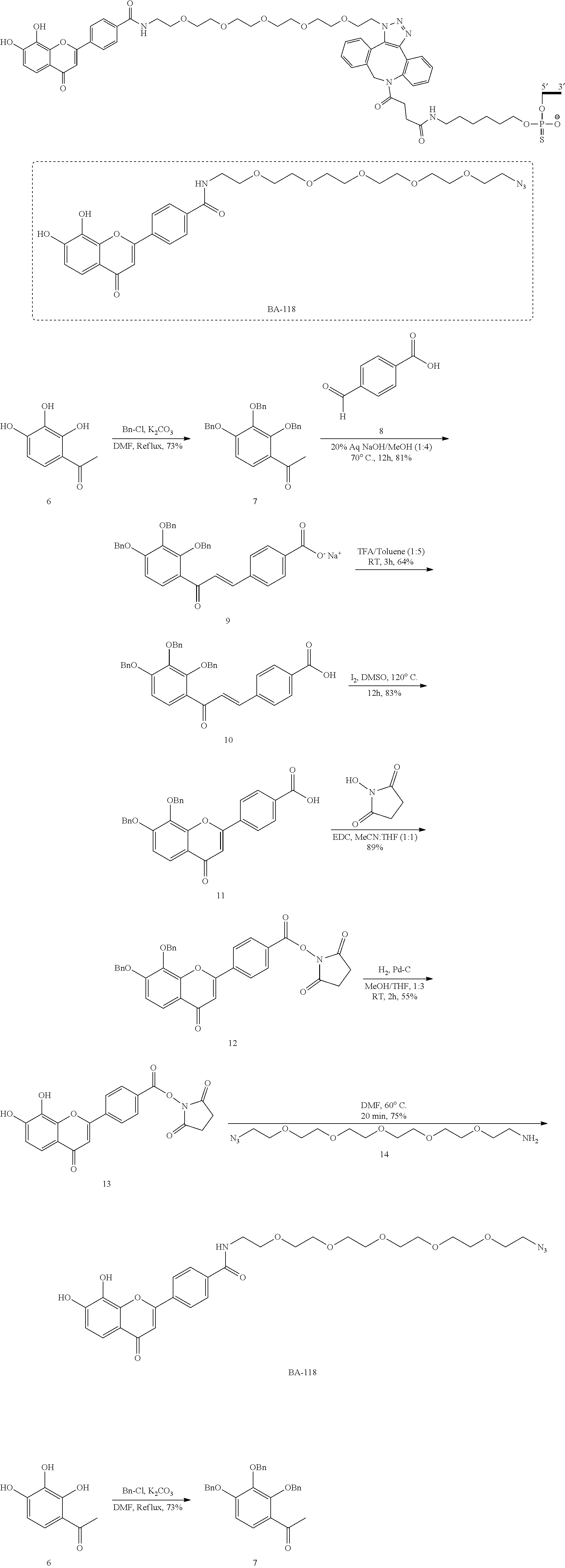

A mixture of 2′,3′,4′-trihydroxyacetophenone 6 (2 g, 11.905 mmol, 1 eq), benzyl chloride (6.818 mL, 59.524 mmol, 5 eq) and anhydrous K₂CO₃ (6.57 g, 47.619 mmol, 4 eq) in DMF (10 mL) was refluxed under N₂ for 3 h. The solution was then poured into ice and the resulting precipitate was collected by filtration, washed with water, dried under high vacuum to obtain benzyl ether 7 (3.8 g, 73%) as a white solid. LCMS m/z=439 M+1, and NMR are corresponding with product.

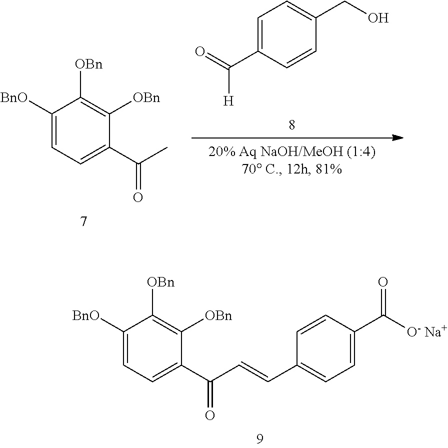

To a stirred solution of Acetophenone 7 (3.5 g 7.991 mmol, 1 eq), Aldehyde 8 (1.558 g, 10.388 mmol, 1.3 eq) in MeOH (20 mL) was added 20% NaOH (10 mL). The suspension was stirred at 70° C. for 5 h. Reaction mixture was cooled, diluted with water precipitated solid was filtered and dried under high vacuum to obtain chalcone 9 (3.7 g 81%) as a beige solid, used as it is for next step. LCMS m/z=570 M+1 and NMR are corresponding with product.

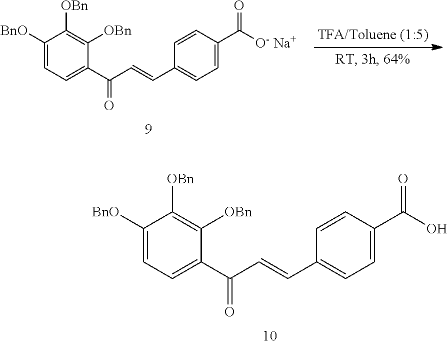

To a suspension of benzyl ether 9 (2 g, 3.378 mmol, 1 eq) in Toluene (20 mL) was added TFA (4 mL) After stirring for 2 hours, the solvent was evaporated, and the product was purified by flash column chromatography (0-10% methanol in dichloromethane), gave 1.08 g (64%) of alcohol 10 as a white solid. LCMS and NMR are corresponding with product.

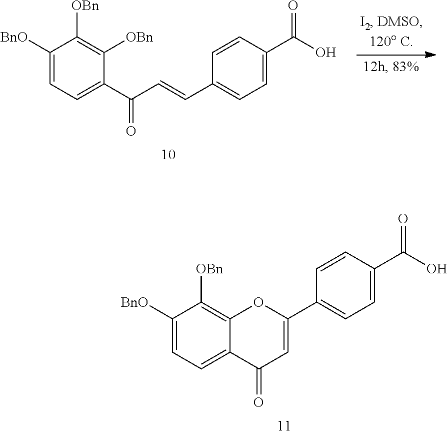 [SMILES: O=C(O)c1ccc(/C=C/C(=O)c2ccc(OCc3ccccc3)c(OCc3ccccc3)c2OCc2ccccc2)cc1 ; O=C(O)c1ccc(-c2cc(=O)c3ccc(OCc4ccccc4)c(OCc4ccccc4)c3o2)cc1]

To a solution of Chalcone 10 (1.08 g, 2.25 mmol, 1 eq) in 10 mL of DMSO was added iodine (29 mg, 0.225 mmol, 0.1 eq), and the mixture was heated to 120° C. After stirring for 12 h, the reaction mixture was cooled to rt and quenched with ice cold water. A slightly yellow precipitate appeared, that was filtered, washed with saturated Na₂S₂O₃, water and dried under high vacuum to obtain Flavone 11 (0.9 g, 83%) as a beige solid. LCMS and NMR are corresponding with product.

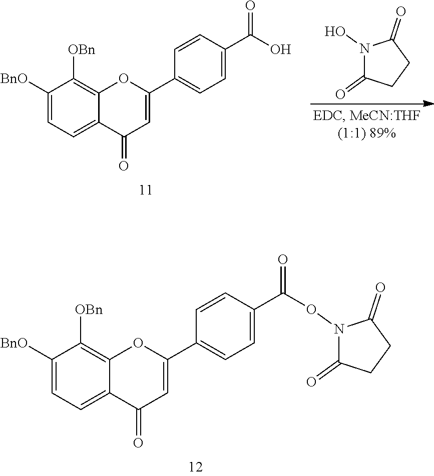 [SMILES: O=C(O)c1ccc(-c2cc(=O)c3ccc(OCc4ccccc4)c(OCc4ccccc4)c3o2)cc1 ; O=C1CCC(=O)N1O ; O=C(ON1C(=O)CCC1=O)c1ccc(-c2cc(=O)c3ccc(OCc4ccccc4)c(OCc4ccccc4)c3o2)cc1]

To a stirred solution of acid 11 (400 mg, 0.549 mmol), EDC·HCl (157 mg. 0.824 mmol, 1.5 eq), and N-hydroxysuccinimide (126 mg 1.1 mmol, 2 equiv) in 100 mL of anhydrous THF/MeCN (1:1) were heated to 40° C. and stirred overnight. The solvent was removed, and the residue was washed with isopropyl alcohol and ether and dried under high vacuum, to obtain 430 mg, 89% of the ester 12. LCMS M/z=576 M+1 and NMR are corresponding with product.

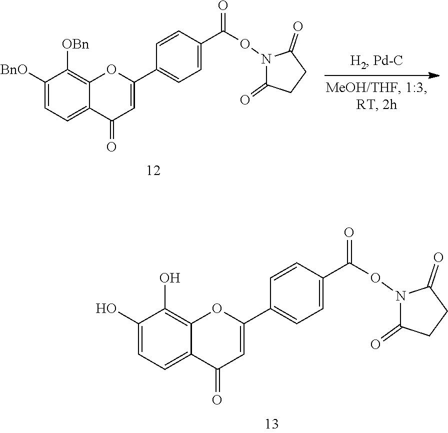 [SMILES: O=C(ON1C(=O)CCC1=O)c1ccc(-c2cc(=O)c3ccc(OCc4ccccc4)c(OCc4ccccc4)c3o2)cc1 ; O=C(ON1C(=O)CCC1=O)c1ccc(-c2cc(=O)c3ccc(O)c(O)c3o2)cc1]

Benzyl ether 12 (400 mg, 0.549 mmol) was dissolved MeOH/THF (1:3)(60 mL) and the reaction mixture was degassed by purging with nitrogen, then 30% Pd/C (120 mg, 10% w/w) was added the reaction mixture was purged with hydrogen gas from a balloon and stirred under slight positive pressure of hydrogen (balloon) at room temperature for 2 h. LCMS showed complete deprotection. Reaction mixture was filtered through celite, washed with THF and concentrated. Resulting solid was triturated with MeOH, filtered, and dried under high vacuum to obtain diol 13 (150 mg 55%) as a beige solid. NMR and LCMS (m/z=396 M+1) are corresponding with product.

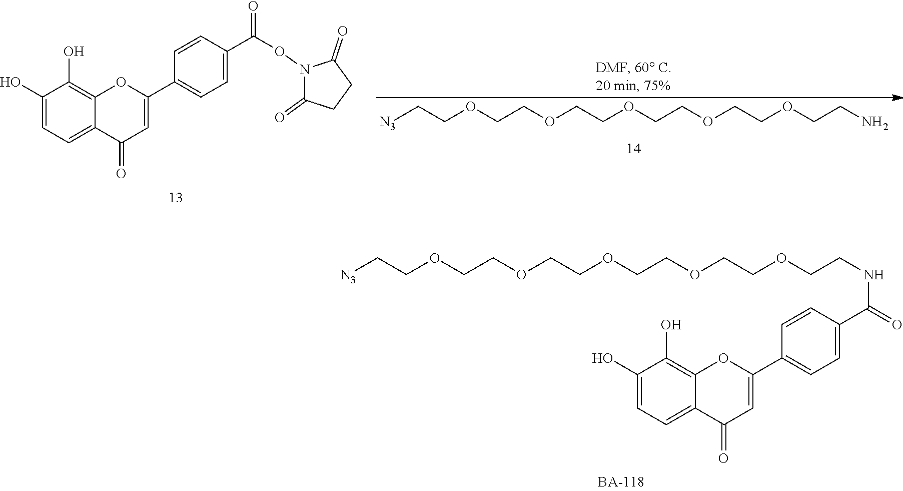 [SMILES: O=C(ON1C(=O)CCC1=O)c1ccc(-c2cc(=O)c3ccc(O)c(O)c3o2)cc1 ; [N-]=[N+]=NCCOCCOCCOCCOCCOCCN ; [N-]=[N+]=NCCOCCOCCOCCOCCOCCNC(=O)c1ccc(-c2cc(=O)c3ccc(O)c(O)c3o2)cc1]

A solution of NHS ester 13 (144 mg, 0.365 mmol, 1 eq), and azido amine 223 mg (2 eq.) in DMF (2 mL) was stirred for 20 min at 60° C. LCMS showed amide formation. Reaction mixture was concentrated, and the crude was purified by flash chromatography using Ethyl acetate/MeOH, 0-5% as an eluent, pure fractions were combined and concentrated to obtain azide BA-118 (160 mg, 75%) as a brown solid. NMR and LCMS m/z 587 (M+1) are corresponding with the product. ¹H NMR (499 MHZ, DMSO-d₆) δ 8.71 (t, J=5.6 Hz, 1H), 8.25 (d, J=8.6 Hz, 2H), 8.02 (d, J=8.6 Hz, 2H), 7.41 (d, J=8.6 Hz, 1H), 6.99 (s, 1H), 6.96 (d, J=8.6 Hz, 1H), 3.60-3.48 (m, 20H), 3.45 (q, J=5.8 Hz, 2H), 3.40-3.34 (m, 2H).

BA-118 was conjugated to an oligo sense strand according to general procedure Type II. MW calculated: 7886.30. MW Found: 7885.05.

### Example 4: BA-118 Conjugate

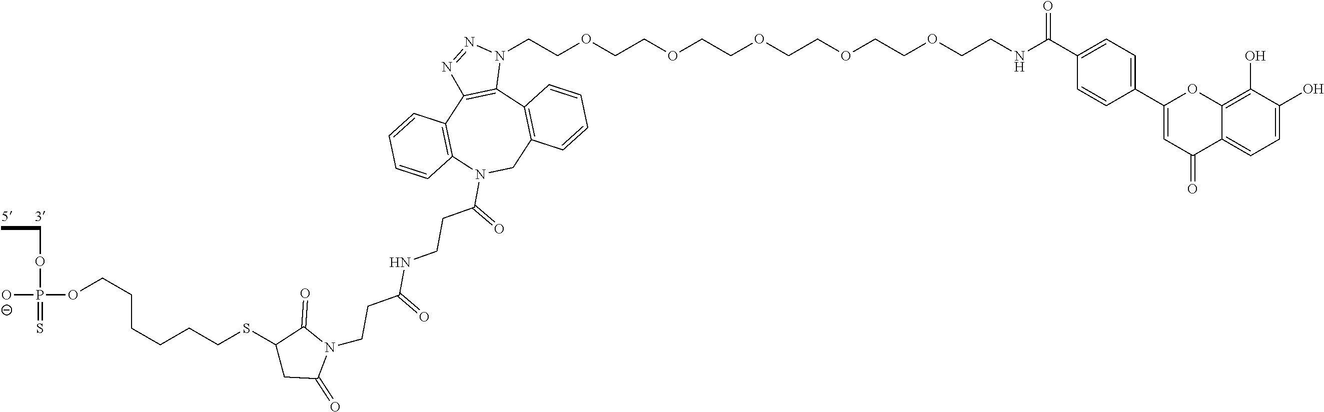

BA-118 azide was prepared according to the experimental procedure described in Example 3. BA-118 was conjugated to an oligo sense strand according to general procedure Type I. MW calculated: 8453.83. MW Found: 8452.74.

### Example 5: BA-129 Conjugate

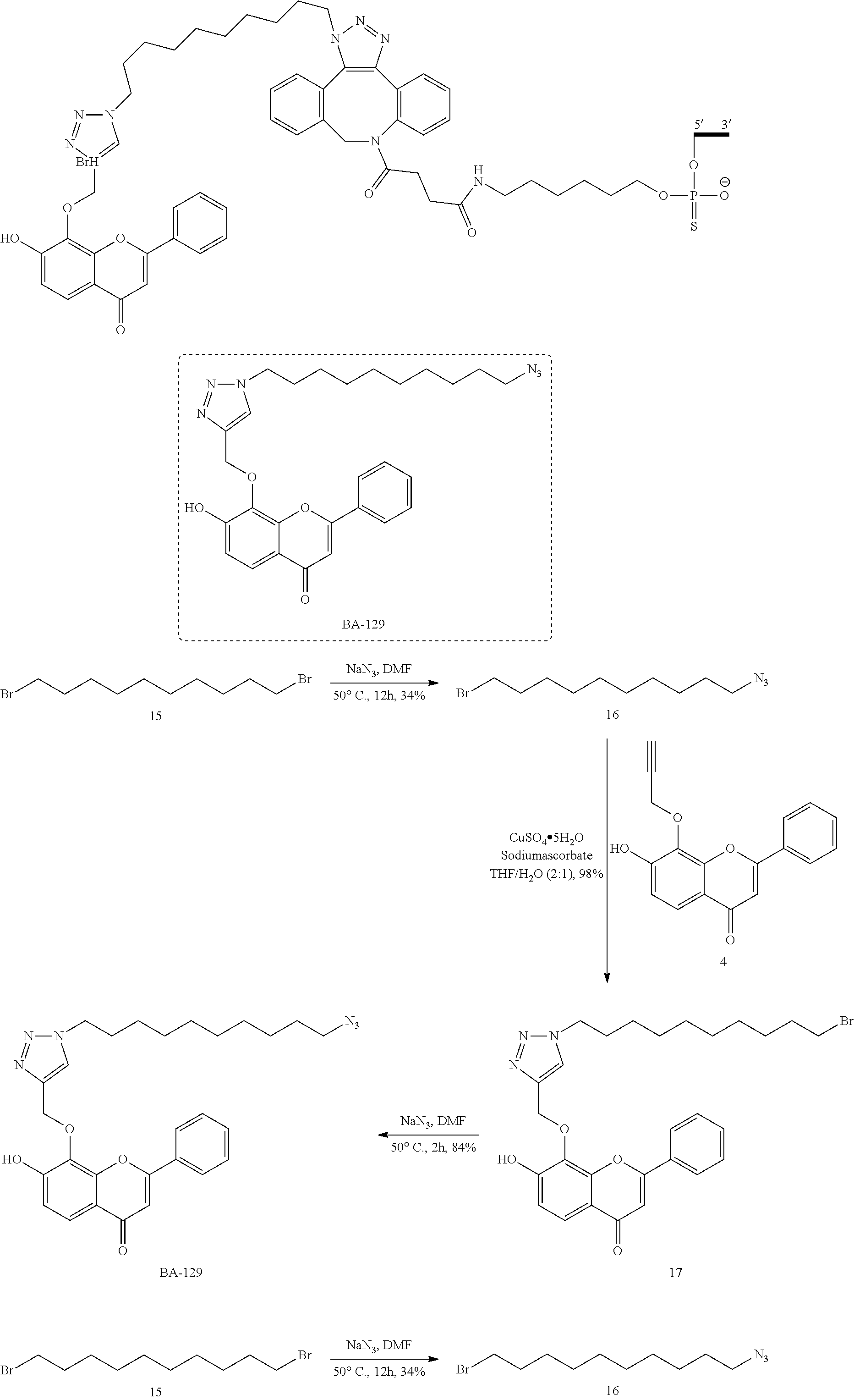

Di-bromodecane 15 (5 g, 16.66 mmol, 1 eq), and Sodium azide (0.542 g, 8.333 mmol, 1 eq) in dry DMF (30 mL) was stirred for 12 h at room temperature, Reaction mixture was cooled and diluted with water and 200 mL, extracted with ethyl acetate 200 mL, dried over Na₂SO₄ and evaporated, resulting crude was purified by column chromatography using 0-10% EtOAc/Hexane, pure fractions were combined and concentrated to obtain azide 16 (1.5 g, 34%) as an oil. LCMS and NMR are corresponding with product.

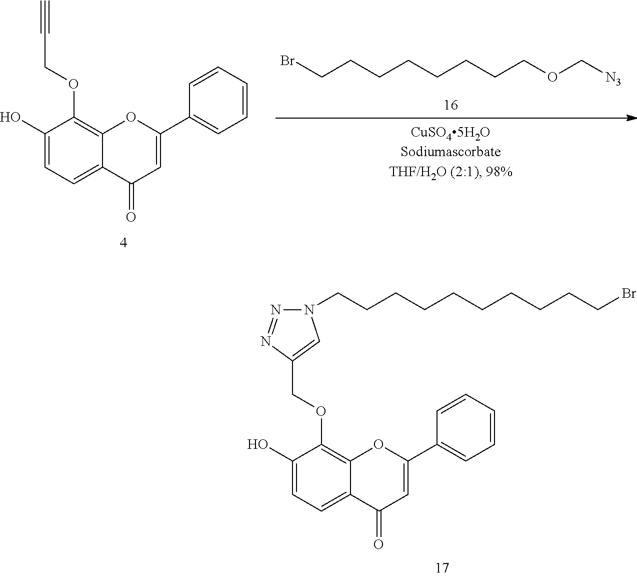 [SMILES: C#CCOc1c(O)ccc2c(=O)cc(-c3ccccc3)oc12 ; [N-]=[N+]=NCOCCCCCCCCBr ; O=c1cc(-c2ccccc2)oc2c(OCc3cn(CCCCCCCCCCBr)nn3)c(O)ccc12]

To a solution of alkyne 4 (0.15 g, 0.514 mmol, 1 eq) and azide 16 (0.162 g, 0.616 mmol, 1.2 eq) in THF (5 mL) at RT was added a solution of CuSO₄·5H₂O (64 mg, 0.257 mmol, 0.5 eq) in water (1 mL) followed by a solution of sodium ascorbate (76 mg, 0.385 mmol, 0.75 eq) in water (1 mL). The reaction was stirred for 2 hrs at RT. Then the reaction was diluted with DCM 50 mL and washed with NaHCO₃ (50 mL) The aqueous phase was extracted with DCM (2×100 mL). The organic layers were combined, dried over Na₂SO₄, filtered, and concentrated. The residue was purified by 0-100% EtOAc/Hexane to give 280 mg of triazole 17 (98%) as a gum. NMR and mass m/z=554 (M+) are corresponding with product.

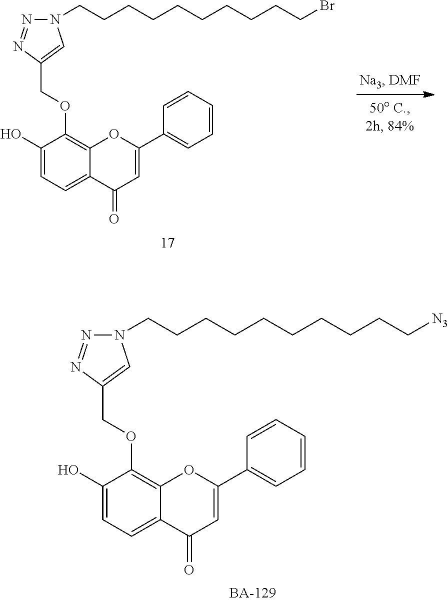 [SMILES: O=c1cc(-c2ccccc2)oc2c(OCc3cn(CCCCCCCCCCBr)nn3)c(O)ccc12 ; [N-]=[N+]=NCCCCCCCCCCn1cc(COc2c(O)ccc3c(=O)cc(-c4ccccc4)oc23)nn1]

A solution of Bromo derivative 17 (0.275 g, 0.496 mmol, 1 eq) and sodium azide (74 mg, 2.482 mmol, 5 eq) in dry DMF (5 mL) under argon was heated to 60° C. for 2 h. LCMS showed complete conversion. The reaction was cooled to room temperature, diluted with aq. Saturated bicarbonate (50 mL) and extracted with EtOAc (100 mL). The combined organic layers were washed with brine (100 mL), dried over Na₂SO₄, and evaporated. The residue was purified by silica gel chromatography, eluting with 0-100% EtOAc/Hexane to afford azide BA-129 as a beige solid (220 mg, 84%) confirmed by NMR and LCMS m/z 517 (M+1). 99% HPLC purity. ¹H NMR (499 MHz, DMSO-d₆) δ 10.80 (s, 1H), 8.10 (s, 1H), 8.01-7.94 (m, 2H), 7.65 (d, J=8.8 Hz, 1H), 7.61-7.52 (m, 3H), 7.04 (d, J=8.7 Hz, 1H), 6.87 (s, 1H), 5.26 (s, 2H), 4.20 (t, J=7.0 Hz, 2H), 3.30 (t, J=7.0 Hz, 2H), 1.59 (p, J=7.1 Hz, 2H), 1.51 (dq, J=8.3, 6.8 Hz, 2H), 1.32-1.07 (m, 10H), 1.04 (q, J=6.9 Hz, 2H).

BA-129 was conjugated to an oligo sense strand according to general procedure Type II. MW calculated: 7816.67. MW Found: 7815.27.

### Example 6: BA-132 Conjugate

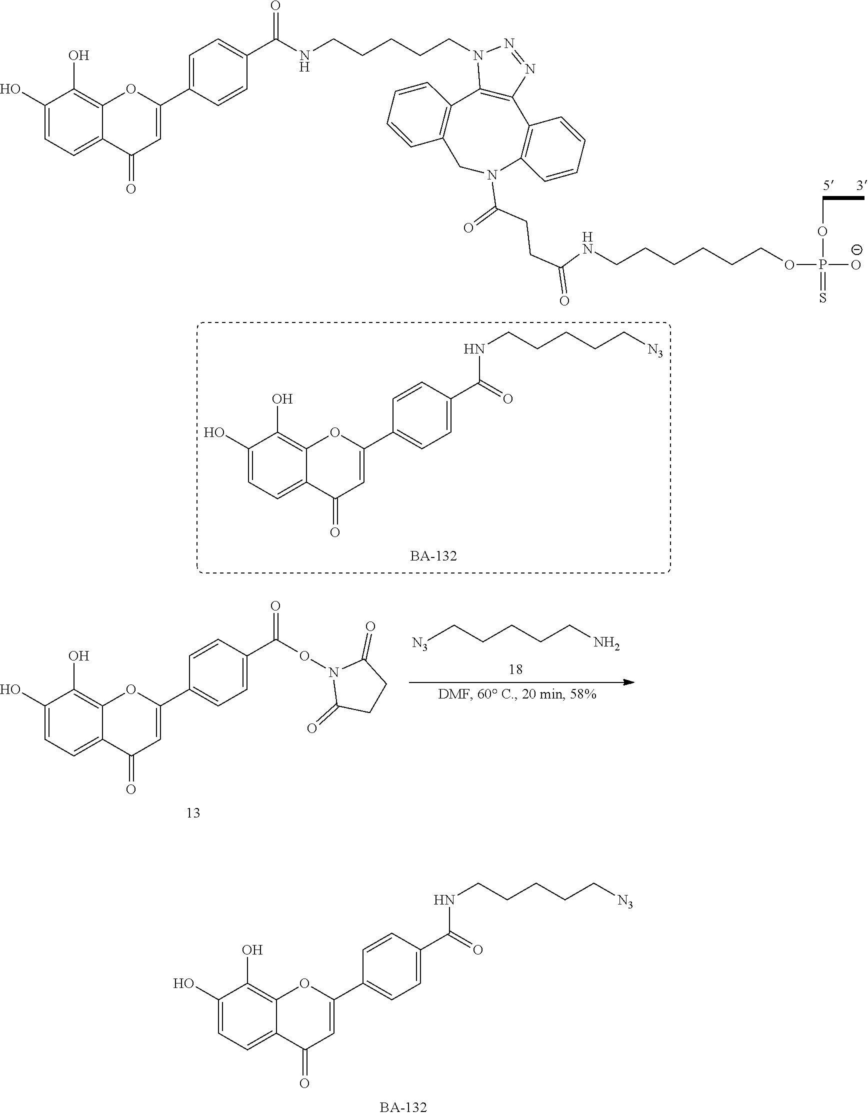

A solution of NHS ester 13 (75 mg, 0.190 mmol, 1 eq), DIPEA (0.079 mL, 0.570 mmol, 3 eq) and azido amine (49 mg, 0.380 mmol, 2 eq) in DMF (1 mL) was stirred for 20 min at 60° C. LCMS showed amide formation. Reaction mixture was concentrated, and the crude was triturated with MeOH, filtered, and dried to obtain amide BA-132 (45 mg, 58% with 95% purity) as a brown solid. NMR and LCMS, Mass m/z 409 (M+1) are corresponding with the product. ¹H NMR (499 MHz, DMSO-d₆) δ 8.64 (t, J=5.7 Hz, 1H), 8.25 (d, J=8.4 Hz, 2H), 8.00 (d, J=8.4 Hz, 2H), 7.41 (d, J=8.6 Hz, 1H), 6.99 (s, 1H), 6.96 (d, J=8.7 Hz, 1H), 3.32 (q, J=7.5 Hz, 4H), 1.58 (pd, J=7.1, 4.7 Hz, 4H), 1.47-1.30 (m, 2H).

BA-132 was conjugated to an oligo sense strand according to general procedure Type II. MW calculated: 7708.48. MW Found: 7706.81.

### Example 7: BA-133 Conjugate

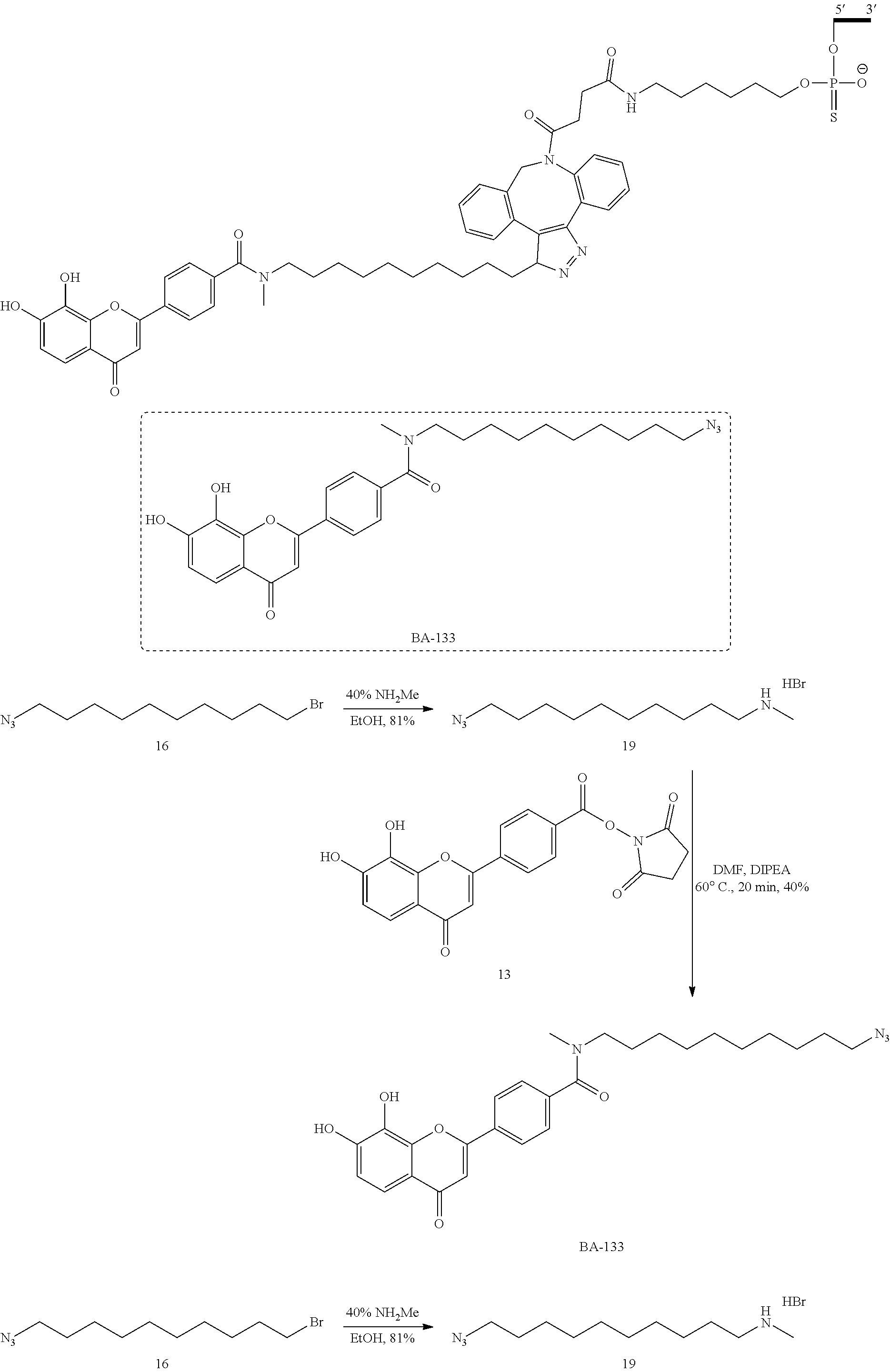

A mixture of Bromide 16 (2 g, 8.547 mmol, 1 eq), and Methyl amine (2.65 g, 34.188 mmol, 4 eq) in Ethanol (20 mL) was stirred for 12 h at 100° C. Reaction mixture was concentrated, and the residue was triturated with ethyl acetate/Hexane, precipitated solids were filtered and dried under high vacuum, to obtain 835 mg 82% amino azide 19 as a white solid. NMR and LCMS m/z 213 M+1, are corresponding with product.

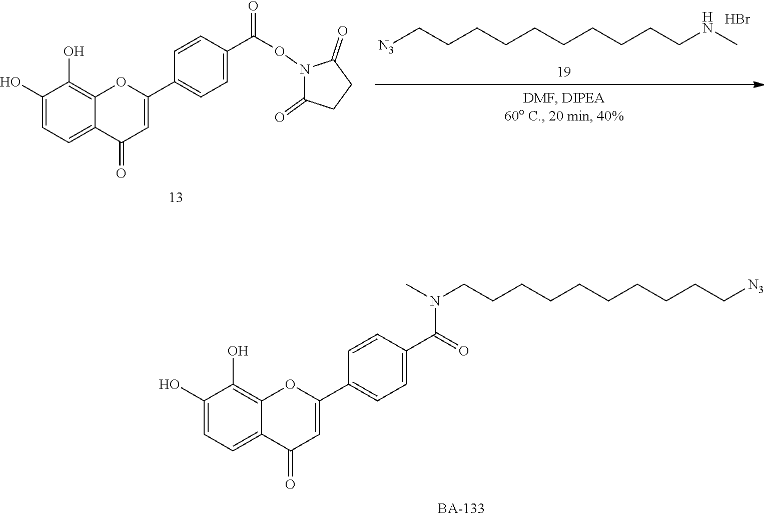 [SMILES: O=C(ON1C(=O)CCC1=O)c1ccc(-c2cc(=O)c3ccc(O)c(O)c3o2)cc1 ; CNCCCCCCCCCCN=[N+]=[N-] ; Br ; CN(CCCCCCCCCCN=[N+]=[N-])C(=O)c1ccc(-c2cc(=O)c3ccc(O)c(O)c3o2)cc1]

A solution of NHS ester 13 (30 mg, 0.076 mmol, 1 eq), DIPEA (0.04 mL, 0.228 mmol, 3 eq) and azido amine 19 (45 mg, 0.152 mmol, 2 eq) in DMF (1 mL) was stirred for 20 min at 60° C. LCMS showed amide formation. Reaction mixture was concentrated, and the crude was purified by column chromatography using 0-20% MeOH-DCM as an eluent, pure fractions were combined and concentrated to obtain amide BA-133 (20 mg, 40% with 90% purity) as a brown solid. NMR and LCMS, Mass m/z 493 (M+1) are corresponding with the product. ¹H NMR (499 MHZ, DMSO-d₆) δ 8.21 (d, J=8.0 Hz, 2H), 7.55 (t, J=9.0 Hz, 2H), 7.40 (d, J=8.6 Hz, 1H), 7.05-6.87 (m, 2H), 3.32 (s, 5H), 3.19 (m, 2H), 1.67-0.93 (m, 16H).

BA-133 was conjugated to an oligo sense strand according to general procedure type II. MW calculated: 7792.65. MW Found: 7791.18.

### Example 8: BA-135 Conjugate

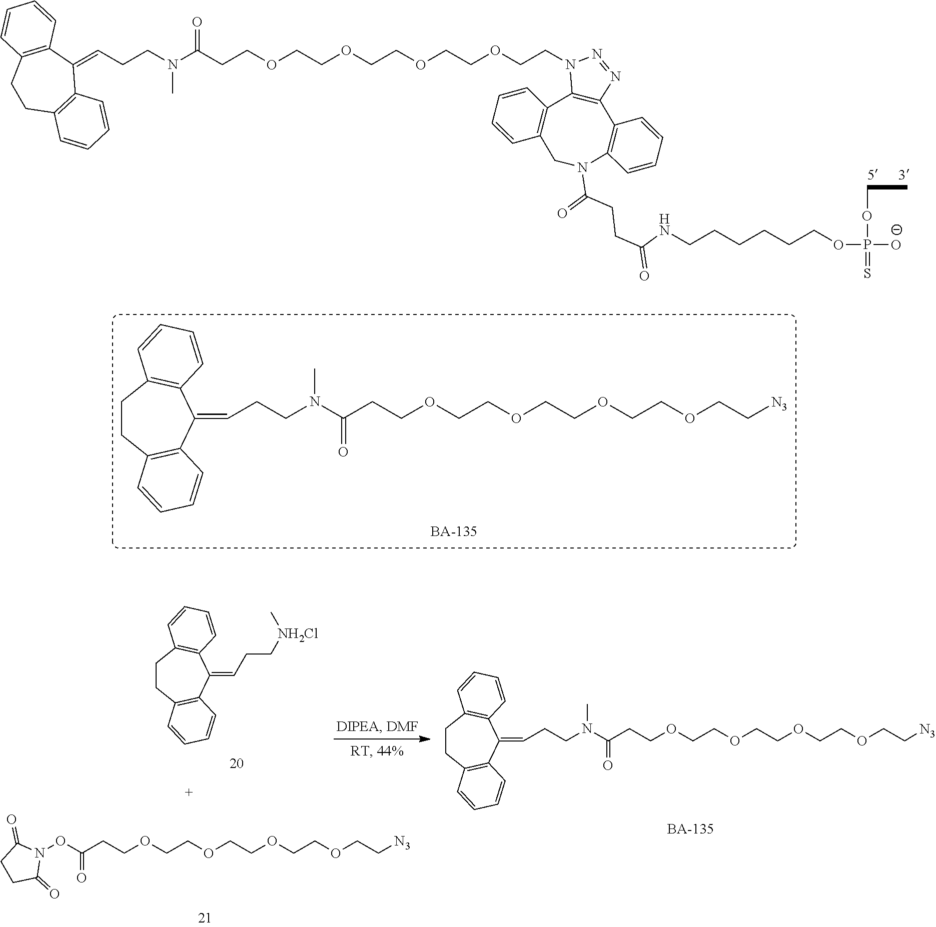

To a stirred solution of Amine-HCl salt 20 (200 mg, 0.669 mmol, 1 eq), and DIPEA (0.23 mL, 1.338 mmol, 2 eq) in DMF (2 mL) was added NHS ester 21 (253 mg, 0.736 mmol, 1.1 eq). Resulting mixture was stirred for 30 min. at RT. LCMS showed amide formation. Reaction mixture was diluted with water 20 mL, extracted with DCM 2×50 mL, combined organic extracts were dried and concentrated, the crude was purified by column chromatography using 0-10% MeOH/DCM as eluent pure fractions were combined and concentrated to obtain amide (160 mg, 45% with 95% purity) as pale-yellow oil. NMR and LCMS, Mass m/z 537 (M+1) are corresponding with the product. ¹H NMR (499 MHz, DMSO-d₆) δ 7.30-6.97 (m, 8H), 5.90-5.71 (m, 1H), 3.58 (ddd, J=6.7, 4.0, 1.6 Hz, 3H), 3.56-3.42 (m, 10H), 3.42-3.35 (m, 4H), 3.32 (m, 2H) 2.84 (s, 2H), 2.65 (s, 2H), 2.40 (d, J=7.7 Hz, 2H), 2.26 (br.d, J=35.2 Hz, 4H).

BA-135 was conjugated to an oligo sense strand according to general procedure Type II. MW calculated: 7836.74. MW Found: 7835.36.

### Example 9: BA-136 Conjugate

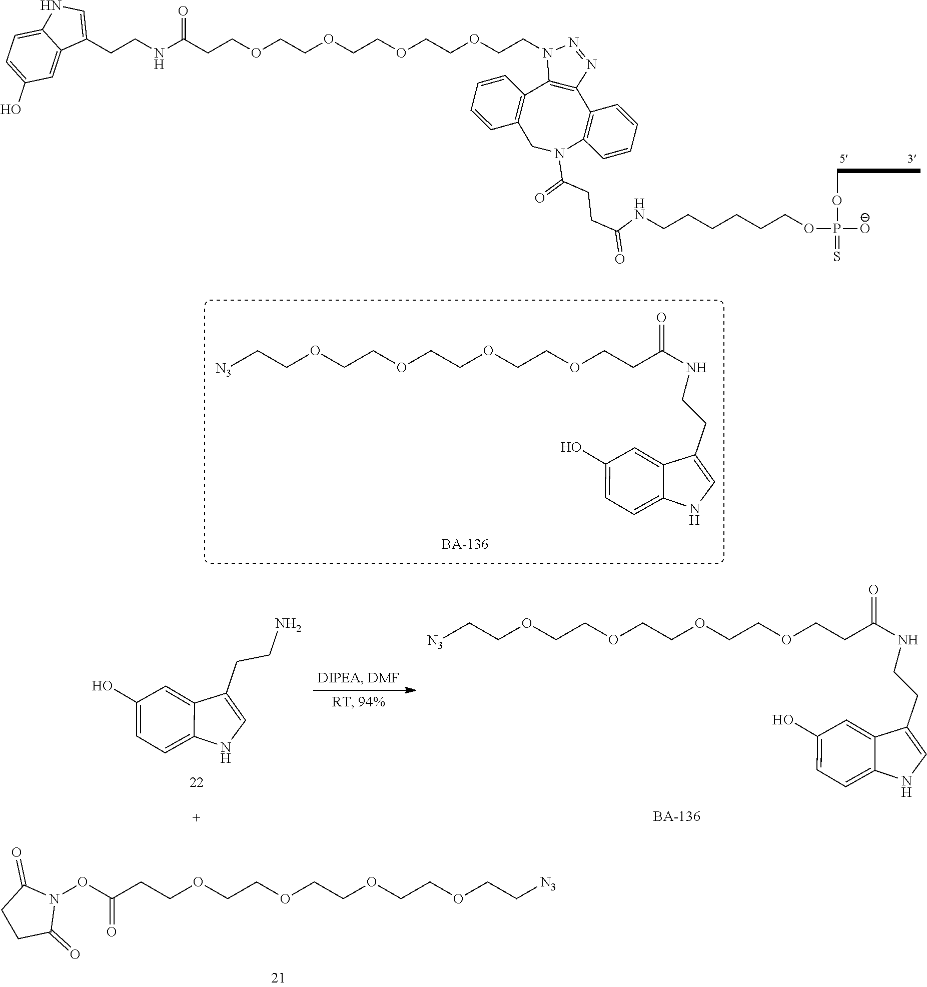

To a stirred solution of Amine 22 (55 mg, 0.313 mmol, 1 eq), and DIPEA (0.1 mL, 0.625 mmol, 2 eq) in DMF (1 mL) was added NHS ester 21 (118 mg, 0.344 mmol, 1.1 eq). Resulting mixture was stirred for 30 min. at RT. LCMS showed amide formation. Reaction mixture was diluted with water 20 mL, extracted with DCM 2×50 mL, combined organic extracts were dried and concentrated, the crude was purified by column chromatography using 0-20% MeOH/DCM as eluent pure fractions were combined and concentrated to obtain amide BA-136 (133 mg, 94% with 95% purity) as a clear oil. NMR and LCMS, Mass m/z 450 (M+1) are corresponding with the product. ¹H NMR (499 MHz, DMSO-d₆) δ 10.46 (d, J=2.5 Hz, 1H), 8.56 (s, 1H), 7.91 (t, J=5.7 Hz, 1H), 7.11 (d, J=8.6 Hz, 1H), 7.02 (d, J=2.4 Hz, 1H), 6.81 (d, J=2.3 Hz, 1H), 6.58 (dd, J=8.6, 2.3 Hz, 1H), 3.63-3.56 (m, 4H), 3.56-3.44 (m, 12H), 3.37 (dd, J=5.6, 4.3 Hz, 2H), 3.30-3.28 (m, 2H), 2.71 (t, J=7.6 Hz, 2H), 2.31 (t, J=7.6 Hz, 2H).

BA-136 was conjugated to an oligo sense strand according to general procedure Type II. MW calculated: 7749.58. MW Found: 7748.02.

### Example 10: BA-137 Conjugate

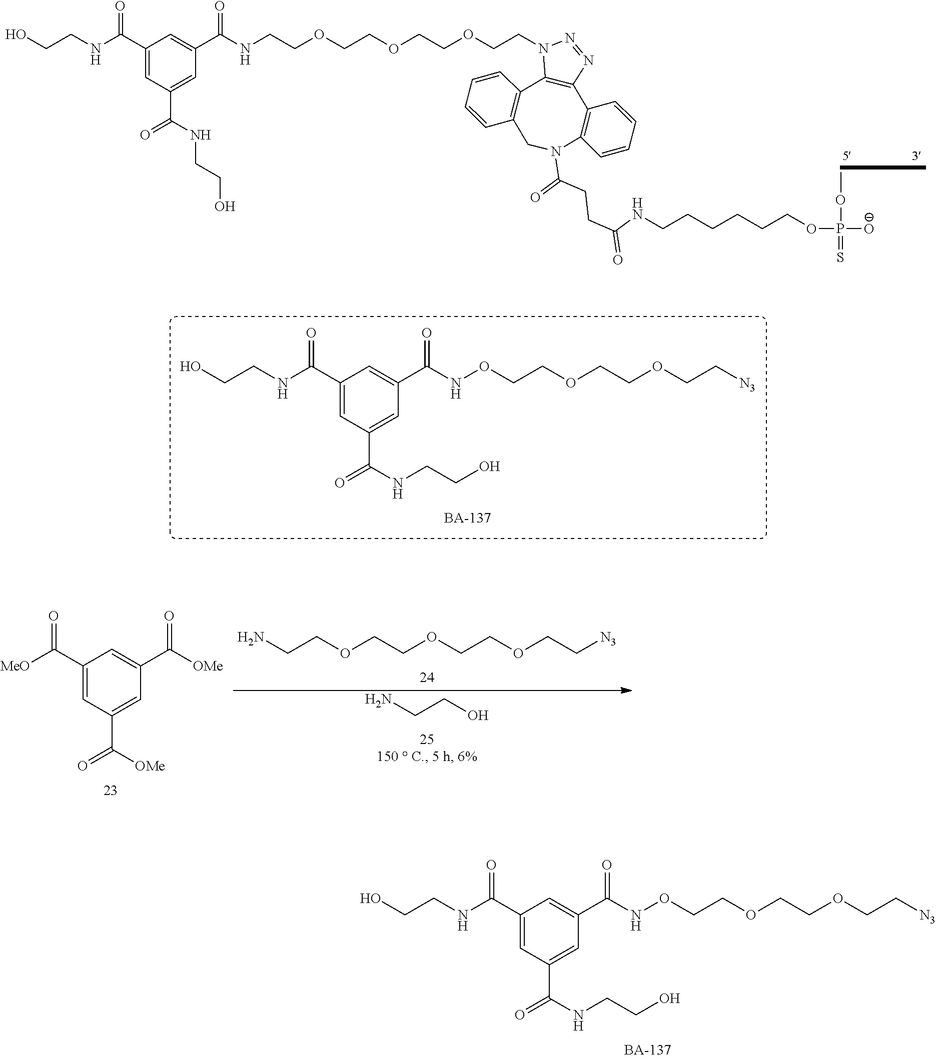

A mixture of tri-ester 23 (0.5 g, 2.294 mmol, 1 eq), and PEG-3-Azidoamine 24 (578 mg, 2.294 mmol, 1 eq). was stirred for 2 h at 150° C. LCMS showed mono amide as major along with about 20% diamide. Reaction mixture was cooled and was added Aminoethanol 25 (1.4 mL, 22.936 mmol, 10 eq) and the mixture was stirred at 150° C. for 3 h. LCMS showed tris-amide as major product. Reaction mixture was diluted with saturated NaCl 20 mL, extracted with DCM 5×100 mL, combined organic extracts were dried and concentrated, the crude was purified by column chromatography using 0-20% MeOH/DCM as eluent pure fractions were combined and concentrated to obtain tris-amide (70 mg, 6% with 95% purity) as a pale-yellow oil. NMR and LCMS, Mass m/z 450 (M+1) are corresponding with the product. ¹H NMR (499 MHz, DMSO-d₆) δ 8.70 (t, J=5.6 Hz, 1H), 8.61 (t, J=5.6 Hz, 2H), 8.44-8.39 (m, 3H), 4.76 (t, J=5.6 Hz, 2H), 3.60-3.50 (m, 14H), 3.45 (q, J=5.8 Hz, 2H), 3.41-3.33 (m, 6H).

BA-137 was conjugated to an oligo sense strand according to general procedure Type II. MW calculated: 7796.59. MW Found: 7795.09.

### Example 11: BA-144 Conjugate

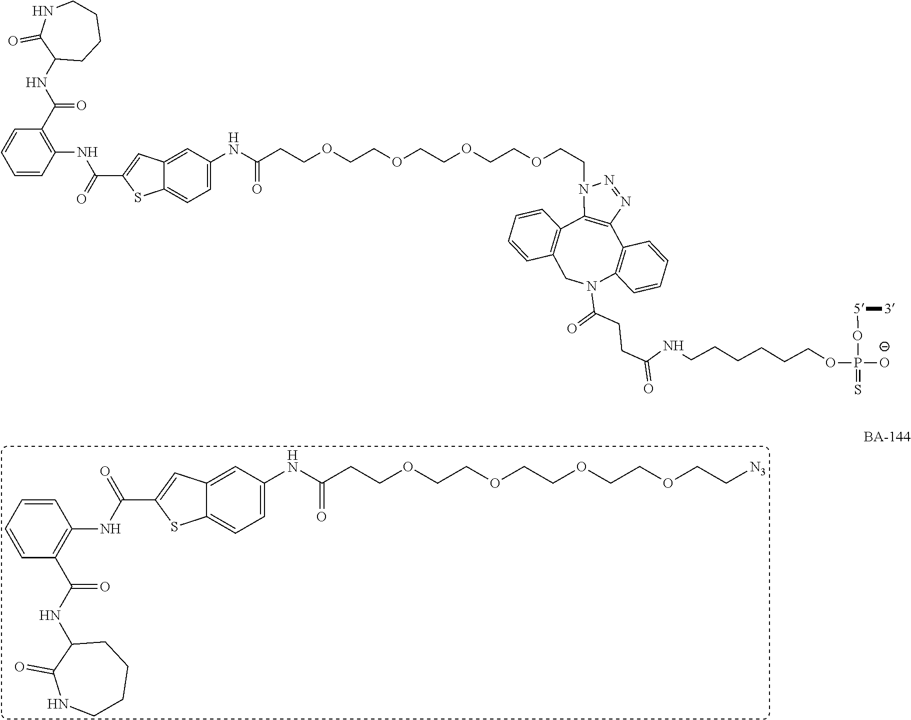

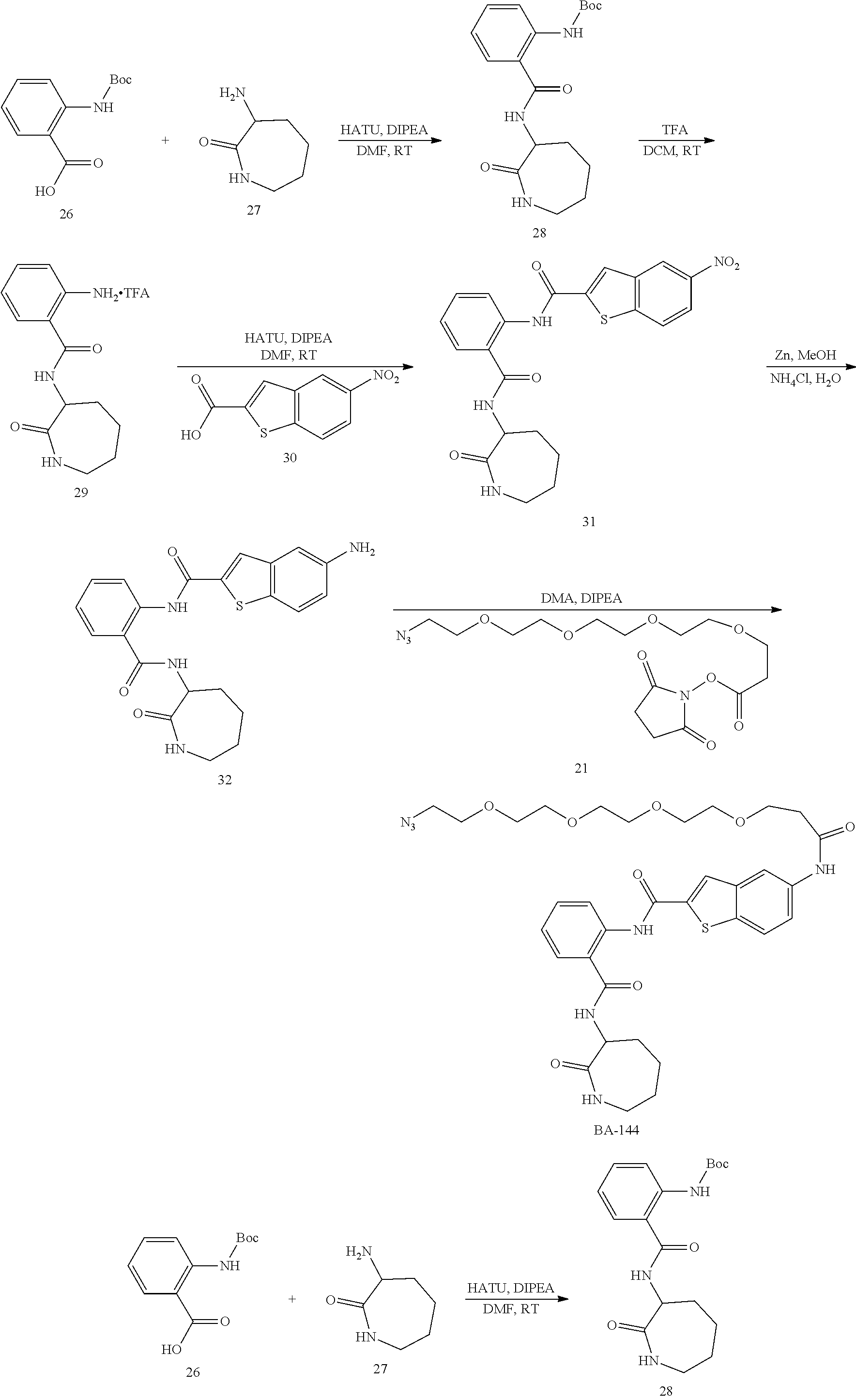

The acid 26 (2.01 g, 8.4 mmol) and amine 27 (1.19 g, 9.27 mmol, 1.1 equiv.) were dissolved in DMF (10 mL) and treated with DIPEA (3.0 mL, 16.9 mmol, 2 equiv.) and HATU (4.8 g, 12.6 mmol, 1.5 equiv.). The reaction mixture was stirred at 20° C. for 3 h. LCMS data showed the desired product. The mixture was poured into water (260 mL). The resulting slurry was sonicated briefly and aged at room temp for 30 min. Filtration, washing the solids with more water (2×50 mL) and air-drying yielded 28 (2.71 g, 92%) as a greyish-white solid. LCMS data: t=2.50 min, MS(+) m/z: 348.3 for [C₁₈H₂₆N₃O₄]⁺ [M+H]⁺; 370.4 for [C₁₈H₂₅NaN₃O₄]⁺ [M+Na]⁺; 717.2 for [2M+Na]⁺; 248.1 for [M+H−Boc]⁺. ¹H NMR (499 MHz, DMSO-d₆) δ 10.52 (s, 1H), 8.51 (d, J=7.0 Hz, 1H), 8.18 (dd, J=8.4, 1.2 Hz, 1H), 7.86 (dd, J=7.4, 4.9 Hz, 1H), 7.76 (dd, J=7.9, 1.5 Hz, 1H), 7.47 (ddd, J=8.6, 7.3, 1.6 Hz, 1H), 7.08 (td, J=7.6, 1.2 Hz, 1H), 4.60 (ddd, J=11.3, 7.1, 1.7 Hz, 1H), 3.25 (ddd, J=15.7, 11.2, 4.9 Hz, 1H), 3.10 (dt, J=13.8, 6.3 Hz, 1H), 1.97-1.83 (m, 2H), 1.79 (dd, J=14.2, 4.3 Hz, 1H), 1.71 (qt, J=12.7, 3.5 Hz, 1H), 1.58 (qd, J=13.1, 2.6 Hz, 1H), 1.46 (s, 9H), 1.32-1.18 (m, 1H).

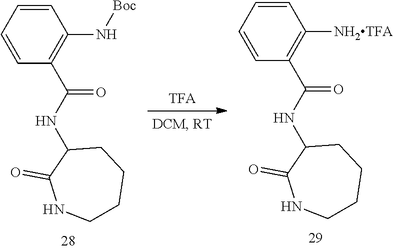

The carbamate 28 (2.71 g, 7.8 mmol) was dissolved in DCM (20 mL) and treated with TFA (6.0 mL, 78 mmol, 10 equiv.). The reaction mixture was stirred at 20° C. for 18 h. LCMS data showed the desired product. The mixture was concentrated to dryness, redissolved in 1:1 DCM/EtOAc and reconcentrated to yield amine 29 (2.74 g, 102%) as a thick brown oil. LCMS data: t=1.26 min, MS(+) m/z: 248.9 for [C₁₃H₁₈N₃O₂]⁺ [M+H]⁺. ¹H NMR (499 MHZ, DMSO-d₆) δ 8.31 (d, J=6.9 Hz, 1H), 7.91 (dd, J=7.4, 4.9 Hz, 1H), 7.65 (dd, J=7.8, 1.5 Hz, 1H), 7.36 (ddd, J=8.5, 7.2, 1.5 Hz, 1H), 7.03 (dd, J=8.2, 1.2 Hz, 1H), 6.97 (td, J=7.5, 1.2 Hz, 1H), 4.60 (dt, J=10.4, 3.9 Hz, 1H), 3.23 (ddd, J=15.7, 11.2, 4.9 Hz, 1H), 3.16-3.05 (m, 1H), 1.91 (tt, J=16.0, 3.0 Hz, 2H), 1.78 (dt, J=13.5, 4.2 Hz, 1H), 1.69 (qt, J=15.4, 4.2 Hz, 1H), 1.62-1.51 (m, 1H), 1.32-1.18 (m, 1H).

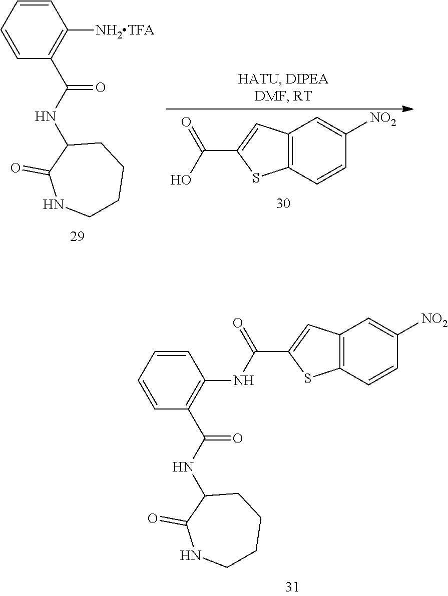

The acid 30 (0.5 g, 2.1 mmol) and amine 29 (1 g, 3.2 mmol, 1.5 equiv.) were dissolved in DMF (10 mL) and treated with DIPEA (1.6 mL, 9 mmol, 4equiv.) and HATU (1.3 g, 3.4 mmol, 1.5 equiv.). The reaction mixture was stirred at 20° C. for 18 h. An off-white ppt has formed. The mixture was poured into 200 mL water and sonicated with swirling for 5-10 min. The resulting precipitate was isolated by filtration. Washing with water and air-drying yielded the desired product amide 31 (340 mg, 33.5%) as a gray powder. LCMS data: t=2.64 min, MS(+) m/z: 453.3 for [C₂₂H₂₁N₄O₅S]⁺ [M+H]⁺; 475.2 for [C₂₂H₂₀N₄NaO₅S]⁺ [M+Na]⁺. MS(−) m/z: 451.2 for [C₂₂H₁₉N₄O₅S]− [M−H]. ¹H NMR (499 MHZ, DMSO-d₆) δ 12.41 (s, 1H), 9.07 (d, J=2.3 Hz, 1H), 8.69 (d, J=7.1 Hz, 1H), 8.44 (dd, J=8.3, 1.2 Hz, 1H), 8.41-8.34 (m, 2H), 8.30 (dd, J=8.9, 2.3 Hz, 1H), 7.98-7.88 (m, 2H), 7.60 (ddd, J=8.5, 7.3, 1.5 Hz, 1H), 7.28 (td, J=7.6, 1.2 Hz, 1H), 4.69 (ddd, J=11.3, 7.1, 1.7 Hz, 1H), 3.30-3.21 (m, 1H), 3.17-3.08 (m, 1H), 1.98-1.88 (m, 2H), 1.84-1.75 (m, 1H), 1.80-1.67 (m, 1H), 1.71-1.54 (m, 1H), 1.33-1.20 (m, 1H).

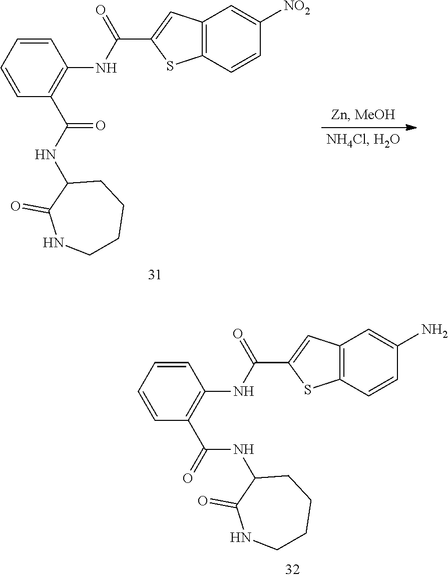 [SMILES: O=C(Nc1ccccc1C(=O)NC1CCCCNC1=O)c1cc2cc([N+](=O)[O-])ccc2s1 ; Nc1ccc2sc(C(=O)Nc3ccccc3C(=O)NC3CCCCNC3=O)cc2c1]

The nitroarene 31 (0.2 g, 0.44 mmol) and zinc metal (0.057 g, 0.9 mmol, 2 equiv.) were suspended in methanol (9 mL) and water (1 mL) and treated with ammonium chloride (0.059 g, 1.1 mmol, 2.5 equiv.). The reaction mixture was stirred at 20° C. overnight. LCMS data is consistent with the presence of the desired product. The mixture was treated with sat. NaHCO₃ (5 mL). The reaction mixture was sonicated and swirled until relatively homogenous (zinc metal is still present on the bottom of the reaction vessel). Filtration, washing with water and air-drying yielded amine 32 (178 mg, 95%) as a beige solid. LCMS data: t=1.92 min, MS(+) m/z: 423.4 for [C₂₂H₂₃N₄O₃S]⁺ [M+H]⁺; 445.2 for [C₂₂H₂₂NaN₄O₃S]⁺ [M+Na]⁺; 867.5 for [C₄₄H₄₅N₈O₆S₂]⁺ [2M+H]⁺; MS(−) m/z: 421.1 for [C22H21N4O3S]− [M−H]−. An additional minor peak was observed at t=2.13 min, MS(+) m/z: 439.3 [C22H23N4O4S]⁺ [M+H]⁺; MS(−) m/z: 437.3 for [C22H21N4O4S]− [M−H]−, corresponding to the partially reduced hydroxylamine byproduct. The NMR data is consistent with the structure of the desired product: ¹H NMR (499 MHz, DMSO-d₆) δ 12.25 (s, 1H), 8.65 (d, J=6.8 Hz, 1H), 8.46 (d, J=8.4 Hz, 1H), 7.90 (d, J=7.8 Hz, 2H), 7.78 (s, 1H), 7.67 (d, J=8.7 Hz, 1H), 7.58 (s, 1H), 7.24 (t, J=7.4 Hz, 1H), 7.07 (s, 1H), 6.87 (d, J=8.5 Hz, 1H), 5.25 (s, 2H), 4.94 (s, 1H), 4.80 (s, 1H), 4.71 (d, J=7.7 Hz, 1H), 3.14-3.06 (m, 1H), 1.92 (s, 2H), 1.78 (s, 2H), 1.60 (d, J=12.9 Hz, 1H), 1.34-1.16 (m, 1H).

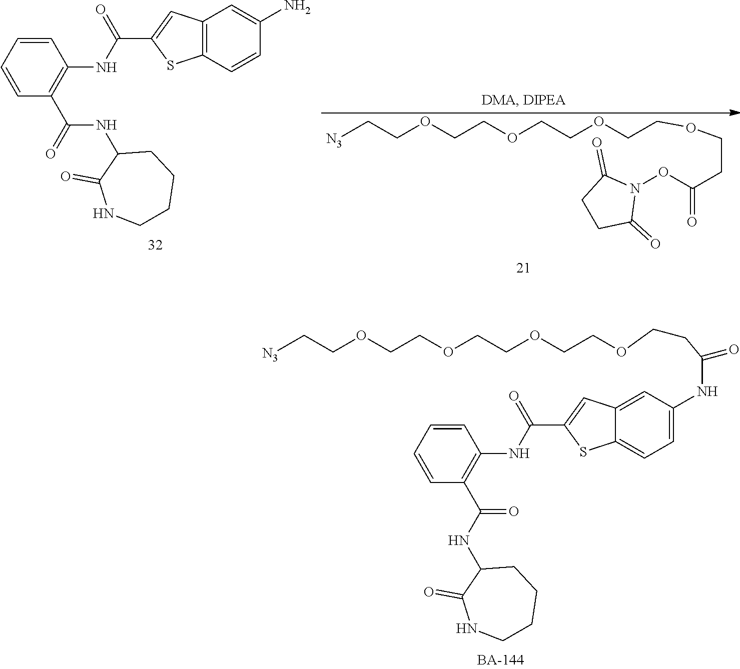 [SMILES: Nc1ccc2sc(C(=O)Nc3ccccc3C(=O)NC3CCCCNC3=O)cc2c1 ; [N-]=[N+]=NCCOCCOCCOCCOCCC(=O)ON1C(=O)CCC1=O ; [N-]=[N+]=NCCOCCOCCOCCOCCC(=O)Nc1ccc2sc(C(=O)Nc3ccccc3C(=O)NC3CCCCNC3=O)cc2c1]

The aniline 32 (0.16 g, 0.4 mmol, 1.5 equiv.) was dissolved/suspended in dry DMA (2 mL) and treated with DIPEA (0.13 mL, 0.77 mmol, 3 equiv.) and NHS—CO(CH₂)₂(PEG)₄N₃ 21 (0.1 g, 0.26 mmol). The reaction mixture was stirred at 20° C. over the weekend. LCMS data showed only partial conversion. HATU (0.5 g) was added and the mixture was stirred overnight. The mixture was diluted with DCM (60 mL) and washed with water (50 mL), sat. NaHCO₃ and sat. NaCl. Drying over Na₂SO₄ and conc. in vacuo yielded a beige solid. LCMS data: t=2.41 min, MS(+) m/z: 696.6 for [C₃₃H₄₂N₇O₈S]⁺ [M+H]⁺, 718.6 for [C₃₃H₄₁N₇NaO₅S]⁺ [M+Na]⁺; MS(−) m/z: 694.6 for [C₃₃H₄₀N₇O₈S]− [M−H]−. Silicagel purification (0% to 25% methanol in ethyl acetate) yielded the desired product (eluted with 11% solvent B, tubes 19-27): fraction 1 (112 mg). Tubes 12-18 and 28-40 also contained the desired product but were not pure enough and were collected separately: fraction 2 (82 mg). ¹H NMR data of fraction 1 showed the material to be contaminated with DMA. The material was dissolved in DCM and concentrated in vacuo. It was kept on the high vacuum manifold overnight to obtain Azide BA-144 (68 mg, 15%) NMR data was consistent with the structure of the desired product. ¹H NMR (499 MHz, DMSO-d₆) δ 12.37 (s, 1H), 10.16 (s, 1H), 8.66 (d, J=7.1 Hz, 1H), 8.50-8.38 (m, 2H), 8.03-7.95 (m, 2H), 7.93-7.88 (m, 2H), 7.59 (ddd, J=10.9, 7.3, 1.8 Hz, 2H), 7.26 (td, J=7.6, 1.2 Hz, 1H), 4.76-4.66 (m, 1H), 3.74 (t, J=6.2 Hz, 2H), 3.57-3.48 (m, 12H), 3.37 (d, J=4.9 Hz, 1H), 3.11 (dd, J=14.4, 7.5 Hz, 1H), 2.61 (t, J=6.4 Hz, 2H), 1.97-1.88 (m, 2H), 1.83-1.68 (m, 1H), 1.60 (q, J=11.9, 11.5 Hz, 1H), 1.27 (t, J=13.1 Hz, 1H), 1.19-1.01 (m, 1H).

BA-144 was conjugated to an oligo sense strand according to general procedure Type II. MW calculated: 7995.86. MW Found: 7994.41.

### Example 12: BA-167 Conjugate

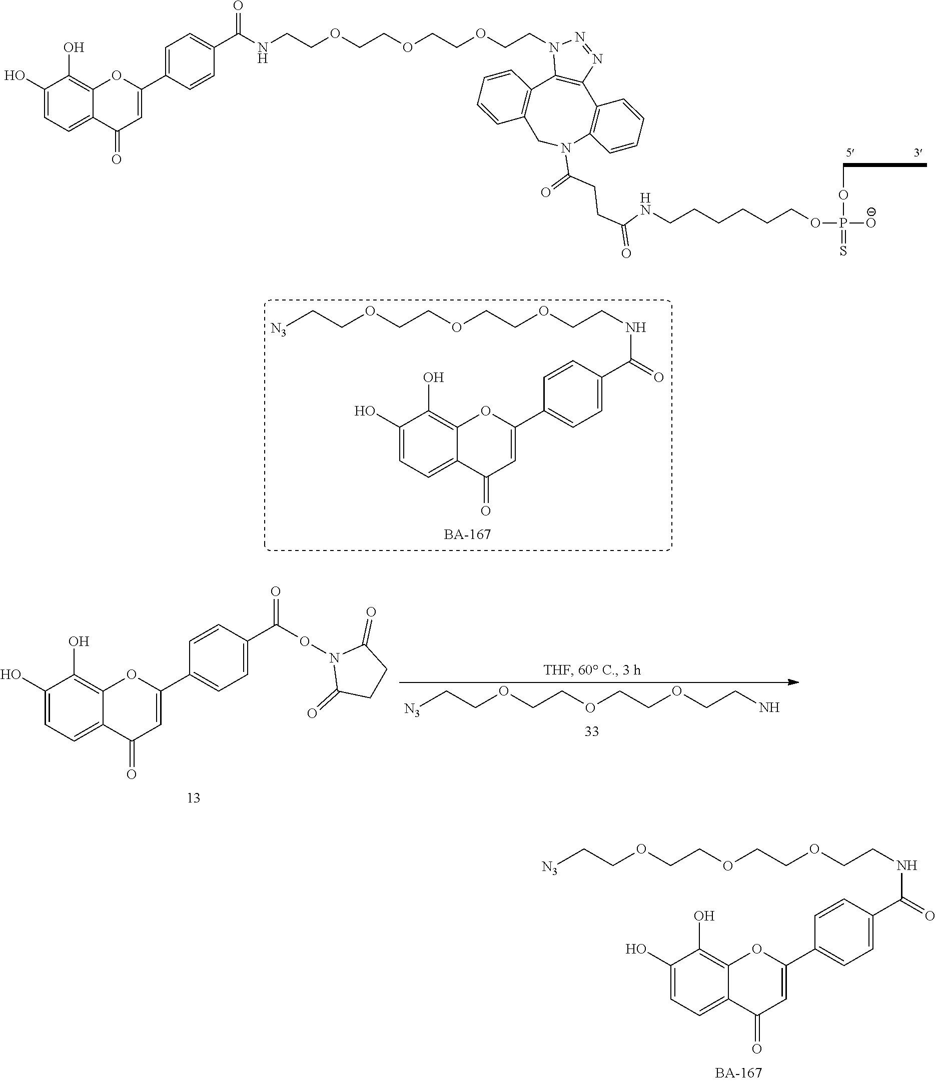

A suspension of NHS ester 13 (665 mg, 1.684 mmol, 1 eq) and azido amine 37 (440 mg, 2 mmol, 1.2 eq) and DIPEA (0.5 ml, 3.7 mmol, 2.2 eq) in THF (20 mL) was stirred for 3 h at 60° C. LCMS showed amide formation. Reaction mixture was concentrated and resulting solids were triturated with MeOH, filtered to obtain (595 mg, 71%) as a beige solid. NMR and LCMS m/z 521 (M+Na) are corresponding with the product. >95% purity by HPLC, LCMS and NMR. ¹H NMR (499 MHz, DMSO-d₆) δ 8.71 (t, J=5.6 Hz, 1H), 8.25 (d, 8.6 Hz, 2H), 8.02 (d, 8.6 Hz, 2H), 7.41 (d, J=8.6 Hz, 1H), 6.99 (s, 1H), 6.96 (d, J=8.7 Hz, 1H), 3.61-3.51 (m, 12H), 3.45 (q, J=5.8 Hz, 2H), 3.39-3.35 (m, 2H).

BA-167 was conjugated to an oligo sense strand according to general procedure Type II. MW calculated: 8209.59. MW Found: 8208.68.

### Example 13: BA-169 Conjugate

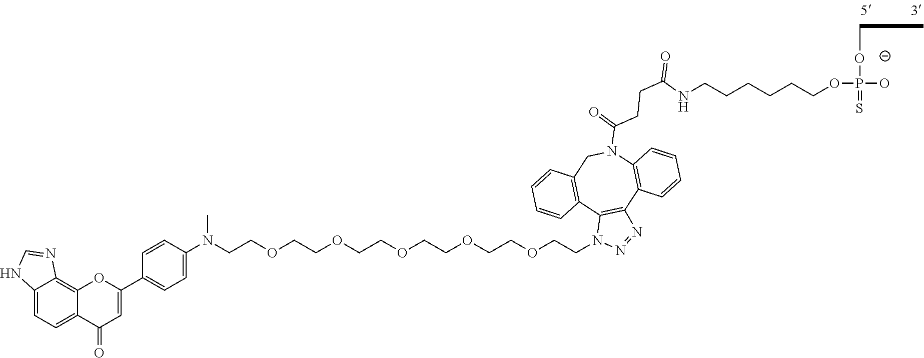

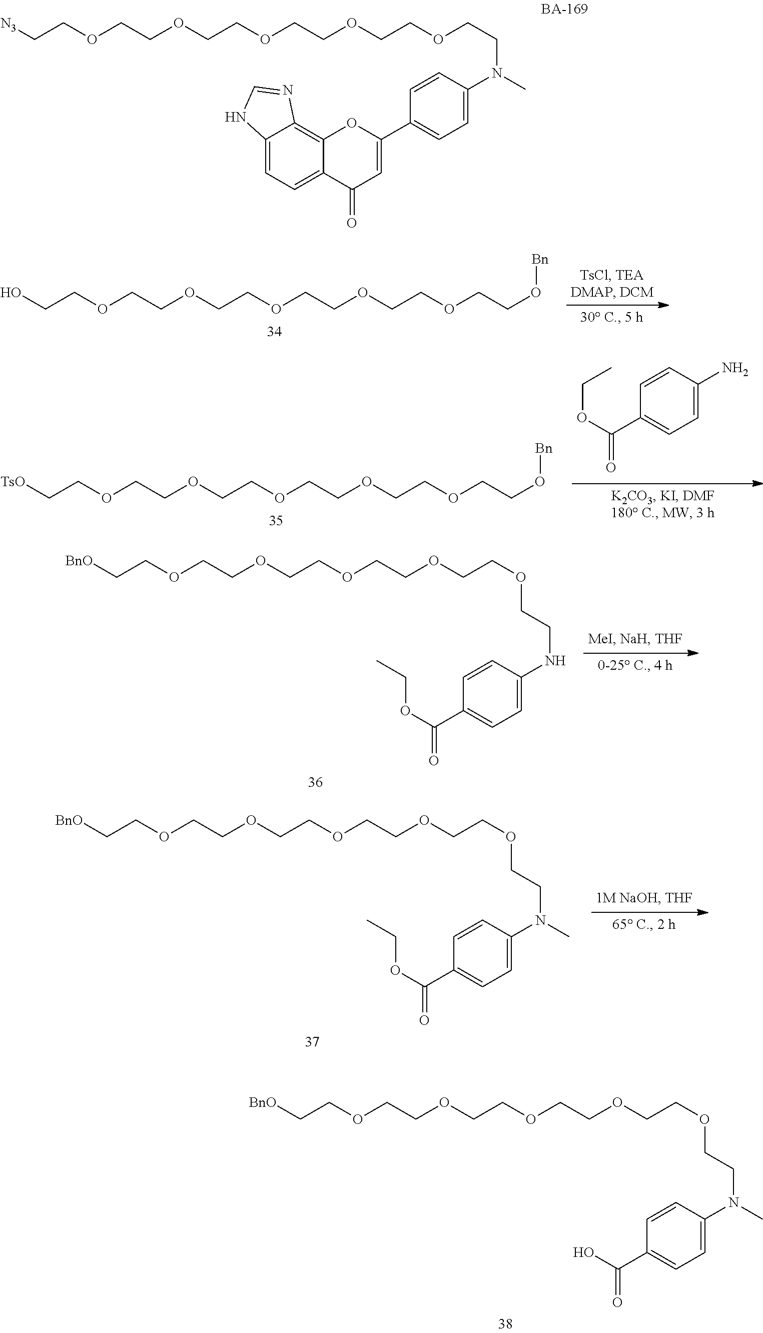

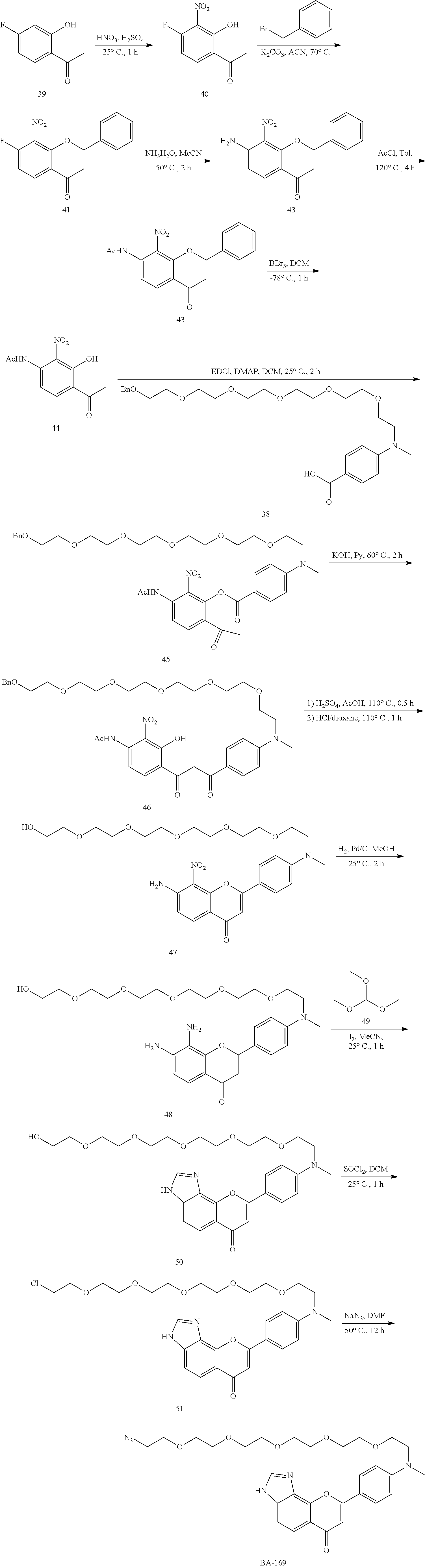

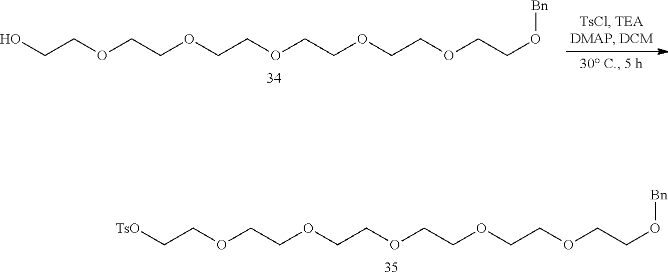 [SMILES: OCCOCCOCCOCCOCCOCCOCc1ccccc1 ; Cc1ccc(S(=O)(=O)OCCOCCOCCOCCOCCOCCOCc2ccccc2)cc1]

To a solution of 2-[2-[2-[2-[2-(2-benzyloxyethoxy)ethoxy]ethoxy]ethoxy]ethoxy]ethanol 34 (5 g, 13.42 mmol, 1 eq) in DCM (100 mL) was added TEA (2.72 g, 26.85 mmol, 3.74 mL, 2 eq), 4-toluenesulfonyl chloride (2.82 g, 14.77 mmol, 1.1 eq) and DMAP (164.01 mg, 1.34 mmol, 0.1 eq). The mixture was stirred at 30° C. for 5 hr. LC-MS showed the starting material was consumed completely and one main peak with desired m/z was detected. The reaction mixture was concentrated under reduced pressure and the residue was purified by flash silica gel chromatography (ISCO®; 40 g SepaFlash® Silica Flash Column, Eluent of 0˜50% Ethyl acetate/Petroleum ether gradient @ 100 mL/min) to give the title compound 35 (6.5 g, 12.34 mmol, 91.94% yield) as a colorless oil. MS ES⁺: 527.1. ¹H NMR (400 MHZ, CDCl₃) δ=7.80 (d, J=8.3 Hz, 2H), 7.38-7.32 (m, 6H), 7.31-7.28 (m, 1H), 4.57 (s, 2H), 4.18-4.14 (m, 2H), 3.69-3.62 (m, 18H), 3.58 (s, 4H), 2.45 (s, 3H).

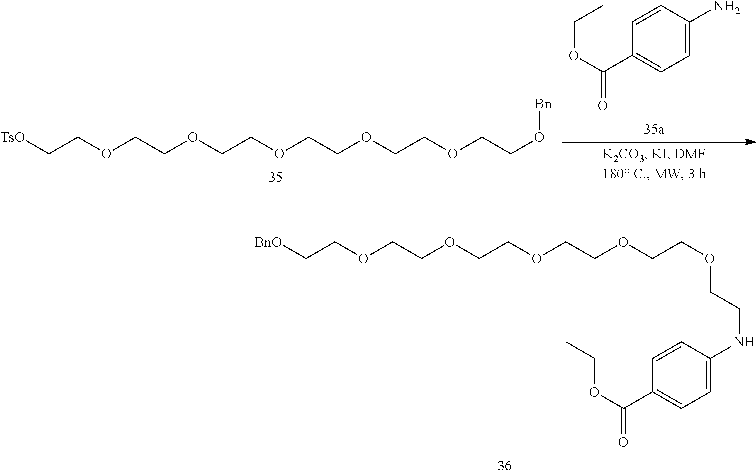 [SMILES: Cc1ccc(S(=O)(=O)OCCOCCOCCOCCOCCOCCOCc2ccccc2)cc1 ; CCOC(=O)c1ccc(N)cc1 ; CCOC(=O)c1ccc(NCCOCCOCCOCCOCCOCCOCc2ccccc2)cc1]

To a solution of 2-[2-[2-[2-[2-(2-benzyloxyethoxy)ethoxy]ethoxy]ethoxy]ethoxy]ethyl 4-methylbenzenesulfonate 35 (3.25 g, 6.17 mmol, 1 eq) and ethyl 4-aminobenzoate 35a (1.02 g, 6.17 mmol, 1.29 mL, 1 eq) in DMF (10 mL) was added K₂CO₃ (2.56 g, 18.51 mmol, 3 eq) and KI (1.02 g, 6.17 mmol, 1 eq). The mixture was stirred at 180° C. for 3 hours under microwave. LC-MS showed the start material was consumed completely and one main peak with desired m/z was detected. The reaction mixture was cooled to room temperature, concentrated and the residue was purified by flash silica gel chromatography (ISCO®; 80 g SepaFlash® Silica Flash Column, Eluent of 0˜60% Ethyl acetate/Petroleum ether gradient @ 100 mL/min) to give the title compound 36 (2 g, 3.85 mmol, 31.18% yield) as a yellow oil. MS ES⁺: 520.2. ¹H NMR (400 MHZ, CDCl₃) δ=7.91-7.82 (m, 2H), 7.34 (d, J=4.4 Hz, 4H), 7.32-7.28 (m, 1H), 6.62 (d, J=8.6 Hz, 2H), 4.56 (s, 2H), 4.32 (q, J=7.1 Hz, 2H), 3.72 (t, J=5.1 Hz, 2H), 3.67-3.64 (m, 18H), 3.64-3.61 (m, 2H), 3.35 (t, J=5.1 Hz, 2H), 1.37 (t, J=7.1 Hz, 3H).

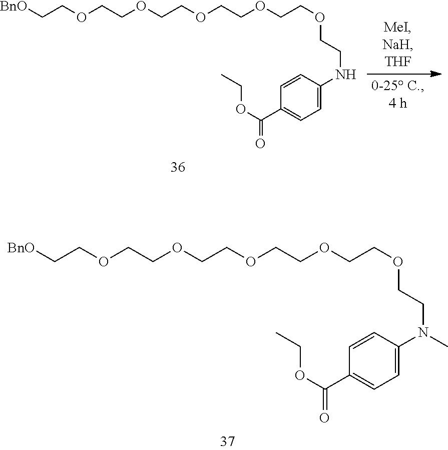 [SMILES: CCOC(=O)c1ccc(NCCOCCOCCOCCOCCOCCOCc2ccccc2)cc1 ; CCOC(=O)c1ccc(N(C)CCOCCOCCOCCOCCOCCOCc2ccccc2)cc1]

To a solution of ethyl 4-[2-[2-[2-[2-[2-(2-benzyloxyethoxy)ethoxy]ethoxy]ethoxy]ethoxy]ethylamino]benzoate 36 (1 g, 1.92 mmol, 1 eq) in THF (10 mL) was added NaH (115.46 mg, 2.89 mmol, 60% purity, 1.5 eq) at 0° C. under N₂, after stirring for 0.5 hr, Mel (1.37 g, 9.62 mmol, 599.02 μL, 5 eq) was added. The mixture was stirred at 25° C. for 4 hr under N₂ (the above reaction was repeated one more time and the two reactions were worked up together). LC-MS showed the starting material was consumed completely and one main peak with desired m/z was detected. The reaction mixture was quenched by addition of aq. NH₄Cl (20 mL) at 0° C., and then diluted with H₂O (50 mL) and extracted with EA (50 mL×3). The combined organic layers were washed with brine (100 mL×1), dried over anhydrous Na₂SO₄, filtered, and concentrated under reduced pressure resulting residue was purified by flash silica gel chromatography (ISCO®; 40 g SepaFlash® Silica Flash Column, Eluent of 0˜60% Ethyl acetate/Petroleum ether gradient @ 100 mL/min) to give the title compound 37 (1.7 g, 3.19 mmol, 82.77% yield) as a yellow oil. MS ES⁺: 534.7. ¹H NMR (400 MHz, CDCl₃) δ=7.94-7.87 (m, 2H), 7.34 (d, J=4.4 Hz, 4H), 7.31-7.28 (m, 1H), 6.70 (d, J=9.0 Hz, 2H), 4.57 (s, 2H), 4.33 (q, J=7.1 Hz, 2H), 3.70-3.59 (m, 24H), 3.07 (s, 3H), 1.37 (t, J=7.1 Hz, 3H).

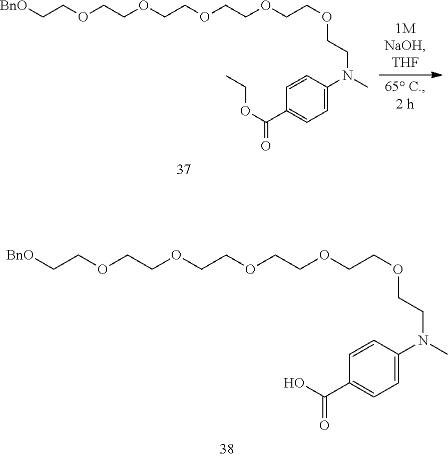 [SMILES: CCOC(=O)c1ccc(N(C)CCOCCOCCOCCOCCOCCOCc2ccccc2)cc1 ; CN(CCOCCOCCOCCOCCOCCOCc1ccccc1)c1ccc(C(=O)O)cc1]

To a solution of ethyl 4-[2-[2-[2-[2-[2-(2-benzyloxyethoxy)ethoxy]ethoxy]ethoxy]ethoxy]ethyl-methyl-amino]benzoate 37 (1.2 g, 2.25 mmol, 1 eq) in THF (12 mL) was added a solution of NaOH (269.82 mg, 6.75 mmol, 3 eq) in H₂O (4 mL). The mixture was stirred at 65° C. for 2 hr. LC-MS showed the starting material was consumed completely and one main peak with desired m/z was detected. The reaction mixture was cooled to room temperature, concentrated under reduced pressure resulting residue was diluted with H₂O (10 mL), acidized by 1N HCl until pH=5-6 and extracted with EA (20 mL×3). The combined organic layers were washed with brine (40 mL×1), dried over anhydrous Na₂SO₄, filtered, and concentrated under reduced pressure to give the title compound 38 (1.1 g, crude) as a yellow oil which was used into the next step without further purification. MS ES⁺: 506.1.

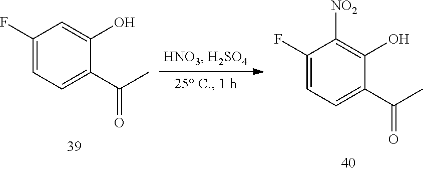

To a solution of H₂SO₄ (80 mL) was added 1-(4-fluoro-2-hydroxy-phenyl) ethanone 39 (20 g, 129.75 mmol, 1 eq) at 0° C. HNO₃ (15.63 g, 168.68 mmol, 11.16 mL, 68% purity, 1.3 eq) was added to the mixture dropwise at 0° C. during 30 min. The mixture was stirred at 25° C. for 1 hr. TLC showed a new spot was formed. Ice-water (800 ml) was added. The mixture was extracted with DCM (800 mL×3). The combined organic layer was washed with brine (500 mL), dried over Na₂SO₄, and evaporated in vacuum to give the residue. The residue was purified by flash silica gel chromatography (ISCO®; 120 g SepaFlash® Silica Flash Column, Eluent of 0˜10% Ethyl acetate/Petroleum ether gradient @ 80 mL/min) to give the nitro derivative 40 (13.4 g, crude) as a yellow solid.

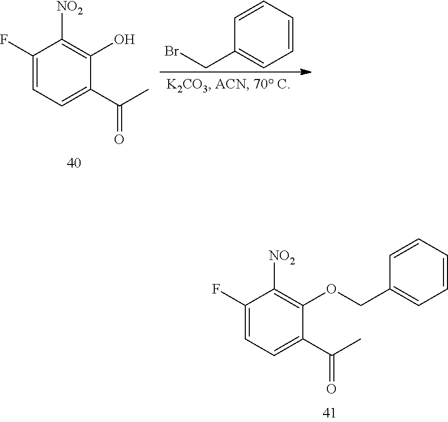 [SMILES: CC(=O)c1ccc(F)c([N+](=O)[O-])c1O ; BrCc1ccccc1 ; CC(=O)c1ccc(F)c([N+](=O)[O-])c1OCc1ccccc1]

To a solution of 1-(4-fluoro-2-hydroxy-3-nitro-phenyl) ethanone 40 (13.4 g, 67.29 mmol, 1 eq), K₂CO₃ (18.60 g, 134.58 mmol, 2 eq), bromomethylbenzene (12.66 g, 74.02 mmol, 8.79 mL, 1.1 eq) in MeCN (130 mL). The mixture was stirred at 70° C. for 8 hr. TLC showed a new spot was formed. The reaction mixture was concentrated under reduced pressure to give a residue. The residue was purified by flash silica gel chromatography (ISCO®; 80 g SepaFlash® Silica Flash Column, Eluent of 0˜10% Ethyl acetate/Petroleum ether gradient @ 65 mL/min) to give benzyl ether 41 (11.5 g, 39.76 mmol, 59.08% yield) as a yellow solid. ¹H NMR (400 MHZ, DMSO-d₆) δ=8.07 (dd, J=6.4, 8.9 Hz, 1H), 7.54 (t, J=9.0 Hz, 1H), 7.45-7.33 (m, 5H), 5.06 (s, 2H), 2.61 (s, 3H).

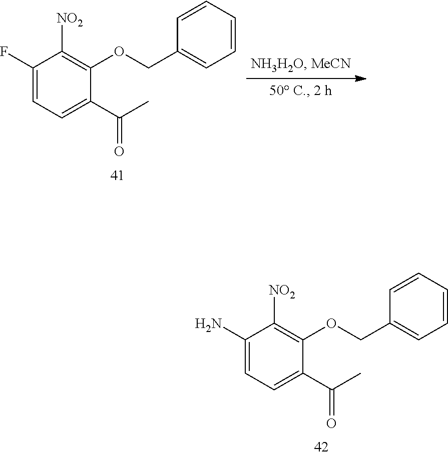 [SMILES: CC(=O)c1ccc(F)c([N+](=O)[O-])c1OCc1ccccc1 ; CC(=O)c1ccc(N)c([N+](=O)[O-])c1OCc1ccccc1]

To a solution of 1-(2-benzyloxy-4-fluoro-3-nitro-phenyl) ethanone 41 (11.5 g, 39.76 mmol, 1 eq) in MeCN (100 mL) was added NH₃·H₂O (83.60 g, 596.35 mmol, 91.87 mL, 25% purity, 15 eq) dropwise at 25° C. The reaction was stirred at 50° C. for 2 hr. LCMS showed the formation of desired product. The reaction mixture was concentrated under reduced pressure to give the amine 42 (8.5 g, crude) as a yellow oil which was used for next step without further purification. MS ES⁺: 286.9. ¹H NMR (400 MHZ, DMSO-d₆) δ=7.69 (d, J=9.0 Hz, 1H), 7.48-7.30 (m, 5H), 6.82 (s, 2H), 6.70 (d, J=9.1 Hz, 1H), 4.94 (s, 2H), 2.46 (s, 3H).

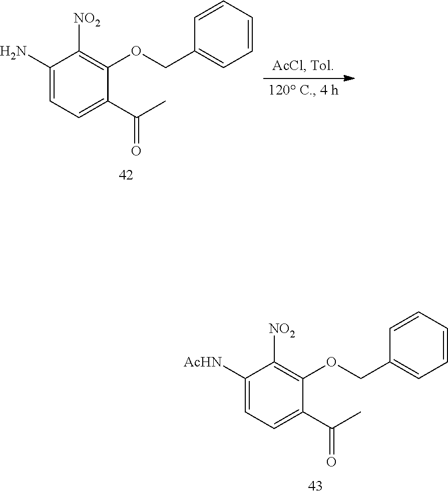 [SMILES: CC(=O)c1ccc(N)c([N+](=O)[O-])c1OCc1ccccc1 ; CC(=O)Nc1ccc(C(C)=O)c(OCc2ccccc2)c1[N+](=O)[O-]]

To a solution of 1-(4-amino-2-benzyloxy-3-nitro-phenyl) ethanone 42 (8.5 g, 29.69 mmol, 1 eq) and acetyl chloride (2.56 g, 32.66 mmol, 2.33 mL, 1.1 eq) was added Tol. (80 mL). The mixture was stirred at 120° C. for 8 hr. LCMS showed the formation of desired product. The reaction mixture was concentrated under reduced pressure to give acetamide 43 (9 g, crude) as a yellow oil which was used for next step without further purification. MS ES⁺: 329.3 ¹H NMR (400 MHZ, CDCl₃) δ=13.74 (s, 1H), 8.94 (br s, 1H), 8.09 (d, J=9.1 Hz, 1H), 7.88 (d, J=9.1 Hz, 1H), 7.43-7.30 (m, 3H), 7.09 (s, 3H), 2.66 (s, 3H), 2.27 (s, 3H).

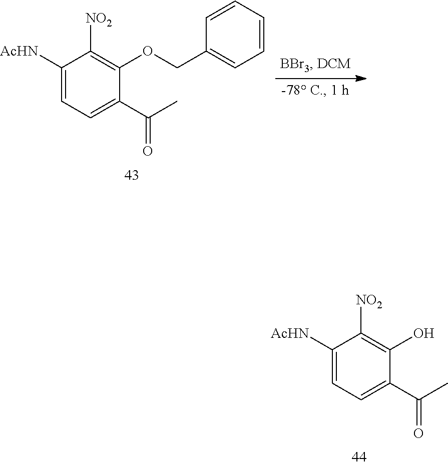 [SMILES: CC(=O)Nc1ccc(C(C)=O)c(OCc2ccccc2)c1[N+](=O)[O-] ; CC(=O)Nc1ccc(C(C)=O)c(O)c1[N+](=O)[O-]]

To a solution of N-(4-acetyl-3-benzyloxy-2-nitro-phenyl) acetamide 43 (9 g, 27.41 mmol, 1 eq) in DCM (90 mL) was added a solution of BBr₃ (1 M, 32.89 mL, 1.2 eq) drop-wise at −78° C. under N₂. The reaction mixture was stirred at −78° C. for 1 hr. LCMS showed the formation of desired product. Ice-water (1500 ml) was added. The mixture was extracted with DCM (1000 mL×3). The combined organic layer was washed with brine (500 mL), dried over Na₂SO₄, and evaporated in vacuum to give the residue. The residue was purified by flash silica gel chromatography (ISCO®; 80 g SepaFlash® Silica Flash Column, Eluent of 0˜20% Ethyl acetate/Petroleum ether gradient @ 80 mL/min) to give hydroxy acetamide 44 (5.5 g, 23.09 mmol, 84.23% yield) as a yellow solid. MS ES⁺: 239.0; ¹H NMR (400 MHZ, DMSO-d₆) δ=13.30 (s, 1H), 10.40 (s, 1H), 8.21 (d, J=8.9 Hz, 1H), 7.36 (d, J=8.9 Hz, 1H), 2.74 (s, 3H), 2.16 (s, 3H).

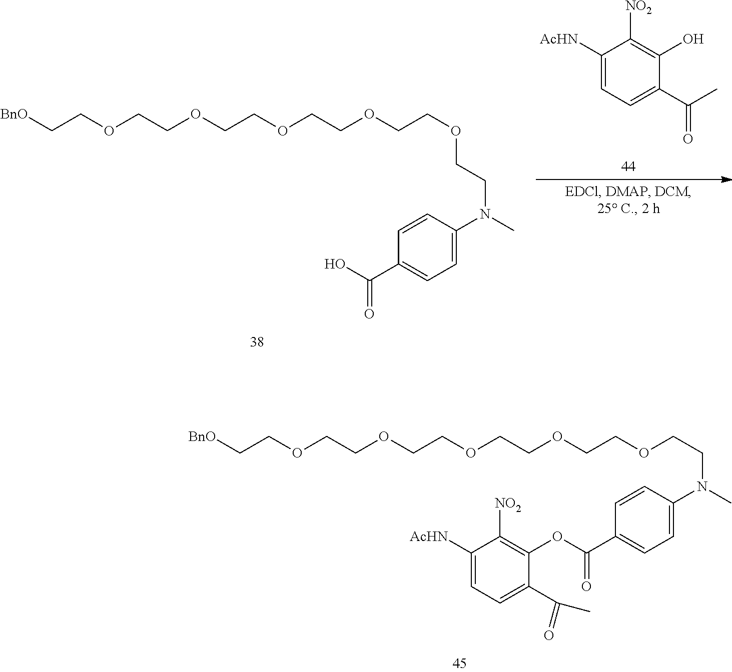 [SMILES: CN(CCOCCOCCOCCOCCOCCOCc1ccccc1)c1ccc(C(=O)O)cc1 ; CC(=O)Nc1ccc(C(C)=O)c(O)c1[N+](=O)[O-] ; CC(=O)Nc1ccc(C(C)=O)c(OC(=O)c2ccc(N(C)CCOCCOCCOCCOCCOCCOCc3ccccc3)cc2)c1[N+](=O)[O-]]

To a solution of 4-[2-[2-[2-[2-[2-(2-benzyloxyethoxy)ethoxy]ethoxy]ethoxy]ethoxy]ethyl-methyl-amino]benzoic acid 38 (1.4 g, 2.77 mmol, 1 eq) in DCM (10 mL) was added EDCI (1.06 g, 5.54 mmol, 2 eq), DMAP (67.66 mg, 553.80 μmol, 0.2 eq) and N-(4-acetyl-3-hydroxy-2-nitro-phenyl) acetamide 44 (659.56 mg, 2.77 mmol, 1 eq). The mixture was stirred at 25° C. for 2 hr. LC-MS showed the starting material was consumed completely and one main peak with desired m/z was detected. The reaction mixture was concentrated under reduced pressure, resulting residue was purified by flash silica gel chromatography (ISCO®; 20 g SepaFlash® Silica Flash Column, Eluent of 0˜100% Ethyl acetate/Petroleum ether gradient @ 100 mL/min) to give the ester 45 (1 g, 1.38 mmol, 49.76% yield) as a yellow oil. MS ES⁺: 726.6 ¹H NMR (400 MHZ, CDCl₃) δ=8.75 (s, 1H), 8.46 (d, J=9.0 Hz, 1H), 8.01 (dd, J=6.2, 8.9 Hz, 3H), 7.34 (d, J=4.5 Hz, 4H), 6.75-6.71 (m, 2H), 4.57 (s, 2H), 3.67-3.62 (m, 24H), 3.11 (s, 3H), 2.53 (s, 3H), 2.26 (s, 3H).

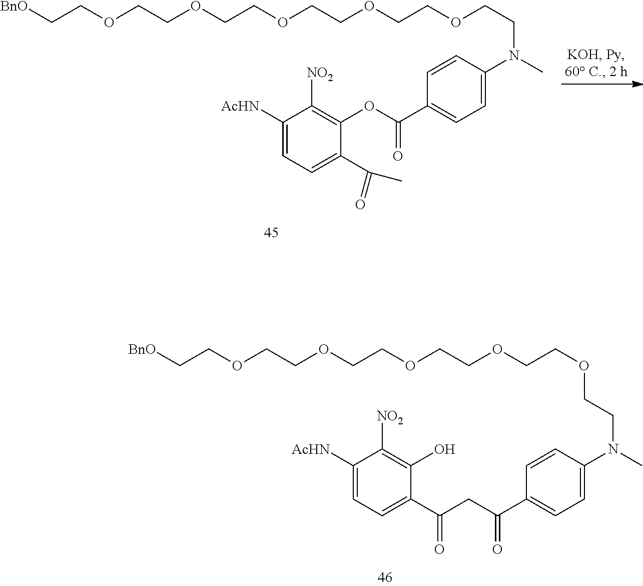 [SMILES: CC(=O)Nc1ccc(C(C)=O)c(OC(=O)c2ccc(N(C)CCOCCOCCOCCOCCOCCOCc3ccccc3)cc2)c1[N+](=O)[O-] ; CC(=O)Nc1ccc(C(=O)CC(=O)c2ccc(N(C)CCOCCOCCOCCOCCOCCOCc3ccccc3)cc2)c(O)c1[N+](=O)[O-]]

To a solution of (3-acetamido-6-acetyl-2-nitro-phenyl) 4-[2-[2-[2-[2-[2-(2-benzyloxyethoxy)ethoxy]ethoxy]ethoxy]ethoxy]ethylmethyl-amino]benzoate 45 (1 g, 1.38 mmol, 1 eq) in Pyridine (15 mL) was added KOH (386.52 mg, 6.89 mmol, 5 eq). The mixture was stirred at 60° C. for 2 hr. LC-MS showed the start material was consumed completely and one main peak with desired m/z was detected. The reaction mixture was cooled to room temperature. The reaction mixture was acidized by 1N HCl until pH=5-6, and then diluted with H₂O (20 mL) and extracted with EA (20 mL×3). The combined organic layers were washed with brine (40 mL×1), dried over anhydrous Na₂SO₄, filtered, and concentrated under reduced pressure to give the title compound 46 (1.3 g, crude) as a yellow oil which was used into the next step without further purification. MS ES⁺: 726.2.

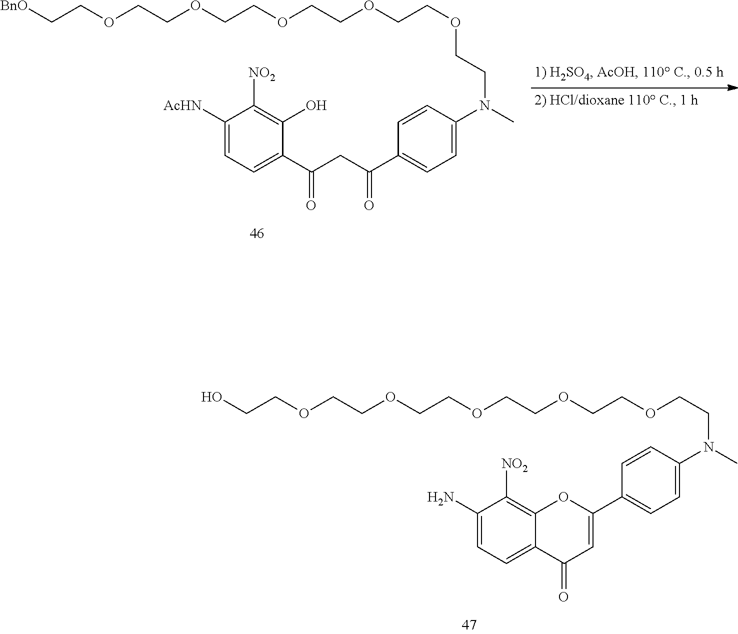 [SMILES: CC(=O)Nc1ccc(C(=O)CC(=O)c2ccc(N(C)CCOCCOCCOCCOCCOCCOCc3ccccc3)cc2)c(O)c1[N+](=O)[O-] ; CN(CCOCCOCCOCCOCCOCCO)c1ccc(-c2cc(=O)c3ccc(N)c([N+](=O)[O-])c3o2)cc1]

To a solution of N-[4-[3-[4-[2-[2-[2-[2-[2-(2-benzyloxyethoxy)ethoxy]ethoxy]ethoxy]ethoxy]ethyl-methyl-amino]phenyl]-3-oxo-propanoyl]-3-hydroxy-2-nitro-phenyl]acetamide 46 (1.3 g, 1.79 mmol, 1 eq) in AcOH (20 mL) was added H₂SO₄ (175.68 mg, 1.79 mmol, 95.48 μL, 1 eq). The mixture was stirred at 110° C. for 0.5 hr. LC-MS showed the starting material was consumed completely and one main peak with desired m/z was detected. The reaction mixture was cooled to room temperature. The reaction mixture was neutralized by aq. NaHCO₃ until pH=7-8, then extracted with EA (20 mL×3). The combined organic layers were washed with brine (40 mL×1), dried over anhydrous Na₂SO₄, filtered, concentrated under reduced pressure, resulting residue was dissolved in dioxane (20 mL) and HCl (20 mL). The mixture was stirred at 110° C. for 1 hr. LC-MS showed the start material was consumed completely and one main peak with desired m/z was detected. The reaction mixture was concentrated under reduced pressure, to give the title compound 47 (1 g, crude) as a brown gum which was used into the next step without further purification. MS ES⁺: 576.3.

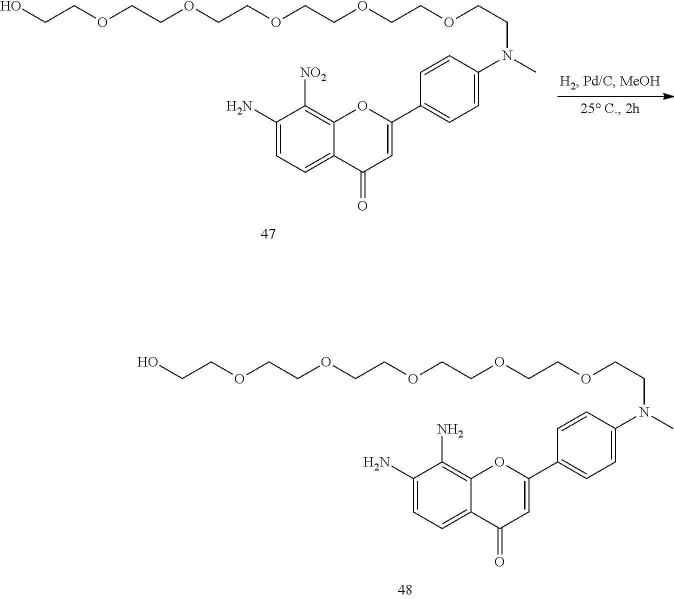 [SMILES: CN(CCOCCOCCOCCOCCOCCO)c1ccc(-c2cc(=O)c3ccc(N)c([N+](=O)[O-])c3o2)cc1 ; CN(CCOCCOCCOCCOCCOCCO)c1ccc(-c2cc(=O)c3ccc(N)c(N)c3o2)cc1]

To a solution of 7-amino-2-[4-[2-[2-[2-[2-[2-(2-hydroxyethoxy)ethoxy]ethoxy]ethoxy]ethoxy]ethyl-methyl-amino]phenyl]-8-nitro-chromen-4-one 47 (1 g, 1.74 mmol, 1 eq) in MeOH (30 mL) was added Pd/C (10%, 0.5 g) under N₂ atmosphere. The suspension was degassed and purged with H₂ for 3 times. The mixture was stirred under H₂ (15 Psi) at 25° C. for 2 hr. LC-MS showed the starting material was consumed completely and one main peak with desired m/z was detected. The reaction mixture was filtered and concentrated under reduced pressure to give the title compound 48 (950 mg, crude) as a yellow gum which was used into the next step without further purification. MS ES⁺: 546.2.

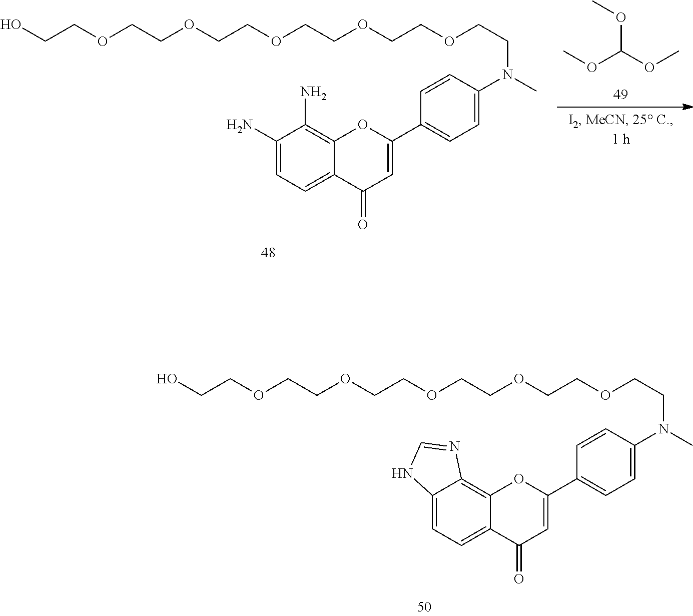

To a solution of 7,8-diamino-2-[4-[2-[2-[2-[2-[2-(2-hydroxyethoxy)ethoxy]ethoxy]ethoxy]ethoxy]ethyl-methylamino]phenyl]chromen-4-one 48 (850 mg, 1.56 mmol, 1 eq) in MeCN (20 mL) was added trimethoxymethane 49 (181.85 mg, 1.71 mmol, 187.86 uL, 1.1 eq) and I₂ (39.54 mg, 155.79 μmol, 0.1 eq). The mixture was stirred at 25° C. for 1 hr. LC-MS showed the starting material was consumed completely and one main peak with desired m/z was detected. The reaction mixture was quenched by addition aq. NaHSO₃ (20 mL), and then extracted with EA (20 mL×3). The combined organic layers were washed with brine (40 mL×1), dried over anhydrous Na₂SO₄, filtered, and concentrated under reduced pressure, resulting residue was purified by flash silica gel chromatography (ISCO®; 20 g SepaFlash® Silica Flash Column, Eluent of 0˜15% MeOH/DCM gradient @ 100 mL/min) to give the title compound 50 (500 mg, 899.90 μmol, 57.77% yield) as a red gum. MS ES⁺: 556.3 ¹H NMR (400 MHZ, DMSO-d₆) δ=8.71 (br s, 1H), 8.04 (br d, J=6.9 Hz, 2H), 7.87 (br d, J=4.5 Hz, 1H), 7.66 (br s, 1H), 7.55-7.00 (m, 1H), 6.86 (br d, J=8.1 Hz, 3H), 3.62 (br s, 4H), 3.47 (br s, 18H), 3.38 (br s, 2H), 3.16 (s, 1H), 3.05 (br s, 3H).

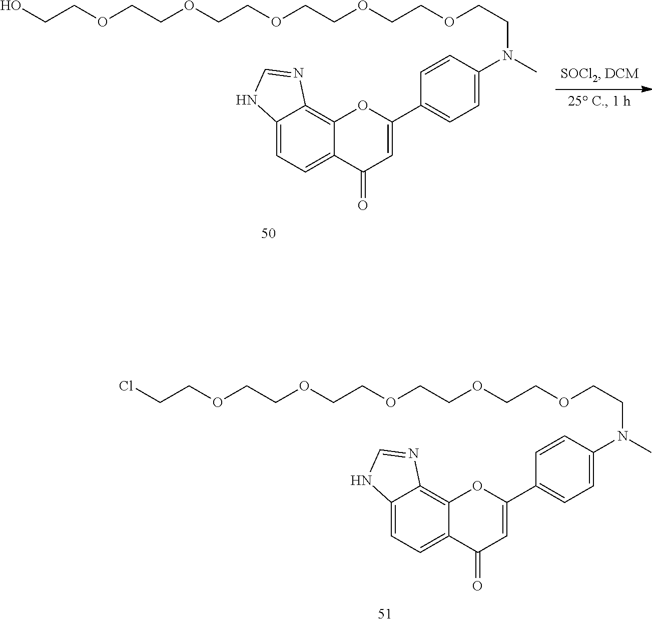 [SMILES: CN(CCOCCOCCOCCOCCOCCO)c1ccc(-c2cc(=O)c3ccc4[nH]cnc4c3o2)cc1 ; CN(CCOCCOCCOCCOCCOCCCl)c1ccc(-c2cc(=O)c3ccc4[nH]cnc4c3o2)cc1]

To a solution of 8-[4-[2-[2-[2-[2-[2-(2-hydroxyethoxy)ethoxy]ethoxy]ethoxy]ethoxy]ethyl-methyl-amino]phenyl]-3Hpyrano[2,3-e]benzimidazol-6-one 50 (450 mg, 809.91 μmol, 1 eq) in DCM (20 mL) was added SOCI₂ (8.10 mmol, 587.53 μL, 10 eq). The mixture was stirred at 25° C. for 1 hr. LC-MS showed the start material was consumed completely and one main peak with desired m/z was detected. The reaction mixture was poured into saturated NaHCO₃ aq. (30 mL) and extracted with EA (30 mL×3). The combined organic layers were washed with brine (60 mL×1), dried over anhydrous Na₂SO₄, filtered, and concentrated under reduced pressure, resulting residue was purified by flash silica gel chromatography (ISCO®; 12 g SepaFlash®), using 0-10% MeOH/DCM gradient @ 80 mL/min) to give the title compound 51 as a mixture of tautomers (350 mg, 559.08 μmol, 69.03% yield, 91.7% purity) as a brown gum. MS ES⁺: 574.1 ¹H NMR (400 MHz, DMSO-d₆) δ=9.56 (s, 1H), 8.16 (d, J=8.9 Hz, 2H), 8.03 (d, J=8.6 Hz, 1H), 7.79 (d, J=8.6 Hz, 1H), 6.93 (s, 1H), 6.84 (d, J=9.0 Hz, 2H), 3.70-3.67 (m, 2H), 3.66-3.58 (m, 6H), 3.55-3.46 (m, 16H), 3.06 (s, 3H).

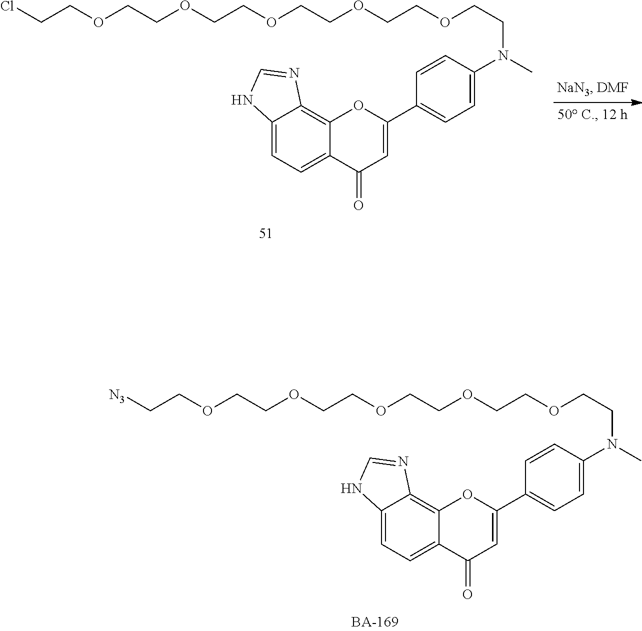 [SMILES: CN(CCOCCOCCOCCOCCOCCCl)c1ccc(-c2cc(=O)c3ccc4[nH]cnc4c3o2)cc1 ; CN(CCOCCOCCOCCOCCOCCN=[N+]=[N-])c1ccc(-c2cc(=O)c3ccc4[nH]cnc4c3o2)cc1]

Flavone PEG-5 chloride 51 (180 mg, 0.314 mmol, 1 eq), and sodium azide (102 mg, 1.568 mmol, 5 eq) in dry DMF (1 mL) was stirred for 12 h at 70° C., Reaction mixture was cooled and diluted with water 10 mL, extracted with DCM 50 mL, dried over Na₂SO₄ and evaporated, resulting crude was purified by column chromatography using 0-20% MeOH/DCM, pure fractions were combined and concentrated to obtain azide BA-169 (28:72 tautomeric mixture)(50 mg, 25%) as a yellow gum. LCMS m/z=681 (M⁺+1) and NMR are corresponding with product. >95% purity by LCMS. ¹H NMR (499 MHz, DMSO-d₆) δ 13.09 (s, 1H), 8.43 (s, 1H), 8.12 (d, J=8.9 Hz, 1H), 7.96 (d, J=9.1 Hz, 2H), 7.85 (d, J=8.6 Hz, 1H), 7.59 (d, J=8.5 Hz, 1H), 6.91-6.81 (m, 2H), 6.83 (s, 1H), 3.67-3.60 (m, 3H), 3.58-3.54 (m, 2H), 3.54-3.47 (m, 18H), 3.36 (m, 2H), 3.05 (s, 3H).

BA-169 was conjugated to an oligo sense strand according to general procedure Type II. MW calculated: 8292.05. MW Found: 8291.14.

### Example 14: BA-170 and BA-201 Conjugates

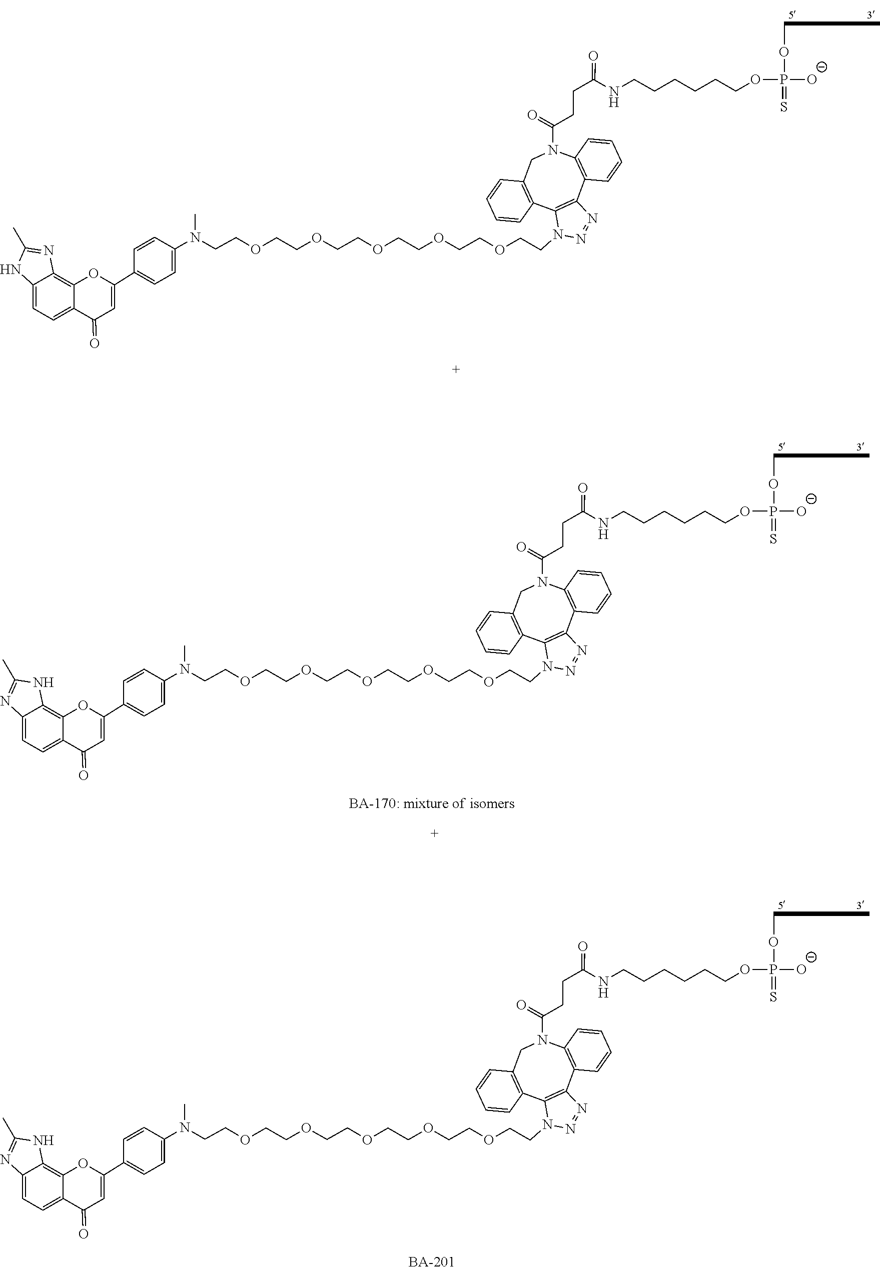

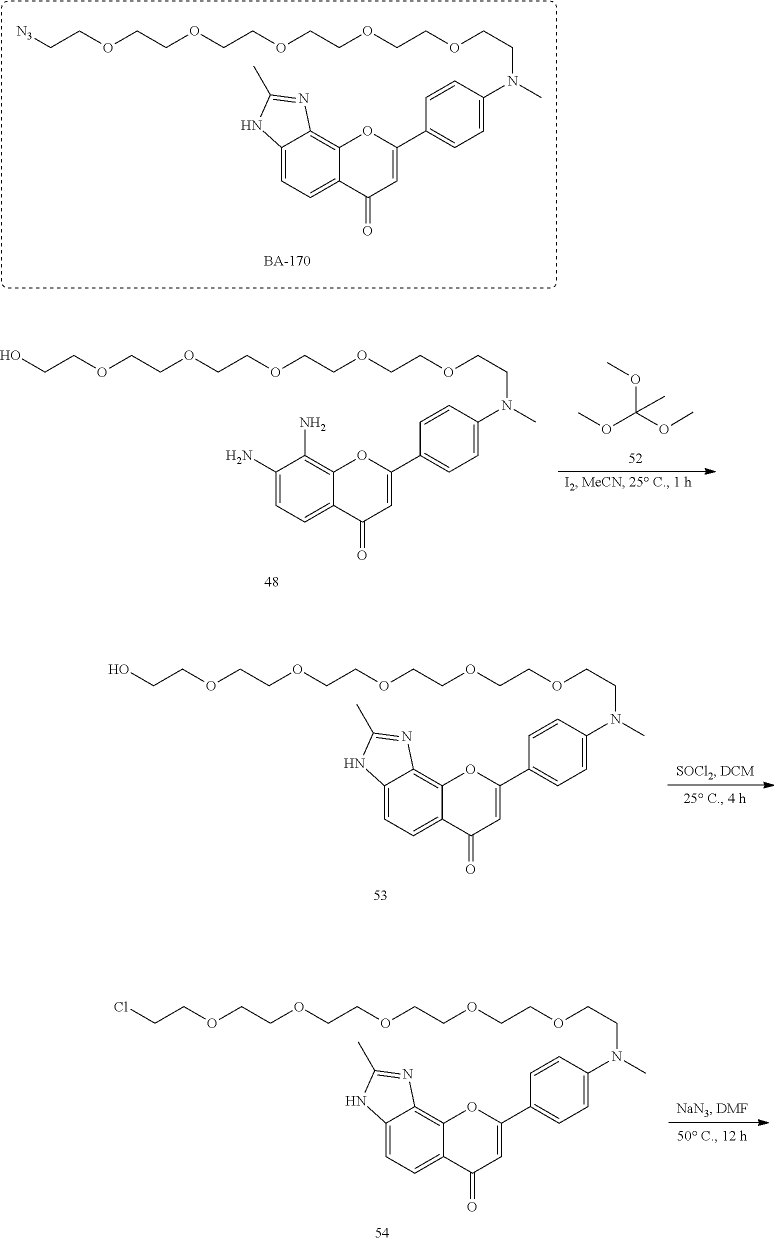

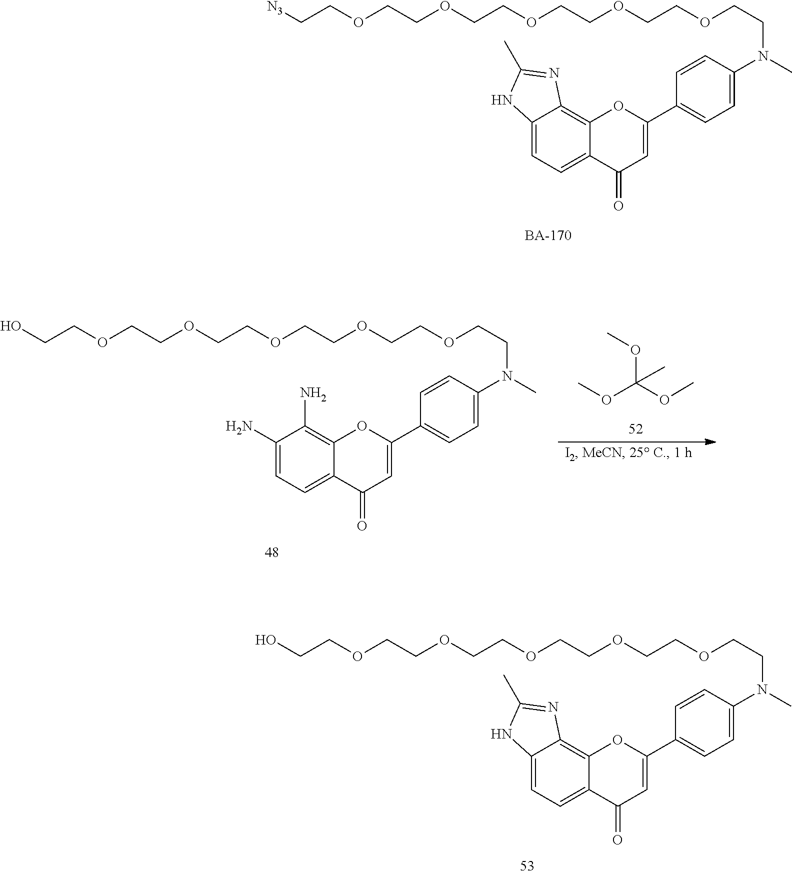

To a solution of 7,8-diamino-2-[4-[2-[2-[2-[2-[2-(2-hydroxyethoxy) ethoxy]ethoxy]ethoxy]ethoxy]ethyl-methylamino]phenyl]chromen-4-one 48 (700 mg, 1.28 mmol, 1 eq) in MeCN (15 mL) was added 1,1,1-trimethoxyethane 52 (169.55 mg, 1.41 mmol, 1.1 eq) and I₂ (32.56 mg, 128.29 μmol, 25.84 μL, 0.1 eq). The mixture was stirred at 25° C. for 1 hr. LC-MS showed the starting material was consumed completely and one main peak with desired m/z was detected. The reaction mixture was quenched by addition aq. NaHSO₃ (20 mL), and then extracted with EA (20 mL×3). The combined organic layers were washed with brine (40 mL×1), dried over anhydrous Na₂SO₄, filtered, and concentrated under reduced pressure, resulting residue was purified by flash silica gel chromatography (ISCO®; 20 g SepaFlash® Silica Flash Column, Eluent of 0˜15% MeOH/DCM gradient @ 100 mL/min) to give the title compound 53 (470 mg, 825.08 umol, 64.31% yield) as a brown gum. MS ES⁺: 570.2

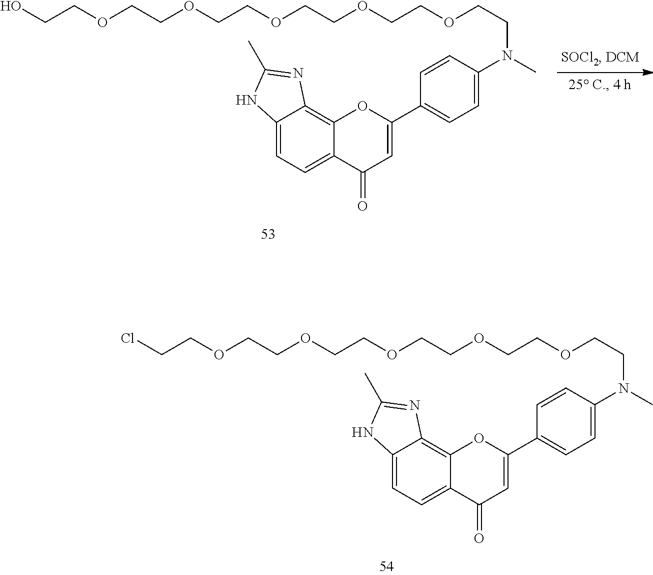 [SMILES: Cc1nc2c(ccc3c(=O)cc(-c4ccc(N(C)CCOCCOCCOCCOCCOCCO)cc4)oc32)[nH]1 ; Cc1nc2c(ccc3c(=O)cc(-c4ccc(N(C)CCOCCOCCOCCOCCOCCCl)cc4)oc32)[nH]1]

To a solution of 8-[4-[2-[2-[2-[2-[2-(2-hydroxyethoxy)ethoxy]ethoxy]ethoxy]ethoxy]ethyl-methyl-amino]phenyl]-2-methyl-3Hpyrano[2,3-e]benzimidazol-6-one 53 (400 mg, 702.19 umol, 1 eq) in DCM (6 mL) was added SOCI₂ (835.40 mg, 7.02 mmol, 509.39 μL, 10 eq). The mixture was stirred at 25° C. for 4 hr. LC-MS showed the start material was consumed completely and one main peak with desired m/z was detected. The reaction mixture was poured into saturated aq. NaHCO₃ (40 mL) and extracted with EA (40 mL×3). The combined organic layers were washed with brine (80 mL×1), dried over anhydrous Na₂SO₄, filtered, and concentrated under reduced pressure to give the title compound 54 as a mixture of tautomers (320 mg, 496.25 μmol, 70.67% yield, 91.2% purity) as a brown gum. MS ES⁺: 588.3 ¹H NMR (400 MHZ, DMSO-d₆) δ=8.08-7.85 (m, 2H), 7.76 (d, J=8.5 Hz, 1H), 7.50 (br d, J=8.3 Hz, 1H), 6.87 (br d, J=8.9 Hz, 2H), 6.79 (s, 1H), 3.70-3.66 (m, 2H), 3.66-3.60 (m, 6H), 3.54-3.47 (m, 16H), 3.05 (s, 3H), 2.61 (s, 3H).

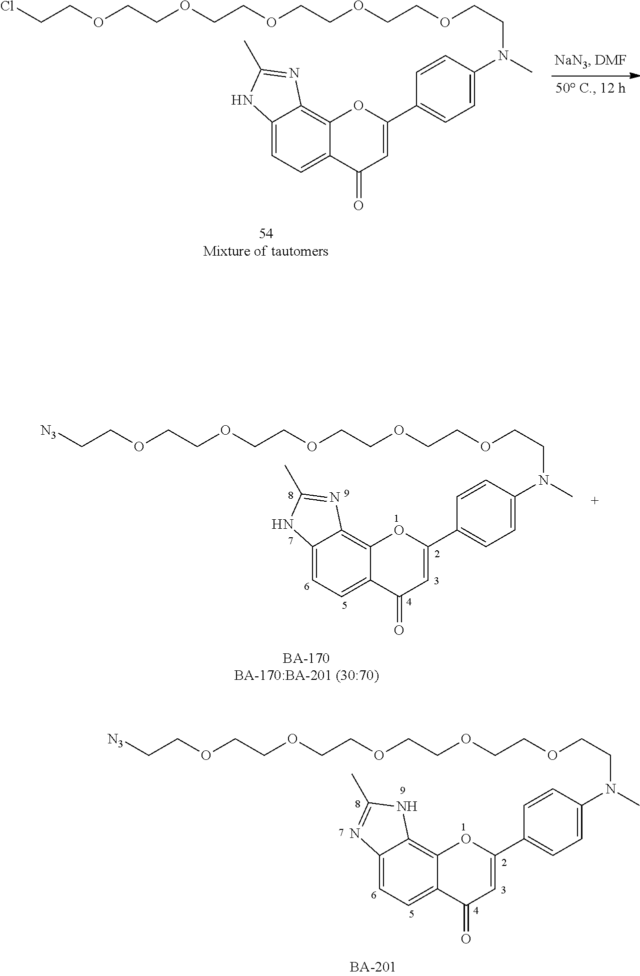 [SMILES: Cc1nc2c(ccc3c(=O)cc(-c4ccc(N(C)CCOCCOCCOCCOCCOCCCl)cc4)oc32)[nH]1 ; Cc1nc2c(ccc3c(=O)cc(-c4ccc(N(C)CCOCCOCCOCCOCCOCCN=[N+]=[N-])cc4)oc32)[nH]1 ; Cc1nc2ccc3c(=O)cc(-c4ccc(N(C)CCOCCOCCOCCOCCOCCN=[N+]=[N-])cc4)oc3c2[nH]1]

Flavone PEG-5 chloride 54 (310 mg, 0.527 mmol, 1 eq), and sodium azide (171 mg, 2.63 mmol, 5 eq) in dry DMF (2 mL) was stirred for 12 h at 70° C., Reaction mixture was cooled and diluted with water 20 mL, extracted with DCM 100 mL, dried over Na₂SO₄ and evaporated, resulting crude was purified by column chromatography using 0-20% MeOH/DCM, pure fractions were combined and concentrated to obtain azide BA-170 as a mixture of tautomers BA-170: BA-201 (30:70)(120 mg, 38%) as a dark orange gum. LCMS m/z=595 (M+1) and NMR are corresponding with product. >95% purity by LCMS. Tautomeric structures were confirmed by ¹H NMR. For BA-170 isomer wherein the 7-NH proton appears more down field at δ 13.38, whereas 9-NH proton in BA-201 appears up field at δ 12.98 compared to 7-NH. BA-170 ¹H NMR: (499 MHz, DMSO-d₆) δ 13.38, 12.85 (2s, 1H), 8.09, 7.94 (d, J=8.7 Hz, 2H), 7.77, 7.72 (d, J=8.5 Hz, 1H), 7.54, 7.47 (d, J=8.5 Hz, 1H), 6.88, 6.85 (d, J=9.0 Hz, 2H), 6.81, 6.79 (s, 1H), 3.67-3.62 (m, 2H), 3.58-3.54 (m, 2H), 3.54-3.45 (m, 16H), 3.38-3.35 (m, 2H), 3.06, 3.05 (s, 3H), 2.63, 2.60 (s, 3H).

BA-170 tautomeric mixture (80 mg) was further purified by flash column chromatography to obtain pure BA-201 50 mg as a dark orange gum.

BA-201 NMR: ¹H NMR (499 MHz, DMSO-d₆) δ 12.98 (s, 1H), 7.98 (d, J=8.7 Hz, 2H), 7.76 (d, J=8.5 Hz, 1H), 7.50 (d, J=8.5 Hz, 1H), 6.87 (d, J=9.0 Hz, 2H), 6.80 (s, 1H), 3.62 (m, 4H), 3.58-3.54 (m, 2H), 3.54-3.45 (m, 16H), 3.40-3.34 (m, 2H), 3.05 (s, 3H), 2.61 (s, 3H).

BA-170 and BA-201 were independently conjugated to an oligo sense strand according to general procedure Type II. MW calculated: 8306.08. MW Found: 8304.43.

### Example 15: BA-173 Conjugate

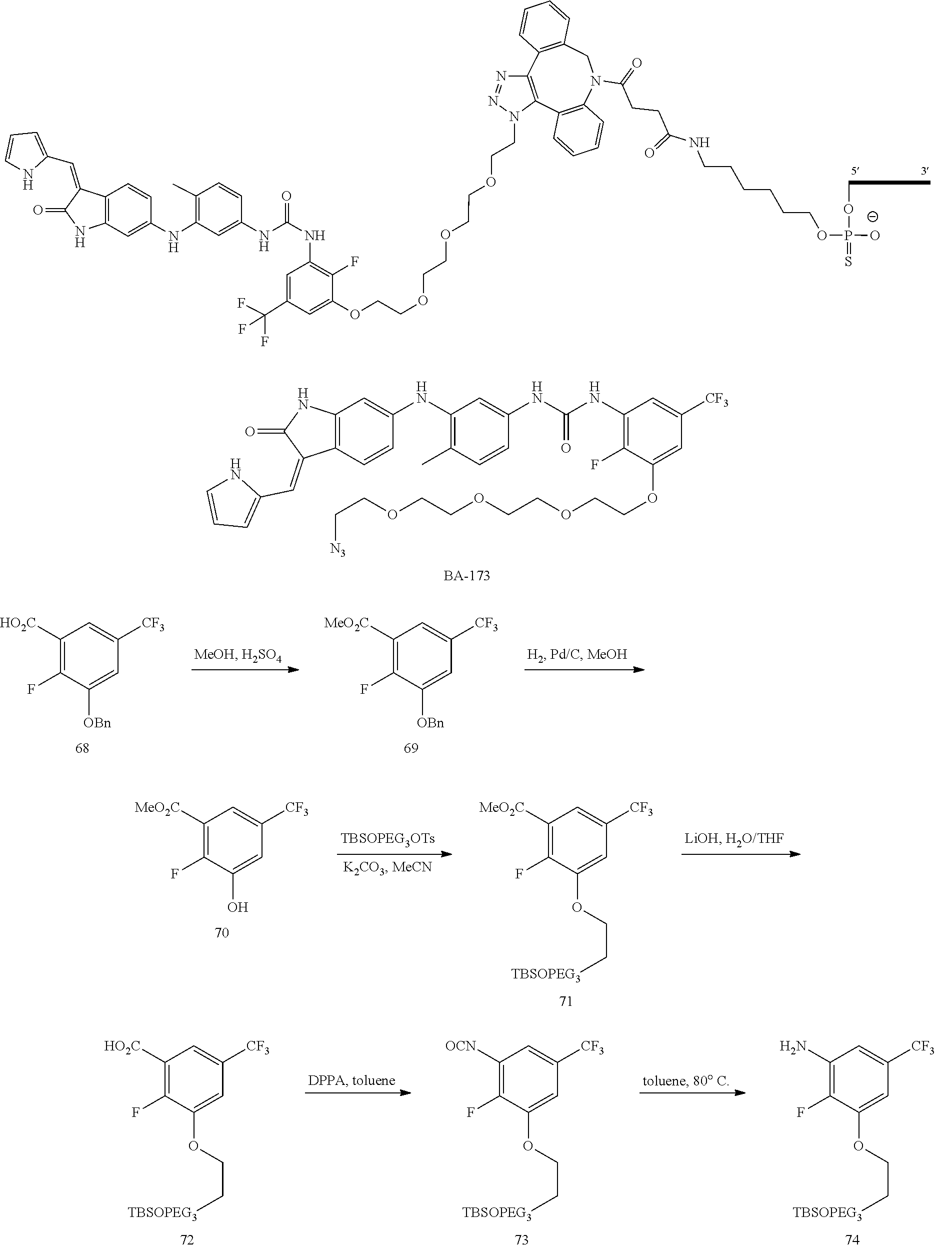

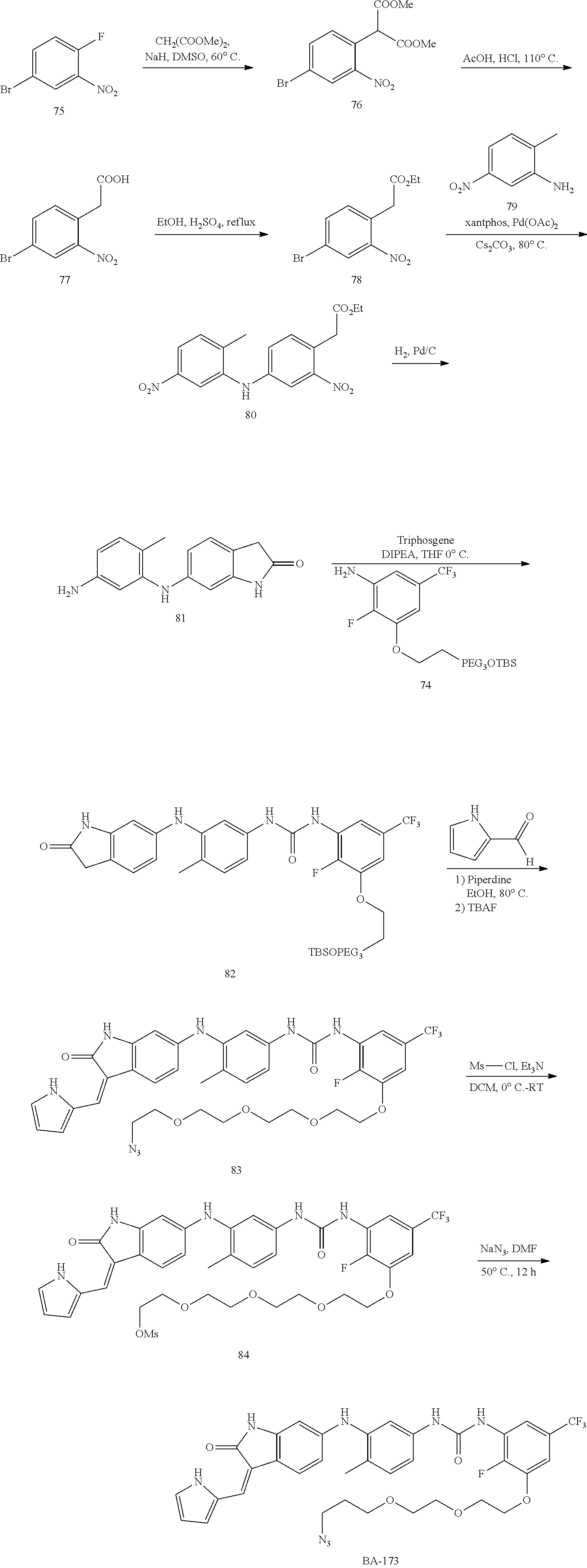

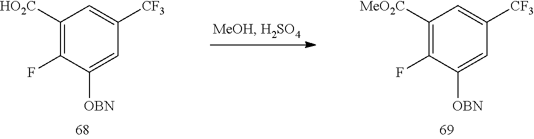

A solution of 3-(benzyloxy)-2-fluoro-5-(trifluoromethyl)benzoic acid 68 (4.9 g, 15.593 mmol, 1 equiv) in MeOH (49.00 mL, 1210.243 mmol, 77.61 equiv) was treated with two drop of H₂SO₄ (305.85 mg, 3.119 mmol, 0.2 equiv) at room temperature under nitrogen atmosphere. The resulting mixture was stirred for overnight at room temperature under nitrogen atmosphere. The reaction was quenched by the addition of NaHCO₃(sat) at room temperature. The resulting mixture was extracted with EA (3×20 mL). The combined organic layers were washed with NaCl (3×20 mL), dried over anhydrous Na₂SO₄. After filtration, the filtrate was concentrated under reduced pressure. The crude product methyl 3-(benzyloxy)-2-fluoro-5-(trifluoromethyl)benzoate 69 (5 g, crude) was used in the next step directly without further purification. LCMS-(M+H)⁺: 329. ¹H NMR (400 MHZ, Chloroform-d) δ 7.79 (dd, J=5.5, 2.3, 0.9 Hz, 1H), 7.48-7.32 (m, 6H), 5.19 (s, 2H), 3.96 (s, 3H).

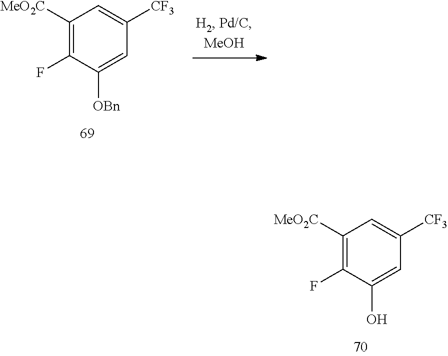 [SMILES: COC(=O)c1cc(C(F)(F)F)cc(OCc2ccccc2)c1F ; COC(=O)c1cc(C(F)(F)F)cc(O)c1F]

A mixture of methyl 3-(benzyloxy)-2-fluoro-5-(trifluoromethyl)benzoate 69 (5 g, 15.232 mmol, 1 equiv) and Pd/C (1.51 g, 14.189 mmol, 0.93 equiv) in MeOH (150 mL) was stirred for 3 hours at room temperature under hydrogen atmosphere. The resulting mixture was filtered, the filter cake was washed with MeOH (3×30 mL). The filtrate was concentrated under reduced pressure. This resulted in methyl 2-fluoro-3-hydroxy-5-(trifluoromethyl)benzoate 70 (3.7 g, crude) as a white solid. LCMS: (M+H)⁺239.

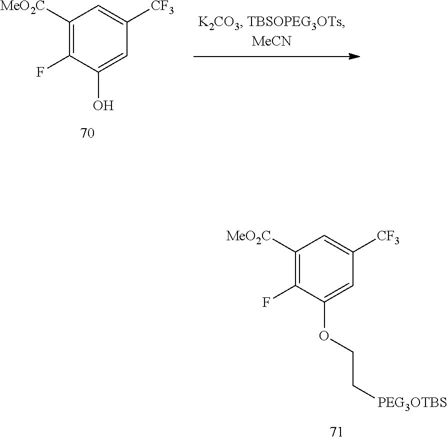

A solution/mixture of methyl 2-fluoro-3-hydroxy-5-(trifluoromethyl)benzoate 70 (3.7 g, 15.537 mmol, 1 equiv), K₂CO₃ (3.22 g, 23.306 mmol, 1.5 equiv) and TBSOPEG₃OTs (7.18 g, 15.537 mmol, 1.0 equiv) in MeCN (111.00 mL, 2111.634 mmol, 135.91 equiv) was stirred for overnight at 70° C. under nitrogen atmosphere. The reaction was quenched with water at room temperature, The resulting mixture was extracted with EA (3×50 mL). The combined organic layers were washed with NaCl (3×50 mL), dried over anhydrous Na₂SO₄. After filtration, the filtrate was concentrated under reduced pressure. The residue was purified by silica gel column chromatography, eluted with EA/PE (30%) to afford methyl 2-fluoro-3-[(2,2,3,3-tetramethyl-4,7,10,13-tetraoxa-3-silapentadecan-15-yl)oxy]-5-(trifluoromethyl)benzoate 71 (6 g, 58.44%) as a yellow oil. LCMS: (M+H)⁺529. ¹H NMR (400 MHZ, Chloroform-d) δ 7.83-7.77 (m, 1H), 7.43 (dd, J=7.0, 2.3 Hz, 1H), 4.32-4.25 (m, 2H), 3.98 (s, 3H), 3.93 (dd, J=5.5, 3.8 Hz, 2H), 3.82-3.68 (m, 4H), 3.72-3.65 (m, 6H), 3.57 (t, J=5.5 Hz, 2H), 0.91 (s, 9H), 0.08 (s, 6H).

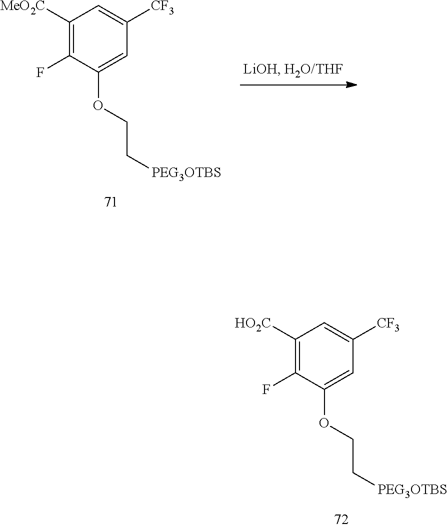

A solution of methyl 2-fluoro-3-[(2,2,3,3-tetramethyl-4,7,10,13-tetraoxa-3-silapentadecan-15-yl)oxy]-5-(trifluoromethyl)benzoate 71 (5.59 g, 10.575 mmol, 1 equiv) in THF (55 mL) was treated with LiOH in water (26.44 mL, 52.875 mmol, 5 equiv) dropwise under nitrogen atmosphere. The resulting mixture was stirred for 2 hr at room temperature under nitrogen atmosphere. The resulting mixture treated with AcOH (3.81 g, 63.450 mmol, 6 equiv) dropwise at room temperature. The resulting mixture was extracted with EA (3×100 mL). The combined organic layers were washed with NaCl (2×50 mL), dried over anhydrous Na₂SO₄. After filtration, the filtrate was concentrated under reduced pressure. The residue was purified by silica gel column chromatography, eluted with EA/PE (75%) to afford 2-fluoro-3-[(2,2,3,3-tetramethyl-4,7,10,13-tetraoxa-3-silapentadecan-15-yl)oxy]-5-(trifluoromethyl)benzoic acid 72 (4.0 g, 73.51%) as a yellow oil. LCMS: (M+H)+515.

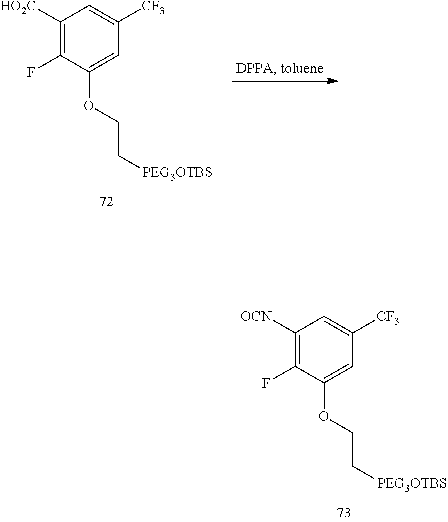

A solution/mixture of 2-fluoro-3-[(2,2,3,3-tetramethyl-4,7,10,13-tetraoxa-3-silapentadecan-15-yl)oxy]-5-(trifluoromethyl)benzoic acid 72 (1.5 g, 2.915 mmol, 1 equiv), TEA (324.47 mg, 3.207 mmol, 1.1 equiv) and DPPA (882.43 mg, 3.207 mmol, 1.1 equiv) in toluene (30 mL) was stirred for overnight at room temperature under nitrogen atmosphere. The reaction was quenched by the addition of water (5 mL) at room temperature. The resulting mixture was extracted with ethyl acetate (3×50 mL). The combined organic layers were washed with NaCl (3×50 mL), dried over anhydrous Na₂SO₄. After filtration, the filtrate was concentrated under reduced pressure. The residue was purified by silica gel column chromatography, eluted with EA/PE (˜30%) to afford 15-[2-fluoro-3-isocyanato-5-(trifluoromethyl) phenoxy]-2,2,3,3-tetramethyl-4,7,10,13-tetraoxa-3-silapentadecane 73 (740 mg) as a yellow oil. LCMS: (M+H)⁺512

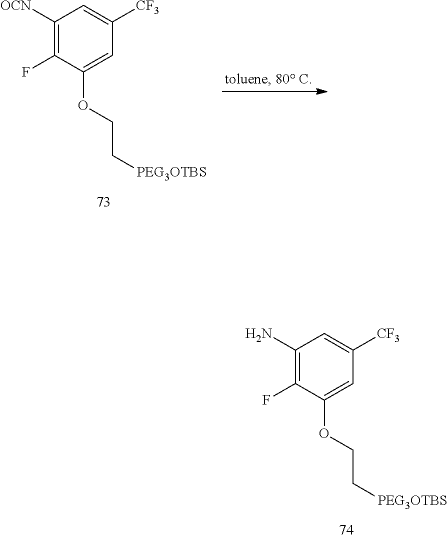

A solution/mixture of 15-[2-fluoro-3-isocyanato-5-(trifluoromethyl) phenoxy]-2,2,3,3-tetramethyl-4,7,10,13-tetraoxa-3-silapentadecane 73 (750 mg, 1.466 mmol, 1 equiv) in toluene (20.45 mL, 192.237 mmol, 131.13 equiv) was stirred for 2 hr at 80° C. under nitrogen atmosphere. The resulting mixture was concentrated under reduced pressure. This resulted in 2-fluoro-3-[(2,2,3,3-tetramethyl-4,7,10,13-tetraoxa-3-silapentadecan-15-yl)oxy]-5-(trifluoromethyl) aniline 74 (720 mg, crude) as a yellow oil. LCMS: (M+H)⁺486.

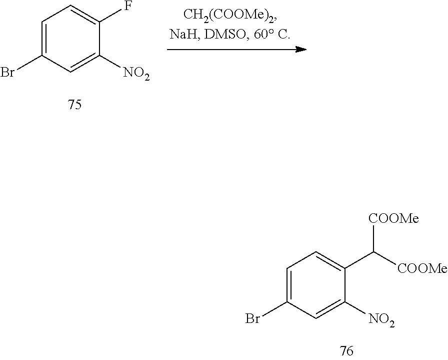 [SMILES: O=[N+]([O-])c1cc(Br)ccc1F ; COC(=O)C(C(=O)OC)c1ccc(Br)cc1[N+](=O)[O-]]

To a stirred mixture of 4-bromo-1-fluoro-2-nitrobenzene 75 (4 g, 18.182 mmol, 1 equiv) and 1,3-dimethyl propanedioate (3.60 g, 27.249 mmol, 1.50 equiv) in DMSO-d₆ (40 mL) was added NaH (872.67 mg, 21.818 mmol, 1.2 equiv, 60%) in portions at room temperature under nitrogen atmosphere. The resulting mixture was stirred for 2 h at 60° C. under nitrogen atmosphere. The reaction was quenched with sat. NH₄Cl (aq.)/ice at 0° C. The resulting mixture was extracted with EtOAc (3×100 mL). The combined organic layers were washed with water (4×40 mL), brine (2×40 mL) dried over anhydrous MgSO₄. After filtration, the filtrate was concentrated under reduced pressure. This resulted in 1,3-dimethyl 2-(4-bromo-2-nitrophenyl) propanedioate 76 (5.68 g, crude). LCMS: (M+H)⁺332.

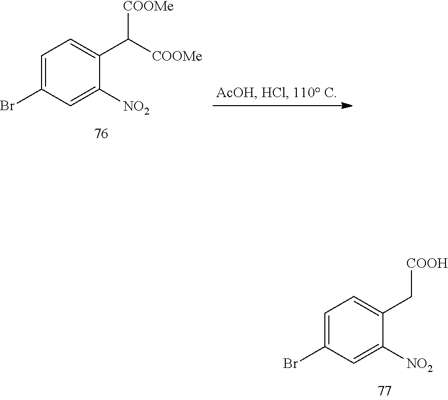 [SMILES: COC(=O)C(C(=O)OC)c1ccc(Br)cc1[N+](=O)[O-] ; O=C(O)Cc1ccc(Br)cc1[N+](=O)[O-]]

To a stirred solution of 1,3-dimethyl 2-(4-bromo-2-nitrophenyl) propanedioate 76 (5.68 g, 17.103 mmol, 1 equiv) in AcOH (57 mL) and HCl (57 mL) at room temperature under argon atmosphere. The resulting mixture was stirred for overnight at 110° C. under argon atmosphere. The mixture was allowed to cool down to room temperature. The resulting mixture was concentrated under reduced pressure. The product was precipitated by the addition of CH₂Cl₂. This resulted in (4-bromo-2-nitrophenyl) acetic acid 77 (2.9 g, 60.50%) as a grey solid. LCMS: (M+H)⁺260.

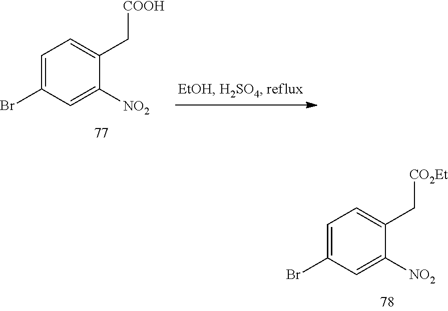 [SMILES: O=C(O)Cc1ccc(Br)cc1[N+](=O)[O-] ; CCOC(=O)Cc1ccc(Br)cc1[N+](=O)[O-]]

A solution/mixture of (4-bromo-2-nitrophenyl) acetic acid 77 (2.9 g, 10.729 mmol, 1 equiv) and H₂SO₄ (0.42 mL, 7.939 mmol, 0.74 equiv) in ethanol was stirred for 2 h at 85° C. under argon atmosphere. The reaction was quenched with NaHCO₃ at 0° C. The resulting mixture was extracted with EtOAc. The combined organic layers were washed with brine, dried over anhydrous Na₂SO₄. After filtration, the filtrate was concentrated under reduced pressure. This resulted in ethyl 2-(4-bromo-2-nitrophenyl)acetate 78 (2.9 g, 87.35%) as a brown oil. LCMS: (M+H)⁺288.

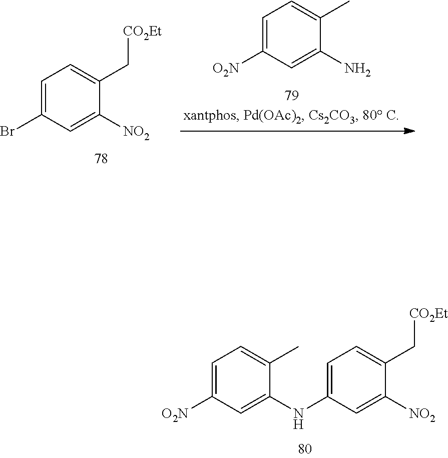 [SMILES: CCOC(=O)Cc1ccc(Br)cc1[N+](=O)[O-] ; Cc1ccc([N+](=O)[O-])cc1N ; CCOC(=O)Cc1ccc(Nc2cc([N+](=O)[O-])ccc2C)cc1[N+](=O)[O-]]

A solution of ethyl 2-(4-bromo-2-nitrophenyl)acetate 78 (2.9 g, 10.066 mmol, 1 equiv) and 2-amino-4-nitrotoluene 79 (2.30 g, 15.099 mmol, 1.5 equiv) in toluene was treated with Pd(OAc)₂ (0.23 g, 1.007 mmol, 0.1 equiv), xantphos (1.16 g, 2.013 mmol, 0.2 equiv) under nitrogen atmosphere followed by the addition of Cs₂CO₃ (8.20 g, 25.165 mmol, 2.5 equiv) in portions at room temperature. The resulting was mixture stirred for 3 h at 80° C. under nitrogen atmosphere. The mixture was allowed to cool down to room temperature. The resulting mixture was extracted with ethyl acetate (3×50 mL). The combined organic layers were washed with NaCl (3×50 mL), dried over anhydrous Na₂SO₄. After filtration, the filtrate was concentrated under reduced pressure. The residue was purified by silica gel column chromatography, eluted with EA/PE to afford ethyl 2-{4-[(2-methyl-5-nitrophenyl)amino]-2-nitrophenyl}acetate 80 (2.4 g, 66.35%) as a yellow oil. LCMS: (M+H)⁺360.

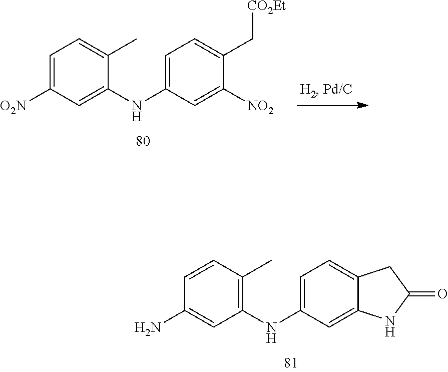 [SMILES: CCOC(=O)Cc1ccc(Nc2cc([N+](=O)[O-])ccc2C)cc1[N+](=O)[O-] ; Cc1ccc(N)cc1Nc1ccc2c(c1)NC(=O)C2]

A solution/mixture of ethyl 2-{4-[(2-methyl-5-nitrophenyl)amino]-2-nitrophenyl}acetate 80 (2.4 g, 5.566 mmol, 1 equiv) and Pd/C (598.23 mg, 5.622 mmol, 1.01 equiv) in AcOH (40 mL), THF (20 mL) was stirred for overnight at room temperature under hydrogen atmosphere. The resulting mixture was filtered, the filter cake was washed with THF (3×30 mL). The resulting mixture was concentrated under reduced pressure. The residue was purified by reversed-phase flash chromatography with the following conditions: column, C18 silica gel; mobile phase, ACN in water, 10% to 95% gradient in 30 min; detector, UV 254 nm. To obtain 6-[(5-amino-2-methylphenyl)amino]-1,3-dihydroindol-2-one 81 (1 g, 70.93%) as a yellow oil. LCMS: (M+H)⁺254. ¹H NMR (400 MHZ, Chloroform-d) δ 7.69 (s, 1H), 7.06 (d, J=7.9 Hz, 1H), 6.98 (d, J=8.0 Hz, 1H), 6.57 (dd, J=6.2, 2.2 Hz, 2H), 6.50 (d, J=2.1 Hz, 1H), 6.33 (dd, J=8.0, 2.4 Hz, 1H), 5.33 (s, 1H), 3.47 (s, 2H), 2.13 (s, 3H), 2.01 (d, J=1.8 Hz, 2H).

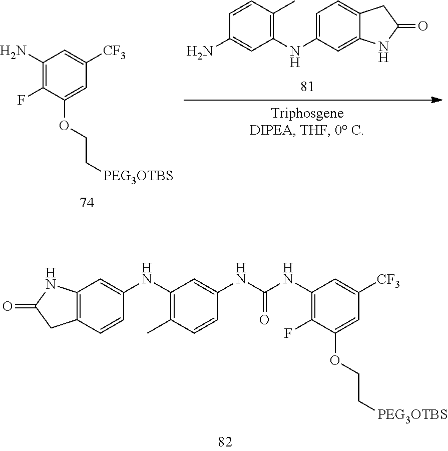

A solution of 2-fluoro-3-[(2,2,3,3-tetramethyl-4,7,10,13-tetraoxa-3-silapentadecan-15-yl)oxy]-5-(trifluoromethyl) aniline 74 (720 mg, 1.483 mmol, 1.00 equiv), triphosgene (175.99 mg, 0.593 mmol, 0.4 equiv) in THF (4 mL, 49.371 mmol, 239.74 equiv) was treated with DIEA (574.91 mg, 4.449 mmol, 3 equiv) at 0° C. under nitrogen atmosphere. The resulting mixture was stirred for 15 min at 0° C. under nitrogen atmosphere, followed by the addition of 6-[(5-amino-2-methylphenyl)amino]-1,3-dihydroindol-2-one 81 (751.16 mg, 2.966 mmol, 2.00 equiv in THF) dropwise/in portions at 0° C. The resulting mixture was stirred for 30 min at 0° C. under nitrogen atmosphere. The reaction was quenched with NaHCO₃ at 0° C. The resulting mixture was extracted with EA (3×50 mL). The combined organic layers were washed with NaCl (3×50 mL), dried over anhydrous Na₂SO₄. After filtration, the filtrate was concentrated under reduced pressure. The residue was purified by silica gel column chromatography, eluted with EA/PE (70%) to afford 1-{2-fluoro-3-[(2,2,3,3-tetramethyl-4,7,10,13-tetraoxa-3-silapentadecan-15-yl)oxy]-5-(trifluoromethyl)phenyl}-3-{4-methyl-3-[(2-oxo-1,3-dihydroindol-6-yl)amino]phenyl}urea 82 (700 mg, 61.72%) as a yellow oil. LCMS: (M+H)⁺765.

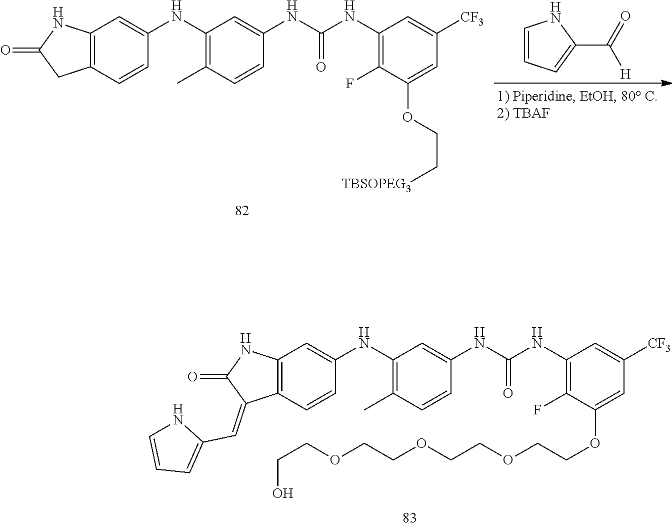

A solution/mixture of 1-{2-fluoro-3-[(2,2,3,3-tetramethyl-4,7,10,13-tetraoxa-3-silapentadecan-15-yl)oxy]-5-(trifluoromethyl)phenyl}-3-{4-methyl-3-[(2-oxo-1,3-dihydroindol-6-yl)amino]phenyl}urea 82 (700 mg, 0.915 mmol, 1 equiv) and pyrrole-2-carboxaldehyde (104.44 mg, 1.098 mmol, 1.2 equiv), piperidine (155.85 mg, 1.830 mmol, 2.0 equiv) in EtOH (7 mL) was stirred for 2 hr at 80° C. under nitrogen atmosphere. The resulting mixture was concentrated under reduced pressure. The resulting residue was diluted with THF (7 mL) and added TBAF (358.92 mg, 1.373 mmol, 1.5 equiv). The resulting mixture was stirred for 2 hr at room temperature under nitrogen atmosphere. Reaction mixture was extracted with ethyl acetate (3×20 mL). The combined organic layers were washed with NaCl (3×20 mL), dried over anhydrous Na₂SO₄. After filtration, the filtrate was concentrated under reduced pressure. The residue was purified by reversed-phase flash chromatography with the following conditions: column, C18 silica gel; mobile phase, ACN in H₂O, 10% to 95% gradient in 30 min; detector, UV 254 nm to obtain 1-[2-fluoro-3-(2-{2-[2-(2-hydroxyethoxy)ethoxy]ethoxy}ethoxy)-5-(trifluoromethyl)phenyl]-3-(4-methyl-3-{[(3Z)-2-oxo-3-(1H-pyrrol-2-ylmethylidene)-1H-indol-6-1]amino}phenyl) urea 83 (545.4 mg, 80.26%) as a red solid. LCMS: (M+H)⁺728. ¹H NMR-PH-ADAR-041-0: ¹H NMR (300 MHz, DMSO-d₆) δ 13.15 (s, 1H), 10.70 (s, 1H), 9.09 (s, 1H), 8.72 (d, J=3.0 Hz, 1H), 8.19 (dd, J=6.3, 1.9 Hz, 1H), 7.60 (s, 1H), 7.48-7.39 (m, 2H), 7.35 (d, J=2.1 Hz, 1H), 7.25 (s, 1H), 7.14 (dd, J=8.2, 5.6 Hz, 2H), 7.04 (dd, J=8.1, 2.1 Hz, 1H), 6.73-6.66 (m, 1H), 6.57 (dd, J=8.4, 2.0 Hz, 1H), 6.47 (d, J=2.0 Hz, 1H), 6.30 (q, J=2.7 Hz, 1H), 4.56 (t, J=5.4 Hz, 1H), 4.28 (t, J=4.4 Hz, 2H), 3.77 (dd, J=5.5, 3.4 Hz, 2H), 3.60 (dd, J=5.8, 3.1 Hz, 2H), 3.57-3.50 (m, 2H), 3.55-3.43 (m, 2H), 3.43-3.35 (m, 2H), 3.33 (s, 4H), 2.15 (s, 3H).

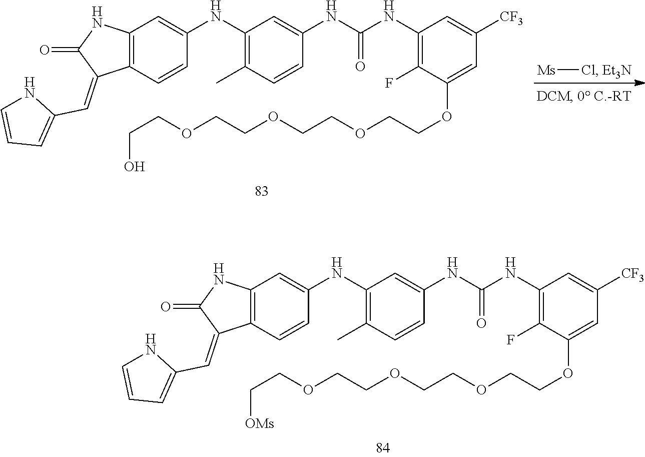

To a mixture of alcohol 83 (200 mg, 0.275 mmol, 1 eq) and triethylamine (0.1 ml, 0.7 mmol, 2.6 eq) in dry DCM (2 mL) was added methanesulfonyl chloride (0.025 mL, 0.33 mmol, 1.2 eq). The solution was stirred for 30 min at RT. LCMS showed formation of desired product with about 20% di-mesyl. Reaction mixture was quenched with aq. Saturated NaHCO₃5 mL, extracted with DCM 2×50 mL, concentrated, and purified by column chromatography using 0-20% MeOH/DCM, pure fractions were combined and concentrated to obtain the mesyl product as a brown gum (170 mg). LCMS (m/z=806 M+1) and NMR are corresponding with product. ¹H NMR (499 MHZ, DMSO-d₆) δ 13.15 (s, 1H), 10.70 (s, 1H), 9.09 (d, J=4.0 Hz, 1H), 8.71 (d, J=2.9 Hz, 1H), 8.18 (dd, J=6.2, 2.2 Hz, 1H), 7.59 (s, 1H), 7.43 (d, J=8.5 Hz, 2H), 7.38-7.33 (m, 1H), 7.25 (td, J=2.6, 1.5 Hz, 1H), 7.13 (dd, J=9.8, 6.9 Hz, 2H), 7.01 (ddd, J=25.8, 8.2, 2.2 Hz, 1H), 6.77-6.67 (m, 1H), 6.57 (dd, J=8.3, 2.0 Hz, 1H), 6.47 (d, J=2.0 Hz, 1H), 6.29 (dt, J=3.6, 2.4 Hz, 1H), 4.32-4.25 (m, 4H), 3.80-3.74 (m, 2H), 3.68-3.63 (m, 2H), 3.62-3.54 (m, 5H), 3.54 (dd, J=5.1, 2.0 Hz, 4H), 3.16 (s, 3H), 2.15 (d, J=2.7 Hz, 3H).

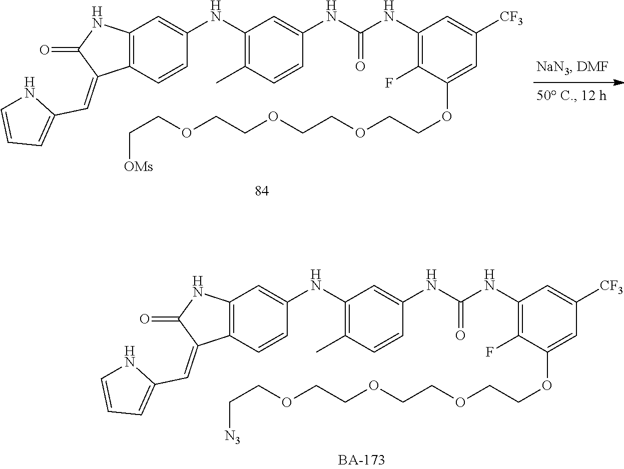 [SMILES: Cc1ccc(NC(=O)Nc2cc(C(F)(F)F)cc(OCCOCCOCCOCCOS(C)(=O)=O)c2F)cc1Nc1ccc2c(c1)NC(=O)/C2=C\c1ccc[nH]1 ; Cc1ccc(NC(=O)Nc2cc(C(F)(F)F)cc(OCCOCCOCCOCCN=[N+]=[N-])c2F)cc1Nc1ccc2c(c1)NC(=O)/C2=C\c1ccc[nH]1]

Mesyl derivative 84 (170 mg, 0.211 mmol, 1 eq), and Sodium azide (41 mg, 0.634 mmol, 3 eq) in dry DMF (1 mL) was stirred for 12 h at 50° C., Reaction mixture was cooled and diluted with water 5 mL, extracted with DCM 20 mL, dried over Na₂SO₄ and evaporated, resulting crude was purified by column chromatography using 0-20% MeOH/DCM, pure fractions were combined and concentrated to obtain azide BA-173 (60 mg, 34%) as a brick red solid (98% HPLC purity). LCMS m/z−775 (M⁺+Na) and NMR are corresponding with product. ¹H NMR (499 MHz, DMSO-d₆) δ 13.15 (s, 1H), 10.70 (s, 1H), 9.09 (s, 1H), 8.72 (d, J=2.8 Hz, 1H), 8.18 (dd, J=6.3, 2.1 Hz, 1H), 7.59 (s, 1H), 7.43 (d, J=8.4 Hz, 2H), 7.35 (d, J=2.2 Hz, 1H), 7.25 (td, J=2.6, 1.4 Hz, 1H), 7.13 (dd, J=10.7, 7.9 Hz, 2H), 7.04 (dd, J=8.2, 2.2 Hz, 1H), 6.70 (dt, J=3.7, 1.7 Hz, 1H), 6.57 (dd, J=8.3, 2.0 Hz, 1H), 6.47 (d, J=2.0 Hz, 1H), 6.29 (dt, J=3.6, 2.4 Hz, 1H), 4.30-4.25 (m, 2H), 3.80-3.74 (m, 2H), 3.62-3.57 (m, 4H), 3.57-3.49 (m, 6H), 3.37 (dd, J=5.6, 4.3 Hz, 3H), 2.15 (s, 3H).

BA-173 was conjugated to an oligo sense strand according to general procedure Type II. MW calculated: 8464.57. MW Found: 8462.57.

### Example 16: BA-179 Conjugate

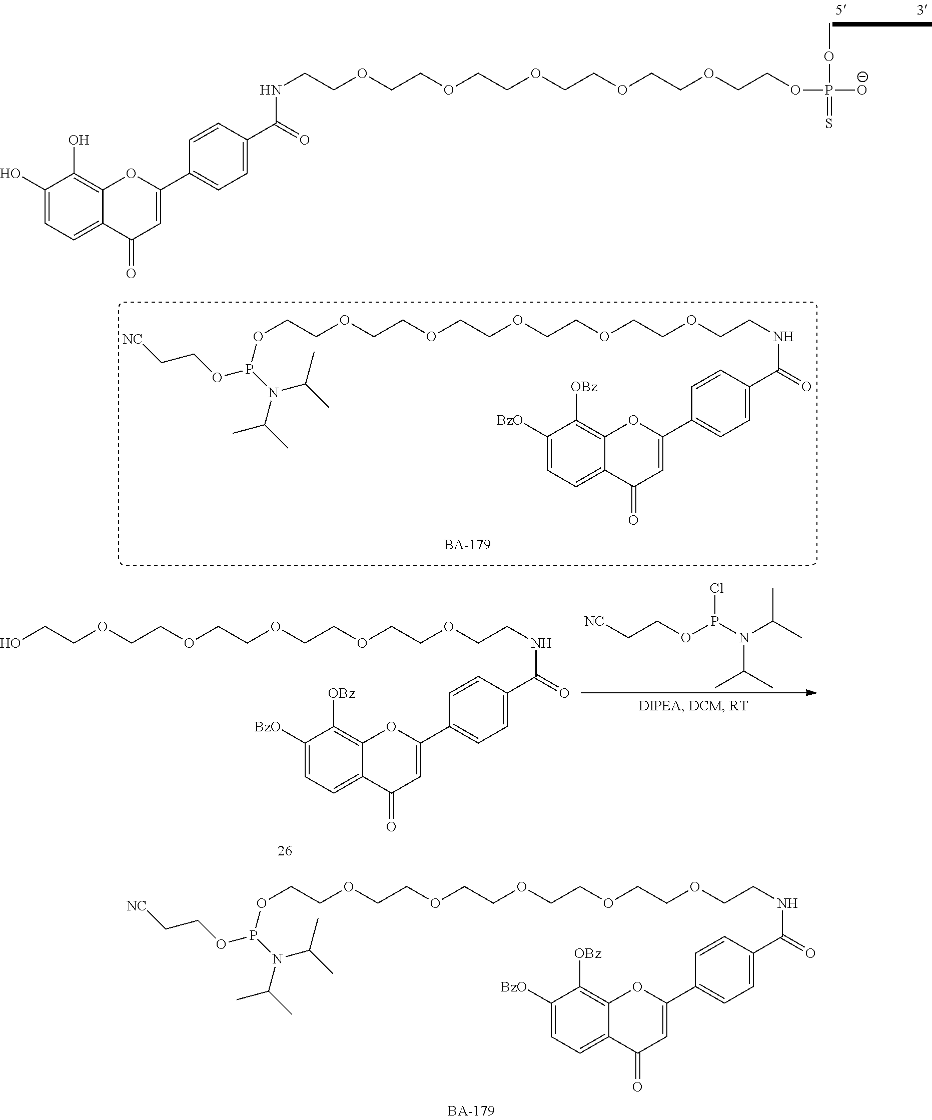

To a stirred solution Flavone alcohol (580 mg, 0.754 mmol, 1 eq) and diisopropylethylamine (0.52 mL, 3 mmol, 4 eq), in DCM (10 mL), was added N, N-diisopropyl chlorophosphoramidite (0.33 mL 1.5 mmol, 2 eq) dropwise. The reaction mixture was stirred at room temperature for 5 h. Reaction mixture was quenched with aq. saturated NaHCO₃ solution (50 mL), extracted with DCM (100) ml, washed with brine (100 mL), dried over a suitable drying agent (e.g., MgSO₄ or Na₂SO₄), and evaporated the crude product was loaded on to (pre-equilibrated with 1% Et₃N-EtOAc) Biotage silica gel column (25 g 20 μm), and purified by flash chromatography using MeOH/EtOAc 0-10% 10CV, 10% 10CV, containing 1% Et₃N as an additive, obtained 260 mg, 35% product as a pale yellow gum. 94% purity by LCMS, HPLC and P31, Mass (m/z 992 (M⁺+Na) ¹H-NMR and ³¹P-NMR are corresponding with product. ¹H NMR (500 MHZ, DMSO-d₆) δ 8.64 (t, J=5.6 Hz, 1H), 8.18-8.11 (m, 3H), 8.09-8.04 (m, 2H), 7.93 (d, J=8.6 Hz, 2H), 7.85 (d, J=8.6 Hz, 2H), 7.76-7.70 (m, 2H), 7.65-7.59 (m, 2H), 7.59-7.52 (m, 2H), 7.26 (s, 1H), 3.77-3.67 (m, 3H), 3.67-3.43 (m, 23H), 3.40 (t, J=5.7 Hz, 2H), 2.75 (td, J=5.8, 1.5 Hz, 2H), 1.11 (dd, J=6.7, 5.6 Hz, 12H). ³¹P NMR (202 MHz, DMSO-d₆) δ 147.79, 31.21, 14.28.

Subsequently, BA-179 was conjugated to an oligo sense strand. A 0.1 M solution of BA-179 was combined with 0.25M BTT activator and coupled twice for a period of ˜30 min each, sulfurized with a 0.05M DDTT solution and deprotected with 50% NH₄OH solution for 3 hours at 65° C.

### Example 17: BA-183 Conjugate

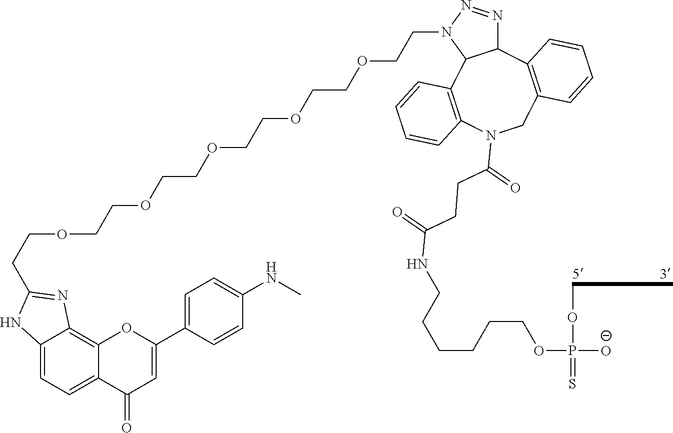 [SMILES: CNc1ccc(-c2cc(=O)c3ccc4[nH]c(CCOCCOCCOCCOCCOCCN5N=NC6c7ccccc7CN(C(=O)CCC(=O)NCCCCCCOP([O-])(=S)OC)c7ccccc7C65)nc4c3o2)cc1]

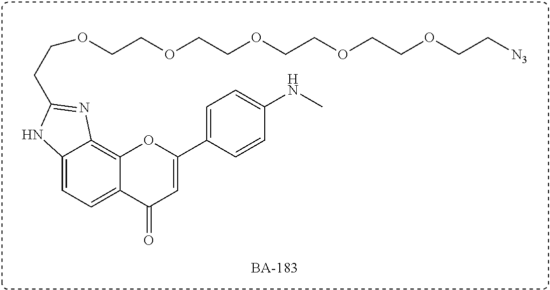 [SMILES: CNc1ccc(-c2cc(=O)c3ccc4[nH]c(CCOCCOCCOCCOCCOCCN=[N+]=[N-])nc4c3o2)cc1]

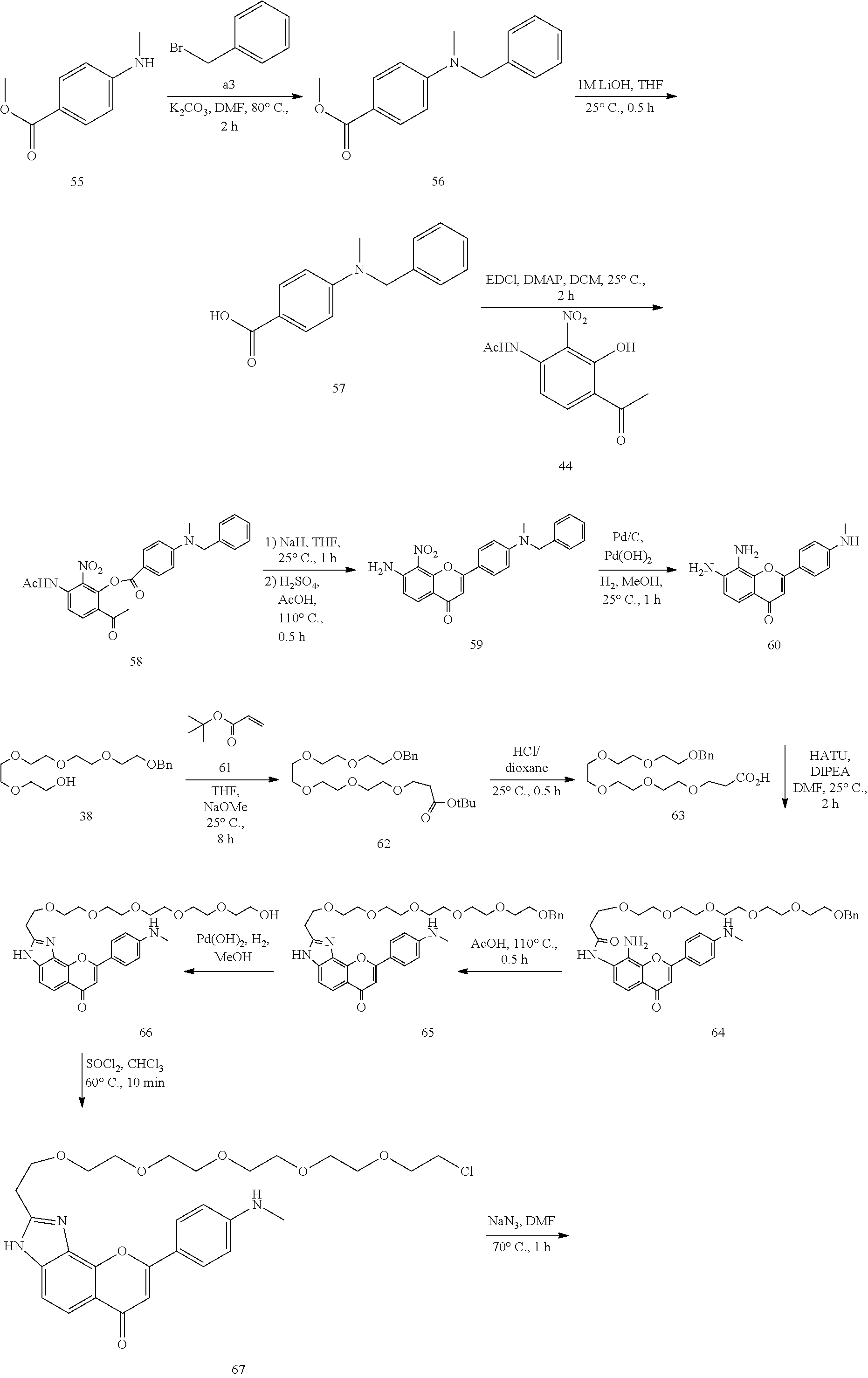

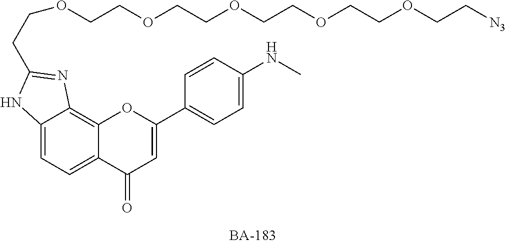 [SMILES: CNc1ccc(-c2cc(=O)c3ccc4[nH]c(CCOCCOCCOCCOCCOCCN=[N+]=[N-])nc4c3o2)cc1]

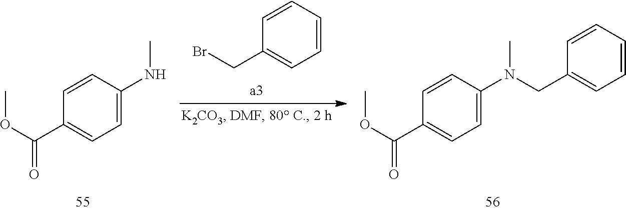 [SMILES: CNc1ccc(C(=O)OC)cc1 ; BrCc1ccccc1 ; COC(=O)c1ccc(N(C)Cc2ccccc2)cc1]

To a solution of methyl 4-(methylamino)benzoate 55 (10 g, 60.54 mmol, 1 eq), bromomethylbenzene (12.42 g, 72.64 mmol, 8.63 mL, 1.2 eq), K₂CO₃ (25.10 g, 181.61 mmol, 3 eq) was added DMF (100 mL). The mixture was stirred at 80° C. for 2 hours. LCMS showed the formation of desired product. The mixture was concentrated to afford the crude. The reaction mixture was diluted with H₂O (600 ml) and extracted with DCM (800 mL×3). The combined organic layers were dried over Na₂SO₄, filtered, and concentrated under reduced pressure to give a residue. The residue was purified by flash silica gel chromatography (ISCO®; 120 g SepaFlash® Silica Flash Column), using 0˜10% Ethyl acetate/Petroleum ether gradient @ 80 mL/min) to give the benzyl amine 56 (14.3 g, 56.01 mmol, 92.52% yield) as a yellow solid. MS ES⁺: 256.0.

¹H NMR (400 MHZ, DMSO-d₆) δ=7.77-7.70 (m, 2H), 7.35-7.29 (m, 2H), 7.27-7.16 (m, 3H), 6.75 (d, J=9.1 Hz, 2H), 4.68 (s, 2H), 3.74 (s, 3H), 3.11 (s, 3H).

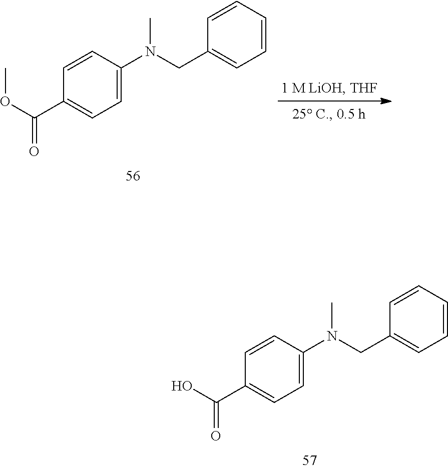 [SMILES: COC(=O)c1ccc(N(C)Cc2ccccc2)cc1 ; CN(Cc1ccccc1)c1ccc(C(=O)O)cc1]

To a solution of methyl 4-[benzyl(methyl)amino]benzoate 56 (14.3 g, 56.01 mmol, 1 eq) in THF (100 mL) was added LiOH·H₂O (1 M in H₂O, 560.10 mL, 10 eq). The mixture was stirred at 25° C. for 0.5 hr. LCMS showed the formation of desired product. The mixture was adjusted pH with 1M HCl until precipitate out white solid at 0° C. The mixture was diluted with water 200 mL and filtered, then the filter cake was dried under reduced pressure to give the acid 57 (13.49 g, 55.91 mmol, 99.82% yield) as a white solid which was used for next step without further purification. MS ES⁺: 241.9. ¹H NMR (400 MHZ, DMSO-d₆) δ=7.73 (d, J=8.8 Hz, 2H), 7.37-7.28 (m, 2H), 7.28-7.12 (m, 3H), 6.73 (d, J=8.9 Hz, 2H), 4.67 (s, 2H), 3.10 (s, 3H).

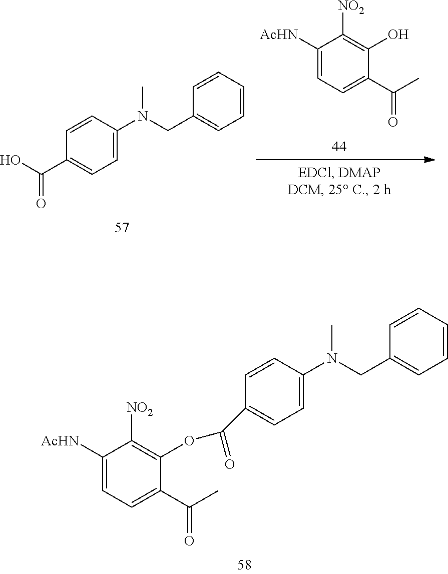 [SMILES: CN(Cc1ccccc1)c1ccc(C(=O)O)cc1 ; CC(=O)Nc1ccc(C(C)=O)c(O)c1[N+](=O)[O-] ; CC(=O)Nc1ccc(C(C)=O)c(OC(=O)c2ccc(N(C)Cc3ccccc3)cc2)c1[N+](=O)[O-]]

To a mixture of 4-[benzyl(methyl)amino]benzoic acid 57 (1.5 g, 6.22 mmol, 1 eq) and N-(4-acetyl-3-hydroxy-2-nitro-phenyl) acetamide 44 (1.48 g, 6.22 mmol, 1 eq) in DCM (15 mL) was added EDCI (2.38 g, 12.43 mmol, 2 eq) and DMAP (151.90 mg, 1.24 mmol, 0.2 eq) in one portion at 25° C. The mixture was stirred at 25° C. for 2 hr. LCMS showed the formation of desired product. The reaction mixture was concentrated under reduced pressure to give a residue. The residue was purified by flash silica gel chromatography (ISCO®; 20 g SepaFlash® Silica Flash Column, Eluent of 0˜35% Ethyl acetate/Petroleum ether gradient @ 50 mL/min) to give ester 58 (1.2 g, 2.60 mmol, 41.83% yield) as a yellow solid. MS ES⁺: 462.2.

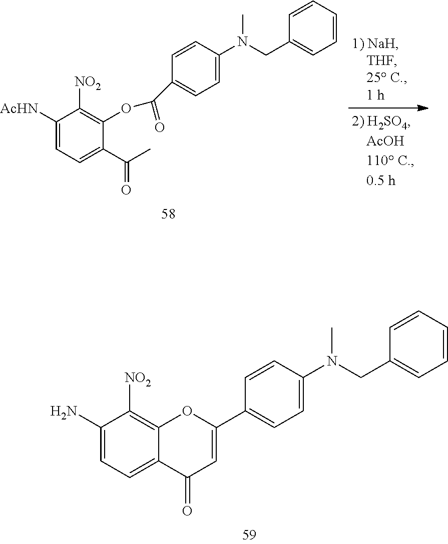 [SMILES: CC(=O)Nc1ccc(C(C)=O)c(OC(=O)c2ccc(N(C)Cc3ccccc3)cc2)c1[N+](=O)[O-] ; CN(Cc1ccccc1)c1ccc(-c2cc(=O)c3ccc(N)c([N+](=O)[O-])c3o2)cc1]

NaH (312.02 mg, 7.80 mmol, 60% purity, 3 eq) was added into (3-acetamido-6-acetyl-2-nitro-phenyl) 4-[benzyl(methyl)amino]benzoate 58 (1.2 g, 2.60 mmol, 1 eq) in THF (15 mL) one portion at 0° C. under N₂. The mixture was stirred at 0-25° C. for 1 hr. LCMS showed the formation of desired product. The mixture was poured into aq. NH₄Cl (600 mL) then extracted with EA (800 mL×3), the combined organic layers were concentrated to afford the crude. The crude (1.2 g, crude) was used for next step without further purification. To a mixture of the crude (1.2 g, 2.60 mmol, 1 eq) in AcOH (12 mL) was added H₂SO₄ (0.36 mL) in one portion at 25° C. The mixture was heated to 110° C. and stirred for 0.5 hr. LCMS showed the formation of desired product. The reaction mixture was quenched by addition aq. NaHCO₃ (800 ml) to pH=7-8 at 0° C., and then extracted with ethyl acetate (400 mL×3). The combined organic layers were dried over Na₂SO₄, filtered, and concentrated under reduced pressure to give Flavone 59 (600 mg, crude) as a yellow solid which was used for next step without further purification. MS ES⁺: 402.0.

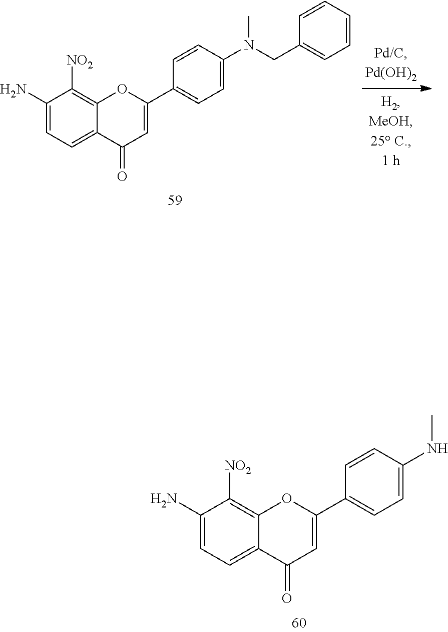 [SMILES: CN(Cc1ccccc1)c1ccc(-c2cc(=O)c3ccc(N)c([N+](=O)[O-])c3o2)cc1 ; CNc1ccc(-c2cc(=O)c3ccc(N)c([N+](=O)[O-])c3o2)cc1]

A mixture of 7-amino-2-[4-[benzyl(methyl)amino]phenyl]-8-nitro-chromen-4-one 59 (300 mg, 747.36 μmol, 1 eq), Pd/C (300 mg, 10% purity) and Pd(OH)₂ (300 mg, 20% purity) in MeOH (5 mL) was degassed and purged with H₂ for 3 times, and then the mixture was stirred at 25° C. for 1 hr under H₂ (15 Psi). LCMS showed the formation of desired product. The reaction mixture was filtered and concentrated under reduced pressure to give the diamine 60 (200 mg, 710.96 μmol, 95.13% yield) as a yellow solid which was used for next step without further purification. MS ES⁺: 282.1.

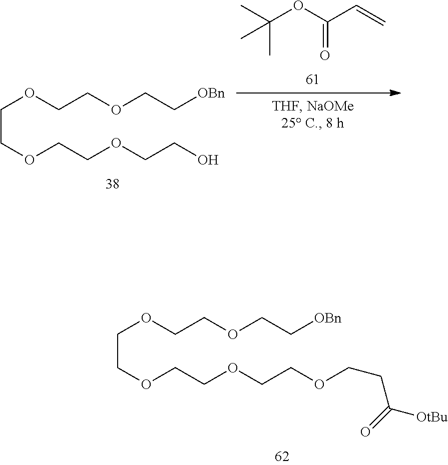

Compound tert-butyl prop-2-enoate 61 (585.42 mg, 4.57 mmol, 662.99 uL, 1.5 eq) was dissolved in THF (10 mL) under N₂ were added NaOMe (5.4 M, 5.64 uL, 0.01 eq) followed by 2-[2-[2-[2-(2-benzyloxyethoxy)ethoxy]ethoxy]ethoxy]ethanol 38 (1 g, 3.05 mmol, 1 eq). The reaction mixture was stirred at 25° C. for 8 hr. LCMS showed the formation of desired product. The reaction mixture was quenched by addition of 30 mL H₂O and partitioned between 50 mL ethyl acetate and 20 mL brine. The aqueous phase was further extracted with EA (2×40 mL) and the combined organic phases were washed with brine (2×50 mL) and dried over Na₂SO₄. The residue was purified by flash silica gel chromatography (ISCO®; 20 g SepaFlash® Silica Flash Column, Eluent of 0˜30% Ethyl acetate/Petroleum ether gradient @ 50 mL/min) to give t-butyl ester 62 (960 mg, 2.10 mmol, 69.05% yield) as a yellow oil. MS ES⁺: 456. ¹H NMR (400 MHZ, DMSO-d₆) δ=7.41-7.19 (m, 5H), 4.48 (s, 2H), 3.59-3.48 (m, 22H), 2.41 (t, J=6.3 Hz, 2H), 1.39 (s, 9H).

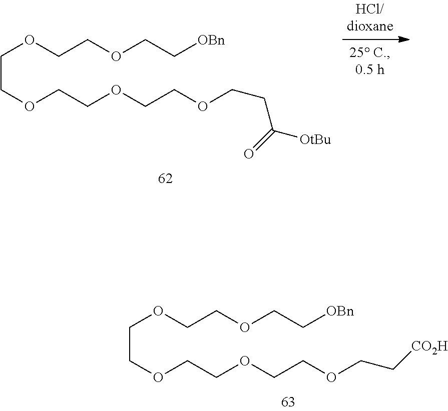 [SMILES: CC(C)(C)OC(=O)CCOCCOCCOCCOCCOCCOCc1ccccc1 ; O=C(O)CCOCCOCCOCCOCCOCCOCc1ccccc1]

To a solution of tert-butyl 3-[2-[2-[2-[2-(2-benzyloxyethoxy)ethoxy]ethoxy]ethoxy]ethoxy]propanoate 62 (640 mg, 1.40 mmol, 1 eq) in HCl/dioxane (3 mL). The mixture was stirred at 25° C. for 0.5 hr. LCMS showed the formation of desired product. The reaction mixture was concentrated under reduced pressure to give acid 63 (600 mg, crude) as a yellow oil which was used for next step without further purification. MS ES⁺: 401.2.

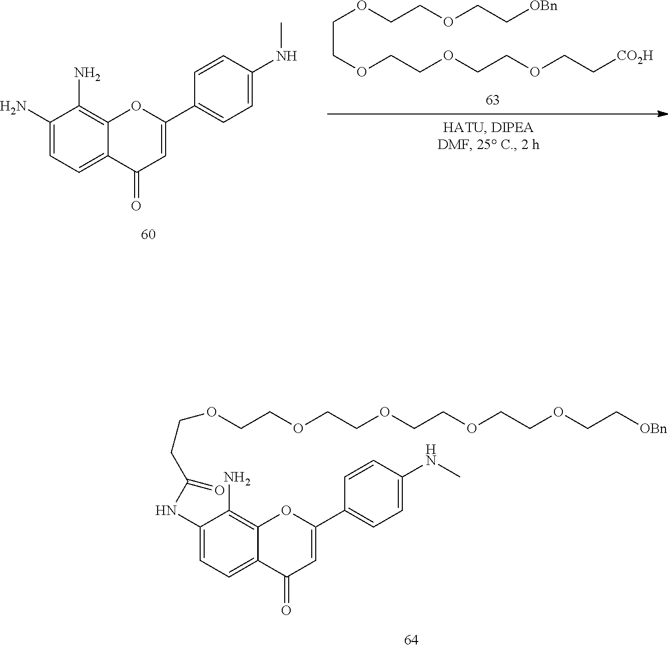 [SMILES: CNc1ccc(-c2cc(=O)c3ccc(N)c(N)c3o2)cc1 ; O=C(O)CCOCCOCCOCCOCCOCCOCc1ccccc1 ; CNc1ccc(-c2cc(=O)c3ccc(NC(=O)CCOCCOCCOCCOCCOCCOCc4ccccc4)c(N)c3o2)cc1]

To a mixture of 7,8-diamino-2-[4-(methylamino)phenyl]chromen-4-one 60 (290 mg, 1.03 mmol, 1 eq) and 3-[2-[2-[2-[2-(2-benzyloxyethoxy)ethoxy]ethoxy]ethoxy]ethoxy]propanoic acid 63 (412.83 mg, 1.03 mmol, 1 eq) in DMF (2 mL) was added DIPEA (399.71 mg, 3.09 mmol, 538.69 uL, 3 eq) and HATU (587.97 mg, 1.55 mmol, 1.5 eq) in one portion at 0° C. The mixture was stirred at 25° C. for 0.5 hr. LCMS showed the formation of desired product. The reaction mixture was concentrated under reduced pressure to give a residue. The residue was purified by flash silica gel chromatography (ISCO®; 12 g SepaFlash® Silica Flash Column, Eluent of 0˜10% Ethyl acetate/Petroleum ether gradient @ 40 mL/min) to give the amide 64 (490 mg, 738.22 μmol, 71.61% yield) as a yellow oil. MS ES⁺: 664.4.

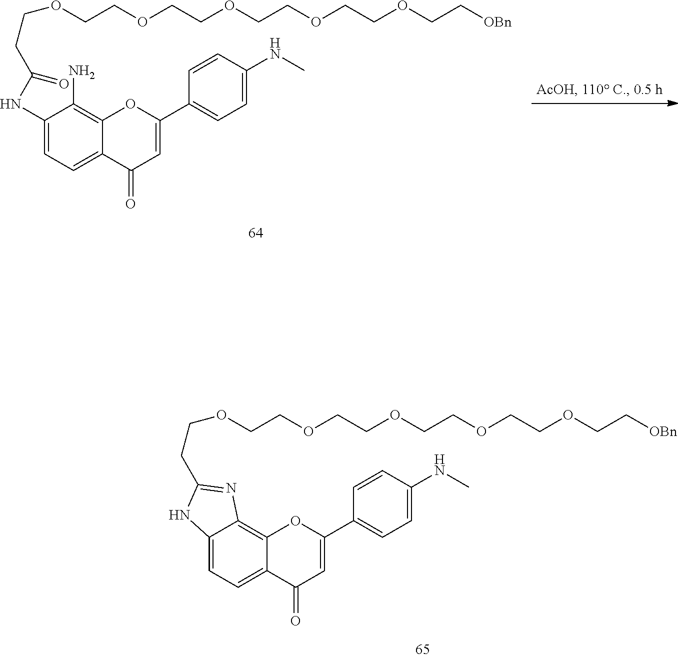 [SMILES: CNc1ccc(-c2cc(=O)c3ccc(NC(=O)CCOCCOCCOCCOCCOCCOCc4ccccc4)c(N)c3o2)cc1 ; CNc1ccc(-c2cc(=O)c3ccc4[nH]c(CCOCCOCCOCCOCCOCCOCc5ccccc5)nc4c3o2)cc1]

N-[8-amino-2-[4-(methylamino)phenyl]-4-oxo-chromen-7-yl]-3-[2-[2-[2-[2-(2-benzyloxyethoxy)ethoxy]ethoxy]ethoxy]ethoxy]propanamide 64 (490 mg, 738.22 μmol, 1 eq) in AcOH (10 mL) was heated to 110° C. and stirred for 0.5 hr. LCMS showed the formation of desired product. The reaction mixture was quenched by addition aq. NaHCO₃ (300 ml) to pH=7-8 at 0° C., and then extracted with EA (200 mL×3). The combined organic layers were dried over Na₂SO₄, filtered, and concentrated under reduced pressure to give the imidazole 65 (300 mg, crude) as a yellow oil which was used for next step without further purification. MS ES⁺: 646.2.

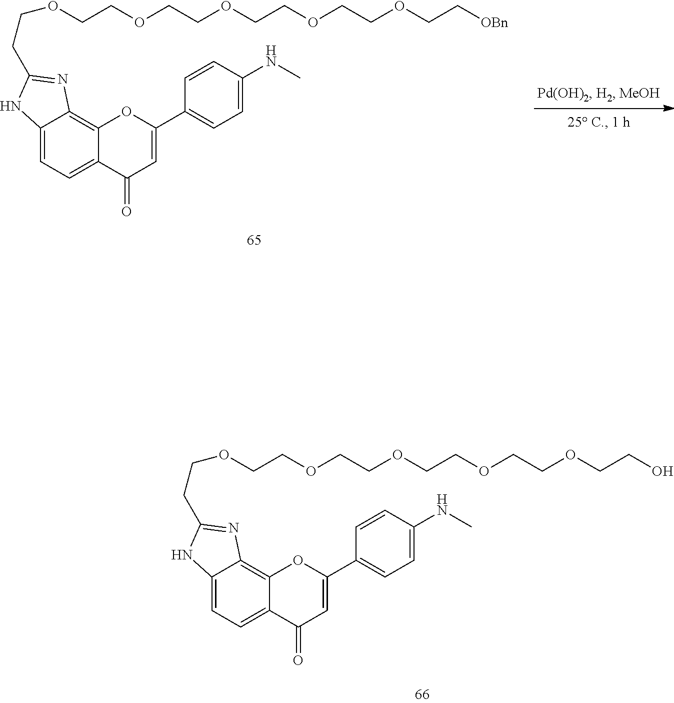 [SMILES: CNc1ccc(-c2cc(=O)c3ccc4[nH]c(CCOCCOCCOCCOCCOCCOCc5ccccc5)nc4c3o2)cc1 ; CNc1ccc(-c2cc(=O)c3ccc4[nH]c(CCOCCOCCOCCOCCOCCO)nc4c3o2)cc1]

A mixture of 2-[2-[2-[2-[2-[2-(2-benzyloxyethoxy)ethoxy]ethoxy]ethoxy]ethoxy]ethyl]-8-[4-(methylamino)phenyl]-3H-pyrano[2,3-e]benzimidazol-6-one 65 (300 mg, 464.58 umol, 1 eq), Pd(OH)₂ (255.32 mg, 363.61 umol, 20% purity) in MeOH (10 mL) was degassed and purged with H₂ gas for 3 times, and then the mixture was stirred at 25° C. for 1 hr under H₂ (15 Psi). LCMS showed the formation of desired product. The reaction mixture was filtered and concentrated under reduced pressure to give a residue. The residue was purified by flash silica gel chromatography (ISCO®; 12 g SepaFlash® Silica Flash Column, Eluent of 0˜10% Ethyl acetate/Petroleum ether gradient @ 40 mL/min) to give the alcohol 66 (185 mg, 332.96 umol, 71.67% yield) as a yellow oil. MS ES⁺: 556.2.

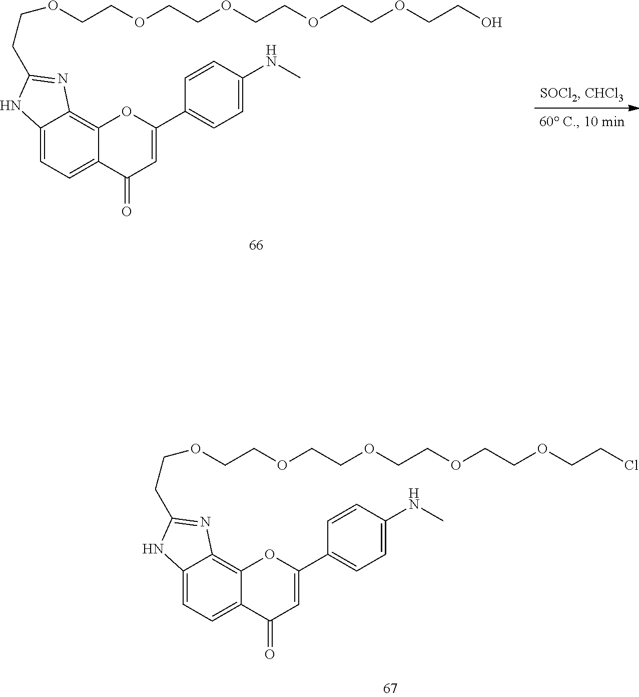 [SMILES: CNc1ccc(-c2cc(=O)c3ccc4[nH]c(CCOCCOCCOCCOCCOCCO)nc4c3o2)cc1 ; CNc1ccc(-c2cc(=O)c3ccc4[nH]c(CCOCCOCCOCCOCCOCCCl)nc4c3o2)cc1]

To a solution of 2-[2-[2-[2-[2-[2-(2-hydroxyethoxy)ethoxy]ethoxy]ethoxy]ethoxy]ethyl]-8-[4-(methylamino)phenyl]-3H-pyrano[2,3-e]benzimidazol-6-one 66 (240 mg, 431.95 umol, 1 eq) in CHCl₃ (0.5 mL) was added SOCl₂ (513.89 mg, 4.32 mmol, 313.35 μL, 10 eq) dropwise at 0° C. The mixture was stirred at 60° C. for 10 min. LCMS showed the formation of desired product. The reaction mixture was quenched by addition aq. NaHCO₃ (5 ml) to pH=7-8 at 0° C., and then extracted with EA (30 mL×3). The combined organic layers were dried over Na₂SO₄, filtered, and concentrated under reduced pressure to give a residue. The crude product was purified by prep. HPLC (Welch Xtimate C18 150×30 mm×5 um); Mobile Phase A [water (NH₃H₂O+NH₄HCO₃)-ACN]; Mobile Phase B: acetonitrile, Flow rate: 25 mL/min, gradient condition from 14% B to 54%). The pure fractions were collected, and the volatiles were removed under vacuum. The residue was partitioned between acetonitrile (2 mL) and water (10 mL). The solution was lyophilized to dryness to give the chloride 67 (25.02 mg, 41.62 μmol, 9.64% yield, 95.5% purity) as a yellow oil. MS ES⁺: 574.5, ¹H NMR (400 MHZ, DMSO-d₆) δ=8.02-7.86 (m, 2H), 7.77 (d, J=8.5 Hz, 1H), 7.52 (d, J=8.4 Hz, 1H), 6.76 (s, 1H), 6.70 (d, J=8.9 Hz, 2H), 6.55 (q, J=4.6 Hz, 1H), 3.91 (t, J=6.6 Hz, 2H), 3.71-3.61 (m, 4H), 3.59-3.55 (m, 2H), 3.52 (dd, J=2.9, 5.4 Hz, 4H), 3.48-3.43 (m, 10H), 3.17 (br t, J=6.6 Hz, 2H), 2.78 (d, J=4.9 Hz, 3H).

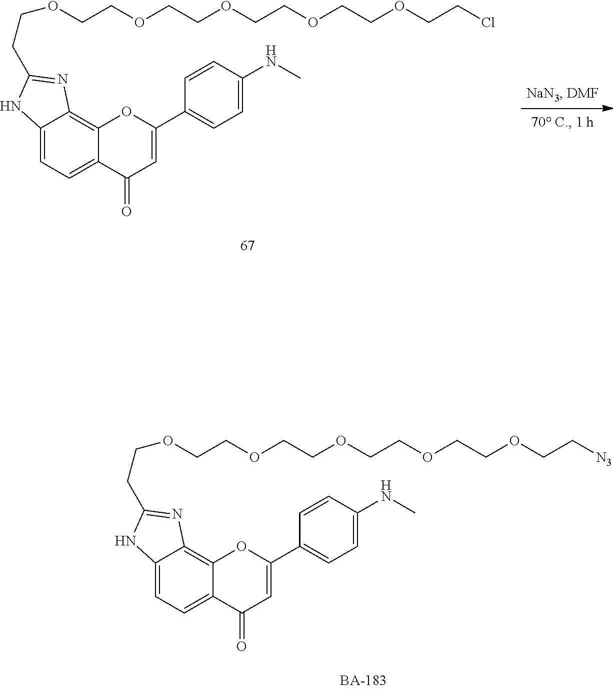 [SMILES: CNc1ccc(-c2cc(=O)c3ccc4[nH]c(CCOCCOCCOCCOCCOCCCl)nc4c3o2)cc1 ; CNc1ccc(-c2cc(=O)c3ccc4[nH]c(CCOCCOCCOCCOCCOCCN=[N+]=[N-])nc4c3o2)cc1]

PEG-5-Chloro Flavone 67 (57 mg, 0.099 mmol, 1 eq), and sodium azide (32 mg, 0.497 mmol, 5 eq) in dry DMF (1 mL) was stirred for 12 h at 70° C., Reaction mixture was cooled and diluted with Brine 20 mL, extracted with DCM 50 mL, dried over Na₂SO₄ and evaporated, resulting crude was purified by column chromatography using 0-10% MeOH/DCM, pure fractions were combined and concentrated to obtain azide BA-183 (29:71 tautomeric mixture)(51 mg, 88%) as a yellow gum. LCMS m/z 580, 603 (M+Na) and NMR are corresponding with product. >95% LCMS and HPLC purity. ¹H NMR (499 MHz, DMSO-d₆) δ 12.83 (s, 1H), 7.92 (s, 1H), 7.77 (d, J=8.5 Hz, 1H), 7.52 (s, 1H), 6.76 (s, 1H), 6.73-6.67 (m, 2H), 6.56 (q, J=4.9 Hz, 1H), 3.91 (t, J=6.6 Hz, 2H), 3.60-3.40 (m, 18H), 3.36 (dd, J=5.6, 4.3 Hz, 2H), 3.17 (t, J=6.6 Hz, 2H), 2.78 (d, J=4.9 Hz, 3H).

BA-183 was conjugated to an oligo sense strand according to general procedure Type II. MW calculated: 8292.05. MW Found: 8290.63.

### Example 18: BA-196, BA-197, and BA-198 Conjugates

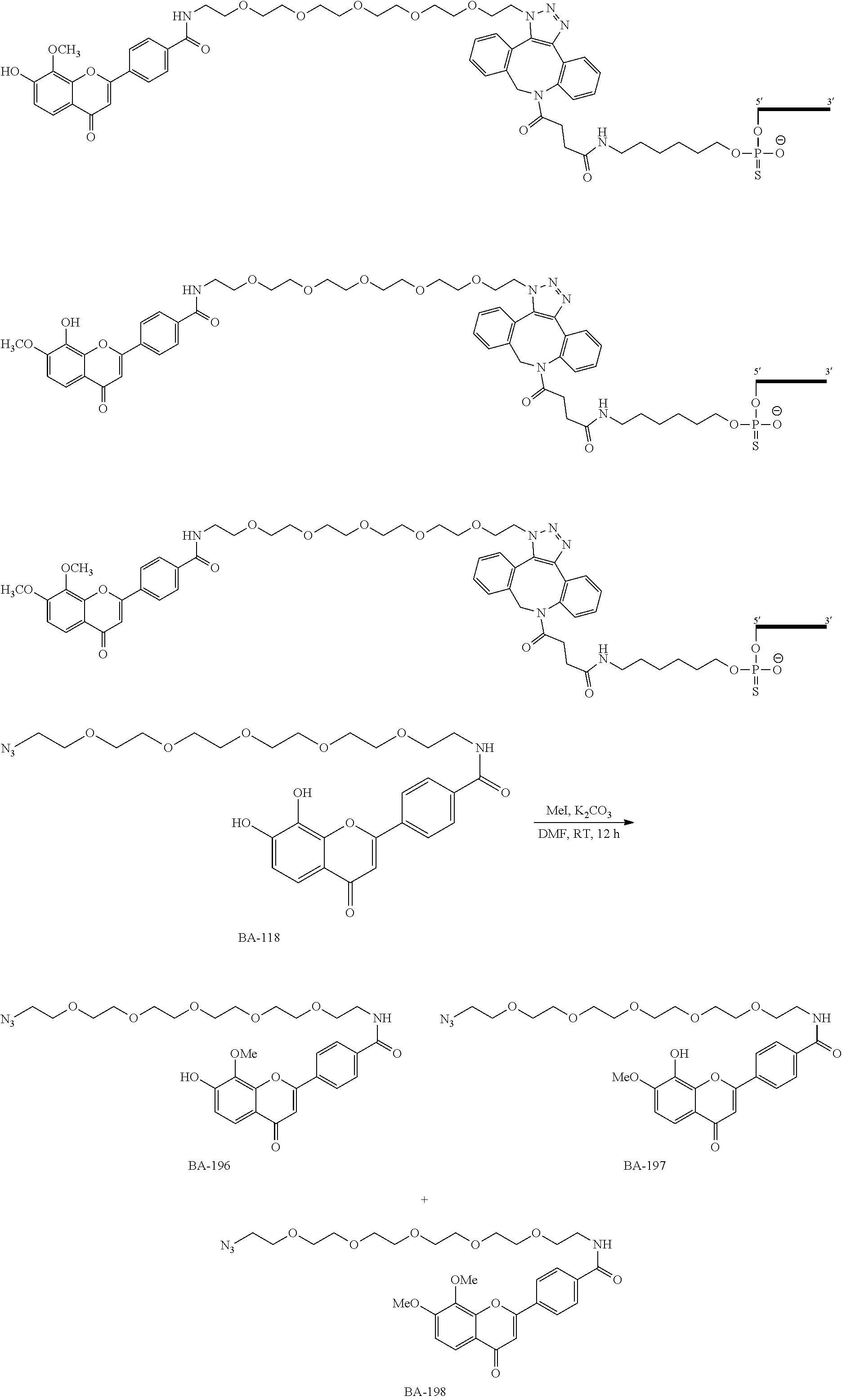

To a stirred suspension of dihydroxy flavone (400 mg, 0.683 mmol, 1 eq) and K₂CO₃ (188 mg, 1.365 mmol, 2 eq) in DMF (10 mL) was added Iodomethane (0.043 mL, 0.683 mmol, 1 eq), the mixture was stirred overnight at RT. LCMS showed mixture of mono and di-methylation and starting material. Reaction mixture was concentrated, the crude was purified by reverse phase column chromatography using 0-60% Water/MeCN with 0.1% formic acid. as eluent pure fractions were combined and concentrated to obtain desired product BA-196, 50 mg (12%, yield with 85% purity by NMR, contains 15% Regio isomer), its Regio isomer BA-197, 15 mg (4% yield, with 65% purity by NMR contains 35% Regio isomer) and di-methylated product BA-198, 100 mg (24% yield with 100% pure) as yellow sticky solids. NMR and LCMS (m/z=601 M+1) for mono and (m/z=615 M+1) for di-methoxy are corresponding with product.

BA-196 NMR: ¹H NMR (500 MHZ, DMSO-d₆) δ 10.66 (s, 1H), 8.71 (t, J=5.5 Hz, 1H), 8.16 (d, J=8.38 Hz, 2H), 8.04 (d, J=8.38 Hz, 2H) 7.65 (d, J=8.84 Hz, 1H), 7.03 (s, 1H), 7.02 (d, J=8.84 Hz, 1H), 3.96 (s, 1H), 3.60-3.42 (m, 20H), 3.47-3.42 (m, 2H), 3.40-3.34 (m, 2H).

BA-197-NMR: ¹H NMR (500 MHZ, DMSO-d₆) δ 9.75 (s, 1H), 8.71 (q, J=5.8 Hz, 1H), 8.23 (d, J=8.6 Hz, 2H), 8.02 (d, J=8.6 Hz, 2H), 7.53 (d, J=8.9 Hz, 1H), 7.22 (d, J=8.9 Hz, 1H), 7.03 (s, 1H), 3.95 (s, 3H), 3.62-3.42 (m, 24H), 3.39-3.35 (m, 2H).

BA-198-NMR: ¹H NMR (500 MHZ, DMSO-d₆) δ 8.71 (t, J=5.6 Hz, 1H), 8.16 (d, J=8.5 Hz, 2H), 8.04 (d, J=8.5 Hz, 2H), 7.80 (d, J=9.0 Hz, 1H), 7.30 (d, J=9.0 Hz, 1H), 7.07 (s, 1H), 3.97 (s, 3H), 3.96 (s, 3H), 3.60-3.42 (m, 22H), 3.40-3.34 (m, 2H).

BA-196, BA-197, and BA-198 will each be independently conjugated to an oligo sense strand according to general procedure Type II.

### Example 19: BA-141 Conjugate

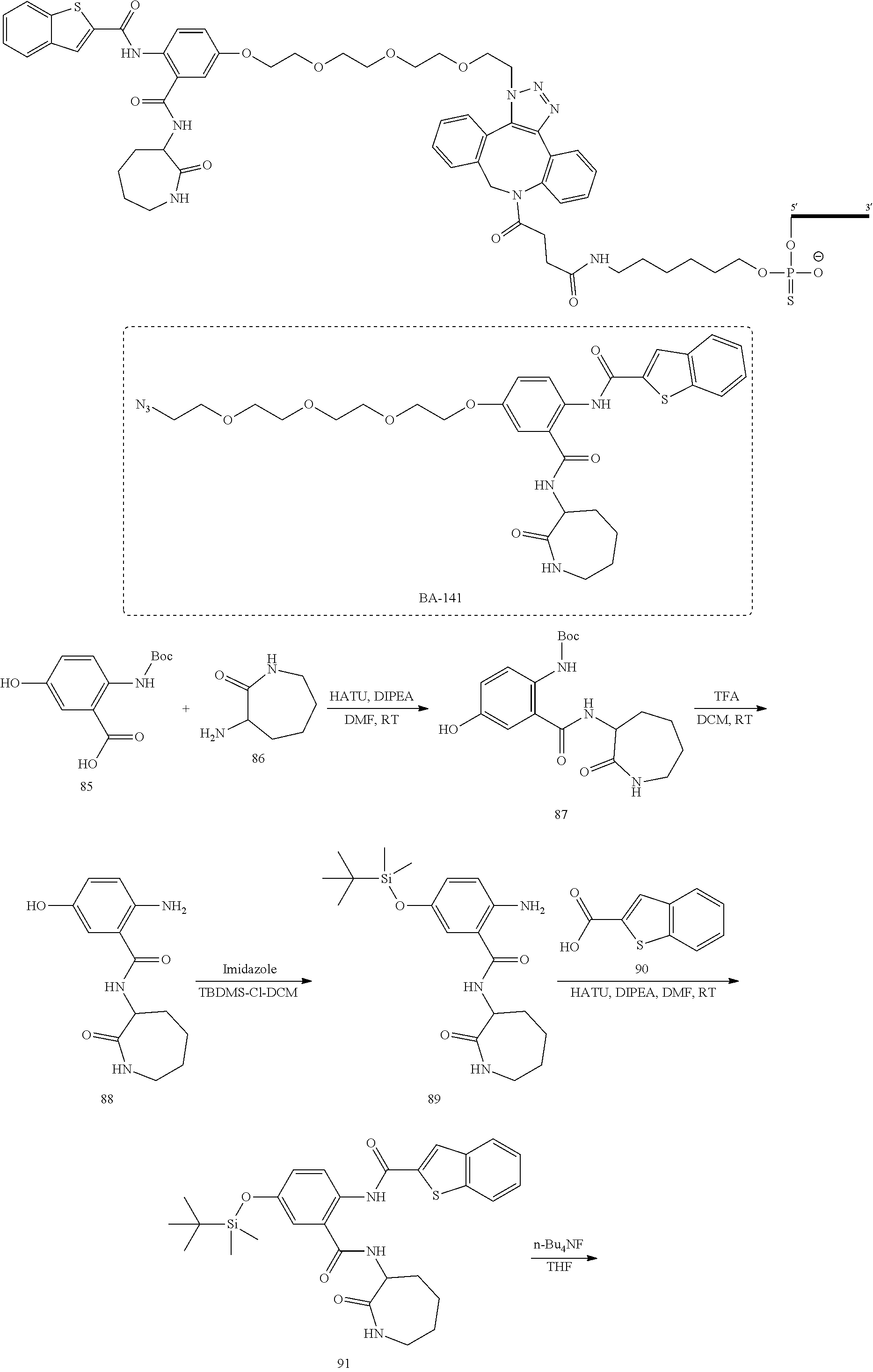

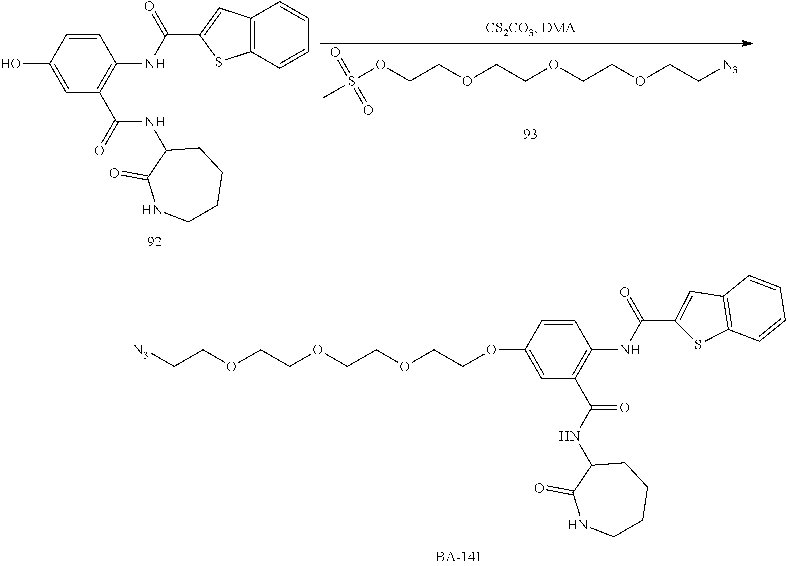

The acid 85 (1.63 g, 6.4 mmol) and amine 86 (1.0 g, 7.7 mmol, 1.2 equiv.) were dissolved in DMF (10 mL) and treated with DIPEA (1.7 mL, 9.65 mmol, 1.5equiv.) and HATU (3.67 g, 9.65 mmol, 1.5 equiv.). The reaction mixture was stirred at 20° C. for 3d. LCMS data showed the desired product. The mixture was poured into water (60 mL) and extracted with ethyl acetate (2×50 mL). The combined extracts were washed with water (3×50 mL), sat. NaHCO₃, sat. NH₄Cl, water and brine. Drying over Na₂SO₄ and concentration yielded 87 (2.60 g 100.04%) as a light-brown solid. The crude material was used as such in the next step. LCMS data: t=2.15 min, MS(+) m/z: 364.3 for [C₁₈H₂₆N₃O₅]⁺ [M+H]⁺; 386.3 for [C₁₈H₂₅NaN₃O₅]⁺ [M+Na]⁺; 749.6 for [C₃₆H₅₀NaN₆O₁₀]⁺ [2M+Na]⁺; MS(−) m/z: 362.3 for [C₁₈H₂₄N₃O₅]− [M−H]−.

¹H NMR (499 MHz, DMSO-d₆) δ 9.85 (s, 1H), 9.42 (s, 1H), 8.32 (d, J=6.8 Hz, 1H), 7.92-7.83 (m, 2H), 7.10 (d, J=2.8 Hz, 1H), 6.88 (dd, J=9.0, 2.8 Hz, 1H), 4.62-4.51 (m, 1H), 3.24 (ddd, J=15.8, 11.4, 5.0 Hz, 1H), 3.14-3.06 (m, 1H), 1.96-1.84 (m, 2H), 1.82-1.73 (m, 1H), 1.69 (tt, J=15.3, 3.9 Hz, 1H), 1.59-1.50 (m, 1H), 1.44 (s, 9H), 1.31-1.19 (m, 1H). The LCMS and NMR data is consistent with the structure of the desired product.

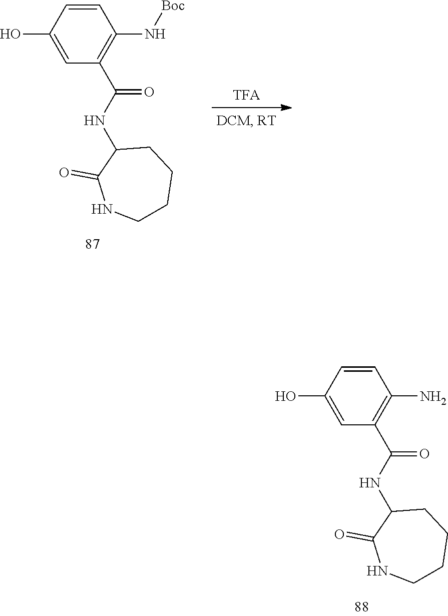 [SMILES: CC(C)(C)OC(=O)Nc1ccc(O)cc1C(=O)NC1CCCCNC1=O ; Nc1ccc(O)cc1C(=O)NC1CCCCNC1=O]

The carbamate 87 (2.62 g, 7.2 mmol) was dissolved/suspended in DCM (20 mL) and treated with TFA (7.1 mL, 108 mmol, 15 equiv.). The reaction mixture became homogenous. It was stirred at 20° C. for 2 h. LCMS data showed the presence of the desired product. The mixture was concentrated to dryness, redissolved in DCM (30 mL) and reconcentrated to yield 88 (1.84 g 97%) as a thick brown oil. LCMS data: t=0.87 min, MS(+) m/z: 264.2 for [C₁₃H₁₈N₃O₃]⁺ [M+H]⁺; 286.2 for [C₁₃H₁₇N₃NaO₃]⁺ [M+H]⁺. ¹H NMR (499 MHz, DMSO-d₆) δ 8.63 (d, J=7.2 Hz, 1H), 7.95 (dd, J=7.4, 4.9 Hz, 1H), 7.29-7.16 (m, 2H), 7.00 (dd, J=8.6, 2.7 Hz, 1H), 4.65-4.54 (m, 1H), 3.23 (ddd, J=15.7, 11.0, 4.8 Hz, 1H), 3.16-3.00 (m, 1H), 1.95-1.85 (m, 2H), 1.83-1.75 (m, 1H), 1.68 (qt, J=10.8, 2.6 Hz, 1H), 1.61-1.49 (m, 1H), 1.26 (dtd, J=14.5, 11.9, 3.2 Hz, 1H).

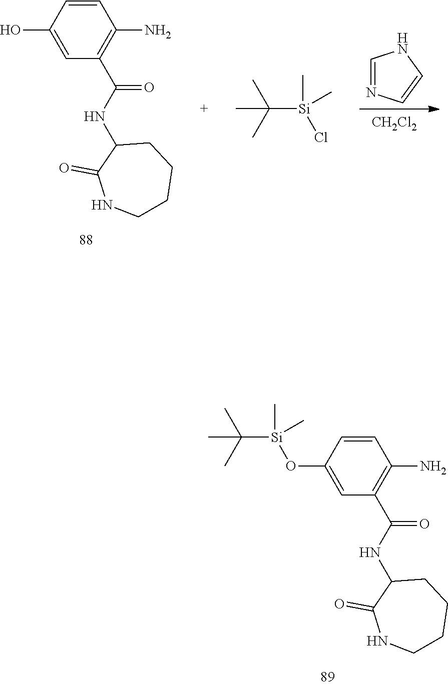

The aminophenol 88 (1.50 g, 5.7 mmol) was dissolved in DCM (25 mL) and treated with imidazole (1.2 g, 17 mmol, 3 equiv.) and TBDMS chloride (0.95 g, 6.3 mmol, 1.1 equiv.). The reaction mixture was stirred at 20° C. for 2 h. LCMS data showed the presence of the desired product. The mixture was treated with sat. NaHCO₃ (5 mL) and extracted with DCM. Washing with water, drying over Na₂SO₄ and conc. yielded 89 (1.77 g 82%) as a brown oil. LCMS data: t=0.82 min, MS(+) m/z: 378.1 for [C₁₉H₃₂N₃O₃Si]⁺ [M+H]⁺; 400.4 for [C₁₉H₃₁N₃NaO₃Si]⁺ [M+Na]⁺; 755.8 for [C₃₈H₆₃N₆O₆Si₂]⁺ [2M+H]⁺; 777.7 for [C₃₈H₆₂N₆NaO₆Si₂]⁺ [2M+Na]⁺. ¹H NMR (499 MHz, DMSO-d₆) δ 7.98 (d, J=6.8 Hz, 1H), 7.86 (dd, J=7.5, 4.9 Hz, 1H), 6.94 (d, J=2.8 Hz, 1H), 6.74 (dd, J=8.7, 2.8 Hz, 1H), 6.62 (d, J=8.7 Hz, 1H), 5.88 (s, 2H), 4.54 (ddd, J=11.2, 6.8, 1.6 Hz, 1H), 3.22 (ddd, J=15.7, 11.2, 4.8 Hz, 1H), 3.13-3.05 (m, 1H), 1.94-1.84 (m, 2H), 1.82-1.74 (m, 1H), 1.73-1.60 (m, 1H), 1.59-1.47 (m, 1H), 1.23 (dd, J=26.8, 14.2 Hz, 1H), 0.94 (s, 9H), 0.14 (s, 6H). TMS chloride is present in the final product. No purification was performed.

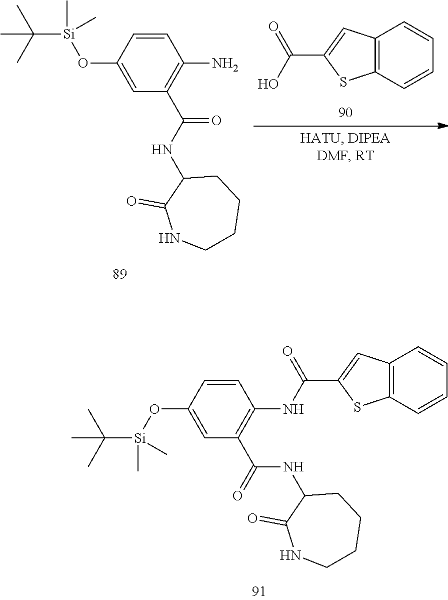 [SMILES: CC(C)(C)[Si](C)(C)Oc1ccc(N)c(C(=O)NC2CCCCNC2=O)c1 ; O=C(O)c1cc2ccccc2s1 ; CC(C)(C)[Si](C)(C)Oc1ccc(NC(=O)c2cc3ccccc3s2)c(C(=O)NC2CCCCNC2=O)c1]

The acid 90 (0.52 g, 2.9 mmol) was dissolved/suspended in dry DMF (5 mL) and treated with DIPEA (1.5 mL, 8.8 mmol, 3 equiv.) and HATU (1.66 g, 4.4 mmol, 1.5 equiv.). The reaction mixture was stirred at 20° C. for 10 min. Aniline 89 (1.3 g, 3.4 mmol, 1.2 equiv.) was added and the mixture was stirred at 20° C. for 4 h. A fine ppt has formed. LCMS data is consistent with the presence of the desired product. The mixture was poured in 200 ml of water. A beige ppt formed. Swirling and sonication yielded a suspension of a well-defined solid. The product was collected by filtration, washed with water (2×50 mL) and air-dried. LCMS data: t=2.28 min, MS(+) m/z: 538.5 for [C₂₈H₃₆N₄O₅SSi]⁺ [M+H]⁺; 560.5 for [C₂₈H₃₅N₄NaO₅SSi]⁺ [M+Na]⁺. A free-flowing beige solid 91 (1.55 g, 98%) was obtained. The NMR data is consistent with the structure of the desired product. ¹H NMR (499 MHz, DMSO-d₆) δ 11.83 (s, 1H), 8.65 (d, J=7.1 Hz, 1H), 8.25 (d, J=8.9 Hz, 1H), 8.11-8.02 (m, 3H), 7.90 (dd, J=7.5, 4.9 Hz, 1H), 7.57-7.44 (m, 2H), 7.33 (d, J=2.8 Hz, 1H), 7.10 (dd, J=8.9, 2.8 Hz, 1H), 4.65 (ddd, J=11.6, 7.2, 1.6 Hz, 1H), 3.30-3.20 (m, 1H), 3.14-3.06 (m, 2H), 1.96-1.87 (m, 3H), 1.83-1.65 (m, 1H), 1.65-1.48 (m, 1H), 1.24 (d, J=12.8 Hz, 2H), 0.98 (s, 9H), 0.24 (s, 6H).

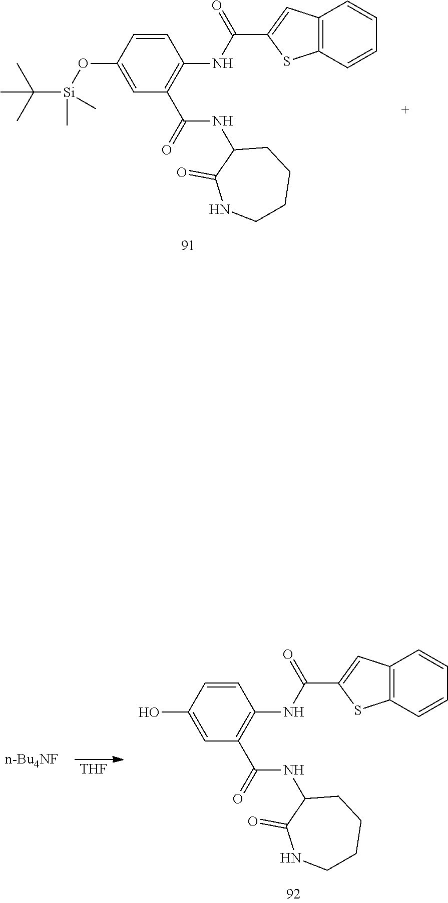 [SMILES: CC(C)(C)[Si](C)(C)Oc1ccc(NC(=O)c2cc3ccccc3s2)c(C(=O)NC2CCCCNC2=O)c1 ; "n-Bu4NF" ; O=C(Nc1ccc(O)cc1C(=O)NC1CCCCNC1=O)c1cc2ccccc2s1 ; "THF"]

The silyl ether 91 (1.5 g, 2.8 mmol) was dissolved/suspended in dry THF (3 mL) and treated with a solution of TBAF in THF (3.6 mL, 3.6 mmol, 1.3 equiv.). The mixture was stirred at 20° C. for 4 h. LCMS data is consistent with the presence of the desired product: t=2.35 min, MS(+) m/z: 424.3 for [C₂₂H₂₂N₃O₄S]⁺ [M+H]⁺; 446.2 for [C₂₂H₂₁N₃NaO₄S]⁺ [M+Na]⁺; 847.5 for [C₄₄H₄₃N₆O₈S₂]⁺ [2M+H]⁺; 869.6 for [C₄₄H₄₂N₆NaO₈S₂]⁺ [2M+Na]⁺; MS(−) m/z: 422.4 for [C₂₂H₂₀N₃O₄S]− [M−H]−. The reaction mixture was diluted with ethyl acetate (120 mL) and washed with sat. NH₄Cl and brine. Concentration yielded a brown oil. Silicagel purification (25% to 100% [3:1 ethyl acetate/ethanol] in hexanes) yielded two main fractions: Fraction 1: tubes A12-A17 (35% elution mix). Consistent with the desired product. 0.16 g. Fraction 2: tubes A18 to end (100% elution mix). Consistent with tetrabutylammonium salts. A fair amount of material was left on the column. Methanol could not elute it. The silica was recovered from the column and was treated with DMF (50 mL). The extract was filtered off, the silica was washed with more DMF and the combined extracts were poured into water (350 mL). A fine, off-white ppt was obtained. It was collected by filtration using a fine porosity. It was washed with more water and air-dried to yield phenol 92 (0.76 g, 64%) of an off-white solid. The NMR data is consistent with the structure of the desired product: ¹H NMR (499 MHz, DMSO-d₆) δ 11.63 (s, 1H), 9.70 (s, 1H), 8.47 (d, J=6.9 Hz, 1H), 8.12 (d, J=8.9 Hz, 1H), 8.08-8.03 (m, 3H), 7.97-7.89 (m, 1H), 7.49 (pd, J=7.2, 1.5 Hz, 2H), 7.22 (d, J=2.8 Hz, 1H), 6.98 (dd, J=8.9, 2.8 Hz, 1H), 4.62 (dd, J=11.2, 6.8 Hz, 1H), 3.25 (td, J=11.4, 11.0, 5.7 Hz, 1H), 3.13 (ddd, J=33.2, 12.4, 6.3 Hz, 1H), 1.92 (dd, J=12.4, 4.8 Hz, 2H), 1.82-1.65 (m, 2H), 1.62-1.45 (m, 1H), 1.37-1.15 (m, 1H).

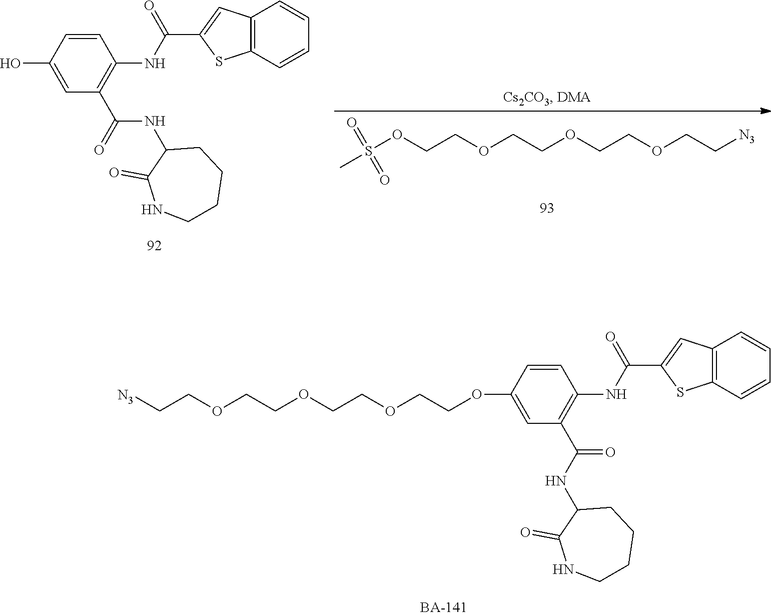 [SMILES: O=C(Nc1ccc(O)cc1C(=O)NC1CCCCNC1=O)c1cc2ccccc2s1 ; CS(=O)(=O)OCCOCCOCCOCCN=[N+]=[N-] ; [N-]=[N+]=NCCOCCOCCOCCOc1ccc(NC(=O)c2cc3ccccc3s2)c(C(=O)NC2CCCCNC2=O)c1]

The phenol 92 (0.25 g, 6.8 mmol) was dissolved/suspended in dry DMA (3 mL) and treated with mesylate 93 (0.18 g, 0.59 mmol, 1 equiv.) and powdered cesium carbonate (Oakwood; 0.4 g, 1.2 mmol, 2 equiv.). The reaction mixture was stirred at 60° C. over the weekend. LCMS data is consistent with the presence of the desired product: LCMS data: t=2.74 min, MS(+) m/z: 625.5 for [C₃₀H₃₆N₆O₇S]⁺ [M+H]⁺, 647.5 for [C₃₀H₃₅N₆NaO₇S]⁺ [M+Na]⁺; MS(−) m/z: 623.5 for [C₃₀H₃₅N₆O₇S]− [M−H]−. The mixture was poured into 150 mL of water. A very fine beige ppt formed that could not be collected by filtration. The pH of the mixture was adjusted to <6 using 10% citric acid. After sonication and aging for 20 min, the resulting precipitate was filtered, washed with water, and air-dried. NMR data showed very broad peaks. The product was dissolved in 1:1 acetonitrile/methanol. An insoluble, gray material separated; it was filtered off. The filtrate was concentrated to yield a beige solid, which was purified by silicagel chromatography (0% to 75% [3:1 ethyl acetate/ethanol] in hexanes with a 100% B flush, Biotage 25-g column), pure fractions were combined and concentrated to obtain. BA-141 (68 mg, 16%) as a beige solid. LCMS data: t=2.15 min, MS(+) m/z: 625.6 for [C₃₀H₃₆N₆O₇S]⁺ [M+H]⁺, 647.4 for [C₃₀H₃₅N₆NaO₇S]⁺ [M+Na]⁺; MS(−) m/z: 623.5 for [C₃₀H₃₅N₆O₇S]− [M−H]−. The NMR data of fraction 1 material (MDA0086-055-1) is consistent with the structure of the desired product. ¹H NMR (499 MHZ, DMSO-d₆) δ 11.94 (s, 1H), 8.71 (d, J=7.3 Hz, 1H), 8.31 (d, J=9.0 Hz, 1H), 8.11-8.01 (m, 3H), 7.87 (dd, J=7.4, 4.9 Hz, 1H), 7.53-7.41 (m, 3H), 7.20 (dd, J=9.1, 2.9 Hz, 1H), 4.72-4.63 (m, 1H), 4.18 (dd, J=6.0, 3.6 Hz, 2H), 3.78 (dd, J=3.7, 2.4 Hz, 2H), 3.65-3.51 (m, 10H), 3.39 (dd, J=5.6, 4.3 Hz, 2H), 3.31-3.21 (m, 1H), 3.10 (dt, J=13.4, 5.9 Hz, 1H), 1.97-1.86 (m, 2H), 1.83-1.67 (m, 1H), 1.66-1.54 (m, 1H), 1.31-1.11 (m, 2H).

### Example 20: BA-118/BA-118 Conjugate

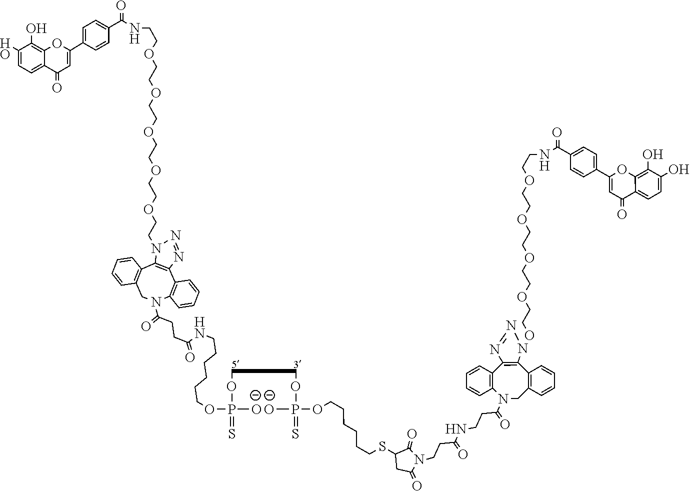

BA-118 was prepared according to the procedure described in Example 3. Subsequently, BA-118 was conjugated to an oligo sense strand according to general procedure Type III A or Type III B. MW calculated: 9327.41. MW Found: 9326.33.

### Example 21: BA-167/BA-167 Conjugate

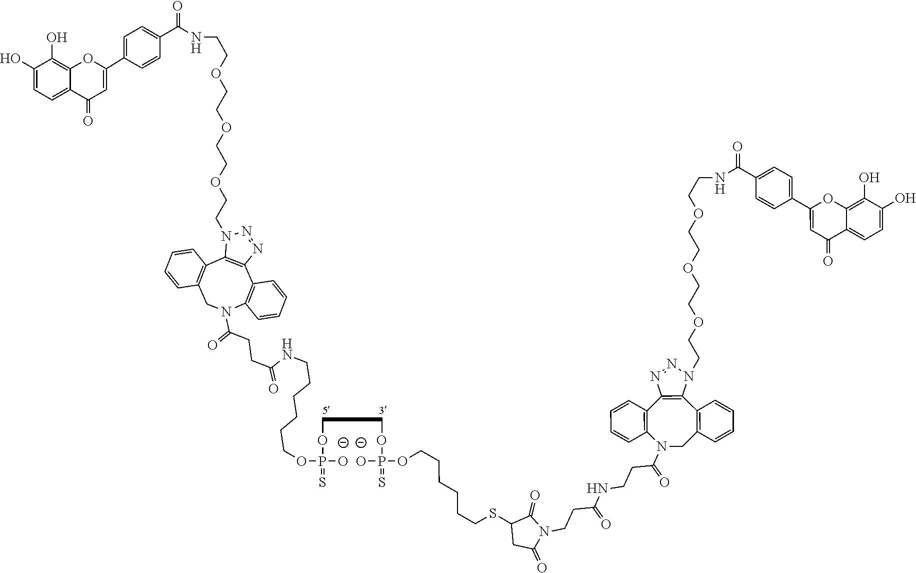

BA-167 was prepared according to the procedure described in Example 12 and conjugated to an oligo sense strand according to general procedure Type III A. MW calculated: 9150.3. MW Found: 9150.06.

### Example 22: BA-118/BA-167 Conjugate

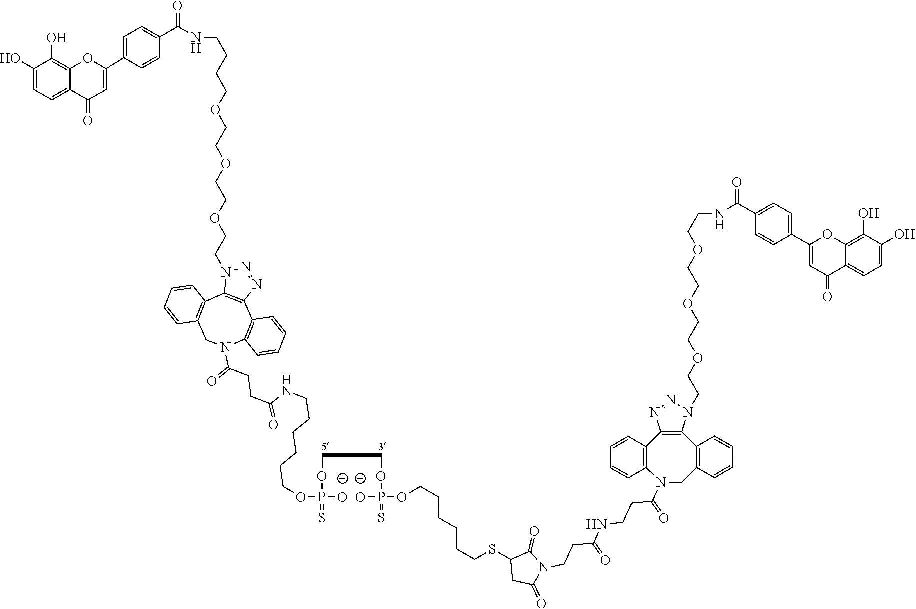

BA-118 and BA-167 were conjugated to an oligo sense strand according to general procedure Type III A. MW calculated: 9239.36, MW Found: 9238.20.

### Example 23: BA-118/BA-129 Conjugate

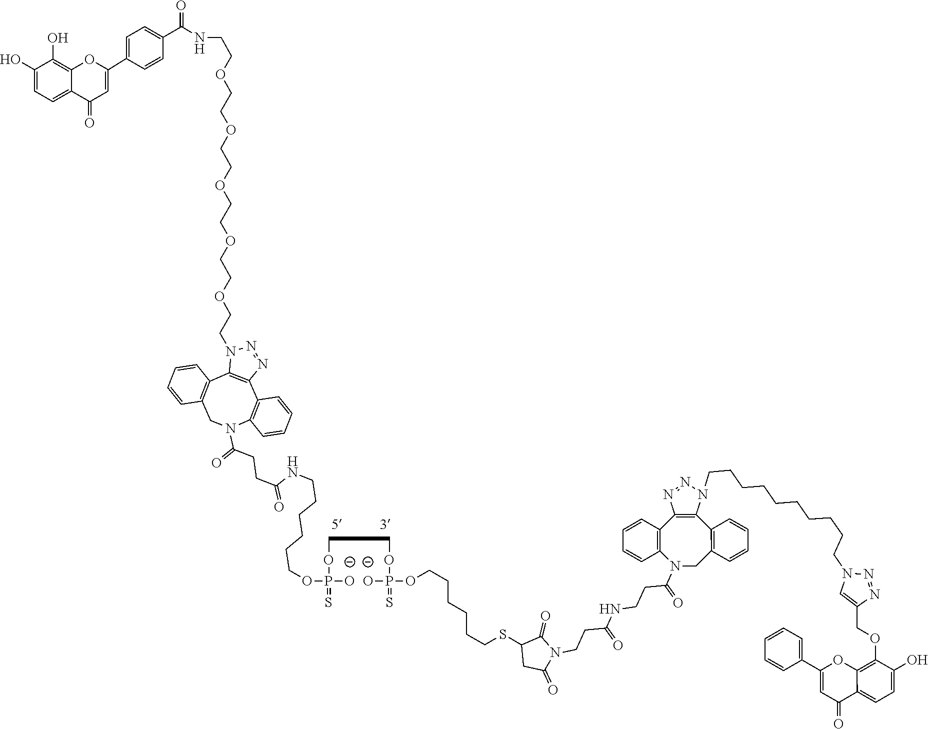

BA-118 and BA-129 were conjugated to an oligo sense strand according to general procedure Type III A. MW calculated: 9257.78. MW Found: 9256.51.

### Example 24: BA-118/BA-170 Conjugate

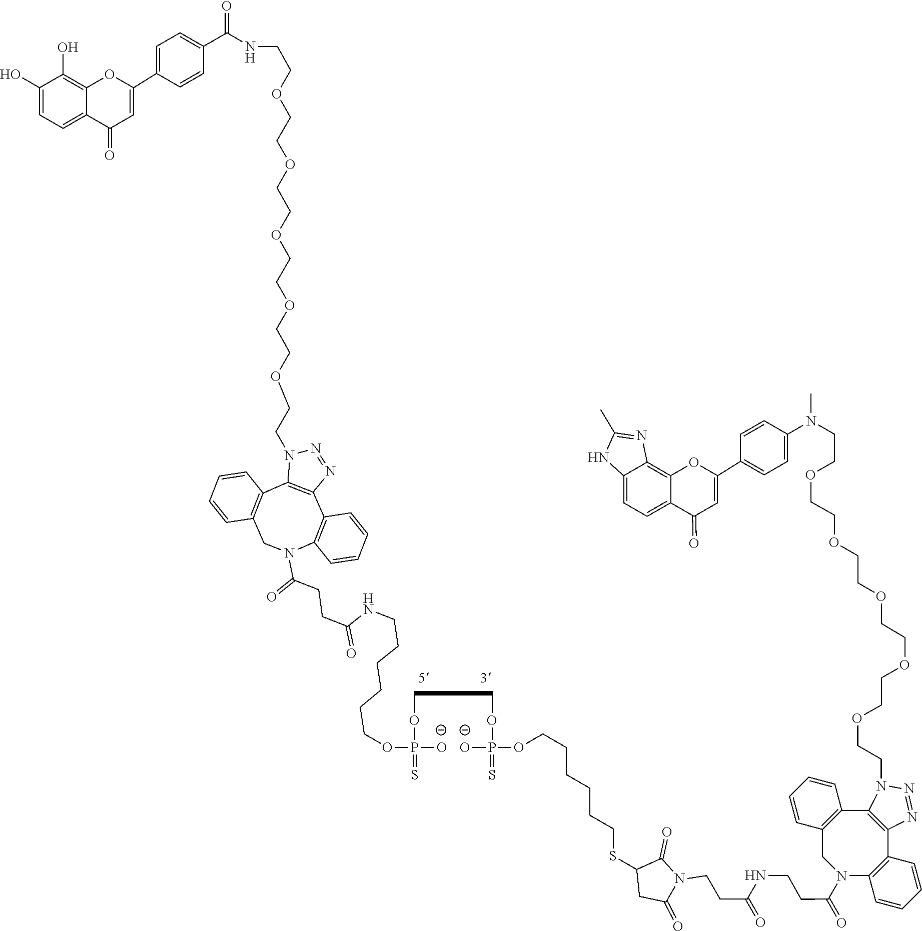

BA-118 and BA-170 were conjugated to an oligo sense strand according to general procedure Type III A. MW calculated: 9335.85. MW Found: 9334.51.

### Example 25: BA-118/BA-201 Conjugate

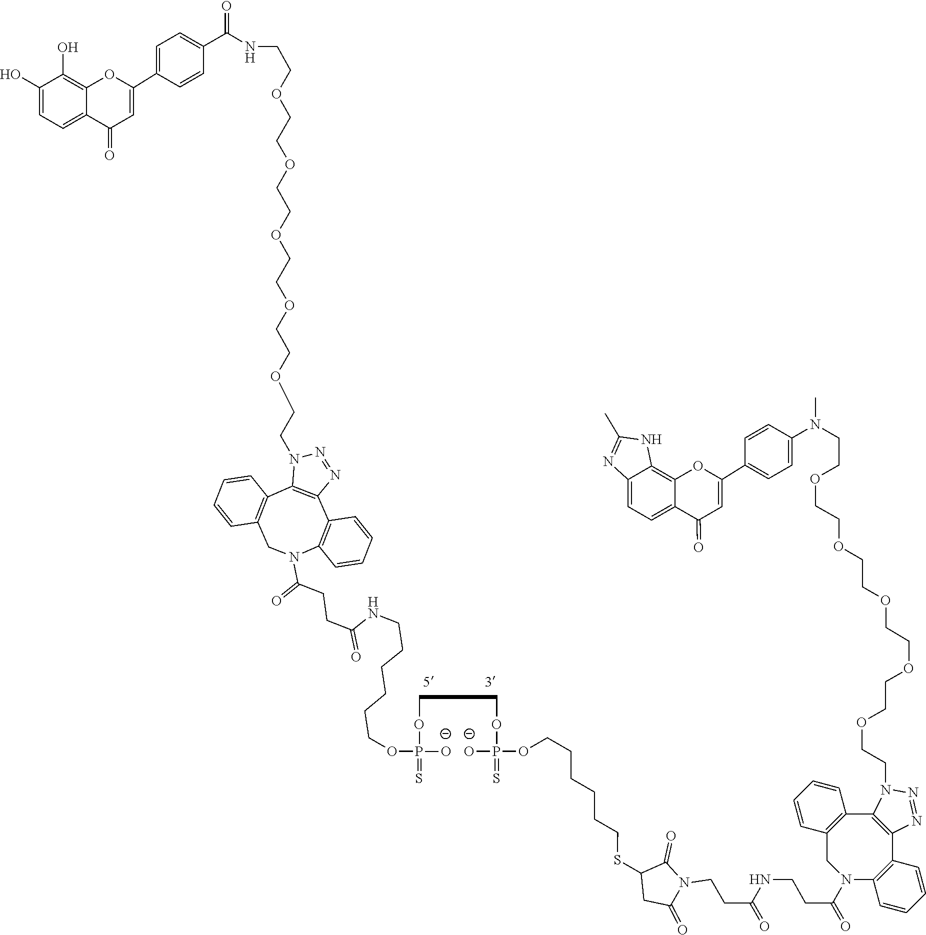

BA-118 and BA-201 were prepared according to the procedure described in Examples 4 and 14, respectively. BA-118 and BA-201 will be conjugated to an oligo sense strand according to general procedure Type III A.

### Example 26: BA-129/BA-129 Conjugate

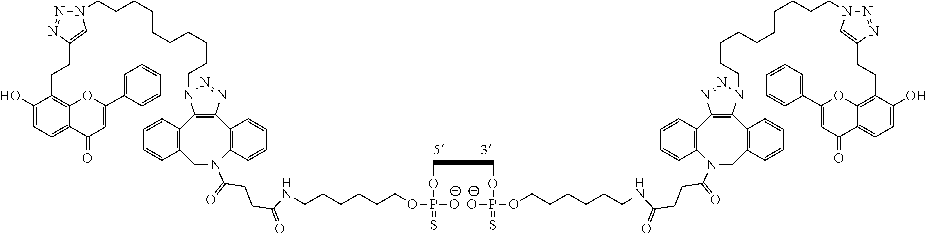

BA-129 was prepared according to the procedure described in Example 5. BA-129 will be conjugated to an oligo sense strand according to general procedure Type III A or Type III B.

### Example 27: BA-179/BA-179 Conjugate

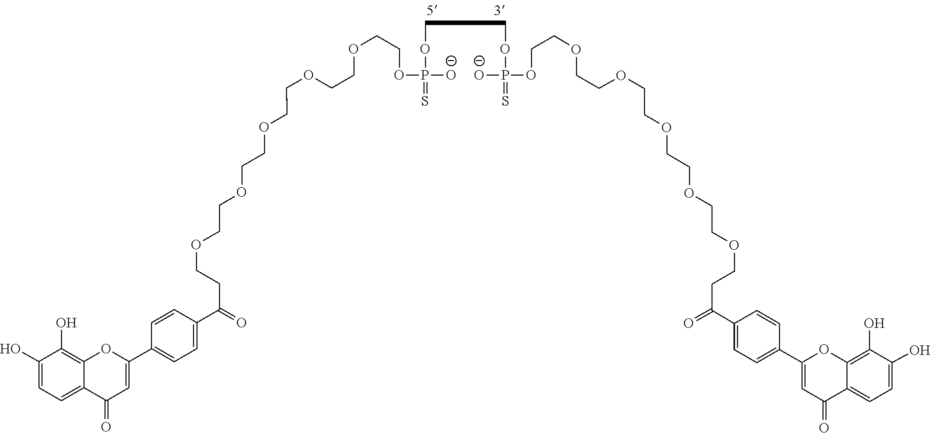

BA-179 was prepared according to the procedure described in Example 16. BA-179 will be conjugated to an oligo sense strand according to general procedure Type III A or Type III B.

### Example 28: BA-203 Conjugate

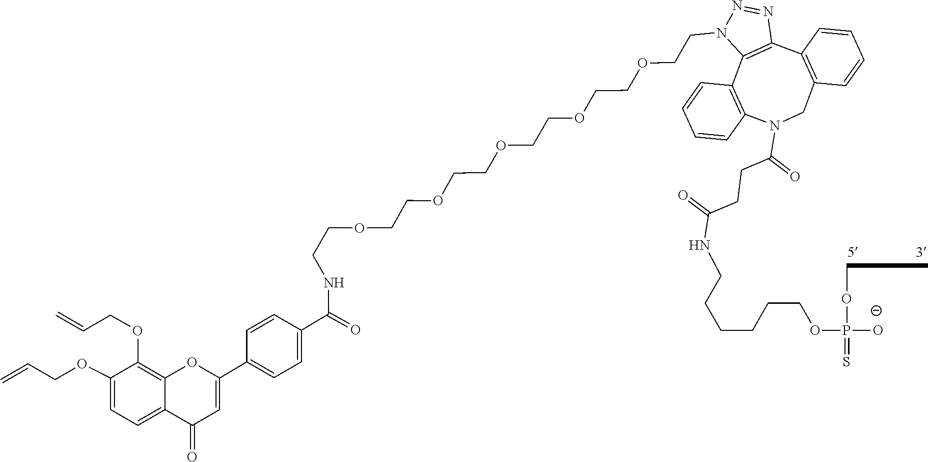

### Synthesis of BA-203

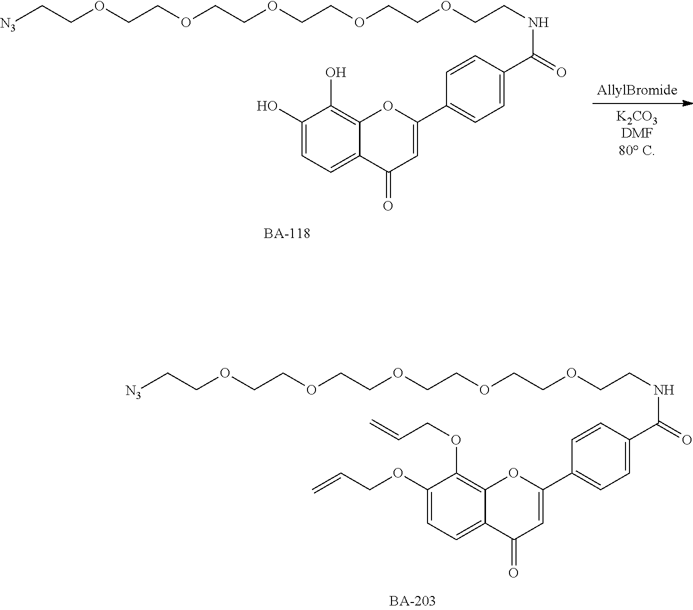 [SMILES: [N-]=[N+]=NCCOCCOCCOCCOCCOCCNC(=O)c1ccc(-c2cc(=O)c3ccc(O)c(O)c3o2)cc1 ; C=CCOc1ccc2c(=O)cc(-c3ccc(C(=O)NCCOCCOCCOCCOCCOCCN=[N+]=[N-])cc3)oc2c1OCC=C]

To a solution of diol BA-118 (200 mg, 0.34 mmol, 1eq) in 5 mL DMF, was added K₂CO₃ (94 mg, 0.68 mmol, 2 eq) followed by Allyl bromide (35.2 uL, 0.408 mmol, 1.2 eq) at RT and heated to 80° C. After 3 hr reaction went to completion by LCMS, mixture was filtered, and excess DMF was azeotroped with heptane. Purified by flash chromatography (25 g biotage 20 micron) 0 to 10% DCM/MeOH 10CV yielded desired product BA-203 (140 mg, as yellow oil 62%).

LCMS: m/z 689 (M⁺+Na). ¹H NMR (499 MHz, DMSO-d₆) δ 8.71 (t, J=5.6 Hz, 1H), 8.16 (d, J=8.2 Hz, 2H), 8.04 (d, J=8.3 Hz, 2H), 7.76 (d, J=8.9 Hz, 1H), 7.28 (d, J=9.0 Hz, 1H), 7.07 (s, 1H), 6.13 (dddt, J=24.8, 16.0, 10.4, 5.4 Hz, 2H), 5.51-5.39 (m, 2H), 5.32 (d, J=10.6 Hz, 1H), 5.27 (d, J=10.4 Hz, 1H), 4.79 (d, J=5.0 Hz, 2H), 4.70 (d, J=5.9 Hz, 2H), 3.60-3.48 (m, 20H), 3.45 (q, J=6.0 Hz, 2H), 3.37 (t, J=4.9 Hz, 2H).

BA-203 will be conjugated to an oligo sense strand according to general procedure Type III A or Type III B.

### Example 29: BA-204 and BA-205 Conjugates

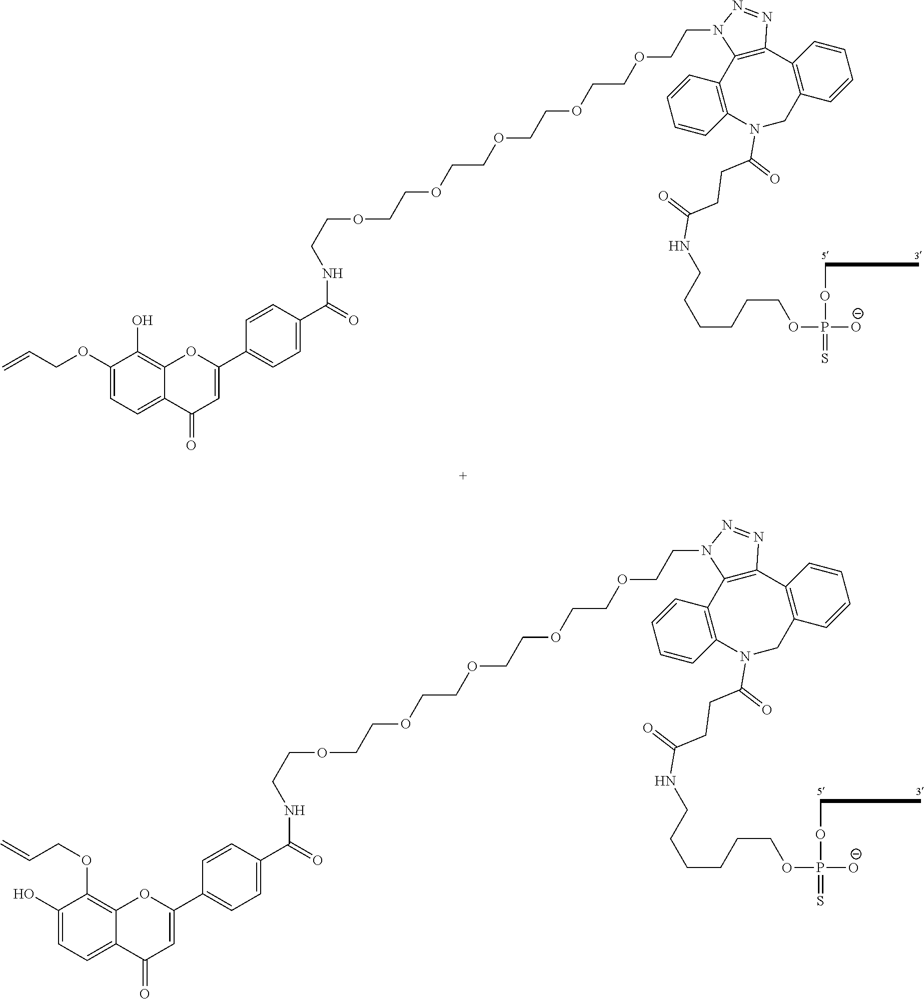

### Synthesis of BA-204 and BA-205

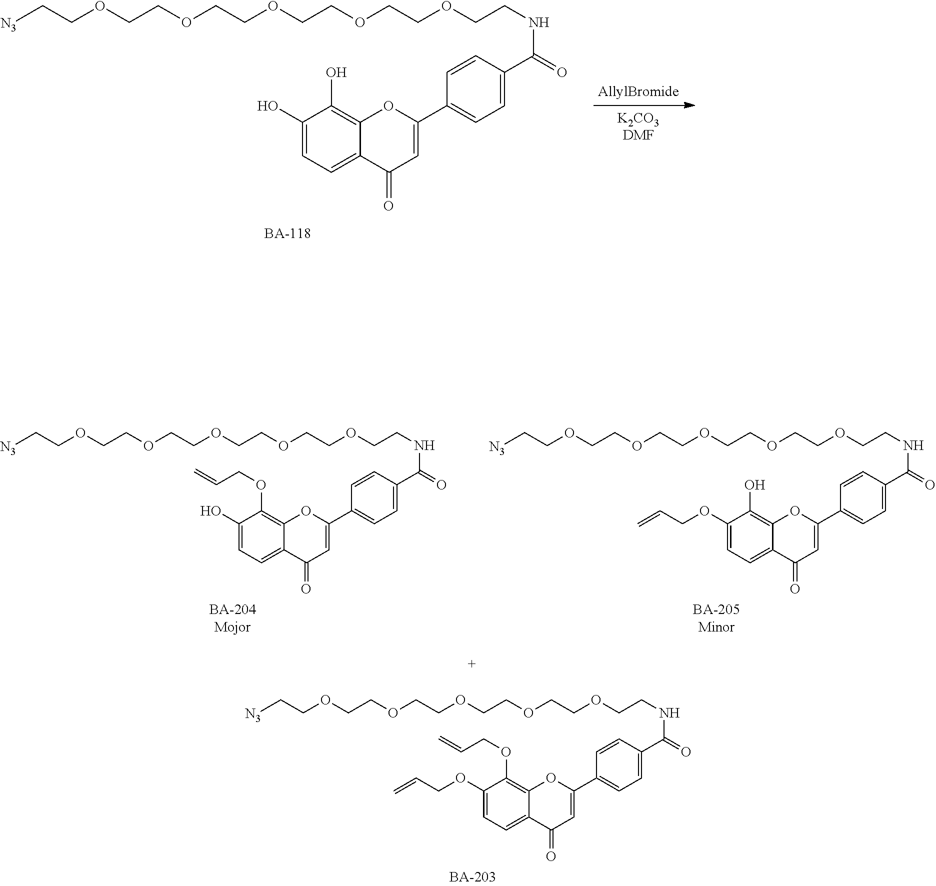 [SMILES: [N-]=[N+]=NCCOCCOCCOCCOCCOCCNC(=O)c1ccc(-c2cc(=O)c3ccc(O)c(O)c3o2)cc1 ; C=CCOc1c(O)ccc2c(=O)cc(-c3ccc(C(=O)NCCOCCOCCOCCOCCOCCN=[N+]=[N-])cc3)oc12 ; C=CCOc1ccc2c(=O)cc(-c3ccc(C(=O)NCCOCCOCCOCCOCCOCCN=[N+]=[N-])cc3)oc2c1O ; C=CCOc1ccc2c(=O)cc(-c3ccc(C(=O)NCCOCCOCCOCCOCCOCCN=[N+]=[N-])cc3)oc2c1OCC=C]

To a solution of BA-118 (350 mg, 0.596 mmol, 1 eq) in DMF (3 mL), was added Allyl bromide (72.1 mg, 0.596 mmol, 1 eq) followed by the K₂CO₃ (165 mg, 1.19 mmol, 2 eq) at room temperature, and the reaction was carefully monitored by LCMS. After 3 hours reaction shows no change. LCMS shows 50.7% conversion by LCMS with the mixtures of the BA-204 (30.9%) as major isomer, BA-205 (10.3%) as minor isomer and 9.5% Di-allylated product BA-203. Mixture was filtered, and DMF was azeotroped with heptane. BA-204 and BA-205 were separated by reverse phase flash chromatography and collected only pure fractions. Obtained 6 mg of BA-204 as well as the 4 mg of BA-205. LCMS m/z 649 (M⁺+Na).

BA-204: ¹H NMR (499 MHz, DMSO-d₆) δ 8.72 (t, J=5.6 Hz, 1H), 8.15 (d, J=8.5 Hz, 2H), 8.03 (d, J=8.5 Hz, 2H) 7.64 (d, J=8.8 Hz, 1H), 7.03 (t, J=4.4 Hz, 2H), 6.17 (ddt, J=17.4, 10.4, 5.8 Hz, 1H), 5.42 (dq, J=17.2, 1.7 Hz, 1H), 5.26 (dt, J=10.4, 1.5 Hz, 1H), 4.68 (dt, J=5.8, 1.4 Hz, 2H), 3.64-3.49 (m, 20H), 3.49-3.41 (m, 2H), 3.40-3.34 (m, 2H).

BA-205: ¹H NMR (499 MHz, DMSO-d₆) δ 8.73 (t, J=5.6 Hz, 1H), 8.24 (d, J=8.5 Hz, 1H), 8.03 (dd, J=8.5, 1.8 Hz, 2H), 7.49 (d, J=8.9 Hz, 1H), 7.20 (d, J=8.9 Hz, 1H), 7.04 (s, 1H), 6.09 (ddt, J=17.3, 10.5, 5.2 Hz, 1H), 5.47 (dq, J=17.3, 1.8 Hz, 1H), 5.30 (dq, J=10.6, 1.5 Hz, 1H), 4.78 (dt, J=5.2, 1.6 Hz, 2H), 3.61-3.43 (m, 22H), 3.40-3.34 (m, 2H).

BA-179 will be conjugated to an oligo sense strand according to general procedure Type III A or Type III B.

BA-204 and/or BA-205 will be conjugated to an oligo sense strand according to general procedure Type III A or Type III B.

### Example 30: Effect of RD2816 targeting rat CTNNB1 (Catenin Beta 1)

The compound RD2816 has the following structure (an siRNA targeting CTNNB1 having a TrkB ligand with Core structure BA-117):

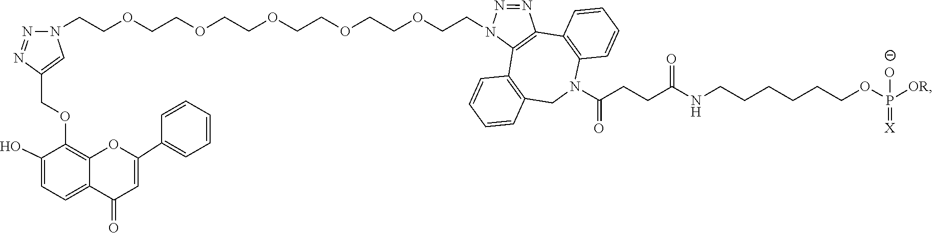

wherein X is S and R is the siRNA targeting CTNNB1 (i.e., R is the siRNA structure of compound RD2540 described below in Table 5, wherein the siRNA is attached to the TrkB targeting ligand at the 5′ end of the sense strand).

RD2816 was evaluated in an in vivo rat PD study. The animals received a single vehicle or 0.9 mg (3 mg/kg) dose via intrathecal injection on day 1 (n=3/group). Animals were observed every day for behavioral changes. Frontal Cortex was collected from half the animals on day 15 and the other half at day 29. Tissue was immediately placed in a homogenizing tube, snap frozen, and stored at −80° C. for gene expression analysis.

RNA Isolation was performed according to the RNeasy Micro Kit (Qiagen Cat #74004) instructions. Following RNA isolation, a 96-well plate was placed on ice while the qRT-PCR reaction was prepared. 2 μl of RNA was added to the reaction mixture containing 5 μl TaqMan Fast Virus 1—Step Master Mix (Thermo Fisher #44444432), 1 μl CTNNB1 TaqMan Gene Expression Assay (Thermo Fisher: Rn00584431_g1, FAM), 1 μl ACTB (VIC) TaqMan Gene Expression Assay (Thermo Fisher: Rn 00667869_m1, VIC) and 11 μl RT-PCR grade nuclease-free water in a MicroAmp Optical 96-well plate (0.2 mL). qPCR was performed using a QuantStudio3 qPCR machine with the following cycles: 50° C. for 1 minute, 95° C. for 20 seconds, 40 cycles at 95° C. for 15 seconds, and 60° C. for 1 minute. Results are presented in the tables below as percent inhibition of CTNNB1, relative to vehicle control.

TABLE 1
Average CTNNB1 Inhibition

| Compound | Day 15 | Day 29 |
|---|---|---|
| RD2816 | 35% | 50% |

### Example 31: Effect of RD2822 Targeting Rat CTNNB1

The compound RD2822 has the following structure: (an siRNA targeting CTNNB1 having a TrkB ligand with Core structure BA-118):

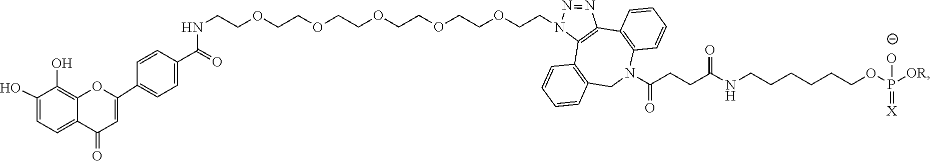

wherein X is S and R is the siRNA targeting CTNNB1 (i.e., R is the siRNA structure of compound RD2540 described in Table 5 below, wherein the siRNA is attached to the TrkB targeting ligand at the 5′ end of the sense strand).

RD2822 was evaluated in an in vivo rat PD study carried out as described in Example 30. Results are presented in Table 2 below as percent inhibition of CTNNB1, relative to vehicle control.

TABLE 2
Average CTNNB1 Inhibition

| Compound | Day 15 | Day 29 |
|---|---|---|
| RD2822 | 72% | 51% |

### Example 32: Effect of RD2822 Targeting Rat CTNNB1 in Various Brain Regions

The compound RD2822 as described above was evaluated in an in vivo rat PD study. The animals received a single vehicle or 0.9 mg (3 mg/kg) dose via intrathecal injection on day 1 (n=3/group). Animals were observed every day for behavioral changes. Brain regions were collected from half the animals on day 15 and the other half on day 29. Tissue was immediately placed in homogenizing tube, snap frozen, then kept at −80° C. for gene expression analysis.

RNA Isolation and qPCR was performed as described in Example 30 above. Results are presented in Table 3 below as percent inhibition of CTNNB1, relative to vehicle control.

TABLE 3
Average CTNNB1 Inhibition by Compound RD2822

| Site | Day 15 | Day 29 |
|---|---|---|
| Striatum | 48% | 50% |
| Cerebellum | 62% | 65% |
| Brain Stem | 68% | 78% |
| Hippocampus | 71% | 72% |
| Frontal Cortex | 55% | 59% |
| Spinal Cord | 72% | 70% |

### Example 33: Effect of RD2540 Targeting Rat CTNNB1

The compound RD2540 has the structure described in Table 5. RD2540 is the siRNA used in Examples 30-32 above and is not conjugated to a targeting ligand. The activity of RD2540 was tested as a comparison to the targeting ligand-conjugated compound tested above.

TABLE 4
Chemical Nomenclature

| Abbreviation | Structure |
|---|---|
| ‘v’ | 5′-vinylphosphonate modification |
| ‘m’ | 2′-O-methyl sugar modification (e.g., mA, mG, mC, mU) |
| ‘f’ | 2′-F sugar modification (e.g., fA, fG, fC, fU) |
| ‘*’ | Phosphorothioate internucleoside linkage |
| ‘dQ’ | Inverted abasic deoxyribose |

TABLE 5
Unconjugated Parent Compound

| Com- | | Ref ID | SEQ ID |
|---|---|---|---|
| pound | Modified Strands (5′-3′) | NO: | NO: |
| RD2540 | (vU)*(fU)*(mU)(mC)(mG) | IA0883 | 1 |
| | (fA)(mA)(fU)(fC)(mA) | | |
| | (mA)(mU)(mC)(fC)(mA) | | |
| | (fA)(mC)(mA)(mG)(mU) | | |
| | (mA)*(mG)*(mC) | | |
| | (mU)*(mA)*(mC)(mU)(mG) | IS1096 | 2 |
| | (mU)(fU)(mG)(fG)(fA) | | |
| | (fU)(mU)(mG)(mA)(mU) | | |
| | (mU)(mC)(mG)(mA) | | |
| | (mA)*(mA)*(dQ) | | |

RD2540 was evaluated in an in vivo rat PD study carried out as described in Example 30. Results are presented in Table 6 below as percent inhibition of CTNNB1, relative to vehicle control.

TABLE 6
Average CTNNB1 Inhibition

| Compound | Day 15 | Day 29 |
|---|---|---|
| RD2540 | 31% | 13% |

### Example 34: Effect of RD3072 Targeting Rat SOD1 in Various Brain Regions

The Compound RD3072 has the following structure (an siRNA targeting SOD1 having a TrkB ligand with Core structure BA-118)

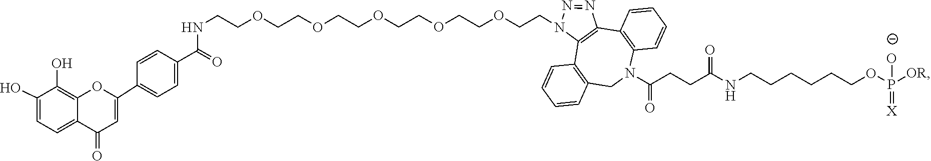

wherein X is S and R is an siRNA targeting SOD1 (i.e., R is the siRNA provided in Table 7 below, wherein the siRNA is attached to the TrkB targeting ligand at the 5′ end of the sense strand).

RD3072 was evaluated in an in vivo rat PD study. The animals received a single vehicle or 0.9 mg (3 mg/kg) dose via intrathecal injection on day 1 (n=3/group). Animals were observed every day for behavioral changes. Brain regions were collected on day 15, day 29, day 43, day 57, day 73, day 83, day 98, day 126, or day 169, and tissue was immediately placed in homogenizing tube, snap frozen, then kept at −80° C. for gene expression analysis.

RNA Isolation and qPCR was performed as described in Example 30 above, with the exception that instead of rat CTNNB1 TaqMan Gene Expression Assay, the rat SOD1 TaqMan Gene Expression Asay (Thermo Fisher: Rn00566938_ml, FAM) was used. Results are presented in Table 8 below as percent inhibition of SOD1, relative to vehicle control.

TABLE 7
Compound RD3072 (also see chemical nomenclature
provided in Table 4 above)

| Com- | | Ref ID | SEQ ID |
|---|---|---|---|
| pound | Modified Strands (5′-3′) | NO: | NO: |
| RD3072 | (vU)*(fU)*(mU)(mA)(mG)(fA) | IA1096 | 3 |
| | (mG)(fU)(fG)(mA)(mG) | | |
| | (mG)(mA)(fU)(mU)(fA)(mA) | | |
| | (mA)(mA)(mU)(mG)*(mA) | | |
| | *(mG) | | |
| | (TrkB_Ligand)*(mC)*(mA) | IS1295 | 4 |
| | (mU)(mU)(mU)(mU)(fA) | | |
| | (mA)(fU)(fC)(fC)(mU)(mC) | | |
| | (mA)(mC)(mU)(mC)(mU) | | |
| | (mA)*(mA)*(mA) | | |

TABLE 8
Average SOD1 Inhibition by Compound RD3072

| | Day | Day | Day | Day | Day | Day | Day | Day | Day |
|---|---|---|---|---|---|---|---|---|---|
| Brain Region | 15 | 29 | 43 | 57 | 73 | 83 | 98 | 126 | 169 |
| Striatum | 60% | 59% | 57% | 47% | 35% | 20% | 38% | 13% | −3% |
| Hippocampus | 89% | 89% | 79% | 81% | 77% | 52% | 55% | 23% | 15% |
| Frontal Cortex | 91% | 87% | 83% | 86% | 43% | 51% | 38% | 24% | 10% |
| Temporal Cortex | 90% | 86% | 88% | 88% | 72% | 55% | 67% | 44% | 42% |
| Cerebellum | 85% | 86% | 85% | 83% | 78% | 70% | 66% | 57% | 42% |
| Brainstem | 87% | 92% | 86% | 83% | 82% | 65% | 82% | 27% | 24% |
| Cervical Spinal Cord | 91% | 90% | 88% | 92% | 81% | 74% | 61% | 57% | 47% |
| Thoracic Spinal Cord | 89% | 85% | 86% | 88% | 79% | 73% | 47% | 48% | 41% |
| Lumbar Spinal Cord | 87% | 89% | 90% | 87% | 76% | 72% | 57% | 64% | 43% |

### Example 35: Effect of Compound a Targeting Rat SOD1 in Various Brain Regions

Compound A has the following structure (an siRNA targeting SOD1 having a TrkB ligand with Core structure BA-129):

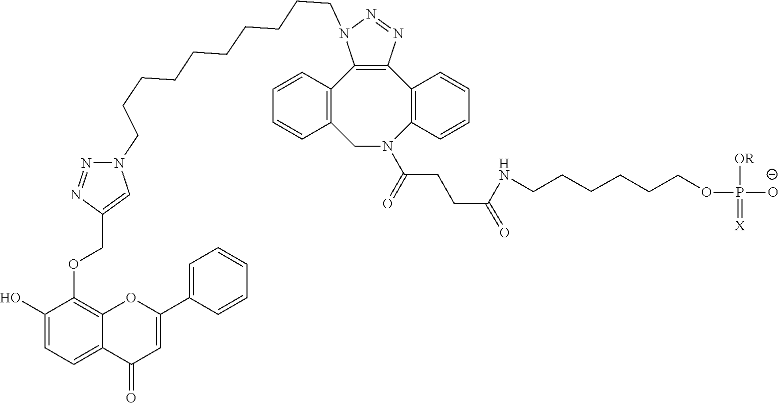

wherein X is S and R is the siRNA targeting SOD1 (i.e., R is attached at the 5′ end of the sense strand).

Compound A as described above was evaluated in an in vivo rat PD study. The animals received a single vehicle or 0.9 mg (3 mg/kg) dose via intrathecal injection on day 1 (n=3/group). Animals were observed every day for behavioral changes. Brain regions were collected on day 15. Tissue was immediately placed in homogenizing tube, snap frozen, then kept at −80° C. for gene expression analysis.

RNA Isolation and qPCR was performed as described in Example 30 above. Results are presented in Table 9 below as percent inhibition of SOD1, relative to vehicle control.

TABLE 9
Average SOD1 Inhibition by Compound A

| Site | Day 15 |
|---|---|
| Striatum | 50% |
| Hippocampus | 14% |
| Frontal Cortex | 78% |

### Example 36: Effect of Compound B Targeting Rat SOD1 in Various Brain Regions

Compound B has the following structure (an siRNA targeting SOD1 having a TrkB ligand with Core structure BA-132):

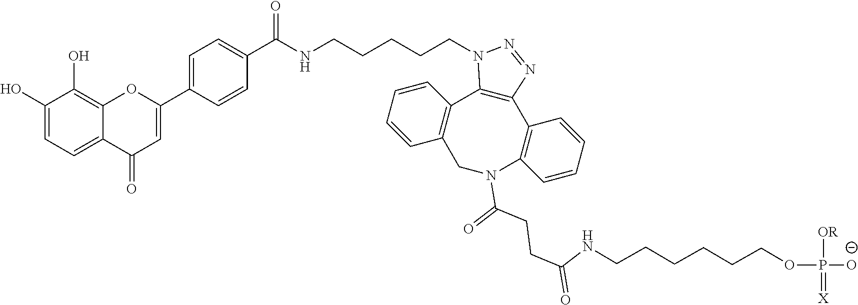

wherein X is S and R is the siRNA targeting SOD1 (i.e., R is attached at the 5′ end of the sense strand).

Compound B as described above was evaluated in an in vivo rat PD study. The animals received a single vehicle or 0.9 mg (3 mg/kg) dose via intrathecal injection on day 1 (n=3/group). Animals were observed every day for behavioral changes. Brain regions were collected on day 15. Tissue was immediately placed in homogenizing tube, snap frozen, then kept at −80° C. for gene expression analysis.

RNA Isolation and qPCR was performed as described in Example 30 above. Results are presented in Table 10 below as percent inhibition of SOD1, relative to vehicle control.

TABLE 10
Average SOD1 Inhibition by Compound Compound B

| Site | Day 15 |
|---|---|
| Striatum | 61% |
| Hippocampus | 68% |
| Frontal Cortex | 78% |

### Example 37: Effect of Compound C Targeting Rat SOD1 in Various Brain Regions

Compound C has the following structure (an siRNA targeting SOD1 having a TrkB ligand with Core structure BA-144):

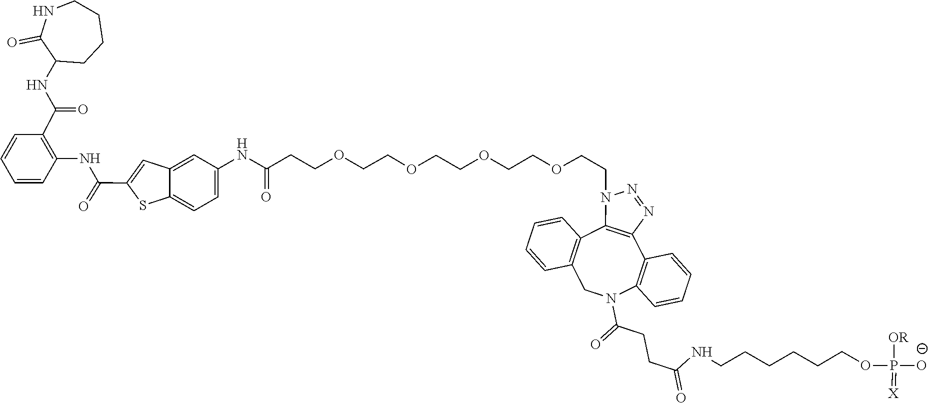

wherein X is S and R is the siRNA targeting SOD1 (i.e., R is attached at the 5′ end of the sense strand).

Compound C as described above was evaluated in an in vivo rat PD study. The animals received a single vehicle or 0.9 mg (3 mg/kg) dose via intrathecal injection on day 1 (n=3/group). Animals were observed every day for behavioral changes. Brain regions were collected on day 15, Day 29, or Day 43. Tissue was immediately placed in homogenizing tube, snap frozen, then kept at −80° C. for gene expression analysis.

RNA Isolation and qPCR was performed as described in Example 30 above. Results are presented in Table 11 below as percent inhibition of SOD1, relative to vehicle control.

TABLE 11
Average SOD1 Inhibition by Compound C

| Brain Region | Day 15 | Day 29 | Day 43 |
|---|---|---|---|
| Striatum | 30% | 35% | 71% |
| Hippocampus | 64% | 75% | 75% |
| Frontal Cortex | 74% | 62% | 82% |
| Temporal Cortex | 72% | 79% | 69% |
| Cerebellum | 79% | 76% | 75% |
| Brainstem | 81% | 87% | 94% |
| Cervical Spinal Cord | 84% | 83% | 85% |
| Thoracic Spinal Cord | 87% | 86% | 77% |
| Lumbar Spinal Cord | 74% | 89% | 84% |

### Example 38: Effect of Compound D Targeting Rat SOD1 in Various Brain Regions

Compound D has the following structure (an siRNA targeting SOD1 having a TrkB ligand with Core structure BA-133):

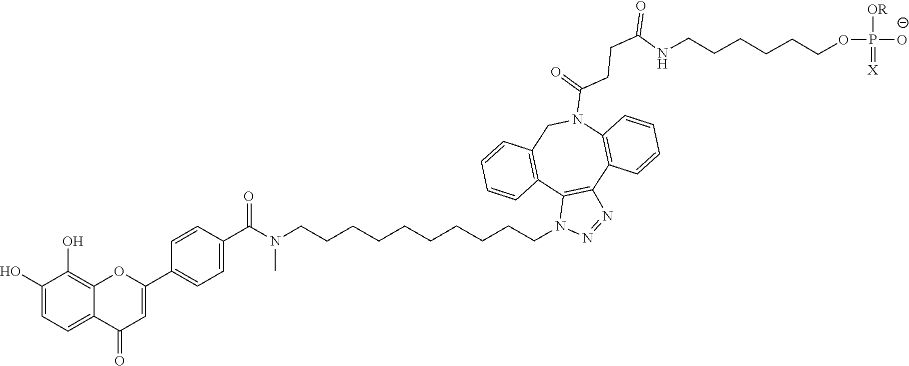

wherein X is S and R is the siRNA targeting SOD1 (i.e., R is attached at the 5′ end of the sense strand).

Compound D as described above was evaluated in an in vivo rat PD study. The animals received a single vehicle or 0.9 mg (3 mg/kg) dose via intrathecal injection on day 1 (n=3/group). Animals were observed every day for behavioral changes. Brain regions were collected on day 15 or day 29. Tissue was immediately placed in homogenizing tube, snap frozen, then kept at −80° C. for gene expression analysis.

RNA Isolation and qPCR was performed as described in Example 30 above. Results are presented in Table 12 below as percent inhibition of SOD1, relative to vehicle control.

TABLE 12
Average SOD1 Inhibition by Compound D

| Brain Region | Day 15 | Day 29 |
|---|---|---|
| Striatum | 43% | 40% |
| Hippocampus | 67% | 44% |
| Frontal Cortex | 59% | 57% |
| Temporal Cortex | 71% | 63% |
| Cerebellum | 72% | 70% |
| Brainstem | 88% | 84% |
| Cervical Spinal Cord | 76% | 86% |
| Thoracic Spinal Cord | 81% | 85% |
| Lumbar Spinal Cord | 82% | 90% |

### Example 39: Effect of Compound E Targeting Rat SOD1 in Various Brain Regions

Compound E has the following structure (an siRNA targeting SOD1 having a TrkB ligand with Core structure BA-137):

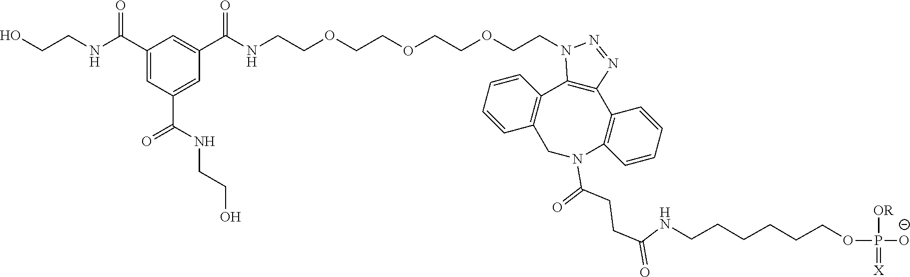

wherein X is S and R is the siRNA targeting SOD1 (i.e., R is attached at the 5′ end of the sense strand).

Compound E as described above was evaluated in an in vivo rat PD study. The animals received a single vehicle or 0.9 mg (3 mg/kg) dose via intrathecal injection on day 1 (n=3/group). Animals were observed every day for behavioral changes. Brain regions were collected on day 15, day 29, or day 43. Tissue was immediately placed in homogenizing tube, snap frozen, then kept at −80° C. for gene expression analysis.

RNA Isolation and qPCR was performed as described in Example 30 above. Results are presented in Table 13 below as percent inhibition of SOD1, relative to vehicle control.

TABLE 13
Average SODI Inhibition by Compound E

| Brain Region | Day 15 | Day 29 | Day 43 |
|---|---|---|---|
| Striatum | 39% | 24% | 63% |
| Hippocampus | 86% | 83% | 68% |
| Frontal Cortex | 47% | 79% | 82% |
| Temporal Cortex | 84% | 88% | 72% |
| Cerebellum | 73% | 71% | 73% |
| Brainstem | 88% | 86% | 93% |
| Cervical Spinal Cord | 86% | 84% | 83% |
| Thoracic Spinal Cord | 79% | 89% | 70% |
| Lumbar Spinal Cord | 84% | 89% | 83% |

### Example 40: Effect of Compound F Targeting Rat CTNNB1

Compound F has the following structure (an siRNA targeting CTNNB1 having a TrkB ligand with Core structure BA-129):

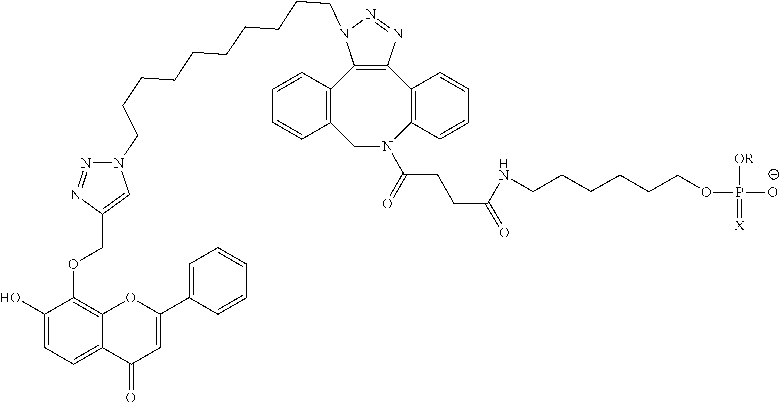

wherein X is S and R is the siRNA targeting CTNNB1 (i.e., R is attached at the 5′ end of the sense strand).

Compound F was evaluated in an in vivo rat PD study carried out as described in Example 30. Results are presented in Table 14 below as percent inhibition of CTNNB1, relative to vehicle control.

TABLE 14
Average CTNNB1 Inhibition

| Compound | Day 15 | Day 29 |
|---|---|---|
| F | 53% | 63% |

### Example 41: Effect of Compound G Targeting Target a in Various Brain Regions

Compound G has the following structure (an siRNA targeting Target A having a TrkB ligand with Core structure BA-118):

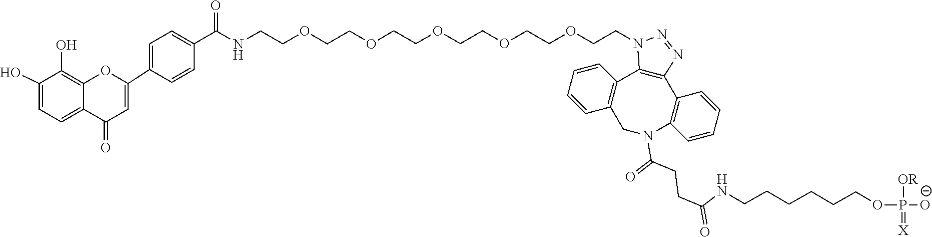

wherein X is S and R is the siRNA targeting Target A (i.e., R is attached at the 5′ end of the sense strand).

Compound G was evaluated in an in vivo human Target A transgenic mice PD study. The animals received a single vehicle or 0.2 mg (10 mg/kg) dose via intracerebroventricular injection on day 1 (n=3/group). Animals were observed every day for behavioral changes. Brain regions were collected on day 15, and tissue was immediately placed in homogenizing tube, snap frozen, then kept at −80° C. for gene expression analysis.

RNA Isolation and qPCR was performed as described in Example 30 above, with the exceptions that the instead of rat CTNNB1 TaqMan Gene Expression Assay, the human Target A TaqMan Gene Expression Assay (Thermo Fisher) was used; instead of rat ACTB (VIC) TaqMan Gene Expression Assay, the mouse GAPDH TaqMan Gene Expression Assay (Thermo Fisher: Mm99999915_g1, VIC) was used. Results are presented in Table 15 below as percent inhibition of Target A, relative to vehicle control.

TABLE 15
Average Target A Inhibition by Compound G

| Brain Region | Day 15 |
|---|---|
| Cortex | 56% |
| Hippocampus | 62% |
| Cerebellum | 62% |
| Striatum | 60% |
| Cervical Spinal Cord | 57% |

### Example 42: Effect of Compound H Targeting Target a in Various Brain Regions

Compound H has the following structure (an siRNA targeting Target A having a TrkB ligand with Core structure BA-167):

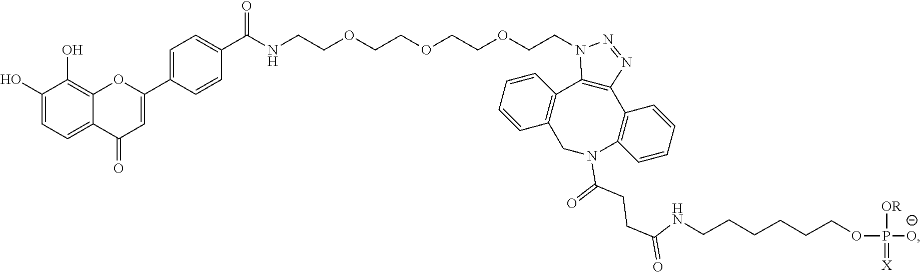

wherein X is S and R is the siRNA targeting Target A (i.e., R is attached at the 5′ end of the sense strand).

Compound H was evaluated in an in vivo Target A transgenic mice PD study carried out as described in Example 41. Results are presented in Table 16 below as percent inhibition of Target A, relative to vehicle control.

TABLE 16
Average Target A Inhibition by Compound H

| Brain Region | Day 15 |
|---|---|
| Cortex | 61% |
| Hippocampus | 73% |
| Cerebellum | 57% |
| Striatum | 71% |
| Cervical Spinal Cord | 69% |

### Example 43: Effect of Compound I Targeting Target a in Various Brain Regions

Compound I has the following structure (an siRNA targeting Target A having a TrkB ligand with Core structure BA-169):

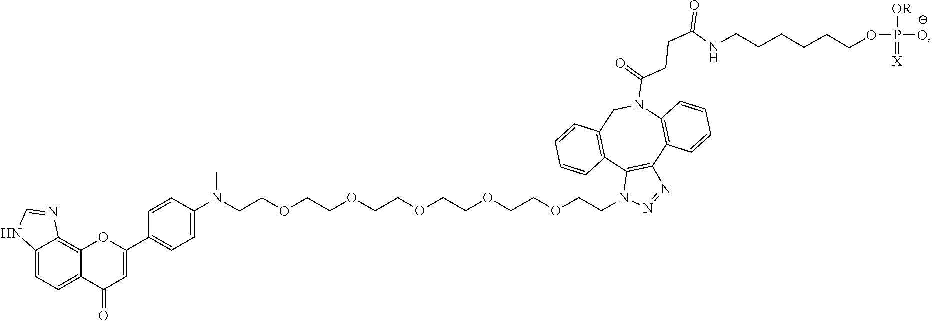

wherein X is S and R is the siRNA targeting Target A (i.e., R is attached at the 5′ end of the sense strand).

Compound I was evaluated in an in vivo human Target A transgenic mice PD study carried out as described in Example 41. Results are presented in Table 17 below as percent inhibition of Target A, relative to vehicle control.

TABLE 17
Average Target A Inhibition by Compound I

| Brain Region | Day 15 |
|---|---|
| Cortex | 54% |
| Hippocampus | 66% |
| Cerebellum | 44% |
| Striatum | 66% |
| Cervical Spinal Cord | 66% |

### Example 44: Effect of Compound J Targeting Target a in Various Brain Regions

Compound J has the following structure (an siRNA targeting Target A having a TrkB ligand with Core structure BA-170):

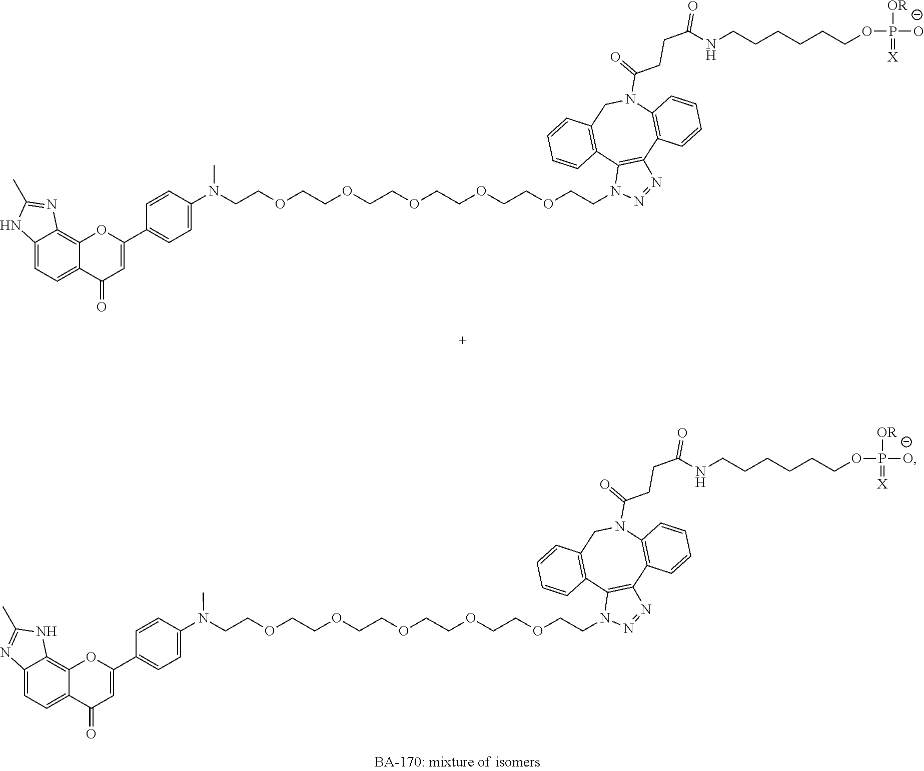

wherein X is S and R is the siRNA targeting Target A (i.e., R is attached at the 5′ end of the sense strand).

Compound J was evaluated in an in vivo human Target A transgenic mice PD study carried out as described in Example 41. Results are presented in Table 18 below as percent inhibition of Target A, relative to vehicle control.

TABLE 18
Average Target A Inhibition by Compound J

| Brain Region | Day 15 |
|---|---|
| Cortex | 68% |
| Hippocampus | 68% |
| Cerebellum | 45% |
| Striatum | 67% |
| Cervical Spinal Cord | 68% |

### Example 45: Effect of Compound K Targeting Target a in Various Brain Regions

Compound K has the following structure (an siRNA targeting Target A having a TrkB ligand with Core structure BA-173):

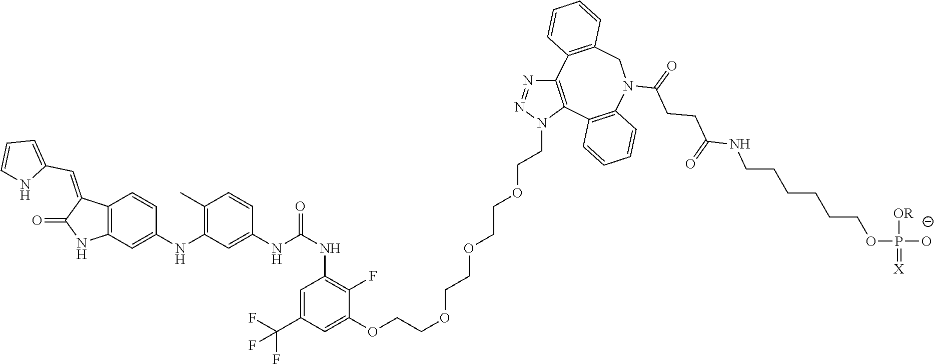

wherein X is S and R is the siRNA targeting Target A (i.e., R is attached at the 5′ end of the sense strand).

Compound K was evaluated in an in vivo human Target A transgenic mice PD study carried out as described in Example 41. Results are presented in Table 19 below as percent inhibition of Target A, relative to vehicle control.

TABLE 19
Average Target A Inhibition by Compound K

| Brain Region | Day 15 |
|---|---|
| Cortex | 27% |
| Hippocampus | 49% |
| Cerebellum | 25% |
| Striatum | 42% |
| Cervical Spinal Cord | 73% |

### Example 46: Effect of Compound L Targeting Target a in Various Brain Regions

Compound L has the following structure (an siRNA targeting Target A having a TrkB ligand with Core structure BA-118 attached at the 5′ and 3′ ends of the sense strand of the SiRNA):

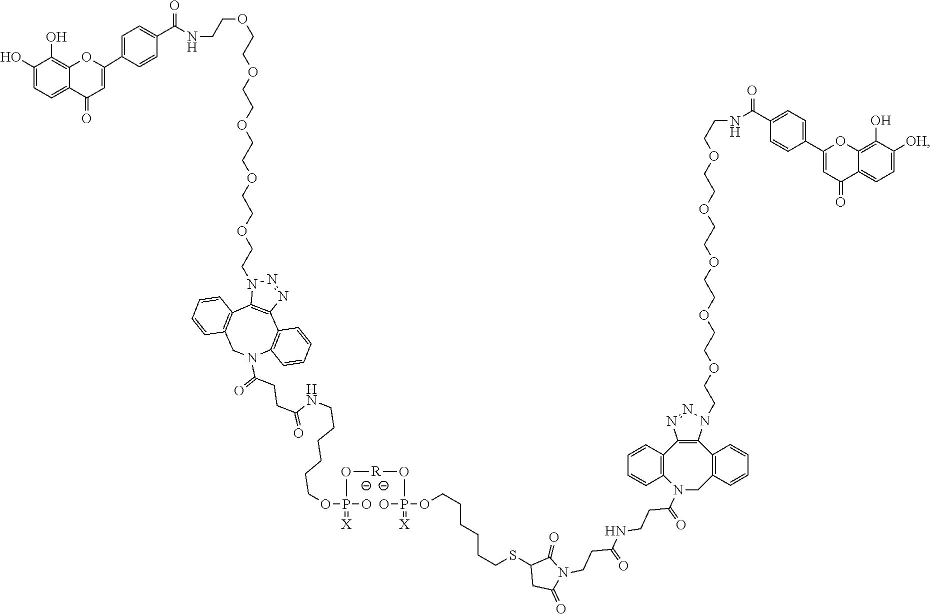

wherein X is S and R is the siRNA targeting Target A (i.e., R is attached to the TrkB ligand at the 5′ and 3′ end of the sense strand).

Compound L was evaluated in an in vivo human Target A transgenic mice PD study carried out as described in Example 41. Results are presented in Table 20 below as percent inhibition of Target A, relative to vehicle control.

TABLE 20
Average Target A Inhibition by Compound L

| Brain Region | Day 15 |
|---|---|
| Cortex | 75% |
| Hippocampus | 78% |
| Cerebellum | 64% |
| Striatum | 74% |
| Cervical Spinal Cord | 74% |

### Example 47: Effect of Compound M Targeting Target a in Various Brain Regions

Compound M has the following structure (an siRNA targeting Target A having a TrkB ligand with Core structure BA-129):

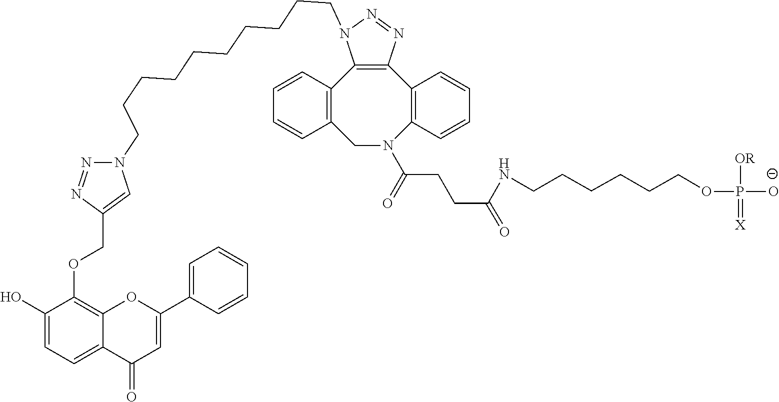

wherein X is S and R is the siRNA targeting Target A (i.e., R is attached at the 5′ end of the sense strand).

Compound M was evaluated in an in vivo human Target A transgenic mice PD study carried out as described in Example 41. Results are presented in Table 21 below as percent inhibition of Target A, relative to vehicle control.

TABLE 21
Average Target A Inhibition by Compound M

| Brain Region | Day 15 |
|---|---|
| Cortex | 69% |
| Hippocampus | 74% |
| Cerebellum | 51% |
| Striatum | 74% |
| Cervical Spinal Cord | 17% |

### Example 48: Effect of Compound N Targeting Target a in Various Brain Regions

Compound N has the following structure (an siRNA targeting Target A having TrkB ligands with Core structures BA-118 and BA-129 attached at the 5′ and 3′ ends of the sense strand, respectively):

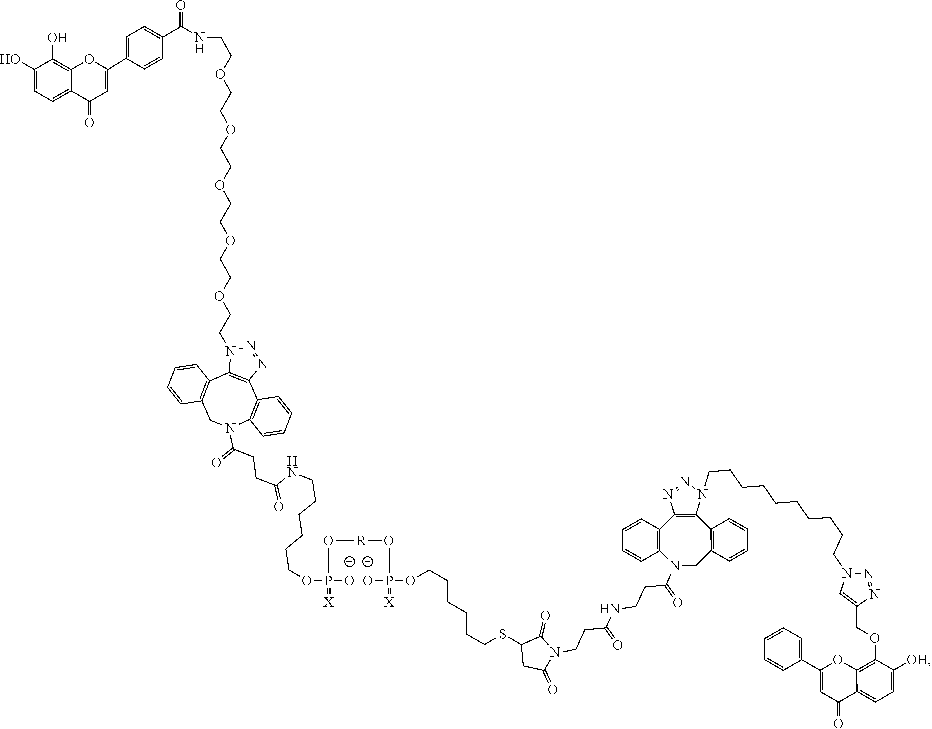

wherein X is S and R is the siRNA targeting Target A (i.e., R is attached to the TrkB ligands with Core structures BA-118 and BA-129 at the 5′ and 3′ ends of the sense strand, respectively).

Compound N was evaluated in an in vivo human Target A transgenic mice PD study carried out as described in Example 41. Results are presented in Table 22 below as percent inhibition of Target A, relative to vehicle control.

TABLE 22
Average Target A Inhibition by Compound N

| Brain Region | Day 15 |
|---|---|
| Cortex | 55% |
| Hippocampus | 74% |
| Cerebellum | 68% |
| Striatum | 67% |
| Cervical Spinal Cord | 74% |

### Example 49: Effect of Compound O Targeting Target a in Various Brain Regions

Compound O has the following structure (an siRNA targeting Target A having the following TrkB ligands with Core structures BA-118 and BA167 attached at the 5′ and 3′ ends of the sense strand, respectively):

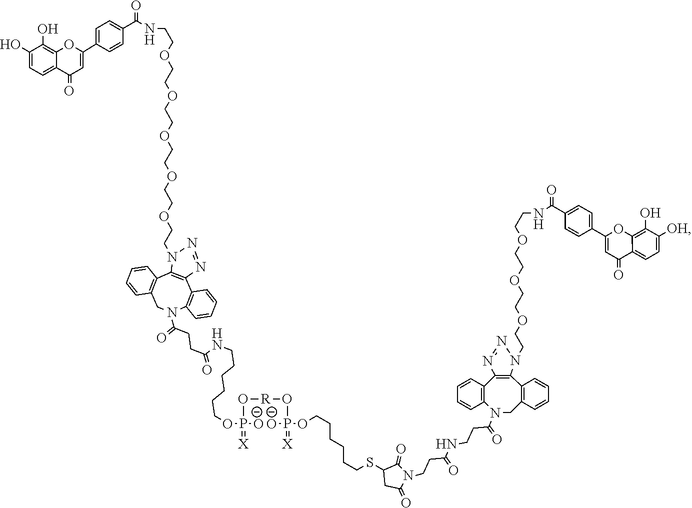

wherein X is S and R is the siRNA targeting Target A (i.e., R is attached to the TrkB ligands with Core structures BA-118 and BA-167 at the 5′ and 3′ ends of the sense strand, respectively). Compound O was evaluated in an in vivo human Target A transgenic mice PD study carried out as described in Example 41. Results are presented in Table 23 below as percent inhibition of Target A, relative to vehicle control.

TABLE 23
Average Target A Inhibition by Compound O

| Brain Region | Day 15 |
|---|---|
| Cortex | 76% |
| Hippocampus | 64% |
| Cerebellum | 77% |
| Striatum | 67% |
| Cervical Spinal Cord | 69% |

### Example 50: Effect of Compound P Targeting Target a in Various Brain Regions

Compound P has the following structure (an siRNA targeting Target A having a TrkB ligand with Core structure BA-167 attached at both the 5′ and 3′ end of the sense strand):

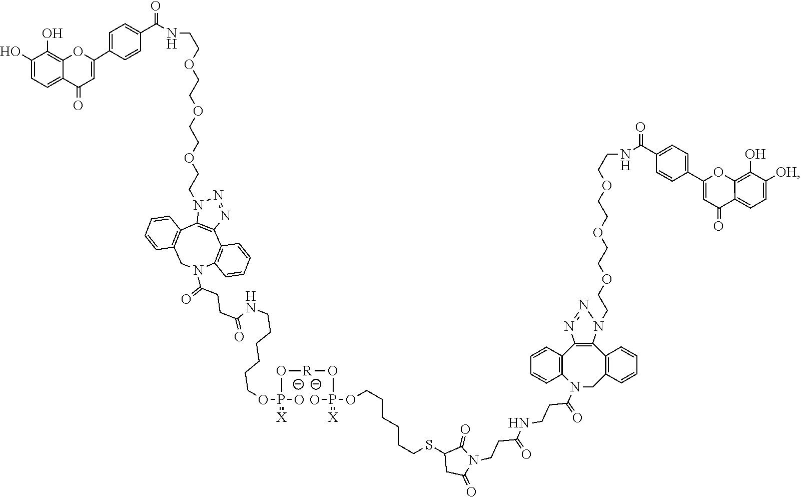

wherein X is S and R is the siRNA targeting Target A (i.e., R is attached to the TrkB ligand at the 5′ and 3′ end of the sense strand).

Compound P was evaluated in an in vivo human Target A transgenic mice PD study carried out as described in Example 41. Results are presented in Table 24 below as percent inhibition of Target A, relative to vehicle control.

TABLE 24
Average Target A Inhibition by Compound P

| Brain Region | Day 15 |
|---|---|
| Cortex | 54% |
| Hippocampus | 69% |
| Cerebellum | 52% |
| Striatum | 56% |
| Cervical Spinal Cord | 68% |

### Example 51: Effect of Compound Q Targeting Target a in Various Brain Regions

Compound Q has the following structure (an siRNA targeting Target A having the following TrkB ligands with Core structures BA-118 and BA-170 attached at the 5′ and 3′ ends of the sense strand, respectively):

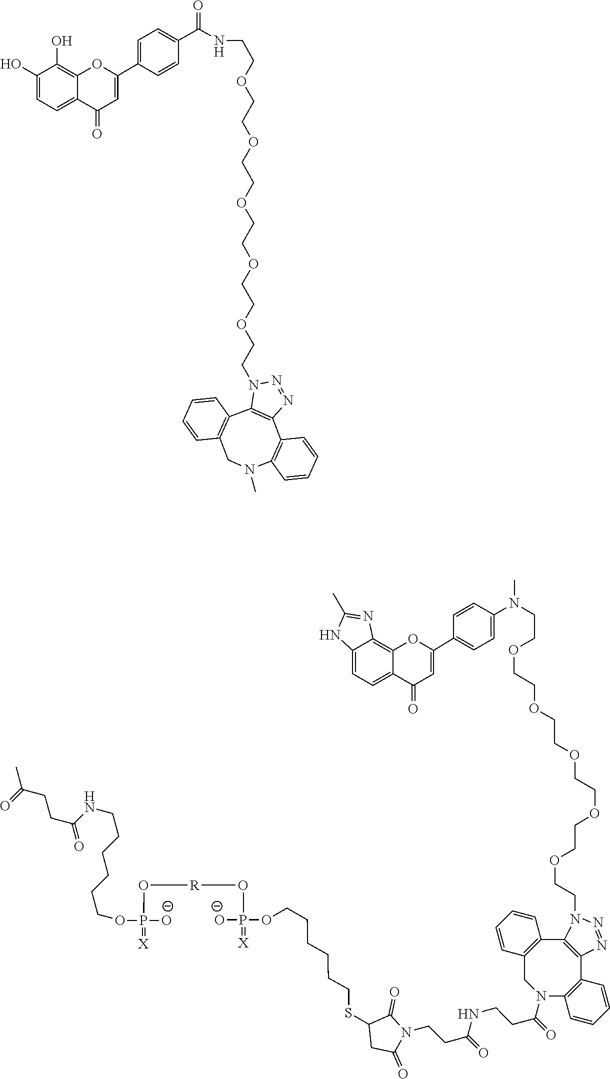

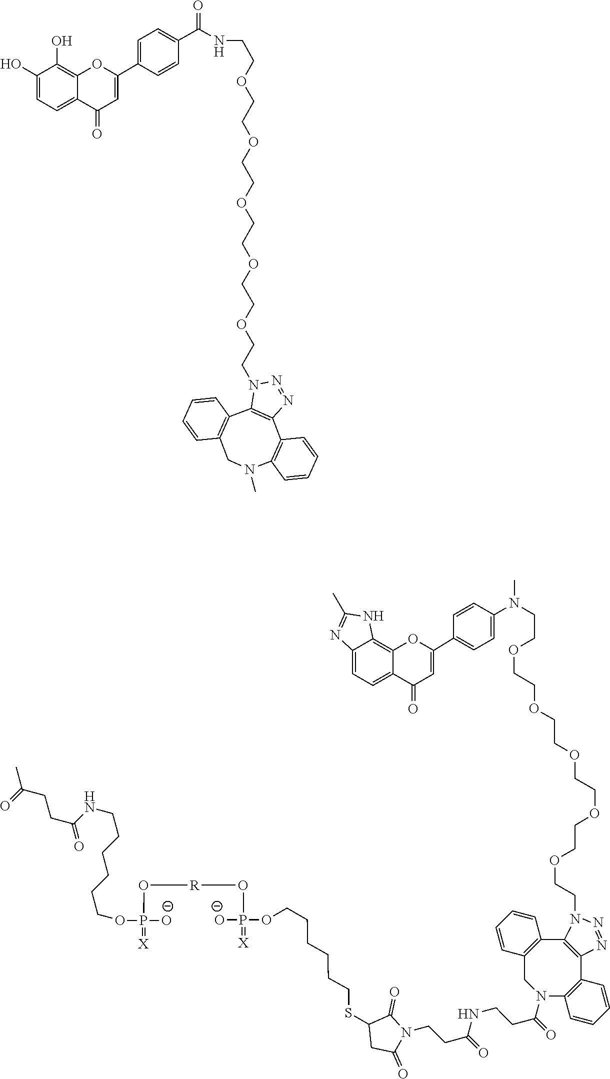

wherein X is S and R is the siRNA targeting Target A (i.e., R is attached to the TrkB ligands with Core structures BA-118 and BA-170 at the 5′ and 3′ ends of the sense strand, respectively). BA-170 is a mixture of both isomers.

Compound Q was evaluated in an in vivo human Target A transgenic mice PD study carried out as described in Example 41. Results are presented in Table 25 below as percent inhibition of Target A, relative to vehicle control.

TABLE 25
Average Target A Inhibition by Compound Q

| Brain Region | Day 15 |
|---|---|
| Cortex | 50% |
| Hippocampus | 66% |
| Cerebellum | 49% |
| Striatum | 63% |
| Cervical Spinal Cord | 69% |

### Example 52: Effect of Compound R Targeting Target a in Various Brain Regions

Compound R has the following structure (an siRNA targeting Target A having a TrkB ligand with Core structure BA-118 attached to the 3′ end of the sense strand):

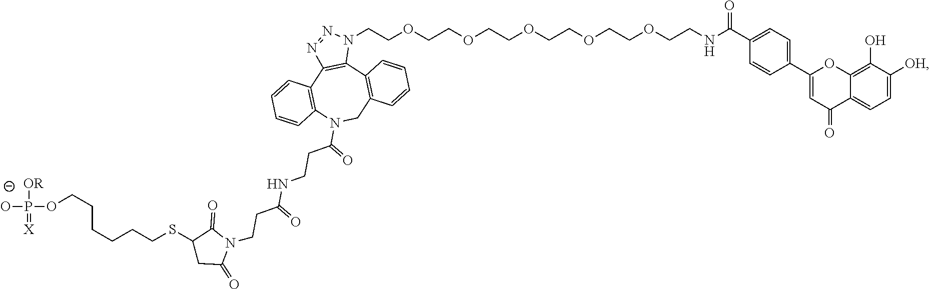

wherein X is S and R is the siRNA targeting Target A (i.e., R is attached to the TrkB ligand at the 3′ end of the sense strand).

Compound R was evaluated in an in vivo human Target A transgenic mice PD study carried out as described in Example 41. Results are presented in Table 26 below as percent inhibition of Target A, relative to vehicle control.

TABLE 26
Average Target A Inhibition by Compound R

| Brain Region | Day 15 |
|---|---|
| Cortex | 45% |
| Hippocampus | 51% |
| Cerebellum | 41% |
| Striatum | 63% |
| Cervical Spinal Cord | 40% |

### Example 53: Effect of Compound S Targeting Target a in Various Brain Regions

Compound S has the following structure (an siRNA targeting Target A having a TrkB ligand with Core structure BA-183 attached to the 5′ end of the sense strand):

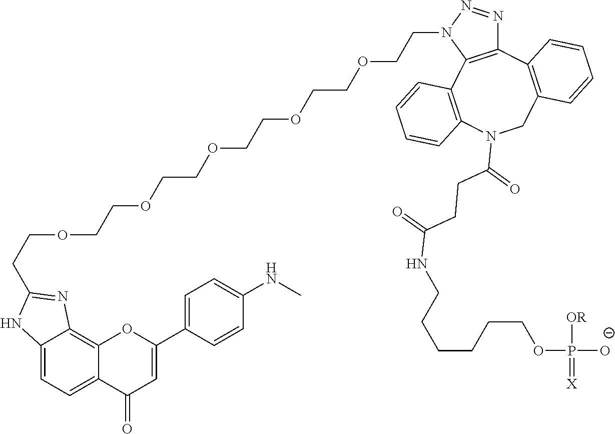

wherein X is S and R is the siRNA targeting Target A (i.e., R is attached to the TrkB ligand at the 5′ end of the sense strand).

Compound S was evaluated in an in vivo human Target A transgenic mice PD study carried out as described in Example 41. Results are presented in Table 27 below as percent inhibition of Target A, relative to vehicle control.

TABLE 27
Average Target A Inhibition by Compound Compound S

| Brain Region | Day 15 |
|---|---|
| Cortex | 44% |
| Hippocampus | 56% |
| Cerebellum | 55% |
| Striatum | 59% |
| Cervical Spinal Cord | 56% |

### INCORPORATION BY REFERENCE

The contents of all references (including literature references, issued patents, published patent applications, and co-pending patent applications) cited throughout this application are hereby expressly incorporated herein in their entireties by reference.

### EQUIVALENTS

Those skilled in the art will recognize or be able to ascertain using no more than routine experimentation, many equivalents of the specific embodiments described herein. Such equivalents are intended to be encompassed by the following claims.

What is claimed is:

1. A compound, or a stereoisomer, tautomer, prodrug, or salt thereof, comprising the structure of Formula (V): 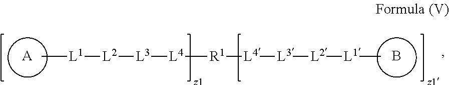
wherein:
each 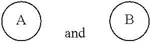
are independently a Tropomyosin receptor B (TrkB) ligand;
each of L¹, L², L³, L⁴, L^{1′}, L^{2′}, L^{3′}, and L^{4′} is independently a linker, bond, or absent;
R¹ is an oligonucleotide, a protecting group, a small molecule, a protein, an antibody, or a peptide;
z1 is 1, 2, or 3; and
z1′ is 0, 1, 2, or 3.

2. The compound of claim 1, or a stereoisomer, tautomer, prodrug, or salt thereof, wherein:
z1 is 1; and
z1′ is 0 or 1.

3. The compound, or a stereoisomer, tautomer, prodrug, or salt thereof, of claim 2, wherein z1 is 1 and z1′ is 0.

4. The compound, or a stereoisomer, tautomer, prodrug, or salt thereof, of any one of claims 1-3, wherein the compound comprises the structure of Formula (I):

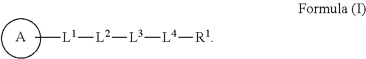

5. The compound, or a stereoisomer, tautomer, prodrug, or salt thereof, of claim 4, comprising the structure of Formula (I′): 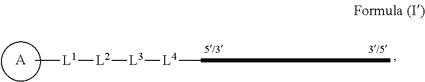
wherein: [symbol]
is an oligonucleotide.

6. The compound, or a stereoisomer, tautomer, prodrug, or salt thereof, of claim 1 or 2, wherein z1 and z1′ are each independently 1.

7. The compound, or a stereoisomer, tautomer, prodrug, or salt thereof, of claim 6, wherein the compound comprises the structure of Formula (VI):

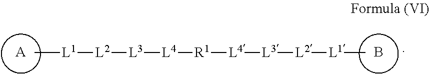

8. The compound, or a stereoisomer, tautomer, prodrug, or salt thereof, of claim 7, wherein the compound comprises the structure of Formula (VI′): 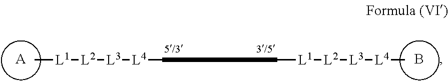
wherein: [symbol]
is an oligonucleotide.

9. The compound, or a stereoisomer, tautomer, prodrug, or salt thereof, of any one of claims 1-8, wherein the TrkB ligand comprises a substituted or unsubstituted aryl, substituted or unsubstituted heteroaryl, substituted or unsubstituted heterocycle, substituted or unsubstituted heterocycloalkyl, or substituted or unsubstituted cycloalkyl.

10. The compound, or a stereoisomer, tautomer, prodrug, or salt thereof, of any one of claims 1-9, wherein the TrkB ligand comprises a substituted or unsubstituted aryl, substituted or unsubstituted heteroaryl, or substituted or unsubstituted heterocycloalkyl.

11. The compound, or a stereoisomer, tautomer, prodrug, or salt thereof, of any one of claims 1-10, wherein the TrkB ligand comprises a substituted or unsubstituted mono- or polycyclic aryl, substituted or unsubstituted mono- or polycyclic heteroaryl, substituted or unsubstituted mono- or polycyclic heterocycloalkyl, or substituted or unsubstituted mono- or polycyclic cycloalkyl.

12. The compound, or a stereoisomer, tautomer, prodrug, or salt thereof, of claim 11, wherein the substituted or unsubstituted polycyclic aryl, substituted or unsubstituted polycyclic heteroaryl, substituted or unsubstituted polycyclic heterocycloalkyl or substituted or unsubstituted mono- or polycyclic cycloalkyl are a fused ring system.

13. The compound, or a stereoisomer, tautomer, prodrug, or salt thereof, of claim 12, wherein the fused ring system comprises two to four fused rings.

14. The compound, or a stereoisomer, tautomer, prodrug, or salt thereof, of claim 13, wherein the fused ring system further comprises an optionally substituted pendant cycloalkyl, optionally substituted pendant heterocycloalkyl, optionally substituted pendant aryl, or an optionally substituted pendant heteroaryl.

15. The compound, or a stereoisomer, tautomer, prodrug, or salt thereof, of any one of claims 1-14, wherein each TrkB ligand is independently: 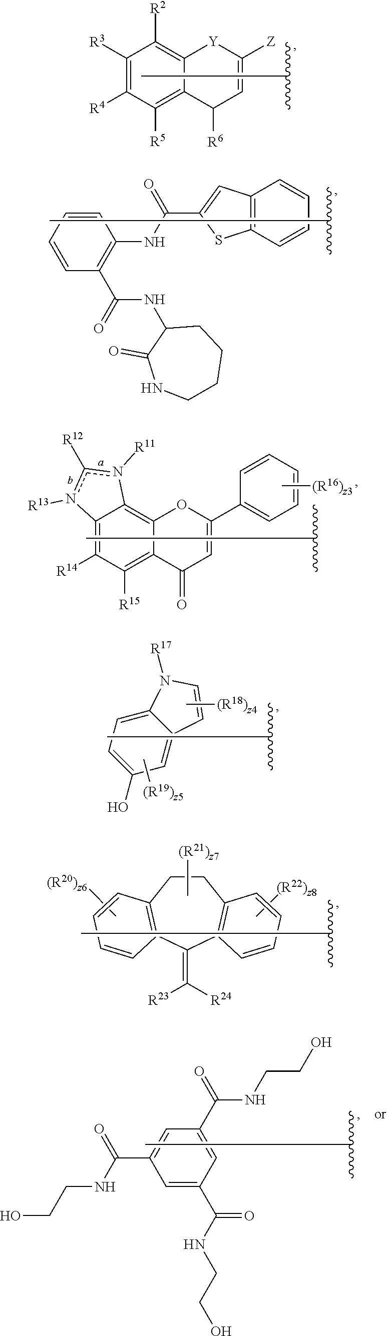 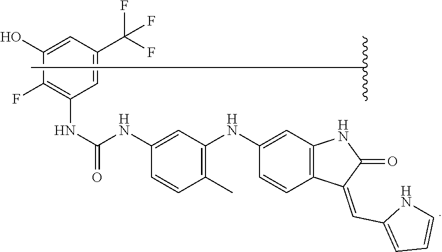
R² is hydrogen, —OR⁷, —SR⁸, or —NR⁹R¹⁰;
R³ is hydrogen, —OR³¹, —SR³², or —NR³³R³⁴;
R⁴ is hydrogen, —OR³⁵, —SR³⁶, or —NR³⁷R³⁸;
R⁵ is hydrogen, —OR³⁹, —SR⁴⁰, or —NR⁴¹R⁴²;
R⁶ is hydrogen, —OH, optionally substituted —O-alkyl, optionally substituted —OAc, —NH₂, optionally substituted —NHAc, —SH, or ═O;
R⁷, R⁸, R⁹, R¹⁰, R³¹, R³², R³³, R³⁴, R³⁵, R³⁶, R³⁷, R³⁸, R³⁹, R⁴⁰, R⁴¹, and R⁴² are each independently hydrogen, optionally substituted alkyl, optionally substituted heteroalkyl, optionally substituted cycloalkyl, optionally substituted heterocycloalkyl, optionally substituted aryl, optionally substituted heteroaryl;
Y is CH₂, NH, S, or O;
Z is optionally substituted aryl or optionally substituted heteroaryl;
R¹¹ and R¹³ are each independently absent, hydrogen, or optionally substituted alkyl;
R¹², R¹⁴, and R¹⁵ are each independently hydrogen, optionally substituted alkyl, optionally substituted heteroalkyl, optionally substituted cycloalkyl, optionally substituted heterocycloalkyl, optionally substituted aryl, or optionally substituted heteroaryl;
R¹⁶ is hydrogen, halogen, —CN, —N₃, —SOₙ₁₆R^{1A}, —SOᵥ₁₆NR^{16B}R^{16C}, —NHNR^{16B}R^{16C}, —ONR^{16B}R^{16C}, —NHC(O)NHNR^{16B}R^{16C}, —NHC(O)NR^{16B}R^{16C}, —N(O)ₘ₁₆, —NR^{16B}R^{16C}, —C(O)R^{16D}, —C(O)OR^{16D}, —C(O)NR^{16B}R^{16C}, —OR^{16A}, —NR^{16B}SO₂R^{16A}, —NR^{16B}C(O)R^{16D};
—NR^{16B}C(O)OR^{16D}, —NR^{16B}OR^{16D}, optionally substituted alkyl, optionally substituted heteroalkyl, optionally substituted cycloalkyl, optionally substituted heterocycloalkyl, optionally substituted aryl, or optionally substituted heteroaryl; [symbol]
and [symbol] are each independently a single bond or a double bond, wherein if [symbol] is a single bond, then [symbol] is a double bond and R¹³ is absent; and further wherein if [symbol] is a single bond, then [symbol] is a double bond and R¹¹ is absent;
R^{16A}, R^{16B}, R^{16C}, R^{16D} are each independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{16B} and R^{16C} substituents bonded to the same nitrogen atom may optionally be joined to form a substituted or unsubstituted heterocycloalkyl or substituted or unsubstituted heteroaryl;
R¹⁷, R¹⁸, and R¹⁹ are each independently hydrogen, optionally substituted alkyl, optionally substituted heteroalkyl, optionally substituted cycloalkyl, optionally substituted heterocycloalkyl, optionally substituted aryl, or optionally substituted heteroaryl;
R²⁰ is hydrogen, halogen, —CN, —N₃, —SOₙ₂₀R^{1A}, —SOᵥ₂₀NR^{20B}R^{20C}, —NHNR^{20B}R^{20C}, —ONR^{20B}R^{20C}, —NHC(O)NHNR^{20B}R^{20C}, —NHC(O)NR^{20B}R^{20C}, —N(O)ₘ₂₀, —NR^{20B}R^{20C}, —C(O)R^{20D}, —C(O)OR^{20D}, —C(O)NR^{20B}R^{20C}, —OR^{20A}, —NR^{20B}SO₂R^{20A}, —NR^{20B}C(O)R^{20D};
—NR^{20B}C(O)OR^{20D}, —NR^{20B}OR^{20D}, optionally substituted alkyl, optionally substituted heteroalkyl, optionally substituted cycloalkyl, optionally substituted heterocycloalkyl, optionally substituted aryl, or optionally substituted heteroaryl;
R²¹ is hydrogen, halogen, —CN, —N₃, —SOₙ₂₁R^{1A}, —SOᵥ₂₁NR^{21B}R^{21C}, —NHNR^{21B}R^{21C}, —ONR^{21B}R^{21C}, —NHC(O)NHNR^{21B}R^{21C}, —NHC(O)NR^{21B}R^{21C}, —N(O)ₘ₂₁, —NR^{21B}R^{21C}, —C(O)R^{21D}, —C(O)OR^{21D}, —C(O)NR^{21B}R^{21C}, —OR^{21A}, —NR^{21B}SO₂R^{21A}, —NR^{21B}C(O)R^{21D};
—NR^{21B}C(O)OR^{21D}, —NR^{21B}OR^{21D}, optionally substituted alkyl, optionally substituted heteroalkyl, optionally substituted cycloalkyl, optionally substituted heterocycloalkyl, optionally substituted aryl, or optionally substituted heteroaryl;
R²² and R²³ are each independently hydrogen, optionally substituted alkyl, optionally substituted heteroalkyl, optionally substituted cycloalkyl, optionally substituted heterocycloalkyl, optionally substituted aryl, or optionally substituted heteroaryl;
R²⁴ is hydrogen, halogen, —CN, —N₃, —SOₙ₂₄R^{1A}, —SOᵥ₂₄NR^{24B}R^{24C}, —NHNR^{24B}R^{24C}, —ONR^{24B}R^{24C}, —NHC(O)NHNR^{24B}R^{24C}, —NHC(O)NR^{24B}R^{24C}, —N(O)ₘ₂₄, —NR^{24B}R^{24C}, —C(O)R^{24D}, —C(O)OR^{24D}, —C(O)NR^{24B}R^{24C}, —OR^{24A}, —NR^{24B}SO₂R^{24A}, —NR^{24B}C(O))R^{24D};
—NR^{24B}C(O)OR^{24D}, —NR^{24B}OR^{24D}, optionally substituted alkyl, optionally substituted heteroalkyl, optionally substituted cycloalkyl, optionally substituted heterocycloalkyl, optionally substituted aryl, or optionally substituted heteroaryl;
R^{20A}, R^{20B}, R^{20C}, R^{20D}, R^{21A}, R^{21B}, R^{21C}, R^{21D}, R^{24A}, R^{24B}, R^{24C}, and R^{24D} are each independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{20B}, R^{20C}, R^{21B}, R^{21C}, R^{24B}, R^{24C}, R^{24B}, and R^{24C} substituents bonded to the same nitrogen atom may optionally be joined to form a substituted or unsubstituted heterocycloalkyl or substituted or unsubstituted heteroaryl;
n16, n20, n21, n23, n24, z6, and z8 are each independently 0, 1, 2, 3, or 4;
v16, v20, v21, m16, m20, m21, and m24 are each independently 1 or 2;
z3 is 0, 1, 2, 3, 4, or 5;
z4 and z7 are each independently 0, 1, or 2;
z5 is 0, 1, 2, or 3; and
z6 and z8 are each independently 0, 1, 2, 3, or 4.

16. The compound, or a stereoisomer, tautomer, prodrug, or salt thereof, of any one of claims 1-15, wherein each TrkB ligand is independently flavone, tropoflavin, or derivatives thereof.

17. The compound, or a stereoisomer, tautomer, prodrug, or salt thereof, of any one of claims 1-16, wherein each TrkB ligand is independently 3,7-dihydroxyflavone, 3,7,8,2′-tetrahydroxyflavone, 7,3′-dihydroxyflavone, 7,8,2′-trihydroxyflavone, 7,8,3′-trihydroxyflavone, 7,8,4′-trihydroxyflavone, diosmetin (5,7,3′-trihydroxy-4′-methoxyflavone), 7-hydroxy-4′-methoxyflavone, 8-hydroxy-7-methoxyflavone, eutropoflavin (4′-dimethylamino-7,8-dihydroxyflavone), norwogonin (5,7,8-trihydroxyflavone), R7, R13, tropoflavin (7,8-dihydroxyflavone), quercetin (3,3′,4′,5,7-pentahydroxyflavone), apigenin (4′,5,7-trihydroxyflavone), isocoumarin, gossypetin (3,5,7,8,3′,4′-hexahydroxyflavone), 2-methyl-8-phenylchromeno[7,8-d]imidazol-6 (3H)-one, 8-phenylchromeno[7,8-d]imidazol-6 (3H)-one, 4-oxo-2-phenyl-4H-chromene-7,8-diyl diacetate, ANA-12, an anti-TrkB antibody, or derivatives thereof.

18. A compound, or a stereoisomer, tautomer, prodrug, or salt thereof, comprising the structure of Formula (II): 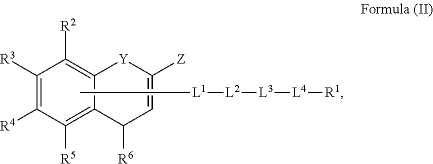
wherein:
R² is hydrogen, —OR⁷, —SR⁸, or —NR⁹R¹⁰;
R³ is hydrogen, —OR³¹, —SR³², or —NR³³R³⁴;
R⁴ is hydrogen, —OR³⁵, —SR³⁶, or —NR³⁷R³⁸;
R⁵ is hydrogen, —OR³⁹, —SR⁴⁰, or —NR⁴¹R⁴²;
R⁶ is hydrogen, —OH, optionally substituted —O-alkyl, optionally substituted —OAc, —NH₂, optionally substituted —NHAc, —SH, or ═O;
R⁷, R⁸, R⁹, R¹⁰, R³¹, R³², R³³, R³⁴, R³⁵, R³⁶, R³⁷, R³⁸, R³⁹, R⁴⁰, R⁴¹, and R⁴² are each independently hydrogen, optionally substituted alkyl, optionally substituted heteroalkyl, optionally substituted cycloalkyl, optionally substituted heterocycloalkyl, optionally substituted aryl, optionally substituted heteroaryl;
Y is CH₂, NH, S, or O; and
Z is optionally substituted aryl or optionally substituted heteroaryl.

19. The compound, or a stereoisomer, tautomer, prodrug, or salt thereof, of claim 18, wherein the compound comprises the structure of Formula (II-a):

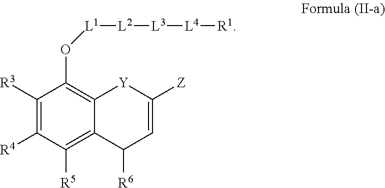

20. The compound, or a stereoisomer, tautomer, prodrug, or salt thereof, of claim 18, wherein the compound comprises the structure of Formula (II-b):

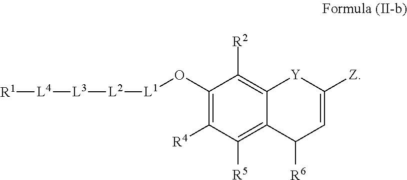

21. The compound, or a stereoisomer, tautomer, prodrug, or salt thereof, of claim 18, wherein the compound comprises the structure of Formula (II-c):

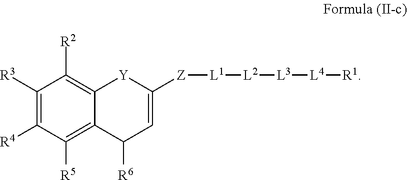

22. The compound, or a stereoisomer, tautomer, prodrug, or salt thereof, of any one of claims 15-21, wherein R² is —OH.

23. The compound, or a stereoisomer, tautomer, prodrug, or salt thereof, of any one of claims 15-22, wherein R³ is —OH.

24. The compound, or a stereoisomer, tautomer, prodrug, or salt thereof, of any one of claims 15-23, wherein R⁴ is hydrogen.

25. The compound, or a stereoisomer, tautomer, prodrug, or salt thereof, of any one of claims 15-24, wherein R⁵ is hydrogen.

26. The compound, or a stereoisomer, tautomer, prodrug, or salt thereof, of any one of claims 15-25, wherein R⁶ is ═O.

27. The compound, or a stereoisomer, tautomer, prodrug, or salt thereof, of any one of claims 15-26, wherein Y is O.

28. The compound, or a stereoisomer, tautomer, prodrug, or salt thereof, of any one of claims 15-27, wherein Z is optionally substituted aryl.

29. The compound, or a stereoisomer, tautomer, prodrug, or salt thereof, of any one of claims 15-28, wherein Z is optionally substituted phenyl.

30. The compound, or a stereoisomer, tautomer, prodrug, or salt thereof, of any one of claims 15-29, wherein Z is unsubstituted phenyl.

31. The compound, or a stereoisomer, tautomer, prodrug, or salt thereof, of any one of claims 15-29, wherein Z is phenyl substituted with one or more —OH and/or —O-alkyl.

32. A compound, or a stereoisomer, tautomer, prodrug, or salt thereof, wherein the compound comprises the structure of Formula (III): 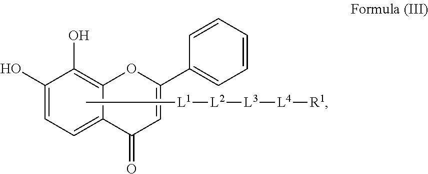
wherein each of L¹, L², L³, and L⁴ is independently a linker, bond, or absent; and
R¹ is an oligonucleotide, a protecting group, a small molecule, a protein, an antibody, or a peptide.

33. The compound, or a stereoisomer, tautomer, prodrug, or salt thereof, of claim 32, wherein the compound comprises the structure of Formula (III-a):

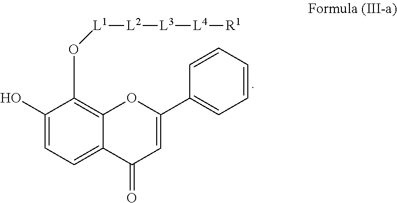

34. The compound, or a stereoisomer, tautomer, prodrug, or salt thereof, of claim 32, wherein the compound comprises the structure of Formula (III-b):

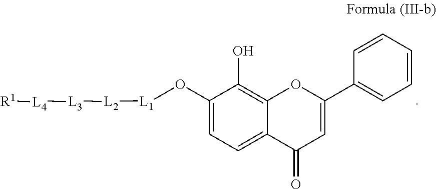

35. The compound, or a stereoisomer, tautomer, prodrug, or salt thereof, of claim 32, wherein the compound comprises the structure of Formula (III-c):

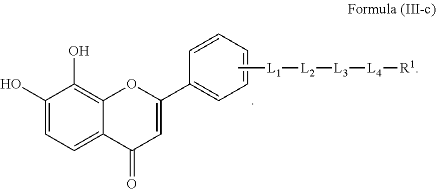

36. The compound, or a stereoisomer, tautomer, prodrug, or salt thereof, of claim 35, wherein the compound comprises the structure of Formula (III-c-1):

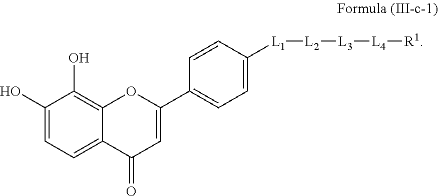

37. The compound, or a stereoisomer, tautomer, prodrug, or salt thereof, of any one of claims 1-36, wherein the TrkB ligand is ANA-12, or a prodrug or derivative thereof.

38. A compound, or a stereoisomer, tautomer, prodrug, or salt thereof, wherein the compound comprises the structure of Formula (II′): 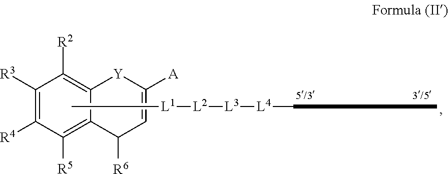
wherein:
each of L¹, L², L³, and L⁴ is independently a linker, bond, or absent;
R² is hydrogen, —OR⁷, —SR⁸, or —NR⁹R¹⁰;
R³ is hydrogen, —OR³¹, —SR³², or —NR³³R³⁴;
R⁴ is hydrogen, —OR³⁵, —SR³⁶, or —NR³⁷R³⁸;
R⁵ is hydrogen, —OR³⁹, —SR⁴⁰, or —NR⁴¹R⁴²;
R⁶ is hydrogen, —OH, optionally substituted —O-alkyl, optionally substituted —OAc, —NH₂, optionally substituted —NHAc, —SH, or ═O;
R⁷, R⁸, R⁹, R¹⁰, R³¹, R³², R³³, R³⁴, R³⁵, R³⁶, R³⁷, R³⁸, R³⁹, R⁴⁰, R⁴¹, and R⁴² are each independently hydrogen, optionally substituted alkyl, optionally substituted heteroalkyl, optionally substituted cycloalkyl, optionally substituted heterocycloalkyl, optionally substituted aryl, optionally substituted heteroaryl;
Y is CH₂, NH, S, or O; and
Z is optionally substituted aryl or optionally substituted heteroaryl; and [symbol]
is an oligonucleotide.

39. A compound, or a stereoisomer, tautomer, prodrug, or salt thereof, wherein the compound comprises the structure of Formula (II″): 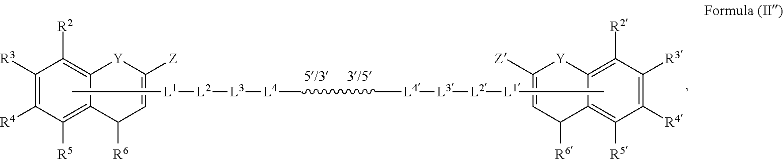
wherein: [symbol]
is an oligonucleotide;
each of L¹, L², L³, L⁴, L¹, L², L^{3′}, and L^{4′} is independently a linker, bond, or absent;
R² is hydrogen, —OR⁷, —SR⁸, or —NR⁹R¹⁰;
R^{2′} is hydrogen, —OR^{7′}, —SR^{8′}, or —NR^{9′}R^{10′};
R³ is hydrogen, —OR³¹, —SR³², or —NR³³R³⁴;
R^{3′} is hydrogen, —OR^{31′}, —SR^{32′}, or —NR^{33′}R^{34′};
R⁴ is hydrogen, —OR³⁵, —SR³⁶, or —NR³⁷R³⁸;
R^{4′} is hydrogen, —OR^{35′}, —SR^{36′}, or —NR^{37′}R^{38′};
R⁵ is hydrogen, —OR³⁹, —SR⁴⁰, or —NR⁴¹R⁴²;
R^{5′} is hydrogen, —OR^{39′}, —SR^{40′}, or —NR^{41′}R^{42′};
R⁶ is hydrogen, —OH, optionally substituted —O-alkyl, optionally substituted —OAc, —NH₂, optionally substituted —NHAc, —SH, or ═O;
R^{6′} is hydrogen, —OH, optionally substituted —O-alkyl, optionally substituted —OAc, —NH₂, optionally substituted —NHAc, —SH, or ═O;
R⁷, R^{7′}, R⁸, R^{8′}, R⁹, R^{9′}, R¹⁰, R^{10′}, R³¹, R^{31′}, R³², R^{32′}, R³³, R^{33′}, R³⁴, R^{34′}, R³⁵, R^{35′}, R³⁶, R^{36′}, R³⁷, R^{37′}, R³⁸, R^{38′}, R³⁹, R^{39′}, R⁴⁰, R^{40′}, R⁴¹, R^{41′}, R⁴², and R^{42′} are each independently hydrogen, optionally substituted alkyl, optionally substituted heteroalkyl, optionally substituted cycloalkyl, optionally substituted heterocycloalkyl, optionally substituted aryl, optionally substituted heteroaryl;
Y and Y′ are each independently CH₂, NH, S, or O; and
Z and Z′ are each independently optionally substituted aryl or optionally substituted heteroaryl.

40. A compound, or a stereoisomer, tautomer, prodrug, or salt thereof, comprising the structure of Formula (IV): 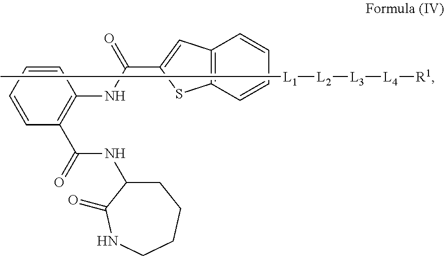
wherein each of L¹, L², L³, and L⁴ is independently a linker, bond, or absent; and
R¹ is an oligonucleotide, a protecting group, a small molecule, a protein, an antibody, or a peptide.

41. The compound, or a stereoisomer, tautomer, prodrug, or salt thereof, of claim 40, wherein the compound comprises the structure of Formula (IV-a):

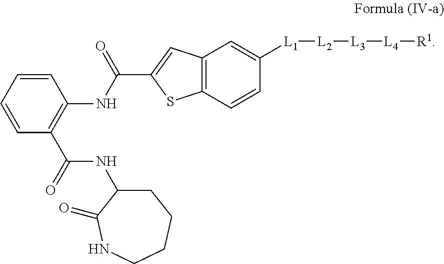

42. The compound, or a stereoisomer, tautomer, prodrug, or salt thereof, of claim 40, wherein the compound comprises the structure of Formula (IV-b):

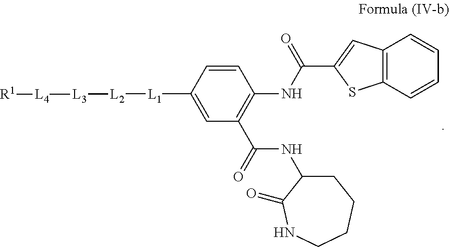

43. A compound, or a stereoisomer, tautomer, prodrug, or salt thereof, comprising the structure of Formula (VII): 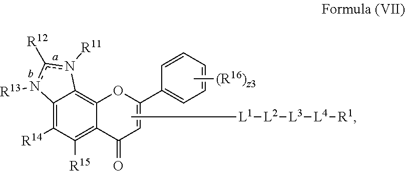
wherein:
each of L¹, L², L³, and L⁴ is independently a linker, bond, or absent;
R¹ is an oligonucleotide, a protecting group, a small molecule, a protein, an antibody, or a peptide;
R¹¹ and R¹³ are each independently absent, hydrogen, or optionally substituted alkyl;
R¹², R¹⁴, and R¹⁵ are each independently hydrogen, optionally substituted alkyl, optionally substituted heteroalkyl, optionally substituted cycloalkyl, optionally substituted heterocycloalkyl, optionally substituted aryl, or optionally substituted heteroaryl;
R¹⁶ is hydrogen, halogen, —CN, —N₃, —SOₙ₁₆R^{1A}, —SOᵥ₁₆NR^{16B}R^{16C}, —NHNR^{16B}R^{16C}, —ONR^{16B}R^{16C}, —NHC(O)NHNR^{16B}R^{16C}, —NHC(O)NR^{16B}R^{16C}, —N(O)ₘ₁₆, —NR^{16B}R^{16C}, —C(O)R^{16D}, —C(O)OR^{16D}, —C(O)NR^{16B}R^{16C}, —OR^{16A}, —NR^{16B}SO₂R^{16A}, —NR^{16B}C(O)R^{16D};
—NR^{16B}C(O)OR^{16D}, —NR^{16B}OR^{16D}, optionally substituted alkyl, optionally substituted heteroalkyl, optionally substituted cycloalkyl, optionally substituted heterocycloalkyl, optionally substituted aryl, or optionally substituted heteroaryl; [symbol]
and [symbol] are each independently a single bond or a double bond, wherein if [symbol] is a single bond, then [symbol] is a double bond and R¹³ is absent; and further wherein if [symbol] is a single bond, then [symbol] is a double bond and R¹¹ is absent;
R^{16A}, R^{16B}, R^{16C}, R^{16D} are each independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{16B} and R^{16C} substituents bonded to the same nitrogen atom may optionally be joined to form a substituted or unsubstituted heterocycloalkyl or substituted or unsubstituted heteroaryl;
z3 is 0, 1, 2, 3, 4, or 5;
n16 is 0, 1, 2, 3, or 4; and
v16 and m16 are each independently 1 or 2.

44. The compound, or a stereoisomer, tautomer, prodrug, or salt thereof, of claim 43, wherein the compound comprises the structure of Formula (VII-a), Formula (VII-b), or a mixture thereof:

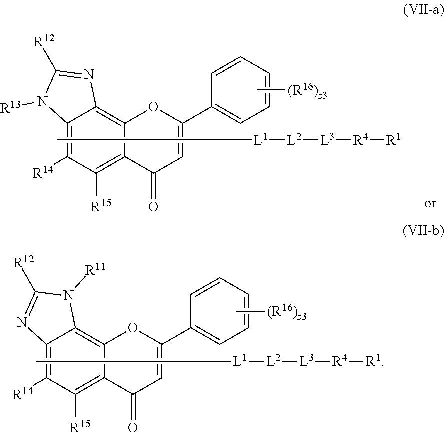

45. The compound, or a stereoisomer, tautomer, prodrug, or salt thereof, of claim 44, wherein the compound comprises the structure of Formula (VII-c):

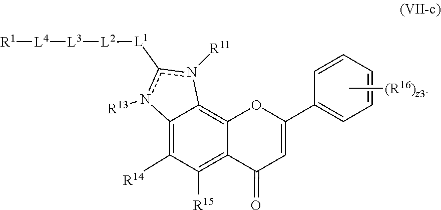

46. The compound, or a stereoisomer, tautomer, prodrug, or salt thereof, of claim 44 or 45, wherein the compound comprises the structure of Formula (VII-c-1), Formula (VII-c-2), or a mixture thereof:

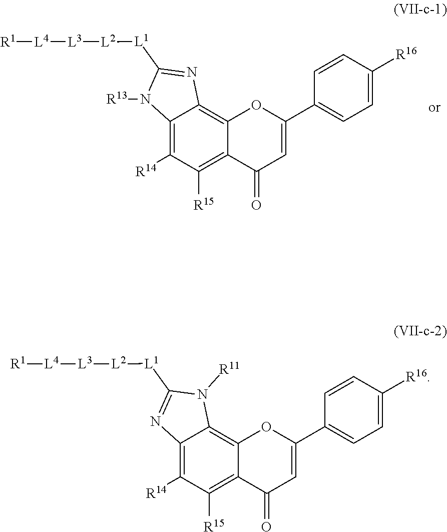

47. The compound, or a stereoisomer, tautomer, prodrug, or salt thereof, of claim 46, wherein R¹⁶ is hydrogen, optionally substituted alkyl, or optionally substituted heteroalkyl.

48. The compound, or a stereoisomer, tautomer, prodrug, or salt thereof, of claim 46 or 47, wherein:
R¹⁶ is —NR^{16B}R^{16C}; and
R^{16B} and R^{16C} are each independently hydrogen or optionally substituted alkyl.

49. The compound, or a stereoisomer, tautomer, prodrug, or salt thereof, of claim 48, wherein:
R^{16B} is hydrogen; and
R^{16C} is C₁-C₃ alkyl.

50. The compound, or a stereoisomer, tautomer, prodrug, or salt thereof, of claim 49, wherein R^{16C} is —CH₃.

51. The compound, or a stereoisomer, tautomer, prodrug, or salt thereof, of claim 43, wherein the compound comprises the structure of Formula (VII-d):

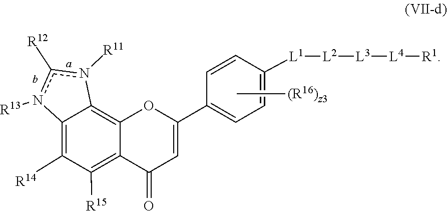

52. The compound, or a stereoisomer, tautomer, prodrug, or salt thereof, of claim 43 or 51, wherein the compound comprises the structure of Formula (VII-d-1), Formula (VII-d-2), or a mixture thereof:

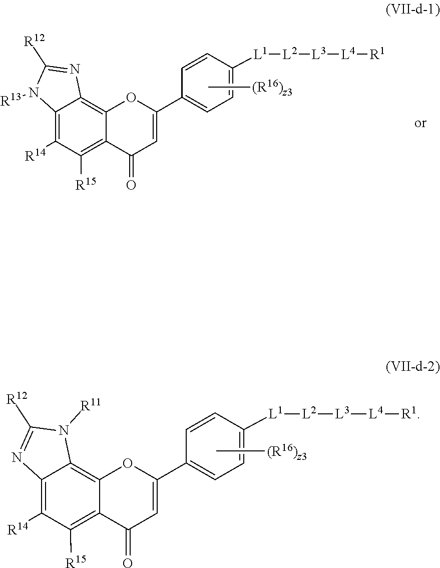

53. The compound, or a stereoisomer, tautomer, prodrug, or salt thereof, of claim 52, wherein R¹² is hydrogen or optionally substituted alkyl.

54. The compound, or a stereoisomer, tautomer, prodrug, or salt thereof, of claim 53, wherein R¹² is hydrogen or C₁-C₃ alkyl.

55. The compound, or a stereoisomer, tautomer, prodrug, or salt thereof, of claim 54, wherein R¹² is hydrogen or —CH₃.

56. A compound, or a stereoisomer, tautomer, prodrug, or salt thereof, wherein the compound comprises the structure of Formula (II″): 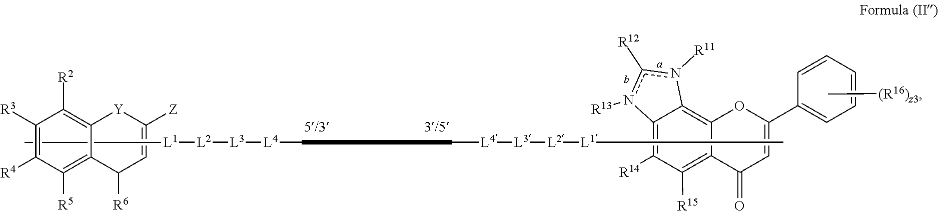
wherein: [symbol]
is an oligonucleotide;
each of L¹, L², L³, L⁴, L^{1′}, L^{2′}, L^{3′}, and L^{4′} is independently a linker, bond, or absent;
R² is hydrogen, —OR⁷, —SR⁸, or —NR⁹R¹⁰;
R³ is hydrogen, —OR³¹, —SR³², or —NR³³R³⁴;
R⁴ is hydrogen, —OR³⁵, —SR³⁶, or —NR³⁷R³⁸;
R⁵ is hydrogen, —OR³⁹, —SR⁴⁰, or —NR⁴¹R⁴²;
R⁶ is hydrogen, —OH, optionally substituted —O-alkyl, optionally substituted —OAc, —NH₂, optionally substituted —NHAc, —SH, or ═O;
R⁷, R⁸, R⁹, R¹⁰, R³¹, R³², R³³, R³⁴, R³⁵, R³⁶, R³⁷, R³⁸, R³⁹, R⁴⁰, R⁴¹, and R⁴² are each independently hydrogen, optionally substituted alkyl, optionally substituted heteroalkyl, optionally substituted cycloalkyl, optionally substituted heterocycloalkyl, optionally substituted aryl, optionally substituted heteroaryl;
Y is CH₂, NH, S, or O;
Z is optionally substituted aryl or optionally substituted heteroaryl;
R¹¹ and R¹³ are each independently absent, hydrogen, or optionally substituted alkyl;
R¹², R¹⁴, R¹⁵, and R¹⁶ are each independently hydrogen, optionally substituted alkyl, optionally substituted heteroalkyl, optionally substituted cycloalkyl, optionally substituted heterocycloalkyl, optionally substituted aryl, optionally substituted heteroaryl; [symbol]
and [symbol] are each independently a single bond or a double bond, wherein if [symbol] is a single bond, then [symbol] is a double bond and R¹³ is absent; and further wherein if [symbol] is a single bond, then [symbol] is a double bond and R¹¹ is absent; and
z3 is 0, 1, 2, 3, 4, or 5.

57. A compound, or a stereoisomer, tautomer, prodrug, or salt thereof, wherein the compound comprises the structure of Formula (II″): 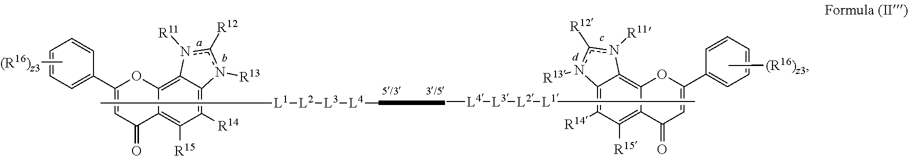
wherein: [symbol]
is an oligonucleotide;
each of L¹, L², L³, L⁴, L^{1′}, L^{2′}, L^{3′}, and L^{4′} is independently a linker, bond, or absent;
R¹¹, R^{11′}, R¹³, and R^{13′} are each independently absent, hydrogen, or optionally substituted alkyl;
R¹², R^{12′}, R¹⁴, R^{14′}, R¹⁵, R^{15′}, R¹⁶, and R^{16′} are each independently hydrogen, optionally substituted alkyl, optionally substituted heteroalkyl, optionally substituted cycloalkyl, optionally substituted heterocycloalkyl, optionally substituted aryl, optionally substituted heteroaryl;
z3 and z3′ are each independently 0, 1, 2, 3, or 5; [symbol]
and [symbol] are each independently a single bond or a double bond, wherein if [symbol] is a single bond, then [symbol] is a double bond and R¹³ is absent; and further wherein if [symbol] is a single bond, then [symbol] is a double bond and R¹¹ is absent; and [symbol]
and [symbol] are each independently a single bond or a double bond, wherein it 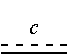
is a single bond, then 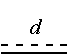
is a double bond and R^{13′} is absent; and further wherein it 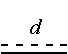
is a single bond, then 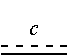
is a double bond and R^{11′} is absent.

58. A compound, or a stereoisomer, tautomer, prodrug, or salt thereof, comprising the structure of Formula (VIII): 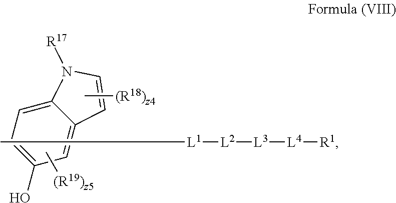
wherein:
each of L¹, L², L³, and L⁴ is independently a linker, bond, or absent;
R¹⁷, R¹⁸, and R¹⁹ are each independently hydrogen, optionally substituted alkyl, optionally substituted heteroalkyl, optionally substituted cycloalkyl, optionally substituted heterocycloalkyl, optionally substituted aryl, or optionally substituted heteroaryl;
z4 is 0, 1, or 2; and
z5 is 0, 1, 2, or 3.

59. The compound, or a stereoisomer, tautomer, prodrug, or salt thereof, of claim 58, wherein the compound comprises the structure of Formula (VIII-a): 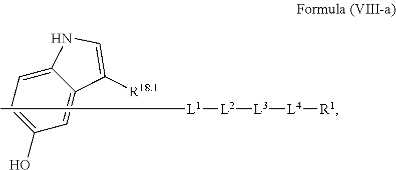
wherein:
R^{18.1} is hydrogen, optionally substituted alkyl or optionally substituted heteroalkyl.

60. The compound, or a stereoisomer, tautomer, prodrug, or salt thereof, of claim 58 or 59, wherein the compound comprises the structure of Formula (VIII-a-1):

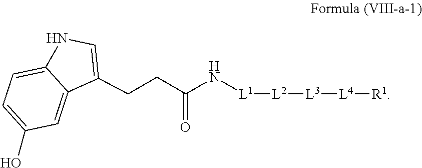

61. A compound, or a stereoisomer, tautomer, prodrug, or salt thereof, comprising the structure of Formula (IX): 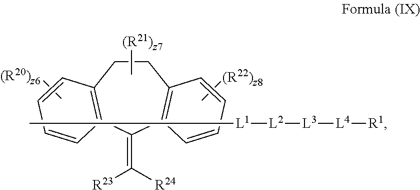
wherein:
each of L¹, L², L³, and L⁴ is independently a linker, bond, or absent;
R²⁰ is hydrogen, halogen, —CN, —N₃, —SOₙ₂₀R^{1A}, —SOᵥ₂₀NR^{20B}R^{20C}, —NHNR^{20B}R^{20C}, —ONR^{20B}R^{20C}, —NHC(O)NHNR^{20B}R^{20C}, —NHC(O)NR^{20B}R^{20C}, —N(O)ₘ₂₀, —NR^{20B}R^{20C}, —C(O)R^{20D}, —C(O)OR^{20D}, —C(O)NR^{20B}R^{20C}, —OR^{20A}, —NR^{20B}SO₂R^{20A}, —NR^{20B}C(O)R^{20D};
—NR^{20B}C(O)OR^{20D}, —NR^{20B}OR^{20D}, optionally substituted alkyl, optionally substituted heteroalkyl, optionally substituted cycloalkyl, optionally substituted heterocycloalkyl, optionally substituted aryl, or optionally substituted heteroaryl;
R²¹ is hydrogen, halogen, —CN, —N₃, —SOₙ₂₁R^{1A}, —SOᵥ₂₁NR^{21B}R^{21C}, —NHNR^{21B}R^{21C}, —ONR^{21B}R^{21C}, —NHC(O)NHNR^{21B}R^{21C}, —NHC(O)NR^{21B}R^{21C}, —N(O)ₘ₂₁, —NR^{21B}R^{21C}, —C(O)R^{21D}, —C(O)OR^{21D}, —C(O)NR^{21B}R^{21C}, —OR^{21A}, —NR^{21B}SO₂R^{21A}, —NR^{21B}C(O)R^{21D};
—NR^{21B}C(O)OR^{21D}, —NR^{21B}OR^{21D}, optionally substituted alkyl, optionally substituted heteroalkyl, optionally substituted cycloalkyl, optionally substituted heterocycloalkyl, optionally substituted aryl, or optionally substituted heteroaryl;
R²² and R²³ are each independently hydrogen, optionally substituted alkyl, optionally substituted heteroalkyl, optionally substituted cycloalkyl, optionally substituted heterocycloalkyl, optionally substituted aryl, or optionally substituted heteroaryl;
R²⁴ is hydrogen, halogen, —CN, —N₃, —SOₙ₂₄R^{24A}, —SOᵥ₂₄NR^{24B}R^{24C}, —NHNR^{24B}R^{24C}, —ONR^{24B}R^{24C}, —NHC(O)NHNR^{24B}R^{24C}, —NHC(O)NR^{24B}R^{24C}, —N(O)ₘ₂₄, —NR^{24B}R^{24C}, —C(O)R^{24D}, —C(O)OR^{24D}, —C(O)NR^{24B}R^{24C}, —OR^{24A}, —NR^{24B}SO₂R^{24A}, —NR^{24B}C(O)R^{24D};
—NR^{24B}C(O)OR^{24D}, —NR^{24B}OR^{24D}, optionally substituted alkyl, optionally substituted heteroalkyl, optionally substituted cycloalkyl, optionally substituted heterocycloalkyl, optionally substituted aryl, or optionally substituted heteroaryl;
R^{20A}, R^{20B}, R^{20C}, R^{20D}, R^{21A}, R^{21B}, R^{21C}, R^{21D}, R^{24A}, R^{24B}, R^{24C}, and R^{24D} are each independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{20B}, R^{20C}, R^{21B}, R^{21C}, R^{24B}, R^{24C}, R^{24B}, and R^{24C} substituents bonded to the same nitrogen atom may optionally be joined to form a substituted or unsubstituted heterocycloalkyl or substituted or unsubstituted heteroaryl;
n21, n22, n24, z6 and z8 are each independently 0, 1, 2, 3, or 4;
v20, v21, v24, m20, m21, and m24 are each independently 1 or 2; and
z7 is 0, 1, or 2.

62. The compound, or a stereoisomer, tautomer, prodrug, or salt thereof, of claim 61, wherein the compound comprises the structure of Formula (IX-a):

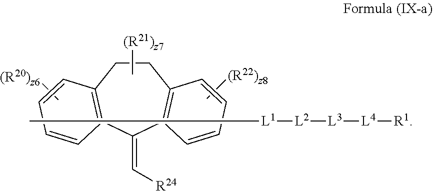

63. The compound, or a stereoisomer, tautomer, prodrug, or salt thereof, of claim 61 or 62, wherein the compound comprises the structure of Formula (IX-a-1): 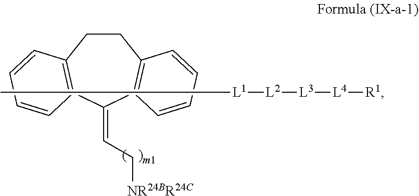
wherein:
R^{24B} and R^{24C} are each independently optionally substituted alkyl or optionally substituted heteroalkyl; and
m1 is 1, 2, or 3.

64. The compound, or a stereoisomer, tautomer, prodrug, or salt thereof, of any one of claims 61-63, wherein the compound comprises the structure of Formula (IX-a-2):

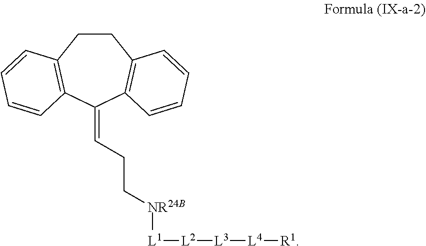

65. The compound, or a stereoisomer, tautomer, prodrug, or salt thereof, of claim 64, wherein R^{24B} is hydrogen or optionally substituted alkyl.

66. The compound, or a stereoisomer, tautomer, prodrug, or salt thereof, of claim 65, wherein R^{24B} is C₁-C₃ alkyl.

67. The compound, or a stereoisomer, tautomer, prodrug, or salt thereof, of claim 66, wherein R^{24B} is —CH₃.

68. A compound, or a stereoisomer, tautomer, prodrug, or salt thereof, comprising the structure of Formula (X): 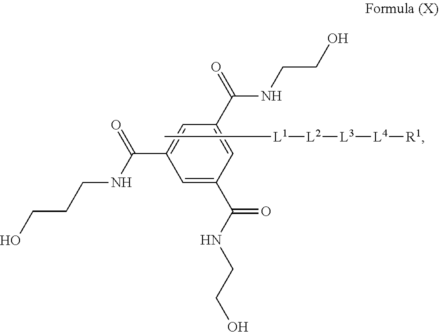
wherein each of L¹, L², L³, and L⁴ is independently a linker, bond, or absent; and
R¹ is an oligonucleotide, a protecting group, a small molecule, a protein, an antibody, or a peptide.

69. The compound, or a stereoisomer, tautomer, prodrug, or salt thereof, of claim 70, wherein the compound comprises the structure of Formula (X-a): 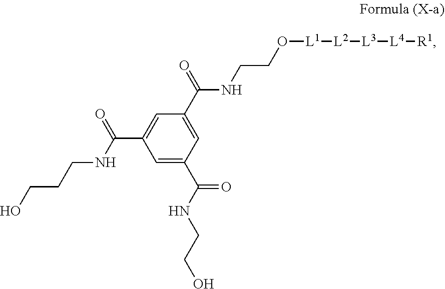
wherein L¹, L², L³, L⁴ is independently a linker, bond, or absent; and
R¹ is an oligonucleotide, a protecting group, a small molecule, a protein, an antibody, or a peptide.

70. A compound, or a stereoisomer, tautomer, prodrug, or salt thereof, comprising the structure of Formula (XI): 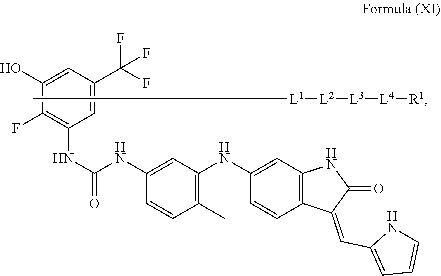
wherein each of L¹, L², L³, and L⁴ is independently a linker, bond, or absent; and
R¹ is an oligonucleotide, a protecting group, a small molecule, a protein, an antibody, or a peptide.

71. The compound, or a stereoisomer, tautomer, prodrug, or salt thereof, of claim 70, wherein the compound comprises the structure of Formula (XI-a):

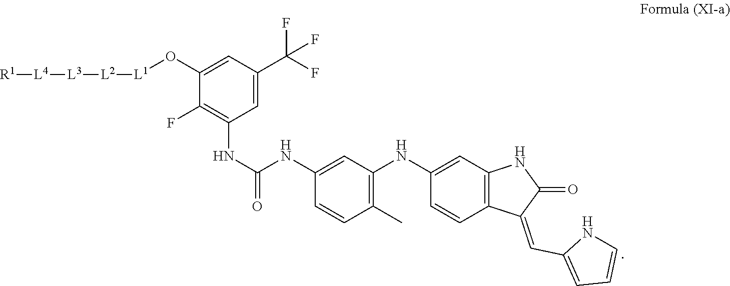

72. The compound, or a stereoisomer, tautomer, prodrug, or salt thereof, of any one of claims 1-71, wherein the TrkB ligand is a TrkB agonist, a TrkB partial agonist, or a TrkB antagonist.

73. The compound, or a stereoisomer, tautomer, prodrug, or salt thereof, of any one of claims 1-71, wherein the TrkB ligand is a TrkB selective ligand or a TrkB non-selective ligand.

74. The compound, or a stereoisomer, tautomer, prodrug, or salt thereof, of any one of claims 1-73, wherein L¹, L², L³, L⁴, L^{1′}, L^{2′}, L^{3′}, and L^{4′} are each independently absent, a bond, an optionally substituted alkyl linker, an optionally substituted polyethylene glycol (PEG) linker, an optionally substituted heteroalkyl linker, or an optionally substituted heteroaryl linker.

75. The compound, or a stereoisomer, tautomer, prodrug, or salt thereof, of claim 74, wherein L¹ and L^{1′} are each independently an optionally substituted heteroaryl linker.

76. The compound, or a stereoisomer, tautomer, prodrug, or salt thereof, of claim 74, wherein L¹ and L^{1′} are each independently an optionally substituted unsaturated heteroaryl, an optionally substituted heteroaryl or an optionally substituted saturated or partially unsaturated heterocycloalkyl linker.

77. The compound, or a stereoisomer, tautomer, prodrug, or salt thereof, of claim 75 or 76, wherein L¹ and L^{1′} each independently comprise the structure

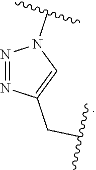 [SMILES: *Cc1cn(*)nn1]

78. The compound, or a stereoisomer, tautomer, prodrug, or salt thereof, of claim 74, wherein L¹ and L^{1′} are each independently an optionally substituted heteroalkyl linker.

79. The compound, or a stereoisomer, tautomer, prodrug, or salt thereof, of claim 78, wherein the optionally substituted heteroalkyl linker is an optionally substituted heteroalkyl or optionally substituted C₁-C₁₀ alkyl chain in which one or more carbon atoms are replaced with O, N, or S.

80. The compound, or a stereoisomer, tautomer, prodrug, or salt thereof, of claim 78 or 79, wherein L¹ and L^{1′} each independently comprise the structure

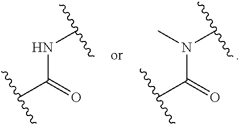

81. The compound, of salt thereof, of claim 78 or 79, wherein L¹ and L^{1′} each independently comprise the structure 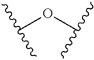 [SMILES: *O*]
or —N(CH₃)—.

82. The compound, or a stereoisomer, tautomer, prodrug, or salt thereof, of any one of claims 74-81, wherein L² and L^{2′} are each independently an optionally substituted PEG linker.

83. The compound, or a stereoisomer, tautomer, prodrug, or salt thereof, of claim 82, wherein the PEG linker is five PEG units in length.

84. The compound, or a stereoisomer, tautomer, prodrug, or salt thereof, of claim 82, wherein the PEG linker is four PEG units in length.

85. The compound, of salt thereof, of claim 82, wherein the PEG linker is three PEG units in length.

86. The compound, or a stereoisomer, tautomer, prodrug, or salt thereof, of any one of claims 74-81, wherein L² and L^{2′} are each independently an optionally substituted alkyl linker.

87. The compound, or a stereoisomer, tautomer, prodrug, or salt thereof, of claim 86, wherein L² and L^{2′} are each independently an optionally substituted C₁₋₂₀ alkyl linker.

88. The compound, or a stereoisomer, tautomer, prodrug, or salt thereof, of claim 86 or 87, wherein L² and L^{2′} are each independently an optionally substituted C₈ alkyl linker.

89. The compound, or a stereoisomer, tautomer, prodrug, or salt thereof, of any one of claims 76-88, wherein L³ and L^{3′} are each independently is an optionally substituted heteroaryl linker.

90. The compound, or a stereoisomer, tautomer, prodrug, or salt thereof, of claim 89, wherein L³ and L^{3′} are each independently an optionally substituted partially unsaturated heteroaryl linker, an optionally substituted heteroaryl or an optionally substituted saturated or partially unsaturated heterocycloalkyl linker.

91. The compound, or a stereoisomer, tautomer, prodrug, or salt thereof, of claim 90, wherein L³ and L^{3′} each independently comprise the structure

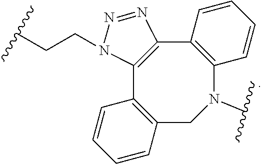 [SMILES: *CCn1nnc2c1-c1ccccc1CN(*)c1ccccc1-2]

92. The compound, or a stereoisomer, tautomer, prodrug, or salt thereof, of any one of claims 74-91, wherein L⁴ and L^{4′} are each independently an optionally substituted heteroalkyl linker.

93. The compound, or a stereoisomer, tautomer, prodrug, or salt thereof, of claim 92, wherein the heteroalkyl linker is substituted with one or more ═O substituents.

94. The compound, or a stereoisomer, tautomer, prodrug, or salt thereof, of claim 92 or 93, wherein the heteroalkyl linker comprises two substituents joined together to form an optionally substituted carbocyclyl ring.

95. The compound, or a stereoisomer, tautomer, prodrug, or salt thereof, of any one of claims 92-94, wherein L⁴ and L^{4′} each independently comprise the structure 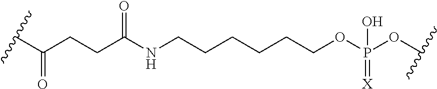
or a salt thereof,
wherein X is O or S.

96. The compound, or a stereoisomer, tautomer, prodrug, or salt thereof, of any one of claims 92-94, wherein L⁴ and L^{4′} each independently comprise the structure: 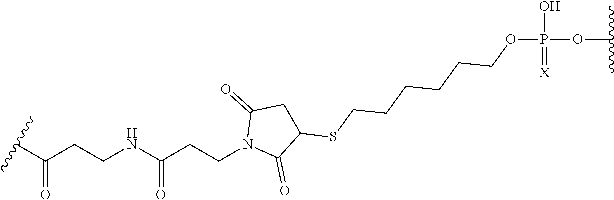
or a salt thereof, wherein X is O or S.

97. The compound, or a stereoisomer, tautomer, prodrug, or salt thereof, of any one of claims 1-96, wherein L¹-L²-L³-L⁴; or L^{1′}-L^{2′}-L^{3′}-L^{4′} each independently comprise the structure: 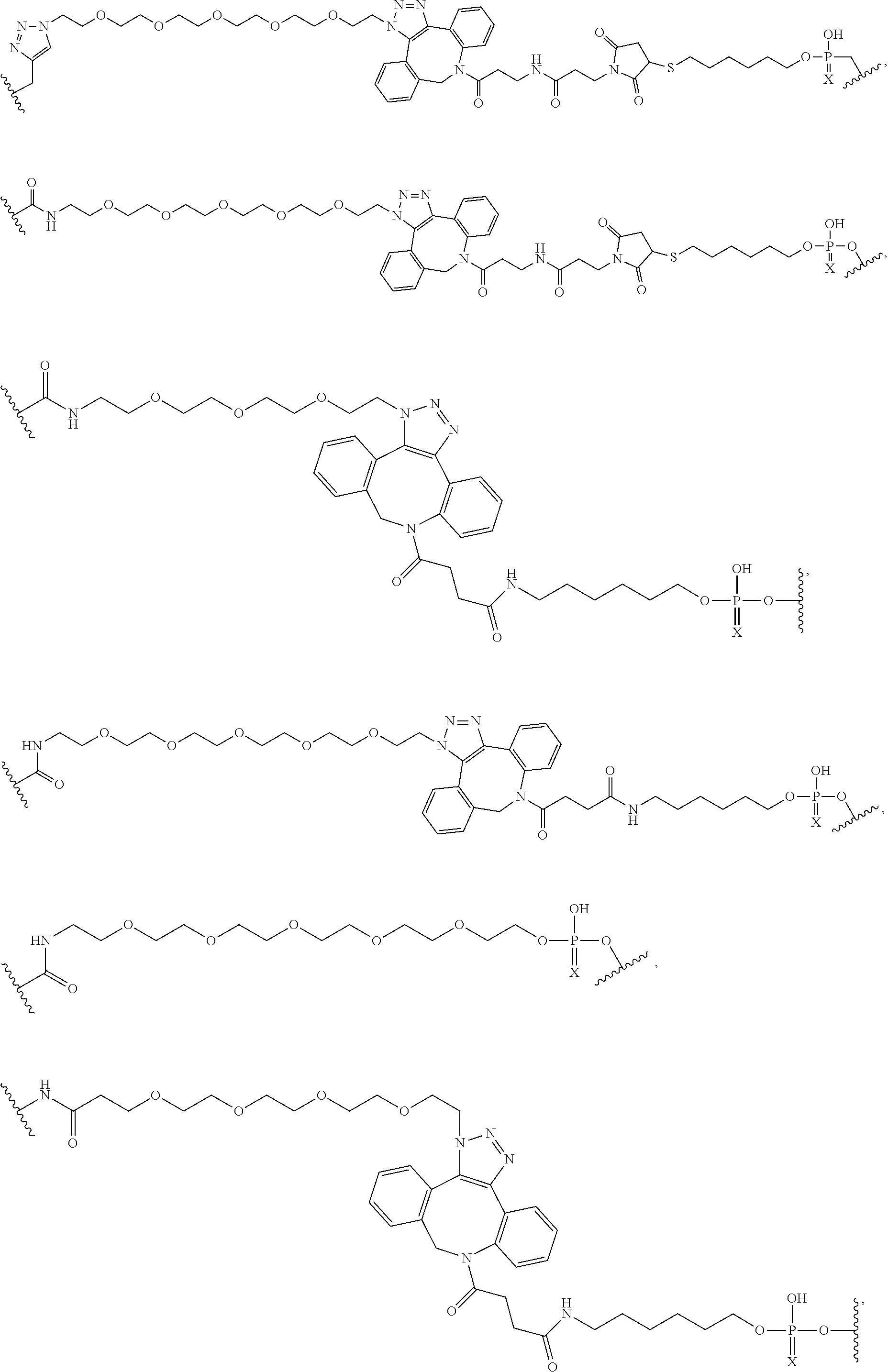 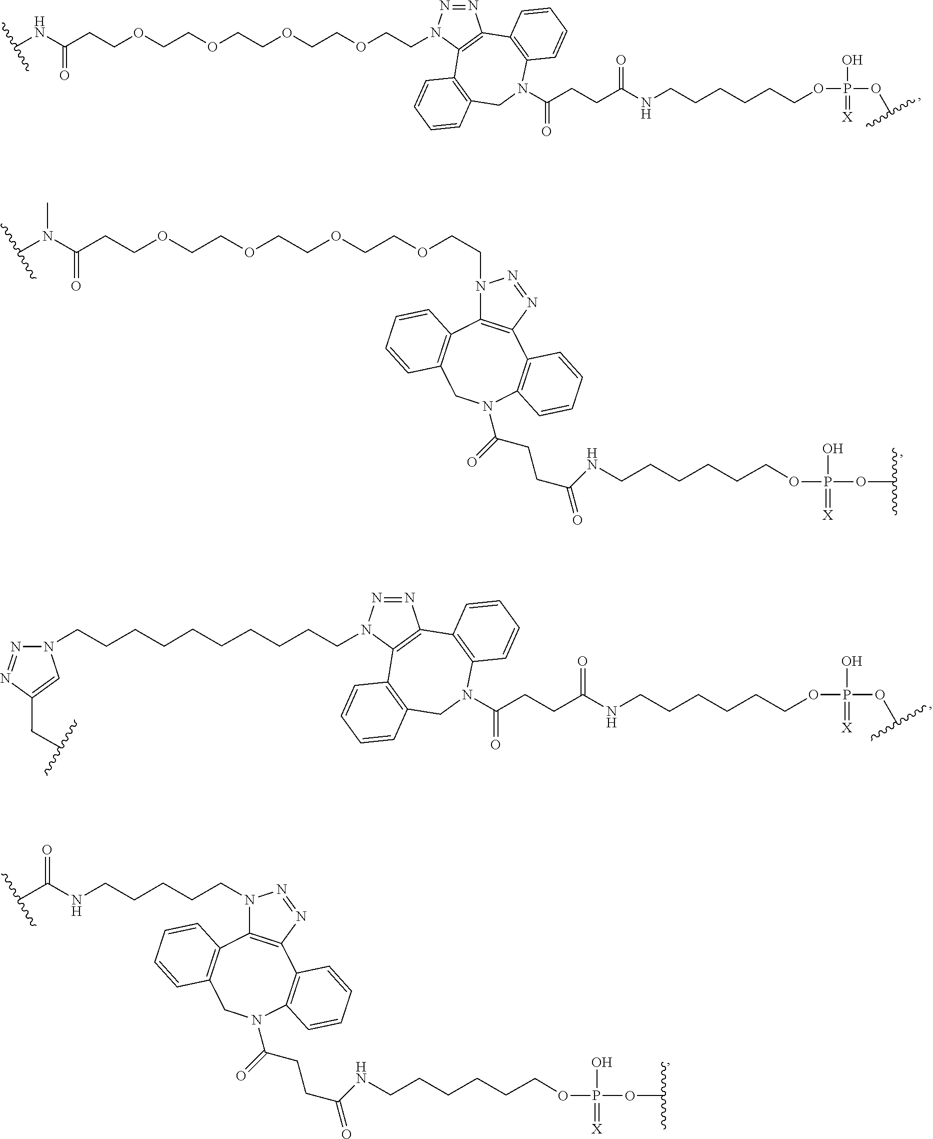 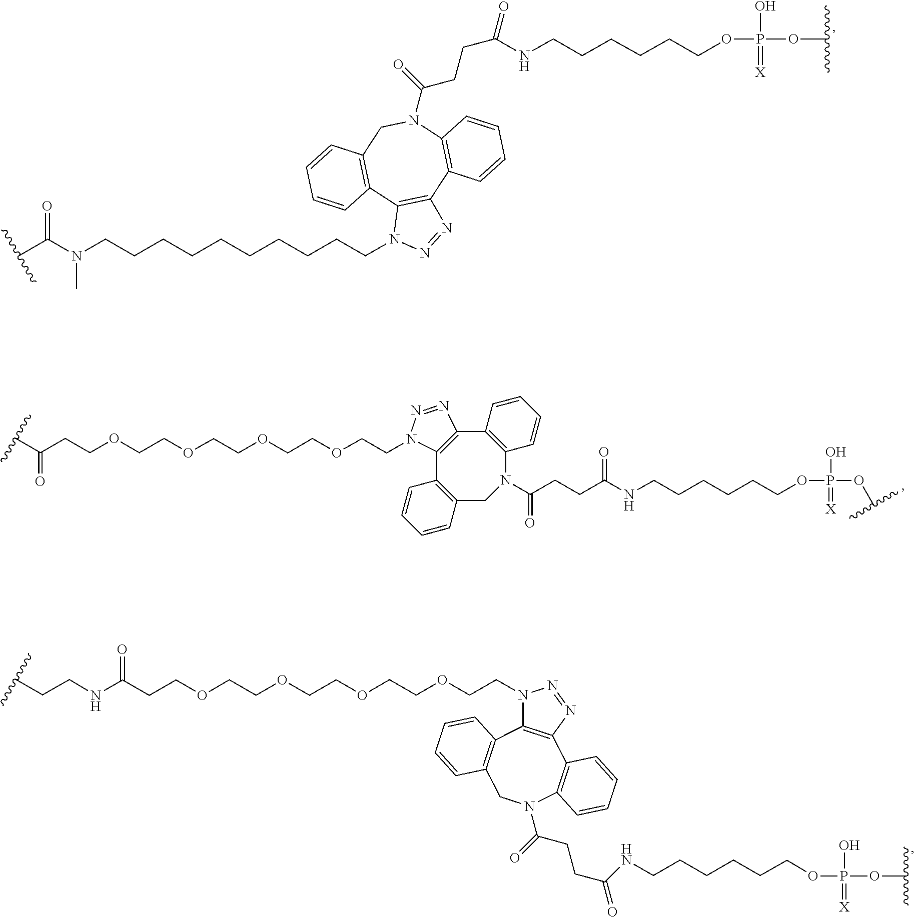 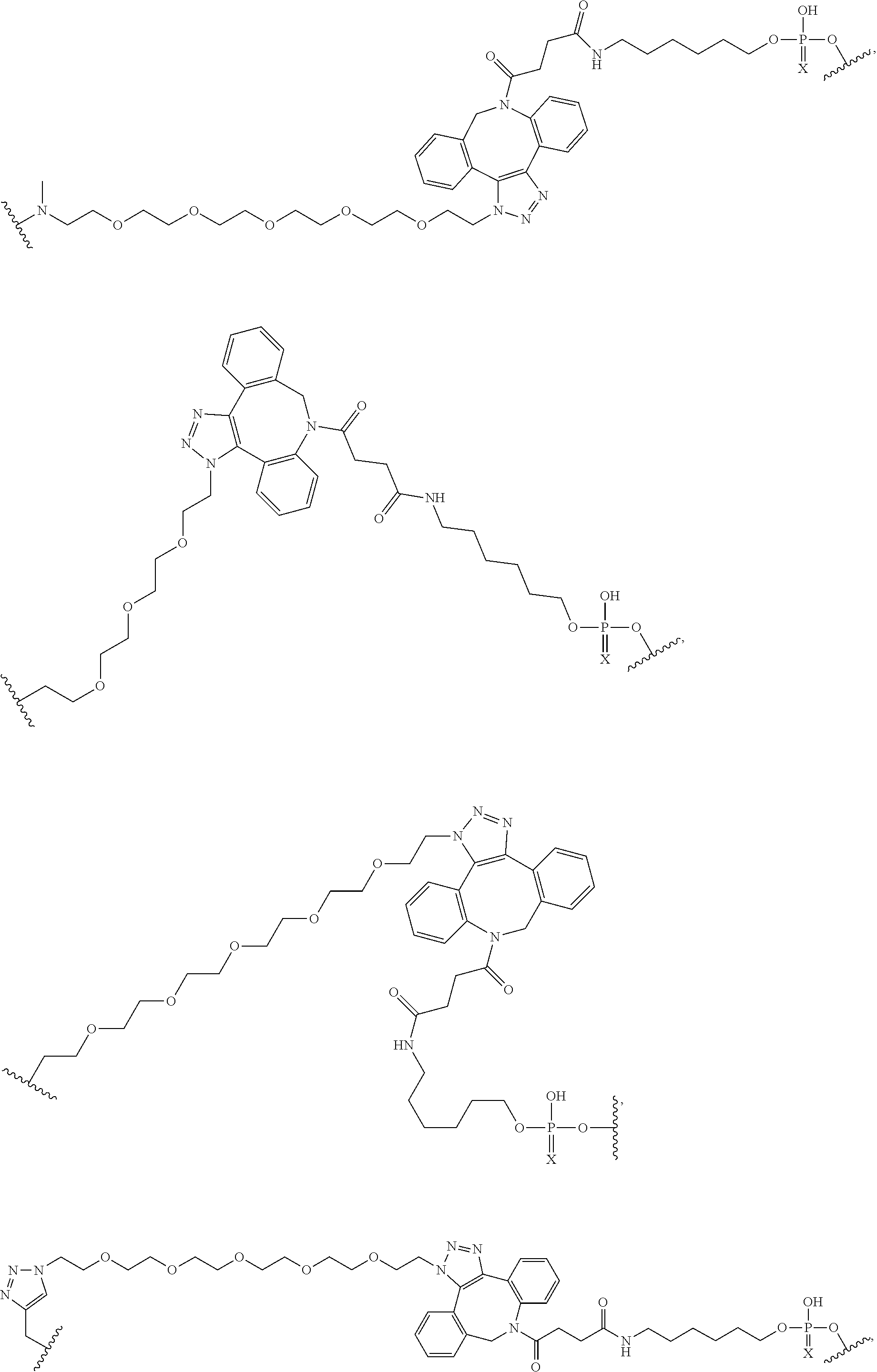 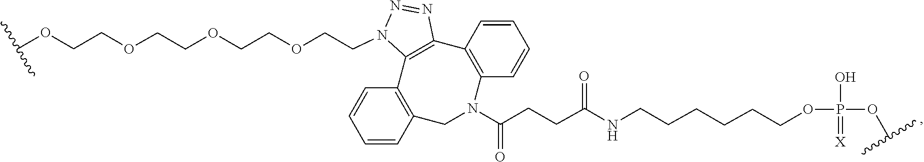
or a salt thereof, wherein X is O or S.

98. A compound, or a stereoisomer, tautomer, prodrug, or salt thereof, wherein the compound comprises the structure: 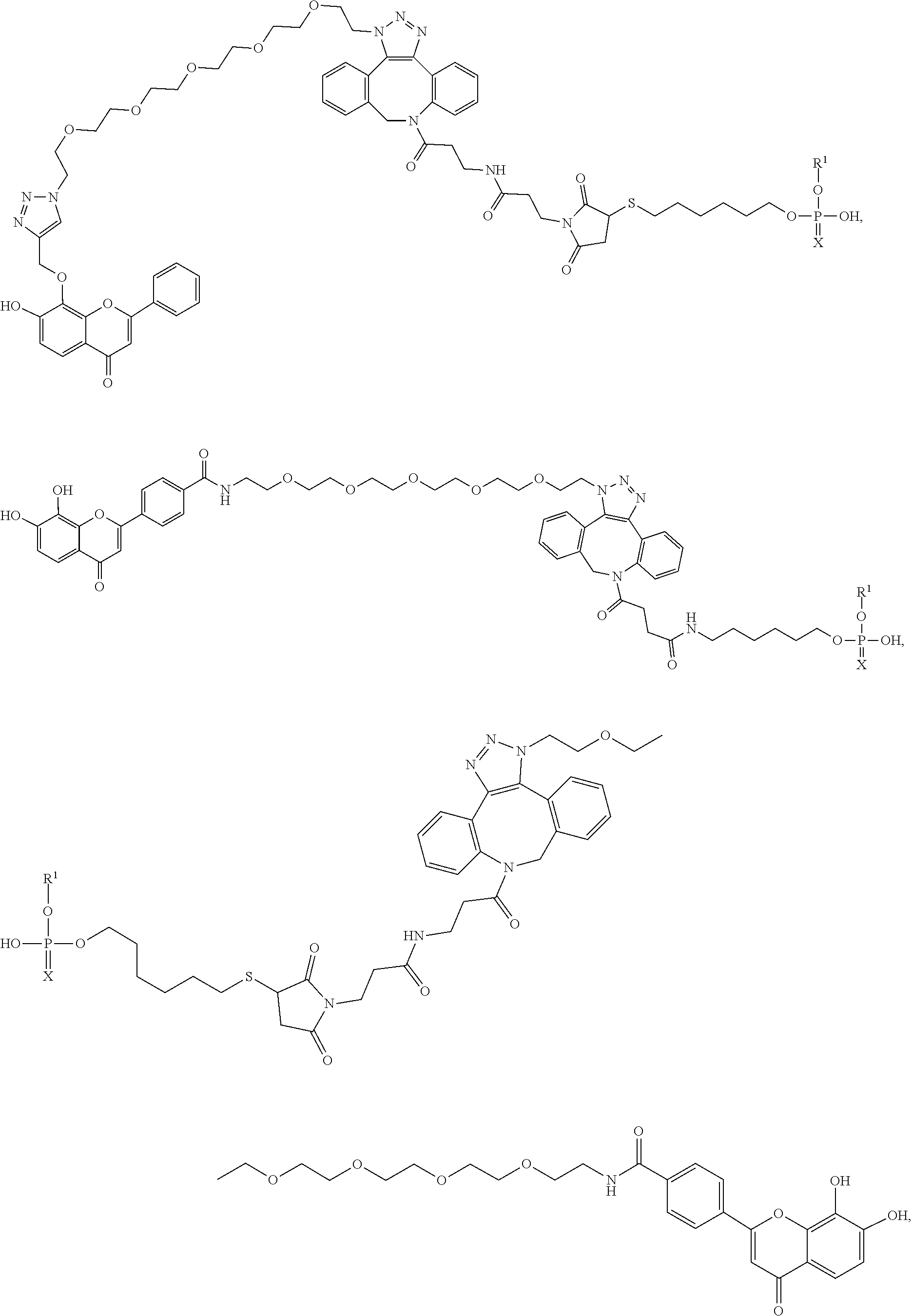 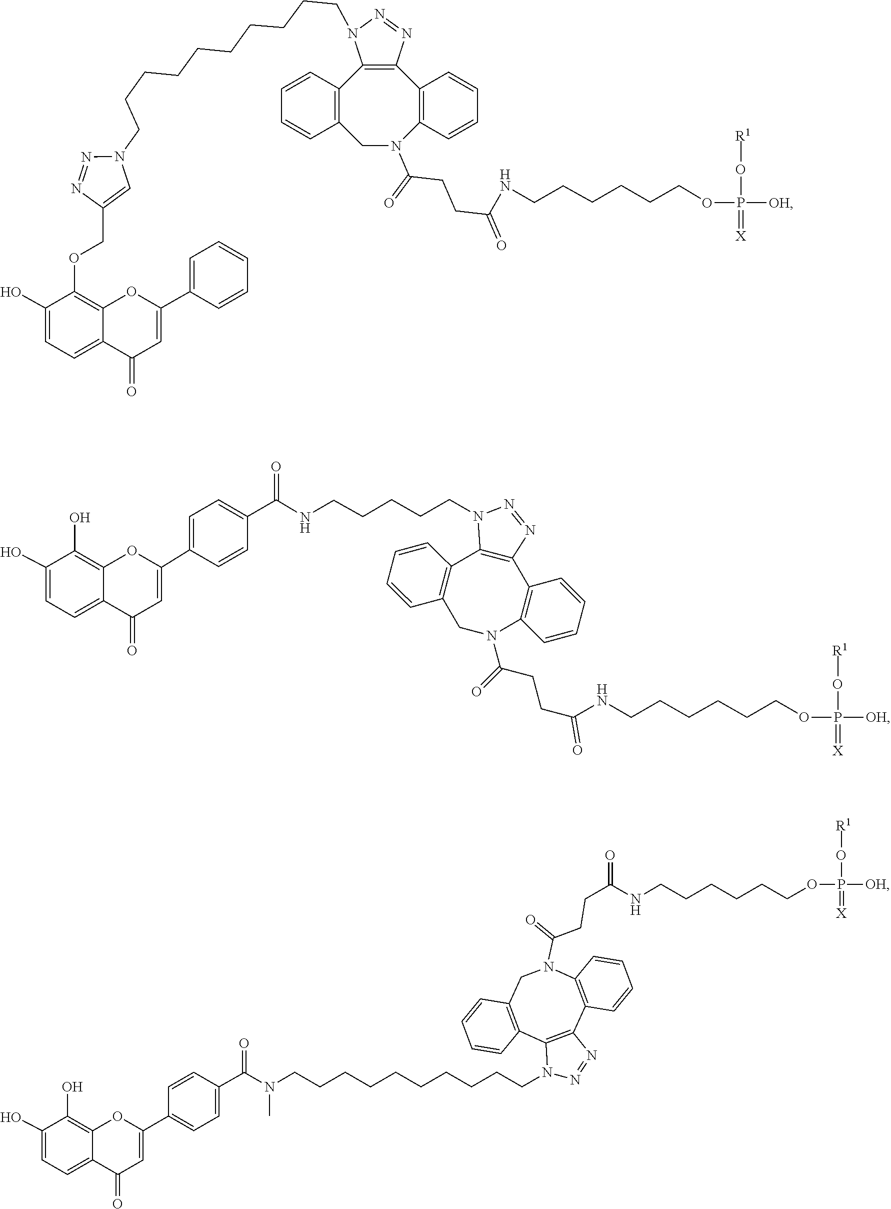 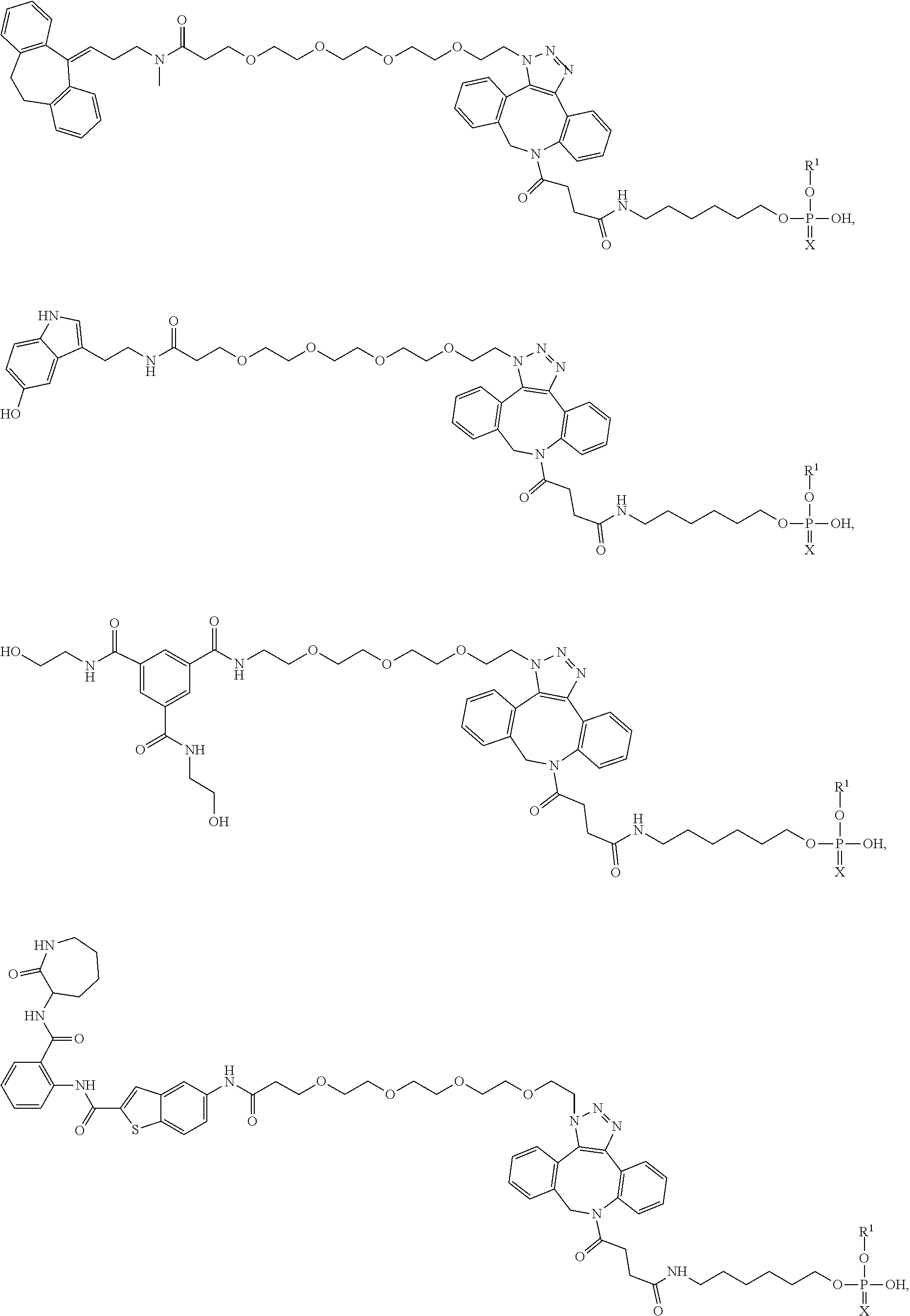 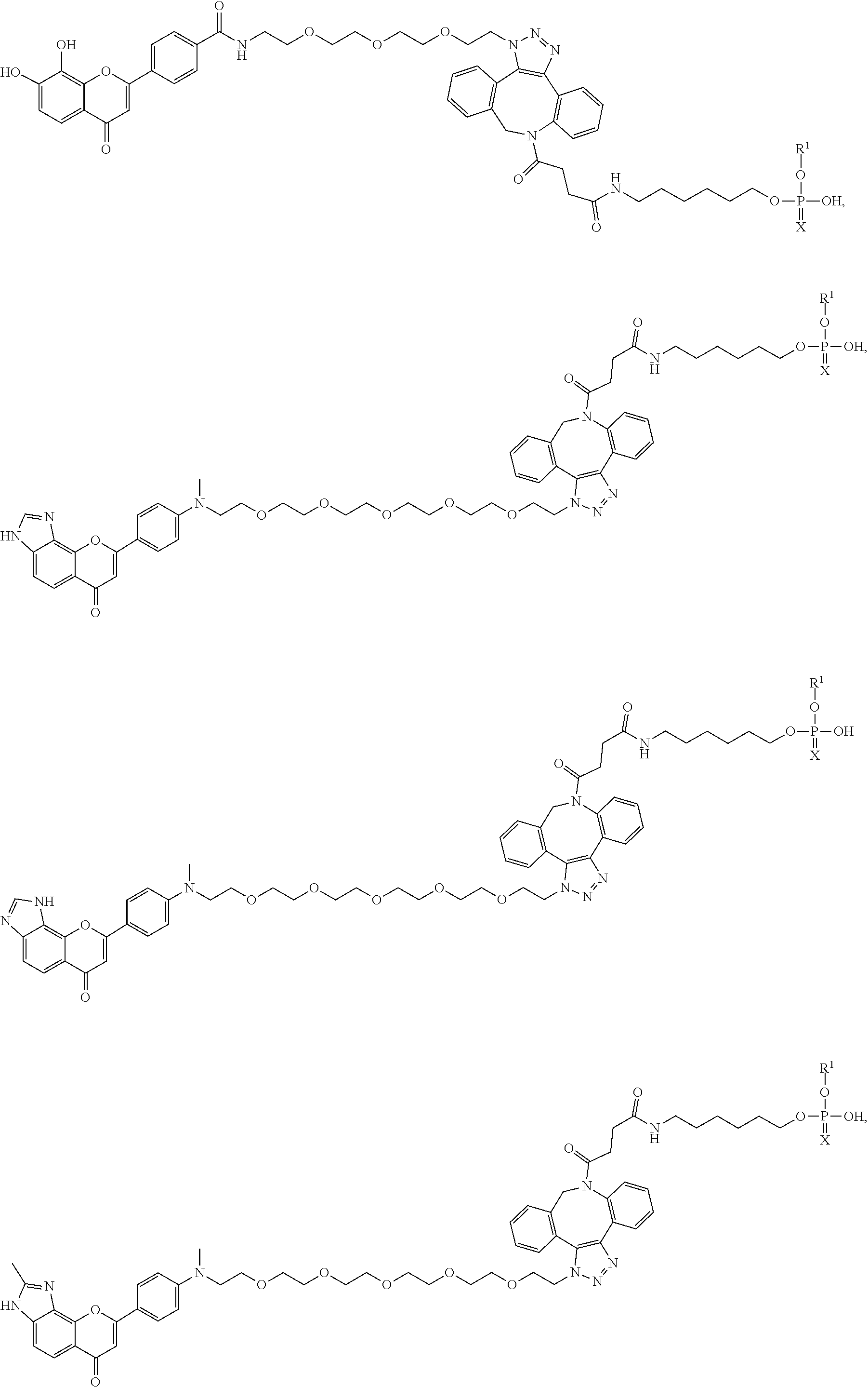 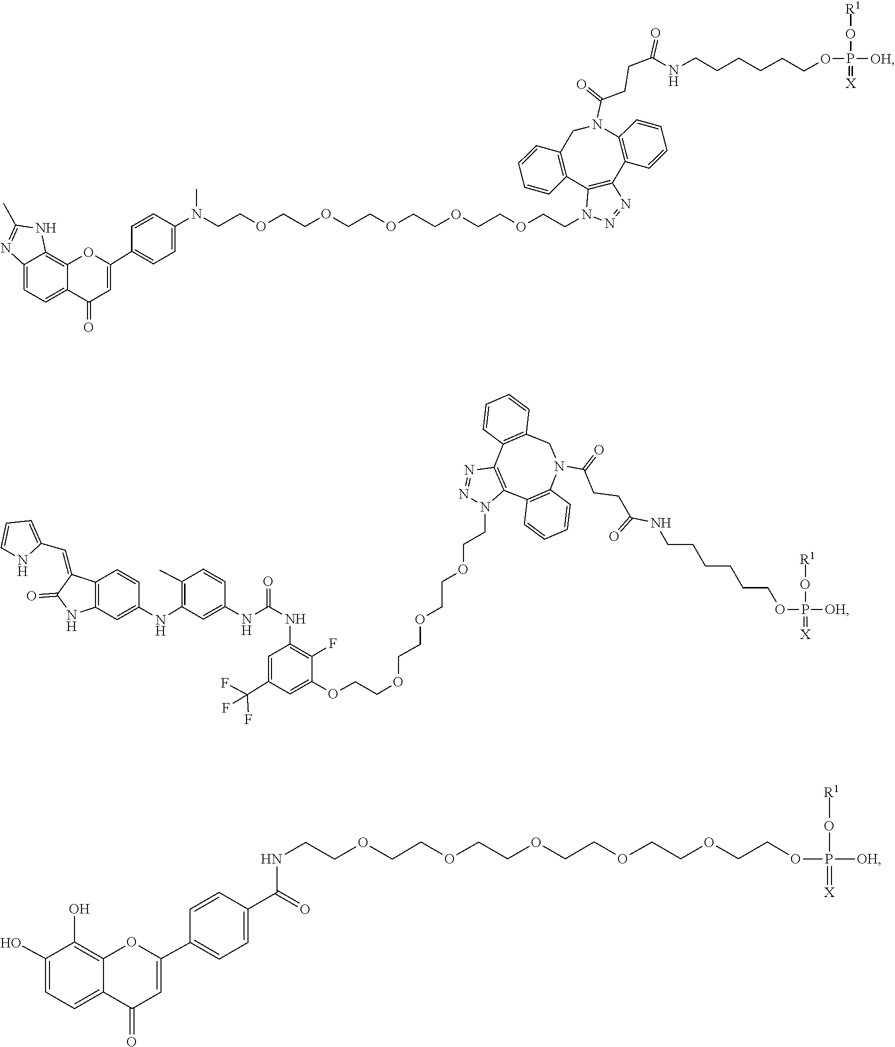 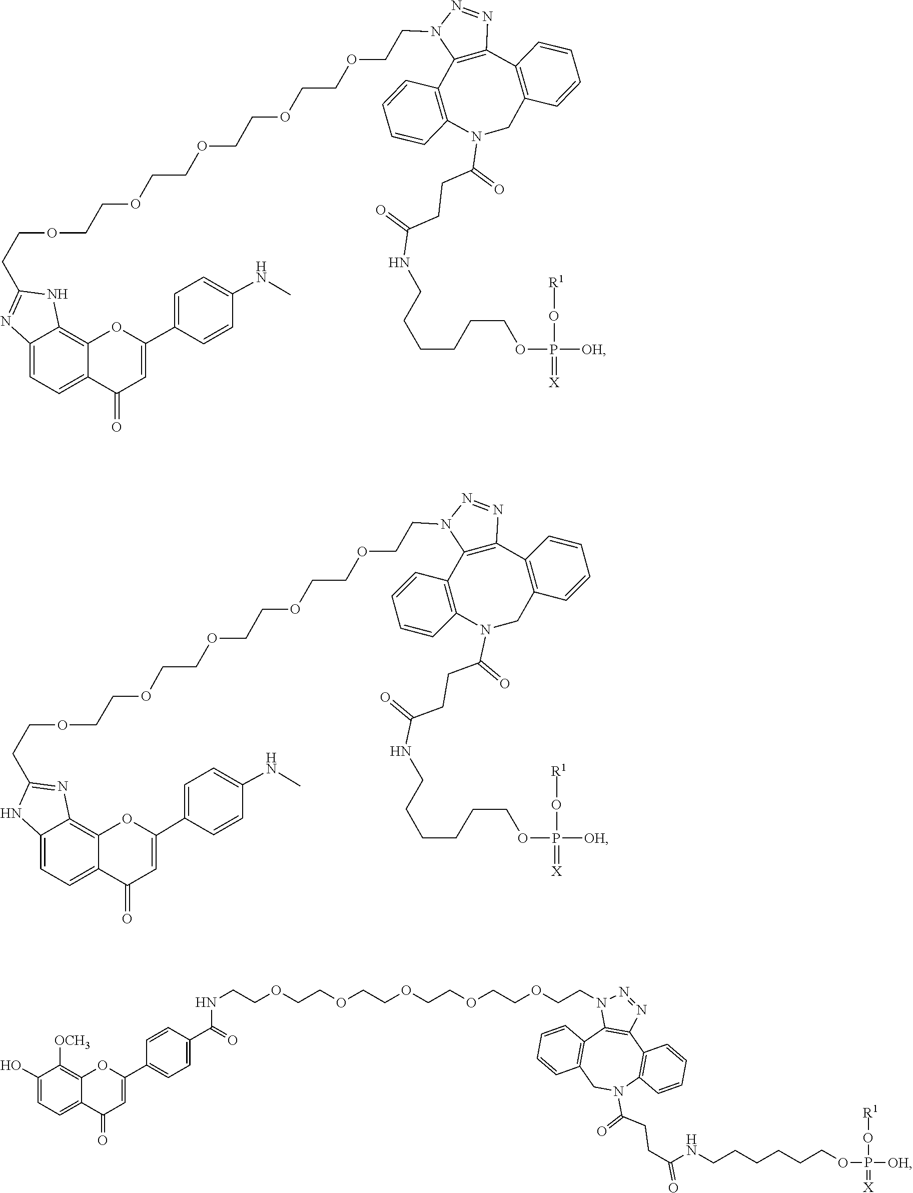 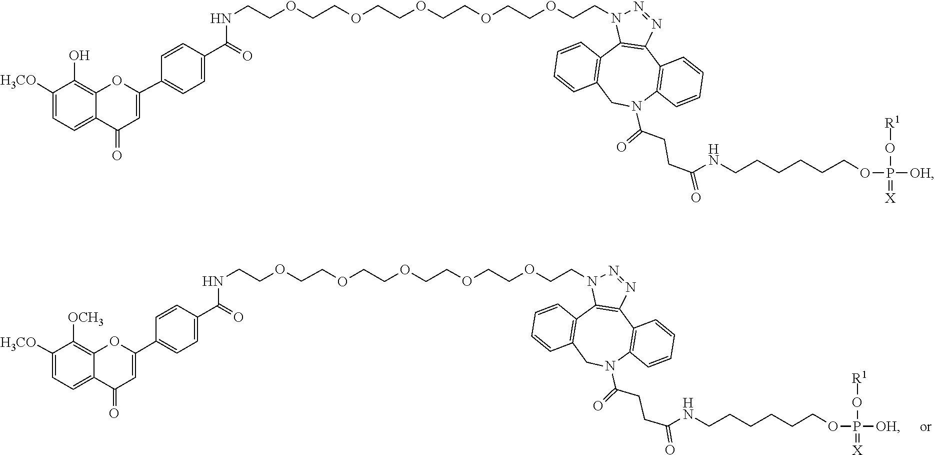 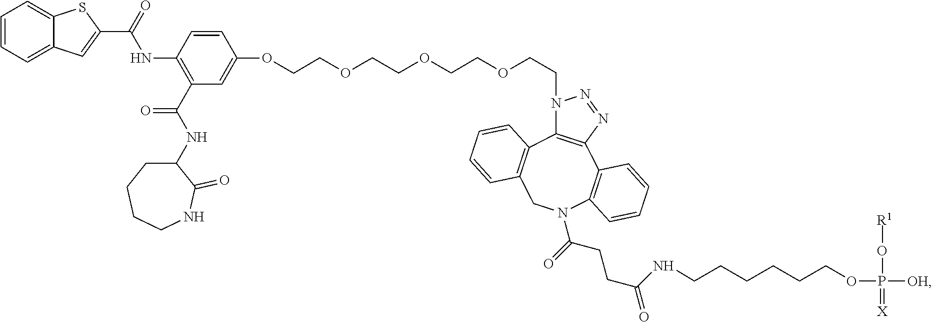
wherein:
R¹ is an oligonucleotide, a protecting group, a small molecule, a protein, an antibody, or a peptide; and
X is O or S.

99. A compound, or a stereoisomer, tautomer, prodrug, or salt thereof, wherein the compound comprises the structure: 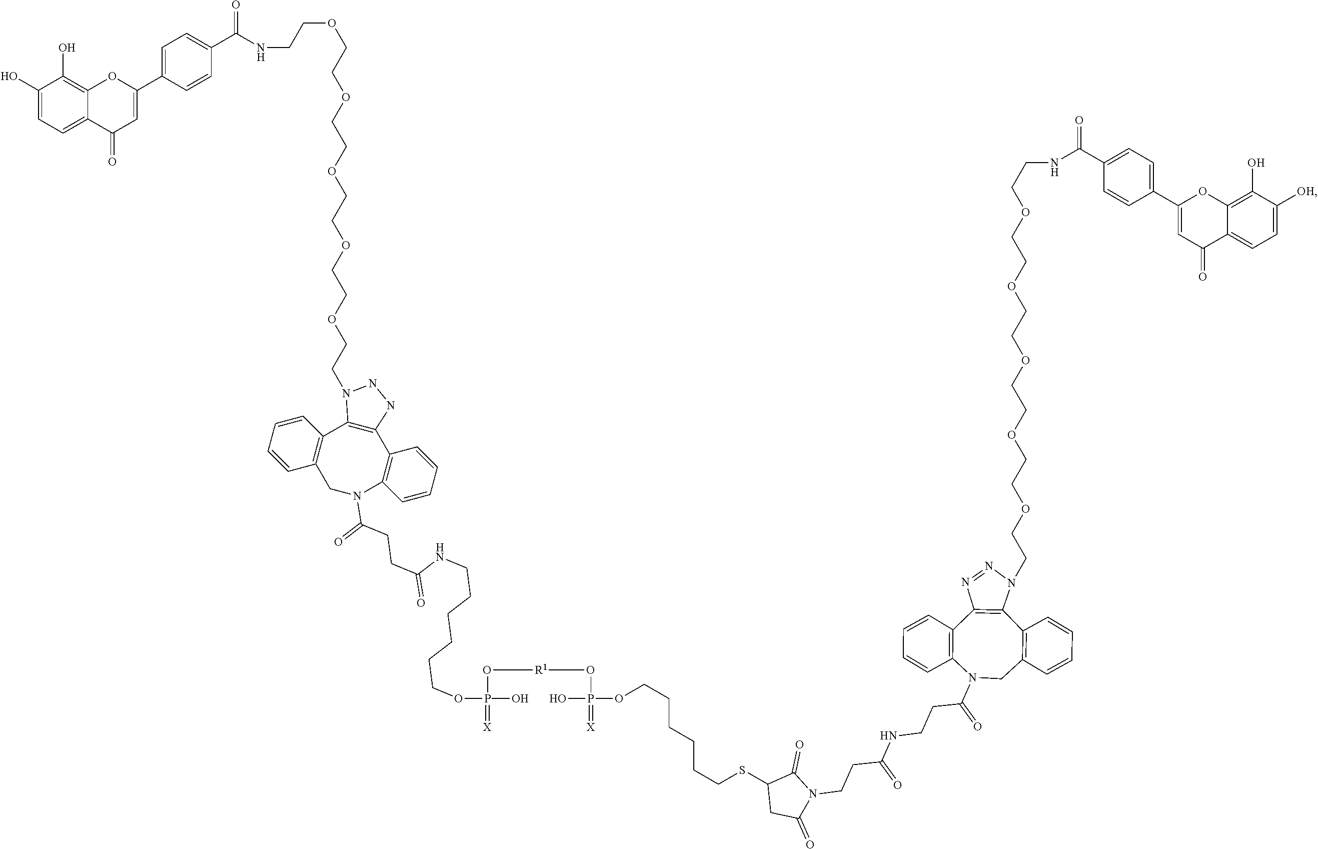 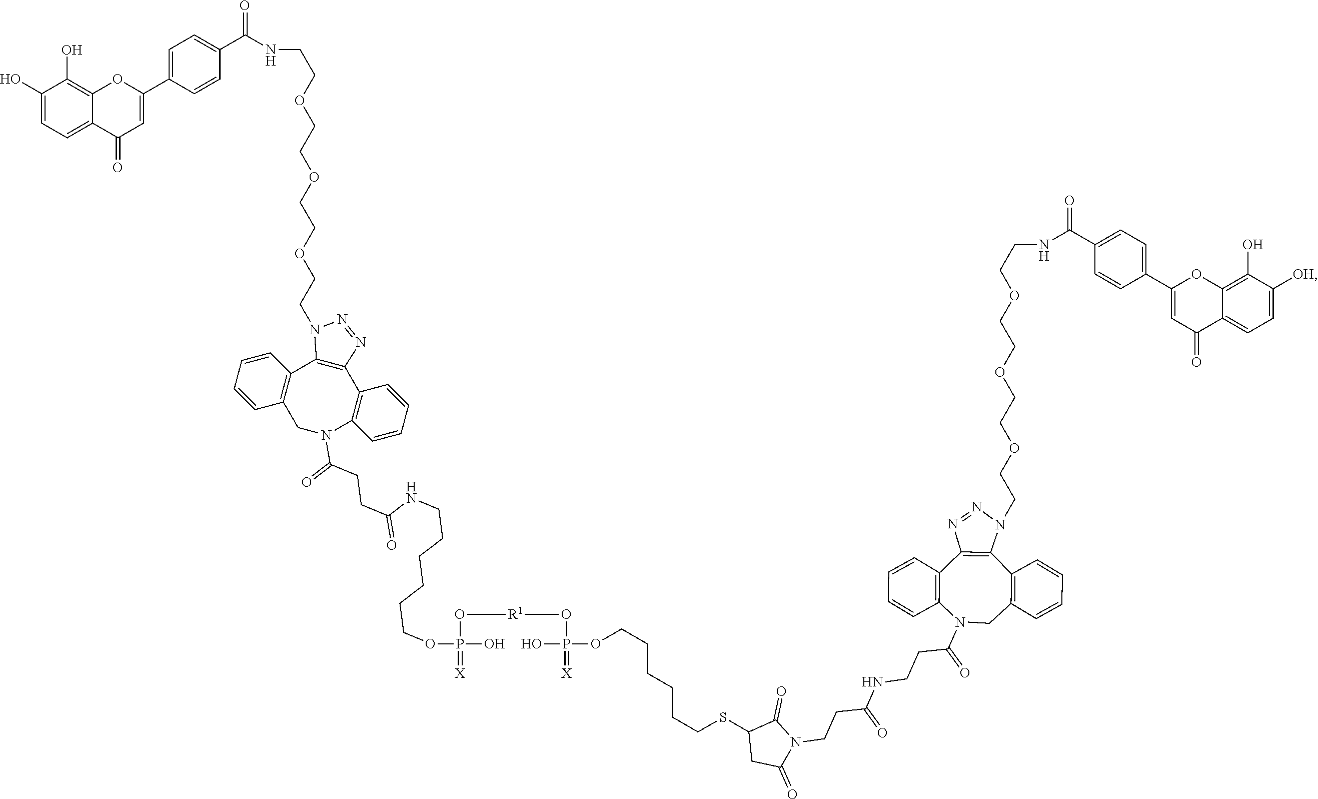 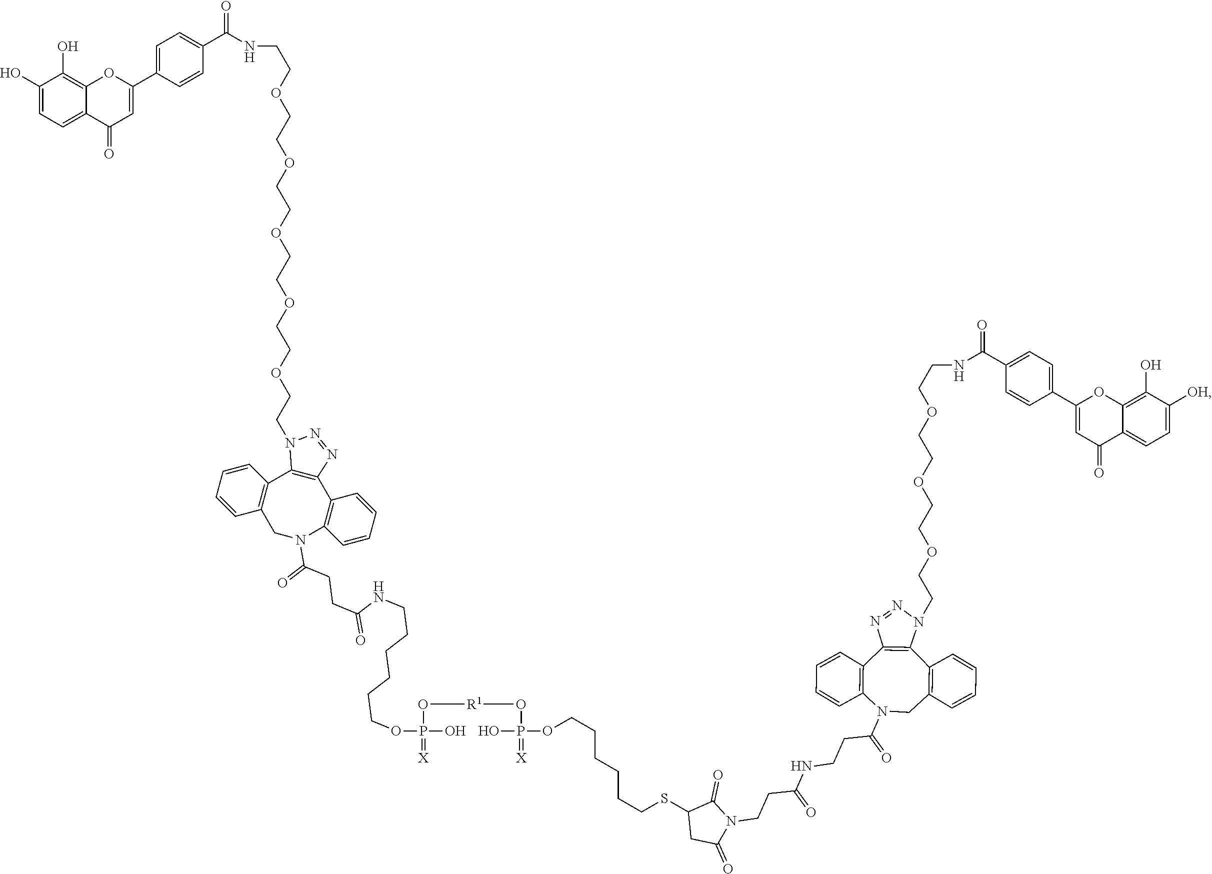 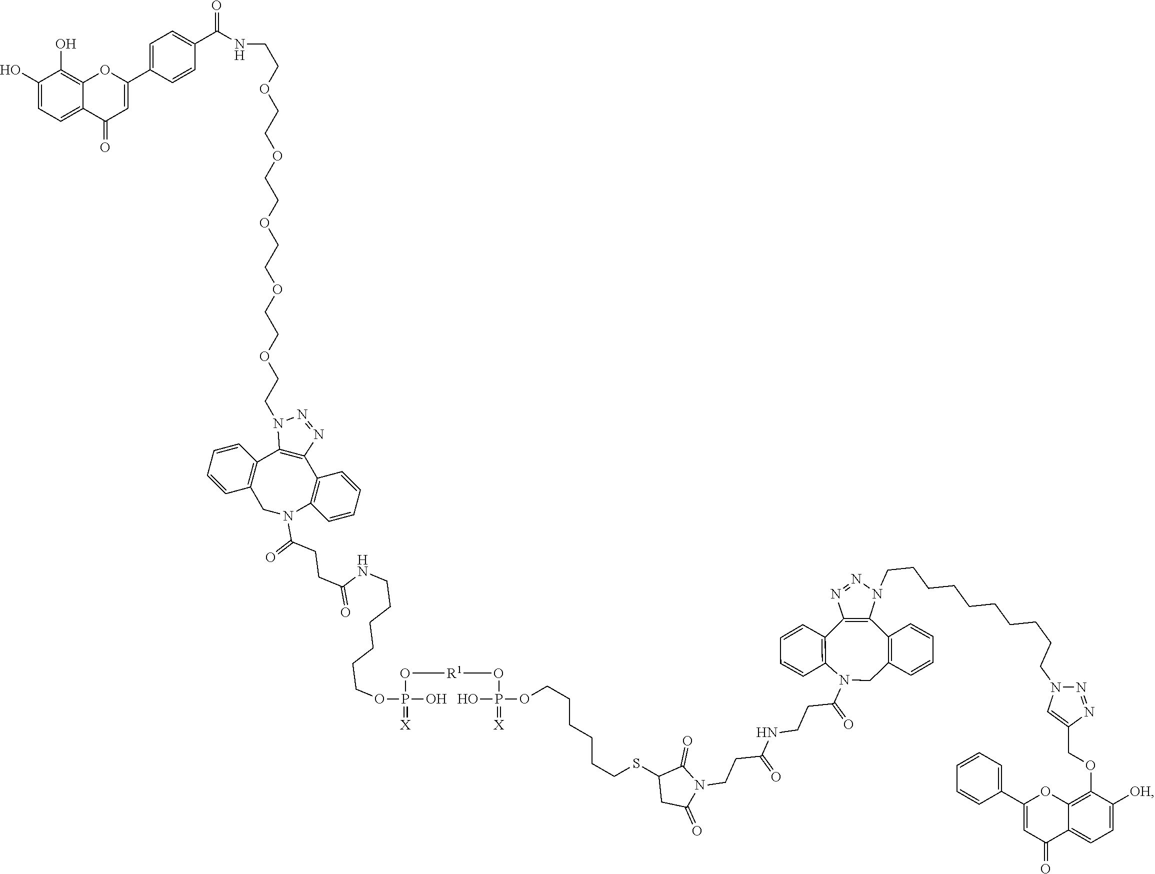 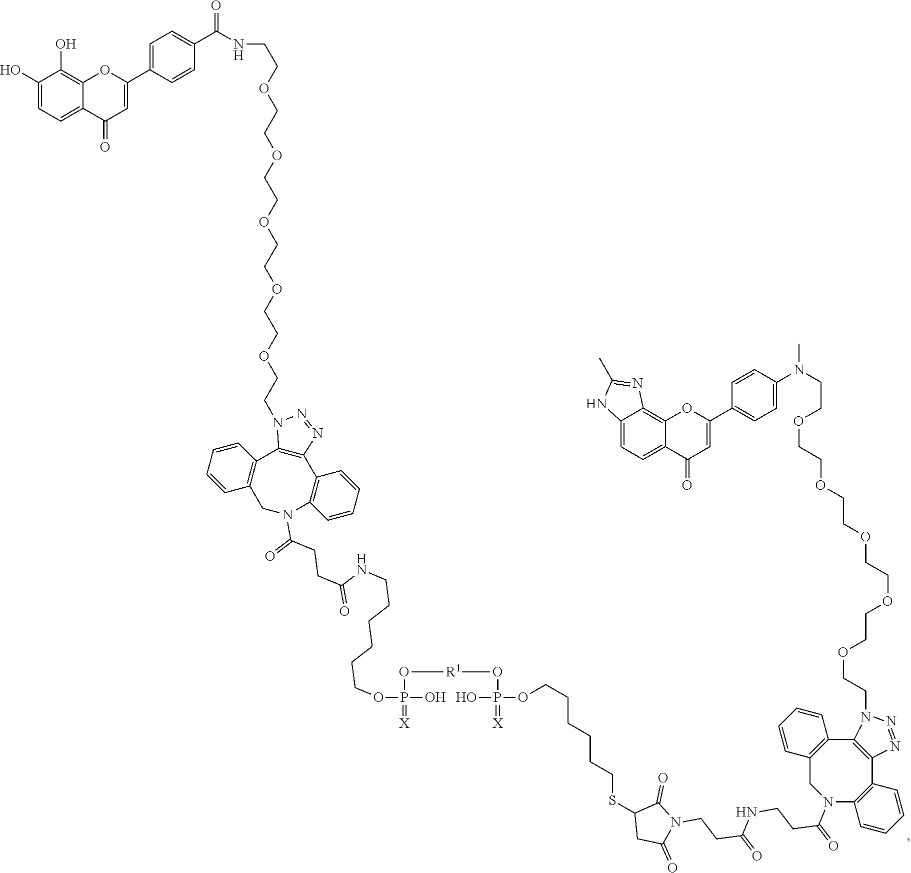 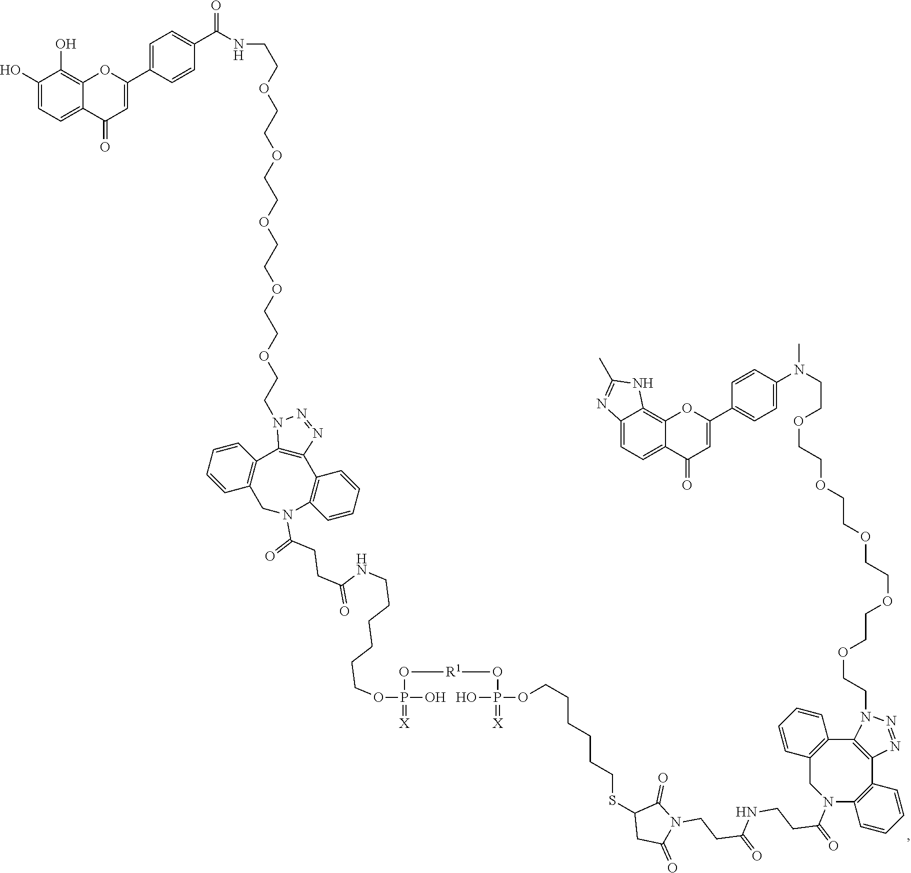 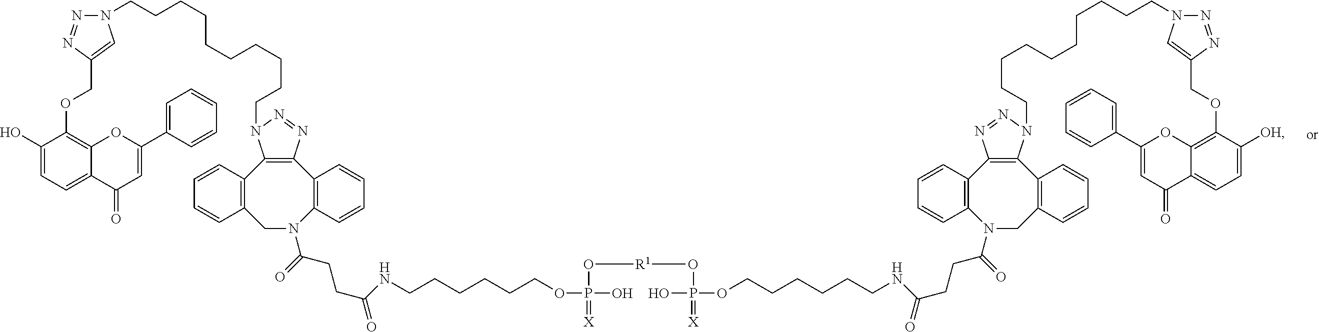 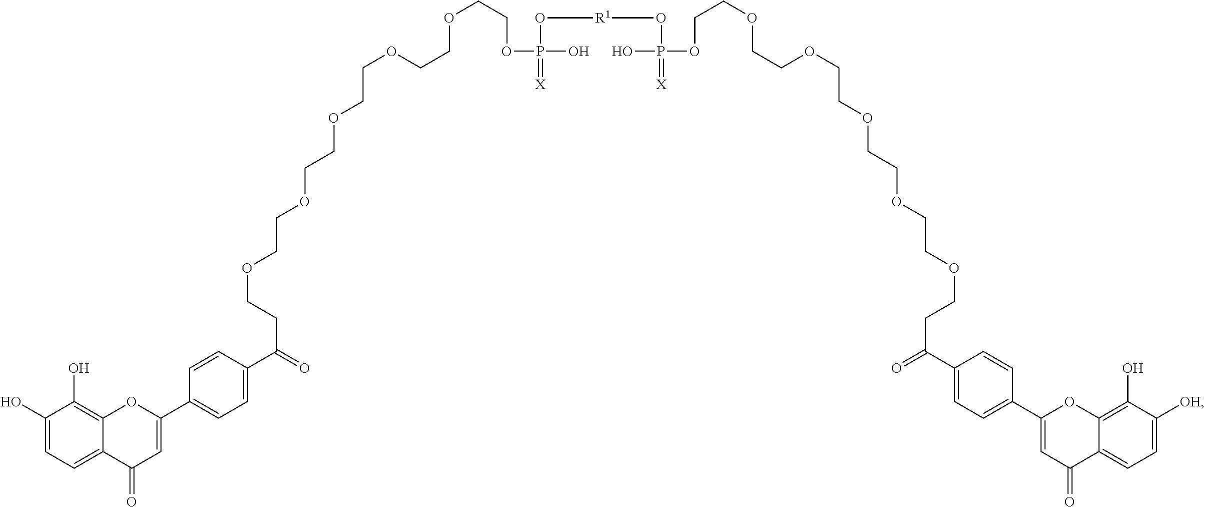
wherein:
R¹ is an oligonucleotide, a protecting group, a small molecule, a protein, an antibody, or a peptide; and
X is O or S.

100. A compound, or a stereoisomer, tautomer, prodrug, or salt thereof, wherein the compound comprises the structure: 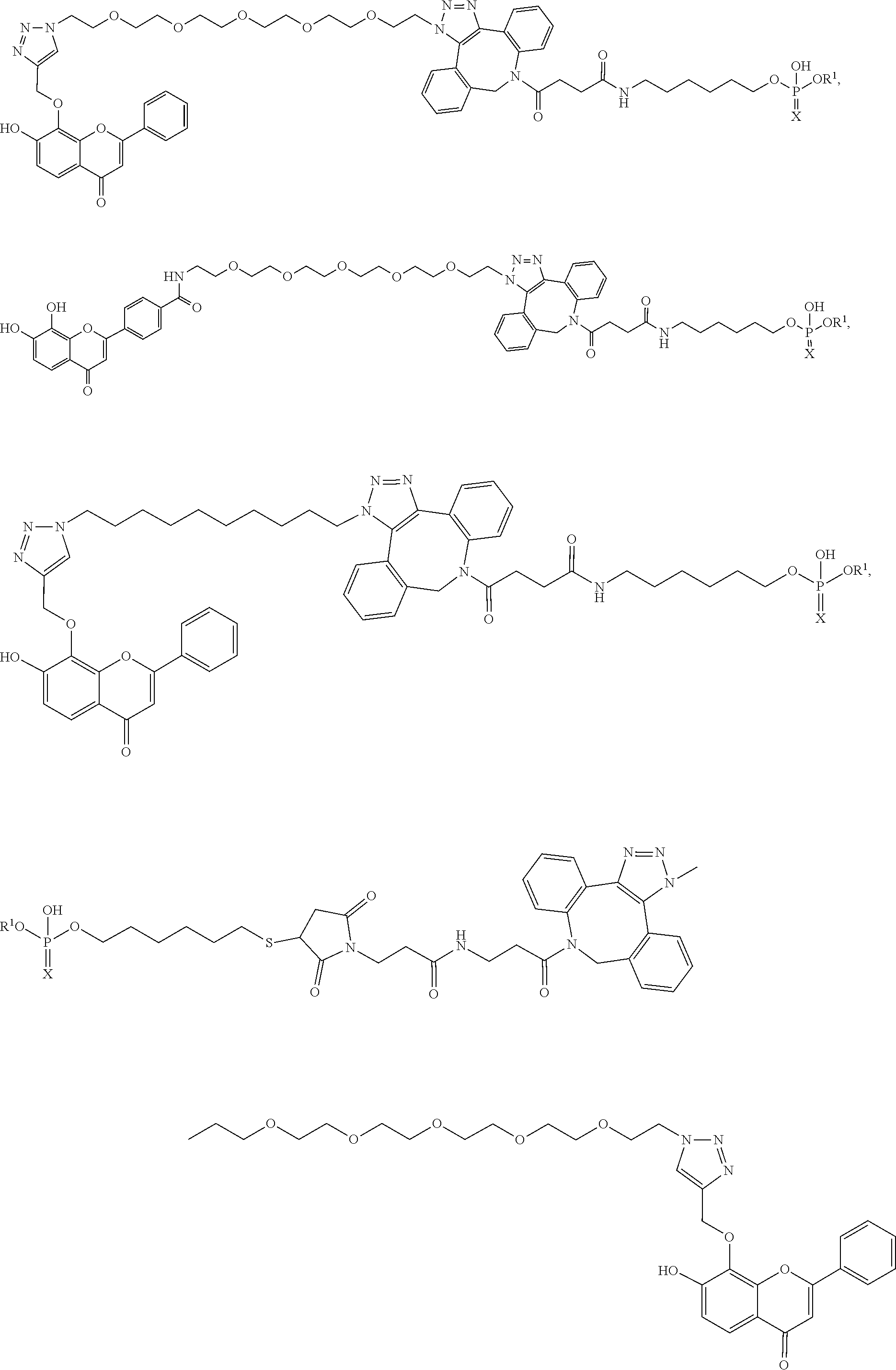
wherein:
R¹ is an oligonucleotide, a protecting group, a small molecule, a protein, an antibody, or a peptide; and
X is O or S.

101. A compound, or a stereoisomer, tautomer, prodrug, or salt thereof, wherein the compound comprises the structure: 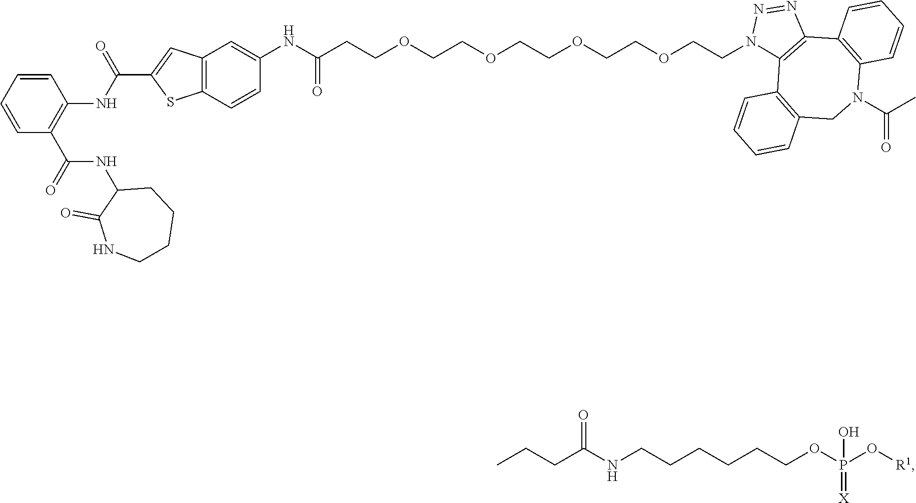
wherein:
R¹ is an oligonucleotide, a protecting group, a small molecule, a protein, an antibody, or a peptide; and
X is O or S.

102. The compound, or a stereoisomer, tautomer, prodrug, or salt thereof, of any one of claims 97-101, wherein X is O.

103. The compound, or a stereoisomer, tautomer, prodrug, or salt thereof, of any one of claims 97-101, wherein X is S.

104. The compound, or a stereoisomer, tautomer, prodrug, or salt thereof, of any one of claims 1-103, wherein R¹ comprises an oligonucleotide.

105. The compound, or a stereoisomer, tautomer, prodrug, or salt thereof, of claim 104, wherein the oligonucleotide is attached at its 5′ end.

106. The compound, or a stereoisomer, tautomer, prodrug, or salt thereof, of claim 104, wherein the oligonucleotide is attached at its 3′ end.

107. The compound, or a stereoisomer, tautomer, prodrug, or salt thereof, of claim 104, wherein the oligonucleotide is attached at an internal position on the oligonucleotide.

108. The compound, or a stereoisomer, tautomer, prodrug, or salt thereof, of claim 104, wherein the internal position is an internucleoside linkage.

109. The compound, or a stereoisomer, tautomer, prodrug, or salt thereof, of any one of claims 1-108, wherein R¹ comprises an oligonucleotide conjugated to one or more additional TrkB ligands.

110. The compound, or a stereoisomer, tautomer, prodrug, or salt thereof, of claim 109, wherein the oligonucleotide is conjugated to two, three, four, five, or more than five additional TrkB ligands.

111. The compound, or a stereoisomer, tautomer, prodrug, or salt thereof, of claim 109 or 110, wherein the additional TrkB ligands are conjugated to the oligonucleotide at the 5′ end of the oligonucleotide, the 3′ end of the oligonucleotide, the 5′- and 3′ ends of the oligonucleotide, one or more internal positions on the oligonucleotide, or any combination thereof.

112. The compound, or a stereoisomer, tautomer, prodrug, or salt thereof, of any one of claims 1-111, wherein the oligonucleotide is a modified oligonucleotide.

113. A compound, or a stereoisomer, tautomer, prodrug, or salt thereof, wherein the compound comprises the structure: 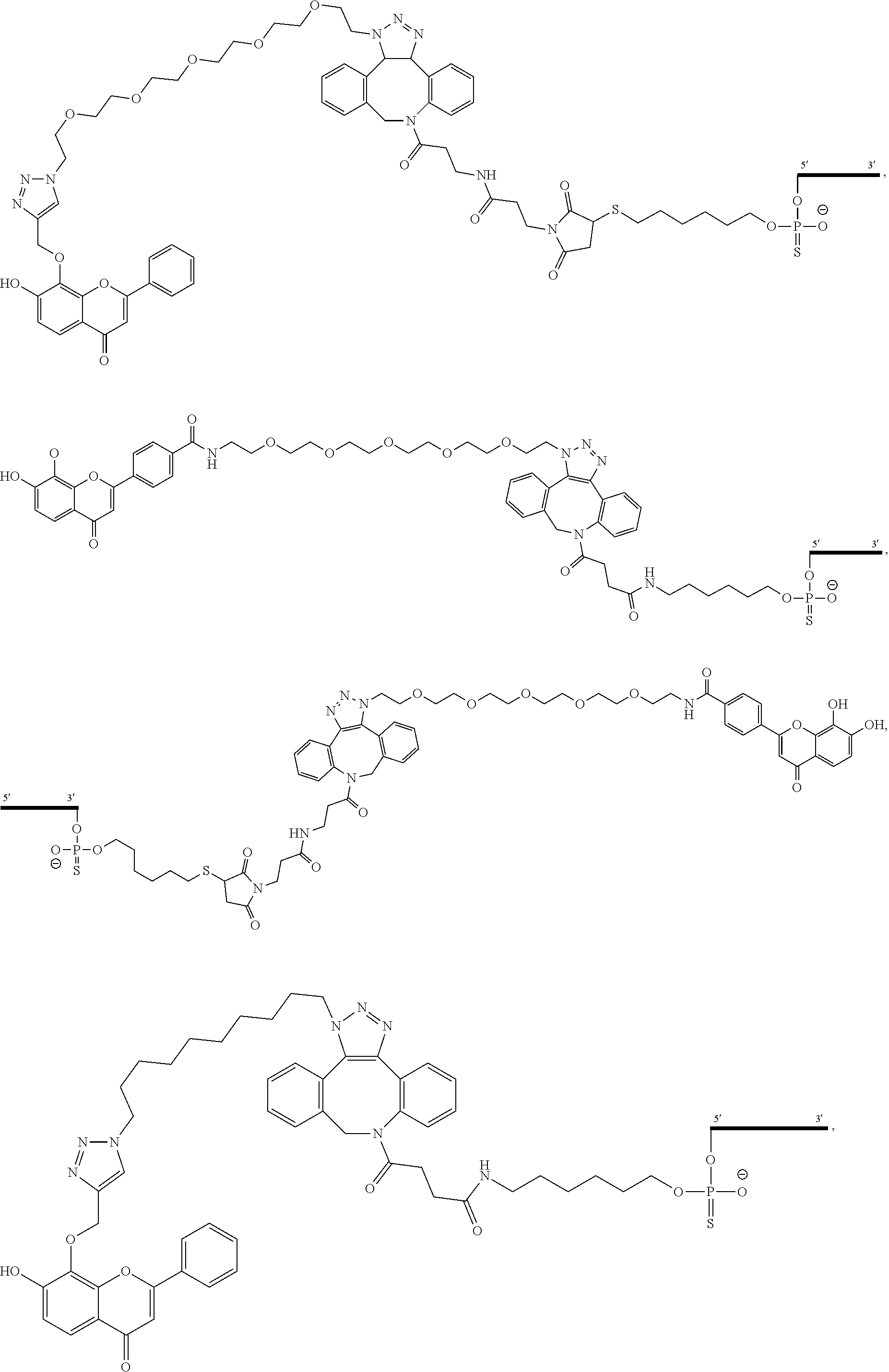 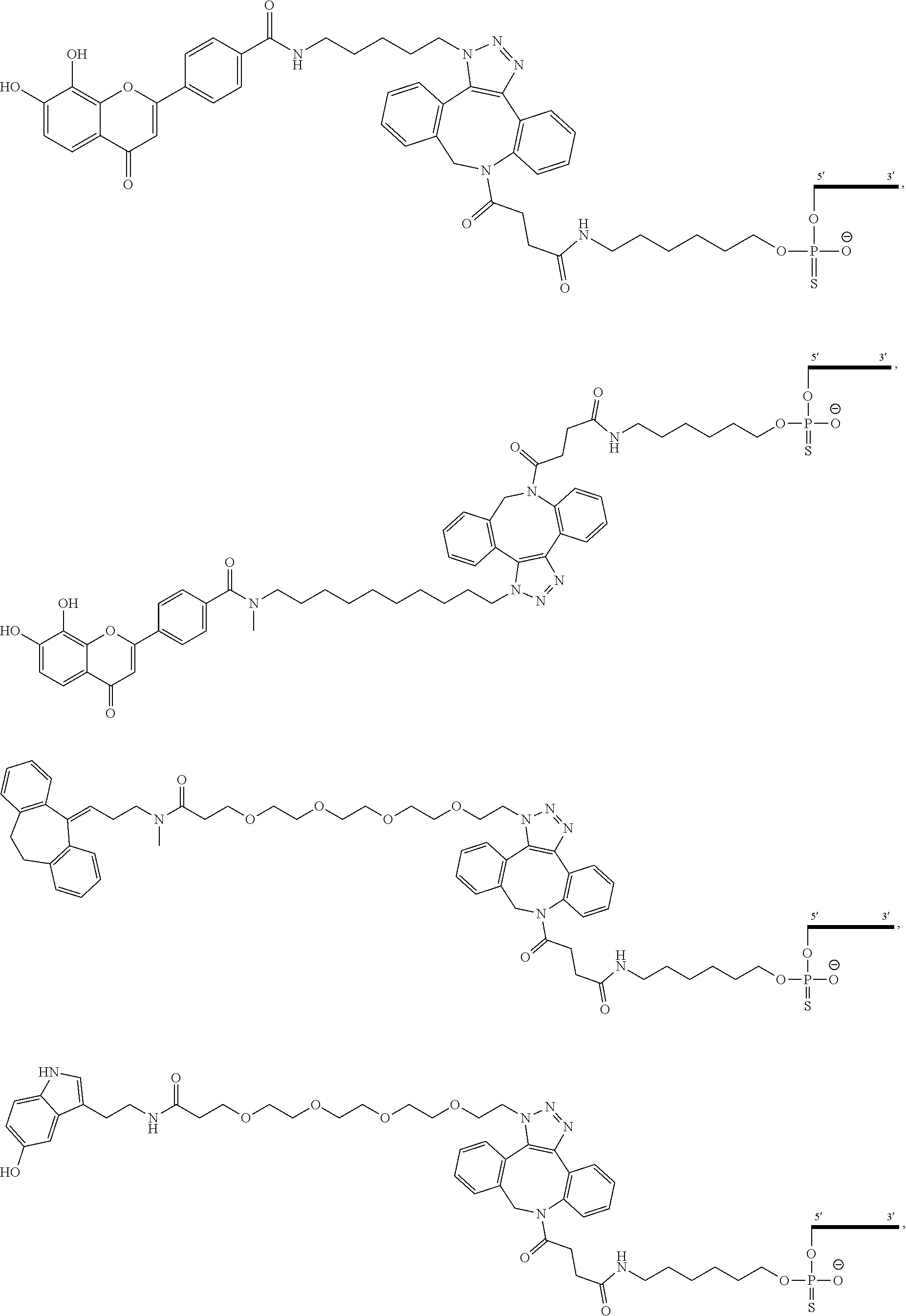 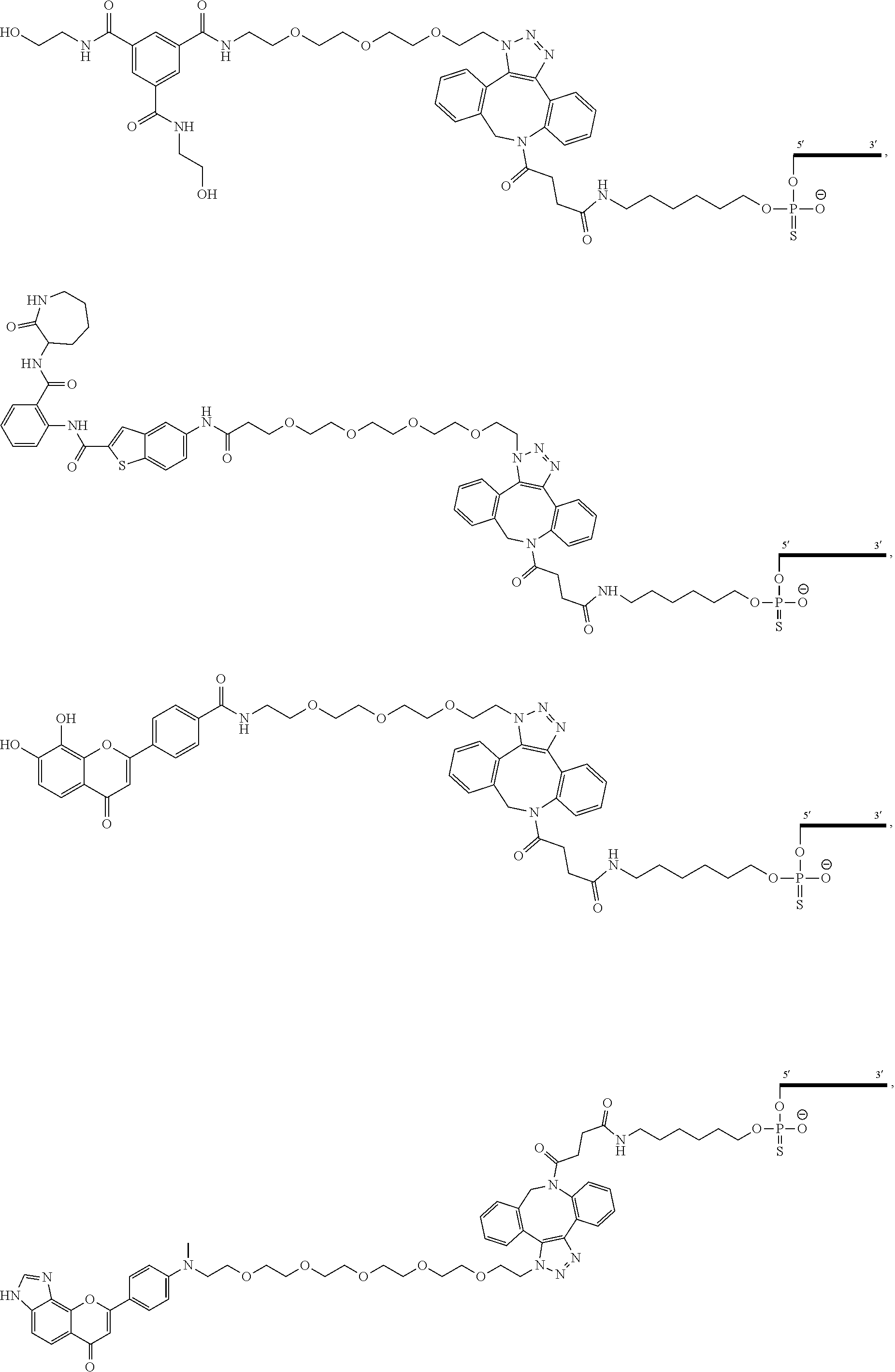 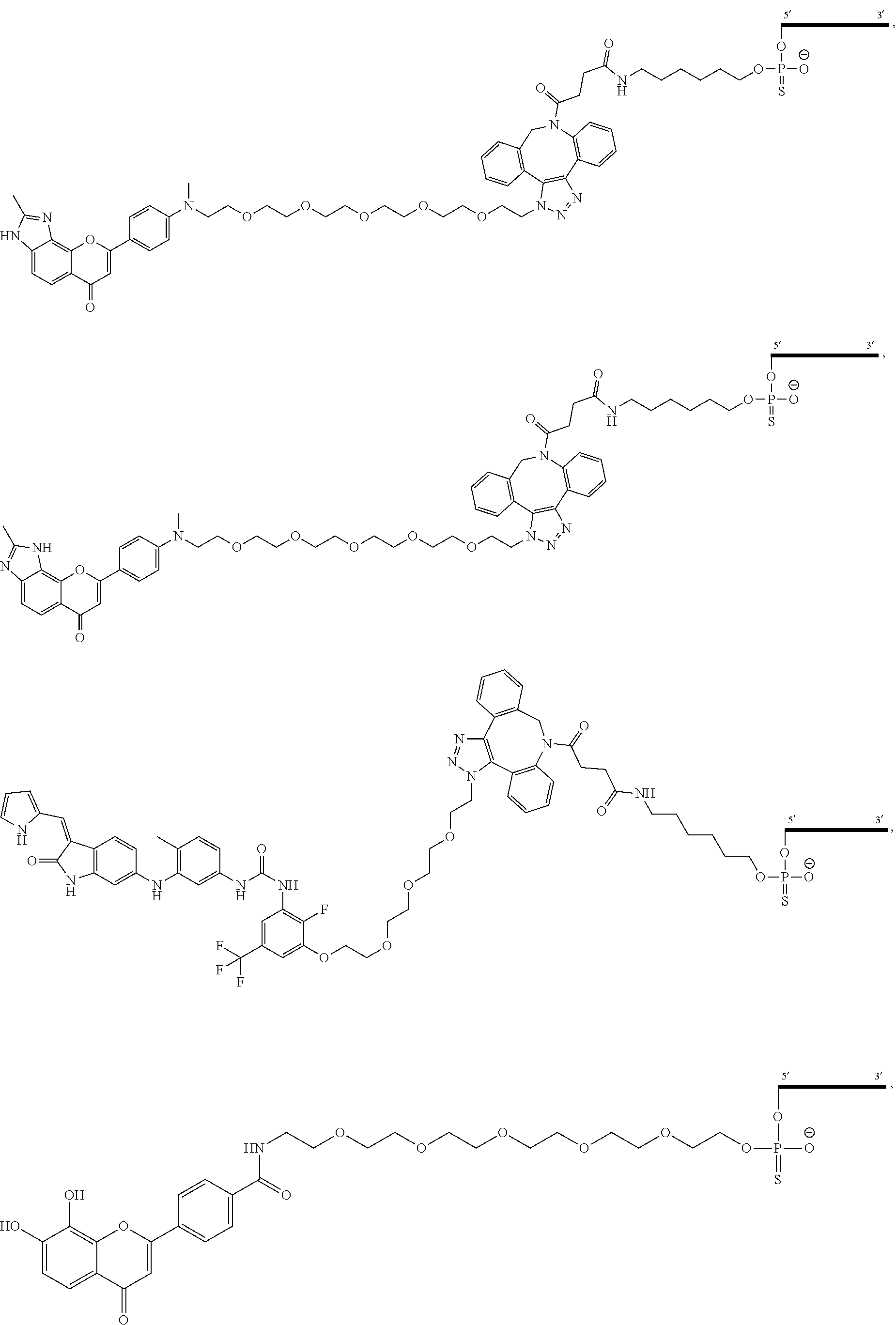 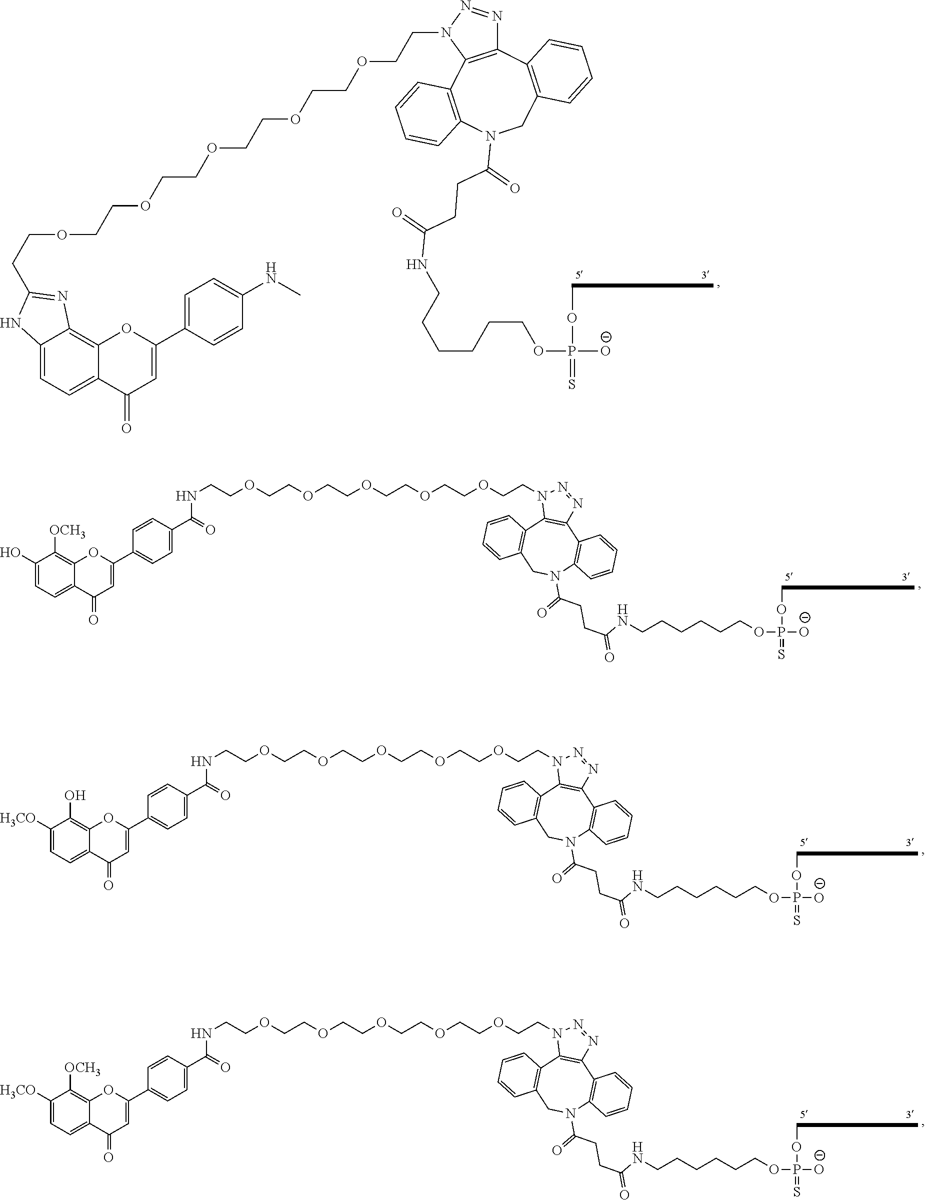 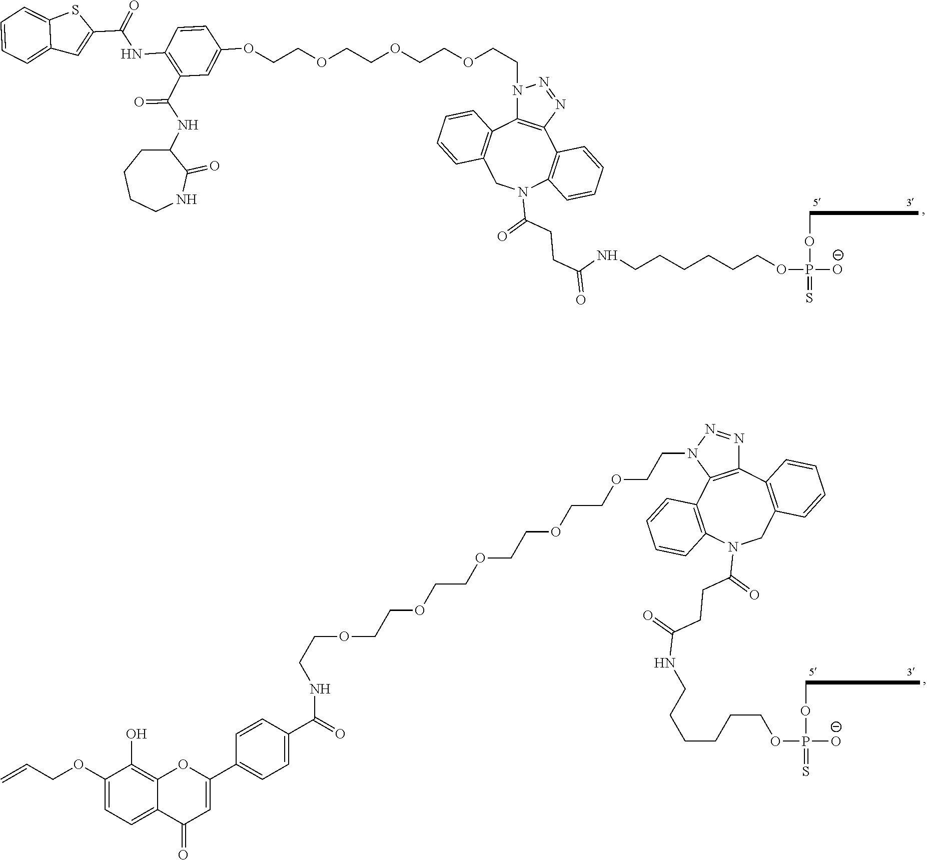 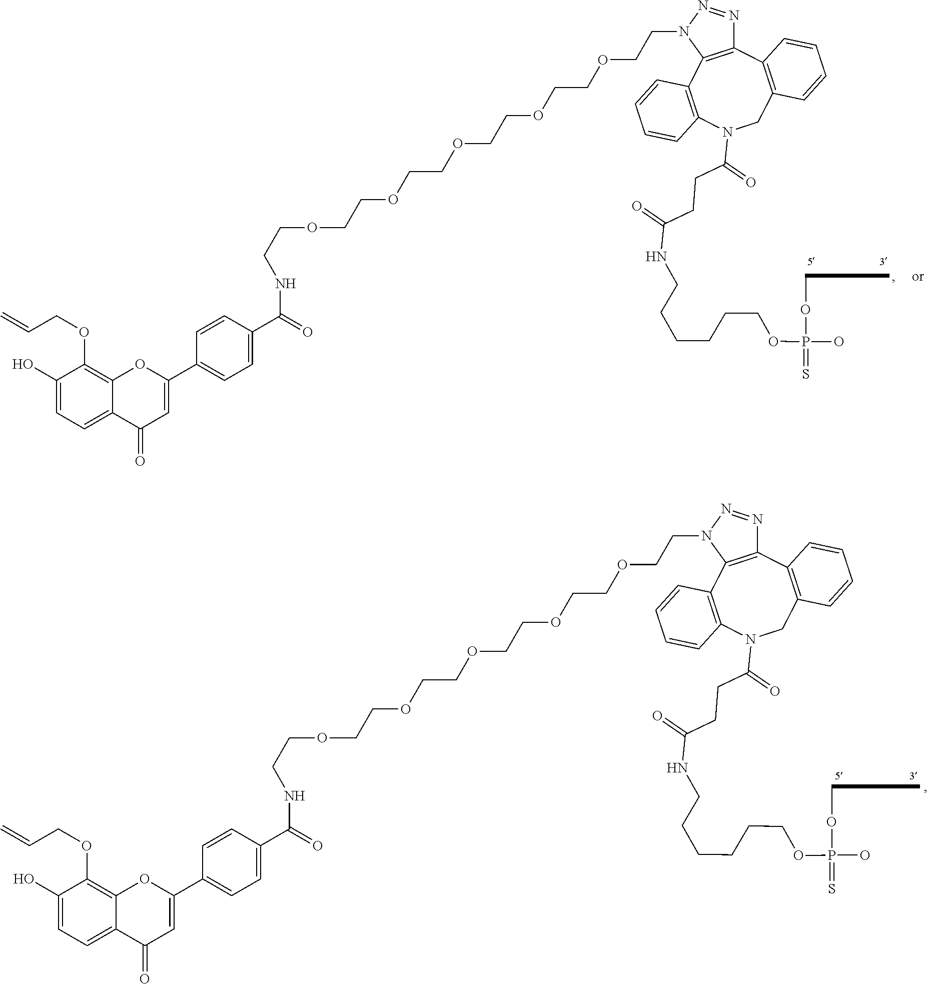
wherein [symbol] is an oligonucleotide.

114. A compound, or a stereoisomer, tautomer, prodrug, or salt thereof, wherein the compound comprises the structure: 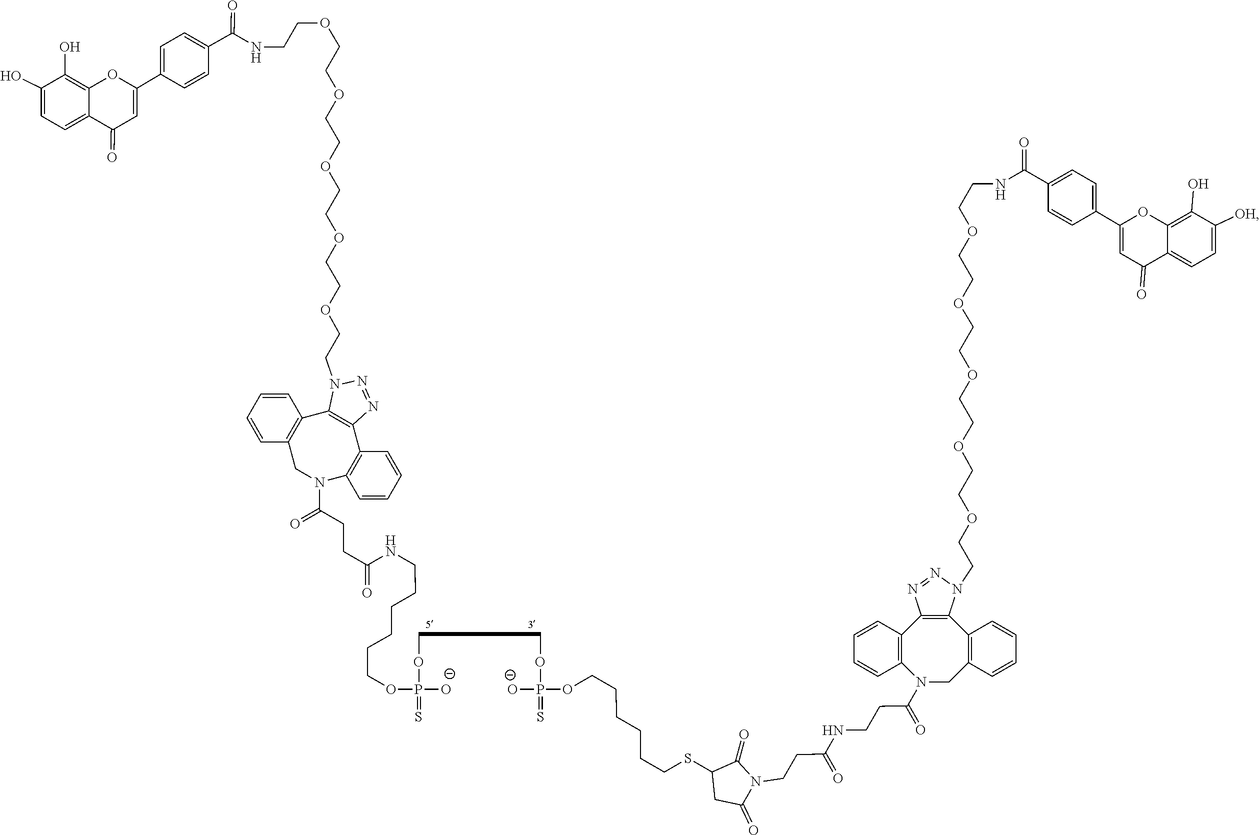 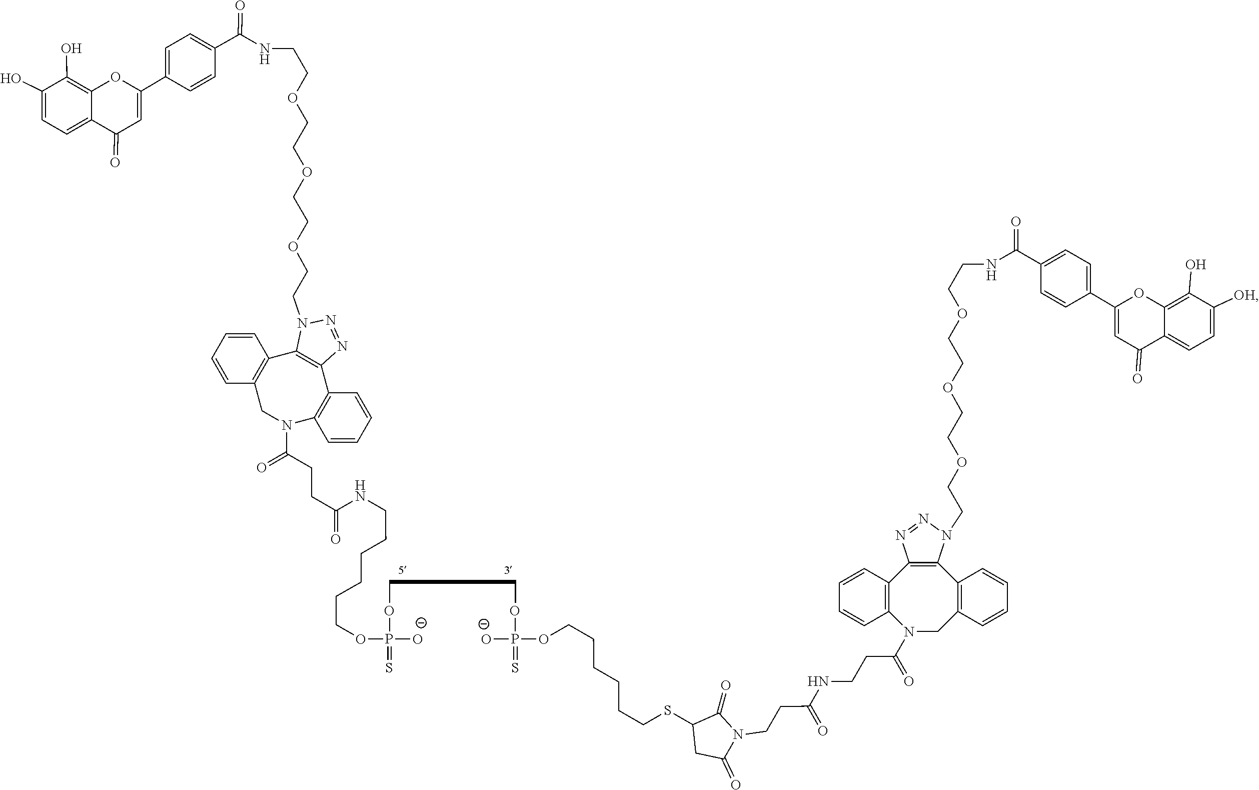 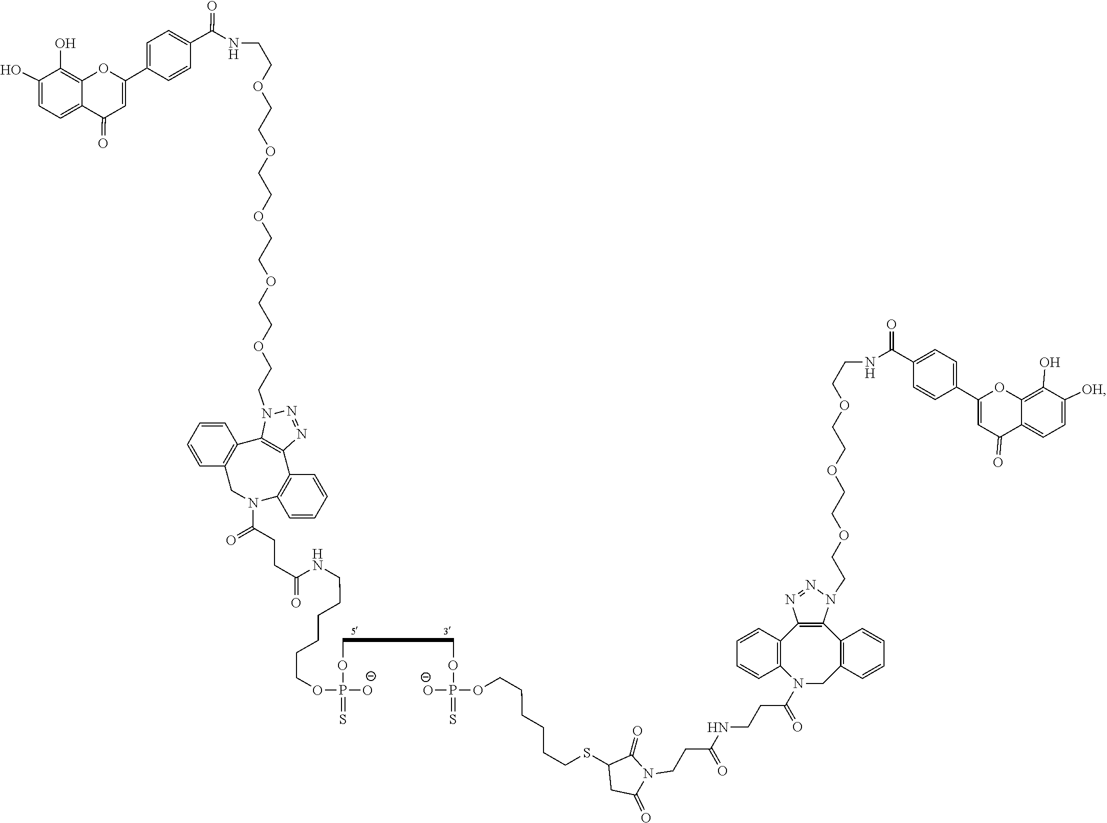 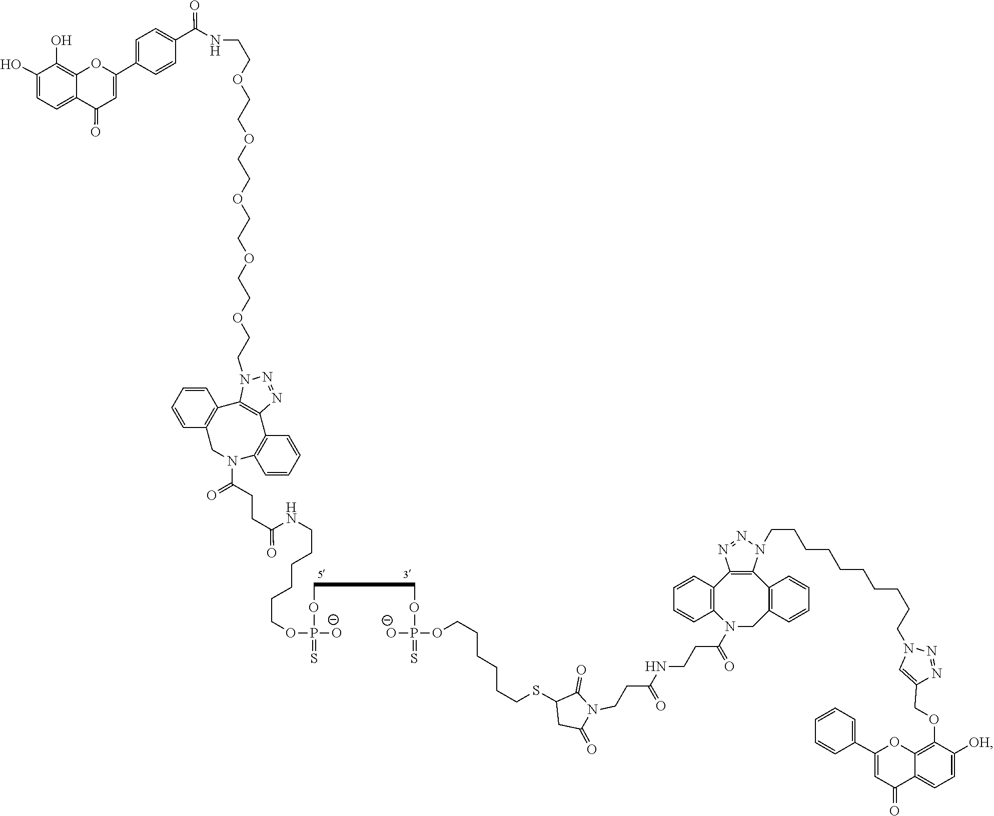 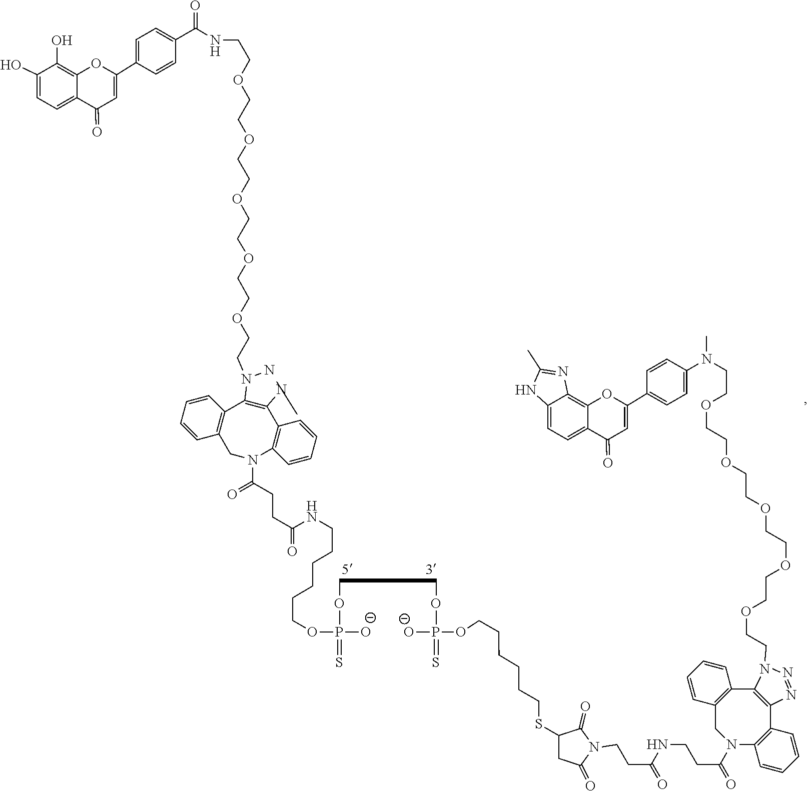 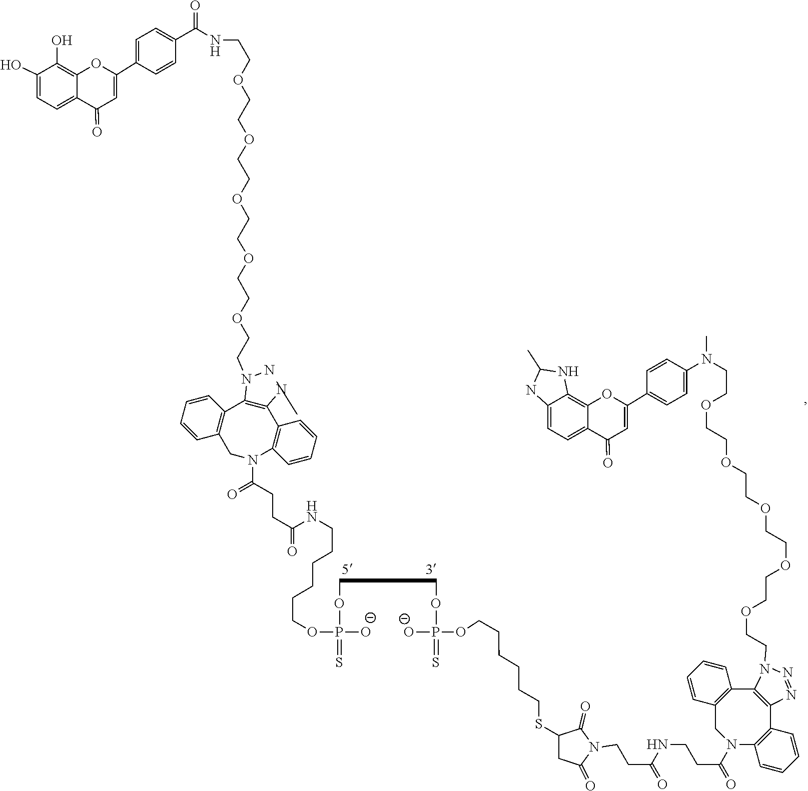 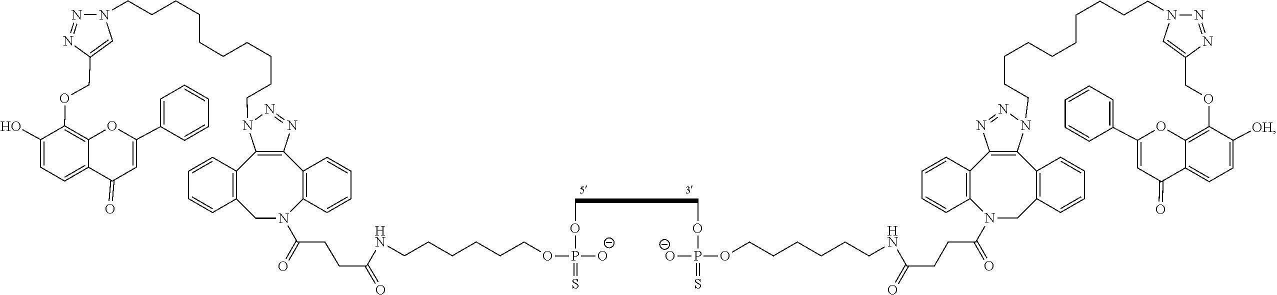 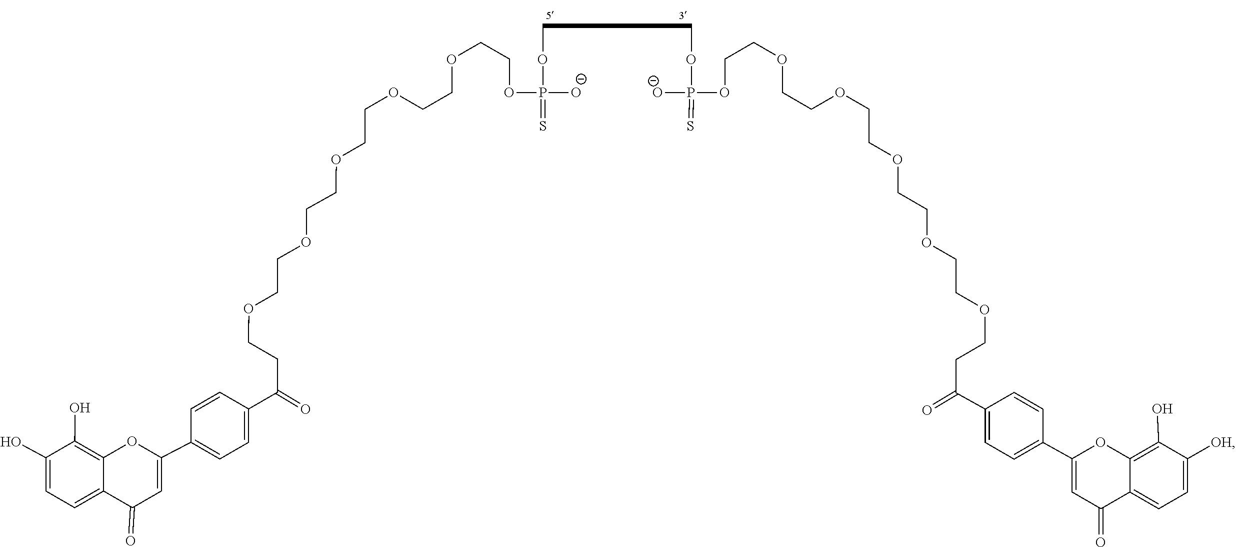
wherein [symbol] is an oligonucleotide.

115. The compound, or a stereoisomer, tautomer, prodrug, or salt thereof, of any one of claims 1-114, wherein the compound is a salt.

116. The compound, or a stereoisomer, tautomer, prodrug thereof, of claim 115, wherein the salt is a potassium salt or a sodium salt.

117. A composition comprising a compound, or a stereoisomer, tautomer, prodrug, or salt thereof, of any one of claims 1-116, and a pharmaceutically acceptable excipient.

118. A method for delivering a therapeutic oligonucleotide to the brain of a subject, comprising administration of a compound, or a stereoisomer, tautomer, prodrug, or salt thereof, of any one of claims 1-116, or a composition of claim 117, to the subject.

119. The method of claim 118, wherein the therapeutic oligonucleotide is delivered to one or more brain regions selected from the group consisting of the striatum, the cerebellum, the brain stem, the hippocampus, the frontal cortex, and the spinal cord.

120. A method for treating or ameliorating a disease, disorder, or symptom thereof in a subject, comprising administration of a compound, or a stereoisomer, tautomer, prodrug, or salt thereof, of any one of claims 1-116, or a composition of claim 117, to the subject.

121. The method of claim 120, wherein the disease, disorder, or symptom thereof is a central nervous system (CNS) disease, disorder, or symptom thereof.

122. The method of claim 120 or 121, wherein the disease, disorder, or symptom thereof is Alzheimer's disease, or a symptom thereof.

123. The method of any one of claims 118-122, wherein the administration is intrathecal administration or intracerebroventricular (ICV) administration.

124. A method of delivering one or more cargo molecules to a cell or tissue of a subject in vivo, comprising administering to the subject a compound of any one of claims 1-116 or a composition of claim 117.

125. The method of claim 124, wherein the cell or tissue is CNS cell or tissue.

126. A precursor compound, or a stereoisomer, tautomer, or salt thereof, of any one of structural Formulae (A)-(F): 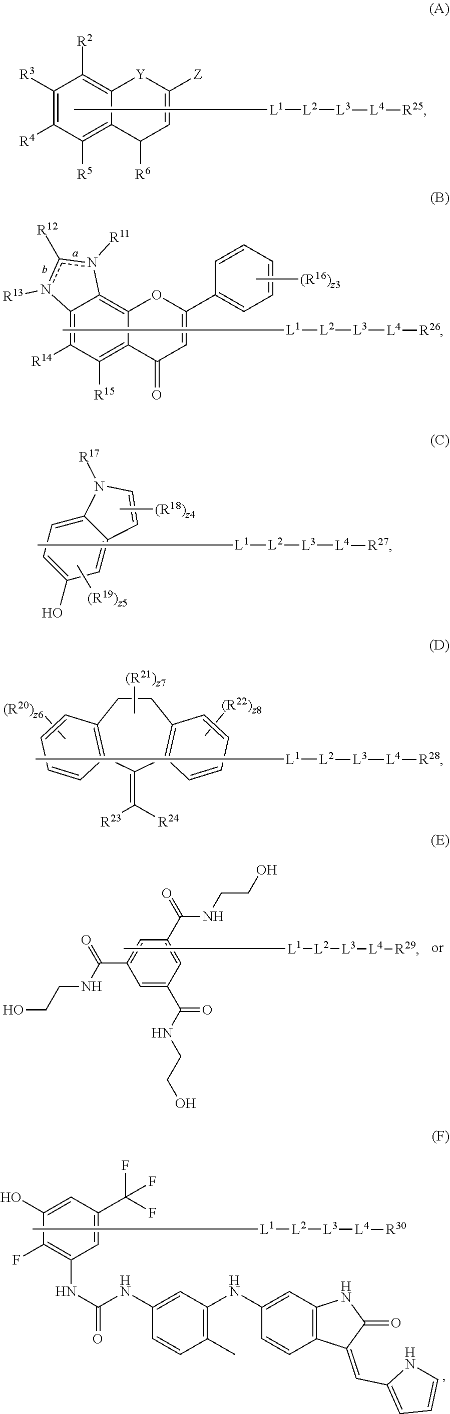
wherein:
each of L¹, L², L³, and L⁴ is independently a linker, bond, or absent;
R² is hydrogen, —OR⁷, —SR⁸, or —NR⁹R¹⁰;
R³ is hydrogen, —OR³¹, —SR³², or —NR³³R³⁴,
R⁴ is hydrogen, —OR³⁵, —SR³⁶, or —NR³⁷R³⁸;
R⁵ is hydrogen, —OR³⁹, —SR⁴⁰, or —NR⁴¹R⁴²;
R⁶ is hydrogen, —OH, optionally substituted —O-alkyl, optionally substituted —OAc, —NH₂, optionally substituted —NHAc, —SH, or ═O;
R⁷, R⁸, R⁹, R¹⁰, R³¹, R³², R³³, R³⁴, R³⁵, R³⁶, R³⁷, R³⁸, R³⁹, R⁴⁰, R⁴¹, and R⁴² are each independently hydrogen, optionally substituted alkyl, optionally substituted heteroalkyl, optionally substituted cycloalkyl, optionally substituted heterocycloalkyl, optionally substituted aryl, optionally substituted heteroaryl;
Y is CH₂, NH, S, or O;
Z is optionally substituted aryl or optionally substituted heteroaryl;
R¹¹ and R¹³ are each independently absent, hydrogen, or optionally substituted alkyl;
R¹², R¹⁴, and R¹⁵ are each independently hydrogen, optionally substituted alkyl, optionally substituted heteroalkyl, optionally substituted cycloalkyl, optionally substituted heterocycloalkyl, optionally substituted aryl, or optionally substituted heteroaryl;
R¹⁶ is hydrogen, halogen, CN, —N₃, —SOₙ₁₆R^{1A}, —SOᵥ₁₆NR^{16B}R^{16C}, —NHNR^{16B}R^{16C}, —ONR^{16B}R^{16C}, —NHC(O)NHNR^{16B}R^{16C}, —NHC(O)NR^{16B}R^{16C}, —N(O)ₘ₁₆, —NR^{16B}R^{16C}, —C(O)R^{16D}, —C(O)OR^{16D}, —C(O)NR^{16B}R^{16C}, —OR^{16A}, —NR^{16B}SO₂R^{16A}, —NR^{16B}C(O)R^{16D};
—NR^{16B}C(O)OR^{16D}, —NR^{16B}OR^{16D}, optionally substituted alkyl, optionally substituted heteroalkyl, optionally substituted cycloalkyl, optionally substituted heterocycloalkyl, optionally substituted aryl, or optionally substituted heteroaryl; [symbol]
and [symbol] are each independently a single bond or a double bond, wherein if [symbol] is a single a bond, then [symbol] is a double bond and R¹³ is absent; and further wherein if [symbol] is a single bond, then [symbol] is a double bond and R¹¹ is absent;
R^{16A}, R^{16B}, R^{16C}, R^{16D} are each independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{16B} and R^{16C} substituents bonded to the same nitrogen atom may optionally be joined to form a substituted or unsubstituted heterocycloalkyl or substituted or unsubstituted heteroaryl;
R¹⁷, R¹⁸, and R¹⁹ are each independently hydrogen, optionally substituted alkyl, optionally substituted heteroalkyl, optionally substituted cycloalkyl, optionally substituted heterocycloalkyl, optionally substituted aryl, or optionally substituted heteroaryl;
R²⁰ is hydrogen, halogen, —CN, —N₃, —SOₙ₂₀R^{1A}, —SOᵥ₂₀NR^{20B}R^{20C}, —NHNR^{20B}R^{20C}, —ONR^{20B}R^{20C}, —NHC(O)NHNR^{20B}R^{20C}, —NHC(O)NR^{20B}R^{20C}, —N(O)ₘ₂₀, —NR^{20B}R^{20C}, —C(O)R^{20D}, —C(O)OR^{20D}, —C(O)NR^{20B}R^{20C}, —OR^{20A}, —NR^{20B}SO₂R^{20A}, —NR^{20B}C(O)R^{20D};
—NR^{20B}C(O)OR^{20D}, —NR^{20B}OR^{20D}, optionally substituted alkyl, optionally substituted heteroalkyl, optionally substituted cycloalkyl, optionally substituted heterocycloalkyl, optionally substituted aryl, or optionally substituted heteroaryl;
R²¹ is hydrogen, halogen, —CN, —N₃, —SOₙ₂₁R^{1A}, —SOᵥ₂₁NR^{21B}R^{21C}, —NHNR^{21B}R^{21C}, —ONR^{21B}R^{21C}, —NHC(O)NHNR^{21B}R^{21C}, —NHC(O)NR^{21B}R^{21C}, —N(O)ₘ₂₁, —NR^{21B}R^{21C}, —C(O)R^{21D}, —C(O)OR^{21D}, C(O)NR^{21B}R^{21C}, —OR^{21A}, —NR^{21B}SO₂R^{21A}, —NR^{21B}C(O)R^{21D};
—NR^{21B}C(O)OR^{21D}, —NR^{21B}OR^{21D}, optionally substituted alkyl, optionally substituted heteroalkyl, optionally substituted cycloalkyl, optionally substituted heterocycloalkyl, optionally substituted aryl, or optionally substituted heteroaryl;
R²² and R²³ are each independently hydrogen, optionally substituted alkyl, optionally substituted heteroalkyl, optionally substituted cycloalkyl, optionally substituted heterocycloalkyl, optionally substituted aryl, or optionally substituted heteroaryl;
R²⁴ is hydrogen, halogen, —CN, —N₃, —SOₙ₂₄R^{1A}, —SOᵥ₂₄NR^{24B}R^{24C}, —NHNR^{24B}R^{24C}, —ONR^{24B}R^{24C}, —NHC(O)NHNR^{24B}R^{24C}, —NHC(O)NR^{24B}R^{24C}, —N(O)ₘ₂₄, —NR^{24B}R^{24C}, —C(O)R^{24D}, —C(O)OR^{24D}, —C(O)NR^{24B}R^{24C}, OR^{24A}, —NR^{24B}SO₂R^{24A}, —NR^{24B}C(O)R^{24D};
—NR^{24B}C(O)OR^{24D}, —NR^{24B}OR^{24D}, optionally substituted alkyl, optionally substituted heteroalkyl, optionally substituted cycloalkyl, optionally substituted heterocycloalkyl, optionally substituted aryl, or optionally substituted heteroaryl;
R^{20A}, R^{20B}, R^{20C}, R^{20D}, R^{21A}, R^{21B}, R^{21C}, R^{21D}, R^{24A}, R^{24B}, R^{24C}, and R^{24D} are each independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{20B}, R^{20C}, R^{21B}, R^{21C}, R^{24B}, R^{24C}, R^{24B}, and R^{24C} substituents bonded to the same nitrogen atom may optionally be joined to form a substituted or unsubstituted heterocycloalkyl or substituted or unsubstituted heteroaryl;
n16, n20, n21, n23, n24, z6, and z8 are each independently 0, 1, 2, 3, or 4;
v16, v20, v21, m16, m20, m21, and m24 are each independently 1 or 2; and
z3 is 0, 1, 2, 3, 4, or 5;
z4 and z7 are each independently 0, 1, or 2;
z5 is 0, 1, 2, or 3;
z6 and z8 are each independently 0, 1, 2, 3, or 4 and
R²⁵, R²⁶, R²⁷, R²⁸, R²⁹, R³⁰ are each independently —N₃.

127. A method for making a compound, or a stereoisomer, tautomer, prodrug, or salt thereof, of any one of claims 1-116, comprising contacting the precursor compound of claim 126 with a compound of structural Formula (G) and/or (H): 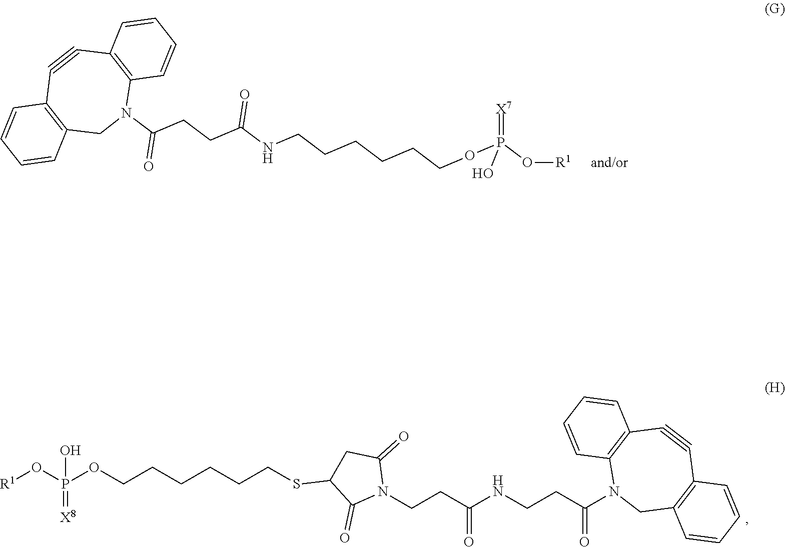
or
a salt thereof,
wherein:
X⁷ and X⁸ are each independently O or S; and
R¹ is an oligonucleotide, a protecting group, a small molecule, a protein, an antibody, or a peptide.
